(19) United States
(12) Patent Application Publication
(10) Pub. No.: US 2021/0299070 A1
(43) Pub. Date: Sep. 30, 2021

(54) METHODS OF TREATING CREATINE TRANSPORTER DEFICIENCY

(71) Applicant: Jnana Therapeutics, Inc., Boston, MA (US)

(72) Inventors: Giovanni Muncipinto, Cambridge, MA (US); Lyn H. Jones, Winchester, MA (US); Terry J. Rettenmaier, Braintree, MA (US)

(21) Appl. No.: 17/278,051

(22) PCT Filed: Sep. 20, 2019
(86) PCT No.: PCT/US19/52198

§ 371 Date: Mar. 19, 2021

Related U.S. Application Data
(60) Provisional application No. 62/734,601, filed on Sep. 21, 2018.

(51) Int. Cl. A61K 31/166; A61K 31/277; A61K 31/4402; A61K 31/455; A61K 31/513; A61K 31/337; A61K 31/341; A61K 31/4409; A61K 31/44; A61K 31/4168; A61K 31/505; A61K 31/444; A61K 31/407; A61K 31/396; A61K 31/421; A61K 31/36; A61K 31/4439; A61K 31/4184; A61K 31/506; A61K 31/4178; A61K 31/4164; A61K 31/4709; A61K 31/47; A61K 31/397; A61K 31/497

(52) U.S. Cl. CPC A61K 31/166; A61K 31/497; A61K 31/4402; A61K 31/455; A61K 31/513; A61K 31/337; A61K 31/341; A61K 31/4409; A61K 31/44; A61K 31/4168; A61K 31/505; A61K 31/444; A61K 31/407; A61K 31/396; A61K 31/421; A61K 31/36; A61K 31/4439; A61K 31/4184; A61K 31/506; A61K 31/4178; A61K 31/4164; A61K 31/4709; A61K 31/47; A61K 31/397; A61K 31/277

(57) ABSTRACT

Disclosed are methods of treating creatine transporter deficiency, comprising administering to a mammal in need thereof a therapeutically effective amount of a compound that increases transport of a substrate by a mutant or wild-type creatine transporter. Also disclosed are methods of increasing transport of guanidinoacetic acid or a salt thereof across the blood-brain barrier of a mammal, and methods of decreasing accumulation or the concentration of guanidinoacetic acid or a salt thereof in a mammalian cell.

### RELATED APPLICATIONS

This application claims the benefit of priority to U.S. Provisional Patent Application No. 62/734,601, filed Sep. 21, 2018.

### BACKGROUND

Creatine is synthesized in the liver and kidney, and transported throughout the body to tissues with high energy demands through an active transport system. Creatine is used by the body during times of increased energy demands to resynthesize ATP from ADP rapidly through the anaerobic conversion of phosphorylated creatine (phosphocreatine) to creatine via a reversible reaction catalyzed by the enzyme creatine kinase. In times of low energy demands, excess ATP can be utilized to convert creatine to phosphocreatine.

The creatine phosphate system is needed for the storage and transmission of phosphate-bound energy in the brain and muscle. The brain and muscle have particularly high metabolic demands, making creatine a necessary molecule in ATP homeostasis. In order for creatine to reach the brain, it must first pass through the blood-brain barrier (BBB). The BBB separates blood from brain interstitial fluid and, therefore, is able to regulate the transfer of nutrients to the brain from the blood. In order to pass through the BBB, creatine utilizes a creatine transporter (CRT). When present at the BBB, CRT mediates the passage of creatine from the blood to the brain. When moving from the blood to the brain, creatine is transported against the creatine concentration gradient present at the border between the brain and circulating blood.

Creatine transporter deficiency (CTD) has been reported to be the most common cerebral creatine deficiency syndrome (CCDS). Creatine transporter deficiency is an X-linked disorder caused by mutations in the SLC6A8 gene. The SLC6A8 gene, located on the short arm of the sex chromosome, provides instructions for making a protein that transports the compound creatine into cells. Creatine is needed for the body to store and use energy properly. People with CTD have intellectual disability, which can range from mild to severe, and delayed speech development. Some affected individuals develop behavioral disorders such as attention deficit hyperactivity disorder or autistic behaviors that affect communication and social interaction. They may also experience seizures. Children with CID may experience slow growth and exhibit delayed development of motor skills such as sitting and walking. CTD is difficult to treat because the actual transporter responsible for transporting creatine to the brain and muscles is defective. There is no current standard of care.

### SUMMARY

One aspect of the invention provides methods of treating creatine transporter deficiency, comprising administering to a mammal in need thereof a therapeutically effective amount of a compound or a pharmaceutical composition comprising said compound that increases cellular creatine uptake by a mutant creatine transporter.

Another aspect of the invention relates to methods of treating creatine transporter deficiency, comprising administering to a mammal in need thereof a therapeutically effective amount of a compound that increases creatine transport across the blood-brain barrier by a creatine transporter.

One aspect the invention provides methods of treating creatine transporter deficiency, comprising administering to a mammal in need thereof a therapeutically effective amount of a compound that increases transport of a substrate by a creatine transporter or protein.

Another aspect of the invention relates to methods of treating creatine transporter deficiency, comprising administering to a mammal in need thereof a therapeutically effective amount of a compound that increases transport of a substrate across the blood-brain barrier by a creatine transporter or protein.

One aspect of the invention relates to methods of treating creatine transporter deficiency, comprising administering to a mammal in need thereof a therapeutically effective amount of a compound that increases transport of a substrate across the neuronal plasma membrane by a creatine transporter or protein.

Another aspect of the invention relates to methods of decreasing accumulation or the concentration of guanidinoacetic acid or a salt thereof in a cell, comprising administering to a mammal in need thereof a therapeutically effective amount of a compound that increases transport of guanidinoacetic acid or a salt thereof by a creatine transporter or protein.

One aspect of the invention relates to methods of increasing transport of guanidinoacetic acid or a salt thereof across the blood-brain barrier, comprising administering to a mammal in need thereof a therapeutically effective amount of a compound that increases transport of guanidinoacetic acid or a salt thereof by a creatine transporter or protein.

The methods are effective for a variety of subjects including mammals, e.g., humans and other animals, such as laboratory animals, e.g., mice, rats, rabbits, or monkeys, or domesticated and farm animals, e.g., cats, dogs, goats, sheep, pigs, cows, or horses.

In certain embodiments, the subject is a mammal. In certain embodiments, the mammal is a human.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### BRIEF DESCRIPTION OF THE DRAWINGS

FIG. 1 is a table summarizing trafficking and correction assay data for exemplary compounds acting on mutant SLC6A8.

FIG. 2 is a table summarizing trafficking and correction assay data for exemplary compounds acting on mutant SLC6A8.

FIG. 3 is a table summarizing trafficking and correction assay data for exemplary compounds acting on mutant SLC6A8.

FIG. 4 is a table summarizing trafficking and correction assay data for exemplary compounds acting on mutant SLC6A8.

FIG. 5 is a table summarizing trafficking and correction assay data for exemplary compounds acting on wild-type SLC6A8.

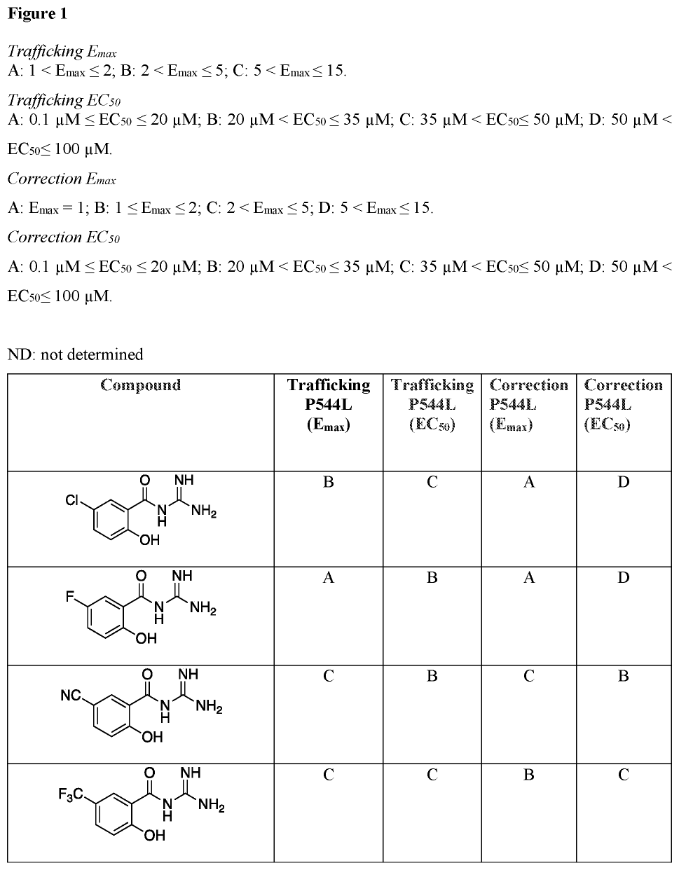

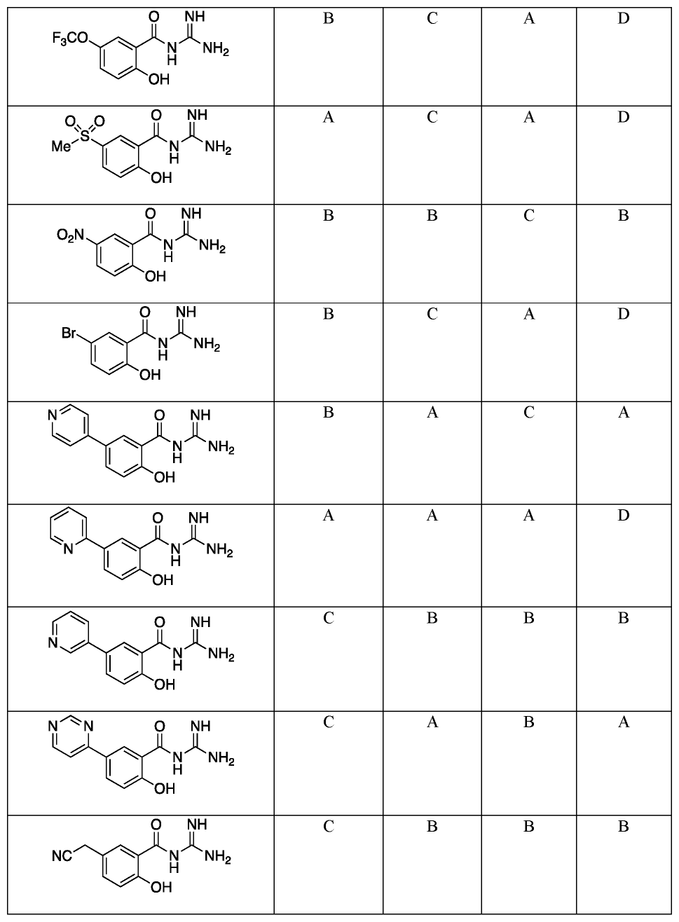

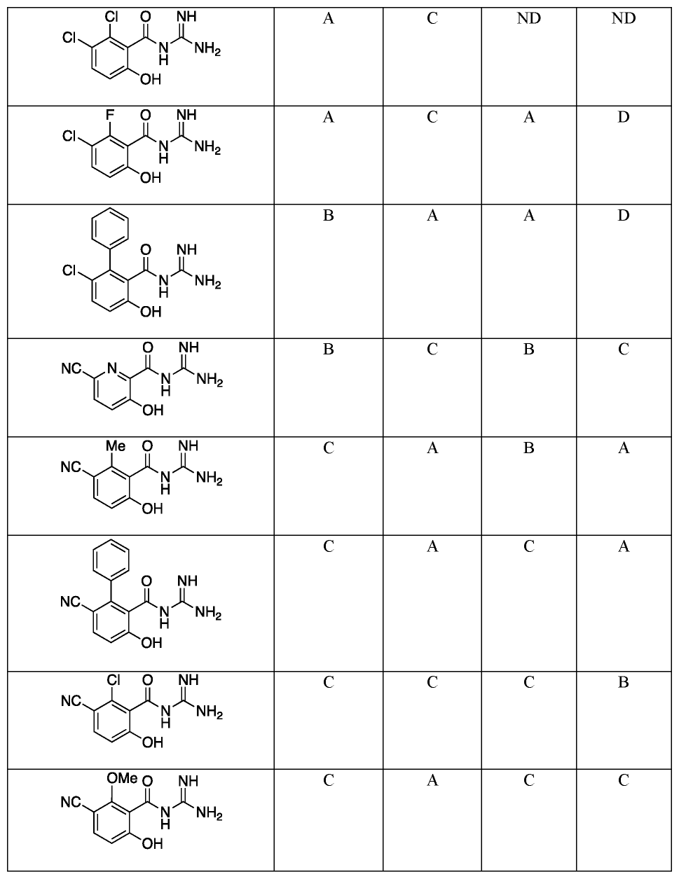

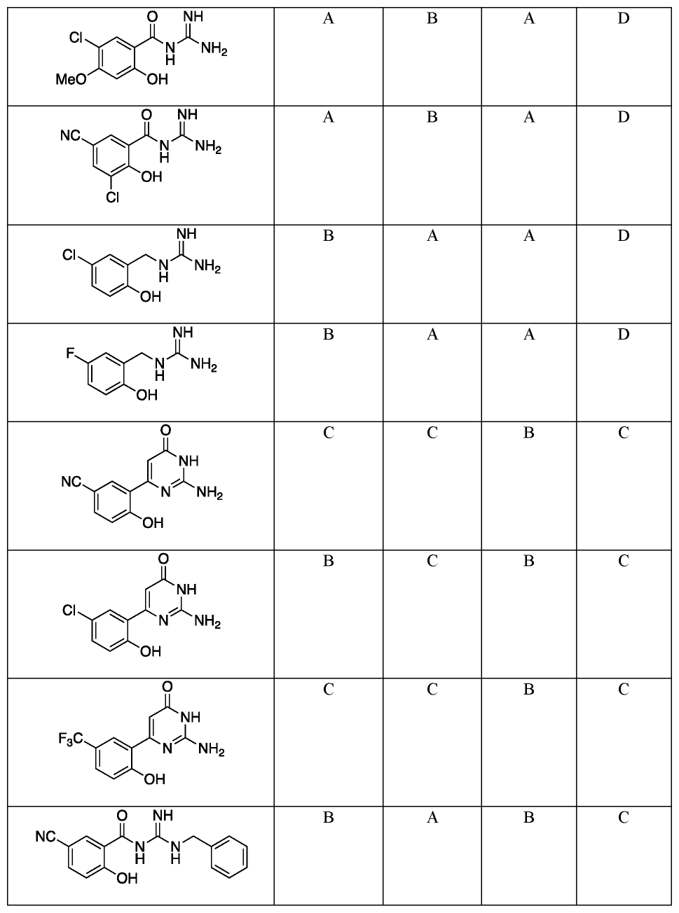

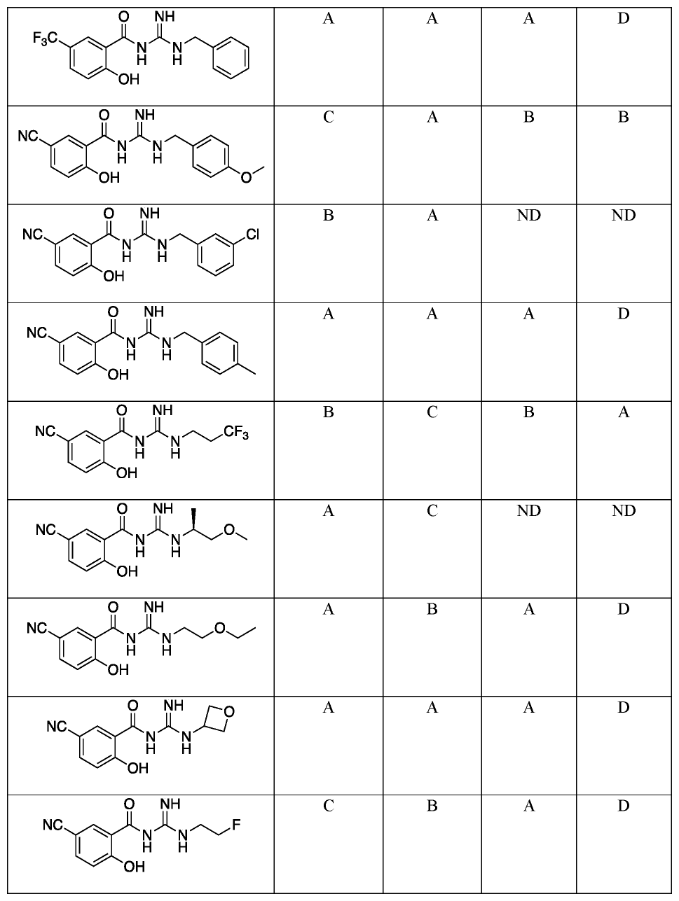

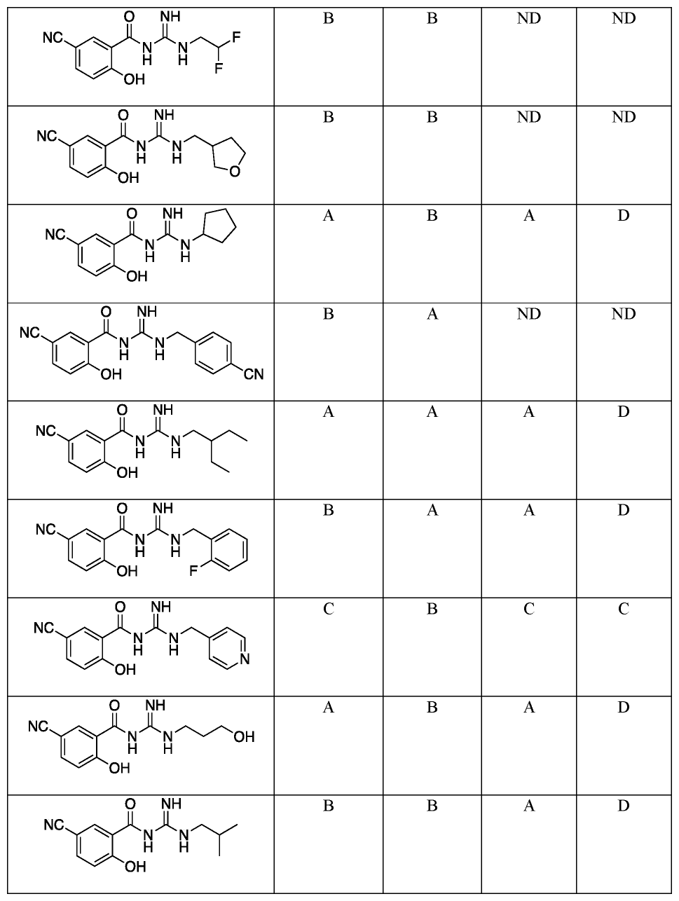

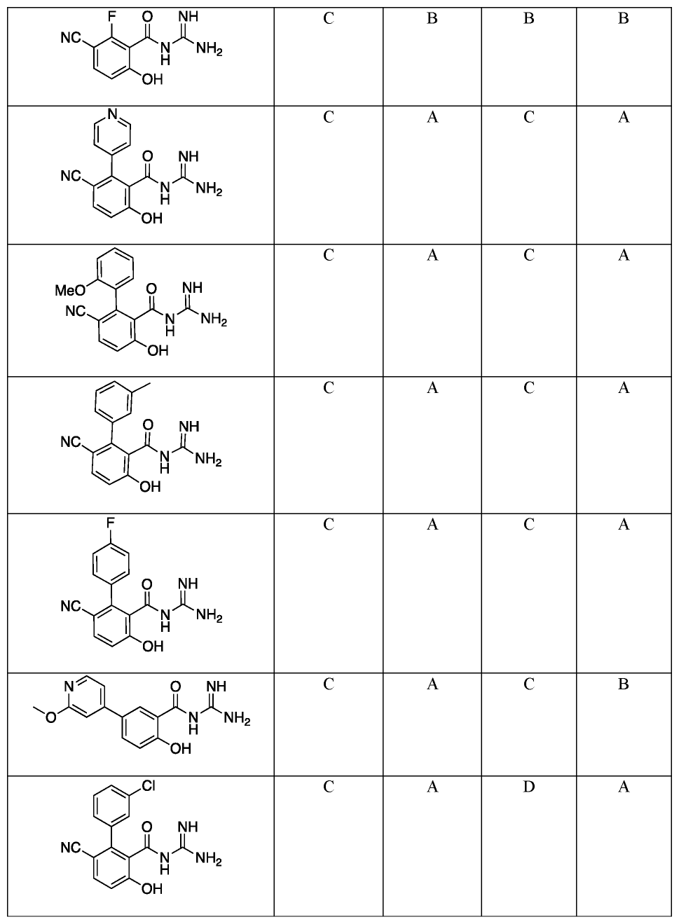

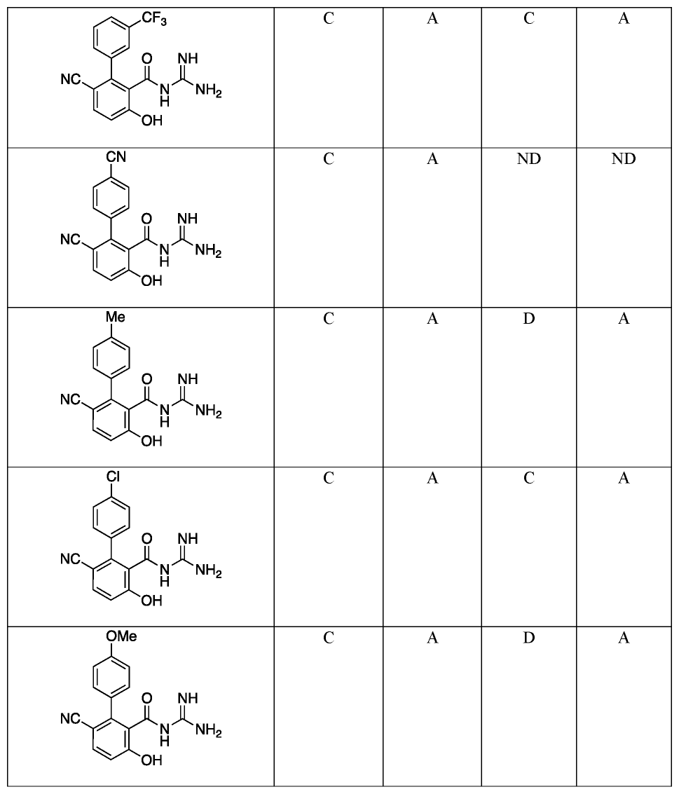

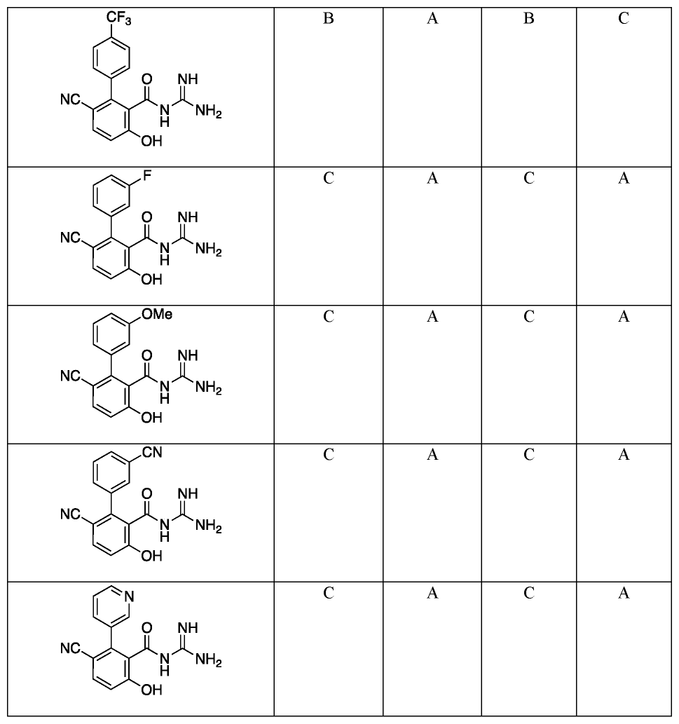

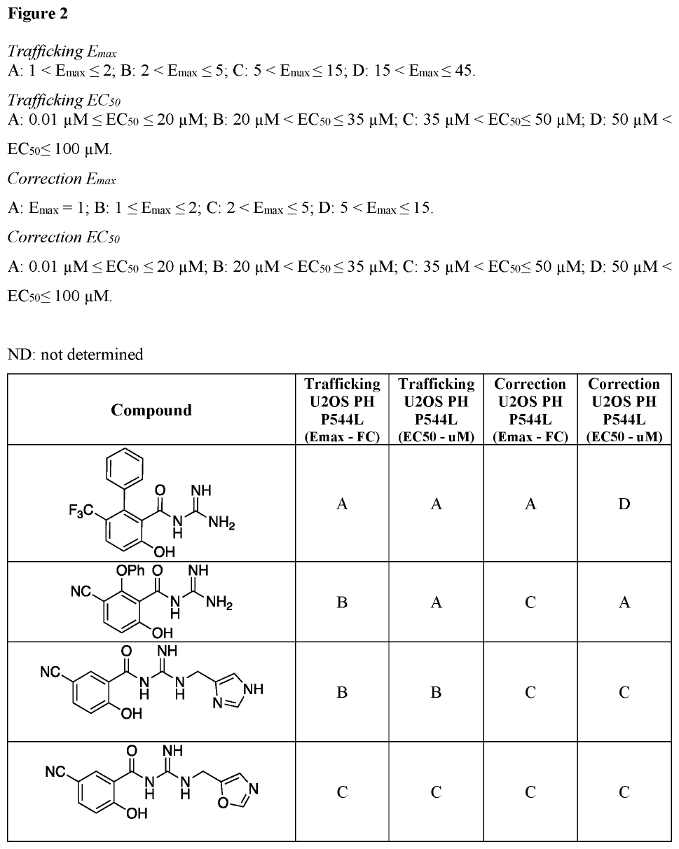

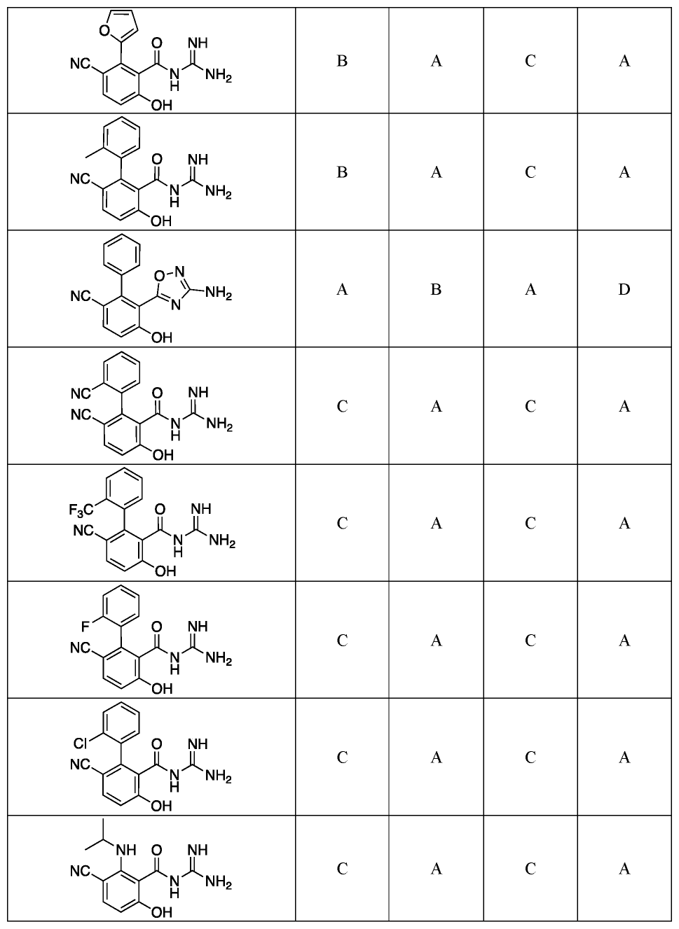

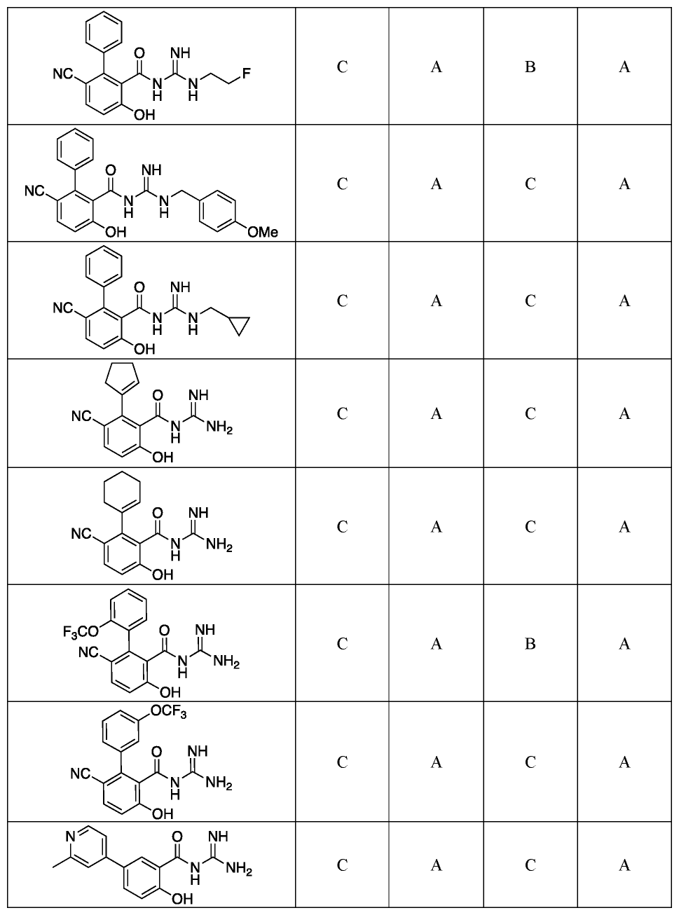

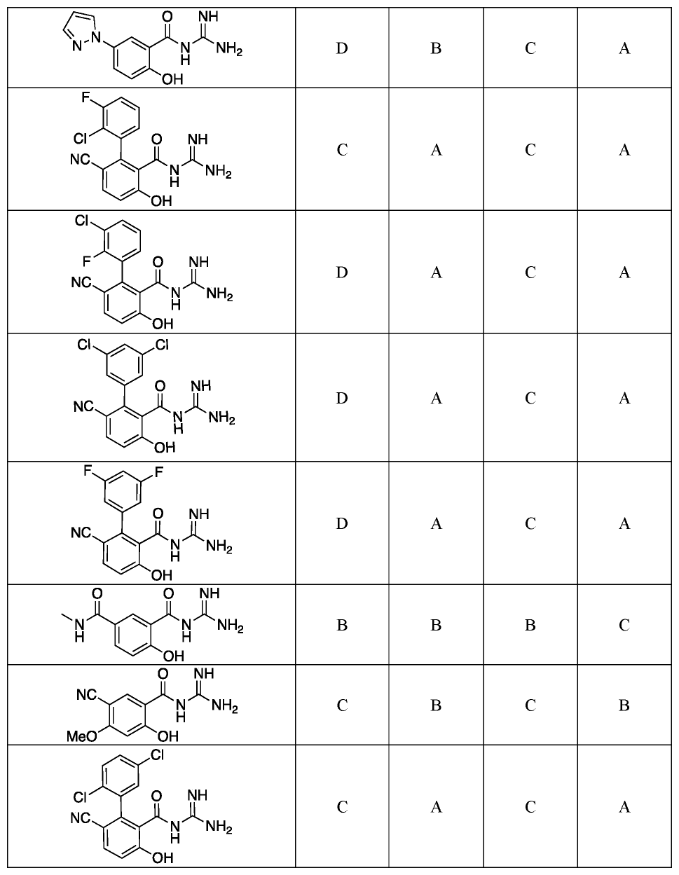

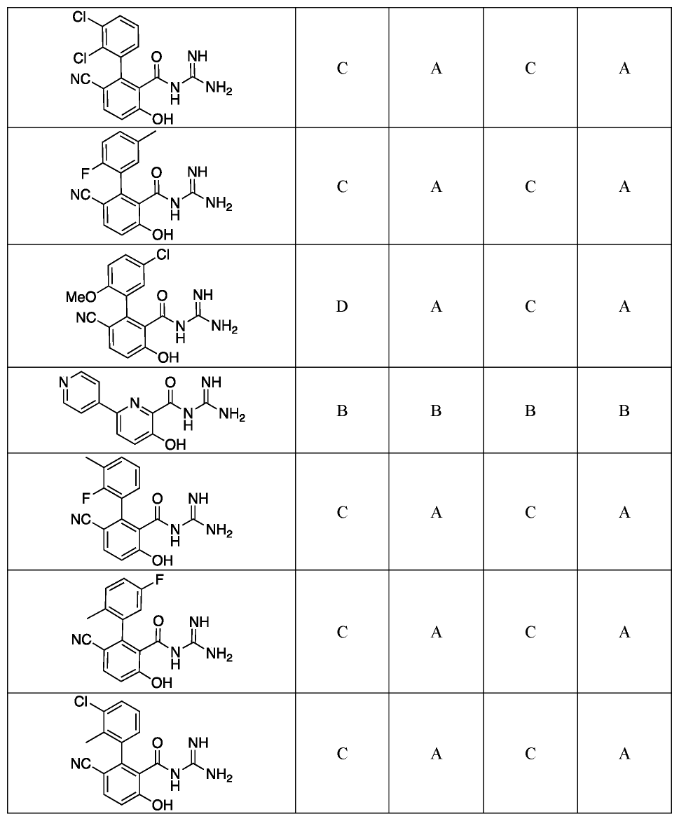

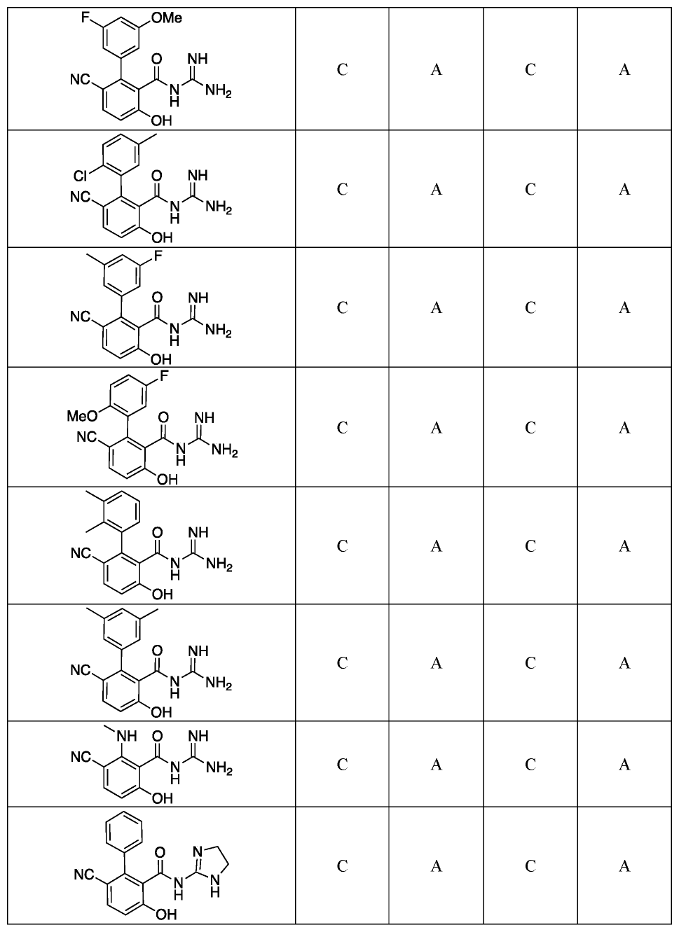

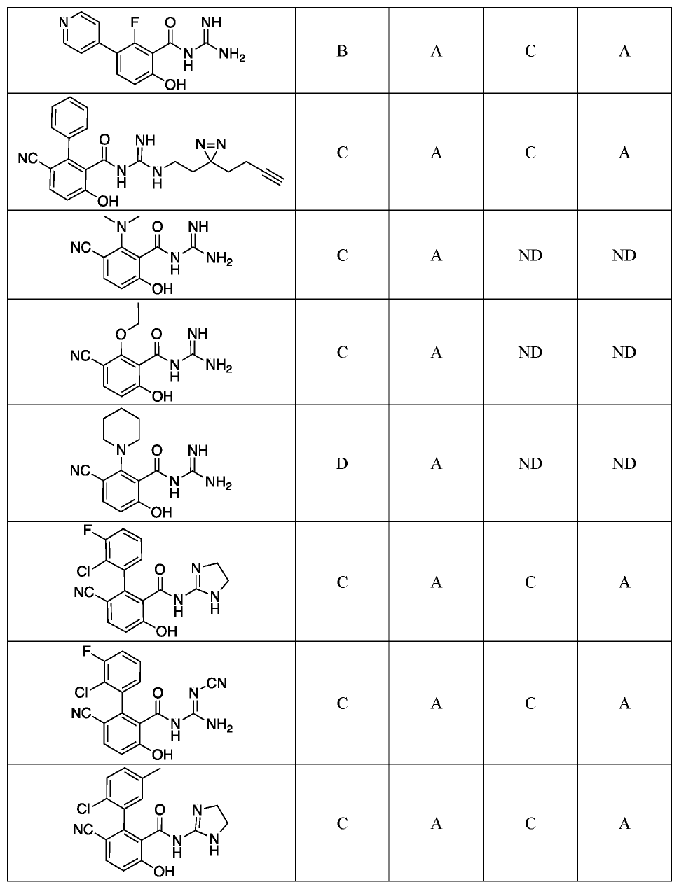

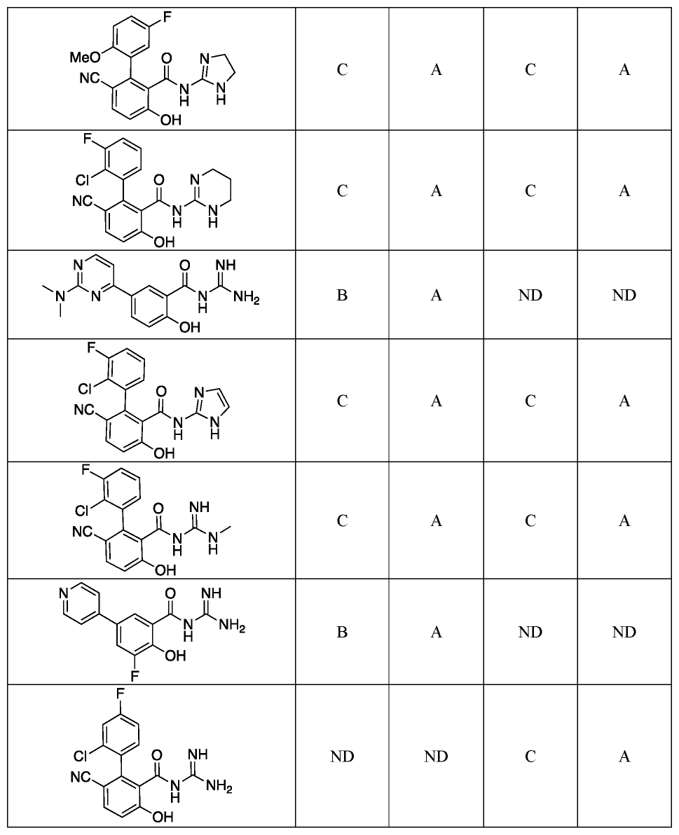

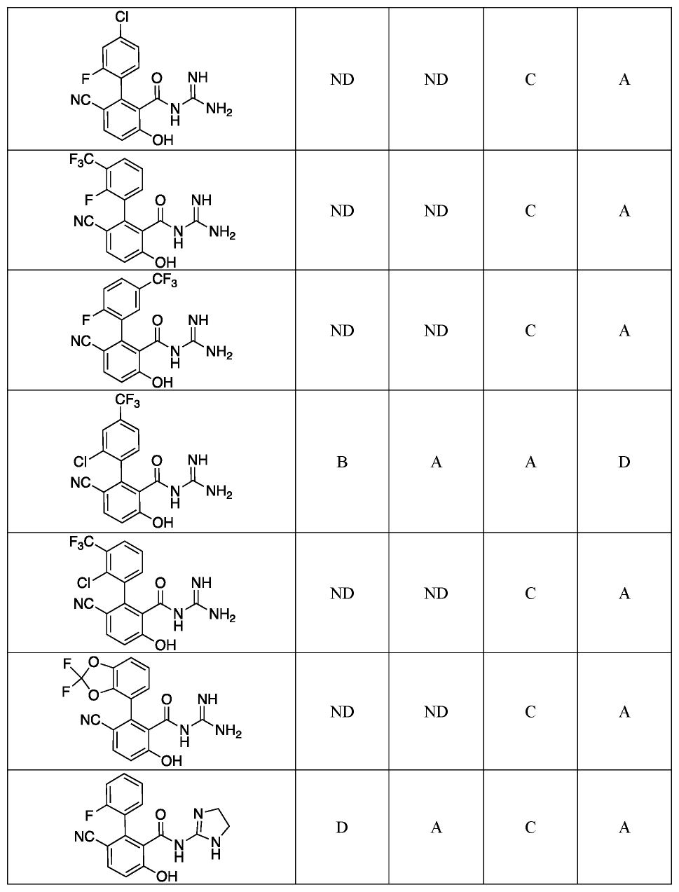

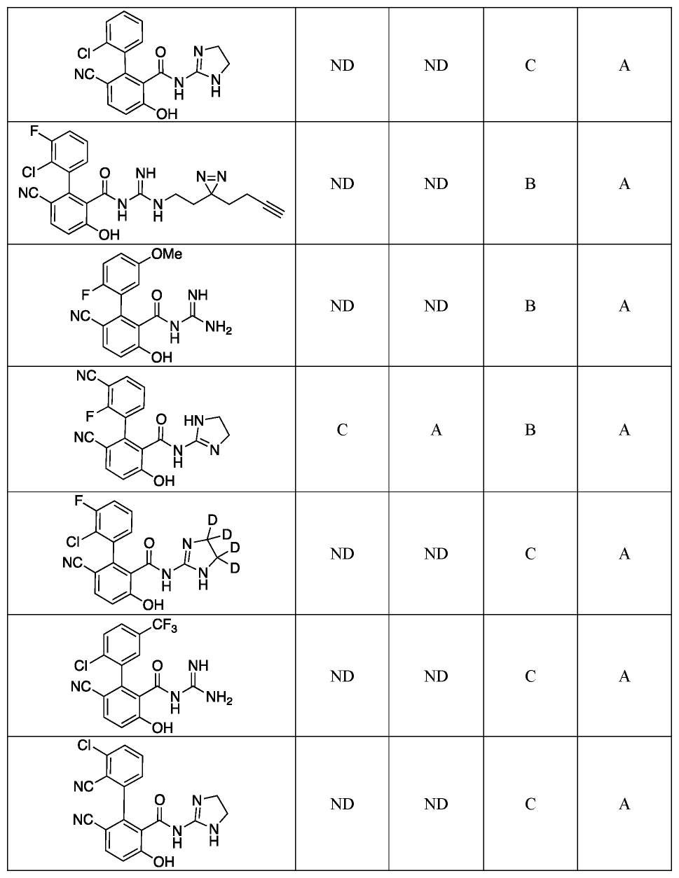

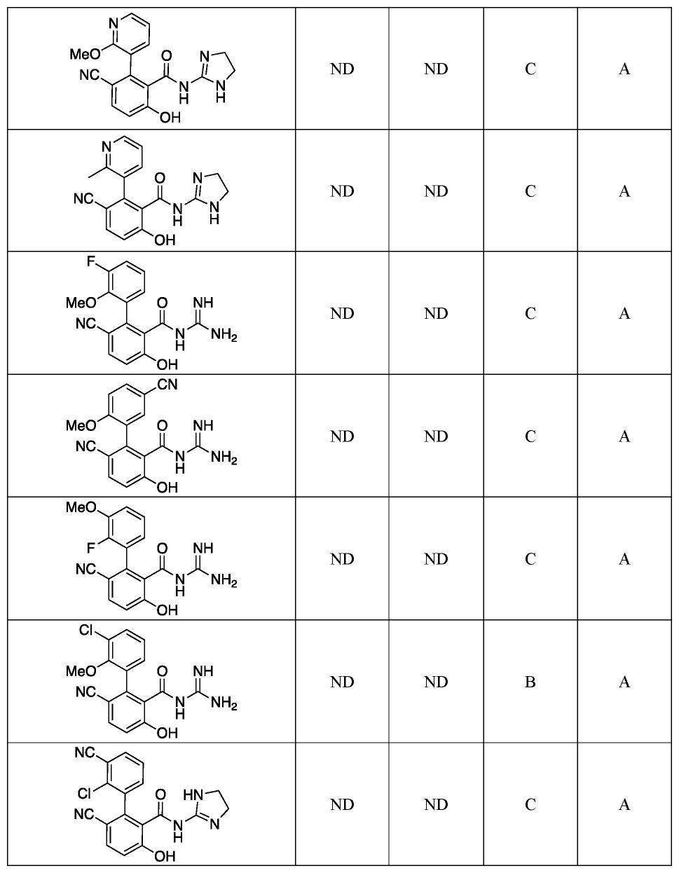

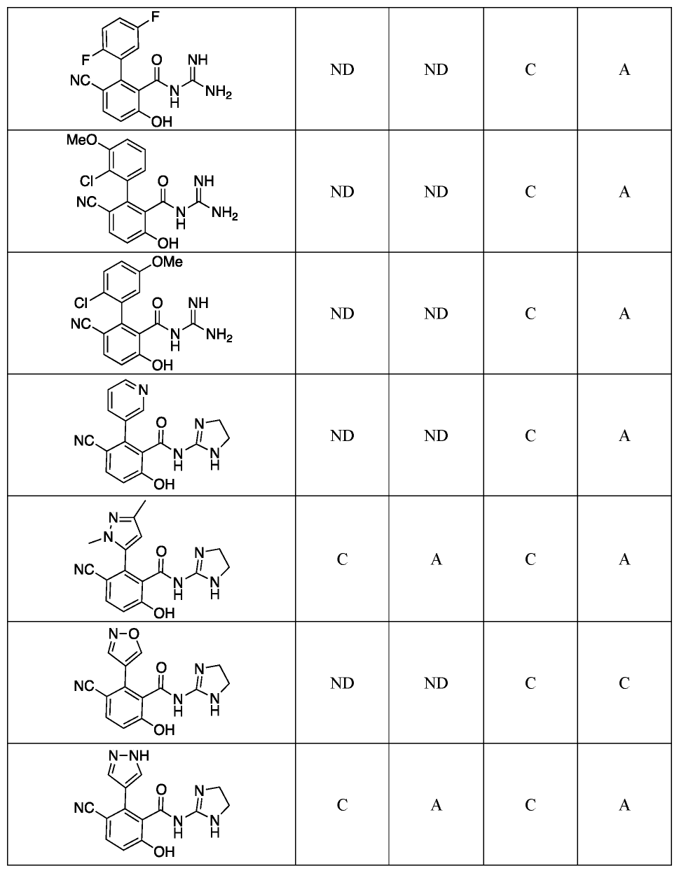

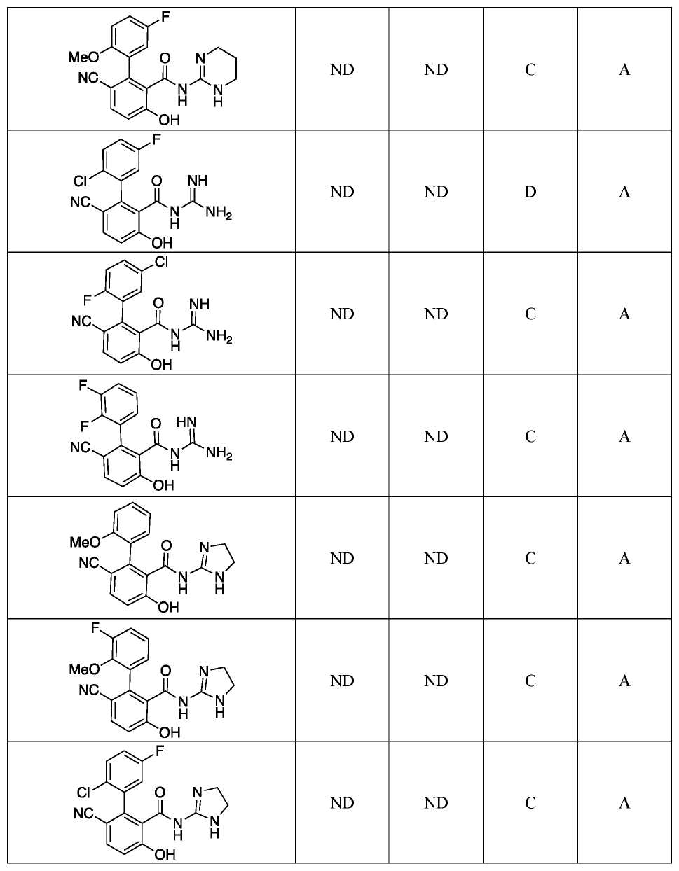

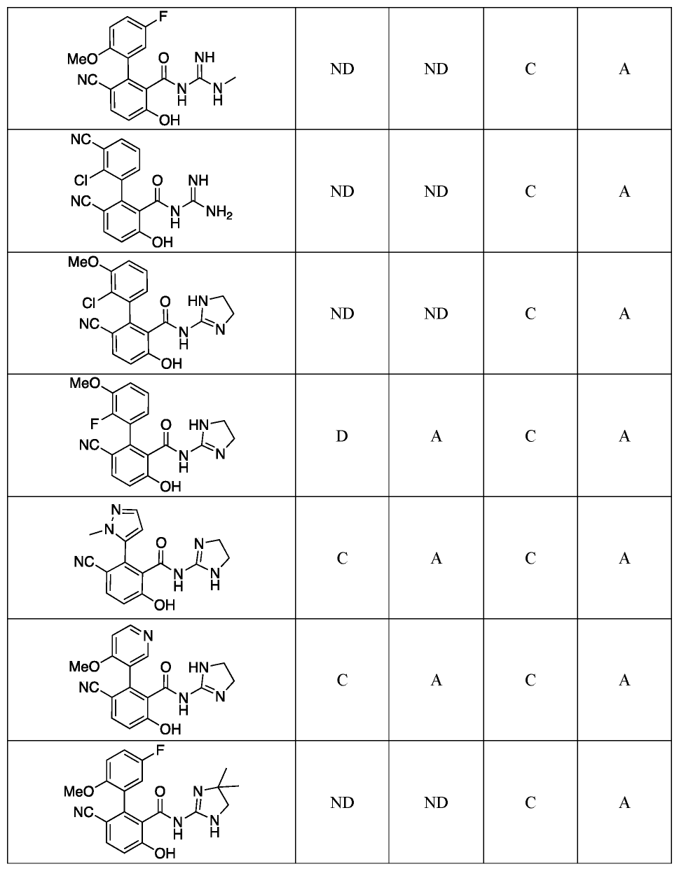

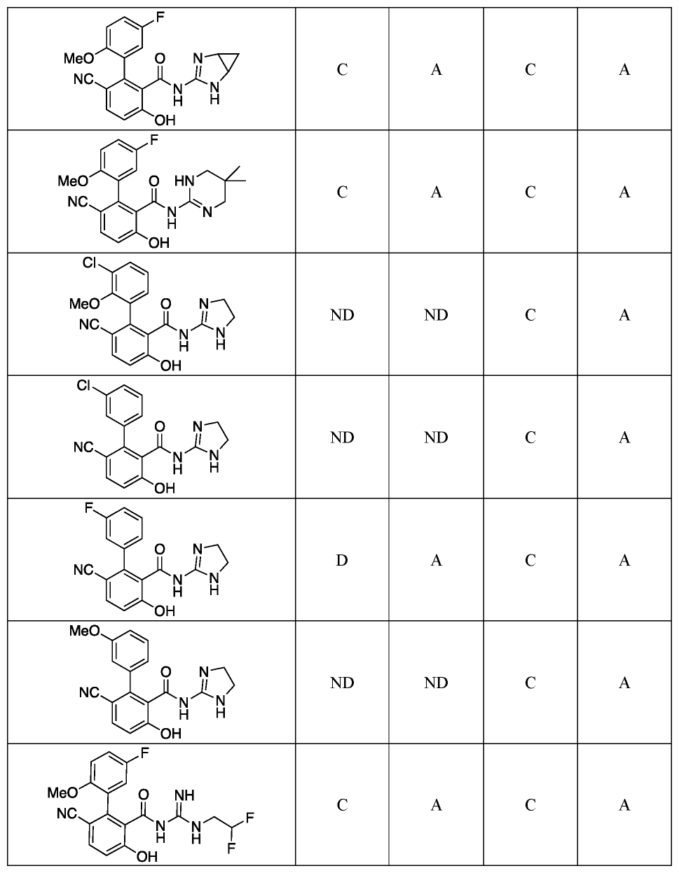

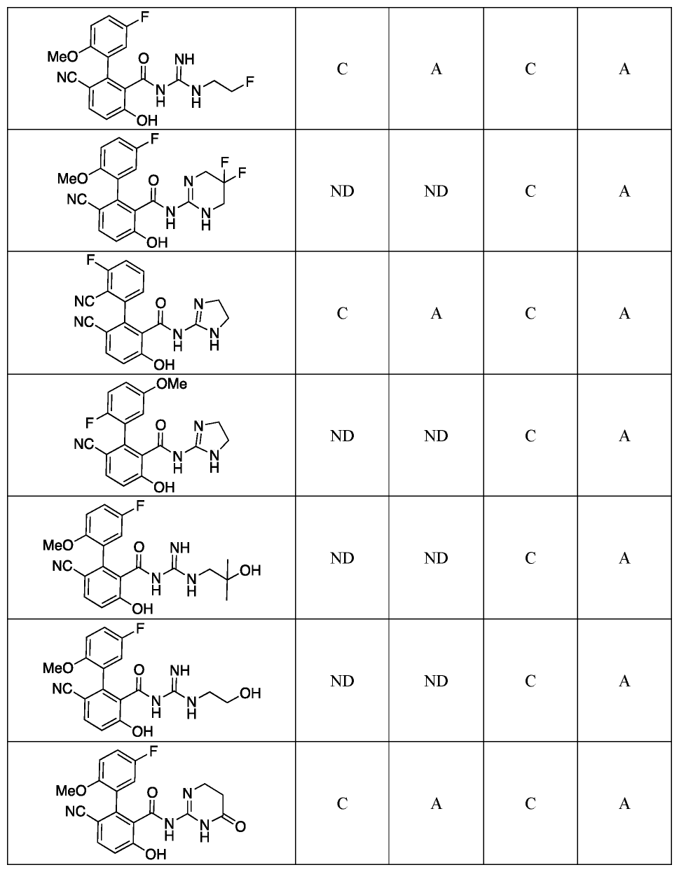

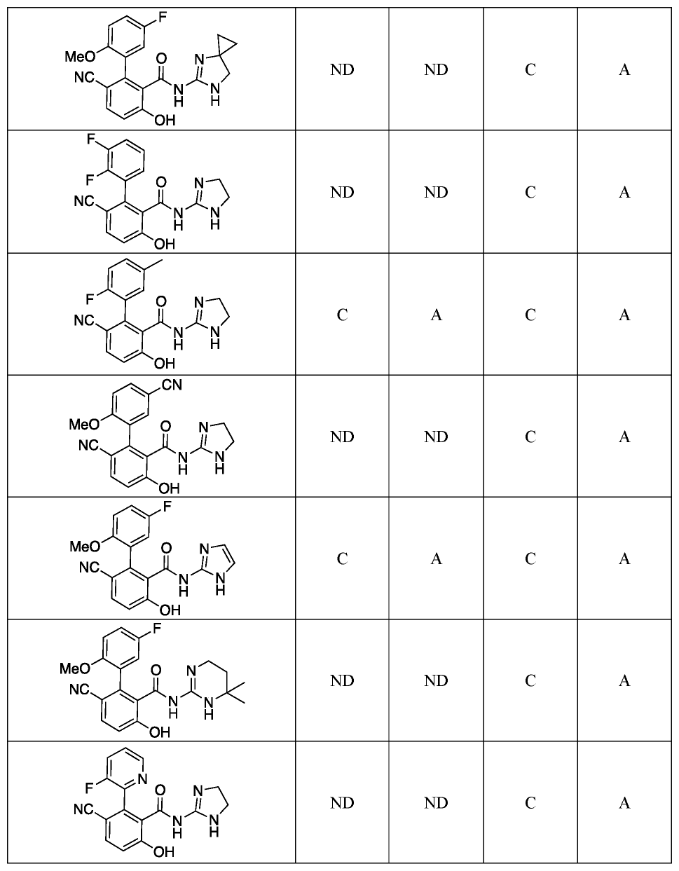

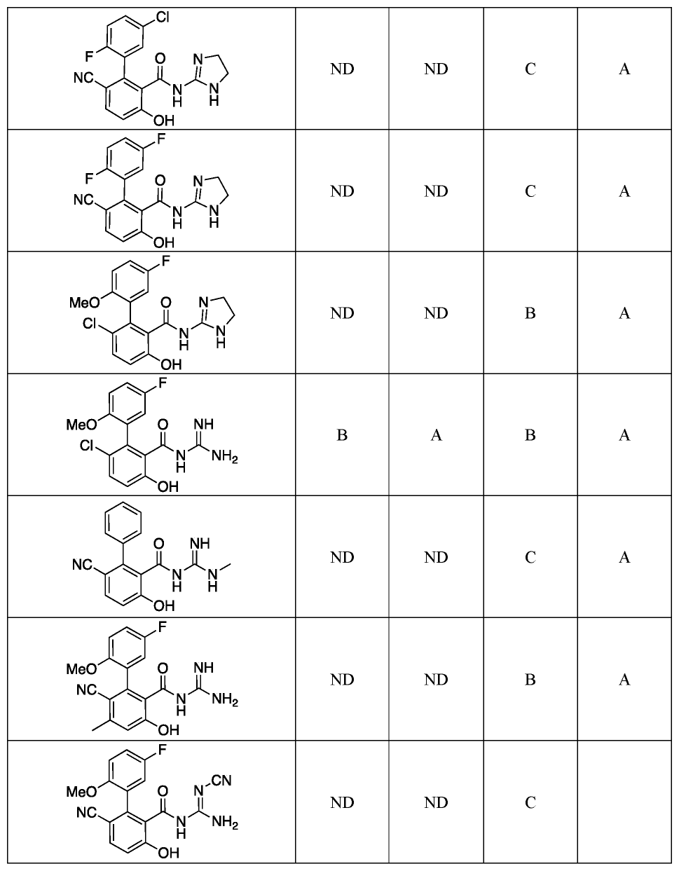

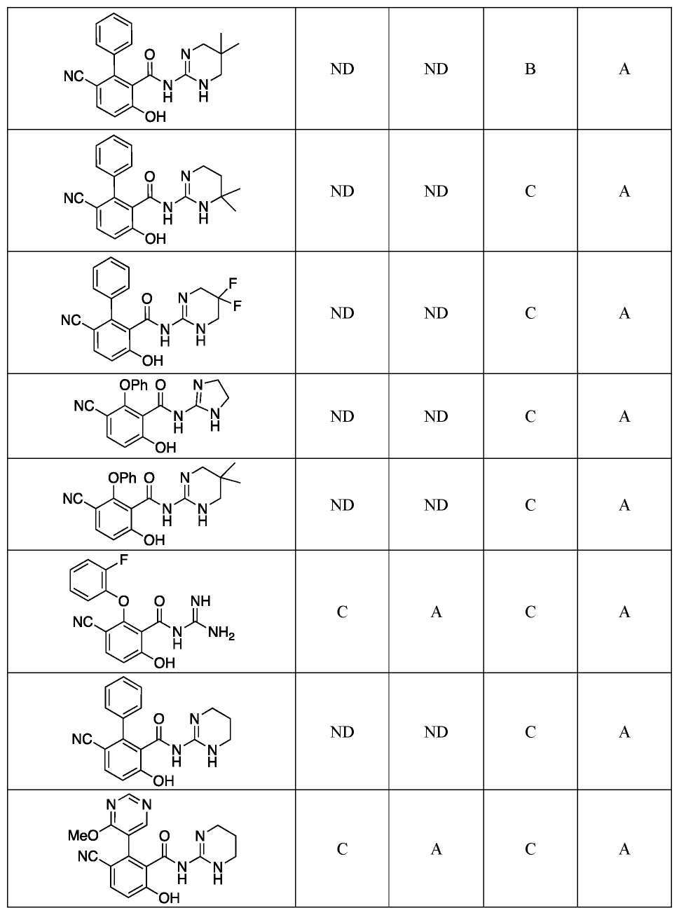

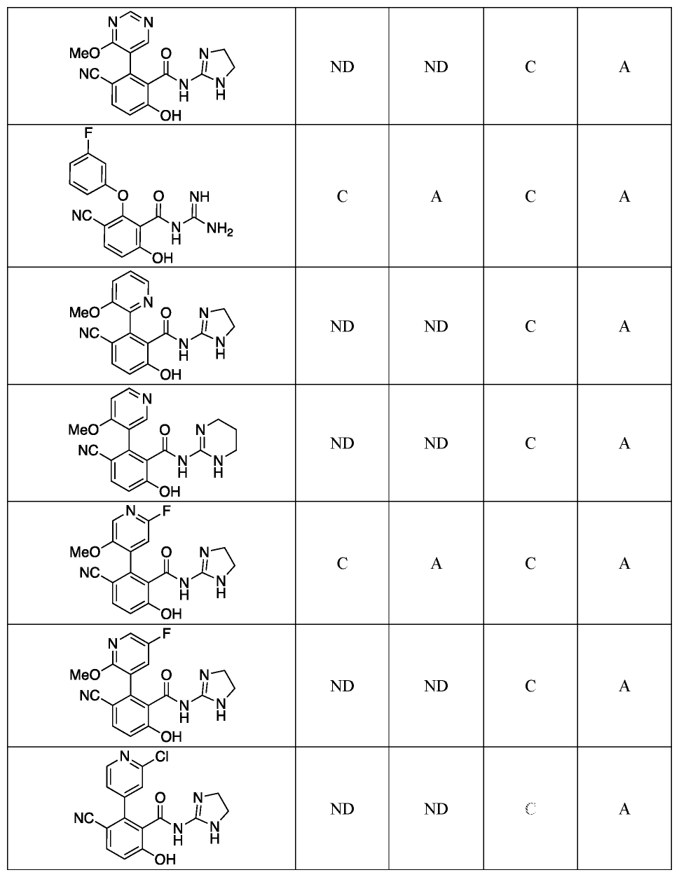

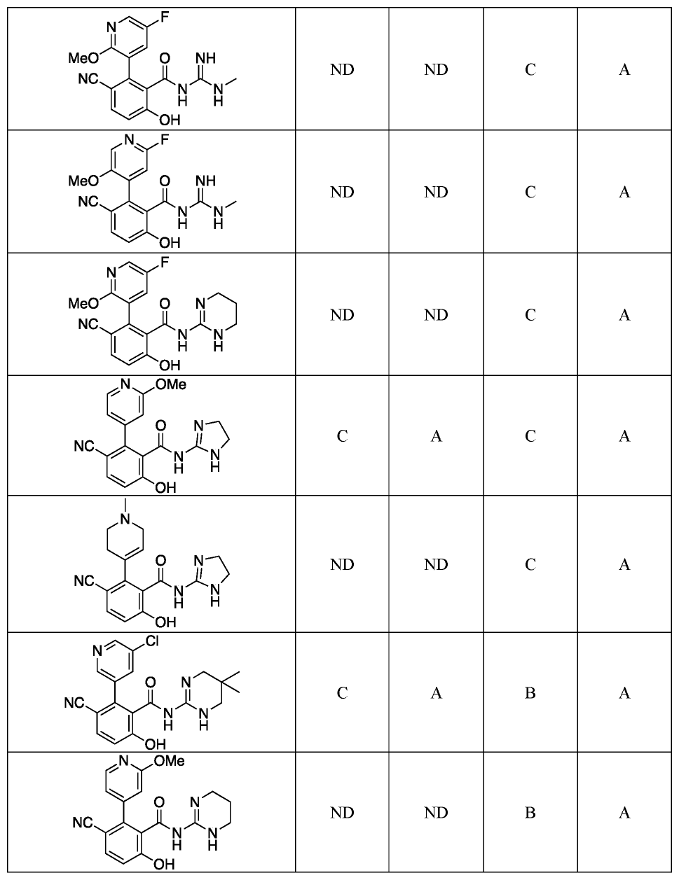

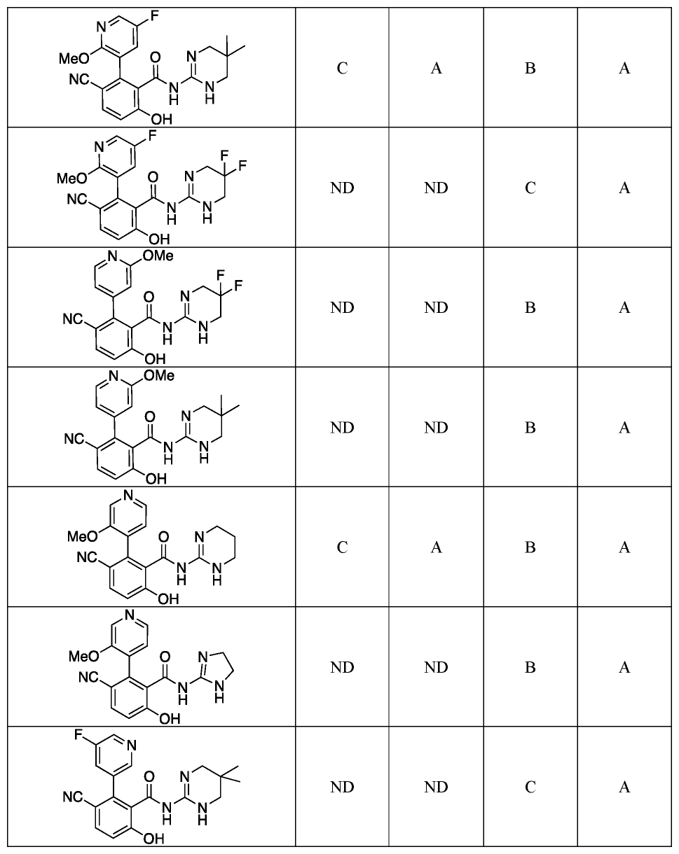

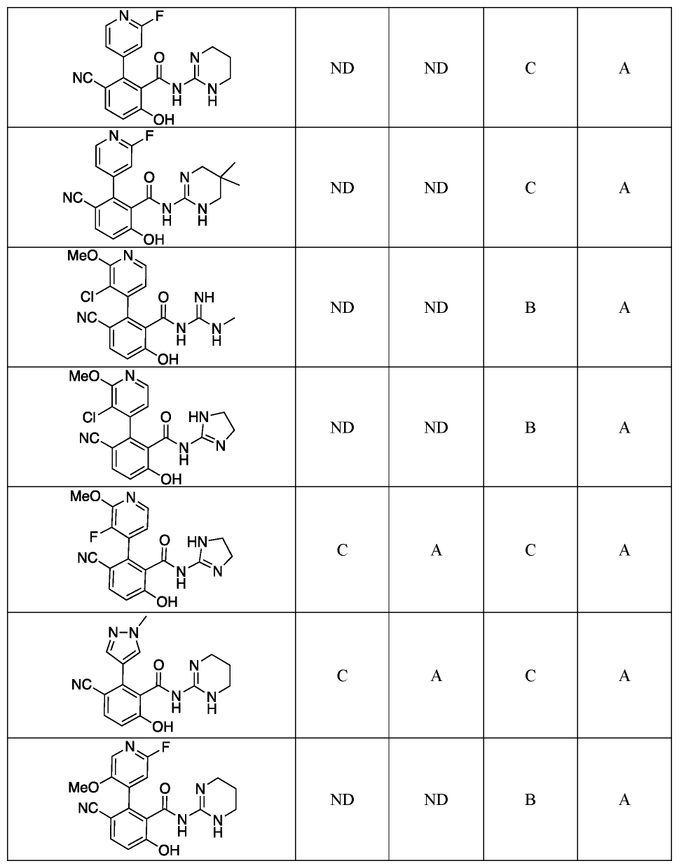

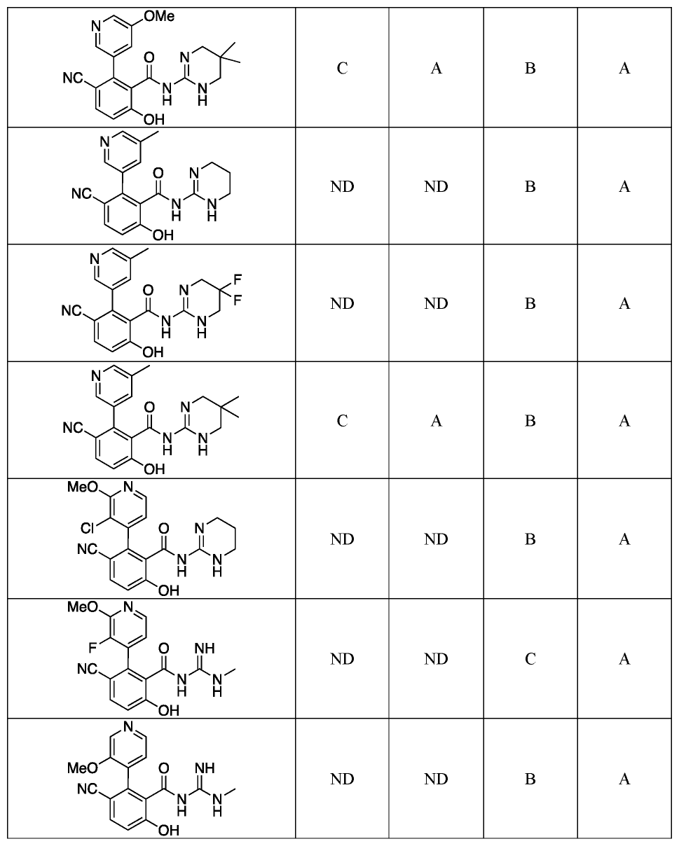

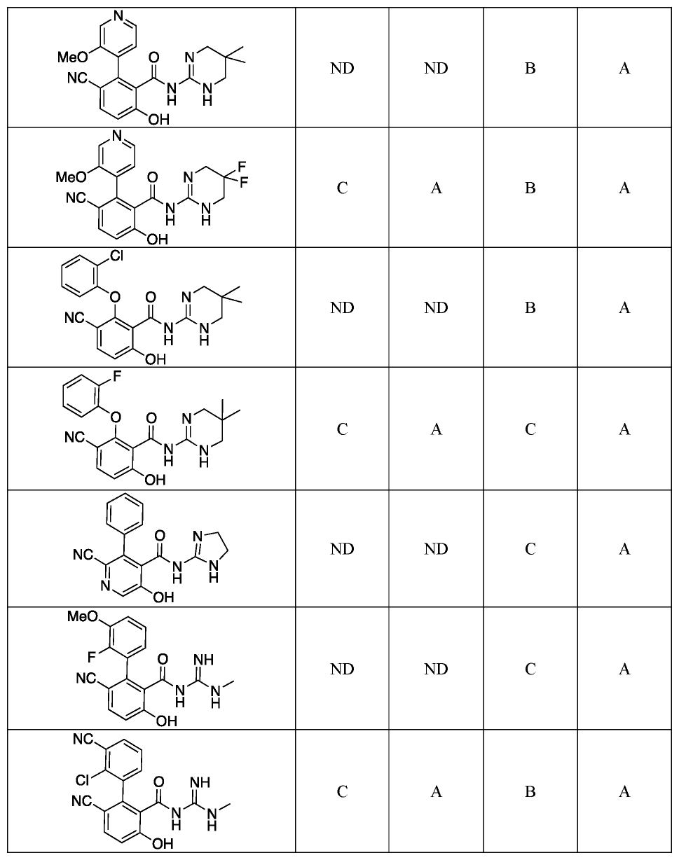

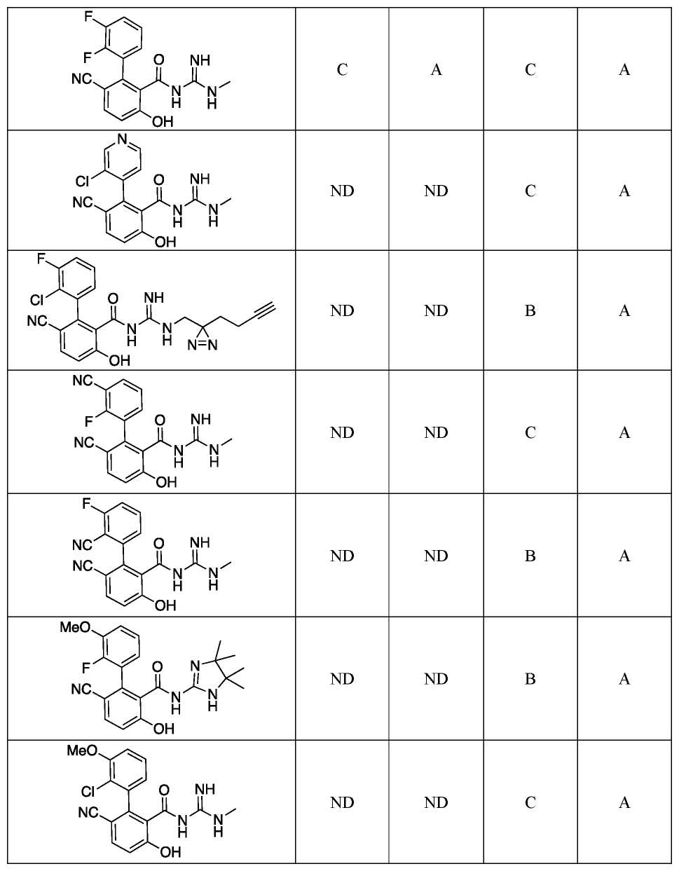

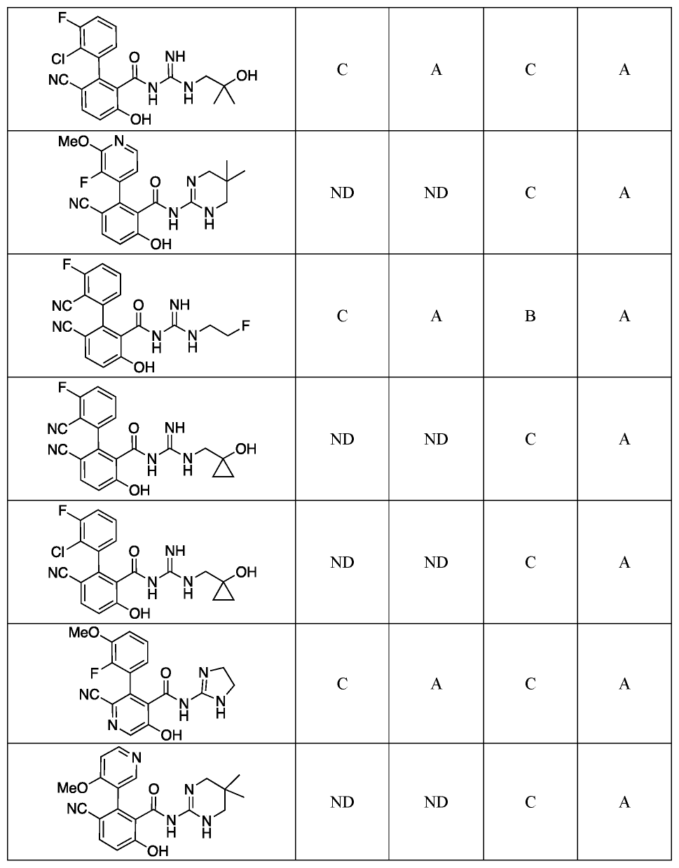

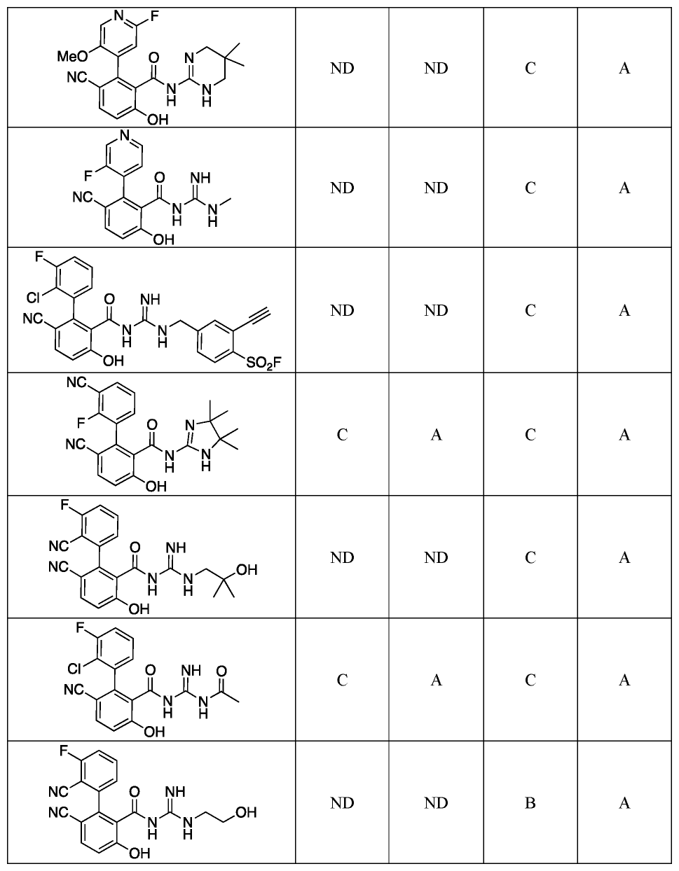

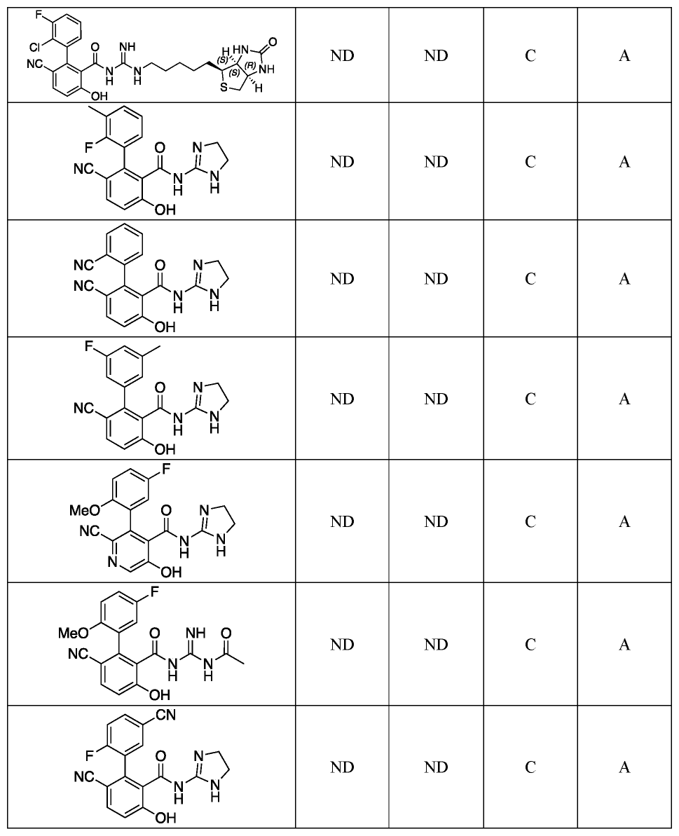

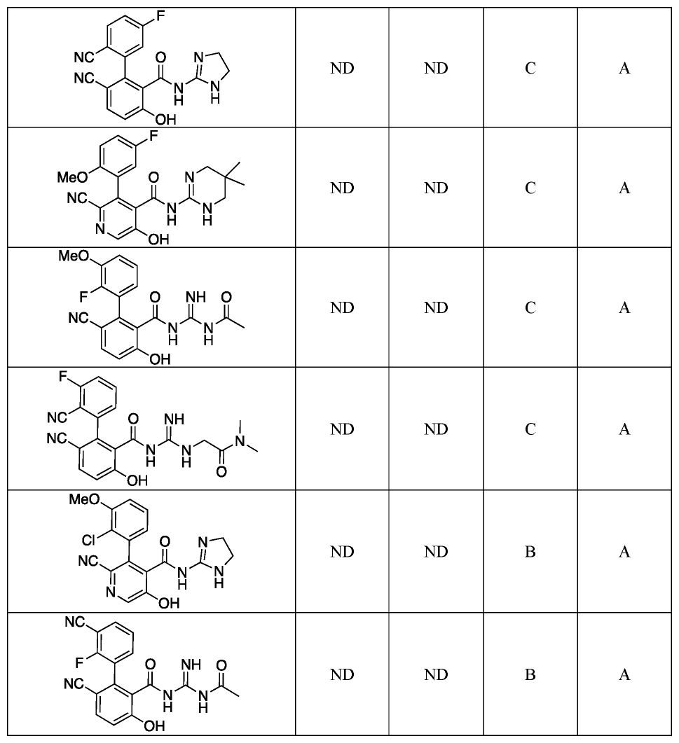

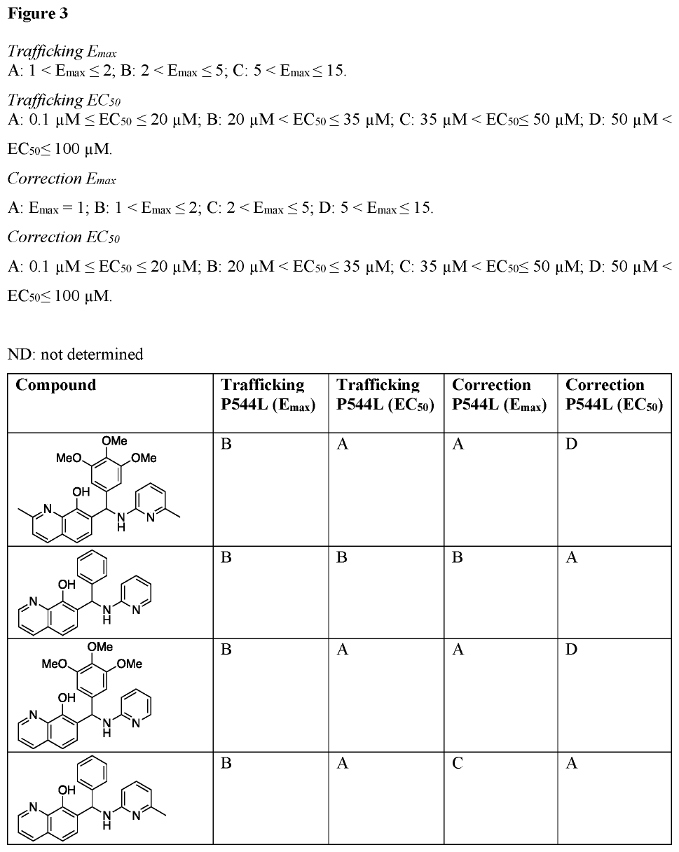

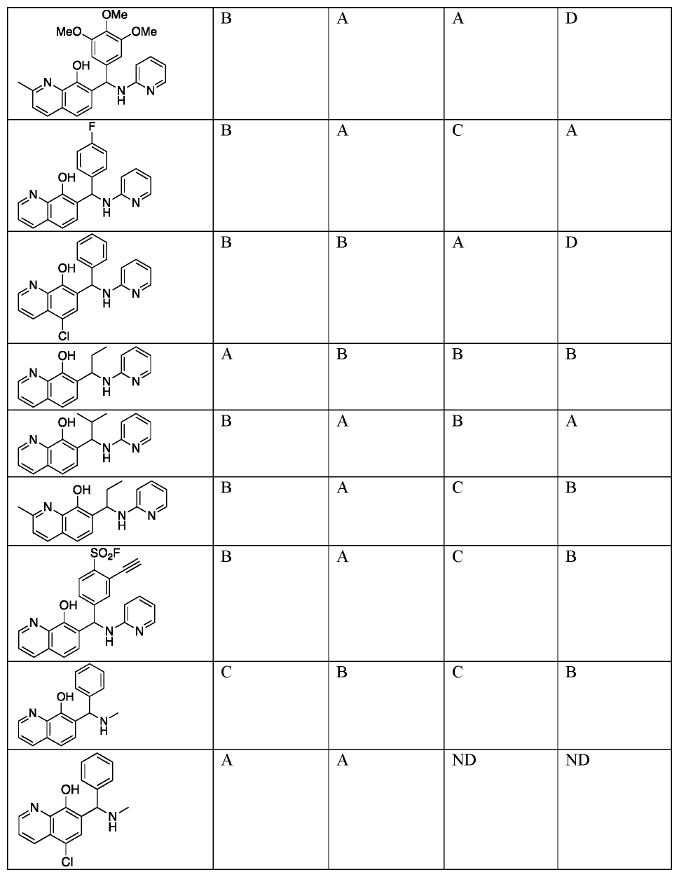

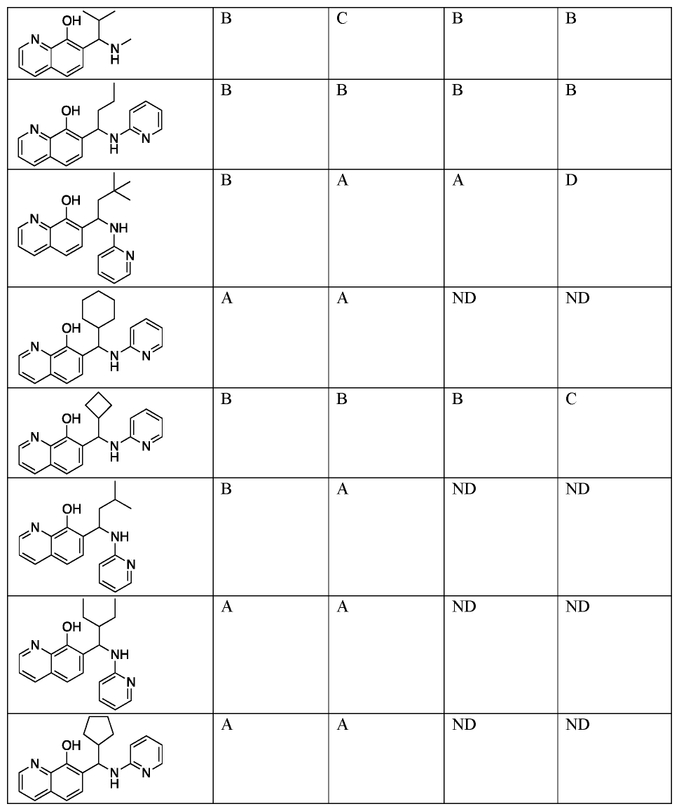

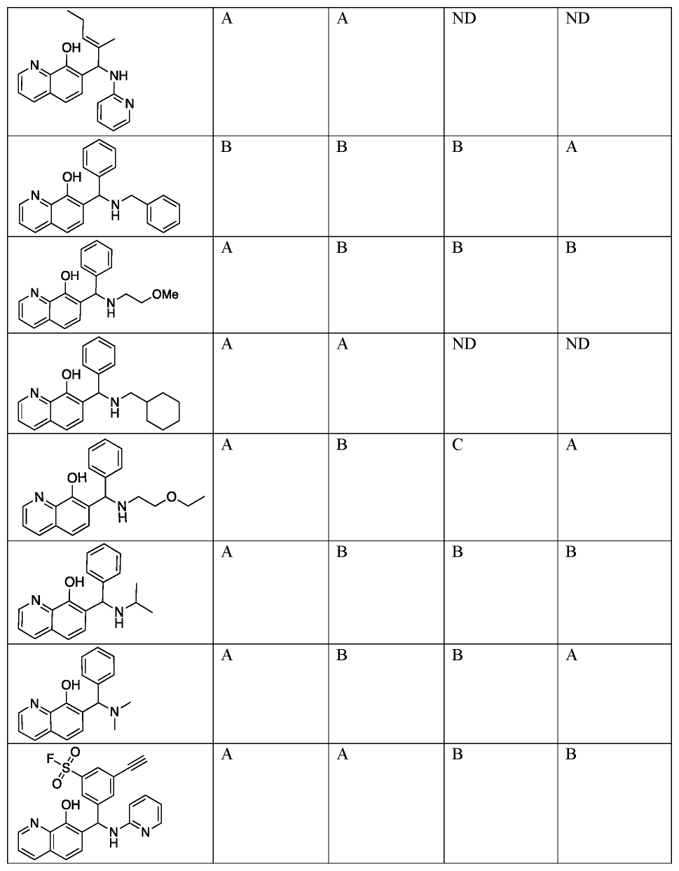

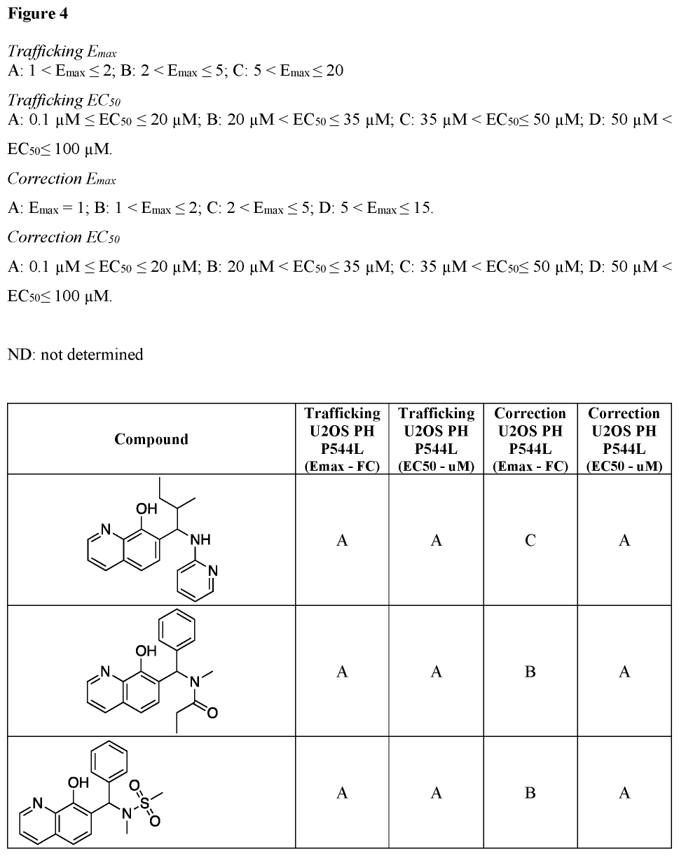

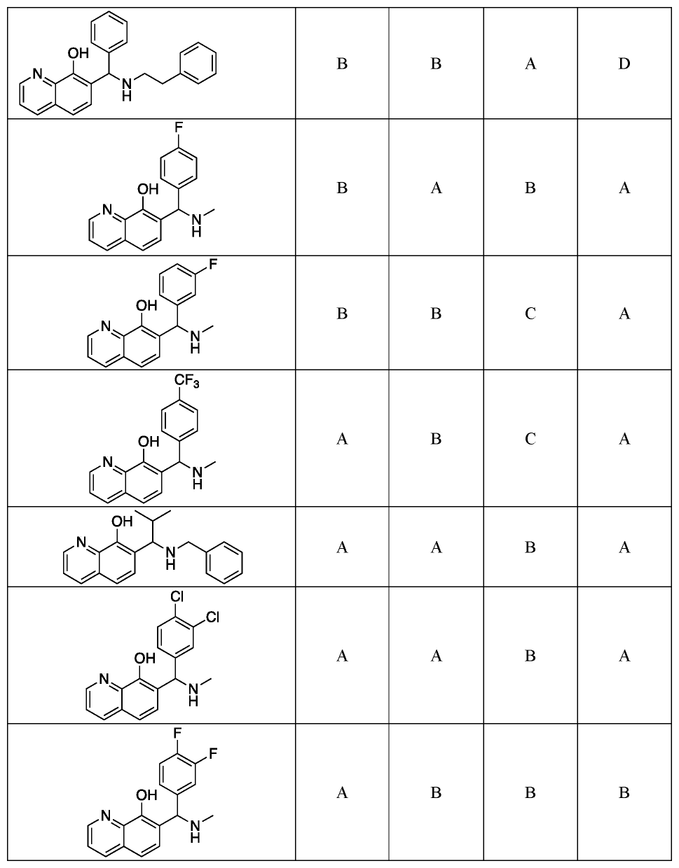

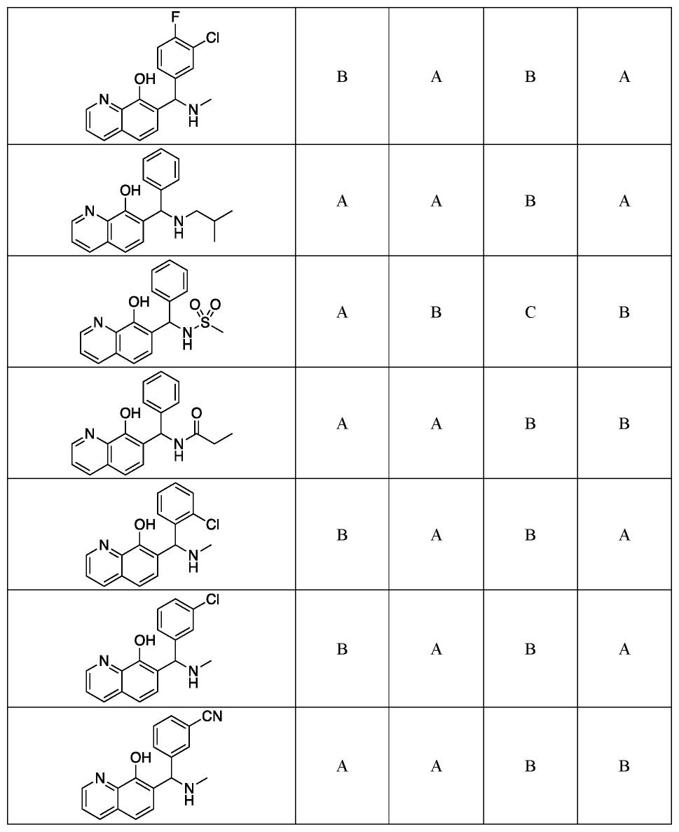

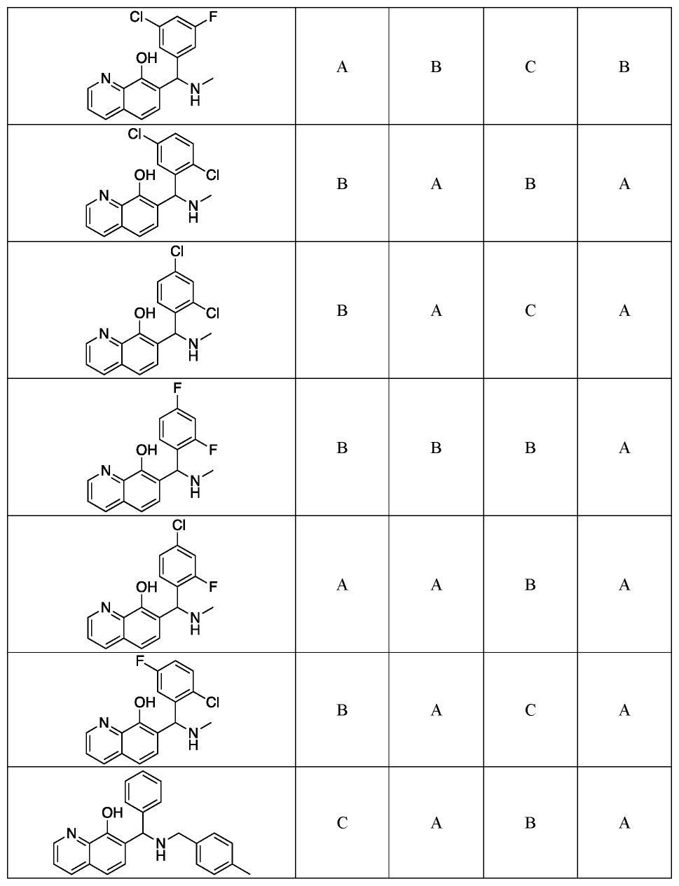

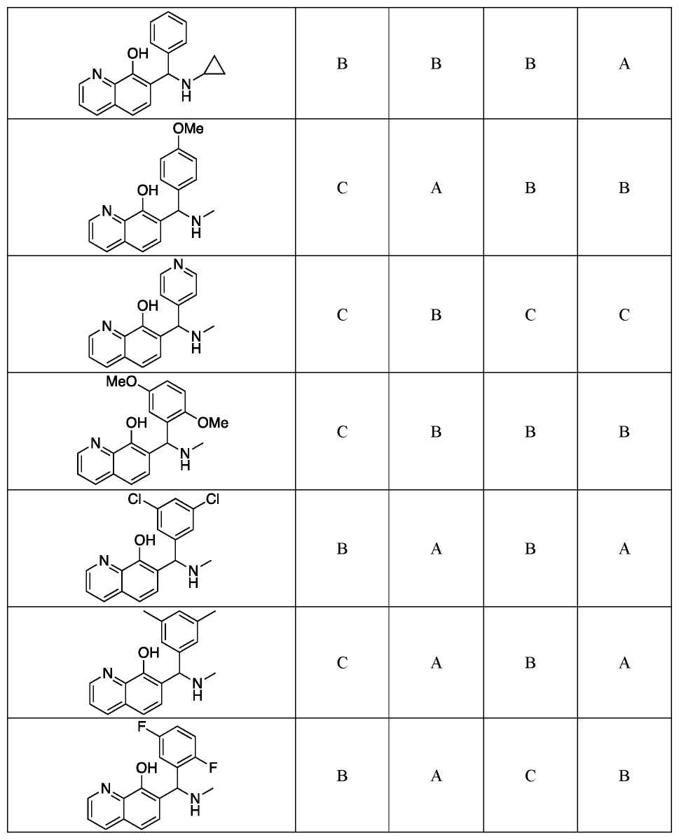

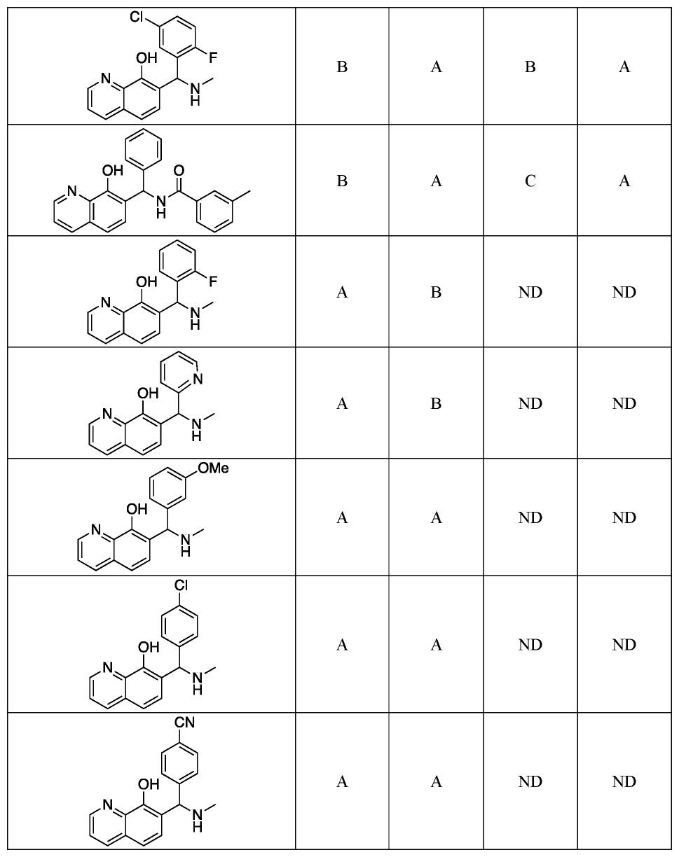

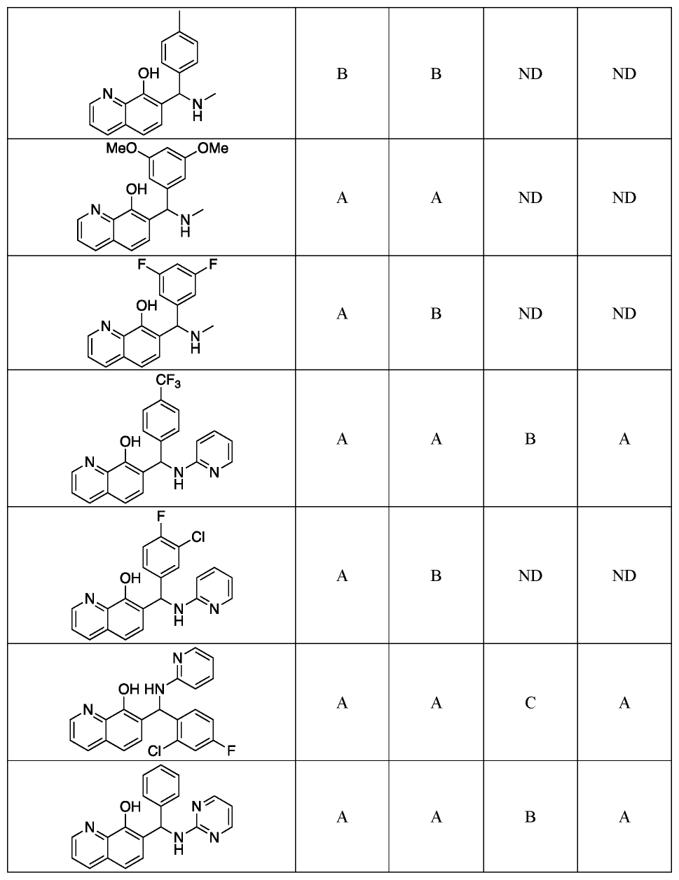

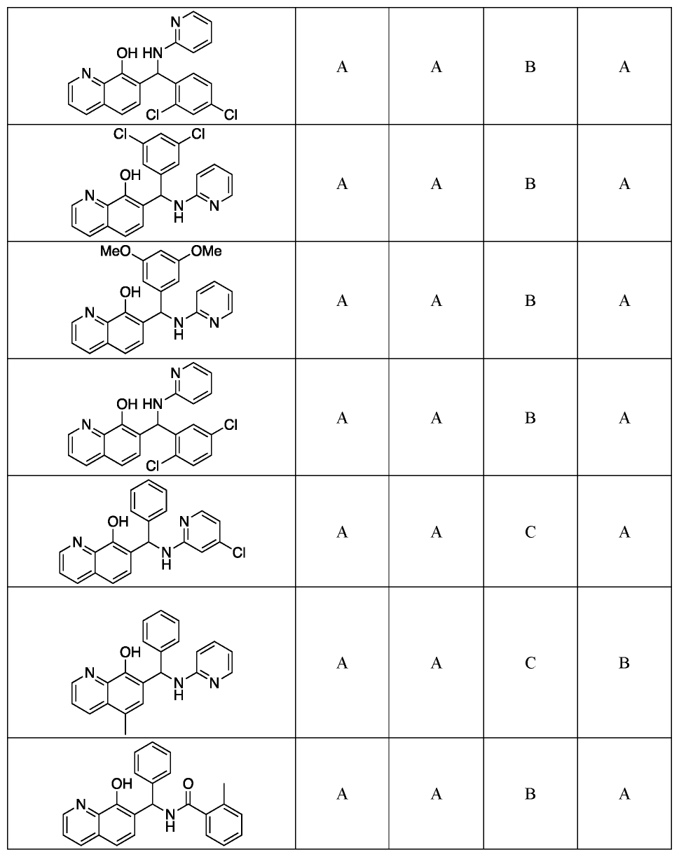

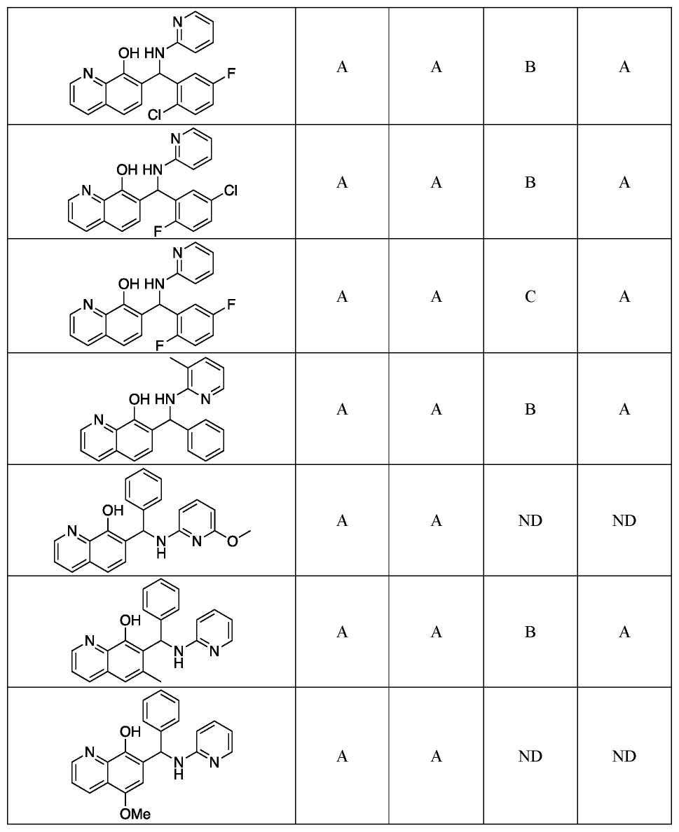

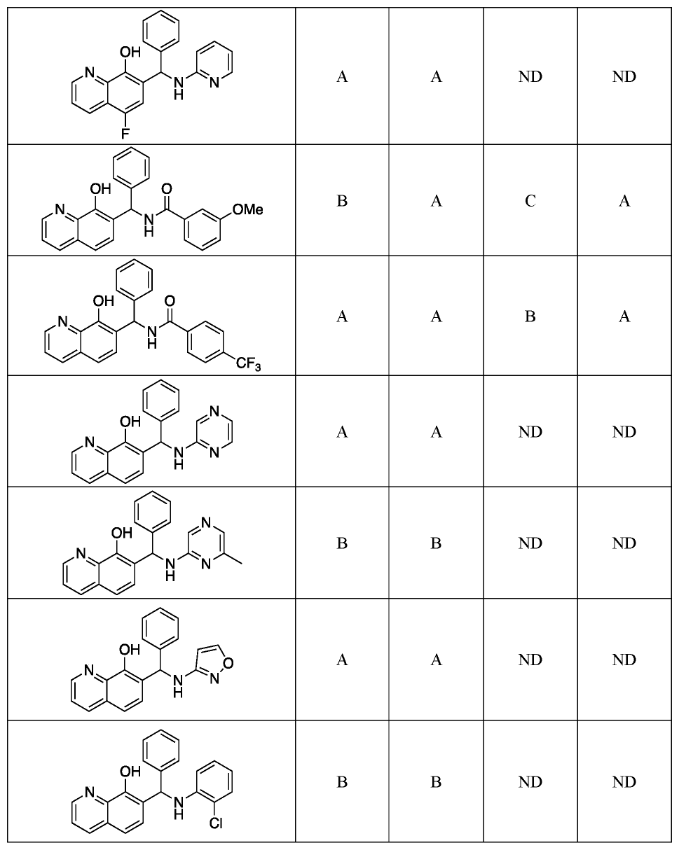

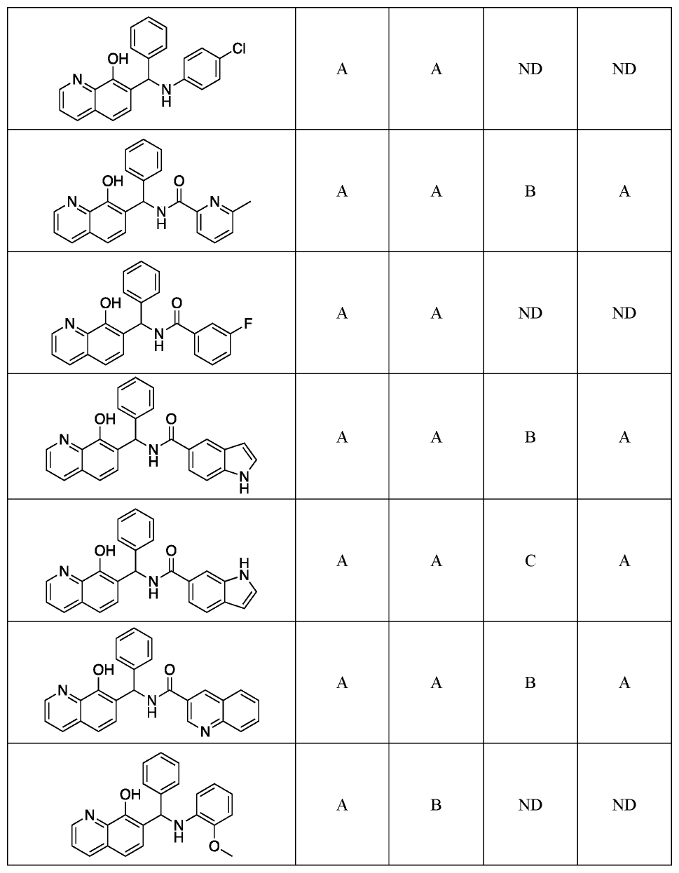

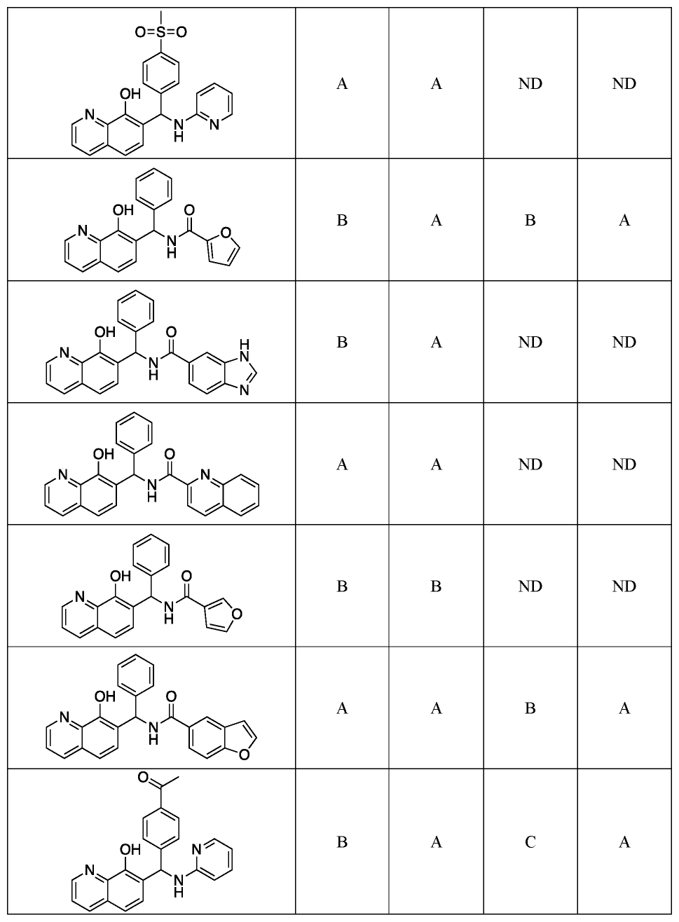

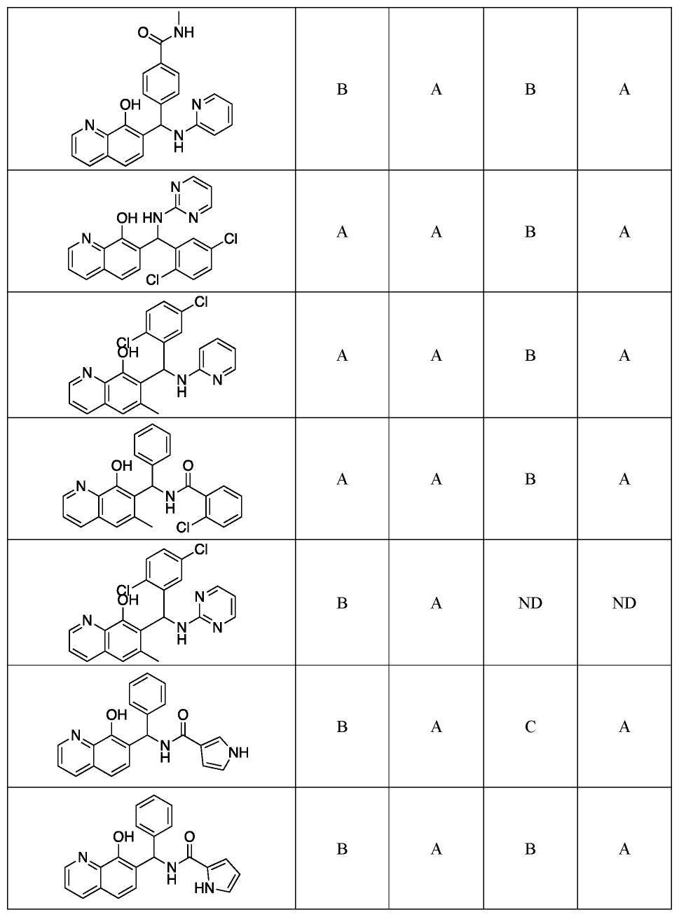

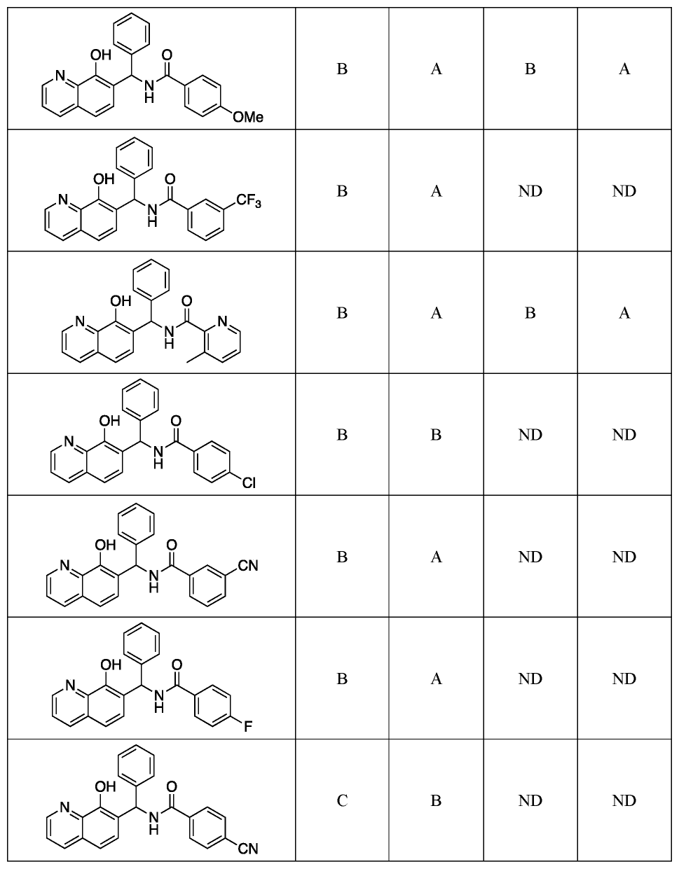

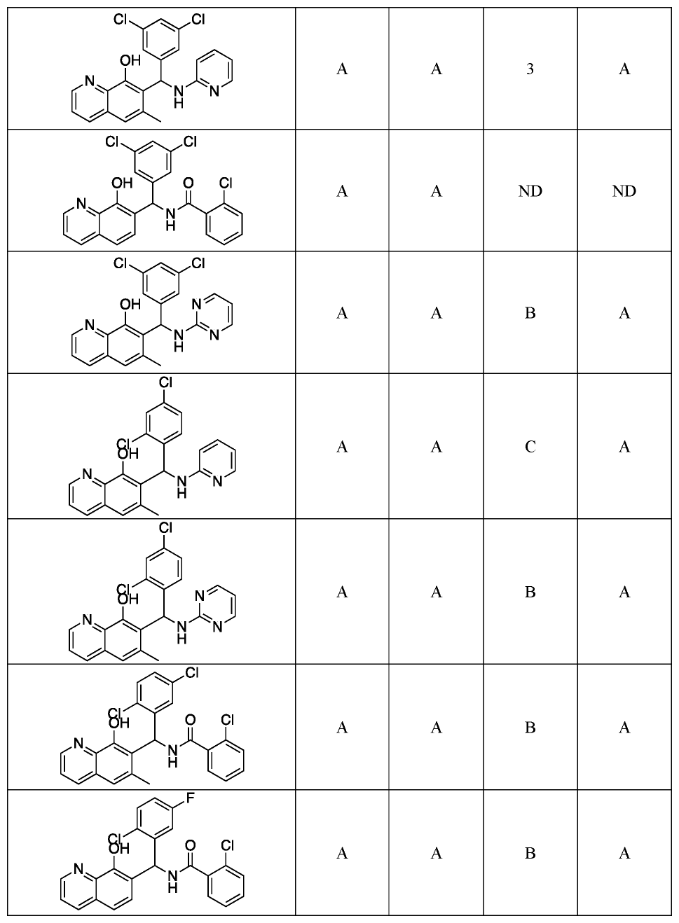

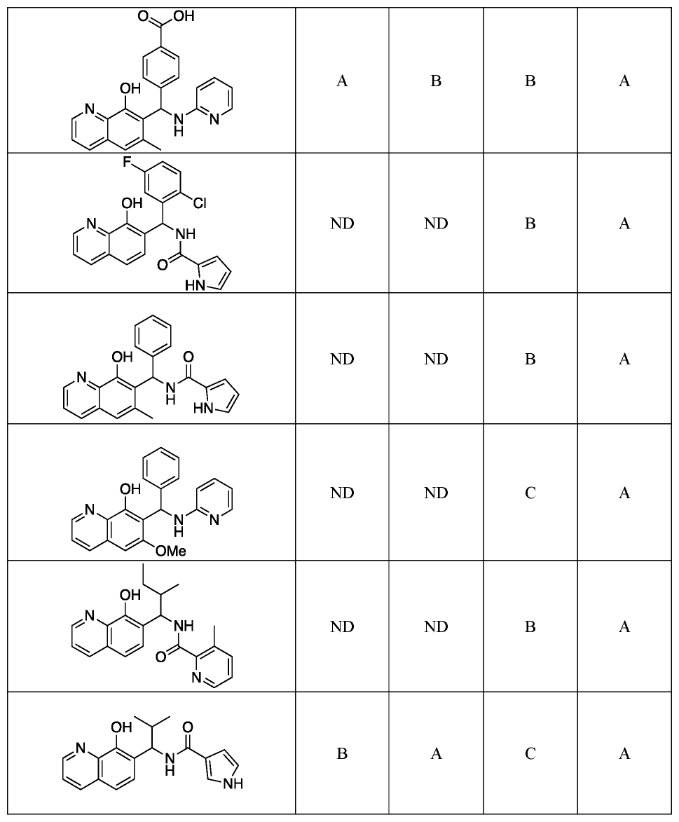

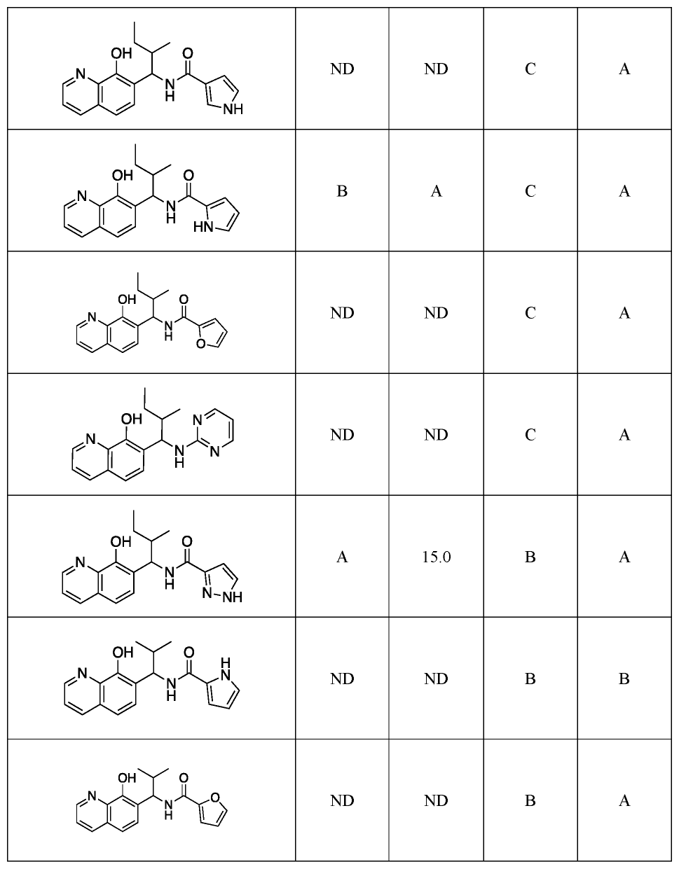

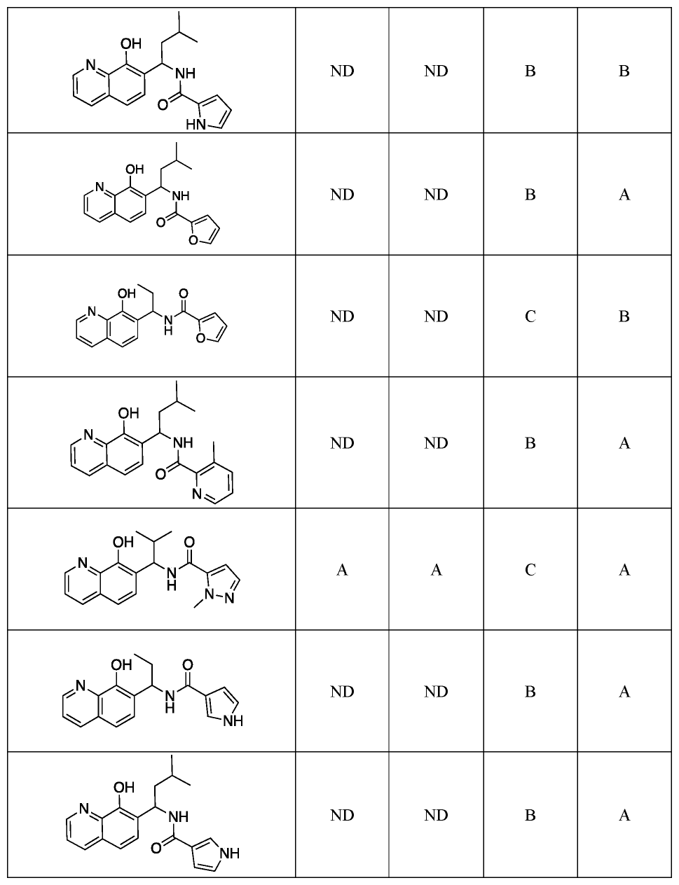

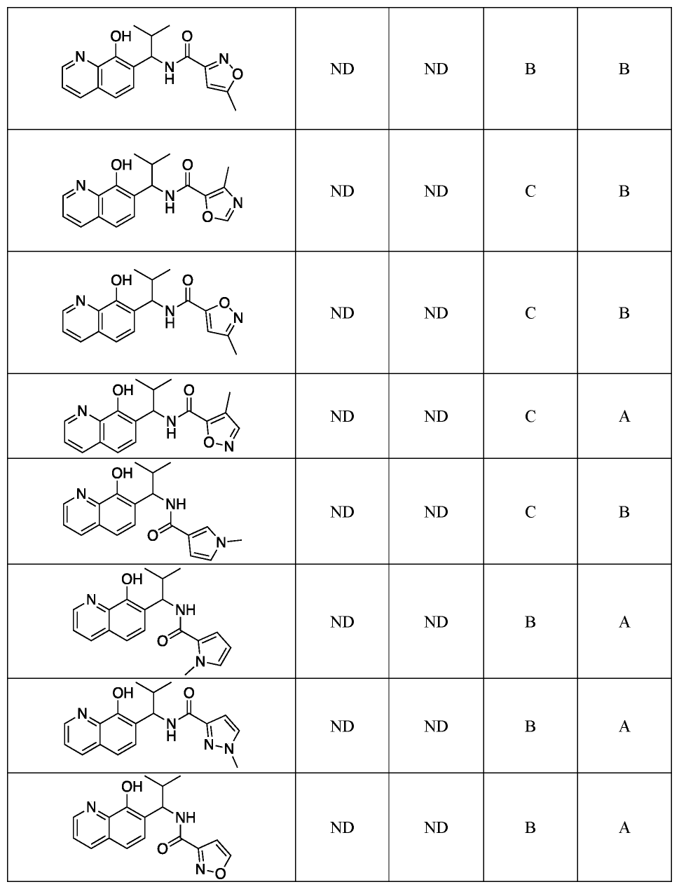

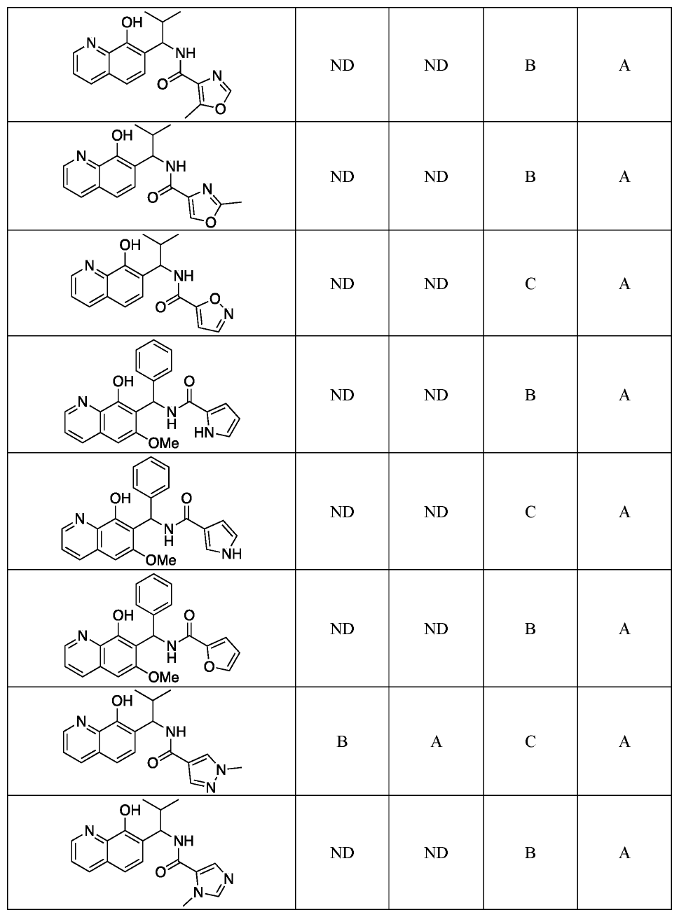

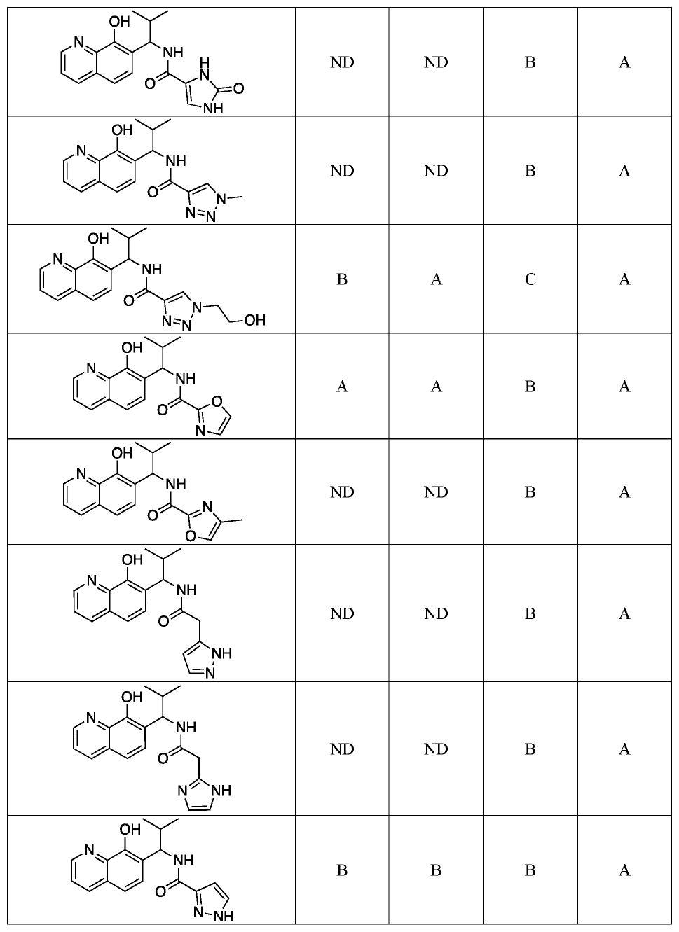

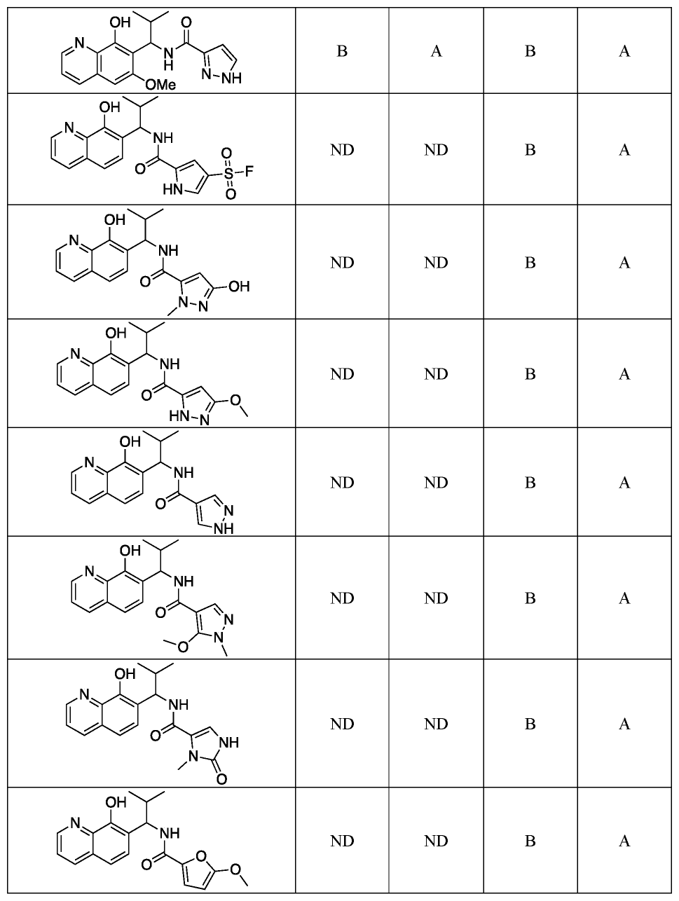

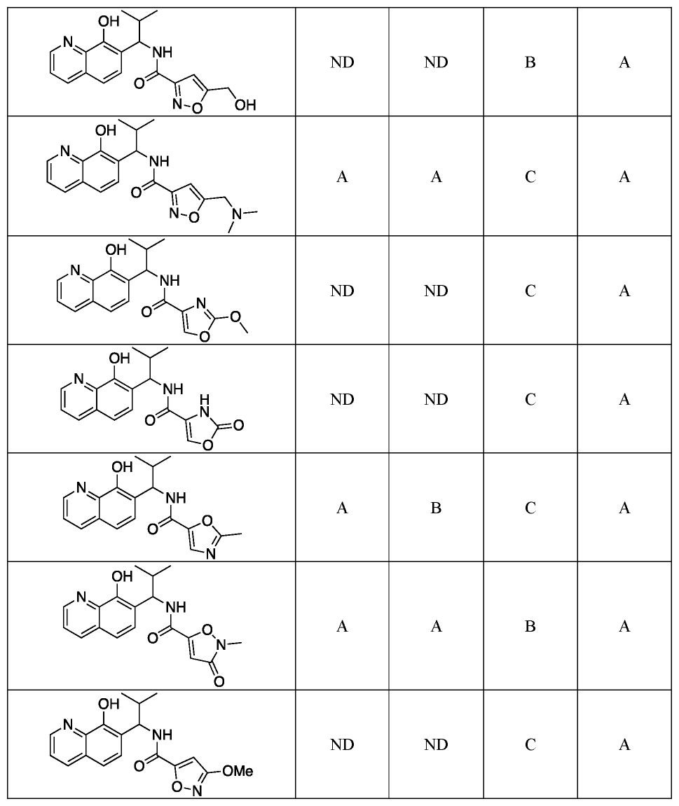

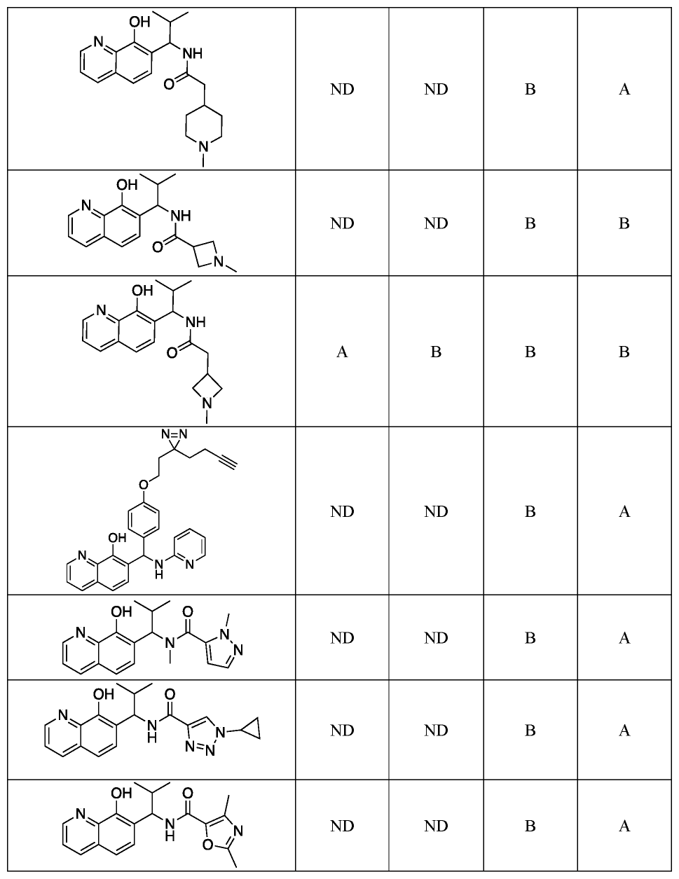

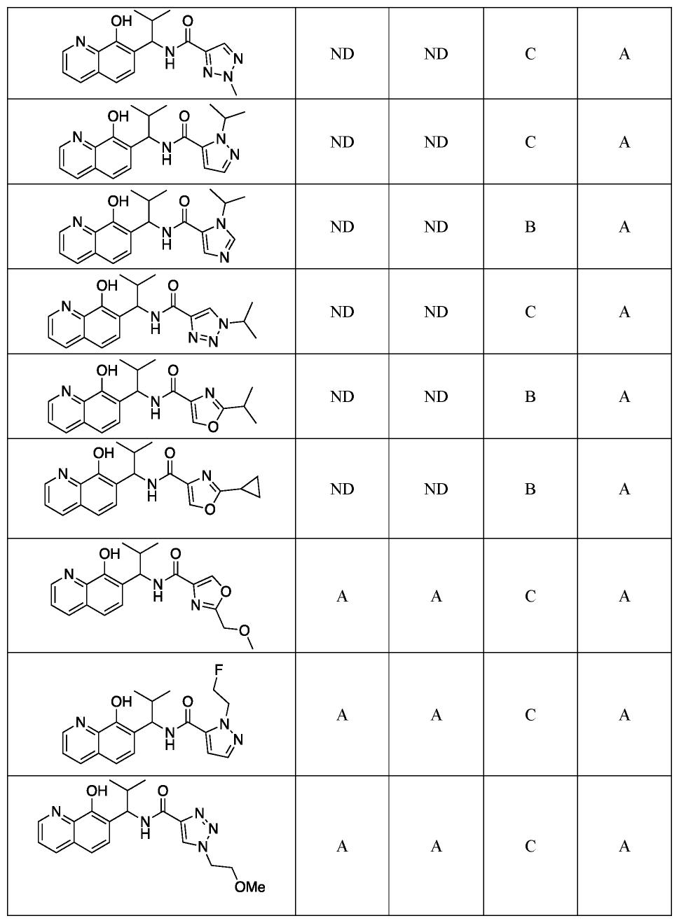

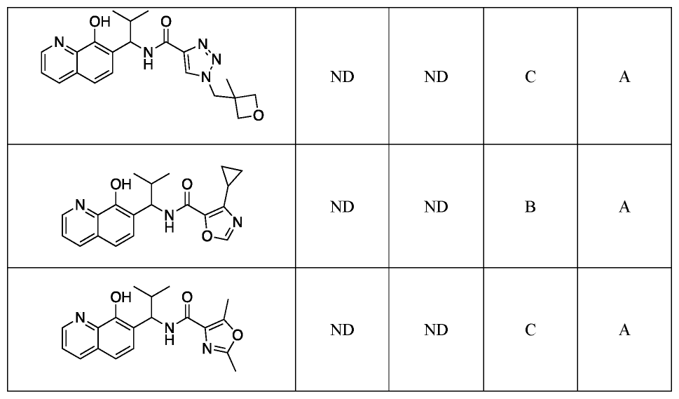

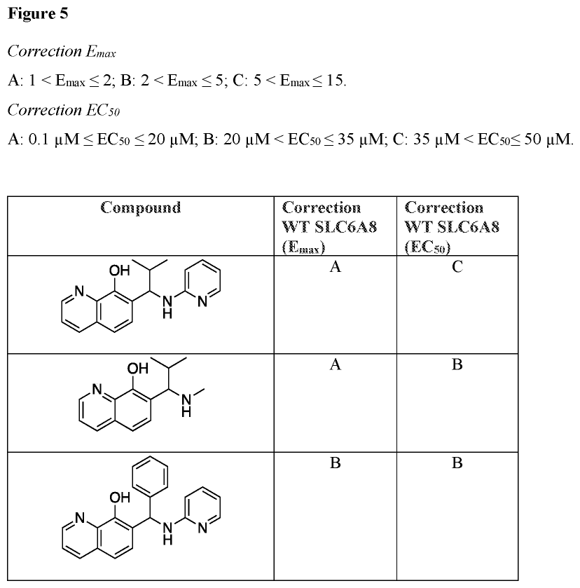

### DETAILED DESCRIPTION

### Definitions

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

The term “prodrug” is intended to encompass compounds which, under physiologic conditions, are converted into the therapeutically active agents of the present invention. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds of the invention in a formulation represented above can be replaced with the corresponding suitable prodrug, e.g., wherein a hydroxyl in the parent compound is presented as an ester or a carbonate or carboxylic acid present in the parent compound is presented as an ester.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da. about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

The term “pharmaceutically acceptable cocrystals” refers to solid coformers that do not form formal ionic interactions with the drug.

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

### SLC6A8

The SLC6A8 gene, located on the short arm of the sex chromosome, provides instructions for making a protein called sodium- and chloride-dependent creatine transporter 1. This protein transports the compound creatine into cells. Creatine is needed for the body to store and use energy properly.

At least 80 mutations in the SLC6A8 gene have been identified in people with X-linked creatine deficiency, a disorder that causes intellectual disability, behavioral problems, seizures, and muscle weakness. SLC6A8 gene mutations impair the ability of the transporter protein to bring creatine into cells, resulting in a creatine shortage (deficiency). The effects of creatine deficiency are most severe in organs and tissues that require large amounts of energy, especially the brain.

X-linked creatine deficiency is an inherited disorder that primarily affects the brain. People with this disorder have intellectual disability, which can range from mild to severe, and delayed speech development. Some affected individuals develop behavioral disorders such as attention deficit hyperactivity disorder or autistic behaviors that affect communication and social interaction. They may also experience seizures. Children with X-linked creatine deficiency may experience slow growth and exhibit delayed development of motor skills such as sitting and walking. Affected individuals tend to tire easily.

A small number of people with X-linked creatine deficiency have additional signs and symptoms including abnormal heart rhythms, an unusually small head (microcephaly), or distinctive facial features such as a broad forehead and a flat or sunken appearance of the middle of the face (midface hypoplasia).

### Methods of Treatment

Creatine Transporter Deficiency (CTD)

CTD is an inborn error of creatine metabolism in which creatine is not properly transported to the brain and muscles due to defective creatine transporters. CTD is an X-linked disorder caused by mutations in the SLC6A8 gene. The SLC6A8 gene is located on the short arm of the sex chromosome, Xq28. Hemizygous males with CID express speech and behavior abnormalities, intellectual disabilities, development delay, seizures, and autistic behavior. Heterozygous females with CTD generally express fewer, less severe symptoms. CTD is one of three different types of cerebral creatine deficiency (CCD). The other two types of CCD are guanidinoacetate methyltransferase (GAMT) deficiency and L-arginine:glycine amidinotransferase (AGAT) deficiency. Clinical presentation of CTD is similar to that of GAMT and AGAT deficiency. CID was first identified in 2001 with the presence of a hemizygous nonsense mutation in the SLC6A8 gene in a male patient.

CTD is difficult to treat because the actual transporter responsible for transporting creatine to the brain and muscles is defective. Studies in which oral creatine monohydrate supplements were given to patients with CID found that patients did not respond to treatment. However, similar studies conducted in which patients that had GAMT or AGAT deficiency were given oral creatine monohydrate supplements found that patient's clinical symptoms improved. Patients with CTD are unresponsive to oral creatine monohydrate supplements because regardless of the amount of creatine they ingest, the creatine transporter is still defective, and therefore creatine is incapable of being transported across the BBB.

Accordingly, in certain embodiments, the invention provides methods of treating creatine transporter deficiency, comprising administering to a mammal in need thereof a therapeutically effective amount of a compound that increases transport of a substrate by a creatine transporter or protein.

In some embodiments, the substrate is creatine or a salt thereof. In some embodiments, the substrate is guanidinoacetic acid or a salt thereof. In some embodiments, the substrate is 3-guanidinopropionic acid or a salt thereof. In some embodiments, the substrate is 4-guanidinobutyric acid or a salt thereof. In some embodiments, the substrate is guanidinoethane sulfonic acid or a salt thereof.

In some embodiments, the creatine transporter is a mutant creatine transporter. In some embodiments, the mutant creatine transporter is mutant SLC6A8.

In some embodiments, the compound increases transport of the substrate into an endothelial cell. In some embodiments, the endothelial cell is a brain endothelial cell. In some embodiments, the compound increases transport of the substrate into a gut epithelial cell, a brain cell, or a muscle cell. In some embodiments, the compound increases transport of the substrate into a gut epithelial cell. In some embodiments, the compound increases transport of the substrate into a brain cell. In some embodiments, the compound increases transport of the substrate into a muscle cell.

One aspect of the invention relates to methods of treating creatine transporter deficiency, comprising administering to a mammal in need thereof a therapeutically effective amount of a compound that increases transport of a substrate across the blood-brain barrier by a creatine transporter or protein.

In some embodiments, the substrate is creatine or a salt thereof. In some embodiments, the substrate is guanidinoacetic acid or a salt thereof. In some embodiments, the substrate is 3-guanidinopropionic acid or a salt thereof. In some embodiments, the substrate is 4-guanidinobutyric acid or a salt thereof. In some embodiments, the substrate is guanidinoethane sulfonic acid or a salt thereof.

In some embodiments, wherein the creatine transporter is a mutant creatine transporter. In some embodiments, the mutant creatine transporter is mutant SLC6A8.

One aspect of the invention relates to methods of treating creatine transporter deficiency, comprising administering to a mammal in need thereof a therapeutically effective amount of a compound that increases transport of a substrate across the neuronal plasma membrane by a creatine transporter or protein.

In some embodiments, substrate is creatine or a salt thereof. In some embodiments, the substrate is guanidinoacetic acid or a salt thereof. In some embodiments, the substrate is 3-guanidinopropionic acid or a salt thereof. In some embodiments, the substrate is 4-guanidinobutyric acid or a salt thereof. In some embodiments, the substrate is guanidinoethane sulfonic acid or a salt thereof.

In some embodiments, the creatine transporter is a mutant creatine transporter. In some embodiments, the mutant creatine transporter is mutant SLC6A8.

Another aspect of the invention relates to methods of decreasing accumulation or the concentration of guanidinoacetic acid or a salt thereof in a cell, comprising administering to a mammal in need thereof a therapeutically effective amount of a compound that increases transport of guanidinoacetic acid or a salt thereof by a creatine transporter or protein.

In some embodiments, the compound decreases intracellular accumulation of guanidinoacetic acid or a salt thereof. In some embodiments, wherein the compound decreases the intracellular concentration of guanidinoacetic acid or a salt thereof.

In some embodiments, the creatine transporter is a mutant creatine transporter. In some embodiments, the mutant creatine transporter is mutant SLC6A8.

In some embodiments, the cell is a brain cell.

One aspect of the invention relates to methods of increasing transport of guanidinoacetic acid or a salt thereof across the blood-brain barrier, comprising administering to a mammal in need thereof a therapeutically effective amount of a compound that increases transport of guanidinoacetic acid or a salt thereof by a creatine transporter or protein.

In some embodiments, the creatine transporter is a mutant creatine transporter. In some embodiments, the mutant creatine transporter is mutant SLC6A8.

Subjects to be Treated

In one aspect of the invention, a mammal is selected on the basis that it presents with CTD.

In CTD symptoms can significantly vary between hemizygous males and heterozygous females, although, symptoms are generally more severe in hemizygous males. Hemizygous males more commonly express seizures, growth deficiency, severe mental retardation, and severe expressive language impairment.

The methods are effective for a variety of mammals, e.g., humans and other animals, such as laboratory animals, e.g., mice, rats, rabbits, or monkeys, or domesticated and farm animals, e.g., cats, dogs, goats, sheep, pigs, cows, or horses. In some embodiments, the mammal is a male. In some embodiments, the mammal is a female. In some embodiments, the mammal is a primate, equine, bovine, feline, or canine. In some embodiments, the mammal is a human.

Pharmaceutical Compositions, Routes of Administration, and Dosing

In certain embodiments, the invention is directed to a pharmaceutical composition, comprising a compound of the invention and a pharmaceutically acceptable carrier. In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of ischemia-reperfusion injury.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt or cocrystal thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Phannaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (al-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (μm), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The invention is further described in the following examples, which do not limit the scope of the invention described in the claims.

### Example 1: PathHunter MEM-EA Pharmacotrafficking Assay for SLC6A8 CTD Mutants Cell Lines

### Preparation of Cells

U-2 OS MEM-EA cells were purchased from Eurofins (catalog #93-1101C3). From these parental cells, stable cell lines expressing SLC6A8 CTD mutants were made using standard cell culture protocols, involving transfections of plasmids followed by antibiotic selection. These plasmids encoded CTD mutant SLC6A8 proteins with a C-terminal ProLink2 tag. U-2 OS MEM-EA cells and derived stable cell lines were grown in RPMI medium 1640 (Thermo Fisher Scientific, catalog #A10491-01) supplemented with 10% Fetal Bovine Serum (FBS), 200 ug/mL hygromycin B (Thermo Fisher Scientific, catalog #10687010), 100 mg/mL streptomycin, and 100 U/mL penicillin. Cells were grown at 37° C. in a humidified CO₂ incubator.

### Assay

U-2 OS MEM-EA cells stably expressing SLC6A8 CTD mutants were plated into white-walled 96-well plates (Corning, catalog #3903) at a density of 20,000 cells per well. For background subtraction, the parental U-2 OS MEM-EA cells were also plated. After 24 hrs, compounds were dispensed directly into the plated cells using the Tecan D300e Digital Dispenser. After an additional 24 hrs, the media with compound was again removed and white covers (Thermo Fisher Scientific, catalog #236272) were placed on the bottoms of the 96-well plates. Luminescence indicative of SLC6A8 CTD mutant cell surface localization was measured according to the manufacturer's protocol, using the PathHunter Detection kit (Eurofins catalog #93-0001L) and an EnVision plate reader (PerkinElmer, 2104 multilabel reader). Data were analyzed in Excel. Background signal from wells containing parental cells was subtracted, and then fold-changes were computed with respect to DMSO.

### Example 2: Corrector Assay for SLC6A8 CTD Mutant Cell Lines

### Preparation of Cells

A number of SLC6A8 CTD mutant cell lines were made in U-2 OS MEM-EA cells, 293T cells, HeLa cells, and CHO cells. All cells lines were generated as described above for U-2 OS MEM-EA cells, namely stable cell lines expressing SLC6A8 CTD mutants were made using standard cell culture protocols involving transfections of plasmids followed by antibiotic selection.

### Assay

Stable cell lines expressing CTD mutants were plated into 96-well plates (Corning, catalog #3595) at a density of 40,000 cells per well. After 24 hrs, compounds were dispensed directly into the plated cells using a Tecan D300e Digital Dispenser.

After an additional 24 hrs, the media with compound was removed. Cells were then incubated with a solution of 100 uM D3-creatine (SIGMA, 616249-1G) in media (without FBS) This solution was incubated with the cells for a 30 min incubation at 37° C. After the incubation, the media was removed, and the cells were washed once with 180 uL of phosphate buffered solution (PBS). To extract metabolites, water was added to the cells for 1 hour with vigorous shaking at 700 rpm. Cell extracts were analyzed on an ABSciex-4000 triple quad mass spectrometer coupled with a RapidFire sample desalting/injection system with a graphitic carbon desalting column and a basic buffer system in reverse phase. Abundances of D3-creatine were analyzed in Excel, and then fold-changes were computed with respect to DMSO.

### Example 3: General Procedures for the Synthesis of Representative Compounds of the Invention

### Synthesis of Acylguanidines

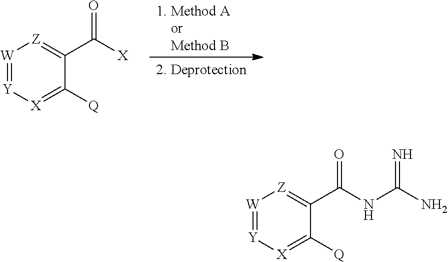 [0121] When Q=OH it might be protected with MOM or Benzyl group

### General Procedure A

Step 1—When X═OMe; Method A: guanidine hydrochloride, t-BuOK, DMF, RT
When X═OH; Method B: Boc-guanidine, NMM, BOP, DMF, RT

### Step 2

When Q=OMOM and guanidine is Boc-protected—TFA, DCM, RT
When Q=OBn and guanidine is Boc-protected—H₂, Pd/C, EtOAc, RT, then TFA, DCM, RT

### Step 1—Method A

To a suspension of guanidine hydrochloride (10 equiv.) in dry DMF (0.5M) t-BuOK (8 equiv.) was added under argon atmosphere. The resulting mixture was stirred at RT for 45 min, then a solution of the appropriate ester intermediate (1 equiv.) in dry DMF (0.8M) was added and the reaction was stirred at RT until completion. The reaction mixture was diluted with EtOAc and the resulting mixture was washed with saturated NH₄Cl and brine, dried over Na₂SO₄, filtered and concentrated under vacuum. The crude product is then purified by flash chromatography or prep HPLC.

### Step 1—Method B

To a mixture of the appropriate carboxylic acid intermediate (1 equiv.) and Boc-guanidine (2 equiv.) in dry DMF (0.2M), N-Methylmorpholine (4 equiv.) and BOP (1.5 equiv.) were added. The resulting mixture was stirred at RT for several hours until completion. The reaction mixture was diluted with saturated NH₄Cl and extracted with EtOAc (×3). The combined organic layers were dried over Na₂SO₄, filtered and concentrated under vacuum. The crude product is then purified by flash chromatography or prep HPLC.

### Step 2—MOM and Boc Deprotection

To a solution of acylguanidine (1 equiv.) in DCM (0.1M) TFA (40 equiv.) was added at 0° C. and the reaction was slowly warmed to RT and stirred until completion. The solvent was removed under reduced pressure and the crude was purified by prep HPLC.

### Step 2—Benzyl Deprotection Followed by Boc Deprotection

To a solution of acylguanidine (1 equiv.) in Ethyl Acetate Pd 10% on carbon (0.1 equiv.) was added and reaction stirred under hydrogen until completion. The reaction mixture was then filtered through Celite and the solvent was removed under reduced pressure. The crude intermediate was then dissolved in DCM (0.1M) and TFA (20 equiv.) was added at 0° C. and the reaction was slowly warmed to RT and stirred until completion. The solvent was removed under reduced pressure and the crude was purified by prep HPLC.

### General Procedure B

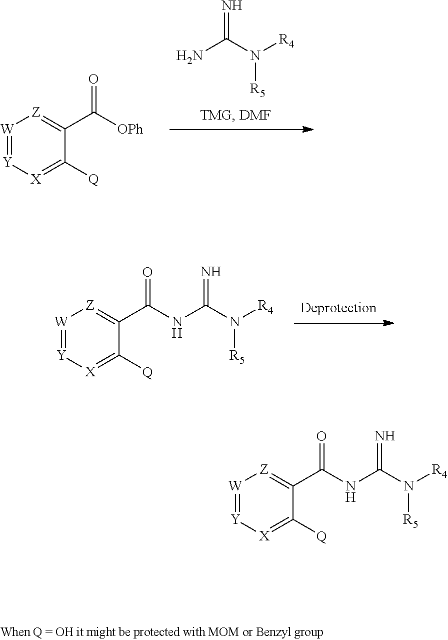

### Step 1

To a solution of the appropriate phenylester intermediate (1 equiv.) in DMF (0.2M) 1,1,3,3-Tetramethylguanidine (1.5 equiv.) was added followed by mono- or bis-substituted guanidine (2 equiv.) and the resulting reaction mixture was stirred at RT for several hours until completion. The reaction mixture was then diluted with water and extracted with EtOAc (×3). The combined organic layers were dried over Na₂SO₄, filtered and concentrated under vacuum. The crude product was then purified by prep HPLC.

Step 2—See deprotection steps in General Procedure A of acylguanidine synthesis.

### General Procedure C

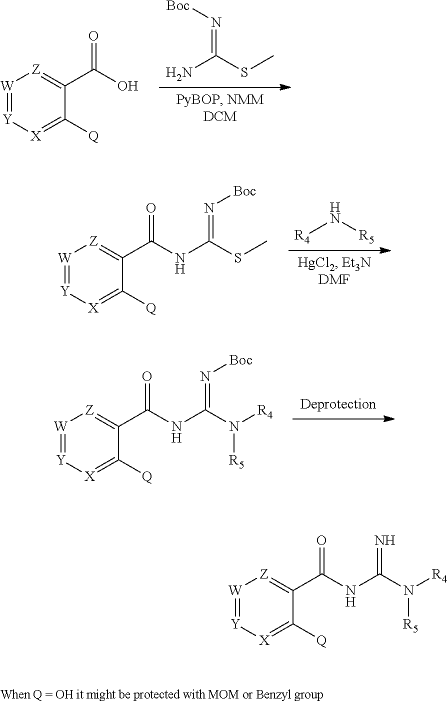

### Step 1

The carboxylic acid intermediate (1 equiv.) was dissolved in dry DMF (0.2M), then NMM (4 equiv.) and PyBOP (1.5 equiv.) were added followed by test-butyl N-[(methylsulfanyl) methanimidoyl]carbamate (1.1 eq). The reaction was stirred at RT until completion. The reaction mixture was then diluted with EtOAc and washed with saturated NH₄Cl and brine. The organic solution was then dried over Na₂SO₄, filtered and concentrated under vacuum. The crude product was then purified by flash chromatography.

### Step 2

The above intermediate (1 equiv.) was then dissolved in dry DMF (0.2M) and Et₃N (10 equiv.) was added followed by HgCl₂ (1 equiv.) and the appropriate mono- or bis-substituted amine (1 eq). The reaction was stirred at RT until completion. The reaction mixture was then diluted with EtOAc and filtered through Celite. The filtrate was washed with saturated NH₄Cl and brine. The organic solution was then dried over Na₂SO₄, filtered and concentrated under vacuum. The crude product was then purified by flash chromatography.

Step 3—See deprotection steps in General Procedure A of acylguanidine synthesis.

### General Procedure D

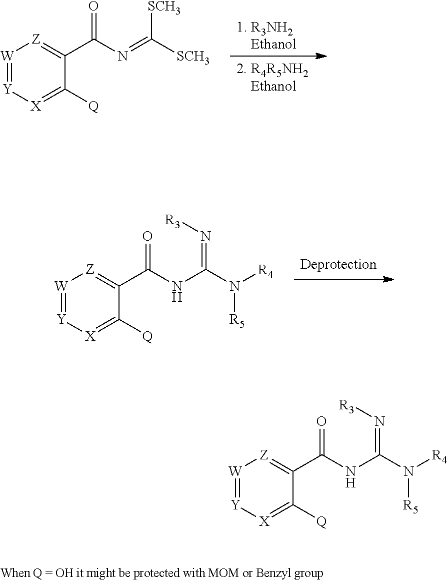

### Step 1

The appropriate S,S-dimethyl N-aroylcarbimidodithiolate intermediate (1 equiv.) was dissolved in Ethanol (0.2M) and the appropriate primary amine (1 equiv.) was added. The reaction was stirred at RT until completion. Then the solvent was evaporated under vacuum and the crude intermediate was then reacted in Ethanol (0.2M) with the appropriate mono- or bis-substituted amine (1.5 eq). The reaction was stirred at RT until completion. The reaction mixture was diluted with water and extracted with EtOAc (×3). The combined organic layers were dried over Na₂SO₄, filtered and concentrated under vacuum. The crude was then purified by prep HPLC.

Step 2—See deprotection steps in General Procedure A of acylguanidine synthesis.

### General Procedure E

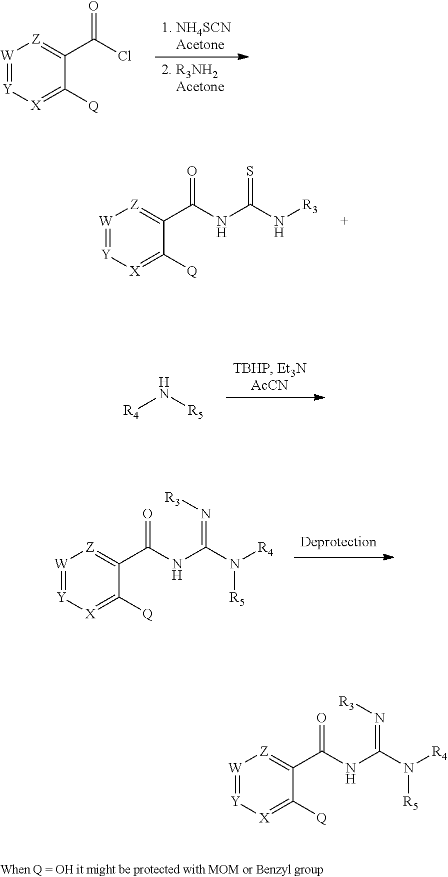

### Step 1

Suitable benzoyl chloride (1 eq, 11 mmol) was added to ammonium thiocyanate (1 equiv.) in acetone (0.4M). The reaction mixture was refluxed for 15 min and then cooled down to RT. An acetone solution of the appropriate primary amine (1 equiv.) was added and reaction refluxed for further 30 min or at RT for 3 hours. The reaction mixture was then poured into crushed ice and the resulting mixture was rigorously stirred. The solid was then filtered off and washed with water and used as crude for next step.

### Step 2

In a round bottomed flask charged with a solution of the crude benzoylthioureia (1 equiv.) in acetonitrile (0.1M) under vigorous magnetic stirring and cooled to 0° C. were added, respectively, trimethylamine (1 equiv.), the suitable nucleophilic mono- or bis-substituted amine (1 equiv.) and 70% aqueous tert-butyl hydroperoxide (3 eq). The reaction was slowly warmed to RT and after consumption of starting material the mixture was transferred to a separatory funnel charged with a NaHSO₃ saturated solution and extracted with DCM (×3). The combined organic layers were dried over Na₂SO₄, filtered and concentrated under reduced pressure. The crude was then purified by prep HPLC.

Step 3—See deprotection steps in General Procedure A of acylguanidine synthesis.

### General Procedure F

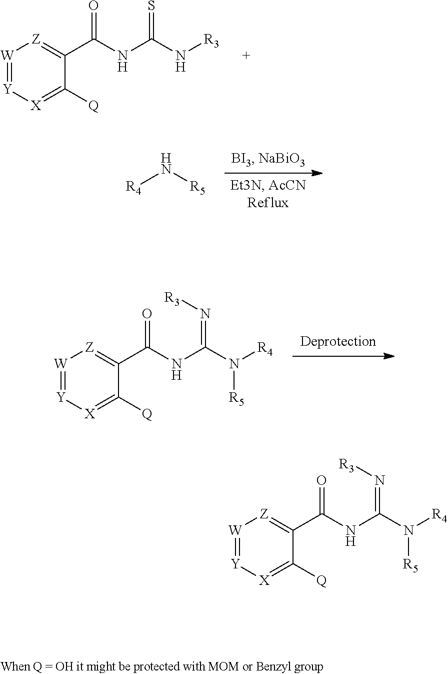

### Step 1

To a solution of the appropriate thiourea (1 eq, synthesized as in step 1 of general procedure E) in CH₃CN (0.1M) were added the appropriate mono- or bis-substituted amine (1 equiv.) and Et₃N (2 equiv.), followed by NaBiO₃ (1 equiv.) and BiI₃ (0.5 eq). The suspension was left stirring for 10 min at RT and became black. After this time, reaction was refluxed until completion. The solvent was then evaporated and DCM was added. The suspension was filtered through a pad of Celite and the pad washed twice with DCM. The filtrate was dried over anhydrous Na₂SO₄, filtered and the solvent was evaporated under vacuum. The crude product was purified by prep HPLC.

Step 2—See deprotection steps in General Procedure A of acylguanidine synthesis.

### General Procedure G

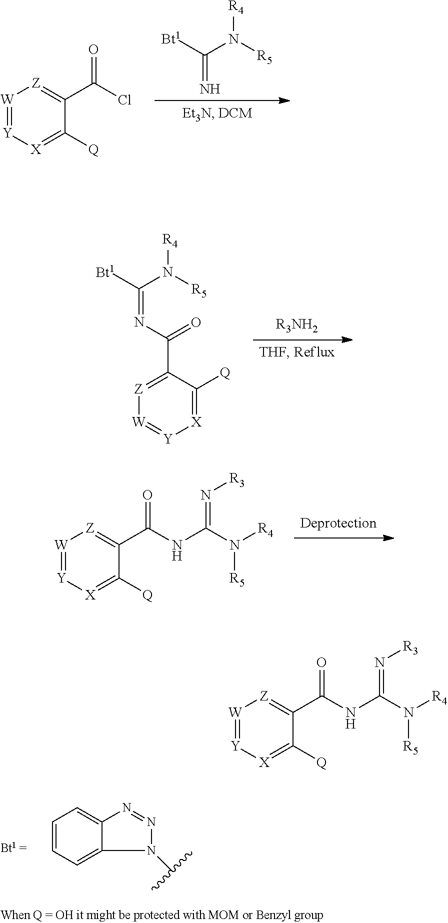

### Step 1

To a stirred solution of appropriate 1H-benzotriazol-1-ylcarboximidamide derivative (1 equiv.) in DCM (0.3M), the appropriate acid chloride (1 equiv.) was added at RT followed by the addition of triethylamine (1 eq). The reaction mixture was stirred at RT overnight. Upon completion, the reaction mixture was washed twice with water, dried over Na₂SO₄, filtered and concentrated under vacuum. The crude product was then purified by flash chromatography.

### Step 2

To a solution of N-acyl-1H-benzotriazol-1-ylcarboximidamide derivatives (1 equiv.) in THF (0.1M), the amine of choice (1 equiv.) was added. The reaction mixture was then refluxed until full conversion of starting materials. Upon completion, the solvent was evaporated under reduced pressure and the crude product was dissolved in DCM, washed twice with saturated aqueous sodium carbonate, dried over Na₂SO₄, and filtered. The solvent was removed under reduced pressure and the crude was purified by prep HPLC.

Step 3—See deprotection steps in General Procedure A of acylguanidine synthesis.

### Synthesis of Thiolylguanidines

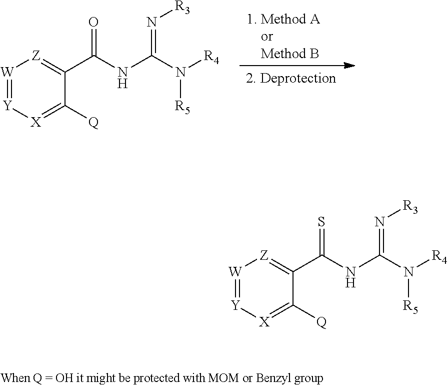

### Step 1—Method A

The appropriate acylguanidine (1 equiv.) was dissolved in Toluene (0.05M), then Lawesson's reagent was added (1 equiv.) and reaction refluxed until completion. Solvent was then removed under vacuum and crude was purified by prep HPLC.

### Step 1—Method B

The appropriate acylguanidine (1 equiv.) was dissolved in pyridine and phosphorus pentasulfide (1 equiv.) was added and reaction refluxed until completion. The reaction was then cooled down and poured into ice water. The resulting mixture was then extracted with EtOAc (×3) and the combined organic layers were dried over Na₂SO₄, filtered and concentrated under vacuum. The crude was then purified by prep HPLC

Step 2—See deprotection steps in General Procedure A of acylguanidine synthesis.

### Synthesis of Cyclic Acylguanidines

### General Procedure A

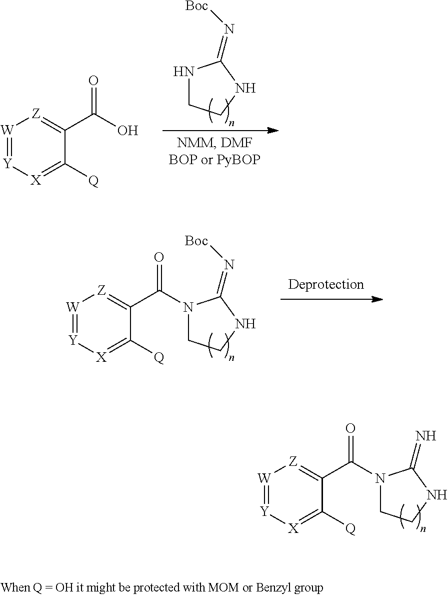

### Step 1

To a mixture of the appropriate carboxylic acid intermediate (1 equiv.) and the appropriate Boc-cyclicguanidine (2 equiv.) in dry DMF (0.2M) N-Methylmorpholine (4 equiv.) and BOP or PyBOP (1.5 equiv.) were added. The resulting mixture was stirred at RT for several hours until completion. The reaction mixture was diluted with saturated NH₄Cl and extracted with EtOAc (×3). The combined organic layers were dried over Na₂SO₄, filtered and concentrated under vacuum. The crude product was then purified by column chromatography or prep HPLC.

Step 2—See deprotection steps in General Procedure A of acylguanidine synthesis.

### General Procedure B

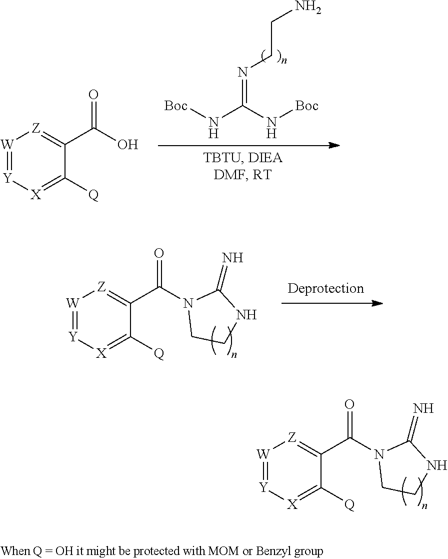

### Step 1

To a solution of the appropriate carboxylic acid intermediate (1 equiv.) and TBTU (1.5 equiv.) was added the appropriate di-Boc-guanidine (1.5 equiv.) and DIEA (2.5 eq). Reaction was stirred at RT until completion. The crude was purified by prep HPLC.

Step 2—See deprotection steps in General Procedure A of acylguanidine synthesis.

### General Procedure C

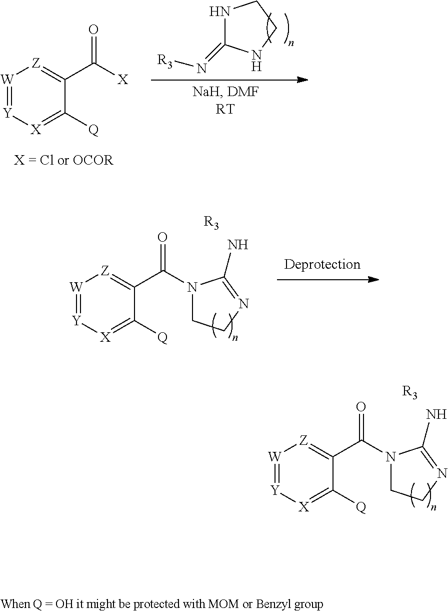

### Step 1

The appropriate cyclic guanidine (1 equiv.) was dissolved in DMF (0.1M) then NaH (2.5 equiv.) was added and reaction stirred at RT for 30 min before the appropriate acyl chloride or anhydride (2.5 equiv.) was added. Reaction stirred at RT until completion. The reaction mixture was diluted with water and extracted with DCM (×3). The combined organic layers were dried over Na₂SO₄, filtered and concentrated under vacuum. The crude product was purified by prep HPLC.

Step 2—See deprotection steps in General Procedure A of acylguanidine synthesis.

### General Procedure D

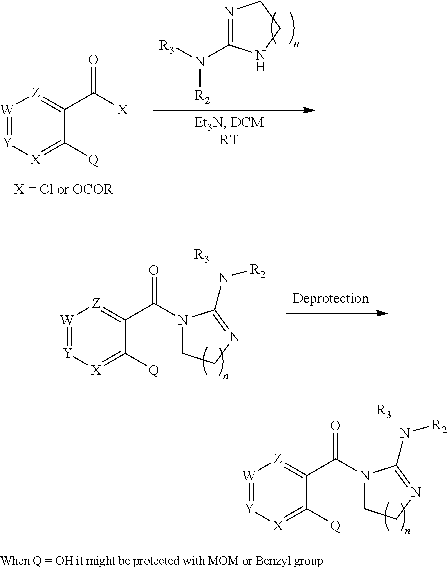

### Step 1

To a solution of the appropriate cyclic guanidine (1 equiv.) and triethylamine (1.5 equiv.) in DCM (0.1M) the appropriate acyl chloride or anhydride (1 equiv.) was added at 0° C. and reaction was slowly warmed to RT and stirred until completion. The reaction mixture was then washed with water, dried over Na₂SO₄, filtered and concentrated under vacuum. The crude product was purified by prep HPLC.

Step 2—See deprotection steps in General Procedure A of acylguanidine synthesis.

### General Procedure E

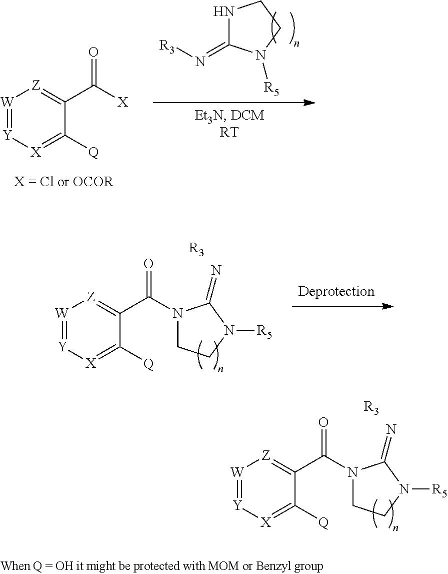

### Step 1

To a solution of the appropriate cyclic guanidine (1 equiv.) and triethylamine (1.5 equiv.) in DCM (0.1M) the appropriate acyl chloride or anhydride (1 equiv.) was added at 0° C. and reaction was slowly warmed to RT and stirred until completion. The reaction mixture was then washed with water, dried over dried over Na₂SO₄, filtered and concentrated under vacuum. The crude product was purified by prep HPLC.

Step 2—See deprotection steps in General Procedure A of acylguanidine synthesis.

### General Procedure F

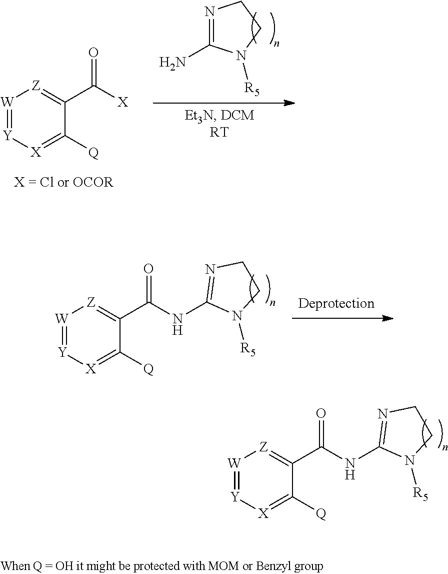

### Step 1

To a solution of the appropriate cyclic guanidine (1 equiv.) and triethylamine (1.5 equiv.) in DCM (0.1M) the appropriate acyl chloride or anhydride (1 equiv.) was added at 0° C. and reaction was slowly warmed to RT and stirred until completion. The reaction mixture was then washed with water, dried over dried over Na₂SO₄, filtered and concentrated under vacuum. The crude product was purified by prep HPLC.

Step 2—See deprotection steps in General Procedure A of acylguanidine synthesis.

### Synthesis of Cyclic Thiolylguanidines

The synthesis of cyclic thiolylguanidines is performed starting from cyclic acylguanidines, whose synthesis is described in the section above, using the reaction conditions described in the synthesis of thiolylguanidines section.

### Synthesis of Sulfaguanidines

### General Procedure A

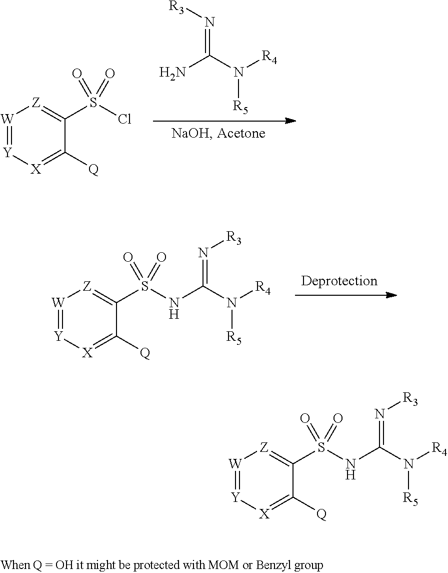

### Step 1

The appropriate guanidine (1 equiv.) was dissolved in aqueous NaOH and stirred at RT for 45-60 min, then Acetone was added (0.1M) and the reaction mixture was cooled to 0° C. and the appropriate sulfonyl chloride (1 equiv.) was added and reaction was slowly warmed to RT and stirred until completion. 1 HCl was added to bring the pH to acidic and then the mixture was extracted with DCM (×3). The combined organic layers were dried over Na₂SO₄, filtered and concentrated under vacuum. The crude product was purified by prep HPLC.

Step 2—See deprotection steps in General Procedure A of acylguanidine synthesis.

### General Procedure B

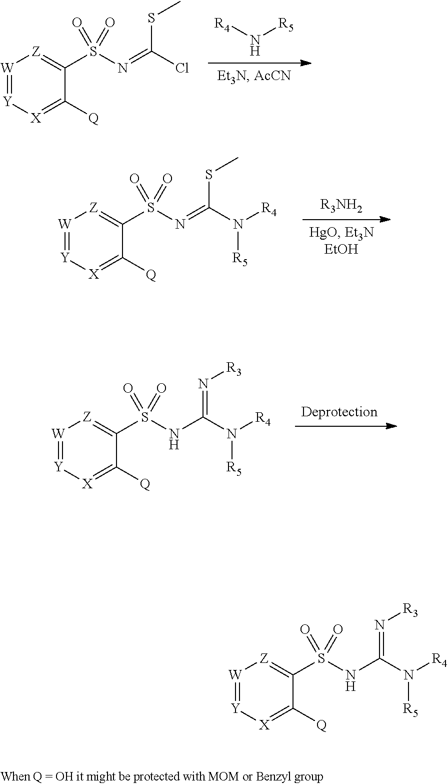

### Step 1

A flame dried flask equipped with a stirbar was cooled under a stream of nitrogen and charged with the appropriate carbonochloridoimidothioate (1 equiv.) and anhydrous acetonitrile (0.15 M). The mixture was cooled to 0° C. then triethylamine (1.2) was added dropwise. The appropriate amine (1.1 equiv.) was then added dropwise as a solution in acetonitrile (1.5 mL/mmol of amine). The reaction mixture was then warmed to RT and stirred until completion. The solvent was removed under reduced pressure, and the crude product was purified by flash chromatography or prep HPLC.

### Step 2

A flame-dried round bottom flask equipped with a stir bar was cooled under a stream of nitrogen and charged with the above intermediate (1 equiv.), mercuric oxide (1.5 eqs), and triethylamine (4.5 equiv.), followed by the appropriate amine (3 to 5 equiv.) and the mixture was stirred at RT until completion. The solution was then filtered through Celite, and the solvent was removed under reduced pressure. The crude product was purified by prep HPLC.

Step 3—See deprotection steps in General Procedure A of acylguanidine synthesis.

### General Procedure C

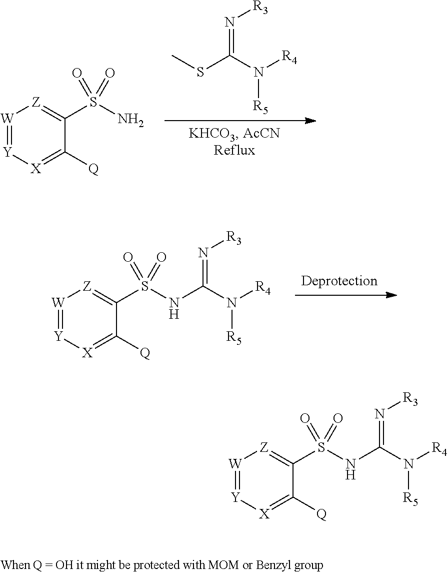

### Step 1

To a solution of the appropriate thiomethyl derivative (1 equiv.) in acetonitrile (0.2M), KHCO₃ (1.5 equiv.) and the appropriate sulfonamide (1.1 equiv.) was added and the reaction was refluxed until completion. The reaction mixture was diluted with water and the resulting mixture was extracted with DCM (×3). The combined organic layers were dried over Na₂SO₄, filtered and concentrated under vacuum. The crude product was purified by prep HPLC.

Step 2—See deprotection steps in General Procedure A of acylguanidine synthesis.

### Synthesis of Cyclic Sulfaguanidines

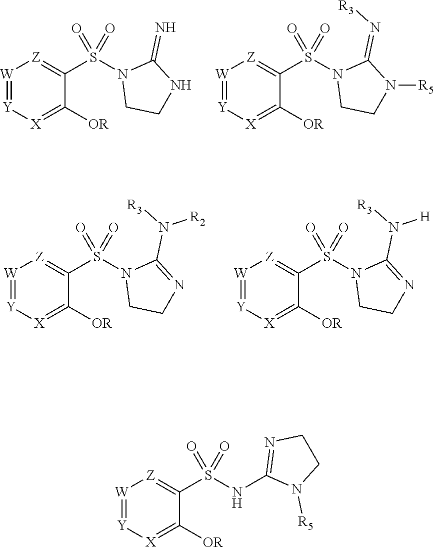

These final compounds are prepared using the same reaction conditions than the cyclic acyllguanidines starting from the appropriate sulfonyl chlorides and cyclic guanidines.

### Synthesis of Guanidines

### General Procedure A

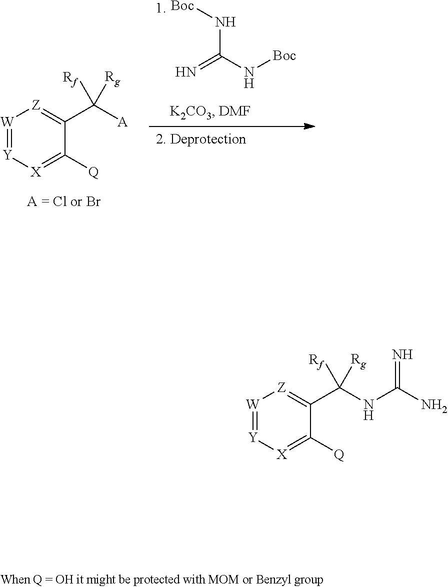

### Step 1

To a solution of Boc-protected guanidine (1.2 equiv.) in DMF (0.2M) potassium carbonate (1.5 equiv.) was added followed by the appropriate halide (1 equiv.) and reaction stirred at RT until completion. The reaction mixture was diluted with water and the resulting mixture was extracted with DCM (×3). The combined organic layers were dried Na₂SO₄, filtered and concentrated under vacuum. The crude product was purified by prep HPLC.

Step 2—See deprotection steps in General Procedure A of acylguanidine synthesis.

### General Procedure B

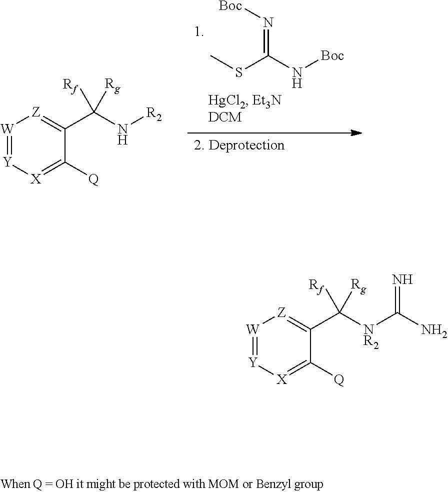

### Step 1

To an ice-cooled solution of the appropriate amine (1.5 equiv.) in DCM (0.15M) was added sequentially triethylamine (4.5 equiv.), 1,3-bis(ter/-butoxycarbonyl)-2-methyl-2-thiopseudourea (1 equiv.) and HgCl₂ (1 eq). The reaction was slowly warmed to RT and stirred until completion. The suspension was filtered through a plug of Celite and the filter-cake was washed with further DCM. The filtrate was washed sequentially with 10% aqueous citric acid, 10% aqueous potassium carbonate and brine. The organic phase was dried over Na₂SO₄, filtered and concentrated under vacuum. The crude product was purified by prep HPLC.

Step 2—See deprotection steps in General Procedure A of acylguanidine synthesis.

### General Procedure C

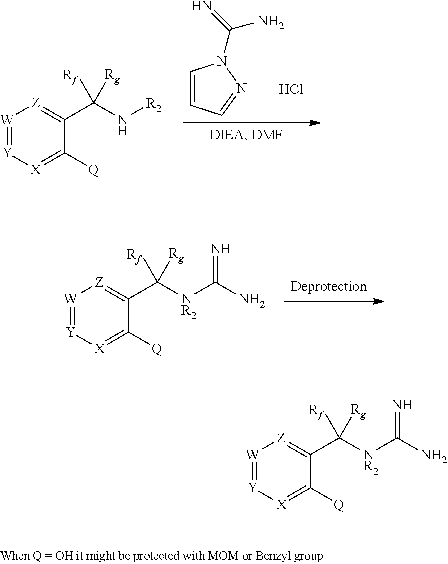

### Step 1

The appropriate amine (1 equiv.) was dissolved in a mixture of anhydrous DMF (0.3M) and DIPEA (8 eq). 1H-pyrazole-1-carboxamidine hydrochloride (2 equiv.) was then added, and the reaction mixture was stirred at RT until completion. Water was added and the resulting mixture was extracted with DCM (×3). The combined organic layers were dried over Na₂SO₄, filtered and concentrated under vacuum. The crude product was purified by prep HPLC.

Step 2—See deprotection steps in General Procedure A of acylguanidine synthesis.

### General Procedure D

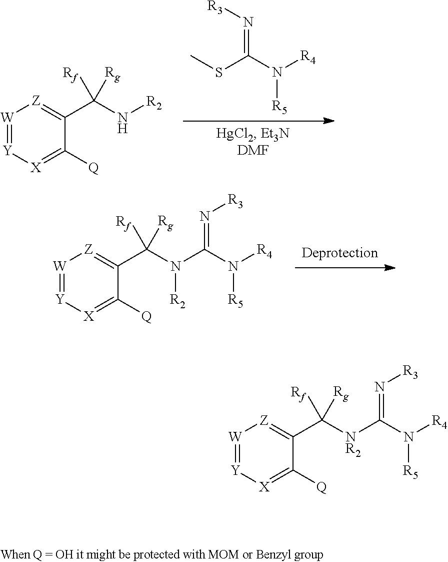

### Step 1

The appropriate amine (1 equiv.) was dissolved in dry DMF and Et₃N (10 equiv.) was added followed by HgCl₂ (1 equiv.) and the appropriate thiomethyl derivative (1 eq). The reaction was stirred at RT until completion. The reaction mixture was then diluted with EtOAc and filtered through Celite. The filtrate was washed with saturated NH₄Cl and brine. The organic solution was then dried over Na₂SO₄, filtered and concentrated under vacuum. The crude product was then purified by prep HPLC.

Step 2—See deprotection steps in General Procedure A of acylguanidine synthesis.

### General Procedure E

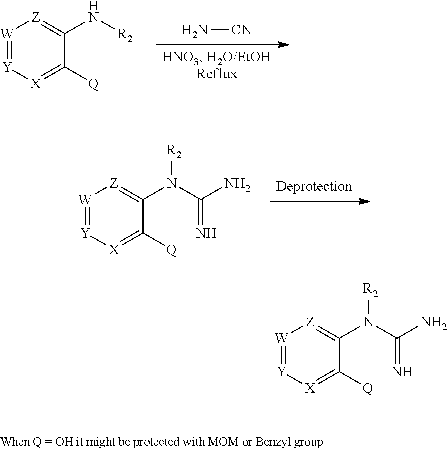

### Step 1

Nitric acid (69%) was added dropwise to a solution of the appropriate aniline derivative (1 equiv.) in EtOH (0.2M), followed by addition of a solution of cyanamide (5 equiv.) in a minimal amount of H₂O. The reaction mixture was heated at reflux for 18-36 hours, and then concentrated under vacuum. The crude product was purified by prep HPLC.

Step 2—See deprotection steps in General Procedure A of acylguanidine synthesis.

### General Procedure F

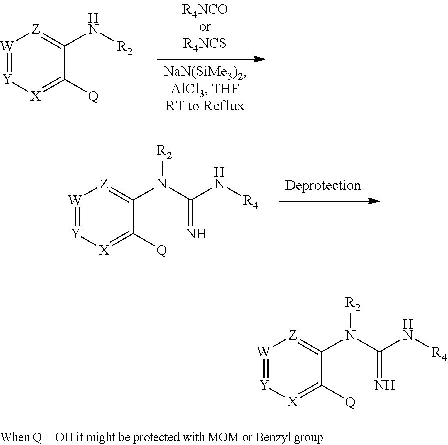

### Step 1

Isocyanates or isothiocyanates (1 equiv.) and sodium bis(trimethylsilyl)amide (2.0 M in THF, 1.2 equiv.) were added into a two-necked flask at RT and reaction stirred under nitrogen for 1 h. After isocyanates or isothiocyanates were completely consumed and converted to the cyanamide anion intermediates, various appropriate aniline (2.2 equiv.), AlCl₃, (0.1 eq, 10% w/w)) were added and reaxction heated at reflux for 6-12 h under N₂. After the reaction was completed, the reaction mixture was filtrated, washed with DCM and concentrated under reduced pressure. The residue was purified by prep HPLC.

Step 2—See deprotection steps in General Procedure A of acylguanidine synthesis.

### General Procedure G

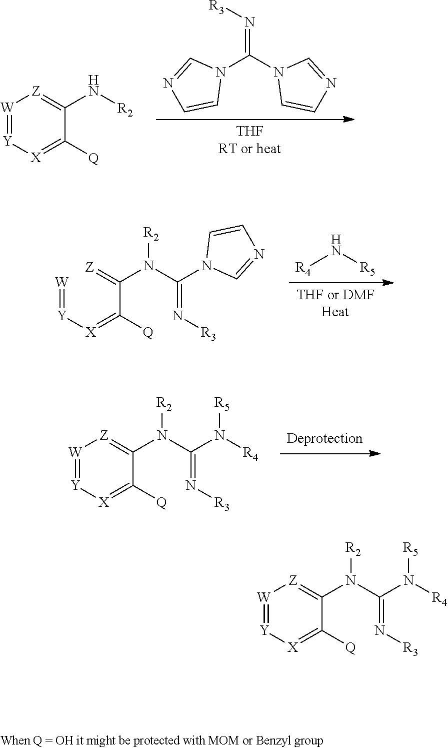

### Step 1

To a solution of the appropriate diimidazole derivative (1 equiv.) in THF (0.4M) the appropriate amine (1.2 equiv.) was added and reaction stirred at RT or 40° C. until completion. Water was then added and the resulting mixture was extracted with DCM (×3). The combined organic layers were dried over Na₂SO₄, filtered and concentrated under vacuum. The crude product was used for next step without any further purification.

### Step 2

The above intermediate (1 equiv.) was dissolved in THF or DMF (0.5M) and the appropriate amine (1.5 equiv.) was added and reaction heated until completion. Water was then added and the resulting mixture was extracted with DCM (×3). The combined organic layers were dried over Na₂SO₄, filtered and concentrated under vacuum. The crude product was purified by prep HPLC.

Step 3—See deprotection steps in General Procedure A of acylguanidine synthesis.

### Synthesis of Cyclic Guanidines

### General Procedure A

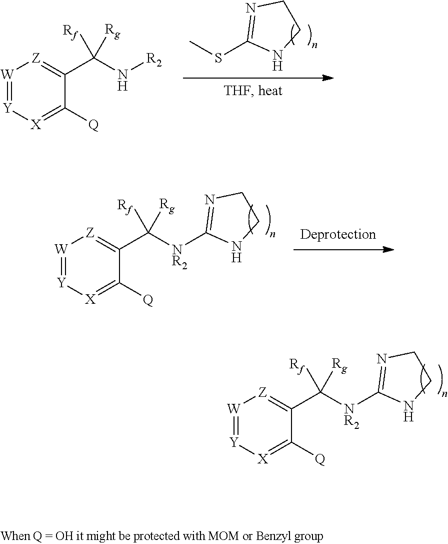

### Step 1

The appropriate amine (1.1 equiv.) was added to a solution of the appropriate cyclic methylisothiourea (1 equiv.) in dry THF (1M) and the reaction was stirred at 40° C. until completion. Water was added and the resulting mixture was extracted with DCM (×3). The combined organic layers were dried Na₂SO₄, filtered and concentrated under vacuum. The crude product was then purified by prep HPLC.

Step 2—See deprotection steps in General Procedure A of acylguanidine synthesis.

### General Procedure B

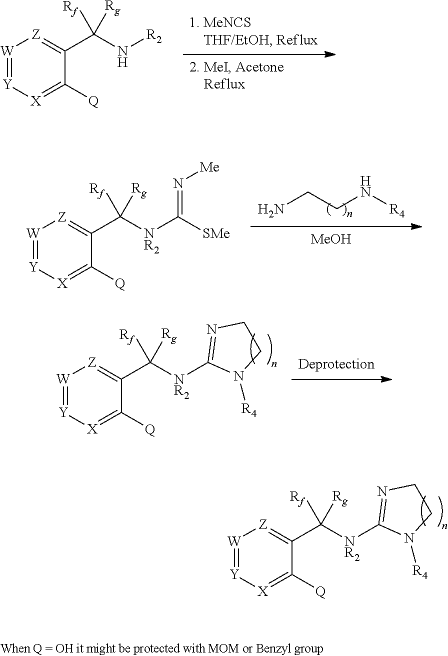

### Step 1

The appropriate amine (1 equiv.) was dissolved in a mixture of THF/EtOH (0.2M) then MeNCS (5 equiv.) was added and reaction refluxed until completion. Water was added and the resulting mixture was extracted with DCM (×3). The combined organic layers were dried Na₂SO₄, filtered and concentrated under vacuum. The crude product was used for next step without any further purification.

### Step 2

The above intermediate (1 equiv.) was dissolved in Acetone (0.1M) and MeI (5 equiv.) was added and reaction refluxed until completion. Water was added and the resulting mixture was extracted with DCM (×3). The combined organic layers were dried over Na₂SO₄, filtered and concentrated under vacuum. The crude product was then purified by flash chromatography.

### Step 3

The above intermediate (1 equiv.) was dissolved in MeOH (0.1M) and the appropriate diamine (2 equiv.) was added and reaction stirred at RT until completion. Solvent evaporated and crude was purified by prep HPLC.

Step 4—See deprotection steps in General Procedure A of acylguanidine synthesis.

### General Procedure C

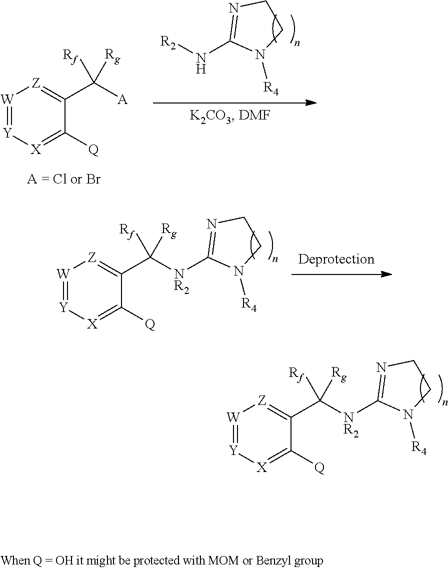

### Step 1

To a solution of the appropriate cyclic guanidine (1 equiv.) in DMF (0.2M) potassium carbonate (1.5 equiv.) was added followed by the appropriate halide (1.1 eq). The reaction was stirred at RT until completion. Water was added and the resulting mixture was extracted with DCM (×3). The combined organic layers were dried over Na₂SO₄, filtered and concentrated under vacuum. The crude product was then purified by prep HPLC.

Step 2—See deprotection steps in General Procedure A of acylguanidine synthesis.

### General Procedure D

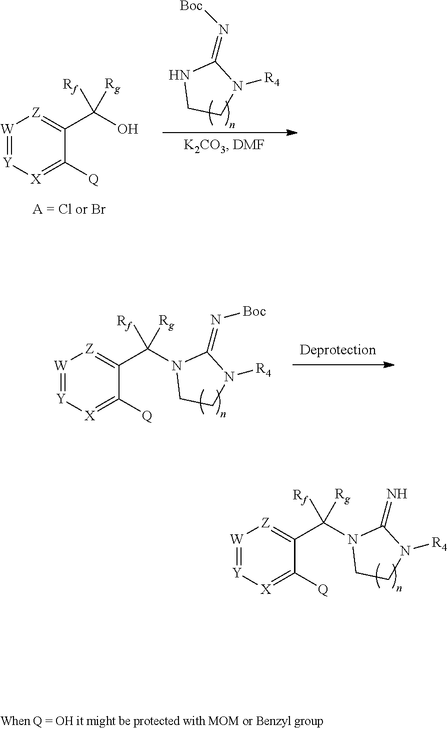

### Step 1

To a solution of the appropriate cyclic guanidine (1 equiv.) in DMF (0.2M) potassium carbonate (1.5 equiv.) was added followed by the appropriate halide (1.1 eq). The reaction was stirred at RT until completion. Water was added and the resulting mixture was extracted with DCM (×3). The combined organic layers were dried over Na₂SO₄, filtered and concentrated under vacuum. The crude product was then purified by prep HPLC.

Step 2—See deprotection steps in General Procedure A of acylguanidine synthesis.

### General Procedure E

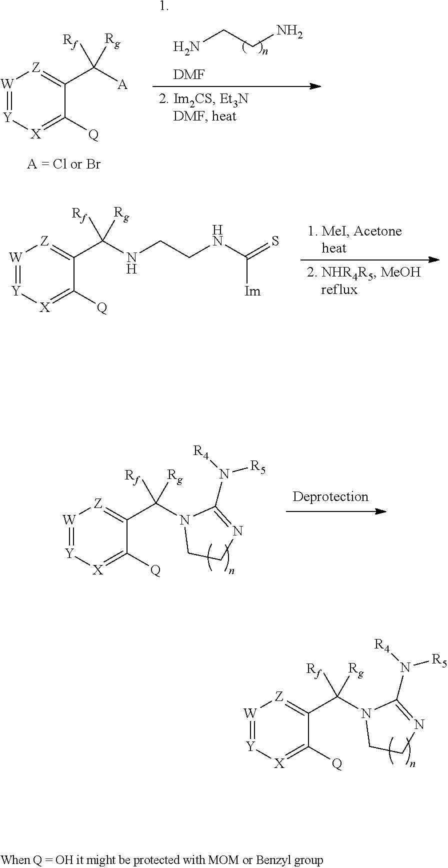

### Step 1

To a solution of the appropriate halide (1 equiv.) in DMF (0.2M) the appropriate diamine (5 equiv.) was added and reaction stirred at RT until completion. Water was added and the resulting mixture was extracted with DCM (×3). The combined organic layers were dried over Na₂SO₄, filtered and concentrated under vacuum. The crude product was used for next step without any further purification.

### Step 2

The above intermediate (1 equiv.) was dissolved in DMF (0.2M) then triethylamine (5 equiv.) was added followed by Im₂CS (1 eq). Reaction was stirred at 70° C. until completion. Water was added and the resulting mixture was extracted with DCM (×3). The combined organic layers were dried over Na₂SO₄, filtered and concentrated under vacuum. The crude product was then purified by flash chromatography.

### Step 3

The above intermediate (1 equiv.) was dissolved in Acetone (0.1M) and MeI (5 equiv.) was added and reaction refluxed until completion. Water was added and the resulting mixture was extracted with DCM (×3). The combined organic layers were dried over Na₂SO₄, filtered and concentrated under vacuum. The crude product was used for next step without any further purification.

### Step 4

The above intermediate (1 equiv.) was dissolved in MeOH (0.1M) and the appropriate amine (5 equiv.) was added and reaction refluxed until completion. Solvent was evaporated and the crude product was purified by prep HPLC.

Step 5—See deprotection steps in General Procedure A of acylguanidine synthesis.

### General Procedure F

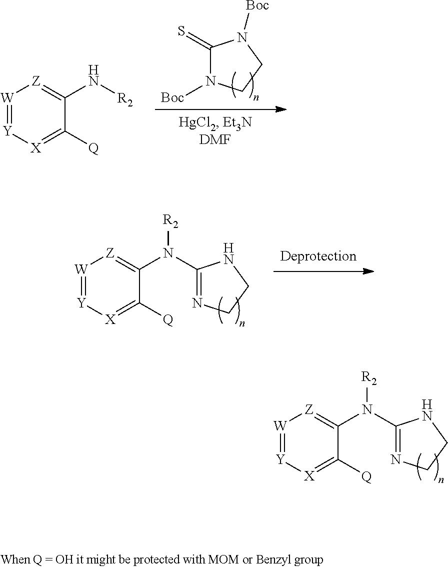

### Step 1

To a solution of the appropriate amine (1 equiv.) in DMF (0.2M) the appropriate cyclic thiourea (1.2 equiv.), triethylamine (5 equiv.) and HgCl₂ (1 equiv.) were added and the reaction was stirred at RT until completion. The reaction mixture was then diluted with EtOAc and filtered through Celite. The filtrate was washed with saturated NH₄Cl and brine. The organic solution was then dried over Na₂SO₄, filtered and concentrated under vacuum. The crude product was then purified by prep HPLC.

Step 2—See deprotection steps in General Procedure A of acylguanidine synthesis.

### General Procedure G

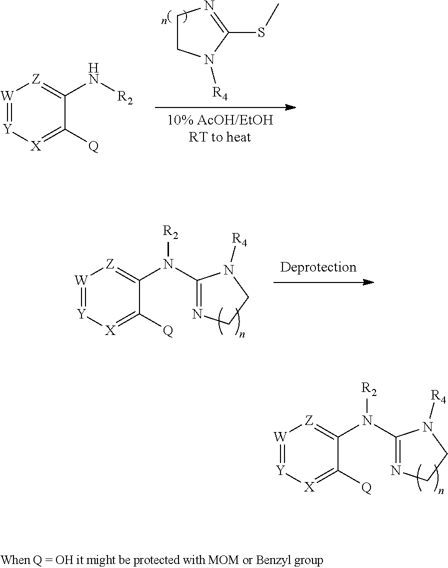

### Step 1

The appropriate amine (1 equiv.) was dissolved in 10% AcOH/EtOH (0.2M) and the appropriate cyclic thiomethyl reagent (1.5 equiv.) was added at RT. Reaction then was heated and stirred until completion. Solvent was removed under vacuum and the crude product was purified by prep HPLC.

Step 2—See deprotection steps in General Procedure A of acylguanidine synthesis.

### General Procedure H

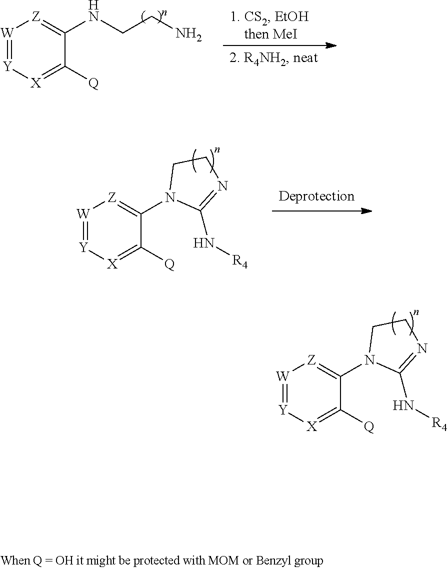

### Step 1

A solution of the appropriate diamine derivative (1 equiv.) in ethanol (0.5M) was stirred while carbon disulfide (1 equiv.) was added dropwise, and the mixture was heated at 50° C. for 3 h. The solution was then cooled, and the precipitated intermediate was collected. MeI (2.5 equiv.) was added dropwise to a suspension of the above solid (1 equiv.) in methanol (0.5M) and the reaction mixture heated at 60° C. until starting material disappeared. The reaction mixture was concentrated under vacuum to an oil, and then excess of the appropriate amine (10 equiv.) was added. The mixture was then heated at 90° C. until completion. 0.1N aqueous NaOH was added and the resulting solution was extracted with EtOAc (×3). The combined organic layers were washed with brine, dried over Na₂SO₄, filtered and concentrated under vacuum. The crude mixture was purified by prep HPLC.

Step 2—See deprotection steps in General Procedure A of acylguanidine synthesis.

### General Procedure I

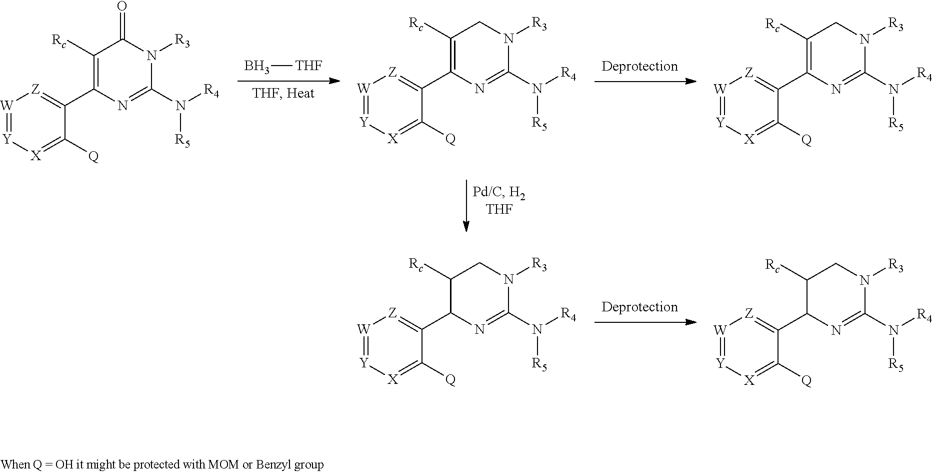

### Step 1

To a solution of the appropriate starting material (1 equiv.) in THF (0.2M) BH₃-THF (2M in THF, 3 equiv.) was added and reaction refluxed until full conversion. Reaction was cooled down to RT and 1M HCl was added and the reaction mixture was stirred at RT until the two layers are well separated. The mixture was then extracted with EtOAc (×3) and the combined organic layers were dried over Na₂SO₄, filtered and concentrated under vacuum. The crude was purified by prep HPLC.

### Step 2

The above intermediate (1 equiv.) was dissolved in THF (0.1M) and 10% Palladium on carbon (0.1 eqs) was added and reaction stirred under hydrogen atmosphere until completion. The reaction mixture was filtered through a pad of Celite and solvent was removed under vacuum. The crude product was then purified by prep HPLC.

Step 3—See deprotection steps in General Procedure A of acylguanidine synthesis.

### General Procedure K

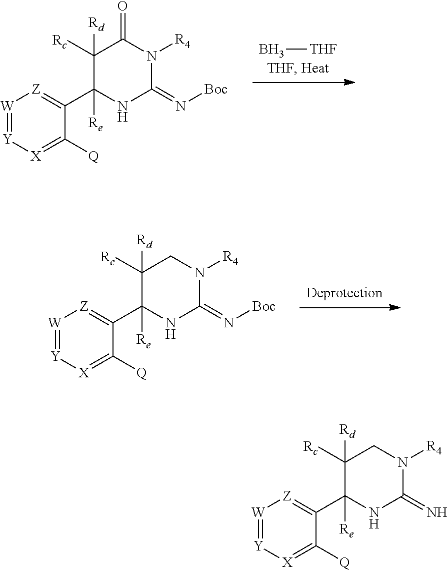

### Step 1

To a solution of the appropriate starting material (1 equiv.) in THF (0.2M) BH₃-THF (2M in THF, 3 equiv.) was added and reaction refluxed until full conversion. Reaction was cooled down to RT and 1M HCl was added and the reaction mixture was stirred at RT until the two layers are well separated. The mixture was then extracted with EtOAc (×3) and the combined organic layers were dried over Na₂SO₄, filtered and concentrated under vacuum. The crude was purified by prep HPLC.

Step 2—See deprotection steps in General Procedure A of acylguanidine synthesis.

### Synthesis of Aminoimidazoles and Aminoimidazolines

### General Procedure A

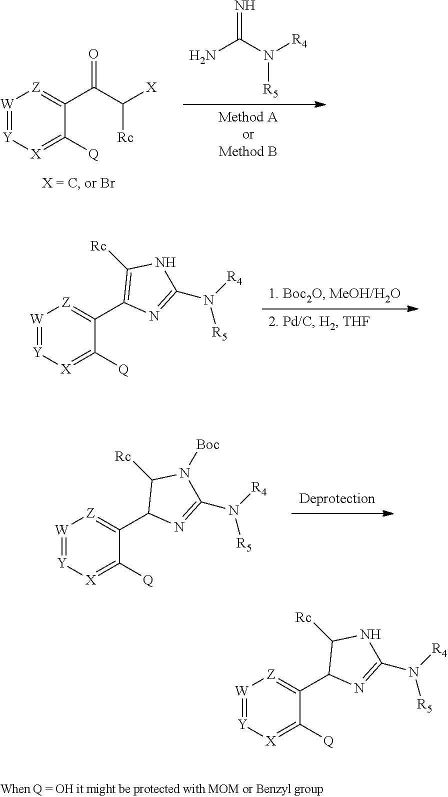

### Step 1—Method A

To a solution of the appropriate halide derivative (1 equiv.) in DMF (0.2M) was added the appropriate guanidine (2 equiv.) and reaction was stirred at RT until completion. The solvent was removed under vacuum and the imidazole product was purified by prep HPLC.

### Step 1—Method B

A mixture of the corresponding halide derivative (1 equiv.) and guanidine (3 equiv.) in anhydrous acetonitrile (0.1M) was heated at 100° C. using microwave irradiation for 15 min. The solvent was removed and the crude product was purified by prep HPLC.

### Step 2

To a suspension of the appropriate imidazole derivative (1 equiv.) in a mixture of methanol/water 2:1 (0.15M) Boc-anhydride (1.1 equiv.) was added and the mixture was stirred at RT until completion. The precipitate was filtered off, washed with methanol and the crude product was used for next reaction without any further purification.

### Step 3

The above intermediate (1 equiv.) was dissolved in THF (0.1M) and 10% Palladium on carbon (0.1 equiv.) was added and reaction stirred under hydrogen atmosphere until completion. The reaction mixture was filtered through a pad of Celite and solvent was removed under vacuum. The crude product was then purified by prep HPLC.

Step 4—See deprotection steps in General Procedure A of acylguanidine synthesis.

### General Procedure B

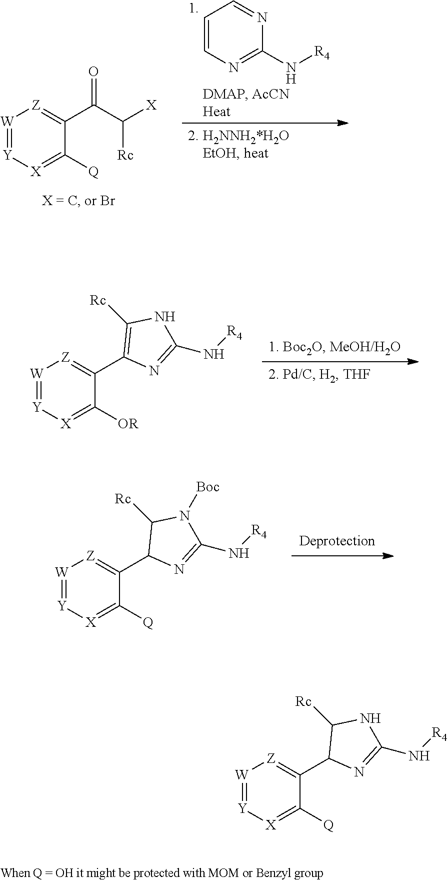

### Step 1

To a solution of the appropriate pyrimidine (1 equiv.) and halide derivative (1.2 equiv.) in acetonitrile (0.2M) DMAP (0.01 equiv.) was added. After being stirred at 85° C. until completion, the reaction mixture was filtered, washed with acetonitrile and ether and dried to give the pyrimidinium salt intermediate.

### Step 2

To a suspension of the above intermediate (1 equiv.) in ethanol (0.2M) hydrazine hydrate (35% hydrazine in solution, 7 equiv.) was added, and the reaction was placed in a microwave reactor and heated at 120° C. for 40 min. The mixture was cooled to RT, the solvent evaporated and the resulting residue was purified by prep HPLC.

### Step 3

To a suspension of the appropriate imidazole derivative (1 equiv.) in a mixture of methanol/water 2:1 (0.15M) Boc-anhydride (1.1 eqs) was added and the mixture was stirred at RT until completion. The precipitate was filtered off, washed with methanol and the crude product was used for next reaction without any further purification.

### Step 4

The above intermediate (1 equiv.) was dissolved in THF (0.1M) and 10% Palladium on carbon (0.1 eqs) was added and reaction stirred under hydrogen atmosphere until completion. The reaction mixture was filtered through a pad of Celite and solvent was removed under vacuum. The crude product was then purified by prep HPLC.

Step 5—See deprotection steps in General Procedure A of acylguanidine synthesis.

### General Procedure C

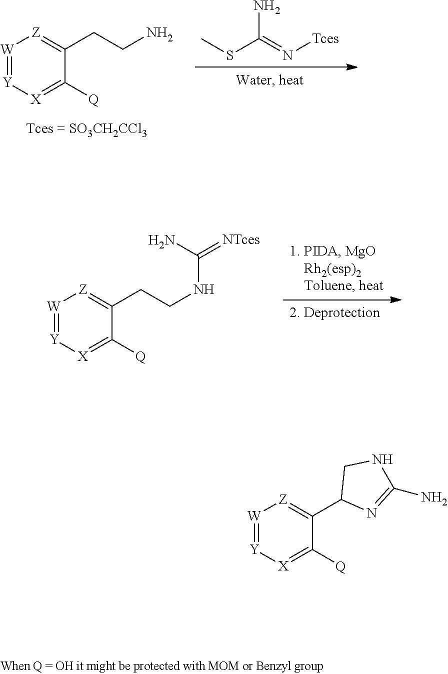

### Step 1

A flask was charged with S-methyl-N-(2,2,2 trichloroethoxysulfonyl)isothiourea (1 equiv.), the appropriate amine (1 equiv.), and H₂O (1M). The reaction was stirred at 100° C. until completion. The reaction mixture was cooled to RT and DCM was added. The biphasic solution was extracted with DCM (×3) and the combined organic layers were dried over Na₂SO₄, filtered and concentrated under reduced pressure. Purification of the isolated material by column chromatography afforded the desired guanidine substrate.

### Step 2

A flame-dried flask was charged with Tces guanidine (1 equiv.), Rh₂(esp)₂ (0.02 equiv.), PhI(OAc)₂ (1.65 equiv.), and MgO (2.5 eq). The reaction mixture was placed briefly under vacuum, and the flask then backfilled with nitrogen. This process was repeated two additional times prior to the addition of deoxygenated toluene (0.1M). The resulting suspension was heated to 40° C. and stirred until completion. The reaction mixture was then cooled to RT, and the crude was purified by prep HPLC.

Step 3—See deprotection steps in General Procedure A of acylguanidine synthesis.

### General Procedure D

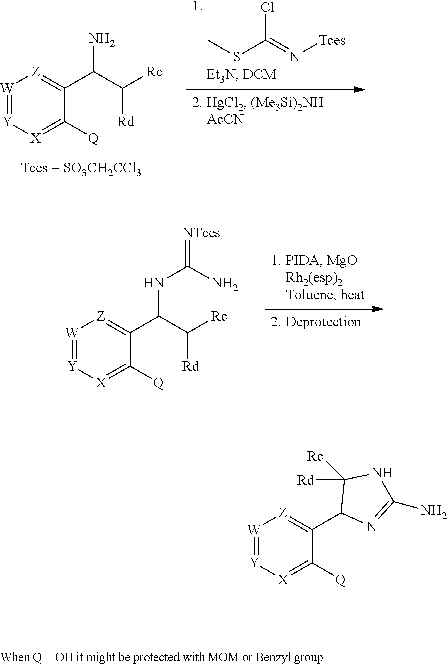

### Step 1

A solution of (2,2,2-trichloroethoxysulfonyl)carbonchloroimidothioicacid methyl ester (1.1 equiv.) in DCM (0.2M) was cooled to 0° C. and the appropriate amine (1 equiv.) was added dropwise. To the resulting mixture was added triethylamine (1.2 eq). The reaction was warmed to RT and stirred for 4 h. The solution was then concentrated under vacuum. Purification by chromatography on silica gel furnished the isothiourea product.

### Step 2

To a solution of isothiourea (1 equiv.) in MeCN (0.1M) was added successively (Me₃Si)₂NH (2.5 equiv.) and HgCl₂ (1.1 eq). After 1 h the milky white suspension was filtered through Celite and the filtrate was concentrated under reduced pressure. Purification of the crude residue by column chromatography gave the desired N-Tces guanidine.

### Step 3

A flame-dried flask was charged with Tces guanidine (1 equiv.), Rh₂ (esp)₂ (0.02 equiv.), PhI(OAc)₂ (1.65 equiv.), and MgO (2.5 eq). The reaction mixture was placed briefly under vacuum, and the flask then backfilled with nitrogen. This process was repeated two additional times prior to the addition of deoxygenated toluene (0.1M). The resulting suspension was heated to 40° C. and stirred until completion. The reaction mixture was then cooled to RT, and the crude was purified by prep HPLC.

Step 4—See deprotection steps in General Procedure A of acylguanidine synthesis.

### Synthesis of Aminopyrimidinone

### General Procedure A

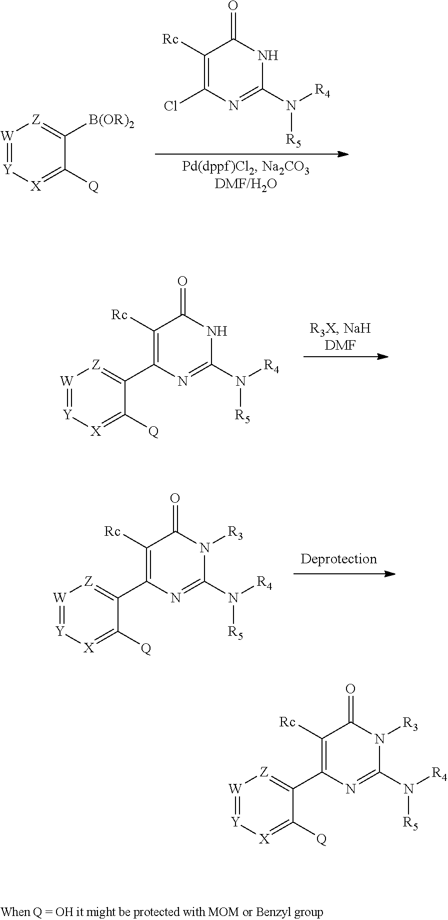

### Step 1

To a solution of the appropriate boronic acid (1 equiv.) in a mixture of DMF/Water 3:1 (0.1M), sodium carbonate (1.5 equiv.) was added followed by Pd(dppf)Cl₂ (0.1 equiv.) and reaction stirred at RT until completion. Water was added and the resulting mixture was extracted with DCM (×3). The combined organic layers were dried over Na₂SO₄, filtered and concentrated under vacuum. The crude product was then purified by prep HPLC.

Step 2 The above intermediate (1 equiv.) was dissolved in DMF (0.1 equiv.), then at 0° C. NaH (1.2 equiv.) was added and reaction stirred at RT for 30 min before the corresponding electrophile (1.5 equiv.) was added. Reaction was stirred at RT until completion. Water was added and the resulting mixture was extracted with DCM (×3). The combined organic layers were dried over Na₂SO₄, filtered and concentrated under vacuum. The crude product was then purified by prep HPLC.
Step 3—See deprotection steps in General Procedure A of acylguanidine synthesis.

### General Procedure B

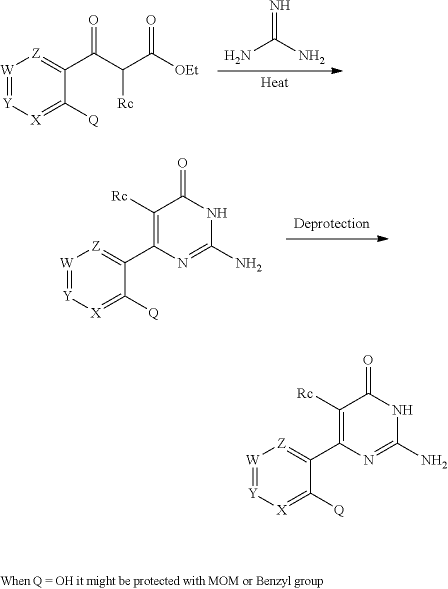

### Step 1

To a suspension of guanidine carbonate (1.5-5 equiv.) in ethanol (2 mL/mmol) was added the appropriate β-ketoester (1 equiv.), and the reaction mixture heated at 80° C. for 15-64 h. Following reaction completion by TLC, the mixture was cooled to RT, filtered and the crude product was purified by prep HPLC.

Step 2—See deprotection steps in General Procedure A of acylguanidine synthesis.

### Synthesis of Aminopyrimidinethione

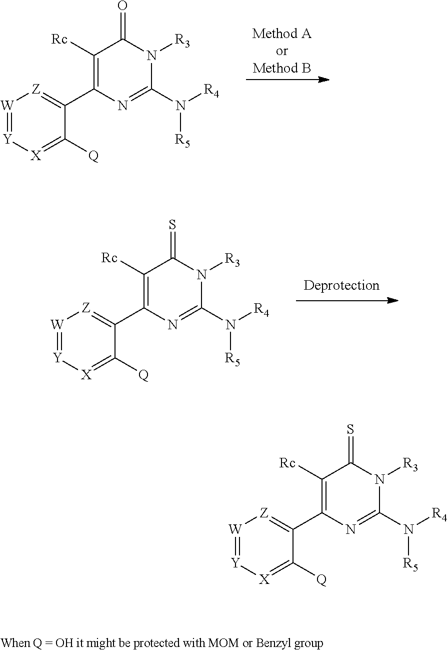

### Step 1—Method A

The appropriate starting material (1 equiv.) was dissolved in Toluene (0.05M), then Lawesson's reagent was added (1 equiv.) and reaction refluxed until completion. Solvent was then removed under vacuum and crude was purified by prep HPLC.

### Step 1—Method B

The appropriate starting material (1 equiv.) was dissolved in pyridine and phosphorus pentasulfide (1 equiv.) was added and reaction refluxed until completion. The reaction was then cooled down and poured into ice water. The resulting mixture was then extracted with Ethyl Acetate and the combined organic layers were dried over Na₂SO₄, filtered and concentrated under vacuum. The crude was then purified by prep HPLC

Step 2—See deprotection steps in General Procedure A of acylguanidine synthesis.

### Synthesis of Aminothiadiazine Dioxide

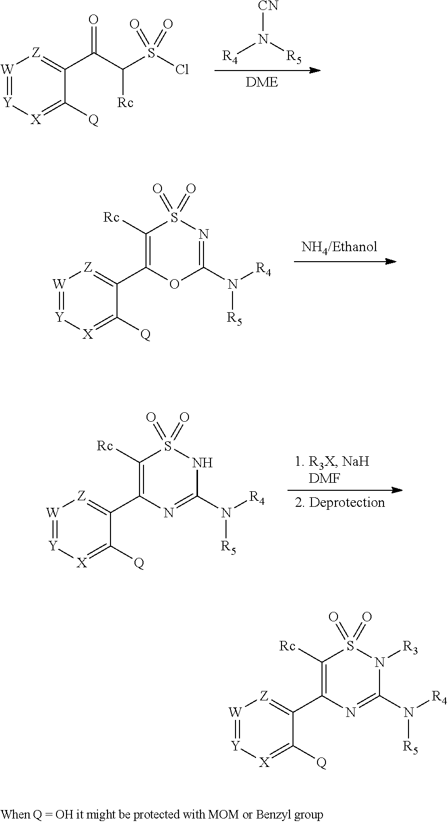

### Step 1

The appropriate sulfonyl chloride (1 equiv.) was dissolved in DME (0.1M) then the appropriate cyanamide (1.2 equiv.) was added and reaction stirred at 80° C. until completion. Solvent evaporated under reduced pressure and the crude product was purified by prep HPLC or flash chromatography.

### Step 2

The above intermediate (1 equiv.) was then dissolved in NH₄ in Ethanol and reaction stirred at RT until completion. Solvent evaporated under reduced pressure and the crude product was purified by prep HPLC.

### Step 3

The above product (1 equiv.) was dissolved in DMF (0.1 equiv.), then at 0° C. NaH (1.2 equiv.) was added and reaction stirred at RT for 30 min before the corresponding electrophile (1.5 equiv.) was added. Reaction was stirred at RT until completion. Water was added and the resulting mixture was extracted with DCM (×3). The combined organic layers were dried over Na₂SO₄, filtered and concentrated under vacuum. The crude product was then purified by prep HPLC.

Step 4—See deprotection steps in General Procedure A of acylguanidine synthesis.

### Synthesis of Iminotetrahydropyrimidinone

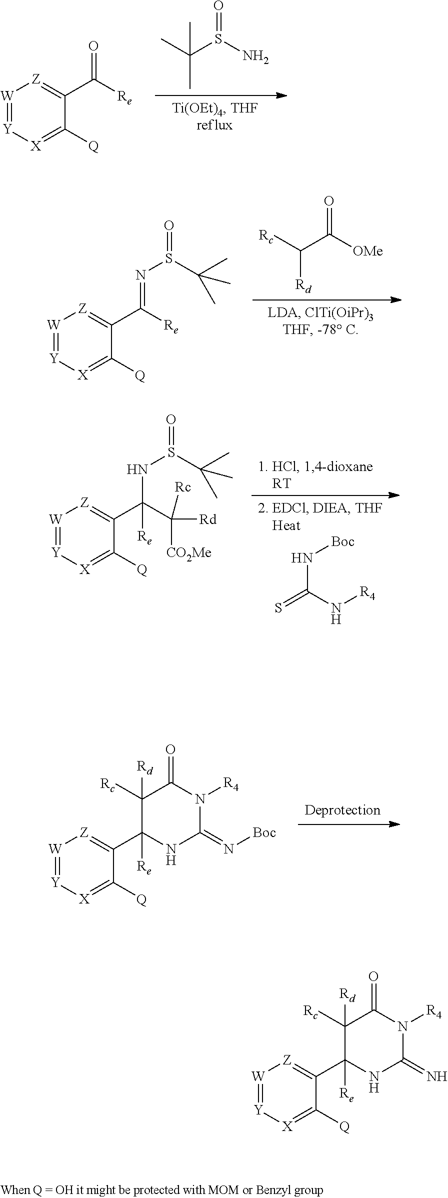

### Step 1

To a pressure vessel containing a solution of the appropriate ketone (1 equiv.) in THF (0.8M) was added the appropriate enantiomer of 2-methylpropane-2-sulfinamide (2.3 equiv.) followed by titanium(IV)ethoxide (0.45 eq). The vessel was sealed and the mixture was heated to 75° C. for several hours until completion. After that time, the mixture was cooled to room temperature, poured into water and then the mixture was filtered. The filter cake was washed with DCM and the filtrate was extracted with DCM. The organic layer was dried over Na₂SO₄, filtered, and concentrated under vacuum. The crude residue was purified by flash chromatography.

### Step 2

A solution of n-butyllithium (2.5 M in hexanes, 2.5 equiv.) was added slowly at 0° C. to a solution of DIEA (2.5 equiv.) in THF (0.9M) and the resulting solution was stirred at 0° C. for 0.5 h. The reaction mixture was then cooled to −78° C. followed by dropwise addition of a solution of the appropriate methyl acetate (2 equiv.) in THF (3.5M). The resulting reaction mixture was stirred at −78° C. for 1.5 h. After that time, a solution of chlorotitanium triisopropoxide (3 equiv.) in THF (2M) was added slowly and the reaction was stirred for 2 hours. A solution of the above intermediate (1 equiv.) in THF (12M) was then added slowly and the reaction stirred until completion. The reaction was quenched at −78° C. by gradual addition of water and the resulting mixture allowed to warm to RT overnight. The resultant slurry was diluted with EtOAc, filtered, and the filter cake was rinsed with EtOAc. The filtrate layers were separated and the organic layer was washed with water, dried over Na₂SO₄, filtered and concentrated under vacuum. The residue was purified by flash chromatography.

### Step 3

A solution of HCl (4.0 M in 1,4-dioxane, 8 equiv.) was added to a solution of the above intermediate (1 equiv.) in 7:1 methylene chloride/methanol (0.3M) and the reaction mixture stirred at RT until completion. The reaction mixture was then concentrated under vacuum and the residue dried under high vacuum to afford the crude intermediate amine-HCl salt.

### Step 4

To a portion of the amine (1 equiv.) in DMF (1M) was added N-(dimethylamino)propyl)-3-ethylcarbodiimide hydrochloride (1.5 equiv.) and DIEA (4.5 eq). The reaction mixture stirred at 45° C. until completion. After that time, the reaction mixture was diluted with water and EtOAc and the resultant mixture was stirred vigorously until the phases cleared. The layers were separated and the aqueous layer extracted with EtOAc (×2). The combined organic layers were sequentially washed with 1 N HCl, sat. Na₂CO₃, and brine, then dried over Na₂SO₄, filtered and concentrated under vacuum. The residue was purified by flash chromatography or prep HPLC.

Step 5—See deprotection steps in General Procedure A of acylguanidine synthesis.

### Synthesis of Iminotetrahydropyrimidinethione

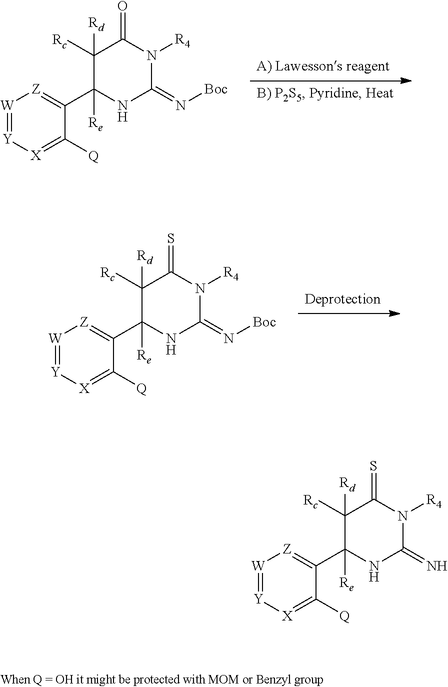

### Step 1—Method A

The appropriate starting material (1 equiv.) was dissolved in Toluene (0.05M), then Lawesson's reagent was added (1 equiv.) and reaction refluxed until completion. Solvent was then removed under vacuum and crude was purified by prep HPLC.

### Step 1—Method B

The appropriate starting material (1 equiv.) was dissolved in pyridine and phosphorus pentasulfide (1 equiv.) was added and reaction refluxed until completion. The reaction was then cooled down and poured into ice water. The resulting mixture was then extracted with Ethyl Acetate and the combined organic layers were dried over Na₂SO₄, filtered and concentrated under vacuum. The crude was then purified by prep HPLC

### Synthesis of Iminothiadiazinane Dioxide

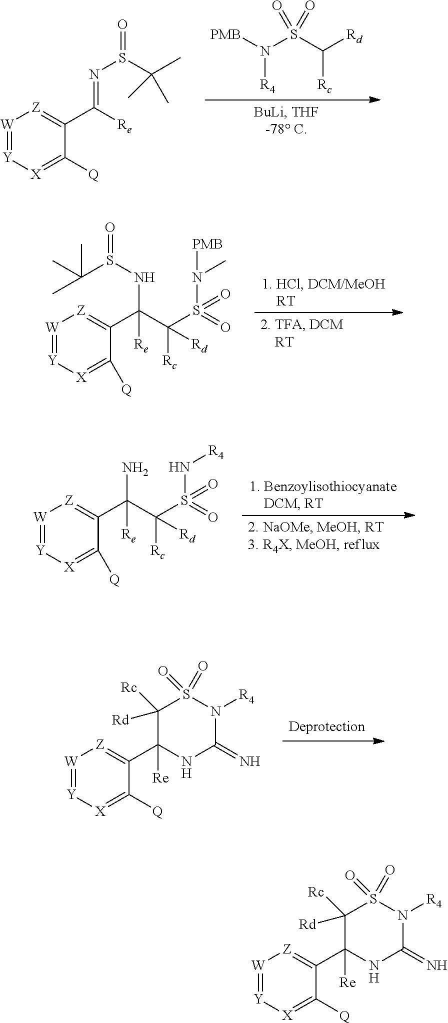

### Step 1

To a flame dried round bottom flask containing a solution of the appropriate PMB protected sulfonamide (2.5 equiv.) in THF (0.3M) at −45° C. under an atmosphere of nitrogen was added dropwise a solution of n-BuLi (2.5 equiv.) and the mixture was stirred for 20 min. After that time, the solution was cooled to −78° C. and transferred by cannula into a precooled solution (−78° C.) of the appropriate sulfinamide intermediate (1 equiv.) in THF (0.1M). The resultant mixture was stirred at −78° C. until completion. The mixture was then quenched with water and the mixture was slowly warmed to RT. The mixture was then extracted with DCM (×3). The combined organic layers were dried over Na₂SO₄, filtered, and concentrated under vacuum. The crude residue was purified by flash chromatography.

### Step 2

To a solution of the above intermediate (1 equiv.) in a mixture of DCM:MeOH (3:1, 0.05M) at RT was added a solution of HCl (4 N in dioxane, 6 eq). The solution was stirred at RT until completion and then the solution was concentrated under vacuum. The resultant residue was re-concentrated under vacuum from toluene (×3) and the mixture was dried further under high vacuum.

### Step 3

To the residue was added DCM (0.5M) followed by TFA (same volume of DCM) and 1,3-dimethoxybenzene (half volume of DCM). The reaction mixture was stirred at RT until completion. After that time, the volatiles were removed under reduced pressure. To the resultant mixture was added 2 M HCl (aq.) and the mixture was extracted with Et₂O (×3). The aqueous layer was then adjusted to ˜pH 10 with the addition of solid Na₂CO₃. The aqueous layer was then extracted with DCM (×3). The combined organic layers were dried over Na₂SO₄, filtered, and concentrated under vacuum. The crude was used for next step without any further purification.

### Steps 4-6

To a solution of the above intermediate (1 equiv.) in DCM (0.15M) was added benzoylisothiocyanate (1.2 equiv.) and the mixture was stirred at RT overnight and the solvent was removed under vacuum. To the residue was added MeOH (0.2M) followed by the addition of a solution of sodium methoxide (25% in MeOH, 2.5 eq). The mixture was stirred at RT for 1 hour after which the solvent was removed in vacuo. The residue was partitioned between EtOAc and water. The aqueous layer was then adjusted to ˜pH 7 with solid NH₄Cl and the mixture was extracted with EtOAc (×3). The combined organic layers were dried over Na₂SO₄, filtered, and concentrated in vacuo to afford the crude thiourea.

To this residue was added EtOH (0.15M) followed by the appropriate electrophile (1.2 equiv.) and the mixture was heated to 70° C. until completion. The mixture was then cooled to RT and the solvent was removed under vacuum. The residue was partitioned between water and DCM and the aqueous layer was basified (pH ˜10) with sat. Na₂CO₃ (aq.). The layers were separated and the aqueous layer was extracted with DCM (×2). The combined organic layers were dried over Na₂SO₄, filtered, and concentrated under vacuum. The crude residue was purified by flash chromatography or prep HPLC.

Step 7—See deprotection steps in General Procedure A of acylguanidine synthesis.

### Synthesis of Acylguanidine Dimers

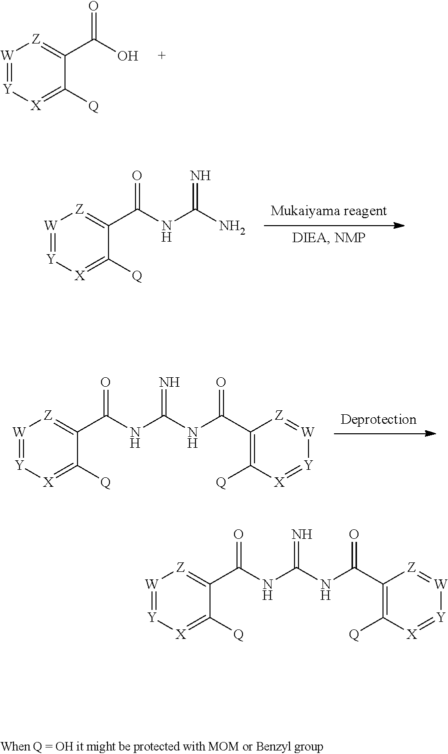

### Step 1

To a solution of the appropriate carboxylic acid (1 equiv.) in NMP (0.2M), DIEA (2 equiv.) and Mukaiyama reagent (1.5 equiv.) were added and reaction stirred at RT for 10-15 min before the appropriate acylguanidine (1.2 equiv.) was added. Reaction stirred at RT until full conversion. Water was added and the mixture was extracted with DCM (×3). The combined organic layers were dried over Na₂SO₄, filtered and concentrated under vacuum. The crude product was purified by prep HPLC.

Step 2—See deprotection steps in General Procedure A of acylguanidine synthesis.

### Synthesis of Aminooxadiazoles

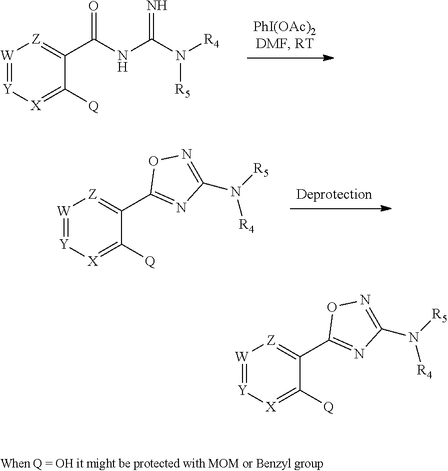

### Step 1

To a solution of the appropriate acylguanidine (1 eq) in DMF (0.2M), PhI(OAc)₂ (1.5 eq) was added and reaction was stirred at RT until full conversion. Water was added and mixture extracted with EtOAc (×3). The combined organic layers were dried over Na₂SO₄, filtered and concentrated under vacuum. The crude product was purified by prep HPLC.

Step 2—See deprotection steps in General Procedure A of acylguanidine synthesis.

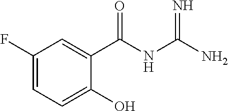 [SMILES: N=C(N)NC(=O)c1cc(F)ccc1O]

This compound was prepared using General Procedure A of acylguanidine synthesis, Method A (LC/MS m/z 198.17 [M+H⁺])

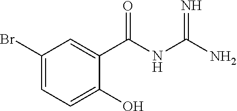 [SMILES: N=C(N)NC(=O)c1cc(Br)ccc1O]

This compound was prepared using General Procedure A of acylguanidine synthesis, Method A (LC/MS m/z 258.09 [M+H⁺])

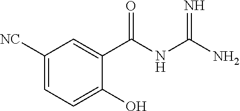 [SMILES: N#Cc1ccc(O)c(C(=O)NC(=N)N)c1]

This compound was prepared using General Procedure A of acylguanidine synthesis, Method A (LC/MS m/z 205.14 [M+H⁺])

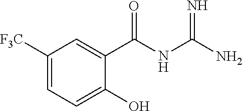 [SMILES: N=C(N)NC(=O)c1cc(C(F)(F)F)ccc1O]

This compound was prepared using General Procedure A of acylguanidine synthesis, Method A (LC/MS m/z 248.16 [M+H⁺])

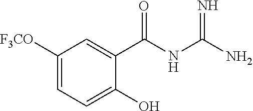 [SMILES: N=C(N)NC(=O)c1cc(OC(F)(F)F)ccc1O]

This compound was prepared using General Procedure A of acylguanidine synthesis, Method A (LC/MS m/z 264.14 [M+H⁺])

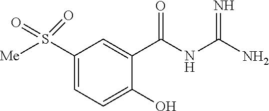 [SMILES: CS(=O)(=O)c1ccc(O)c(C(=O)NC(=N)N)c1]

This compound was prepared using General Procedure A of acylguanidine synthesis, Method A (LC/MS m/z 258.15 [M+H⁺])

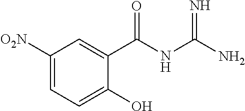 [SMILES: N=C(N)NC(=O)c1cc([N+](=O)[O-])ccc1O]

This compound was prepared using General Procedure A of acylguanidine synthesis, Method A (LC/MS m/z 225.17 [M+H⁺])

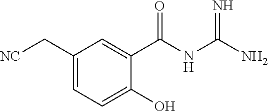 [SMILES: N#CCc1ccc(O)c(C(=O)NC(=N)N)c1]

This compound was prepared using General Procedure A of acylguanidine synthesis, Method A (LC/MS m/z 219.25 [M+H⁺])

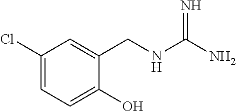 [SMILES: N=C(N)NCc1cc(Cl)ccc1O]

This compound was prepared using General Procedure A of guanidine synthesis (LC/MS m/z 200.18 [M+H⁺])

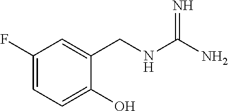 [SMILES: N=C(N)NCc1cc(F)ccc1O]

This compound was prepared using General Procedure A of guanidine synthesis (LC/MS m/z 184.19 [M+H⁺])

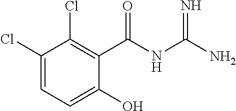 [SMILES: N=C(N)NC(=O)c1c(O)ccc(Cl)c1Cl]

This compound was prepared using General Procedure A of acylguanidine synthesis, Method A (LC/MS m/z 248.16 [M+H⁺])

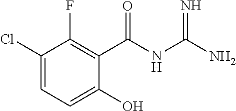 [SMILES: N=C(N)NC(=O)c1c(O)ccc(Cl)c1F]

This compound was prepared using General Procedure A of acylguanidine synthesis, Method A (LC/MS m/z 232.14 [M+H⁺])

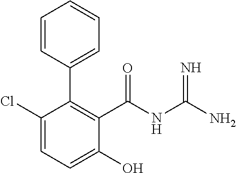 [SMILES: N=C(N)NC(=O)c1c(O)ccc(Cl)c1-c1ccccc1]

This compound was prepared using General Procedure A of acylguanidine synthesis, Method B (LC/MS m/z 290.29 [M+H⁺])

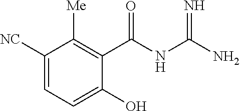 [SMILES: Cc1c(C#N)ccc(O)c1C(=O)NC(=N)N]

This compound was prepared using General Procedure A of acylguanidine synthesis, Method A (LC/MS m/z 219.25 [M+H⁺])

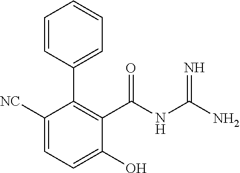 [SMILES: N#Cc1ccc(O)c(C(=O)NC(=N)N)c1-c1ccccc1]

This compound was prepared using General Procedure A of acylguanidine synthesis 1, Method B (LC/MS m/z 281.31 [M+H⁺])

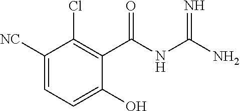 [SMILES: N#Cc1ccc(O)c(C(=O)NC(=N)N)c1Cl]

This compound was prepared using General Procedure A of acylguanidine synthesis, Method B (LC/MS m/z 239.30 [M+H⁺])

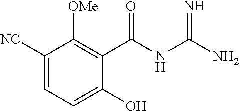 [SMILES: COc1c(C#N)ccc(O)c1C(=O)NC(=N)N]

This compound was prepared using General Procedure A of acylguanidine synthesis, Method B (LC/MS m/z 235.24 [M+H⁺])

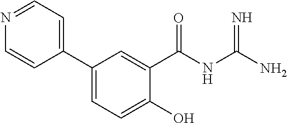 [SMILES: N=C(N)NC(=O)c1cc(-c2ccncc2)ccc1O]

This compound was prepared using General Procedure A of acylguanidine synthesis, Method A (LC/MS m/z 257.24 [M+H⁺])

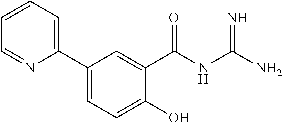 [SMILES: N=C(N)NC(=O)c1cc(-c2ccccn2)ccc1O]

This compound was prepared using General Procedure A of acylguanidine synthesis, Method A (LC/MS m/z 257.27 [M+H⁺])

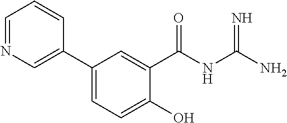 [SMILES: N=C(N)NC(=O)c1cc(-c2cccnc2)ccc1O]

This compound was prepared using General Procedure A of acylguanidine synthesis, Method A (LC/MS m/z 257.24 [M+H⁺])

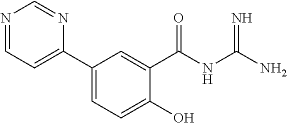 [SMILES: N=C(N)NC(=O)c1cc(-c2ccncn2)ccc1O]

This compound was prepared using General Procedure A of acylguanidine synthesis, Method A (LC/MS m/z 258.32 [M+H⁺])

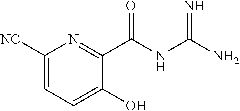 [SMILES: N#Cc1ccc(O)c(C(=O)NC(=N)N)n1]

This compound was prepared using General Procedure A of acylguanidine synthesis, Method A (LC/MS m/z 206.20 [M+H⁺])

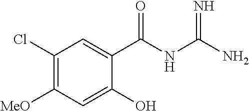 [SMILES: COc1cc(O)c(C(=O)NC(=N)N)cc1Cl]

This compound was prepared using General Procedure A of acylguanidine synthesis, Method A (LC/MS m/z 244.14 [M+H⁺])

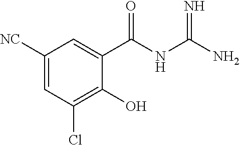 [SMILES: N#Cc1cc(Cl)c(O)c(C(=O)NC(=N)N)c1]

This compound was prepared using General Procedure A of acylguanidine synthesis, Method A (LC/MS m/z 239.18 [M+H⁺])

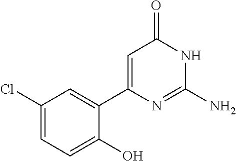 [SMILES: Nc1nc(-c2cc(Cl)ccc2O)cc(=O)[nH]1]

This compound was prepared using General Procedure A of aminopyrimidinone synthesis (LC/MS m/z 238.09 [M+H⁺])

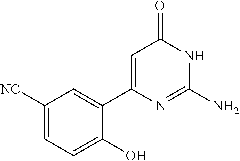 [SMILES: N#Cc1ccc(O)c(-c2cc(=O)[nH]c(N)n2)c1]

This compound was prepared using General Procedure A of aminopyrimidinone synthesis (LC/MS m/z 229.22 [M+H⁺])

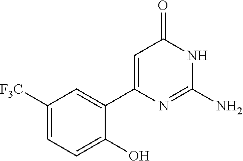 [SMILES: Nc1nc(-c2cc(C(F)(F)F)ccc2O)cc(=O)[nH]1]

This compound was prepared using General Procedure A of aminopyrimidinone synthesis (LC/MS m/z 272.23 [M+H⁺])

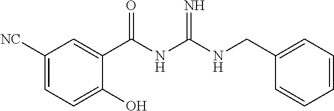 [SMILES: N#Cc1ccc(O)c(C(=O)NC(=N)NCc2ccccc2)c1]

This compound was prepared using General Procedure B of acylguanidine synthesis (LC/MS m/z 295.32 [M+H⁺])

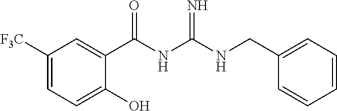 [SMILES: N=C(NCc1ccccc1)NC(=O)c1cc(C(F)(F)F)ccc1O]

This compound was prepared using General Procedure C of acylguanidine synthesis (LC/MS m/z 338.34 [M+H⁺])

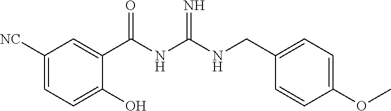 [SMILES: COc1ccc(CNC(=N)NC(=O)c2cc(C#N)ccc2O)cc1]

This compound was prepared using General Procedure B of acylguanidine synthesis (LC/MS m/z 325.35 [M+H⁺])

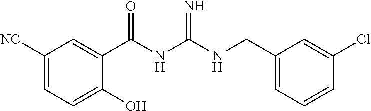 [SMILES: N#Cc1ccc(O)c(C(=O)NC(=N)NCc2cccc(Cl)c2)c1]

This compound was prepared using General Procedure B of acylguanidine synthesis (LC/MS m/z 329.33 [M+H⁺])

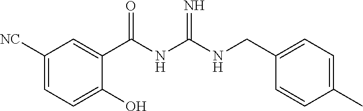 [SMILES: Cc1ccc(CNC(=N)NC(=O)c2cc(C#N)ccc2O)cc1]

This compound was prepared using General Procedure B of acylguanidine synthesis (LC/MS m/z 309.40 [M+H⁺])

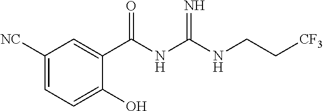 [SMILES: N#Cc1ccc(O)c(C(=O)NC(=N)NCCC(F)(F)F)c1]

This compound was prepared using General Procedure B of acylguanidine synthesis (LC/MS m/z 301.31 [M+H⁺])

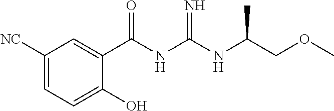 [SMILES: COC[C@H](C)NC(=N)NC(=O)c1cc(C#N)ccc1O]

This compound was prepared using General Procedure B of acylguanidine synthesis (LC/MS m/z 277.33 [M+H⁺])

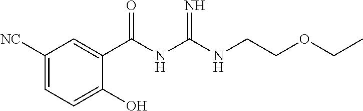 [SMILES: CCOCCNC(=N)NC(=O)c1cc(C#N)ccc1O]

This compound was prepared using General Procedure B of acylguanidine synthesis (LC/MS m/z 277.33 [M+H⁺])

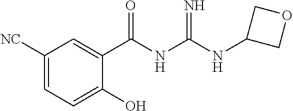 [SMILES: N#Cc1ccc(O)c(C(=O)NC(=N)NC2COC2)c1]

This compound was prepared using General Procedure B of acylguanidine synthesis (LC/MS m/z 261.28 [M+H⁺])

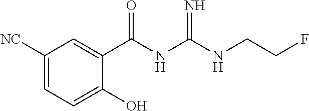 [SMILES: N#Cc1ccc(O)c(C(=O)NC(=N)NCCF)c1]

This compound was prepared using General Procedure B of acylguanidine synthesis (LC/MS m/z 251.29 [M+H⁺])

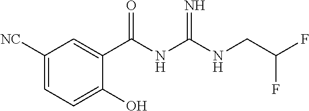 [SMILES: N#Cc1ccc(O)c(C(=O)NC(=N)NCC(F)F)c1]

This compound was prepared using General Procedure B of acylguanidine synthesis (LC/MS m/z 269.27 [M+H⁺])

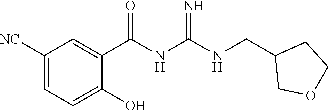 [SMILES: N#Cc1ccc(O)c(C(=O)NC(=N)NCC2CCOC2)c1]

This compound was prepared using General Procedure B of acylguanidine synthesis (LC/MS m/z 289.37 [M+H⁺])

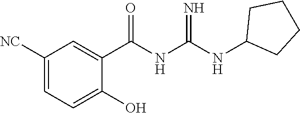 [SMILES: N#Cc1ccc(O)c(C(=O)NC(=N)NC2CCCC2)c1]

This compound was prepared using General Procedure B of acylguanidine synthesis (LC/MS m/z 273.35 [M+H⁺])

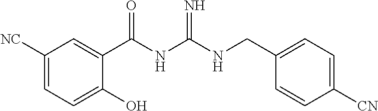 [SMILES: N#Cc1ccc(CNC(=N)NC(=O)c2cc(C#N)ccc2O)cc1]

This compound was prepared using General Procedure B of acylguanidine synthesis (LC/MS m/z 320.38 [M+H⁺])

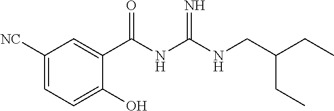 [SMILES: CCC(CC)CNC(=N)NC(=O)c1cc(C#N)ccc1O]

This compound was prepared using General Procedure C of acylguanidine synthesis (LC/MS m/z 289.41 [M+H⁺])

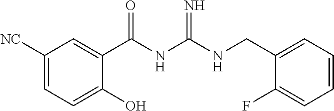 [SMILES: N#Cc1ccc(O)c(C(=O)NC(=N)NCc2ccccc2F)c1]

This compound was prepared using General Procedure C of acylguanidine synthesis (LC/MS m/z 313.38 [M+H⁺])

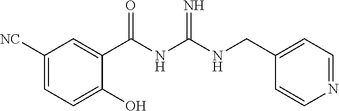 [SMILES: N#Cc1ccc(O)c(C(=O)NC(=N)NCc2ccncc2)c1]

This compound was prepared using General Procedure C of acylguanidine synthesis (LC/MS m/z 296.38 [M+H⁺])

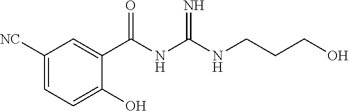 [SMILES: N#Cc1ccc(O)c(C(=O)NC(=N)NCCCO)c1]

This compound was prepared using General Procedure C of acylguanidine synthesis (LC/MS m/z 263.29 [M+H⁺])

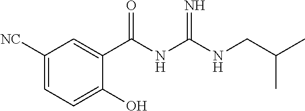 [SMILES: CC(C)CNC(=N)NC(=O)c1cc(C#N)ccc1O]

This compound was prepared using General Procedure B of acylguanidine synthesis (LC/MS m/z 261.33 [M+H⁺])

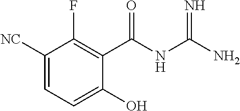 [SMILES: N#Cc1ccc(O)c(C(=O)NC(=N)N)c1F]

This compound was prepared using General Procedure A of acylguanidine synthesis, Method B (LC/MS m/z 223.27 [M+H⁺])

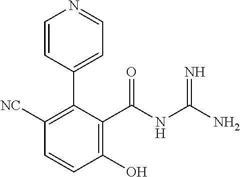 [SMILES: N#Cc1ccc(O)c(C(=O)NC(=N)N)c1-c1ccncc1]

This compound was prepared using General Procedure A of acylguanidine synthesis, Method B (LC/MS m/z 282.33 [M+H⁺])

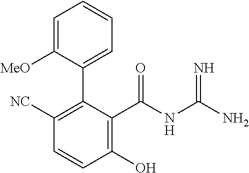 [SMILES: COc1ccccc1-c1c(C#N)ccc(O)c1C(=O)NC(=N)N]

This compound was prepared using General Procedure A of acylguanidine synthesis, Method B (LC/MS m/z 311.34 [M+H⁺])

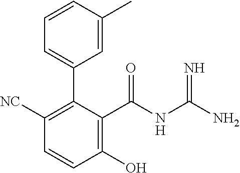 [SMILES: Cc1cccc(-c2c(C#N)ccc(O)c2C(=O)NC(=N)N)c1]

This compound was prepared using General Procedure A of acylguanidine synthesis, Method B (LC/MS m/z 295.36 [M+H⁺])

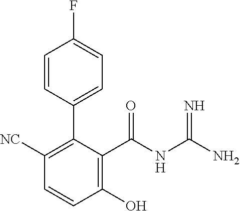 [SMILES: N#Cc1ccc(O)c(C(=O)NC(=N)N)c1-c1ccc(F)cc1]

This compound was prepared using General Procedure A of acylguanidine synthesis, Method B (LC/MS m/z 299.37 [M+H⁺])

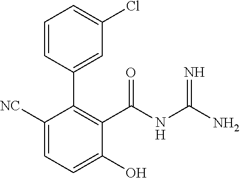 [SMILES: N#Cc1ccc(O)c(C(=O)NC(=N)N)c1-c1cccc(Cl)c1]

This compound was prepared using General Procedure A of acylguanidine synthesis, Method B (LC/MS m/z 315.35 [M+H⁺])

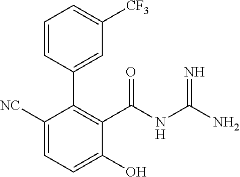 [SMILES: N#Cc1ccc(O)c(C(=O)NC(=N)N)c1-c1cccc(C(F)(F)F)c1]

This compound was prepared using General Procedure A of acylguanidine synthesis, Method B (LC/MS m/z 349.36 [M+H⁺])

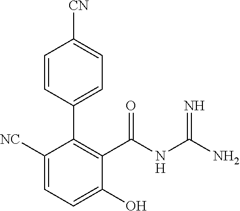 [SMILES: N#Cc1ccc(-c2c(C#N)ccc(O)c2C(=O)NC(=N)N)cc1]

This compound was prepared using General Procedure A of acylguanidine synthesis, Method B (LC/MS m/z 306.34 [M+H⁺])

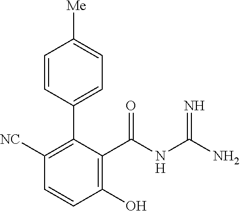 [SMILES: Cc1ccc(-c2c(C#N)ccc(O)c2C(=O)NC(=N)N)cc1]

This compound was prepared using General Procedure A of acylguanidine synthesis, Method B (LC/MS m/z 295.32 [M+H⁺])

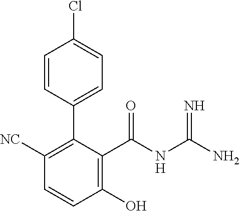 [SMILES: N#Cc1ccc(O)c(C(=O)NC(=N)N)c1-c1ccc(Cl)cc1]

This compound was prepared using General Procedure A of acylguanidine synthesis, Method B (LC/MS m/z 315.32 [M+H⁺])

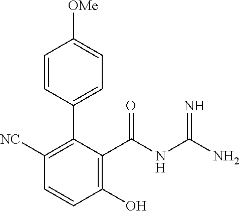 [SMILES: COc1ccc(-c2c(C#N)ccc(O)c2C(=O)NC(=N)N)cc1]

This compound was prepared using General Procedure A of acylguanidine synthesis, Method B (LC/MS m/z 311.30 [M+H⁺])

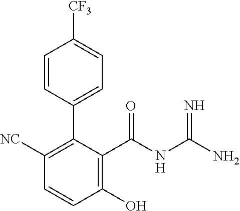 [SMILES: N#Cc1ccc(O)c(C(=O)NC(=N)N)c1-c1ccc(C(F)(F)F)cc1]

This compound was prepared using General Procedure A of acylguanidine synthesis, Method B (LC/MS m/z 349.32 [M+H⁺])

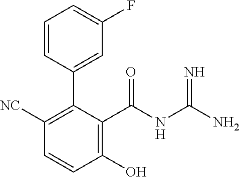 [SMILES: N#Cc1ccc(O)c(C(=O)NC(=N)N)c1-c1cccc(F)c1]

This compound was prepared using General Procedure A of acylguanidine synthesis, Method B (LC/MS m/z 299.30 [M+H⁺])

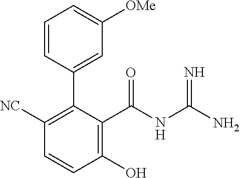 [SMILES: COc1cccc(-c2c(C#N)ccc(O)c2C(=O)NC(=N)N)c1]

This compound was prepared using General Procedure A of acylguanidine synthesis, Method B (LC/MS m/z 311.37 [M+H⁺])

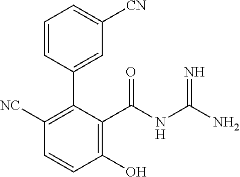 [SMILES: N#Cc1cccc(-c2c(C#N)ccc(O)c2C(=O)NC(=N)N)c1]

This compound was prepared using General Procedure A of acylguanidine synthesis, Method B (LC/MS m/z 306.37 [M+H⁺])

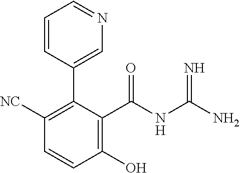 [SMILES: N#Cc1ccc(O)c(C(=O)NC(=N)N)c1-c1cccnc1]

This compound was prepared using General Procedure A of acylguanidine synthesis, Method B (LC/MS m/z 282.30 [M+H⁺])

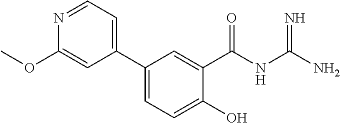 [SMILES: COc1cc(-c2ccc(O)c(C(=O)NC(=N)N)c2)ccn1]

This compound was prepared using General Procedure A of acylguanidine synthesis, Method A (LC/MS m/z 287.40 [M+H³⁰]).

### Example 4: General Procedures for the Synthesis of Representative Compounds of the Invention

### General Procedure 1

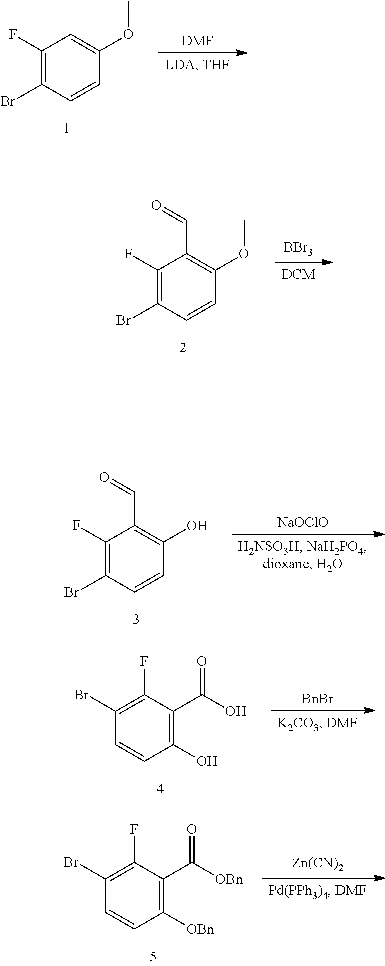

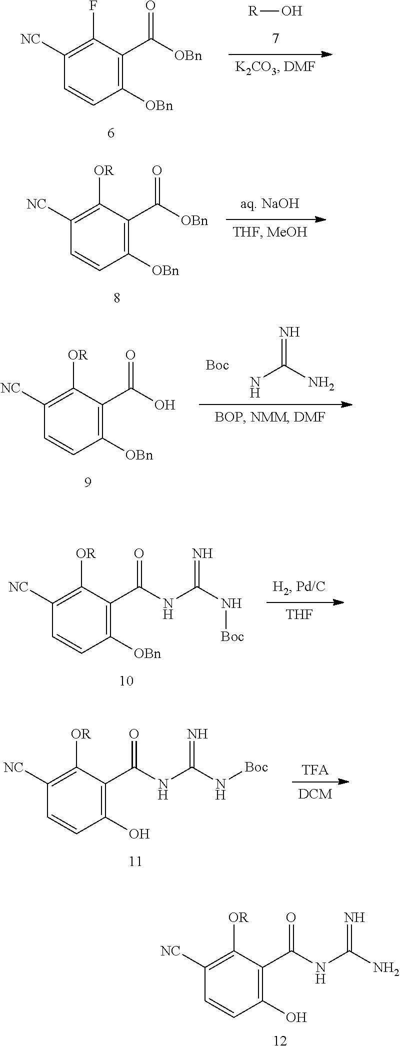

### Step 1: Synthesis of 3-bromo-2-fluoro-6-methoxybenzaldehyde (2)

LDA (73 mL, 2 M in THF) was added dropwise to a solution of compound 1 (25 g, 121.94 mmol) in THF (250 mL) at −78° C. under N₂ atmosphere. The mixture was stirred at −78° C. for 1 hour before anhydrous DMF (10.69 g, 146.32 mmol) was added. The resulting mixture was stirred at −78° C. for additional 30 minutes. The mixture was then diluted with ice/H₂O and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=20:1 to 5:1) to give compound 2 (23.0 g, 80.94% yield) as a yellow solid. LC/MS (ESI) m/z: 233/235 (M+H)⁺.

### Step 2: Synthesis of 3-bromo-2-fluoro-6-hydroxybenzaldehyde (3)

BBr₃ (25.80 g, 102.99 mmol) was added dropwise to a solution of 3-bromo-2-fluoro-6-methoxybenzaldehyde (12 g, 51.49 mmol) in anhydrous DCM (120 mL) at −50° C. under N₂ atmosphere. The resulting mixture was slowly warmed to room temperature and stirred for 1 hour. The mixture was then quenched with ice/H₂O and extracted with DCM twice. The combined organic layers were washed with brine, dried over anhydrous Na₂SO₄, filtered and concentrated under vacuum to give compound 3 (10.37 g, 91.98% yield) as a yellow solid. LC/MS (ESI) m/z: 217/219 (M−H)⁻.

### Step 3: Synthesis of 3-bromo-2-fluoro-6-hydroxybenzoic acid (4)

H₂NSO₃H (6.89 g, 71.05 mmol) and NaH₂PO₄ (22.16 g, 184.72 mmol) were added to a solution of crude 3-bromo-2-fluoro-6-hydroxybenzaldehyde (10.37 g, 47.36 mmol) in dioxane (100 mL) at 0° C. followed by a solution of NaOClO (5.57 g, 61.57 mmol) in H₂O (100 mL) dropwise. The resulting mixture was stirred for 30 minutes at 0° C. and then diluted with H₂O and extracted with EtOAc twice. The combined organic layers were washed with brine, dried over anhydrous Na₂SO₄, filtered and concentrated under vacuum to give compound 4 (11.10 g, 99.72% yield) as a yellow solid. LC/MS (ESI) m/z: 233/235 (M−H)⁻.

### Step 4: Synthesis of benzyl 6-(benzyloxy)-3-bromo-2-fluorobenzoate (5)

Potassium carbonate (32.59 g, 236.16 mmol) and benzylbromide (24.23 g, 141.70 mmol) were added to a solution of crude 3-bromo-2-fluoro-6-hydroxybenzoic acid (11.10 g, 47.23 mmol) in DMF (100 mL) at 0° C. The resulting mixture was stirred at room temperature for 16 hours. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by column chromatography on silica gel (Petroleum Ether:EtOAc=80:1 to 40:1) to give compound 5 (12.14 g, 61.92% yield) as a yellow solid. LC/MS (ESI) m/z: 415/417 (M+H)⁺.

Step 5: Synthesis of benzyl 6-(benzyloxy)-3-cyano-2-fluorobenzoate (6) Zinc cyanide (0.51 g, 4.33 mmol) and Pd(PPh₃)₄ (0.42 g, 0.36 mmol) were added to a solution of 6-(benzyloxy)-3-bromo-2-fluorobenzoate (1.5 g, 3.61 mmol) in DMF (10 mL) under N₂ atmosphere. The resulting mixture was stirred at 135° C. for 30 minutes under microwave. The mixture was then diluted with EtOAc and filtered. The filtrate was washed with water, saturated aq. NH₄Cl solution and brine, dried over anhydrous Na₂SO₄, filtered and concentrated under vacuum. The residue was purified by column chromatography on silica gel (Petroleum Ether:pEtOAc=30:1 to 10:1) to give compound 6 (0.5 g, 38.30% yield) as a light yellow solid. LC/MS (ESI) m/z: 362 (M+H)⁺.

### Step 6: Synthesis of Compound 8

Potassium carbonate (5.0 eq) was added to a solution of benzyl 6-(benzyloxy)-3-cyano-2-fluorobenzoate (1.0 eq) in DMF followed by the appropriate phenol (3.0 eq). The resulting mixture was stirred at room temperature for 16 hours. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by column chromatography on silica gel (Petroleum Ether:pEtOAc=30:1 to 10:1) to give compound 8.

### Step 7: Synthesis of Compound 9

Aqueous. NaOH (2 M, 8.0 eq) was added to a solution of compound 8 (1.0 eq) in MeOH/THF (1:1, v/v) at 0° C. The reaction mixture was stirred at 70° C. overnight. The mixture was then cooled to room temperature and washed with Et₂O. The aqueous layer was separated, acidified to pH=5 with 0.5 M aq. HCl solution and extracted with EtOAc twice. The organic layers were washed with brine, dried over anhydrous Na₂SO₄, filtered and concentrated under vacuum to give compound 9.

### Step 8: Synthesis of Compound 10

NMM (4.0 eq) and PyBOP (1.4 eq) were added to a mixture of compound 9 (1.0 eq) and Boc-guanidine (2.6 eq) in DMF, and the resulting mixture was stirred at room temperature overnight under N₂ atmosphere. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by column chromatography on silica gel (Petroleum Ether:EtOAc=10:1 to 4:1) to give compound 10.

### Step 9: Synthesis of Compound 11

10% Pd/C (1.0 eq) was added to a solution of compound 10 (1.0 eq) in THF and the mixture was degassed under N₂ atmosphere for three times and stirred under H₂ atmosphere at room temperature for 30 minutes. The mixture was then filtered and the filtrate was concentrated under vacuum. The residue was purified by column chromatography on silica gel (Petroleum Ether:pEtOAc=10:1 to 5:1) to give compound 11.

### Step 10: Synthesis of Compound 12

TFA (1 ml) was added to a solution of compound 11 (1.0 eq) in DCM (1 mL) at 0° C. and the reaction was stirred at room temperature for 1 hour. The mixture was then concentrated under vacuum and the residue was purified via prep-HPLC (C18, 0% to 50% acetonitrile in H₂O with 0.1% NH₃.H₂O) to give the final compound 12.

### Synthesis of A69

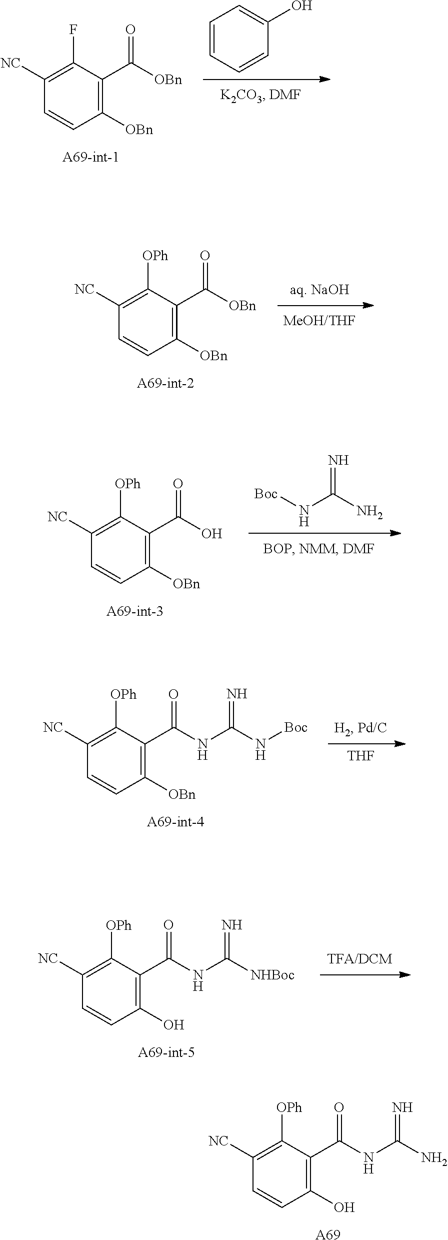

### Step 1: Synthesis of Compound A69-Int-2

Potassium carbonate (592 mg, 4.29 mmol) was added to a mixture of compound A69-int-1 (310 mg, 0.86 mmol) and phenol (242.21 mg, 2.57 mmol) in DMF (5 mL). The resulting mixture was stirred at room temperature overnight. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by column chromatography on silica gel (Petroleum Ether:EtOAc=30:1 to 10:1) to give compound A69-int-2 (356 mg, 95.29% yield) as a yellow solid. LC/MS (ESI) m/z: 436 (M+H)⁺.

### Step 2: Synthesis of Compound A69-Int-3

A solution of NaOH (196 mg, 4.91 mmol) in H₂O (10 mL) was added to a solution of compound A69-int-2 (356 mg, 0.82 mmol) in MeOH (10 mL) and THF (10 mL) at 0° C. The mixture was stirred at 70° C. overnight. The mixture was then washed with Et₂O and the aqueous layer was separated, acidified with 0.5 M aq. HCl solution to pH=5 and extracted with EtOAc twice. The combined organic layers were washed with brine, dried over anhydrous Na₂SO₄, filtered and concentrated under vacuum to give compound A69-int-3 (280 mg, 99.18% yield) as a yellow solid without any further purification. LC/MS (ESI) m/z: 344 (M−H)⁻.

### Step 3: Synthesis of Compound A69-Int-4

NMM (491 mg, 4.86 mmol) and PyBOP (573 mg, 1.30 mmol) were added to a mixture of compound A69-int-3 (280 mg, 0.81 mmol) and Boc-guanidine (335 mg, 2.11 mmol) in DMF (4 mL). The resulting mixture was stirred at room temperature overnight under N₂ atmosphere. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by column chromatography on silica gel (Petroleum Ether:EtOAc=10:1 to 4:1) to give compound A69-int-4 (390 mg, 98.87% yield) as a white solid. LC/MS (ESI) m/z: 487 (M+H)⁺.

### Step 4: Synthesis of Compound A69-Int-5

10% Pd/C (210 mg) was added to a solution of compound A69-int-4 (210 mg, 0.43 mmol) in THF (8 mL). The mixture was degassed under N₂ atmosphere for three times and stirred under 20 psi H₂ at room temperature for 30 minutes. The mixture was then filtered and the filtrate was concentrated under vacuum. The residue was purified by column chromatography on silica gel (Petroleum Ether:EtOAc=10:1 to 5:1) to give compound A69-int-5 (126 mg, 73.65% yield) as a white solid. LC/MS (ESI) m/z: 397 (M+H)⁺.

### Step 5: Synthesis of Compound A69

TFA (3 ml) was added dropwise to a solution of compound A69-int-5 (126 mg, 0.32 mmol) in DCM (3 mL) at 0° C. The reaction mixture was stirred at room temperature for 1 hour and then concentrated under vacuum. The residue was purified by prep-HPLC (C18, 0% to 50% acetonitrile in H₂O with 0.1% NH₃.H₂O) to give compound A69 (23.7 mg, 25.16% yield) as a white solid. LC/MS (ESI) m/z: 297 (M+H)⁺. ¹H NMR (400 MHz, DMSO-d₆) δ 8.15 (br s, 2H), 7.56 (d, J=8.9 Hz, 1H), 7.27 (t, J=7.8 Hz, 2H), 6.97 (t, J=7.1 Hz, 1H), 6.75 (d, J=8.5 Hz, 2H), 6.67 (d, J=8.9 Hz, 1H).

The compounds in the table below were prepared from the appropriate starting materials described previously or commercially available using the above general procedure 1.

| Structure | Name |
|---|---|
| 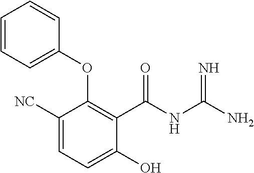 [SMILES: N#Cc1ccc(O)c(C(=O)NC(=N)N)c1Oc1ccccc1] | N-carbamimidoyl-3-cyano-6-hydroxy-2- phenoxybenzamide LC-MS: m/z 297 (M + H)⁺. 1H NMR (400 MHz, DMSO) δ 8.15 (br, 2H), 7.56 (d, J = 8.9 Hz, 1H), 7.27 (t, J = 7.8 Hz, 2H), 6.97 (t, J = 7.1 Hz, 1H), 6.75 (d, J = 8.5 Hz, 2H), 6.67 (d, J = 8.9 Hz, 1H). |
| 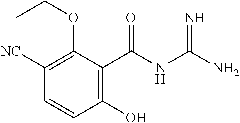 [SMILES: CCOc1c(C#N)ccc(O)c1C(=O)NC(=N)N] | N-carbamimidoyl-3-cyano-2-ethoxy-6- hydroxybenzamide LC-MS: m/z 249 (M + H)⁺. 1H NMR (400 MHz, DMSO) δ 8.49 (br, 2H), 7.35 (d, J = 8.8 Hz, 1H), 6.42 (d, J = 8.8 Hz, 1H), 4.02 (q, J = 7.0 Hz, 2H), 1.31 (t, J = 7.0 Hz, 3H). |
| 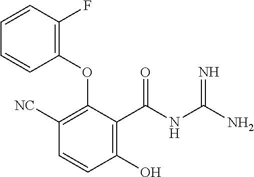 [SMILES: N#Cc1ccc(O)c(C(=O)NC(=N)N)c1Oc1ccccc1F] | N-carbamimidoyl-3-cyano-2-(2- fluorophenoxy)-6-hydroxybenzamide LC-MS: m/z 315 (M + H)⁺. 1H NMR (400 MHz, DMSO) δ 8.44 (br s, 2H), 7.60 (d, J = 8.9 Hz, 1H), 7.32 (br s, 1H) 7.30-7.20 (m, 1H), 7.03-6.95 (m, 2H), 6.71 (d, J = 8.9 Hz, 1H), 6.58-6.52 (m, 1H). |
| 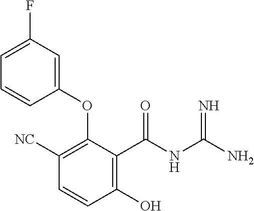 [SMILES: N#Cc1ccc(O)c(C(=O)NC(=N)N)c1Oc1cccc(F)c1] | N-carbamimidoyl-3-cyano-2-(3- fluorophenoxy)-6-hydroxybenzamide LC-MS: m/z 315 (M + H)⁺. 1H NMR (400 MHz, DMSO) δ 8.45 (br s, 2H), 7.60 (d, J = 8.9 Hz, 1H), 7.38 (br s, 1H), 7.28-7.17 (m, 1H), 6.83 (td, J = 8.3, 2.1 Hz, 1H), 6.71 (d, J = 8.9 Hz, 1H), 6.65 (dt, J = 10.7, 2.4 Hz, 1H), 6.57 (dd, J = 8.2, 2.2 Hz, 1H). |
| 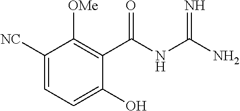 [SMILES: COc1c(C#N)ccc(O)c1C(=O)NC(=N)N] | N-carbamimidoyl-3-cyano-6-hydroxy-2- methoxybenzamide LC-MS: m/z 235 (M + H)⁺. 1H NMR (400 MHz, DMSO) δ 7.96 (br s, 3H), 7.46 (d, J = 8.8 Hz, 1H), 6.55 (d, J = 8.8 Hz, 1H), 3.82 (s, 3H). |

### General Procedure 2

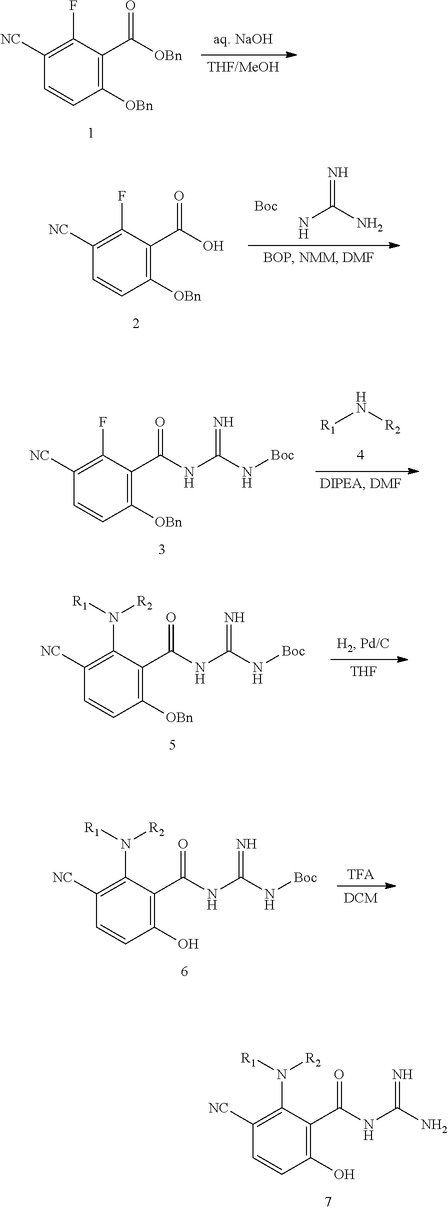

### Step 1: Synthesis of Compound 2

A solution of NaOH (521 mg, 13.03 mmol) in H₂O (10 mL) was added at 0° C. to a solution of compound 1 (942 mg, 2.61 mmol) in MeOH (10 mL) and THF (10 mL). The mixture was stirred at room temperature for 16 hours and then washed with Et₂O. The aqueous layer was acidified with 0.5M aq. HCl solution and extracted with EtOAc twice. The organic layers were washed with brine, dried over anhydrous Na₂SO₄, filtered and concentrated under vacuum to give compound 2 (648 mg, 91.65% yield) as a yellow solid without any further purification. LC/MS (ESI) m/z: 270 (M−H)⁻.

### Step 2: Synthesis of Compound 3

NMM (1448 mg, 14.33 mmol) was added followed by PyBOP (1690 mg, 3.82 mmol) to a mixture of compound 2 (648 mg, 2.39 mmol) and Boc-guanidine (988 mg, 6.21 mmol) in DMF (10 mL). The resulting mixture was stirred at room temperature for 16 hours. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by column chromatography on silica gel (Petroleum Ether:EtOAc=10:1 to 3:1) to give compound 3 (550 mg, 55.82% yield) as a white solid. LC/MS (ESI) m/z: 413 (M+H)⁺.

### Step 3: Synthesis of Compound 5

The appropriate amine 4 (6.0 eq) was added to a solution of compound 3 (1.0 eq) and DIPEA (8.0 eq) in DMF and the resulting mixture was stirred at room temperature for 40 hours. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified via prep-TLC to give compound 5.

### Step 4: Synthesis of Compound 6

10% Pd/C (1.0 eq) was added to a solution of compound 5 (1.0 eq) in THF, and the mixture was degassed under N₂ atmosphere for three times and stirred under H₂ atmosphere at room temperature for 30 minutes. The mixture was then filtered and the filtrate was concentrated under vacuum. The residue was purified by prep-TLC to give compound 6.

### Step 5: Synthesis of Compound 7

TFA (1 ml) was added dropwise at 0° C. to a solution of compound 6 (1.0 eq) in DCM (1 mL) and the reaction was stirred at room temperature for 1 hour. The mixture was then concentrated under vacuum and the residue was purified by prep-HPLC (C18, 0% to 50% acetonitrile in H₂O with 0.1% NH₃.H₂O) to give compound 7.

### Synthesis of A124

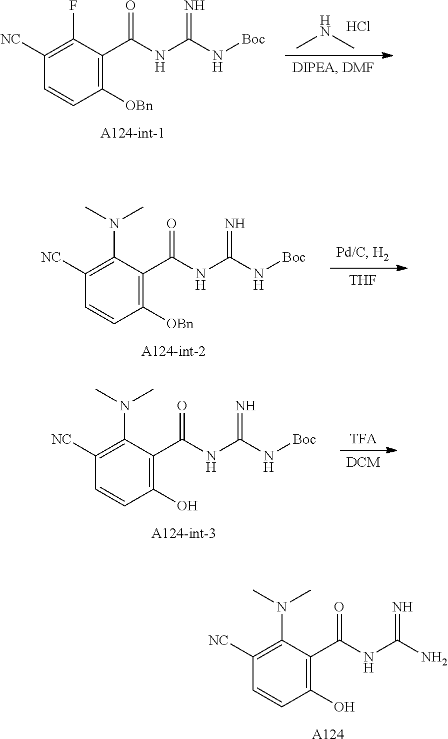

### Step 1: Synthesis of Compound A124-Int-2

Dimethylamine hydrochloride (178 mg, 2.18 mmol) was added to a mixture of compound A124-int-1 (150 mg, 0.36 mmol) and DIPEA (375 mg, 2.91 mmol) in DMF (3 mL). The resulting mixture was stirred at room temperature for 40 hours. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by prep-TLC to give compound A124-int-2 (70 mg, 43.99% yield) as a white solid. LC/MS (ESI) m/z: 438 (M+H)⁺.

### Step 2: Synthesis of Compound A124-Int-3

10% Pd/C (70 mg) was added to a solution of compound A124-int-2 (70 mg, 0.16 mmol) in THF (5 mL), and the mixture was degassed under N₂ atmosphere for three times and stirred under 15 psi H₂ at room temperature for 30 minutes. The mixture was then filtered and the filtrate was concentrated under vacuum. The residue was purified by prep-TLC to give compound A124-int-3 (30 mg, 53.98% yield) as a yellow solid. LC/MS (ESI) m/z: 348 (M+H)⁺.

### Step 3: Synthesis of A124

TFA (3 ml) was added dropwise at 0° C. to a solution of compound A124-int-3 (30 mg, 0.09 mmol) in DCM (3 mL) and the reaction was stirred at room temperature for 2 hours. The mixture was then concentrated, diluted with saturated aq. NaHCO₃ and extracted with EtOAc twice. The combined organic layers were washed with brine, dried over anhydrous Na₂SO₄, filtered and concentrated under vacuum. The residue was purified via prep-HPLC (C18, 0% to 50% acetonitrile in H₂O with 0.1% NH₃.H₂O) to give compound A124 (5.5 mg, 25.75% yield) as a light yellow solid. LC/MS (ESI) m/z: 248 (M+H)⁺. ¹H NMR (400 MHz, DMSO-d₆) δ 7.95 (s, 3H), 7.38 (d, J=8.6 Hz, 1H), 6.40 (d, J=8.6 Hz, 1H), 2.86 (s, 6H).

The compounds in the table below were prepared from the appropriate starting materials described previously or commercially available using the above general procedure 2.

| Structure | Name |
|---|---|
| 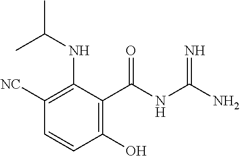 [SMILES: CC(C)Nc1c(C#N)ccc(O)c1C(=O)NC(=N)N] | N-carbamimidoyl-3-cyano-6-hydroxy-2- (isopropylamino)benzamide LC-MS: m/z 262 (M + H)⁺. 1H NMR (400 MHz, DMSO) δ 9.78 (d, J = 8.3 Hz, 1H), 8.28 (br s, 2H), 7.25 (br s, 1H), 7.22 (d, J = 8.8 Hz, 1H), 6.01 (d, J = 8.8 Hz, 1H), 4.47-4.33 (m, 1H), 1.20 (d, J = 6.2 Hz, 6H). |
| 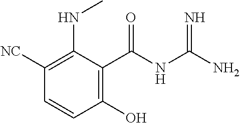 [SMILES: CNc1c(C#N)ccc(O)c1C(=O)NC(=N)N] | N-carbamimidoyl-3-cyano-6-hydroxy-2- (methylamino)benzamide LC-MS: m/z 234 (M + H)⁺. 1H NMR (400 MHz, DMSO) δ 9.80 (q, J = 4.8 Hz, 1H), 8.27 (br s, 2H), 7.26 (br s, 1H), 7.23 (d, J = 8.8 Hz, 1H), 6.00 (d, J = 8.8 Hz, 1H), 3.17 (d, J = 5.2 Hz, 3H). |
| 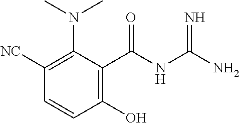 [SMILES: CN(C)c1c(C#N)ccc(O)c1C(=O)NC(=N)N] | N-carbamimidoyl-3-cyano-2-(dimethylamino)- 6-hydroxybenzamide LC-MS: m/z 248 (M + H)⁺. 1H NMR (400 MHz, DMSO) δ 7.95 (br s, 3H), 7.38 (d, J = 8.6 Hz, 1H), 6.40 (d, J = 8.6 Hz, 1H), 2.86 (s, 6H). |
| 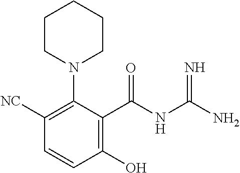 [SMILES: N#Cc1ccc(O)c(C(=O)NC(=N)N)c1N1CCCCC1] | N-carbamimidoyl-3-cyano-6-hydroxy-2- (piperidin-1-yl)benzamide LC-MS: m/z 288 (M + H)⁺. 1H NMR (400 MHz, DMSO) δ 15.00 (s, 1H), 8.32 (br s, 2H), 7.42 (d, J = 8.6 Hz, 1H), 7.13 (br s, 1H), 6.43 (d, J = 8.6 Hz, 1H), 3.15-3.06 (m,4H), 1.66-1.52 (m, 6H). |

### General Procedure 3

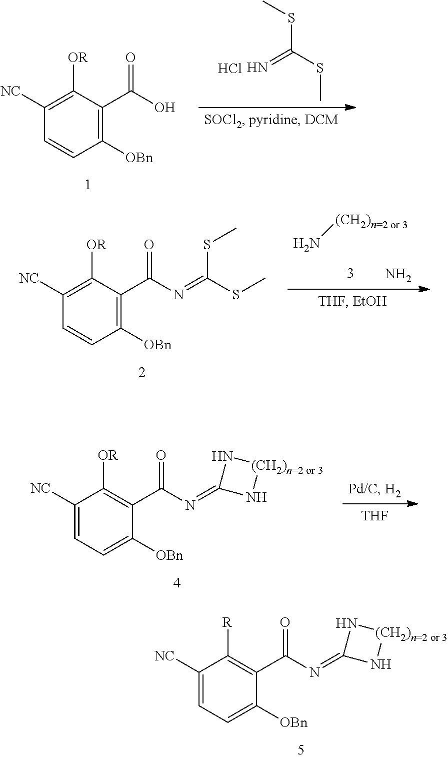

### Step 1: Synthesis of Compound 2

Thionyl chloride (10.0 eq) was added at 0° C. to a solution of compound 1 (1.0 eq) in DCM, and the reaction was stirred at 60° C. for 2 hours. The mixture was then cooled to room temperature and concentrated under vacuum. The crude residue was added to a mixture of dimethyl carbonimidodithioate (1.5 eq) and pyridine (1.5 eq) in DCM at 0° C. and the resulting mixture was stirred at room temperature for 30 minutes. The mixture was then concentrated under vacuum and the residue was purified by column chromatography on silica gel (Petroleum Ether:pEtOAc=10:1 to 5:1) to give compound 2.

### Step 2: Synthesis of Compound 4

The appropriate diamine 3 (2.0 eq) was added to a solution of compound 2 (1.0 eq) in THF/EtOH (1:1, v/v) and the resulting mixture was stirred at 80° C. for 1 hour. The mixture was then cooled to room temperature and concentrated under vacuum to give a residue that was purified by column chromatography on silica gel (DCM:MeOH=80:1 to 40:1) to give compound 4.

### Step 3: Synthesis of Compound 5

10% Pd/C (1.0 eq) was added to a solution of compound 4 (1.0 eq) in THF and the mixture was degassed under N₂ atmosphere for three times and stirred under H₂ atmosphere at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to give compound 5.

### Synthesis of A179

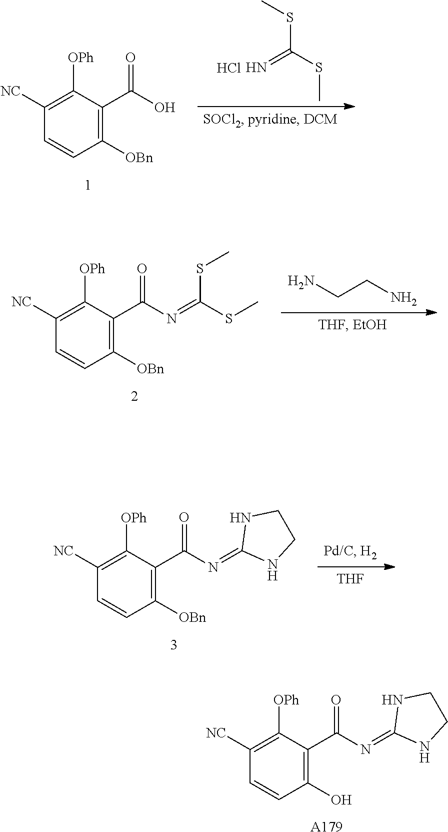

### Step 1: Synthesis of Compound 2

Thionyl chloride (0.40 mL, 5.50 mmol) was added at 0° C. to a solution of compound 1 (190 mg, 0.55 mmol) in DCM (5 mL). The reaction was stirred at 60° C. for 2 hours. The mixture was then cooled to room temperature and concentrated under vacuum. The crude residue was added to a mixture of dimethyl carbonimidodithioate (100 mg, 0.83 mmol) and pyridine (65 mg, 0.83 mmol) in DCM (5 mL) at 0° C. and the resulting mixture was stirred at room temperature for 30 minutes. The mixture was then concentrated under vacuum and the residue was purified by column chromatography on silica gel (Petroleum Ether:EtOAc=10:1 to 5:1) to give compound 2 (160 mg, 64.83% yield) as a white solid. LC/MS (ESI) m/z: 449 (M+H)⁺.

### Step 2: Synthesis of Compound 3

Ethylenediamine (0.02 mL, 0.36 mmol) was added to a solution of compound 2 (80 mg, 0.18 mmol) in THF (4 mL) and EtOH (4 mL) and the mixture was stirred at 80° C. for 1 hour. The mixture was then cooled to room temperature and concentrated under vacuum. The residue was purified by column chromatography on silica gel (DCM:MeOH=80:1 to 40:1) to give compound 3 (70 mg, 95.19% yield) as a white solid. LC/MS (ESI) m/z: 413 (M+H)⁺.

### Step 3: Synthesis of Compound A179

10% Pd/C (70 mg) was added to a solution of compound 3 (70 mg, 0.17 mmol) in THF (4 mL) and the mixture was degassed under N₂ atmosphere for three times and stirred under 15 psi H₂ at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to give compound A179 (19.1 mg, 34.92% yield) as a white solid. LC/MS (ESI) m/z: 323 (M+H)⁺. ¹H NMR (400 MHz, DMSO-d₆) δ 8.68 (s, 2H), 7.67 (d, J=8.8 Hz, 1H), 7.28 (t, J=8.0 Hz, 2H), 6.98 (t, J=7.3 Hz, 1H), 6.77 (dd, J=18.1, 8.4 Hz, 3H), 3.54 (s, 4H).

The compounds in the table below were prepared from the appropriate starting materials described previously or commercially available using the above general procedure 3.

| Structure | Name |
|---|---|
| 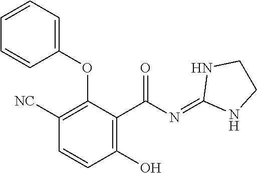 [SMILES: N#Cc1ccc(O)c(C(=O)N=C2NCCN2)c1Oc1ccccc1] | 3-cyano-6-hydroxy-N-(imidazolidin-2- ylidene)-2-phenoxybenzamide LC-MS: m/z 323 (M + H)⁺. 1H NMR (400 MHz, DMSO) δ 8.68 (s, 2H), 7.67 (d, J = 8.8 Hz, 1H), 7.28 (t, J = 8.0 Hz, 2H), 6.98 (t, J = 7.3 Hz, 1H), 6.81-6.74 (m, 3H), 3.54 (s, 4H). |
| 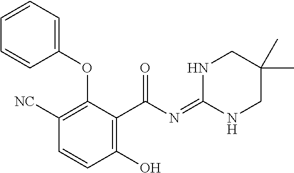 [SMILES: CC1(C)CNC(=NC(=O)c2c(O)ccc(C#N)c2Oc2ccccc2)NC1] | 3-cyano-N-(5,5-dimethyltetrahydropyrimidin- 2(1H)-ylidene)-6-hydroxy-2- phenoxybenzamide LC-MS: m/z 365 (M + H)⁺. 1H NMR (400 MHz, DMSO) δ 9.10 (s, 2H), 7.61 (d, J = 8.9 Hz, 1H), 7.28 (t, J = 8.0 Hz, 2H), 6.98 (t, J = 7.3 Hz, 1H), 6.77-6.71 (m, 3H), 2.97 (s, 4H), 0.95 (s, 6H). |
| 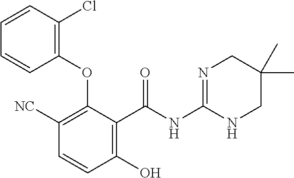 [SMILES: CC1(C)CN=C(NC(=O)c2c(O)ccc(C#N)c2Oc2ccccc2Cl)NC1] | 2-(2-chlorophenoxy)-3-cyano-N-(5,5 - dimethyl-1,4,5,6-tetrahydropyrimidin-2-yl)-6- hydroxybenzamide LC-MS: m/z 399 (M + H)⁺. 1H NMR (400 MHz, DMSO) δ 9.11 (s, 2H), 7.63 (d, J = 8.8 Hz, 1H), 7.49 (dd, J = 8.0, 1.6 Hz, 1H), 7.22-7.13 (m, 1H), 7.00 (td, J = 7.6, 1.2 Hz, 1H), 6.76 (d, J = 8.8 Hz, 1H), 6.51 (d, J = 7.2 Hz, 1H), 2.98 (s, 4H), 0.95 (s, 6H). |
| 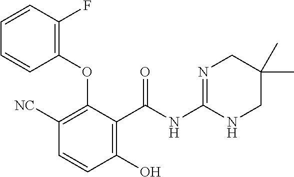 [SMILES: CC1(C)CN=C(NC(=O)c2c(O)ccc(C#N)c2Oc2ccccc2F)NC1] | 3-cyano-N-(5,5-dimethyl-1,4,5,6- tetrahydropyrimidin-2-yl)-2-(2- fluorophenoxy)-6-hydroxybenzamide LC-MS: m/z 383 (M + H)⁺. 1H NMR (400 MHz, MeOD) δ 7.47 (dd, J = 8.9, 3.2 Hz, 1H), 7.17-7.09 (m, 1H), 6.99- 6.91 (m, 2H), 6.71 (dd, J = 8.9, 3.0 Hz, 1H), 6.62-6.57 (m, 1H), 3.04 (s, 4H), 1.04 (s, 6H). |

### General Procedure 4

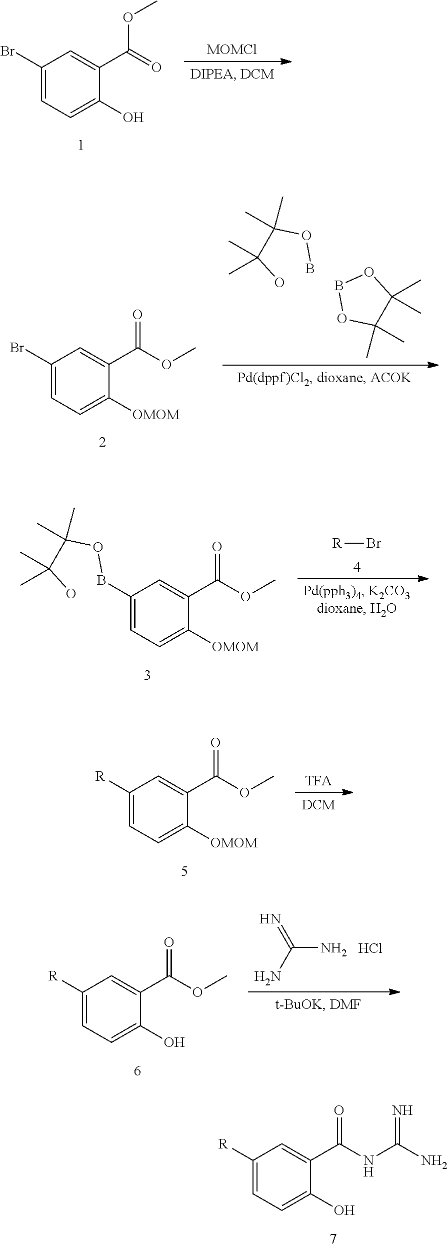

### Step 1: Synthesis of Compound 2

Hunig's base (1697 mg, 13.16 mmol) was added to a solution of compound 1 (760 mg, 3.29 mmol) in dry DCM (10 mL) at 0° C. under N₂ atmosphere followed by MOMCl (530 mg, 6.58 mmol). The resulting mixture was stirred at room temperature for 16 hours. The mixture was then poured into a saturated aq. NaHCO₃ solution and extracted with DCM twice. The combined organic layers were washed with brine, dried over Na₂SO₄, filtered and concentrated under vacuum. The residue was purified by column chromatography on silica gel (Petroleum Ether:EtOAc=30:1 to 10:1) to give compound 2 (466 mg, 51.50% yield) as a yellow oil. LC/MS (ESI) m/z: 275 (M+H)⁺.

### Step 2: Synthesis of Compound 3

Potassium acetate (107 mg, 1.09 mmol) and Pd(dppf)Cl₂ (40 mg, 0.05 mmol) were added to a mixture of compound 2 (150 mg, 0.55 mmol) and 4,4,4′,4′,5,5,5′,5′-octamethyl-2,2′-bi(1,3,2-dioxaborolane) (208 mg, 0.82 mmol) in dioxane (6 mL). The resulting mixture was stirred at 110° C. for 1.5 hours under N₂ atmosphere. The mixture was then diluted with H₂O and extracted with EtOAc twice. The combined organic layers were washed with brine, dried over anhydrous Na₂SO₄, filtered and concentrated under vacuum. The residue was purified by column chromatography on silica gel (Petroleum Ether:EtOAc=20:1 to 5:1) to give compound 3 (162 mg, 92.22% yield) as a yellow oil. LC/MS (ESI) m/z: 323 (M+H)+.

### Step 3: Synthesis of Compound 5

Potassium carbonate (2.0 eq) and Pd(PPh₃)₄ (0.1 eq) were added to a mixture of compound 3 (1.0 eq) and the appropriate pyridine bromide 4 (1.2 eq) in dioxane/H₂O (8:1). The mixture was stirred at 95° C. for 16 hours under N₂ atmosphere. The mixture was then diluted with H₂O and extracted with EtOAc twice. The combined organic layers were washed with brine, dried over anhydrous Na₂SO₄, filtered and concentrated under vacuum. The residue was purified by column chromatography on silica gel (Petroleum Ether:EtOAc=10:1 to 2:1) to give compound 5.

### Step 4: Synthesis of Compound 6

TFA (1:1, v/v) was added dropwise at 0° C. to a solution of compound 5 (1.0 eq) in DCM and the mixture was stirred at room temperature for 1 hour. The mixture was then concentrated, diluted with saturated aq. NaHCO₃ and extracted with EtOAc twice. The combined organic layers were washed with brine, dried over anhydrous Na₂SO₄, filtered and concentrated to give compound 6 without any further purification.

### Step 5: Synthesis of Compound 7

Potassium tert-butoxide (15.0 eq) was added to a solution of guanidine hydrochloride (12.0 eq) in DMF, and the mixture was stirred for 45 minutes at room temperature. Then a solution of compound 6 (1.0 eq) in DMF was added to the above solution and the resulting mixture was stirred at room temperature for 16 hours. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was triturated with MeOH and filtered to give compound 7.

### Synthesis of A57

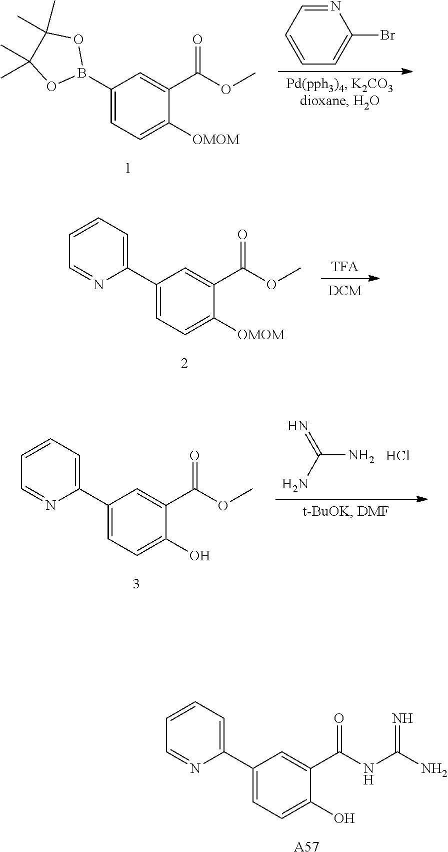

### Step 1: Synthesis of Compound 2

Potassium carbonate (146 mg, 1.06 mmol) was added to a mixture of compound 1 (170 mg, 0.53 mmol) and 2-bromopyridine (100 mg, 0.63 mmol) in dioxane (8 mL) and H₂O (1 mL) followed by Pd(pph₃)₄ (61 mg, 0.05 mmol). The resulting mixture was then stirred at 95° C. for 16 hours under N₂ atmosphere. The mixture was then diluted with H₂O and extracted with EtOAc twice. The combined organic layers were washed with brine, dried over anhydrous Na₂SO₄, filtered and concentrated under vacuum. The residue was purified by column chromatography on silica gel (Petroleum Ether:EtOAc=10:1 to 3:1) to give compound 2 (128 mg, 88.76% yield) as a colorless oil. LC/MS (ESI) m/z: 274 (M+H)⁺.

### Step 2: Synthesis of Compound 3

TFA (3 ml) was added dropwise at 0° C. to a solution of compound 2 (128 mg, 0.47 mmol) in DCM (3 mL) and the mixture was stirred at room temperature for 1 hour. The mixture was then concentrated, diluted with saturated aq. NaHCO₃ and extracted with EtOAc twice. The combined organic layers were washed with brine, dried over anhydrous Na₂SO₄, filtered and concentrated under vacuum to give compound 3 (107 mg, 99.65% yield) as a yellow solid. LC/MS (ESI) m/z: 230 (M+H)⁺.

### Step 3: Synthesis of Compound A57

Potassium tert-butoxide (785 mg, 7.01 mmol) was added to a solution of guanidine hydrochloride (535 mg, 5.60 mmol) in DMF (8 mL) and the resulting mixture was stirred at room temperature for 45 minutes. Then a solution of compound 3 (107 mg, 0.47 mmol) in DMF (2 mL) was added to the above reaction and the resulting mixture was stirred at room temperature for 16 hours. The reaction mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was triturated with MeOH and filtered to give compound A57 as a yellow solid (26.6 mg, 22.24% yield). LC/MS (ESI) m/z: 257 (M+H)⁺. ¹H NMR (400 MHz, DMSO) δ 15.21 (s, 1H), 8.60 (d, J=2.5 Hz, 2H), 8.40 (s, 1.5H), 8.04 (dd, J=8.6, 2.4 Hz, 1H), 7.85-7.79 (m, 2H), 7.28-7.23 (m, 1H), 7.09 (s, 1.5H), 6.88 (d, J=8.6 Hz, 1H).

The compounds in the table below were prepared from the appropriate starting materials described previously or commercially available using the above general procedure 4.

| Structure | Name |
|---|---|
| 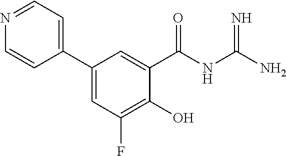 [SMILES: N=C(N)NC(=O)c1cc(-c2ccncc2)cc(F)c1O] | N-carbamimidoyl-3-fluoro-2-hydroxy-5- (pyridin-4-yl)benzamide LC-MS: m/z 275 (M + H)⁺. 1H NMR (400 MHz, DMSO) δ 8.69 (br s, 1H), 8.68 (d, J = 6.4 Hz, 2H), 8.22 (d, J = 2.2 Hz, 1H), 8.08 (d, J = 6.2 Hz, 2H), 7.93 (dd, J = 12.5, 2.4 Hz, 1H), 7.72 (br s, 1H). |
| 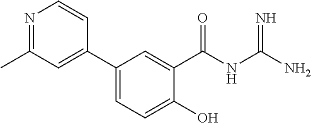 [SMILES: Cc1cc(-c2ccc(O)c(C(=O)NC(=N)N)c2)ccn1] | N-carbamimidoyl-2-hydroxy-5-(2- methylpyridin-4-yl)benzamide LC-MS: m/z 271 (M + H)⁺. 1H NMR (400 MHz, DMSO) δ 15.25 (s, 1H), 8.42 (d, J = 5.2 Hz, 1H), 8.23 (d, J = 1.6 Hz, 1H), 7.75 (dd, J = 8.5, 1.8 Hz, 1H), 7.49 (s, 1H), 7.41 (d, J = 5.1 Hz, 1H), 7.10 (br s, 2H), 6.90 (d, J = 8.5 Hz, 1H), 2.50 (s, |
| | 3H). |
| 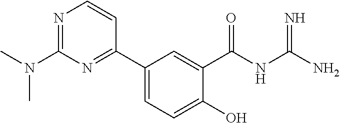 [SMILES: CN(C)c1nccc(-c2ccc(O)c(C(=O)NC(=N)N)c2)n1] | N-carbamimidoyl-5-(2- (dimethylamino)pyrimidin-4-yl)-2- hydroxybenzamide LC-MS: m/z 301 (M + H)⁺. 1H NMR (400 MHz, DMSO) δ 15.53 (s, 1H), 8.64 (d, J = 2.3 Hz, 1H), 8.44 (br s, 1H), 8.32 (d, J = 5.2 Hz, 1H), 8.09 (dd, J = 8.6, 2.3 Hz, 1H), 7.10 (br s, 1H), 7.02 (d, J = |
| | 5.2 Hz, 1H), 6.88 (d, J = 8.6 Hz, 1H), |
| | 3.19 (s, 6H). |
| 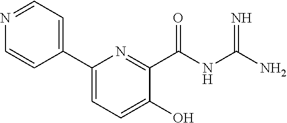 [SMILES: N=C(N)NC(=O)c1nc(-c2ccncc2)ccc1O] | N-carbamimidoyl-5-hydroxy-[2,4′- bipyridine]-6-carboxamide LC-MS: m/z 258 (M + H)⁺. 1H NMR (400 MHz, DMSO) δ 8.66 (d, J = 4.6 Hz, 1H), 8.25 (br s, 1H), 8.30-7.92 (m, 3H), 7.60 (br s, 1H), 7.43 (d, J = 7.8 Hz, 1H), 6.98 (s, 1H). |
| 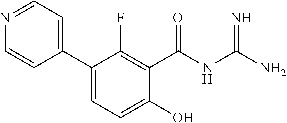 [SMILES: N=C(N)NC(=O)c1c(O)ccc(-c2ccncc2)c1F] | N-carbamimidoyl-2-fluoro-6-hydroxy-3- (pyridin-4-yl)benzamide LC-MS: m/z 275 (M + H)⁺. 1H NMR (400 MHz, DMSO) δ 8.58 (d, J = 4.8 Hz, 2H), 8.62 (br s, 2H), 7.50-7.44 (m, 3H), 7.19 (br s, 2H), 6.73 (d, J = 8.8 Hz, 1H). |
| 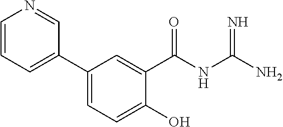 [SMILES: N=C(N)NC(=O)c1cc(-c2cccnc2)ccc1O] | N-carbamimidoyl-2-hydroxy-5-(pyridin-3- yl)benzamide LC-MS: m/z 257 (M + H)⁺. 1H NMR (400 MHz, DMSO) δ 15.10 (s, 1H), 8.82 (d, J = 2.0 Hz, 1H), 8.50 (dd, J = 4.7, 1.3 Hz, 1H), 8.42 (br s, 2H), 8.15 (d, J = 2.5 Hz, 1H), 8.03-7.95 (m, 1H), 7.69 (dd, J = 8.5, 2.5 Hz, 1H), 7.44 (dd, J = 7.9, |
| | 4.8 Hz, 1H), 7.11 (br s, 1H), 6.92 (d, J = |
| | 8.5 Hz, 1H). |
| 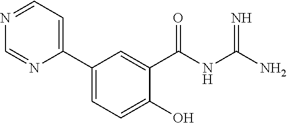 [SMILES: N=C(N)NC(=O)c1cc(-c2ccncn2)ccc1O] | N-carbamimidoyl-2-hydroxy-5- (pyrimidin-4-yl)benzamide LC-MS: m/z 258 (M + H)⁺. 1H NMR (400 MHz, DMSO) δ 15.75 (s, 1H), 9.14 (s, 1H), 8.76-8.72 (m, 2H), 8.45 (br s, 2H), 8.16 (dd, J = 8.7, 2.5 Hz, 1H), 7.95 (dd, J = 5.5, 1.3 Hz, 1H), 7.16 (br s, 1H), 6.92 (d, J = 8.7 Hz, 1H). |
| 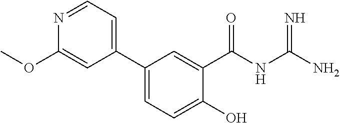 [SMILES: COc1cc(-c2ccc(O)c(C(=O)NC(=N)N)c2)ccn1] | N-carbamimidoyl-2-hydroxy-5-(2- methoxypyridin-4-yl)benzamide LC-MS: m/z 287 (M + H)⁺. 1H NMR (400 MHz, DMSO) δ 15.27 (s, 1H), 8.40 (br s, 2H), 8.20 (d, J = 2.5 Hz, 1H), 8.17 (d, J = 5.4 Hz, 1H), 7.75 (dd, J = 8.6, 2.5 Hz, 1H), 7.23 (dd, J = 5.4, 1.4 Hz, 1H), 7.10 (br s, 1H), 6.98 (s, 1H), 6.89 (d, |
| | J = 8.6 Hz, 1H), 3.88 (s, 3H). |
| 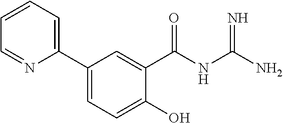 [SMILES: N=C(N)NC(=O)c1cc(-c2ccccn2)ccc1O] | N-carbamimidoyl-2-hydroxy-5-(pyridin-2- yl)benzamide LC-MS: m/z 257 (M + H)⁺. 1H NMR (400 MHz, DMSO) δ 15.21 (s, 1H), 8.60 (d, J = 2.5 Hz, 2H), 8.40 (br s, 2H), 8.04 (dd, J = 8.6, 2.4 Hz, 1H), 7.85- 7.79 (m, 2H), 7.28-7.23 (m, 1H), 7.09 (br s, 1H), 6.88 (d, J = 8.6 Hz, 1H). |
| 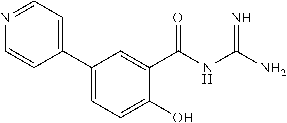 [SMILES: N=C(N)NC(=O)c1cc(-c2ccncc2)ccc1O] | N-carbamimidoyl-2-hydroxy-5-(pyridin-4- yl)benzamide LC-MS: m/z 257 (M + H)⁺. 1H NMR (400 MHz, DMSO) δ 15.31 (s, 1H), 8.56 (d, J = 6.0 Hz, 2H), 8.35 (br s, 2H), 8.25 (d, J = 2.5 Hz, 1H), 7.78 (dd, J = 8.6, 2.5 Hz, 1H), 7.62 (d, J = 6.1 Hz, 2H), 7.13 (br s, 1H), 6.92 (d, J = 8.5 Hz, 1H). |

### General Procedure 5

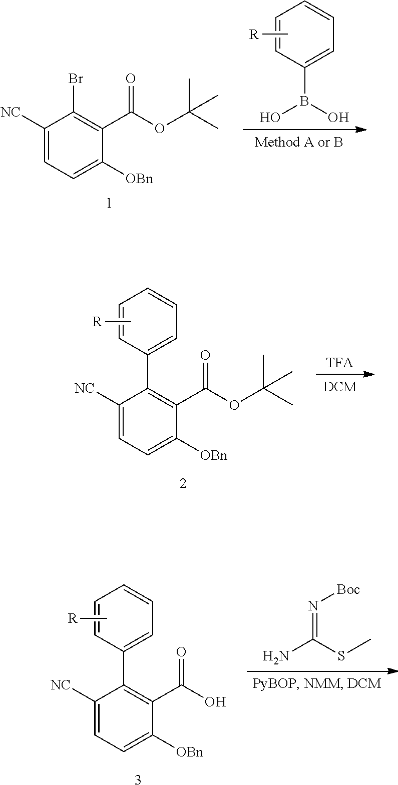

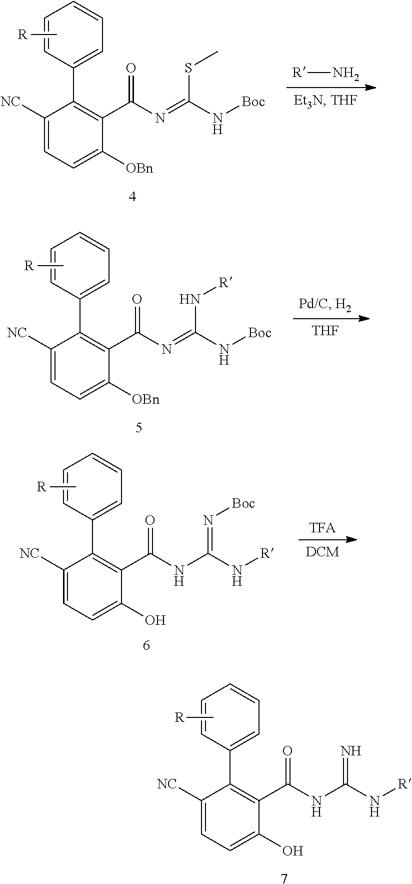

### Step 1: Synthesis of Compound 2

### Method A:

The appropriate phenylboronic acid (1.5 eq.) was added to a mixture of compound 1 (1 eq.) and K₂PO₃ (2.5 eq.) in 1,4-dioxane/H₂O (8:1) followed by Pd(OAc)₂ (0.1 eq.) and S-Phos (0.1 eq.) under N₂ atmosphere. The resulting mixture was degassed three times and stirred overnight at 95° C. under N₂ atmosphere. The mixture was then diluted with water and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=30:1 to 8:1) to give compound 2.

### Method B:

The appropriate phenylboronic acid (1.5 eq.) was added to a mixture of compound 1 (1 eq.) and potassium carbonate (2.5 eq.) in 1,4-dioxane/H₂O (8:1) followed by Pd(PPh₃)₄ (0.1 eq.) under N₂ atmosphere. The resulting mixture was degassed three times and stirred overnight at 95° C. under N₂ atmosphere. The mixture was then diluted with water and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=30:1 to 8:1) to give compound 2.

### Step 2: Synthesis of Compound 3

TFA (1:2 v/v) was added dropwise at 0° C. to a solution of compound 2 in DCM, and the reaction was stirred at room temperature for 2 hours. The reaction was then concentrated under vacuum and the residue was diluted with H₂O and extracted with DCM twice. The combined organic layers were washed with brine, dried over anhydrous Na₂SO₄, filtered and concentrated under vacuum. The residue was purified by column chromatography on silica gel (eluted with Petroleum Ether:EtOAc=10:1 to 3:1) to give compound 3.

### Step 3: Synthesis of Compound 4

tert-butyl amino(methylthio)methylenecarbamate (1.1 eq.) and PyBOP (1.5 eq.) were added to a solution of compound 3 (1.0 eq.) and NMM (5.0 eq.) in DCM and the resulting mixture was stirred at room temperature overnight under N₂ atmosphere. H₂O was added and the resulting mixture was extracted with EtOAc. The organic layer was separated, dried over anhydrous Na₂SO₄, filtered and concentrated under vacuum. The residue was purified by column chromatography on silica gel (eluted with Petroleum Ether:EA=8:1 to 2:1) to give compound 4.

### Step 4: Synthesis of Compound 5

The appropriate primary amine (1.1 eq.) was added to a solution of compound 4 (1.0 eq.) and TEA (5.0 eq.) in DCM, and the resulting mixture was stirred at room temperature for 1 hour. The mixture was then diluted with EtOAc and filtered. The filtrate was washed with brine, dried over anhydrous Na₂SO₄, filtered and concentrated under vacuum. The residue was purified by column chromatography on silica gel (eluted with Petroleum Ether:EtOAc=10:1 to 3:1) to give compound 5.

### Step 5: Synthesis of Compound 6

10% Pd/C (1:1, w/w) was added to a solution of compound 5 (1.0 eq.) in THF and the mixture was degassed under N₂ atmosphere for three times and stirred under 15 psi H₂ at room temperature for 30 minutes. The mixture was then filtered and the filtrate was concentrated under vacuum. The residue was purified by column chromatography on silica gel (eluted with Petroleum Ether:EtOAc=10:1 to 3:1) to give compound 6.

### Step 6: Synthesis of Compound 7

TFA (1:1, v/v) was added dropwise at 0° C. to a solution of compound 6 (1.0 eq.) in DCM, and the resulting solution was stirred at room temperature for 1 hour. The mixture was then concentrated under vacuum and the residue was solubilized in saturated aq. NaHCO₃ solution and extracted with EtOAc twice. The combined organic layers were washed with brine, dried over anhydrous Na₂SO₄, filtered and concentrated under vacuum. The residue was purified via prep-HPLC (C18, 0% to 50% acetonitrile in H₂O with 0.1% NH₃.H₂O) to give compound 7.

### Synthesis of A99

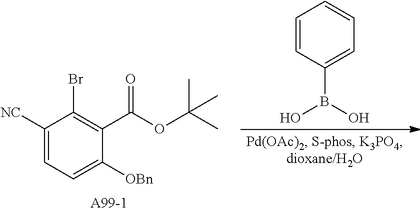

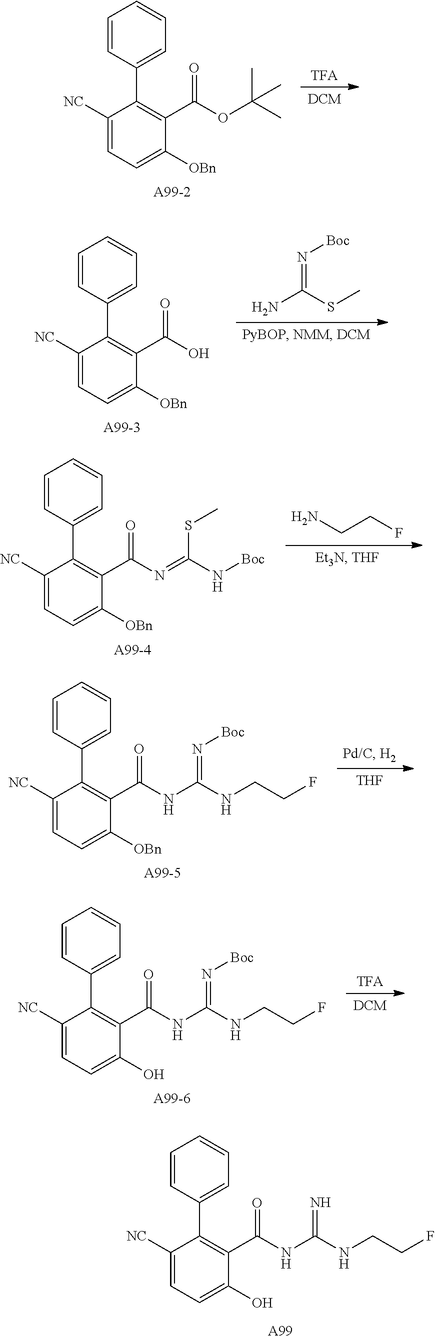

### Step 1: Synthesis of Compound A99-2

Phenylboronic acid (94 mg, 0.78 mmol) was added to a solution of compound A99-1 (200 mg, 0.52 mmol) and K₂PO₃ (273 mg, 1.31 mmol) in 1,4-dioxane (8 mL) and H₂O (1 mL) followed by Pd(OAc)₂ (12 mg, 0.05 mmol) and S-Phos (21 mg, 0.05 mmol) under N₂ atmosphere. The reaction mixture was then degassed three times and stirred overnight at 95° C. under N₂ atmosphere. The mixture was then diluted with water and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=30:1 to 8:1) to give compound A99-2 (190 mg, 0.49 mmol, 95.7% yield) as a yellow solid. LC/MS (ESI) m/z: 386 (M+H)⁺.

### Step 2: Synthesis of Compound A99-3

TFA (2 mL) was added dropwise at 0° C. to a solution of compound A-99-2 (190 mg, 0.49 mmol) in DCM (4 mL) and the reaction was stirred at room temperature for 2 hours. The mixture was then concentrated under vacuum and the residue was diluted with H₂O and extracted with DCM twice. The combined organic layers were washed with brine, dried over anhydrous Na₂SO₄, filtered and concentrated under vacuum. The residue was purified by column chromatography on silica gel (eluted with Petroleum Ether:EtOAc=10:1 to 3:1) to give compound A99-3 (155 mg, 0.47 mmol, 95.0% yield) as a yellow solid. LC/MS (ESI) m/z: 330 (M+H)⁺.

### Step 3: Synthesis of Compound A99-4

tert-butyl amino(methylthio)methylenecarbamate (99 mg, 0.52 mmol) and PyBOP (366 mg, 0.70 mmol) were added to a solution of compound A99-3 (155 mg, 0.47 mmol) and NMM (237 mg, 2.35 mmol) in DCM. The resulting mixture was stirred overnight at room temperature under N₂ atmosphere. The mixture was then washed with water and extracted with EtOAc. The organic layer was separated, dried over Na₂SO₄, filtered and concentrated under vacuum. The residue was purified by column chromatography on silica gel (eluted with Petroleum Ether:EtOAc=8:1 to 2:1) to give compound A99-4 (188 mg, 3.75 mmol, 79.6% yield) as a light yellow oil. LC/MS (ESI) m/z: 502 (M+H)⁺.

### Step 4: Synthesis of Compound A99-5

2-fluoroethanamine hydrochloride (18 mg, 0.18 mmol) was added to a solution of compound A99-4 (80 mg, 0.16 mmol) and Et₃N (81 mg, 0.80 mmol) in THF and the reaction was stirred at room temperature for 1 hour. The mixture was then diluted with EtOAc and filtered. The filtrate was washed with water and brine, dried over anhydrous Na₂SO₄, filtered and concentrated under vacuum. The residue was purified by column chromatography on silica gel (eluted with Petroleum Ether:EtOAc=10:1 to 4:1) to give compound A99-5 (60 mg, 0.17 mmol, 72.8% yield) as a light yellow solid. LC/MS (ESI) m/z: 517 (M+H)⁺.

### Step 5: Synthesis of Compound A99-6

10% Pd/C (60 mg) was added at room temperature to a solution of A99-5 (60 mg, 0.17 mmol) in THF, and the resulting mixture was degassed under N₂ atmosphere for three times and stirred under 15 psi H₂ at room temperature for 30 minutes. The mixture was then filtered and the filtrate was concentrated under vacuum. The residue was purified by column chromatography on silica gel (eluted with Petroleum Ether:EtOAc=10:1 to 3:1) to give compound A99-6 (40 mg, 0.09 mmol, 80.7% yield) as a white solid. LC/MS (ESI) m/z: 427 (M+H)⁺.

### Step 6: Synthesis of Compound A99

TFA (2 mL) was added dropwise at 0° C. to a solution of A99-6 (40 mg, 0.09 mmol) in DCM (2 mL) and the reaction was stirred at room temperature for 1 hour. The mixture was then concentrated under vacuum and the residue was solubilized in saturated aq. NaHCO₃ solution and extracted with EtOAc twice. The combined organic layers were washed with brine, dried over anhydrous Na₂SO₄, filtered and concentrated under vacuum. The residue was purified by prep-HPLC (C18, 0% to 50% acetonitrile in H₂O with 0.1% NH₃.H₂O) to give compound A99 (24 mg, 0.07 mmol, 78.4% yield) as a white solid. LC-MS: m/z 327 (M+H)⁺. ¹H NMR (400 MHz, DMSO-d₆) δ 9.14 (s, 1H), 8.56 (br s, 1H), 7.63 (d, J=9.4 Hz, 1H), 7.38-7.30 (m, 3H), 7.16 (d, J=7.0 Hz, 2H), 6.90 (d, J=8.9 Hz, 1H), 4.56-4.51 (m, 1H), 4.47-4.40 (m, 1H), 3.53-3.46 (m, 1H), 3.44-3.38 (m, 1H).

The compounds in the table below were prepared from the appropriate starting materials described previously or commercially available using the above general procedure 5.

| Structure | Name |
|---|---|
| 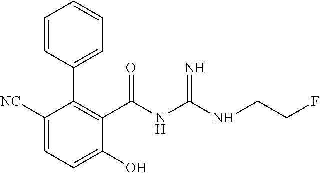 [SMILES: N#Cc1ccc(O)c(C(=O)NC(=N)NCCF)c1-c1ccccc1] | 6-cyano-N-(N-(2- fluoroethyl)carbamimidoyl)-3- hydroxy-[1,1′-biphenyl]-2- carboxamide LC-MS: m/z 327 (M + H)⁺. 1H NMR (400 MHz, DMSO) δ 9.14 (s, 1H), 8.56 (br s, 1H), 7.63 (d, J = 9.4 Hz, 1H), 7.38-7.30 (m, 3H), 7.16 (d, J = 7.0 Hz, 2H), 6.90 (d, J = 8.9 Hz, 1H), 4.56-4.51 (m, 1H), 4.47-4.40 (m, 1H), 3.53-3.46 (m, 1H), 3.44- |
| | 3.38 (m, 1H). |
| 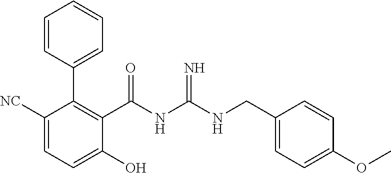 [SMILES: COc1ccc(CNC(=N)NC(=O)c2c(O)ccc(C#N)c2-c2ccccc2)cc1] | 6-cyano-3-hydroxy-N-(N-(4- methoxybenzyl)carbamimidoyl)-[1,1′- biphenyl]-2-carboxamide LC-MS: m/z 401 (M + H)⁺. 1H NMR (400 MHz, DMSO) δ 9.27 (br s, 1H), 8.47 (br s, 1H), 7.62 (d, J = 7.8 Hz, 1H), 7.36-7.23 (m, 3H), 7.22 (d, J = 6.9 Hz, 2H), 7.15 (d, J = 7.1 Hz, 2H), 6.89 (t, J = 8.7 Hz, 3H), 4.30 (s, 2H), 3.73 (s, 3H). |
| 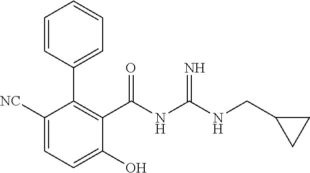 [SMILES: N#Cc1ccc(O)c(C(=O)NC(=N)NCC2CC2)c1-c1ccccc1] | 6-cyano-N-(N- (cyclopropylmethyl)carbamimidoyl)- 3-hydroxy-[1,1′-biphenyl]-2- carboxamide LC-MS: m/z 335 (M + H)⁺. 1H NMR (400 MHz, DMSO) δ 7.61 (d, J = 8.7 Hz, 1H), 7.39-7.27 (m, 3H), 7.16 (d, J = 7.6 Hz, 2H), 6.87 (d, J = 8.7 Hz, 1H), 3.02 (d, J = 6.9 Hz, 2H), 1.04-0.96 (m, 1H), 0.48-0.42 (m, 2H), 0.22-0.16 (m, 2H). |
| 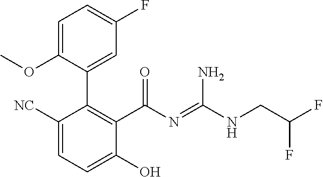 [SMILES: COc1ccc(F)cc1-c1c(C#N)ccc(O)c1C(=O)/N=C(\N)NCC(F)F] | N-(amino((2,2- difluoroethyl)amino)methylene)-6- cyano-5′-fluoro-3-hydroxy-2′- methoxy-[1,1′-biphenyl]-2- carboxamide LC-MS: m/z 393 (M + H)⁺. 1H NMR (400 MHz, DMSO) δ 8.71 (br s, 2H), 7.66 (d, J = 7.4 Hz, 1H), 7.11 (td, J = 8.7, 3.1 Hz, 1H), 7.01 (dd, J = 9.1, 4.6 Hz, 1H), 6.90 (dd, J = 8.8, 3.1 Hz, 2H), 6.12 (t, J = 55.3 Hz, |
| | 1H), 3.66-3.52 (m, 2H), 3.64 (s, 3H). |
| 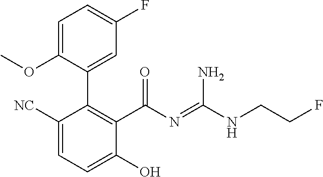 [SMILES: COc1ccc(F)cc1-c1c(C#N)ccc(O)c1C(=O)/N=C(\N)NCCF] | N-(amino((2- fluoroethyl)amino)methylene)-6- cyano-5′-fluoro-3-hydroxy-2′- methoxy-[1,1′-biphenyl]-2- carboxamide LC-MS: m/z 375 (M + H)⁺. 1H NMR (400 MHz, DMSO) δ 9.18 (br s, 1H), 8.62 (br s, 1H), 7.64 (d, J = 8.1 Hz, 1H), 7.11 (td, J = 8.7, 3.1 Hz, 1H), 7.01 (dd, J = 9.1, 4.6 Hz, 1H), 6.94-6.81 (m, 2H), 4.51 (d, J = 47.5 Hz, 2H), 3.65 (s, 3H), 3.53 (t, J = 4.8 |
| | Hz, 1H), 3.45 (dd, J = 14.8, 10.0 Hz, 1H). |
| 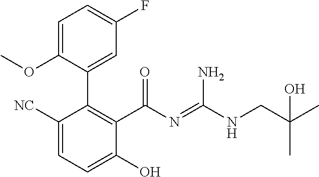 [SMILES: COc1ccc(F)cc1-c1c(C#N)ccc(O)c1C(=O)/N=C(\N)NCC(C)(C)O] | N-(amino((2-hydroxy-2- methylpropyl)amino)methylene)-6- cyano-5′-fluoro-3-hydroxy-2′- methoxy-[1,1′-biphenyl]-2- carboxamide LC-MS: m/z 401 (M + H)⁺. 1H NMR (400 MHz, DMSO) δ 9.31 (br s, 1H), 7.60 (d, J = 8.6 Hz, 1H), 7.10 (td, J = 8.7, 3.1 Hz, 1H), 7.00 (dd, J = 9.0, 4.5 Hz, 1H), 6.87 (t, J = 10.8 Hz, 1H), 4.77 (s, 1H), 3.64 (s, 3H), |
| | 3.10 (s, 2H), 1.07 (s, 6H). |
| 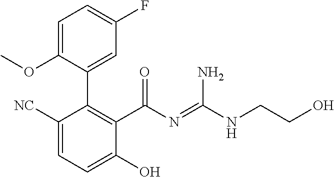 [SMILES: COc1ccc(F)cc1-c1c(C#N)ccc(O)c1C(=O)/N=C(\N)NCCO] | N-(amino((2- hydroxyethyl)amino)methylene)-6- cyano-5′-fluoro-3-hydroxy-2′- methoxy-[1,1′-biphenyl]-2- carboxamide LC-MS: m/z 373 (M + H)⁺. 1H NMR (400 MHz, DMSO) δ 9.13 (br s, 1H), 8.56 (br s, 1H), 7.60 (d, J = 8.9 Hz, 1H), 7.10 (td, J = 8.7, 3.1 Hz, 1H), 7.00 (dd, J = 9.1, 4.6 Hz, 1H), 6.85 (d, J = 7.6 Hz, 2H), 4.93 (s, |
| | 1H), 3.64 (s, 3H), 3.51-3.43 (m, 2H), |
| | 3.25-3.18 (m, 2H). |
| 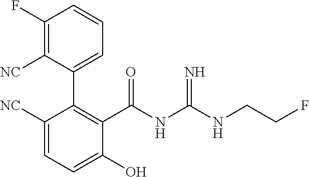 [SMILES: N#Cc1ccc(O)c(C(=O)NC(=N)NCCF)c1-c1cccc(F)c1C#N] | 2′,6-dicyano-3′-fluoro-N-(N-(2- fluoroethyl)carbamimidoyl)-3- hydroxy-[1,1′-biphenyl]-2- carboxamide LC-MS: m/z 370 (M + H)⁺. 1H NMR (400 MHz, DMSO) δ 9.18 (s, 1H), 8.61 (s, 1H), 7.85-7.75 (m, 2H), 7.52 (t, J = 8.9 Hz, 1H), 7.27 (d, J = 7.8 Hz, 1H), 7.01 (d, J = 8.7 Hz, 1H), 4.50 (d, J = 46.9 Hz, 2H), 3.52 (d, J = 27.6 Hz, 2H). |
| 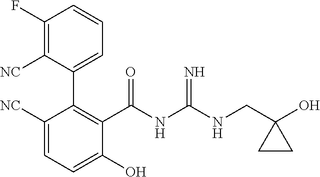 [SMILES: N#Cc1ccc(O)c(C(=O)NC(=N)NCC2(O)CC2)c1-c1cccc(F)c1C#N] | 2′,6-dicyano-3′-fluoro-3-hydroxy-N- (N-((1-hydroxycyclopropyl)methyl) carbamimidoyl)-[1,1′-biphenyl]-2- carboxamide LC-MS: m/z 394 (M + H)⁺. 1H NMR (400 MHz, DMSO) δ: 9.22 (s, 1H), 8.52 (s, 1H), 7.83-7.72 (m, 2H), 7.52 (t, J = 8.8 Hz, 1H), 7.27 (d, J = 7.7 Hz, 1H), 6.99 (d, J = 8.8 Hz, 1H), 5.59 (s, 1H), 3.28 (s, 2H), 0.66- 0.52 (m, 4H). |
| 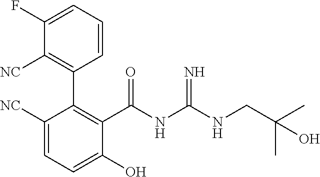 [SMILES: CC(C)(O)CNC(=N)NC(=O)c1c(O)ccc(C#N)c1-c1cccc(F)c1C#N] | 2′,6-dicyano-3′-fluoro-3-hydroxy-N- (N-(2-hydroxy-2-methylpropyl) carbamimidoyl)biphenyl-2-carboxamide LC-MS: m/z 396 (M + H)⁺. 1H NMR (400 MHz, DMSO) δ 9.27 (s, 1H), 8.49 (s, 1H), 7.84-7.71 (m, 2H), 7.52 (t, J = 8.8 Hz, 1H), 7.27 (d, J = 7.6 Hz, 1H), 7.11-6.89 (m, 1H), 4.80 (s, 1H), 3.18-3.09 (m, 2H), 1.12 (s, 6H). |
| 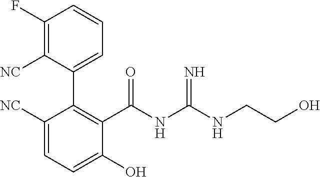 [SMILES: N#Cc1ccc(O)c(C(=O)NC(=N)NCCO)c1-c1cccc(F)c1C#N] | 2′,6-dicyano-3′-fluoro-3-hydroxy-N- (N-(2-hydroxyethyl)carbamimidoyl)- [1,1′-biphenyl]-2-carboxamide LC-MS: m/z 368 (M + H)⁺. 1H NMR (400 MHz, DMSO) δ 7.82- 7.71 (m, 2H), 7.51 (t, J = 9.0 Hz, 1H), 7.25 (d, J = 7.7 Hz, 1H), 6.96 (d, J = 8.8 Hz, 1H), 3.51 (t, J = 4.6 Hz, 2H), 3.24 (t, J = 5.0 Hz, 2H). |
| 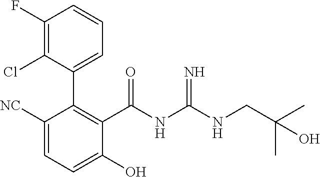 [SMILES: CC(C)(O)CNC(=N)NC(=O)c1c(O)ccc(C#N)c1-c1cccc(F)c1Cl] | 2′-chloro-6-cyano-3′-fluoro-3- hydroxy-N-(N-(2-hydroxy-2- methylpropyl)carbamimidoyl)-[1,1′- biphenyl]-2-carboxamide LC-MS: m/z 405 (M + H)⁺. 1H NMR (400 MHz, DMSO) δ 9.27 (s, 1H), 7.68 (d, J = 8.8 Hz, 1H), 7.37 (m, 2H), 7.07 (d, J = 6.2 Hz, 1H), 6.92 (d, J = 8.5 Hz, 1H), 4.79 (s, 1H), 3.11 (s, 2H), 1.11 (s, 6H). |
| 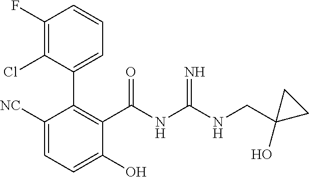 [SMILES: N#Cc1ccc(O)c(C(=O)NC(=N)NCC2(O)CC2)c1-c1cccc(F)c1Cl] | 2′-chloro-6-cyano-3′-fluoro-3- hydroxy-N-(N-((1- hydroxycyclopropyl)methyl) carbamimidoyl)- [1,1′-biphenyl]-2-carboxamide LC-MS: m/z 403 (M + H)⁺. 1H NMR (400 MHz, DMSO) δ 9.22 (s, 1H), 7.69 (d, J = 8.7 Hz, 1H), 7.41- 7.34 (m, 2H), 7.11-7.02 (m, 1H), 6.93 (d, J = 9.0 Hz, 1H), 5.59 (s, 1H), 3.27 (s, 2H), 0.64-0.51 (m, 4H). |
| 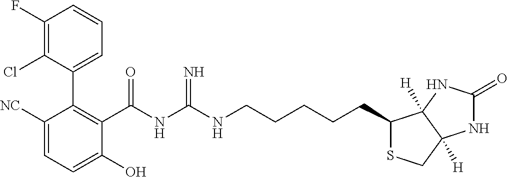 [SMILES: N#Cc1ccc(O)c(C(=O)NC(=N)NCCCCC[C@@H]2SC[C@@H]3NC(=O)N[C@@H]32)c1-c1cccc(F)c1Cl] | 2′-chloro-6-cyano-3′-fluoro-3- hydroxy-N-(N-(5-((3aS,4S,6aR)-2- oxohexahydro-1H-thieno[3,4- d]imidazol-4- yl)pentyl)carbamimidoyl)-[1,1′- biphenyl]-2-carboxamide LC-MS: m/z 545 (M + H)⁺. 1H NMR (400 MHz, DMSO) δ 8.90 (s, 1H), 8.09 (br s, 1H), 7.68 (d, J = 8.3 Hz, 1H), 7.41-7.32 (m, 2H), 7.09- 7.02 (m, 1H), 6.94 (d, J = 8.9 Hz, 1H), 6.30 (s, 1H), 6.26 (s, 1H), 4.36-4.28 |
| | (m, 1H), 4.19-4.12 (m, 1H), 3.19- |
| | 3.09 (m, 3H), 2.83 (dd, J = 12.4, 5.1 |
| | Hz, 1H), 2.60 (d, J = 12.4 Hz, 1H), |
| | 1.65-1.28 (m, 8H). |
| 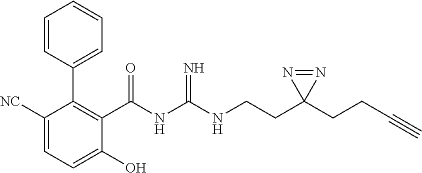 [SMILES: C#CCCC1(CCNC(=N)NC(=O)c2c(O)ccc(C#N)c2-c2ccccc2)N=N1] | N-(N-(2-(3-(but-3-yn-1-yl)-3H- diazirin-3-yl)ethyl)carbamimidoyl)-6- cyano-3-hydroxy-[1,1′-biphenyl]-2- carboxamide LC-MS: m/z 401 (M + H)⁺. 1H NMR (400 MHz, DMSO) δ 8.88 (s, 1H), 8.42 (br s, 1H), 7.62 (d, J = 8.3 Hz, 1H), 7.42-7.27 (m, 3H), 7.16 (d, J = 6.5 Hz, 2H), 6.88 (d, J = 8.4 Hz, 1H), 3.08-2.94 (m, 2H), 2.81 (s, 1H), 2.07-1.90 (m, 2H), 1.71-1.52 (m, 4H). |
| 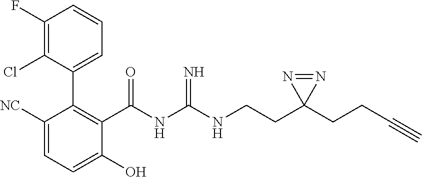 [SMILES: C#CCCC1(CCNC(=N)NC(=O)c2c(O)ccc(C#N)c2-c2cccc(F)c2Cl)N=N1] | N-(N-(2-(3-(but-3-yn-1-yl)-3H- diazirin-3-yl)ethyl)carbamimidoyl)-2′- chloro-6-cyano-3′-fluoro-3-hydroxy- [1,1′-biphenyl]-2-carboxamide LC-MS: m/z 453 (M + H)⁺. 1H NMR (400 MHz, DMSO) δ 8.53 (br s, 1H), 7.69 (d, J = 8.9 Hz, 1H), 7.42-7.32 (m, 2H), 7.06 (d, J = 5.8 Hz, 1H), 6.92 (d, J = 8.6 Hz, 1H), 3.11- 2.96 (m, 2H), 2.81 (s, 1H), 2.04- 1.90 (m, 2H), 1.75-1.48 (m, 4H). |
| 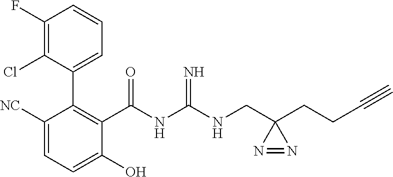 [SMILES: C#CCCC1(CNC(=N)NC(=O)c2c(O)ccc(C#N)c2-c2cccc(F)c2Cl)N=N1] | N-(N-((3-(but-3-yn-1-yl)-3H-diazirin- 3-yl)methyl)carbamimidoyl)-2′-chloro- 6-cyano-3′-fluoro-3-hydroxy-[1,1′- biphenyl]-2-carboxamide LC-MS: m/z 439 (M + H)⁺. 1H NMR (400 MHz, DMSO) δ 8.67 (br, 1H), 7.73 (s, 1H), 7.43-7.33 (m, 2H), 7.01 (m, 2H), 3.30 (m, 2H), 2.81 (s, 1H), 1.99 (m, 2H), 1.66 (m, 2H). |
| 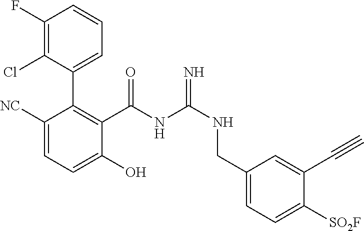 [SMILES: C#Cc1cc(CNC(=N)NC(=O)c2c(O)ccc(C#N)c2-c2cccc(F)c2Cl)ccc1S(=O)(=O)F] | 4-((3-(2′-chloro-6-cyano-3′-fluoro-3- hydroxy-[1,1′-biphenyl]-2- carbonyl)guanidino)methyl)-2- ethynylbenzenesulfonyl fluoride LC-MS: m/z 529 (M + H)⁺. 1H NMR (400 MHz, DMSO) δ 9.45 (s, 1H), 8.18 (d, J = 8.3 Hz, 1H), 7.78 (d, J = 1.2 Hz, 1H), 7.74 (d, J = 8.7 Hz, 1H), 7.62 (d, J = 4.9 Hz, 1H), 7.40- 7.35 (m, 2H), 7.08 (d, J = 5.0 Hz, 1H), 6.97 (d, J = 8.2 Hz, 1H), 4.98 (s, 1H), 4.57 (d, J = 6.3 Hz, 2H). |
| 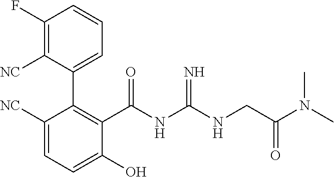 [SMILES: CN(C)C(=O)CNC(=N)NC(=O)c1c(O)ccc(C#N)c1-c1cccc(F)c1C#N] | 2′,6-dicyano-N-(N-(2- (dimethylamino)-2- oxoethyl)carbamimidoyl)-3′-fluoro-3- hydroxy-[1,1′-biphenyl]-2- carboxamide LC-MS: m/z 409 (M + H)⁺. 1H NMR (400 MHz, DMSO) δ 9.43 (s, 1H), 7.87 (s, 1H), 7.82-7.70 (m, 2H), 7.50 (dd, J = 8.8, 8.8 Hz, 1H), 7.25 (d, J = 7.2 Hz, 1H), 7.08-6.94 (m, 1H), 4.09 (s, 2H), 2.92 (s, 3H), 2.86 (s, 3H). |

### General Procedure 6

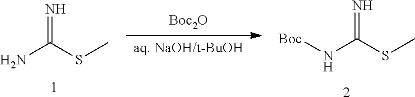

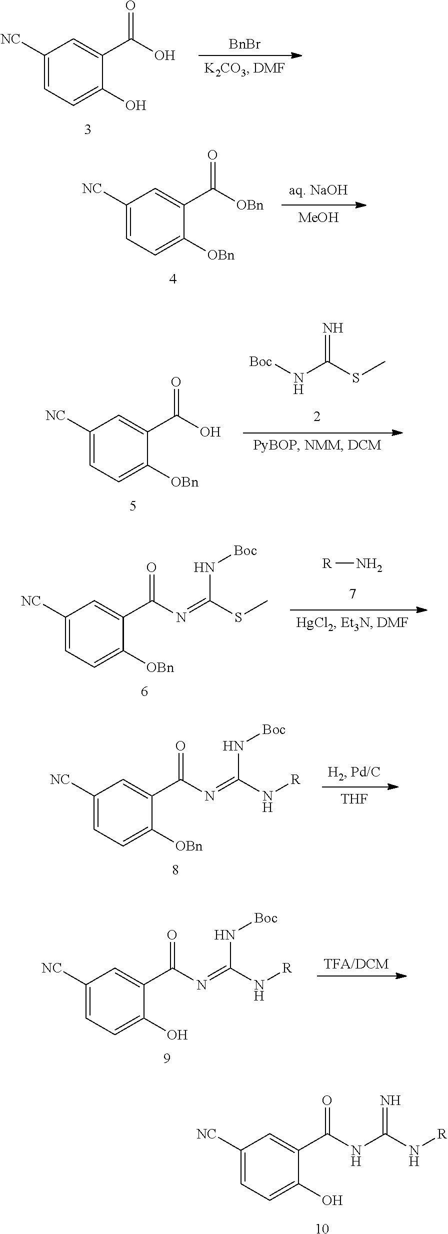

### Step 1: Synthesis of Compound 2

Methyl carbamimidothioate (9 g, 100 mmol) was dissolved in a solution of NaOH (4 g, 100 mmol) in water (10 mL) and t-BuOH (100 mL). A solution of Boc₂O (19.6 g, 90 mmol) in t-BuOH (50 mL) was then added dropwise at 0° C. over a period of 1 hour and the resulting mixture was stirred overnight at room temperature. The mixture was then diluted with water and extracted with DCM. The organic layer was washed with brine, dried over anhydrous Na₂SO₄, filtered and concentrated under vacuum. The residue was purified by column chromatography on silica gel (eluted with Petroleum Ether:EtOAc=100:0 to 5:1) to give compound 2 (8.1 g, 42.6% yield) as a white solid. LC/MS (ESI) m/z: 191 (M+H)⁺.

### Step 2: Synthesis of Compound 4

Benzyl bromide (8.55 g, 50 mmol) was added dropwise to a mixture of compound 3 (3.26 g, 20 mmol) and potassium carbonate (8.28 g, 60 mmol) in DMF (40 mL) and the resulting mixture was stirred at room temperature for 16 hours. After the reaction was completed, the mixture was diluted with EtOAc and washed with water. The organic layer was then dried over Na₂SO₄, filtered and concentrated under vacuum. The residue was purified by column chromatography on silica gel (eluted with Petroleum Ether:EtOAc=100:0 to 20:1) to give compound 4 (3.3 g, 48.1% yield) as a colorless oil. LC/MS (ESI) m/z: 366 (M+Na)⁺.

### Step 3: Synthesis of Compound 5

A solution of NaOH (962 mg, 24 mmol) in water (10 mL) was added to a solution of compound 4 (3.3 g, 9.62 mmol) in MeOH (20 mL) and the resulting mixture was stirred at room temperature for 16 hours. The mixture was then diluted with water and extracted with Et₂O. The aqueous layer was separated and acidified with 1 N HCl solution to pH=5. Then the mixture was extracted with EtOAc twice and the combined organic layers were dried over anhydrous Na₂SO₄, filtered and concentrated under vacuum. The residue was purified by column chromatography on silica gel (eluted with DCM:MeOH=100:0 to 40:1) to give compound 5 (2.1 g, 86.3% yield) as a colorless oil. LC/MS (ESI) m/z: 252 (M−H)⁻.

The synthesis from 6 to 10 is the same as in the general procedure 5 described above.

### Synthesis of F28

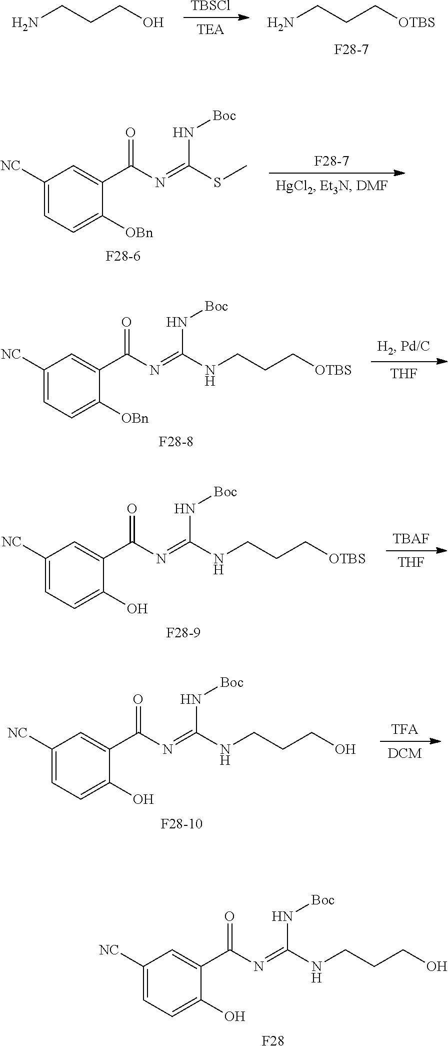

### Step 1: Synthesis of Compound F28-7

TBSCl (1.1 g, 7.3 mmol) was added at 0° C. to a solution of 1-amino-3-propanol (0.5 g, 6.6 mmol) and Et₃N (1.4 mL, 9.9 mmol) in DCM (15 mL), and the resulting mixture was stirred at room temperature for 12 hours. The mixture was then washed with water and the organic layer was separated, dried over MgSO₄, filtered and concentrated under vacuum. The residue was purified by column chromatography on silica gel (eluted with DCM:MeOH=20:1 to 5:1) to give compound F28-7 as a yellow oil (1.2 g, 6.3 mmol, 95% yield).

The synthesis from F28-6 to F28 is the same as in the general procedure 5 described above.

The compounds in the table below were prepared from the appropriate starting materials described previously or commercially available using the above general procedure 6.

| Structure | Name |
|---|---|
| 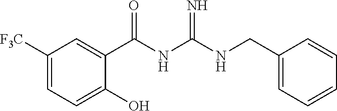 [SMILES: N=C(NCc1ccccc1)NC(=O)c1cc(C(F)(F)F)ccc1O] | N-(N-benzylcarbamimidoyl)-2- hydroxy-5-(trifluoromethyl)benzamide LC-MS: m/z 338 (M + H)⁺. 1H NMR (400 MHz, DMSO-d6) δ 15.49 (s, 1H), 9.67 (s, 1H), 8.82 (s, 1H), 8.11 (s, 1H), 7.82 (s, 1H), 7.61 (d, J = 8.5 Hz, 1H), 7.45-7.21 (m, 5H), 6.95 (d, J = 8.6 Hz, 1H), 4.47 (s, 2H). |
| 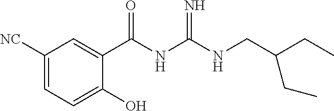 [SMILES: CCC(CC)CNC(=N)NC(=O)c1cc(C#N)ccc1O] | 5-cyano-N-(N-(2- ethylbutyl)carbamimidoyl)-2- hydroxybenzamide LC-MS m/z 289 (M + H)⁺. 1H NMR (400 MHz, DMSO-d6) δ 16.17 (s, 1H), 9.46 (s, 1H), 8.60 (s, 1H), 8.12 (s, 1H), 7.66 (d, J = 8.7 Hz, 1H), 6.87 (d, J = 8.6 Hz, 1H), 3.15 (d, J = 8.0 |
| | Hz, 2H), 1.57-1.48 (m, 1H), 1.35 (dd, |
| | J = 14.5, 7.1 Hz, 4H), 0.87 (t, J = 7.4 Hz, 7H). |
| 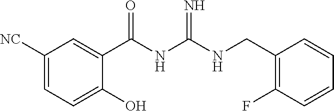 [SMILES: N#Cc1ccc(O)c(C(=O)NC(=N)NCc2ccccc2F)c1] | 5-cyano-N-(N-(2- fluorobenzyl)carbamimidoyl)-2- hydroxybenzamide LC-MS: m/z 313 (M + H)⁺. 1H NMR (400 MHz, DMSO-d6) δ 15.96 (s, 1H), 9.65 (s, 1H), 8.90 (br s, 1H), 8.13 (s, 1H), 7.68 (d, J = 7.9 Hz, 1H), 7.47-7.33 (m, 2H), 7.30-7.18 (m, 2H), 6.90 (d, J = 8.6 Hz, 1H), 4.53 (s, 2H). |
| 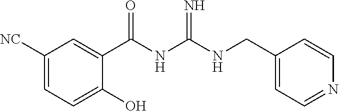 [SMILES: N#Cc1ccc(O)c(C(=O)NC(=N)NCc2ccncc2)c1] | 5-cyano-2-hydroxy-N-(N-(pyridin-4- ylmethyl)carbamimidoyl)benzamide LC-MS: m/z 296 (M + H)⁺. 1H NMR (400 MHz, DMSO-d6) δ 16.00 (s, 1H), 8.56 (d, J = 5.2 Hz, 2H), 8.14 (d, J = 2.0 Hz, 1H), 7.69 (dd, J = 8.6, 2.3 Hz, 1H), 7.32 (d, J = 5.8 Hz, 2H), 6.90 (d, J = 8.3 Hz, 1H), 4.53 (s, 2H). |
| 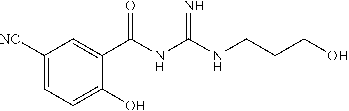 [SMILES: N#Cc1ccc(O)c(C(=O)NC(=N)NCCCO)c1] | 5-cyano-2-hydroxy-N-(N-(3- hydroxypropyl)carbamimidoyl)benzamide LC-MS: m/z 263 (M + H)⁺. 1H NMR (400 MHz, DMSO-d6) δ 16.22 (s, 1H), 9.29 (s, 1H), 8.11 (s, 1H), 7.65 (d, J = 8.4 Hz, 1H), 6.86 (d, J = 8.6 Hz, 1H), 4.64 (d, J = 5.2 Hz, 1H), 3.52- |
| | 3.46 (m, 3H), 3.29-3.23 (m, 3H), |
| | 1.74-1.66 (m, 2H). |
| 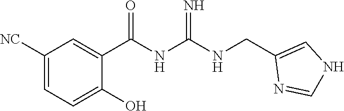 [SMILES: N#Cc1ccc(O)c(C(=O)NC(=N)NCc2c[nH]cn2)c1] | N-(N-((1H-imidazol-4- yl)methyl)carbamimidoyl)-5-cyano-2- hydroxybenzamide LC-MS: m/z 285 (M + H)⁺. 1H NMR (400 MHz, DMSO-d6) δ 16.07 (s, 1H), 9.15 (br s, 2H), 8.10 (d, J = 1.7 Hz, 1H), 7.69-7.61 (m, 2H), |
| | 7.09 (s, 1H), 6.86 (d, J = 8.6 Hz, 1H), |
| | 4.32 (s, 2H). |
| 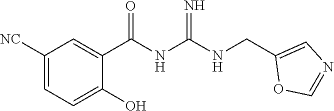 [SMILES: N#Cc1ccc(O)c(C(=O)NC(=N)NCc2cnco2)c1] | 5-cyano-2-hydroxy-N-(N-(oxazol-5- ylmethyl)carbamimidoyl)benzamide LC-MS: m/z 286 (M + H)⁺. 1H NMR (400 MHz, DMSO-d6) δ 15.81 (s, 1H), 8.66 (br s, 2H), 8.37 (s, 1H), 8.14 (s, 1H), 7.68 (d, J = 8.2 Hz, 1H), 7.14 (s, 1H), 6.90 (d, J = 8.5 Hz, 1H), 4.56 (s, 2H). |

### General Procedure 7

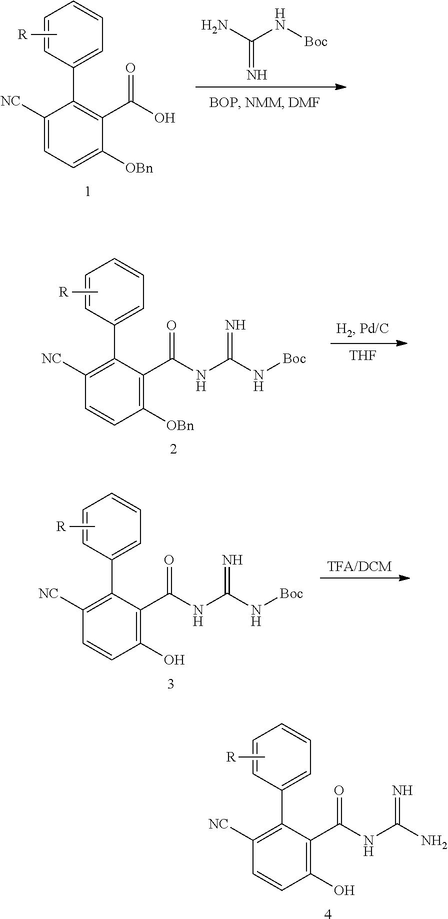

### Step 1: Synthesis of Compound 1

The synthesis of compound 1 was described in general procedure 5.

### Step 2: Synthesis of Compound 2

tert-butyl N-carbamimidoylcarbamate (2.6 eq.) and PyBOP (1.4 eq.) were added under N₂ atmosphere to a mixture of 1 (1 eq.) and NMM (4 eq.) in DMF. The resulting mixture was stirred at room temperature for 15 hours. The mixture was then poured into water and extracted with EtOAc twice. The combined organic layers were washed with brine, dried over Na₂SO₄, filtered and concentrated under vacuum. The residue was purified by flash column chromatography on silica gel (eluted with Petroleum Ether:EA=10:1 to 3:1) to give compound 2.

### Step 4: Synthesis of Compound 3

10% Pd/C (1:1, w/w) was added under N₂ atmosphere to a solution of 2 (1 eq.) in THF and the resulting mixture was stirred at room temperature under H₂ atmosphere for 1 hour. The mixture was then filtered and the filtrate was concentrated under vacuum to give compound 3.

### Step 5: Synthesis of Compound 4

TFA (1:1, v/v) was added dropwise at 0° C. to a solution of 3 (1 eq.) in DCM and the reaction mixture was slowly warmed to room temperature and stirred for 2 hours. The solvent was then removed under vacuum and the crude was purified by prep-HPLC (C18, 0% to 50% acetonitrile in H₂O with 0.1% NH₃.H₂O) to give compound 4.

### Synthesis of A39

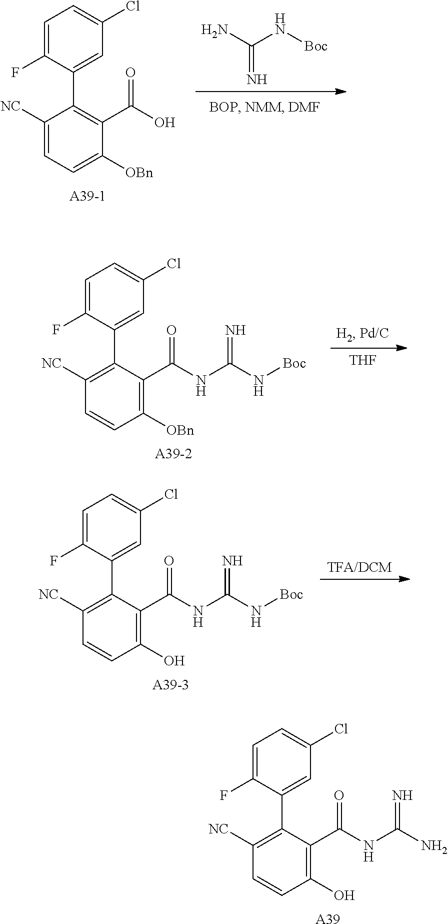

### Step 1: Synthesis of Compound A39-2

tert-butyl N-carbamimidoylcarbamate (91.0 mg, 0.572 mmol) was added under N₂ atmosphere to a solution of A39-1 (84 mg, 0.22 mmol) and NMM (88.9 mg, 0.88 mmol) in DMF (6 mL) followed by PyBOP (136.2 mg, 0.31 mmol). The resulting mixture was stirred at room temperature for 15 hours. The mixture was then poured into water and extracted with EtOAc twice. The combined organic layers were washed with brine, dried over Na₂SO₄, filtered and concentrated under vacuum. The residue was purified by flash column chromatography on silica gel (eluted with Petroleum Ether:EA=10:1 to 3:1) to give A39-2 (100 mg, 86.92% yield) as a white solid. LC/MS (ESI) m/z: 523 (M+H)⁺.

### Step 2: Synthesis of Compound A39-3

10% Pd/C (100 mg) was added under N₂ atmosphere to a solution of A39-2 (100 mg, 0.19 mmol) in THF (5 mL) and the mixture was stirred at room temperature under 15 psi H₂ for 1 hour. The mixture was then filtered and the filtrate was concentrated to give A39-2 (80 mg, 96.7% yield) as a white solid without any further purification. LC/MS (ESI) m/z: 433 (M+H)⁺.

### Step 3: Synthesis of Compound A39

TFA (2 mL) was added at 0° C. dropwise to a solution of A39-3 (80 mg, 0.185 mmol) in DCM (2 mL) and the mixture was slowly warmed to room temperature and stirred for 1 hour. The solvent was then removed under vacuum and the residue was purified via prep-HPLC (C18, 0% to 50% acetonitrile in H₂O with 0.1% NH₃.H₂O) to give A39 (18 mg, 29.27% yield) as a white solid. LC/MS (ESI) m/z: 333 (M+H)⁺, 1H NMR (400 MHz, DMSO) δ: 7.66 (d, J=8.8 Hz, 1H), 7.43 (ddd, J=8.7, 4.4, 2.7 Hz, 1H), 7.32 (dd, J=6.3, 2.7 Hz, 1H), 7.26 (t, J=9.0 Hz, 1H), 6.90 (d, J=8.8 Hz, 1H).

The compounds in the table below were prepared from the appropriate starting materials described previously or commercially available using the above general procedure 7

| Structure | Name |
|---|---|
| 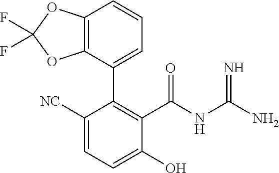 [SMILES: N#Cc1ccc(O)c(C(=O)NC(=N)N)c1-c1cccc2c1OC(F)(F)O2] | N-carbamimidoyl-3-cyano-2-(2,2- difluorobenzo[d][1,3]dioxol-4-yl)-6- hydroxybenzamide LC-MS: m/z 361 (M + H)⁺. 1H NMR (400 MHz, DMSO) δ 7.67 (d, J = 8.8 Hz, 1H), 7.35 (dd, J = 8.1, 1.0 Hz, 1H), 7.20 (t, J = 8.0 Hz, 1H), 7.04 (dd, J = 8.0, 1.0 Hz, 1H), 6.91 (d, J = 8.8 Hz, 1H). |
| 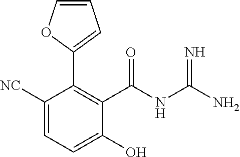 [SMILES: N#Cc1ccc(O)c(C(=O)NC(=N)N)c1-c1ccco1] | N-carbamimidoyl-3-cyano-2-(furan-2-yl)-6- hydroxybenzamide LC-MS: m/z 271 (M + H)⁺. 1H NMR (400 MHz, DMSO) δ 7.68 (s, 1H), 7.61 (d, J = 8.8 Hz, 1H), 6.87 (d, J = 8.7 Hz, 1H), 6.55-6.50 (m, 1H), 6.47 (d, J = 3.2 Hz, 1H). |
| 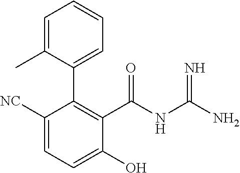 [SMILES: Cc1ccccc1-c1c(C#N)ccc(O)c1C(=O)NC(=N)N] | N-carbamimidoyl-6-cyano-3-hydroxy-2′-methyl- [1,1′-biphenyl]-2-carboxamide LC-MS: m/z 295 (M + H)⁺. 1H NMR (400 MHz, DMSO) δ 8.25 (br s, 2H), 7.62 (d, J = 8.7 Hz, 1H), 7.20-7.13 (m, 3H), 6.93 (d, J = 7.2 Hz, 1H), 6.85 (d, J = 8.7 Hz, 1H), 1.97 (s, 3H). |
| 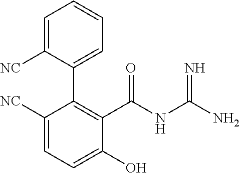 [SMILES: N#Cc1ccccc1-c1c(C#N)ccc(O)c1C(=O)NC(=N)N] | N-carbamimidoyl-2′,6-dicyano-3-hydroxy-[1,1′- biphenyl]-2-carboxamide LC-MS: m/z 306 (M + H)⁺. 1H NMR (400 MHz, DMSO) δ 7.85 (d, J = 7.6 Hz, 1H), 7.81 (br s, 2H), 7.78-7.62 (m, 2H), 7.51 (t, J = 8.0 Hz, 1H), 7.36 (d, J = 7.6 Hz, 1H), 6.91 (d, J = 8.8 Hz, 1H). |
| 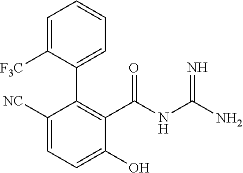 [SMILES: N#Cc1ccc(O)c(C(=O)NC(=N)N)c1-c1ccccc1C(F)(F)F] | N-carbamimidoyl-6-cyano-3-hydroxy-2′- (trifluoromethyl)-[1,1′-biphenyl]-2-carboxamide LC-MS: m/z 349 (M + H)⁺. 1H NMR (400 MHz, DMSO) δ 8.24 (br s, 2H), 7.73 (d, J = 8.0 Hz, 1H), 7.68-7.58 (m, 2H), 7.53 (t, J = 7.6 Hz, 1H), 7.24 (d, J = 7.6 Hz, 1H), 7.22 (br s, 1H), 6.88 (d, J = 8.8 Hz, 1H). |
| 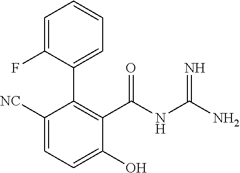 [SMILES: N#Cc1ccc(O)c(C(=O)NC(=N)N)c1-c1ccccc1F] | N-carbamimidoyl-6-cyano-2′-fluoro-3-hydroxy- [1,1′-biphenyl]-2-carboxamide LC-MS: m/z 299 (M + H)⁺. 1H NMR (400 MHz, DMSO) δ 7.74 (br s, 2H), 7.63 (d, J = 8.8 Hz, 1H), 7.41-7.32 (m, 1H), 7.25-7.12 (m, 3H), 6.87 (d, J = 8.4 Hz, 1H). |
| 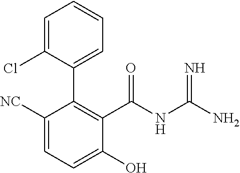 [SMILES: N#Cc1ccc(O)c(C(=O)NC(=N)N)c1-c1ccccc1Cl] | N-carbamimidoyl-2′-chloro-6-cyano-3-hydroxy- [1,1′-biphenyl]-2-carboxamide LC-MS: m/z 315 (M + H)⁺. NMR (400 MHz, DMSO) δ 7.74 (br s, 2H), 7.63 (d, J = 8.7 Hz, 1H), 7.47-7.40 (m, 1H), 7.33 (dd, J = 5.7, 3.4 Hz, 2H), 7.22-7.15 (m, 1H), 6.87 (d, J = 8.7 Hz, 1H). |
| 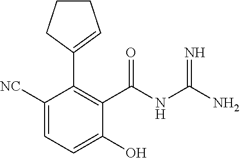 [SMILES: N#Cc1ccc(O)c(C(=O)NC(=N)N)c1C1=CCCC1] | N-carbamimidoyl-3-cyano-2-(cyclopent-1-en-1- yl)-6-hydroxybenzamide LC-MS: m/z 271 (M + H)⁺. 1H NMR (400 MHz, DMSO) δ 7.85 (br s, 3H), 7.53 (d, J = 8.8 Hz, 1H), 6.74 (d, J = 8.8 Hz, 1H), 5.44 (s, 1H), 2.60-2.52 (m, 2H), 2.48-2.38 (m, 2H), 2.05-1.93 (m, 2H). |
| 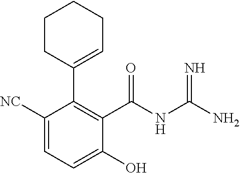 [SMILES: N#Cc1ccc(O)c(C(=O)NC(=N)N)c1C1=CCCCC1] | N-carbamimidoyl-6-cyano-3-hydroxy-2′,3′,4′,5′- tetrahydro-[1,1′-biphenyl]-2-carboxamide LC-MS: m/z 285 (M + H)⁺. 1H NMR (400 MHz, DMSO) δ 8.27 (br s, 2H), 7.51 (d, J = 8.4 Hz, 1H), 7.32 (br s, 1H), 6.72 (d, J = 8.4 Hz, 1H), 5.31 (s, 1H), 2.38-1.86 (m, 4H), 1.80-1.51 (m, 4H). |
| 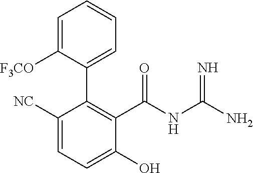 [SMILES: N#Cc1ccc(O)c(C(=O)NC(=N)N)c1-c1ccccc1OC(F)(F)F] | N-carbamimidoyl-6-cyano-3-hydroxy-2′- (trifluoromethoxy)-[1,1′-biphenyl]-2- carboxamide LC-MS: m/z 365 (M + H)⁺. 1H NMR (400 MHz, DMSO) δ 7.77 (br s, 2H), 7.65 (d, J = 8.8 Hz, 1H), 7.46 (t, J = 7.6 Hz, 1H), 7.43-7.33 (m, 2H), 7.30 (d, J = 7.6 Hz, 1H), 6.89 (d, J = 8.8 Hz, 1H). |
| 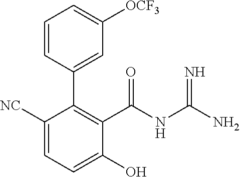 [SMILES: N#Cc1ccc(O)c(C(=O)NC(=N)N)c1-c1cccc(OC(F)(F)F)c1] | N-carbamimidoyl-6-cyano-3-hydroxy-3′- (trifluoromethoxy)-[1,1′-biphenyl]-2- carboxamide LC-MS: m/z 365 (M + H)⁺. 1H NMR (400 MHz, DMSO) δ 7.62 (d, J = 8.7 Hz, 1H), 7.47 (t, J = 7.9 Hz, 1H), 7.30 (d, J = 8.2 Hz, 1H), 7.19 (d, J = 7.6 Hz, 1H), 7.15 (s, 1H), 6.87 (d, J = 8.7 Hz, 1H). |
| 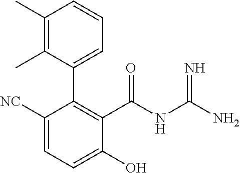 [SMILES: Cc1cccc(-c2c(C#N)ccc(O)c2C(=O)NC(=N)N)c1C] | N-carbamimidoyl-6-cyano-3-hydroxy-2′,3′- dimethyl-[1,1′-biphenyl]-2-carboxamide LC-MS: m/z 309 (M + H)⁺. 1H NMR (400 MHz, DMSO) δ 8.25 (br s, 2H), 7.60 (d, J = 8.8 Hz, 1H), 7.14 (br s, 1H), 7.09- 7.01 (m, 2H), 6.83 (d, J = 8.8 Hz, 1H), 6.76 (d, J = 7.2 Hz, 1H), 2.26 (s, 3H), 1.88 (s, 3H). |
| 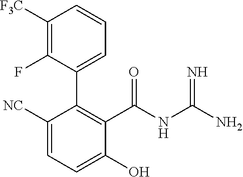 [SMILES: N#Cc1ccc(O)c(C(=O)NC(=N)N)c1-c1cccc(C(F)(F)F)c1F] | N-carbamimidoyl-6-cyano-2′-fluoro-3-hydroxy- 3′-(trifluoromethyl)-[1,1′-biphenyl]-2- carboxamide LC-MS: m/z 367 (M + H)⁺. 1H NMR (400 MHz, DMSO) δ 7.75 (t, J = 6.8 Hz, 1H), 7.69 (d, J = 8.8 Hz, 1H), 7.57 (t, J = 6.8 Hz, 1H), 7.41 (t, J = 8.0 Hz, 1H), 6.92 (d, J = 8.8 Hz, 1H). |
| 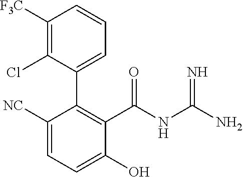 [SMILES: N#Cc1ccc(O)c(C(=O)NC(=N)N)c1-c1cccc(C(F)(F)F)c1Cl] | N-carbamimidoyl-2′-chloro-6-cyano-3-hydroxy- 3′-(trifluoromethyl)-[1,1′-biphenyl]-2- carboxamide LC-MS: m/z 383 (M + H)⁺. 1H NMR (400 MHz, DMSO) δ 7.82 (dd, J = 7.2, 1.9 Hz, 1H), 7.68 (d, J = 8.8 Hz, 1H), 7.54 (q, J = 7.7 Hz, 2H), 6.91 (d, J = 8.8 Hz, 1H). |
| 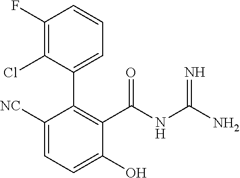 [SMILES: N#Cc1ccc(O)c(C(=O)NC(=N)N)c1-c1cccc(F)c1Cl] | N-carbamimidoyl-2′-chloro-6-cyano-3′-fluoro-3- hydroxy-[1,1′-biphenyl]-2-carboxamide LC-MS: m/z 333 (M + H)⁺. 1H NMR (400 MHz, DMSO) δ 8.26 (br s, 2H), 7.67 (d, J = 8.8 Hz, 1H), 7.43-7.28 (m, 2H), 7.22 (br s, 1H), 7.06 (d, J = 6.4 Hz, 1H), 6.90 (d, J = 8.4 Hz, 1H). |
| 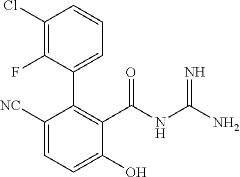 [SMILES: N#Cc1ccc(O)c(C(=O)NC(=N)N)c1-c1cccc(Cl)c1F] | N-carbamimidoyl-3′-chloro-6-cyano-2′-fluoro-3- hydroxy-[1,1′-biphenyl]-2-carboxamide LC-MS: m/z 333 (M + H)⁺. 1H NMR (400 MHz, DMSO) δ 8.08 (br s, 2H), 7.68 (d, J = 8.8 Hz, 1H), 7.55 (t, J = 7.2 Hz, 1H), 7.28-7.15 (m, 2H), 6.92 (d, J = 8.8 Hz, 1H). |
| 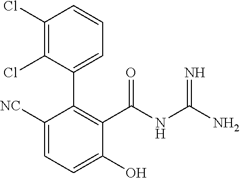 [SMILES: N#Cc1ccc(O)c(C(=O)NC(=N)N)c1-c1cccc(Cl)c1Cl] | N-carbamimidoyl-2′,3′-dichloro-6-cyano-3- hydroxy-[1,1′-biphenyl]-2-carboxamide LC-MS: m/z 349 (M + H)⁺. 1H NMR (400 MHz, DMSO) δ 7.66 (d, J = 8.7 Hz, 1H), 7.58 (d, J = 8.0 Hz, 1H), 7.36 (t, J = 7.9 Hz, 1H), 7.18 (d, J = 7.6 Hz, 1H), 6.89 (d, J = 8.7 Hz, 1H). |
| 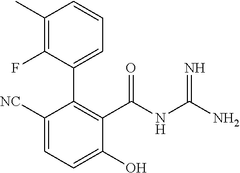 [SMILES: Cc1cccc(-c2c(C#N)ccc(O)c2C(=O)NC(=N)N)c1F] | N-carbamimidoyl-6-cyano-2′-fluoro-3-hydroxy- 3′-methyl-[1,1′-biphenyl]-2-carboxamide LC-MS: m/z 313 (M + H)⁺. 1H NMR (400 MHz, DMSO) δ 7.82 (br s, 2H), 7.63 (d, J = 8.8 Hz, 1H), 7.23 (t, J = 7.2 Hz, 1H), 7.07 (t, J = 7.6 Hz, 1H), 6.99 (t, J = 7.2 Hz, 1H), 6.87 (d, J = 8.8 Hz, 1H), 2.25 (s, 3H). |
| 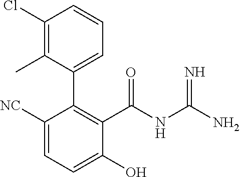 [SMILES: Cc1c(Cl)cccc1-c1c(C#N)ccc(O)c1C(=O)NC(=N)N] | N-carbamimidoyl-3′-chloro-6-cyano-3-hydroxy- 2′-methyl-[1,1′-biphenyl]-2-carboxamide LC-MS: m/z 329 (M + H)⁺. 1H NMR (400 MHz, DMSO) δ 7.59 (d, J = 8.7 Hz, 1H), 7.35 (d, J = 7.9 Hz, 1H), 7.18 (t, J = 7.7 Hz, 1H), 6.93 (d, J = 7.5 Hz, 1H), 6.82 (d, J = 8.7 Hz, 1H), 2.00 (s, 3H). |
| 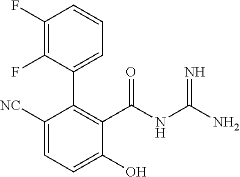 [SMILES: N#Cc1ccc(O)c(C(=O)NC(=N)N)c1-c1cccc(F)c1F] | N-carbamimidoyl-6-cyano-2′,3′-difluoro-3- hydroxy-[1,1′-biphenyl]-2-carboxamide LC-MS: m/z 317 (M + H)⁺. 1H NMR (400 MHz, DMSO) δ: 8.26 (br s, 1H), 7.67 (d, J = 8.8 Hz, 1H), 7.45-7.34 (m, 1H), 7.20 (dd, J = 12.6, 7.6 Hz, 1H), 7.03 (dd, J = 7.6, 6.3 Hz, 1H), 6.91 (d, J = 8.8 Hz, 1H). |
| 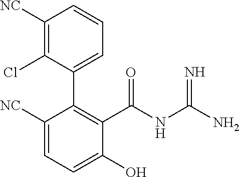 [SMILES: N#Cc1cccc(-c2c(C#N)ccc(O)c2C(=O)NC(=N)N)c1Cl] | N-carbamimidoyl-2′-chloro-3′,6-dicyano-3- hydroxy-[1,1′-biphenyl]-2-carboxamide LC-MS: m/z 340 (M + H)⁺. 1H NMR (400 MHz, DMSO) δ: 8.27 (br s, 1H), 7.96 (dd, J = 7.1, 2.2 Hz, 1H), 7.70 (d, J = 8.8 Hz, 1H), 7.63-7.54 (m, 2H), 7.26 (br s, 1H), 6.93 (d, J = 8.8 Hz, 1H). |
| 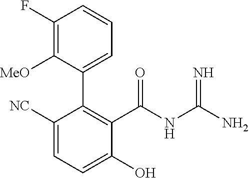 [SMILES: COc1c(F)cccc1-c1c(C#N)ccc(O)c1C(=O)NC(=N)N] | N-carbamimidoyl-6-cyano-3′-fluoro-3-hydroxy- 2′-methoxy-[1,1′-biphenyl]-2-carboxamide LC-MS: m/z 329 (M + H)⁺. 1H NMR (400 MHz, DMSO) δ 7.62 (d, J = 8.7 Hz, 1H), 7.20 (ddd, J = 11.9, 8.3, 1.5 Hz, 1H), 7.06 (td, J = 8.0, 5.1 Hz, 1H), 6.86 (d, J = 8.7 Hz, 2H), 3.63 (d, J = 1.4 Hz, 3H). |
| 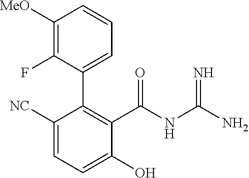 [SMILES: COc1cccc(-c2c(C#N)ccc(O)c2C(=O)NC(=N)N)c1F] | N-carbamimidoyl-6-cyano-2′-fluoro-3-hydroxy- 3′-methoxy-[1,1′-biphenyl]-2-carboxamide LC-MS: m/z 329 (M + H)⁺. 1H NMR (400 MHz, DMSO) δ 8.29 (br s, 2H), 7.63 (d, J = 8.7 Hz, 1H), 7.22 (br s, 1H), 7.12- 7.07 (m, 2H), 6.88 (d, J = 8.7 Hz, 1H), 6.74- 6.68 (m, 1H), 3.85 (s, 3H). |
| 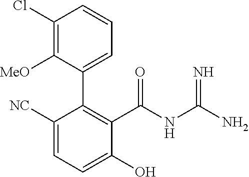 [SMILES: COc1c(Cl)cccc1-c1c(C#N)ccc(O)c1C(=O)NC(=N)N] | N-carbamimidoyl-3′-chloro-6-cyano-3-hydroxy- 2′-methoxy-[1,1′-biphenyl]-2-carboxamide LC-MS: m/z 345 (M + H)⁺. 1H NMR (400 MHz, DMSO) δ 8.21 (br s, 1H), 7.64 (d, J = 8.8 Hz, 1H), 7.42 (dd, J = 8.0, 1.6 Hz, 1H), 7.12 (t, J = 8.0 Hz, 1H), 7.03 (dd, J = 7.6, 1.6 Hz, 1H), 6.88 (d, J = 8.8 Hz, 1H), 3.49 (s, 3H). |
| 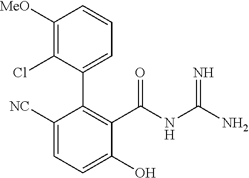 [SMILES: COc1cccc(-c2c(C#N)ccc(O)c2C(=O)NC(=N)N)c1Cl] | N-carbamimidoyl-2′-chloro-6-cyano-3-hydroxy- 3′-methoxy-[1,1′-biphenyl]-2-carboxamide LC-MS: m/z 345 (M + H)⁺. 1H NMR (400 MHz, DMSO) δ: 8.29 (br s, 2H), 7.62 (d, J = 8.7 Hz, 1H), 7.27 (br s, 1H), 7.07 (dd, J = 8.4, 1.3 Hz, 1H), 6.86 (d, J = 8.7 Hz, 1H), 6.75 (dd, J = 7.6, 1.3 Hz, 1H), 3.88 (s, 3H). |
| 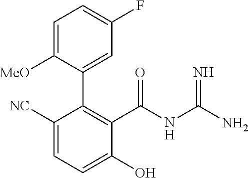 [SMILES: COc1ccc(F)cc1-c1c(C#N)ccc(O)c1C(=O)NC(=N)N] | N-carbamimidoyl-6-cyano-5′-fluoro-3-hydroxy- 2′-methoxy-[1,1′-biphenyl]-2-carboxamide LC-MS: m/z 329 (M + H)⁺. 1H NMR (400 MHz, DMSO) δ 8.23 (br s, 2H), 7.59 (d, J = 8.7 Hz, 1H), 7.09 (t, J = 8.5 Hz, 1H), 6.99 (dd, J = 8.8, 4.5 Hz, 1H), 6.87 (d, J = 8.7 Hz, 1H), 6.83 (d, J = 8.8 Hz, 1H), 3.64 (s, 3H). |
| 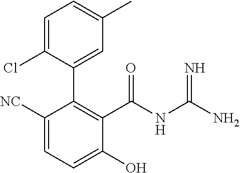 [SMILES: Cc1ccc(Cl)c(-c2c(C#N)ccc(O)c2C(=O)NC(=N)N)c1] | N-carbamimidoyl-2′-chloro-6-cyano-3-hydroxy- 5′-methyl-[1,1′-biphenyl]-2-carboxamide LC-MS: m/z 329 (M + H)⁺. 1H NMR (400 MHz, DMSO) δ 7.56 (d, J = 8.8 Hz, 1H), 7.30 (d, J = 8.0 Hz, 1H), 7.12 (d, J = 8.0 Hz, 1H), 6.98 (s, 1H), 6.79 (d, J = 8.0 Hz, 1H), 2.29 (s, 3H). |
| 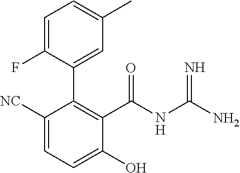 [SMILES: Cc1ccc(F)c(-c2c(C#N)ccc(O)c2C(=O)NC(=N)N)c1] | N-carbamimidoyl-6-cyano-2′-fluoro-3-hydroxy- 5′-methyl-[1,1′-biphenyl]-2-carboxamide LC-MS: m/z 313 (M + H)⁺. 1H NMR (400 MHz, DMSO) δ 7.80 (br s, 2H), 7.63 (d, J = 8.8 Hz, 1H), 7.19-7.12 (m, 1H), 7.05 (t, J = 9.2 Hz, 1H), 6.98 (d, J = 7.2 Hz, 1H), 6.87 (d, J = 8.4 Hz, 1H), 2.29 (s, 3H). |
| 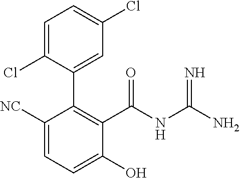 [SMILES: N#Cc1ccc(O)c(C(=O)NC(=N)N)c1-c1cc(Cl)ccc1Cl] | N-carbamimidoyl-2′,5′-dichloro-6-cyano-3- hydroxy-[1,1′-biphenyl]-2-carboxamide LC-MS: m/z 349 (M + H)⁺. 1H NMR (400 MHz, DMSO) δ 8.31 (br s, 2H), 7.66 (d, J = 8.8 Hz, 1H), 7.50 (d, J = 8.4 Hz, 1H), 7.41 (d, J = 8.8 Hz, 1H), 7.33 (s, 1H), 7.24 (br s, 1H), 6.89 (d, J = 8.8 Hz, 1H). |
| 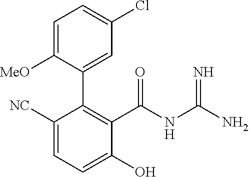 [SMILES: COc1ccc(Cl)cc1-c1c(C#N)ccc(O)c1C(=O)NC(=N)N] | N-carbamimidoyl-5′-chloro-6-cyano-3-hydroxy- 2′-methoxy-[1,1′-biphenyl]-2-carboxamide LC-MS: m/z 345 (M + H)⁺. 1H NMR (400 MHz, DMSO) δ 7.56 (d, J = 8.7 Hz, 1H), 7.31 (d, J = 8.9 Hz, 1H), 7.02 (d, J = 8.3 Hz, 2H), 6.79 (d, J = 8.7 Hz, 1H), 3.65 (s, 3H). |
| 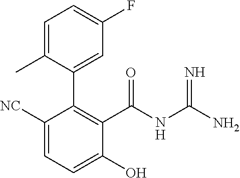 [SMILES: Cc1ccc(F)cc1-c1c(C#N)ccc(O)c1C(=O)NC(=N)N] | N-carbamimidoyl-6-cyano-5′-fluoro-3-hydroxy- 2′-methyl-[1,1′-biphenyl]-2-carboxamide LC-MS: m/z 313 (M + H)⁺. 1H NMR (400 MHz, DMSO) δ 7.79 (br s, 2H), 7.60 (d, J = 8.8 Hz, 1H), 7.21 (t, J = 7.6 Hz, 1H), 7.00 (t, J = 8.4 Hz, 1H), 6.82 (d, J = 9.2 Hz, 2H), 1.92 (s, 3H). |
| 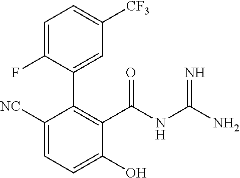 [SMILES: N#Cc1ccc(O)c(C(=O)NC(=N)N)c1-c1cc(C(F)(F)F)ccc1F] | N-carbamimidoyl-6-cyano-2′-fluoro-3-hydroxy- 5′-(trifluoromethyl)-[1,1′-biphenyl]-2- carboxamide LC-MS: m/z 367 (M + H)⁺. 1H NMR (400 MHz, DMSO) δ 7.81-7.77 (m, 1H), 7.69 (d, J = 8.8 Hz, 1H), 7.65 (dd, J = 6.4, 2.0 Hz, 1H), 7.46 (t, J = 8.8 Hz, 1H), 6.93 (d, J = 8.8 Hz, 1H). |
| 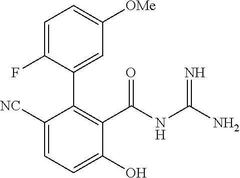 [SMILES: COc1ccc(F)c(-c2c(C#N)ccc(O)c2C(=O)NC(=N)N)c1] | N-carbamimidoyl-6-cyano-2′-fluoro-3-hydroxy- 5′-methoxy-[1,1′-biphenyl]-2-carboxamide LC-MS: m/z 329 (M + H)⁺. 1H NMR (400 MHz, DMSO) δ 8.29 (br s, 2H), 7.64 (d, J = 8.8 Hz, 1H), 7.10 (t, J = 8.9 Hz, 1H), 6.88 (d, J = 8.7 Hz, 2H), 6.73 (s, 1H), 3.73 (s, 3H). |
| 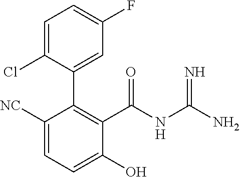 [SMILES: N#Cc1ccc(O)c(C(=O)NC(=N)N)c1-c1cc(F)ccc1Cl] | N-carbamimidoyl-2′-chloro-6-cyano-5′-fluoro-3- hydroxy-[1,1′-biphenyl]-2-carboxamide LC-MS: m/z 333 (M + H)⁺. 1H NMR (400 MHz, DMSO) δ 8.29 (br s, 2H), 7.64 (d, J = 8.0 Hz, 1H), 7.50 (dd, J = 8.8, 5.1 Hz, 1H), 7.22-7.14 (m, 2H), 6.89 (d, J = 8.8 Hz, 1H). |
| 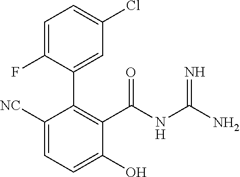 [SMILES: N#Cc1ccc(O)c(C(=O)NC(=N)N)c1-c1cc(Cl)ccc1F] | N-carbamimidoyl-5′-chloro-6-cyano-2′-fluoro-3- hydroxy-[1,1′-biphenyl]-2-carboxamide LC-MS: m/z 333 (M + H)⁺. 1H NMR (400 MHz, DMSO) δ: 7.66 (d, J = 8.8 Hz, 1H), 7.43 (ddd, J = 8.7, 4.4, 2.7 Hz, 1H), 7.32 (dd, J = 6.3, 2.7 Hz, 1H), 7.26 (t, J = 9.0 Hz, 1H), 6.90 (d, J = 8.8 Hz, 1H). |
| 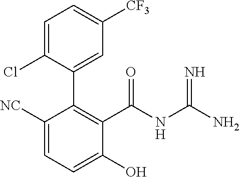 [SMILES: N#Cc1ccc(O)c(C(=O)NC(=N)N)c1-c1cc(C(F)(F)F)ccc1Cl] | N-carbamimidoyl-2′-chloro-6-cyano-3-hydroxy- 5′-(trifluoromethyl)-[1,1′-biphenyl]-2- carboxamide LC-MS: m/z 383 (M + H)⁺. 1H NMR (400 MHz, DMSO) δ 8.33 (br s, 2H), 7.73 (t, J = 4.3 Hz, 2H), 7.69 (d, J = 8.8 Hz, 1H), 7.61 (s, 1H), 7.32 (br s, 1H), 6.92 (d, J = 8.8 Hz, 1H). |
| 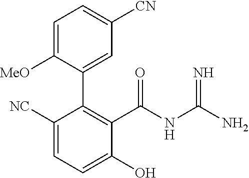 [SMILES: COc1ccc(C#N)cc1-c1c(C#N)ccc(O)c1C(=O)NC(=N)N] | N-carbamimidoyl-5′,6-dicyano-3-hydroxy-2′- methoxy-[1,1′-biphenyl]-2-carboxamide LC-MS: m/z 336 (M + H)⁺. 1H NMR (400 MHz, DMSO) δ 8.26 (br s, 2H), 7.80 (dd, J = 8.4, 2.0 Hz, 1H), 7.62 (d, J = 8.4 Hz, 1H), 7.49 (d, J = 2.4 Hz, 1H), 7.20 (d, J = 8.8 Hz, 1H), 6.86 (d, J = 8.4 Hz, 1H), 3.75 (s, 3H). |
| 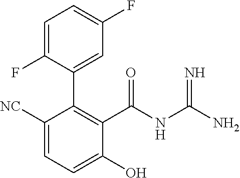 [SMILES: N#Cc1ccc(O)c(C(=O)NC(=N)N)c1-c1cc(F)ccc1F] | N-carbamimidoyl-6-cyano-2′,5′-difluoro-3- hydroxy-[1,1′-biphenyl]-2-carboxamide LC-MS: m/z 317 (M + H)⁺. 1H NMR (400 MHz, DMSO) δ: 8.32 (br s, 2H), 7.66 (d, J = 8.8 Hz, 1H), 7.32-7.10 (m, 3H), 6.90 (d, J = 8.8 Hz, 1H). |
| 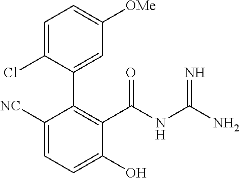 [SMILES: COc1ccc(Cl)c(-c2c(C#N)ccc(O)c2C(=O)NC(=N)N)c1] | N-carbamimidoyl-2′-chloro-6-cyano-3-hydroxy- 5′-methoxy-[1,1′-biphenyl]-2-carboxamide LC-MS: m/z 345 (M + H)⁺. 1H NMR (400 MHz, DMSO) δ 8.29 (br s, 2H), 7.64 (d, J = 8.7 Hz, 1H), 7.34 (d, J = 8.8 Hz, 1H), 6.94-6.84 (m, 2H), 6.76 (d, J = 3.0 Hz, 1H), 3.74 (s, 3H). |
| 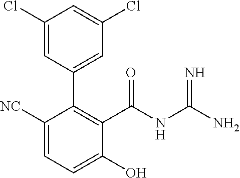 [SMILES: N#Cc1ccc(O)c(C(=O)NC(=N)N)c1-c1cc(Cl)cc(Cl)c1] | N-carbamimidoyl-3′,5′-dichloro-6-cyano-3- hydroxy-[1,1′-biphenyl]-2-carboxamide LC-MS: m/z 349 (M + H)⁺. 1H NMR (400 MHz, DMSO) δ 8.44 (br s, 2H), 7.56 (d, J = 8.8 Hz, 1H), 7.16 (t, J = 9.5 Hz, 1H), 6.93 (d, J = 7.4 Hz, 2H), 6.80 (d, J = 8.8 Hz, 1H). |
| 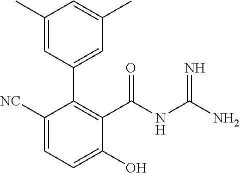 [SMILES: Cc1cc(C)cc(-c2c(C#N)ccc(O)c2C(=O)NC(=N)N)c1] | N-carbamimidoyl-6-cyano-3-hydroxy-3′,5′- dimethyl-[1,1′-biphenyl]-2-carboxamide LC-MS: m/z 309 (M + H)⁺. 1H NMR (400 MHz, DMSO) δ 8.17 (br s, 2H), 7.56 (d, J = 8.8 Hz, 1H), 7.20 (br s, 1H), 6.92 (s, 1H), 6.82 (d, J = 8.8 Hz, 1H), 6.74 (s, 2H), 2.27 (s, 6H). |
| 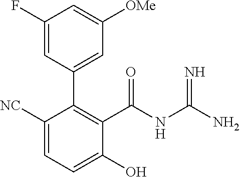 [SMILES: COc1cc(F)cc(-c2c(C#N)ccc(O)c2C(=O)NC(=N)N)c1] | N-carbamimidoyl-6-cyano-3′-fluoro-3-hydroxy- 5′-methoxy-[1,1′-biphenyl]-2-carboxamide LC-MS: m/z 329 (M + H)⁺. 1H NMR (400 MHz, DMSO) δ 8.08 (br s, 2H), 7.52 (d, J = 8.4 Hz, 1H), 6.85-6.68 (m, 2H), 6.57 (d, J = 10.4 Hz, 2H), 3.76 (s, 3H). |
| 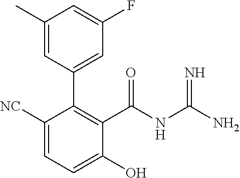 [SMILES: Cc1cc(F)cc(-c2c(C#N)ccc(O)c2C(=O)NC(=N)N)c1] | N-carbamimidoyl-6-cyano-3′-fluoro-3-hydroxy- 5′-methyl-[1,1′-biphenyl]-2-carboxamide LC-MS: m/z 313 (M + H)⁺. 1H NMR (400 MHz, DMSO) δ 7.56 (d, J = 8.7 Hz, 1H), 6.95 (d, J = 9.9 Hz, 1H), 6.84-6.75 (m, 3H), 2.32 (s, 3H). |
| 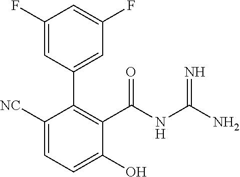 [SMILES: N#Cc1ccc(O)c(C(=O)NC(=N)N)c1-c1cc(F)cc(F)c1] | N-carbamimidoyl-6-cyano-3′,5′-difluoro-3- hydroxy-[1,1′-biphenyl]-2-carboxamide LC-MS: m/z 317 (M + H)⁺. 1H NMR (400 MHz, DMSO) δ 8.28 (s, 1H), 7.62 (d, J = 8.7 Hz, 1H), 7.56-7.52 (m, 1H), 7.28-7.21 (m, 2H), 6.87 (d, J = 8.7 Hz, 1H). |
| 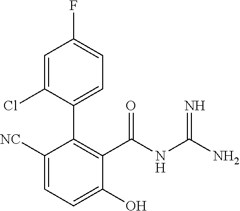 [SMILES: N#Cc1ccc(O)c(C(=O)NC(=N)N)c1-c1ccc(F)cc1Cl] | N-carbamimidoyl-2′-chloro-6-cyano-4′-fluoro-3- hydroxy-[1,1′-biphenyl-2-carboxamide LC-MS: m/z 333 (M + H)⁺. 1H NMR (400 MHz, DMSO) δ 7.81 (br s, 2H), 7.64 (d, J = 8.8 Hz, 1H), 7.45 (dd, J = 9.0, 2.3 Hz, 1H), 7.28-7.18 (m, 2H), 6.87 (d, J = 8.7 Hz, 1H). |
| 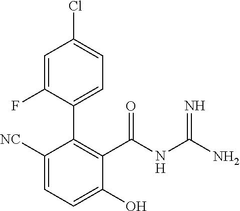 [SMILES: N#Cc1ccc(O)c(C(=O)NC(=N)N)c1-c1ccc(Cl)cc1F] | N-carbamimidoyl-4′-chloro-6-cyano-2′-fluoro-3- hydroxy-[1,1′-biphenyl]-2-carboxamide LC-MS: m/z 301 (M + H)⁺. 1H NMR (400 MHz, DMSO) δ 7.67 (d, J = 8.8 Hz, 1H), 7.44 (dd, J = 9.6, 1.8 Hz, 1H), 7.27 (br s, 2H), 6.90 (d, J = 8.8 Hz, 1H). |
| 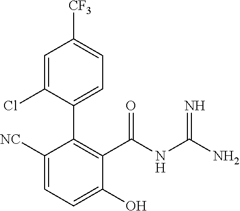 [SMILES: N#Cc1ccc(O)c(C(=O)NC(=N)N)c1-c1ccc(C(F)(F)F)cc1Cl] | N-carbamimidoyl-2′-chloro-6-cyano-3-hydroxy- 4′-(trifluoromethyl)-[1,1′-biphenyl]-2- carboxamide LC-MS: m/z 383 (M + H)⁺. 1H NMR (400 MHz, DMSO) δ 8.30 (br s, 2H), 7.91 (s, 1H), 7.73 (d, J = 7.6 Hz, 1H), 7.70 (d, J = 8.8 Hz, 1H), 7.47 (d, J = 8.0 Hz, 1H), 6.92 (d, J = 8.8 Hz, 1H). |
| 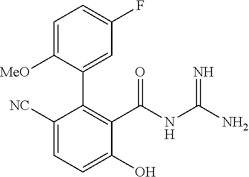 [SMILES: COc1ccc(F)cc1-c1c(C#N)ccc(O)c1C(=O)NC(=N)N] | N-carbamimidoyl-6-cyano-5′-fluoro-3-hydroxy- 2′-methoxy-[1,1′-biphenyl]-2-carboxamide LC-MS: m/z 329 (M + H)⁺. 1H NMR (400 MHz, DMSO) δ 8.23 (br s, 2H), 7.59 (d, J = 8.7 Hz, 1H), 7.09 (t, J = 8.5 Hz, 1H), 6.99 (dd, J = 8.8, 4.5 Hz, 1H), 6.87 (d, J = 8.7 Hz, 1H), 6.83 (d, J = 8.8 Hz, 1H), 3.64 (s, 3H). |
| 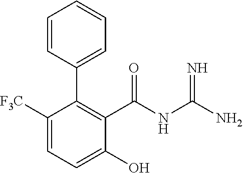 [SMILES: N=C(N)NC(=O)c1c(O)ccc(C(F)(F)F)c1-c1ccccc1] | N-carbamimidoyl-3-hydroxy-6- (trifluoromethyl)-[1,1′-biphenyl]-2-carboxamide LC-MS: m/z 324 (M + H)⁺. 1H NMR (400 MHz, DMSO) δ 8.18 (s, 1H), 7.99 (br s, 1H), 7.59 (d, J = 8.9 Hz, 1H), 7.32- 7.19 (m, 3H), 7.08 (d, J = 7.4 Hz, 2H), 6.92 (d, J = 8.8 Hz, 1H). |
| 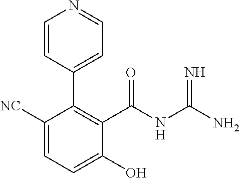 [SMILES: N#Cc1ccc(O)c(C(=O)NC(=N)N)c1-c1ccncc1] | N-carbamimidoyl-3-cyano-6-hydroxy-2- (pyridin-4-yl)benzamide LC-MS: m/z 282 (M + H)⁺. 1H NMR (400 MHz, DMSO-d6) δ 8.54 (dd, J = 4.5, 1.4 Hz, 2H), 7.61 (d, J = 8.8 Hz, 1H), 7.20 (dd, J = 4.5, 1.5 Hz, 2H), 6.85 (d, J = 8.8 Hz, 1H). |
| 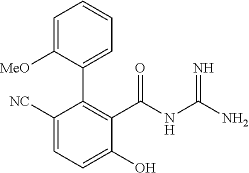 [SMILES: COc1ccccc1-c1c(C#N)ccc(O)c1C(=O)NC(=N)N] | N-carbamimidoyl-6-cyano-3-hydroxy-2′- methoxy-[1,1′-biphenyl]-2-carboxamide LC-MS: m/z 311 (M + H)⁺. 1H NMR (400 MHz, DMSO-d6) δ 8.20 (br s, 1H), 7.60 (d, J = 8.7 Hz, 1H), 7.29 (t, J = 7.0 Hz, 1H), 7.03-6.96 (m, 2H), 6.93 (t, J = 7.3 Hz, 1H), 6.84 (d, J = 8.6 Hz, 1H), 3.66 (s, 3H). |
| 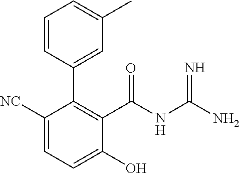 [SMILES: Cc1cccc(-c2c(C#N)ccc(O)c2C(=O)NC(=N)N)c1] | N-carbamimidoyl-6-cyano-3-hydroxy-3′-methyl- [1,1′-biphenyl]-2-carboxamide LC-MS: m/z 295 (M + H)⁺. 1H NMR (400 MHz, DMSO-d6) δ 7.56 (d, J = 8.7 Hz, 1H), 7.22 (t, J = 7.5 Hz, 1H), 7.11 (d, J = 7.6 Hz, 1H), 6.98-6.91 (m, 2H), 6.81 (d, J = 8.7 Hz, 1H), 2.31 (s, 3H). |
| 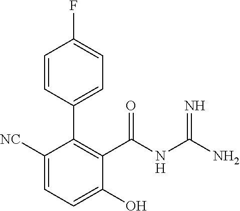 [SMILES: N#Cc1ccc(O)c(C(=O)NC(=N)N)c1-c1ccc(F)cc1] | N-carbamimidoyl-6-cyano-4′-fluoro-3-hydroxy- [1,1′-biphenyl]-2-carboxamide LC-MS: m/z 299 (M + H)⁺. 1H NMR (400 MHz, DMSO-d6) δ 7.56 (d, J = 8.7 Hz, 1H), 7.22-7.14 (m, 4H), 6.81 (d, J = 8.6 Hz, 1H). |
| 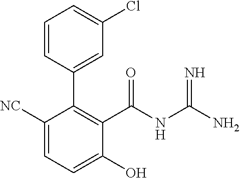 [SMILES: N#Cc1ccc(O)c(C(=O)NC(=N)N)c1-c1cccc(Cl)c1] | N-carbamimidoyl-3′-chloro-6-cyano-3-hydroxy- [1,1′-biphenyl]-2-carboxamide LC-MS: m/z 315 (M + H)⁺. 1H NMR (400 MHz DMSO-d6) δ 7.58 (d J = 8.8 Hz, 1H), 7.39-7.32 (m, 2H), 7.22 (s, 1H), 7.14-7.09 (m, 1H), 6.83 (d, J = 8.7 Hz, 1H). |
| 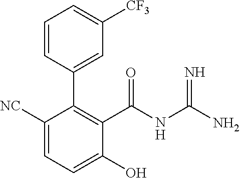 [SMILES: N#Cc1ccc(O)c(C(=O)NC(=N)N)c1-c1cccc(C(F)(F)F)c1] | N-carbamimidoyl-6-cyano-3-hydroxy-3′- (trifluoromethyl)-[1,1′-biphenyl]-2-carboxamide LC-MS: m/z 349 (M + H)⁺. 1H NMR (400 MHz, DMSO-d6) δ 7.68 (d, J = 7.7 Hz, 1H), 7.63-7.56 (m, 2H), 7.53-7.45 (m, 2H), 6.87 (d, J = 8.8 Hz, 1H). |
| 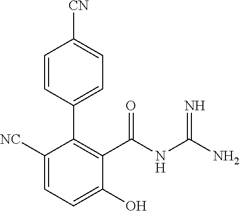 [SMILES: N#Cc1ccc(-c2c(C#N)ccc(O)c2C(=O)NC(=N)N)cc1] | N-carbamimidoyl-4′,6-dicyano-3-hydroxy-[1,1′- biphenyl]-2-carboxamide LC-MS: m/z 306 (M + H)⁺. 1H NMR (400 MHz, DMSO-d6) δ 7.81 (d, J = 8.3 Hz, 2H), 7.58 (d, J = 8.7 Hz, 1H), 7.42-7.33 (m, 2H), 6.82 (d, J = 8.7 Hz, 1H). |
| 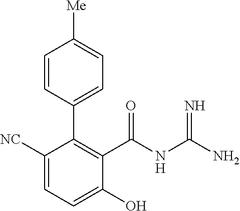 [SMILES: Cc1ccc(-c2c(C#N)ccc(O)c2C(=O)NC(=N)N)cc1] | N-carbamimidoyl-6-cyano-3-hydroxy-4′-methyl- [1,1′-biphenyl]-2-carboxamide LC-MS: m/z 295 (M + H)⁺. 1H NMR (400 MHz, DMSO-d6) δ 7.54 (d, J = 8.6 Hz, 1H), 7.13 (d, J = 7.9 Hz, 2H), 7.03 (d, J = 7.9 Hz, 2H), 6.78 (d, J = 8.5 Hz, 1H), 2.33 (s, 3H). |
| 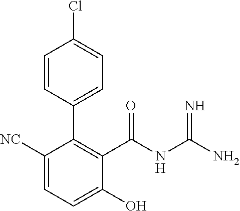 [SMILES: N#Cc1ccc(O)c(C(=O)NC(=N)N)c1-c1ccc(Cl)cc1] | N-carbamimidoyl-4′-chloro-6-cyano-3-hydroxy- [1,1′-biphenyl]-2-carboxamide LC-MS: m/z 315 (M + H)⁺. 1H NMR (400 MHz, DMSO-d6) δ 7.61 (d, J = 8.7 Hz, 1H), 7.40 (d, J = 8.3 Hz, 2H), 7.19 (d, J = 8.3 Hz, 2H), 6.86 (d, J = 8.7 Hz, 1H). |
| 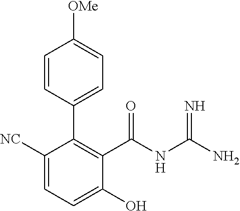 [SMILES: COc1ccc(-c2c(C#N)ccc(O)c2C(=O)NC(=N)N)cc1] | N-carbamimidoyl-6-cyano-3-hydroxy-4′- methoxy-[1,1′-biphenyl]-2-carboxamide LC-MS: m/z 311 (M + H)⁺. 1H NMR (400 MHz, DMSO-d6) δ 7.57 (d, J = 8.7 Hz, 1H), 7.08 (d, J = 8.5 Hz, 2H), 6.90 (d, J = 8.6 Hz, 2H), 6.82 (d, J = 8.7 Hz, 1H), 3.79 (s, 3H). |
| 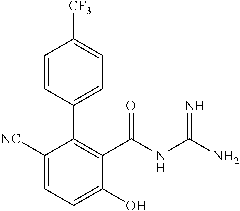 [SMILES: N#Cc1ccc(O)c(C(=O)NC(=N)N)c1-c1ccc(C(F)(F)F)cc1] | N-carbamimidoyl-6-cyano-3-hydroxy-4′- (trifluoromethyl)-[1,1′-biphenyl]-2-carboxamide LC-MS: m/z 349 (M + H)⁺. 1H NMR (400 MHz, DMSO-d6) δ 7.71 (d, J = 8.2 Hz, 2H), 7.63 (d, J = 8.8 Hz, 1H), 7.39 (d, J = 8.0 Hz, 2H), 6.88 (d, J = 8.8 Hz, 1H). |
| 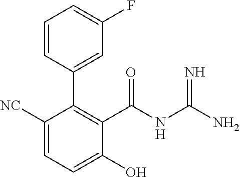 [SMILES: N#Cc1ccc(O)c(C(=O)NC(=N)N)c1-c1cccc(F)c1] | N-carbamimidoyl-6-cyano-3′-fluoro-3-hydroxy- [1,1′-biphenyl]-2-carboxamide LC-MS: m/z 299 (M + H)⁺. 1H NMR (400 MHz, DMSO-d6) δ 7.58 (d, J = 8.7 Hz, 1H), 7.38 (dd, J = 14.1, 7.9 Hz, 1H), 7.13 (td, J = 8.9, 2.5 Hz, 1H), 7.05-6.96 (m, 2H), 6.83 (d, J = 8.7 Hz, 1H). |
| 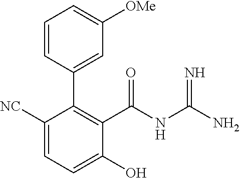 [SMILES: COc1cccc(-c2c(C#N)ccc(O)c2C(=O)NC(=N)N)c1] | N-carbamimidoyl-6-cyano-3-hydroxy-3′- methoxy-[1,1′-biphenyl]-2-carboxamide LC-MS: m/z 311 (M + H)⁺. 1H NMR (400 MHz, DMSO-d6) δ 7.55 (d, J = 8.7 Hz, 1H), 7.25 (t, J = 8.1 Hz, 1H), 6.89-6.83 (m, 1H), 6.80 (d, J = 8.7 Hz, 1H), 6.75-6.68 (m, 2H), 3.74 (s, 3H). |
| 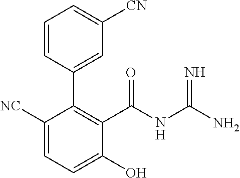 [SMILES: N#Cc1cccc(-c2c(C#N)ccc(O)c2C(=O)NC(=N)N)c1] | N-carbamimidoyl-3′,6-dicyano-3-hydroxy-[1,1′- biphenyl]-2-carboxamide LC-MS: m/z 306 (M + H)⁺. 1H NMR (400 MHz, DMSO-d6) δ 7.79 (d, J = 7.5 Hz, 1H), 7.69 (s, 1H), 7.63 (d, J = 8.7 Hz, 1H), 7.58-7.50 (m, 2H), 6.88 (d, J = 8.7 Hz, 1H). |
| 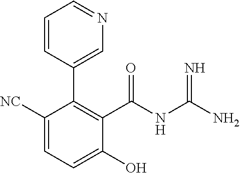 [SMILES: N#Cc1ccc(O)c(C(=O)NC(=N)N)c1-c1cccnc1] | N-carbamimidoyl-3-cyano-6-hydroxy-2- (pyridin-3-yl)benzamide LC-MS: m/z 282 (M + H)⁺. 1H NMR (400 MHz, DMSO-d6) δ 8.50 (d, J = 4.8 Hz, 1H), 8.35 (d, J = 2.1 Hz, 1H), 7.61 (d, J = 8.0 Hz, 2H), 7.42-7.34 (m, 1H), 6.85 (d, J = 8.8 Hz, 1H). |
| 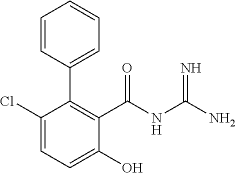 [SMILES: N=C(N)NC(=O)c1c(O)ccc(Cl)c1-c1ccccc1] | N-carbamimidoyl-6-chloro-3-hydroxy-[1,1′- biphenyl]-2-carboxamide LC-MS: m/z 290 (M + H)⁺. 1H NMR (400 MHz, DMSO) δ 15.36 (s, 1H), 8.02 (br s, 2H), 7.36 (d, J = 8.8 Hz, 1H), 7.33- 7.27 (m, 2H), 7.26-7.20 (m, 1H), 7.05-7.01 (m, 2H), 6.84 (d, J = 8.8 Hz, 1H). |
| 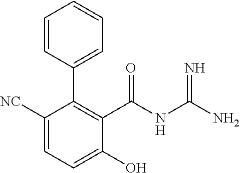 [SMILES: N#Cc1ccc(O)c(C(=O)NC(=N)N)c1-c1ccccc1] | N-carbamimidoyl-6-cyano-3-hydroxy-[1,1′- biphenyl]-2-carboxamide LC-MS: m/z 281 (M + H)⁺. 1H NMR (400 MHz, DMSO) δ 12.44 (br s, 1H), 8.27-8.12 (m, 4H), 7.88 (d, J = 8.8 Hz, 1H), 7.48-7.44 (m, 3H), 7.35-7.31 (m, 2H), 7.12 (d, J = 8.8 Hz, 1H). |

### General Procedure 8

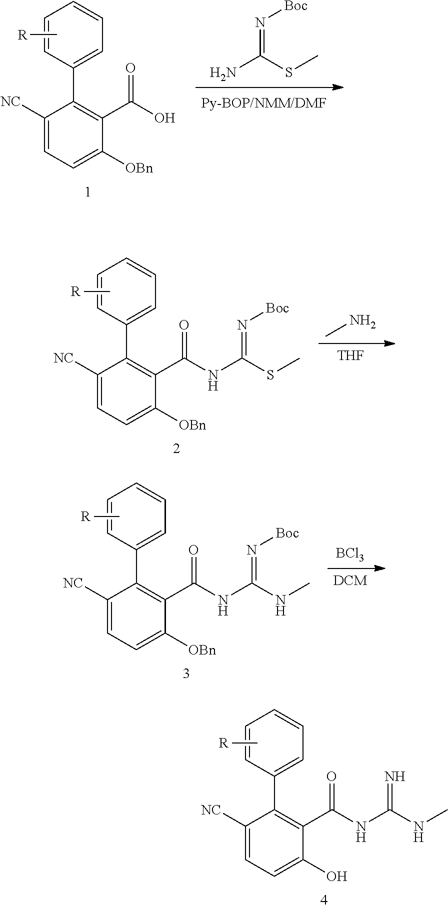

### Step 1: Synthesis of Compound 2

The synthesis of compound 2 was described in the general procedure 5.

### Step 2: Synthesis of Compound 3

Et₃N (5 eq.) was added to a solution of 2 (1 eq.) in THF followed by methylamine (3 eq.) and the resulting mixture was stirred at room temperature for 1 hour. The mixture was then poured into water and extracted with EtOAc. The organic layer was dried over anhydrous Na₂SO₄, filtered and concentrated under vacuum. The residue was purified by column chromatography on silica gel (eluted with Petroleum Ether:EA=3:1 to 1:1) to give compound 3.

### Step 3: Synthesis of Compound 4

BCl₃ (10 eq.) was added at −78° C. under N₂ atmosphere to a solution of 3 (1 eq.) in DCM and the resulting mixture was slowly warmed to 0° C. and stirred for 1 hour. The mixture was then quenched with MeOH (0.5 mL) and the solvent was removed under vacuum. The residue was purified via prep-HPLC (C18, 0% to 50% acetonitrile in H₂O with 0.1% NH₃.H₂O) to give compound 4.

### Synthesis of A365

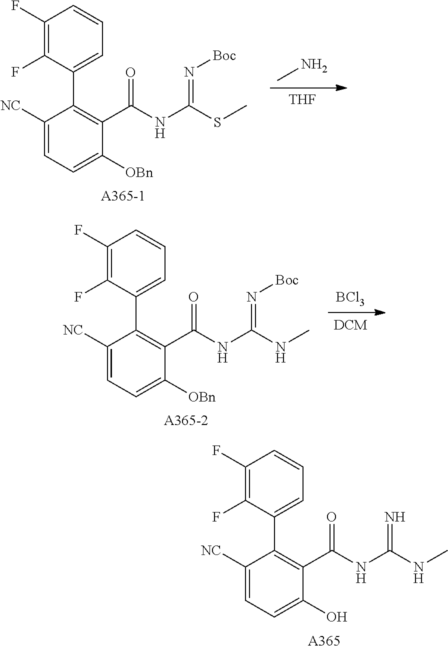

### Step 1: Synthesis of Compound A365-2

Et₃N (0.065 mL, 0.465 mmol) was added to a solution of A365-1 (50 mg, 0.0930 mmol) in THF (3 mL) followed by methylamine hydrochloride (19 mg, 0.279 mmol). The resulting mixture was stirred at room temperature for 1 hour. The mixture was then poured into water and extracted with EtOAc twice. The combined organic layers were dried over anhydrous Na₂SO₄, filtered and concentrated under vacuum. The residue was purified by column chromatography on silica gel (eluted with Petroleum Ether:EA=3:1 to 1:1) to give A365-2 (30 mg, 61.9719% yield) as a white solid. LC/MS (ESI) m/z: 521 (M+H)⁺.

### Step 3: Synthesis of Compound A365

BCl₃ (0.5760 mL, 1 M in DCM) was added at −78° C. under N₂ atmosphere to a solution of A365-2 (30 mg, 0.0576 mmol) in DCM (3 mL), and the resulting mixture was slowly warmed to 0° C. and stirred for 1 hour. The mixture was then quenched with MeOH (0.5 mL) and the solvent was removed under vacuum. The residue was purified via prep-HPLC (C18, 0% to 50% acetonitrile in H₂O with 0.1% NH₃.H₂O) to give A365 (3.5 mg, 18.4% yield) as a white solid. LC/MS (ESI) m/z: 331 (M+H)⁺. 1H NMR (400 MHz, DMSO) δ: 7.56 (d, J=8.8 Hz, 1H), 7.28-7.09 (m, 2H), 7.00-6.85 (m, 2H), 2.89 (d, J=37.6 Hz, 3H).

The compounds in the table below were prepared from the appropriate starting materials described previously or commercially available using the above general procedure 8.

| Structure | Name |
|---|---|
| 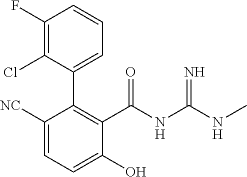 [SMILES: CNC(=N)NC(=O)c1c(O)ccc(C#N)c1-c1cccc(F)c1Cl] | 2′-chloro-6-cyano-3′-fluoro-3-hydroxy-N-(N- methylcarbamimidoyl)-[1,1′-biphenyl]-2- carboxamide LC-MS: m/z 347 (M + H)⁺. 1H NMR (400 MHz, DMSO) δ 8.36 (br s, 2H), 7.67 (d, J = 8.8 Hz, 1H), 7.41-7.32 (m, 2H), 7.09-6.99 (m, 1H), 6.91 (d, J = 8.7 Hz, 1H), 2.75 (s, 3H). |
| 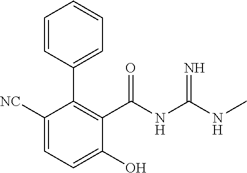 [SMILES: CNC(=N)NC(=O)c1c(O)ccc(C#N)c1-c1ccccc1] | 6-cyano-3-hydroxy-N-(N-methylcarbamimidoyl)- [1,1′-biphenyl]-2-carboxamide LC-MS: m/z 295 (M + H)⁺. 1H NMR (400 MHz, DMSO) δ 7.61 (d, J = 8.6 Hz, 1H), 7.39-7.29 (m, 3H), 7.16 (d, J = 6.7 Hz, 2H), 6.87 (d, J = 8.7 Hz, 1H), 2.73 (s, 3H). |
| 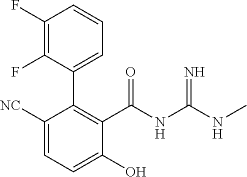 [SMILES: CNC(=N)NC(=O)c1c(O)ccc(C#N)c1-c1cccc(F)c1F] | 6-cyano-2′,3′-difluoro-3-hydroxy-N-(N- methylcarbamimidoyl)-[1,1′-biphenyl]-2- carboxamide LC-MS: m/z 331 (M + H)⁺. 1H NMR (400 MHz, DMSO) δ: 7.56 (d, J = 8.8 Hz, 1H), 7.28-7.09 (m, 2H), 7.00-6.85 (m, 2H), 2.89 (s, 3H). |
| 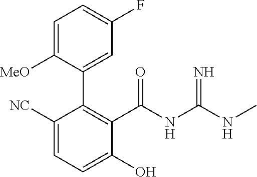 [SMILES: CNC(=N)NC(=O)c1c(O)ccc(C#N)c1-c1cc(F)ccc1OC] | 6-cyano-5′-fluoro-3-hydroxy-2′-methoxy-N-(N- methylcarbamimidoyl)-[1,1′-biphenyl]-2- carboxamide LC-MS: m/z 343 (M + H)⁺. 1H NMR (400 MHz, DMSO) δ 7.61 (d, J = 8.4 Hz, 1H), 7.10 (td, J = 8.7, 3.1 Hz, 1H), 7.00 (dd, J = 9.0, 4.6 Hz, 1H), 6.87 (dd, J = 12.5, 6.3 Hz, 2H), 3.64 (s, 3H), 2.72 (s, 3H). |
| 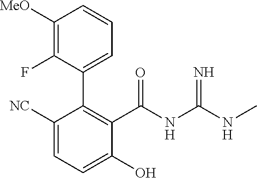 [SMILES: CNC(=N)NC(=O)c1c(O)ccc(C#N)c1-c1cccc(OC)c1F] | 6-cyano-2′-fluoro-3-hydroxy-3′-methoxy-N-(N- methylcarbamimidoyl)-[1,1′-biphenyl]-2- carboxamide LC-MS: m/z 343 (M + H)⁺. 1H NMR (400 MHz, DMSO) δ 8.96 (s, 1H), 8.31 (s, 1H), 7.64 (d, J = 8.6 Hz, 1H), 7.10 (d, J = 5.4 Hz, 2H), 6.89 (d, J = 8.9 Hz, 1H), 6.76-6.67 (m, 1H), 3.85 (s, 3H), 2.74 (s, 3H). |
| 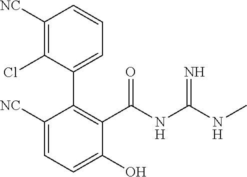 [SMILES: CNC(=N)NC(=O)c1c(O)ccc(C#N)c1-c1cccc(C#N)c1Cl] | 2′-chloro-3′,6-dicyano-3-hydroxy-N-(N- methylcarbamimidoyl)-[1,1′-biphenyl]-2- carboxamide LC-MS: m/z 354 (M + H)⁺. 1H NMR (400 MHz, DMSO) δ 7.96 (dd, J = 7.0, 2.3 Hz, 1H), 7.70 (d, J = 8.7 Hz, 1H), 7.62-7.54 (m, 2H), 6.93 (d, J = 8.8 Hz, 1H), 2.74 (s, 3H). |
| 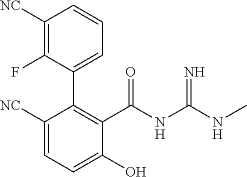 [SMILES: CNC(=N)NC(=O)c1c(O)ccc(C#N)c1-c1cccc(C#N)c1F] | 3′,6-dicyano-2′-fluoro-3-hydroxy-N-(N- methylcarbamimidoyl)-[1,1′-biphenyl]-2- carboxamide LC-MS: m/z 338 (M + H)⁺. 1H NMR (400 MHz, DMSO) δ 8.95 (s, 1H), 7.92 (t, J = 6.3 Hz, 1H), 7.71 (d, J = 8.8 Hz, 1H), 7.64 (t, J = 6.8 Hz, 1H), 7.44 (t, J = 7.7 Hz, 1H), 6.95 (d, J = 8.8 Hz, 1H), 2.75 (s, 3H). |
| 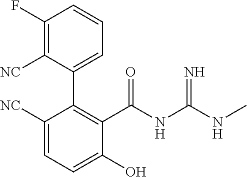 [SMILES: CNC(=N)NC(=O)c1c(O)ccc(C#N)c1-c1cccc(F)c1C#N] | 2′,6-dicyano-3′-fluoro-3-hydroxy-N-(N- methylcarbamimidoyl)-[1,1′-biphenyl]-2- carboxamide LC-MS: m/z 338 (M + H)⁺. 1H NMR (400 MHz, DMSO) δ: 8.96 (s, 1H), 8.48 (s, 1H), 7.85-7.70 (m, 2H), 7.51 (t, J = 8.9 Hz, 1H), 7.26 (d, J = 7.7 Hz, 1H), 6.97 (d, J = 8.8 Hz, 1H), 2.79 (s, 3H). |
| 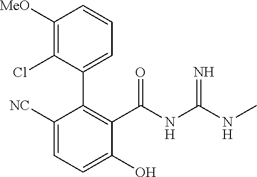 [SMILES: CNC(=N)NC(=O)c1c(O)ccc(C#N)c1-c1cccc(OC)c1Cl] | 2′-chloro-6-cyano-3-hydroxy-3′-methoxy-N-(N- methylcarbamimidoyl)-[1,1′-biphenyl]-2- carboxamide LC-MS: m/z 359 (M + H)⁺. 1H NMR (400 MHz, DMSO) δ 8.95 (s, 1H), 8.44 (br s, 1H), 7.63 (d, J = 8.4 Hz, 1H), 7.28 (t, J = 8.0 Hz, 1H), 7.08 (d, J = 8.0 Hz, 1H), 6.88 (d, J = 8.8 Hz, 1H), 6.75 (d, J = 6.8 Hz, 1H), 3.88 (s, 3H), 2.74 (s, 3H). |

### General Procedure 9

Combination of the general procedures 3 and 5 described above.

### Synthesis of A26

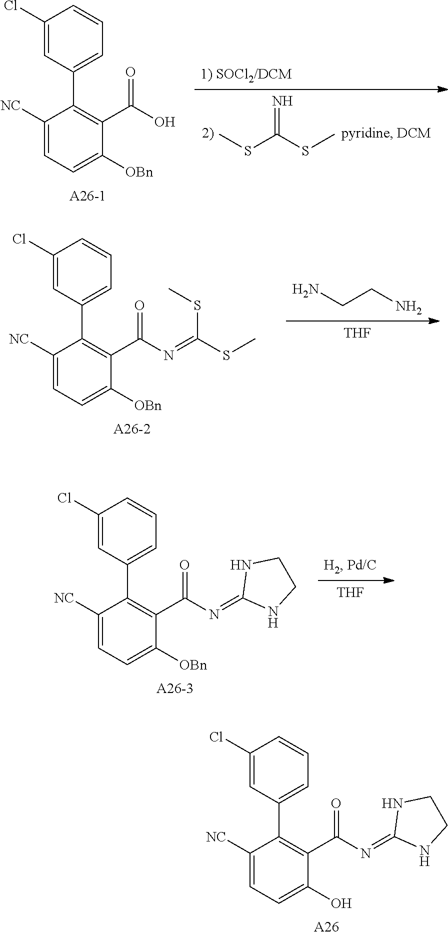

### Step 1: Synthesis of Compound A26-2

Thionyl chloride (2 mL, 27.57 mmol) was added to a solution of A26-1 (135 mg, 0.37 mmol) in DCM (10 mL) and the mixture was stirred 65° C. for 2 hours. The solvent was then removed under vacuum and the crude acyl chloride was dissolved in anhydrous DCM (2 mL) and added to a mixture of bis(methylsulfanyl)methanimine (67.4854 mg, 0.5567 mmol) and pyridine (494 mg, 1.86 mmol) in DCM (10 mL) at 0° C. The resulting mixture was stirred at room temperature for 30 minutes. The solvent was then removed under vacuum. The residue was purified by column chromatography on silica gel (eluted with Petroleum Ether:EA=10:1 to 1:1) to give A26-2 (83 mg, 47.9% yield) as a light yellow solid. LC/MS (ESI) m/z: 467 (M+H)⁺.

### Step 2: Synthesis of Compound A26-3

Ethane-1,2-diamine (0.048 mL, 0.71 mmol) was added to a solution of A26-2 (83 mg, 0.18 mmol) in THF (3 mL) and EtOH (3 mL), and the resulting mixture was heated to 80° C. for 1 hour. The solvents were then removed under vacuum and the residue was recrystallized from MeOH to give A26-3 (53 mg, 69.22% yield) as a white solid. LC/MS (ESI) m/z: 431 (M+H)⁺.

### Step 3: Synthesis of Compound A26-4

10% Pd/C (53 mg) was added under N₂ atmosphere to a solution of A26-3 (53 mg, 0.123 mmol) in THF (4 mL), and the resulting mixture was stirred at room temperature for 30 minutes under 15 psi H₂. The mixture was then filtered and the filtrate was concentrated under vacuum. The residue was purified via prep-HPLC (C18, 0% to 50% acetonitrile in H₂O with 0.1% NH₃.H₂O) to give A26 (14 mg, 33.4% yield) as a white solid. LC-MS: m/z 341 (M+H)⁺0.1H NMR (400 MHz, DMSO) δ: 8.55 (s, 2H), 7.68 (d, J=8.7 Hz, 1H), 7.43-7.34 (m, 2H), 7.25 (s, 1H), 7.16-7.11 (m, 1H), 6.95 (d, J=8.7 Hz, 1H), 3.53 (s, 4H).

The compounds in the table below were prepared from the appropriate starting materials described previously or commercially available using the above general procedure 9.

| Structure | Name |
|---|---|
| 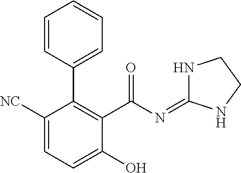 [SMILES: N#Cc1ccc(O)c(C(=O)N=C2NCCN2)c1-c1ccccc1] | 6-cyano-3-hydroxy-N-(imidazolidin-2-ylidene)- [1,1′-biphenyl]-2-carboxamide LC-MS: m/z 307 (M + H)⁺. 1H NMR (400 MHz, MeOD) δ 8.41 (br s, 2H), 7.58 (d, J = 8.4 Hz, 1H), 7.42-7.27 (m, 3H), 7.20 (d, J = 7.2 Hz, 2H), 6.93 (d, J = 8.8 Hz, 1H), 3.64 (s, 4H). |
| 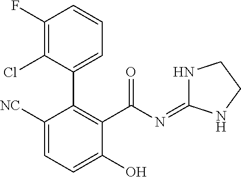 [SMILES: N#Cc1ccc(O)c(C(=O)N=C2NCCN2)c1-c1cccc(F)c1Cl] | 2′-chloro-6-cyano-3′-fluoro-3-hydroxy-N- (imidazolidin-2-ylidene)-[1,1′-biphenyl]-2- carboxamide LC-MS: m/z 359 (M + H)⁺. 1H NMR (400 MHz, DMSO) δ 8.57 (s, 2H), 7.73 (d, J = 8.7 Hz, 1H), 7.40-7.32 (m, 2H), 7.07 (d, J = 6.1 Hz, 1H), 6.98 (d, J = 8.7 Hz, 1H), 3.52 (s, 4H). |
| 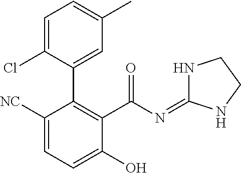 [SMILES: Cc1ccc(Cl)c(-c2c(C#N)ccc(O)c2C(=O)N=C2NCCN2)c1] | 2′-chloro-6-cyano-3-hydroxy-N-(imidazolidin-2- ylidene)-5′-methyl-[1,1′-biphenyl]-2- carboxamide LC-MS: m/z 355 (M + H)⁺. 1H NMR (400 MHz, DMSO) δ 8.52 (s, 2H), 7.69 (d, J = 8.4 Hz, 1H), 7.33 (d, J = 8.0 Hz, 1H), 7.14 (dd, J = 8.0, 1.6 Hz, 1H), 7.01 (d, J = 2.0 Hz, 1H), 6.95 (d, J = 8.8 Hz, 1H), 3.52 (s, 4H), 2.29 (s, 3H). |
| 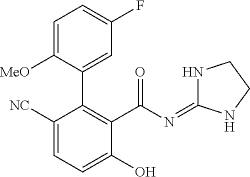 [SMILES: COc1ccc(F)cc1-c1c(C#N)ccc(O)c1C(=O)N=C1NCCN1] | 6-cyano-5′-fluoro-3-hydroxy-N-(imidazolidin-2- ylidene)-2′-methoxy-[1,1′-biphenyl]-2- carboxamide LC-MS: m/z 355 (M + H)⁺. 1H NMR (400 MHz, DMSO) δ 8.48 (s, 2H), 7.65 (d, J = 8.7 Hz, 1H), 7.09 (td, J = 8.7, 3.1 Hz, 1H), 7.00 (dd, J = 9.0, 4.6 Hz, 1H), 6.94-6.85 (m, 2H), 3.64 (s, 3H), 3.52 (s, 4H). |
| 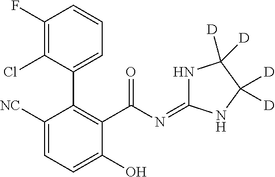 [SMILES: [2H]C1([2H])NC(=NC(=O)c2c(O)ccc(C#N)c2-c2cccc(F)c2Cl)NC1([2H])[2H]] | LC-MS: m/z 363 (M + H)⁺. 1H NMR (400 MHz, DMSO) δ 8.55 (s, 2H), 7.73 (d, J = 8.7 Hz, 1H), 7.42-7.32 (m, 2H), 7.09-7.03 (m, 1H), 6.98 (d, J = 8.7 Hz, 1H). |
| 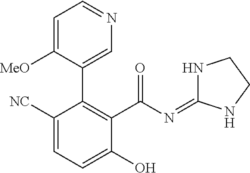 [SMILES: COc1ccncc1-c1c(C#N)ccc(O)c1C(=O)N=C1NCCN1] | 3-cyano-6-hydroxy-N-(imidazolidin-2-ylidene)- 2-(4-methoxypyridin-3-yl)benzamide LC-MS: m/z 338 (M + H)⁺. 1H NMR (400 MHz, DMSO) δ 8.52 (s, 2H), 8.42 (d, J = 6.0 Hz, 1H), 8.05 (s, 1H), 7.68 (d, J = 8.8 Hz, 1H), 7.09 (d, J = 5.6 Hz, 1H), 6.94 (d, J = 8.4 Hz, 1H), 3.75 (s, 3H), 3.53 (s, 4H). |
| 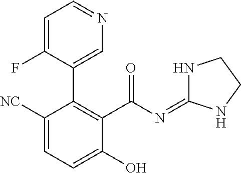 [SMILES: N#Cc1ccc(O)c(C(=O)N=C2NCCN2)c1-c1cnccc1F] | 3-cyano-2-(4-fluoropyridin-3-yl)-6-hydroxy-N- (imidazolidin-2-ylidene)benzamide LC-MS: m/z 326 (M + H)⁺. 1H NMR (400 MHz, DMSO) δ 8.52 (br s, 2H), 8.41 (d, J = 5.0 Hz, 1H), 7.78-7.68 (m, 2H), 7.50-7.43 (m, 1H), 7.03 (d, J = 8.7 Hz, 1H), 3.54 (s, 4H). |
| 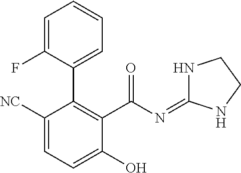 [SMILES: N#Cc1ccc(O)c(C(=O)N=C2NCCN2)c1-c1ccccc1F] | 6-cyano-2′-fluoro-3-hydroxy-N-(imidazolidin-2- ylidene)-[1,1′-biphenyl]-2-carboxamide LC-MS: m/z 325 (M + H)⁺. 1H NMR (400 MHz, DMSO) δ 8.54 (s, 2H), 7.71 (d, J = 8.7 Hz, 1H), 7.41-7.33 (m, 1H), 7.23-7.15 (m, 3H), 6.96 (d, J = 8.7 Hz, 1H), 3.52 (s, 4H). |
| 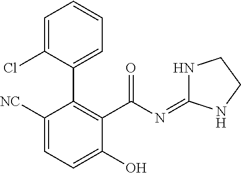 [SMILES: N#Cc1ccc(O)c(C(=O)N=C2NCCN2)c1-c1ccccc1Cl] | 2′-chloro-6-cyano-3-hydroxy-N-(imidazolidin-2- ylidene)-[1,1′-biphenyl]-2-carboxamide LC-MS: m/z 341 (M + H)⁺. 1H NMR (400 MHz, DMSO) δ 8.36 (br s, 2H), 7.65 (d, J = 8.7 Hz, 1H), 7.49-7.41 (m, 1H), 7.35-7.29 (m, 2H), 7.21-7.15 (m, 1H), 6.90 (d, J = 8.7 Hz, 1H), 3.49 (s, 4H). |
| 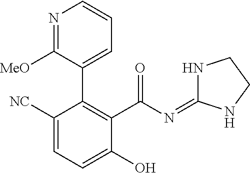 [SMILES: COc1ncccc1-c1c(C#N)ccc(O)c1C(=O)N=C1NCCN1] | 3-cyano-6-hydroxy-N-(imidazolidin-2-ylidene)- 2-(2-methoxypyridin-3-yl)benzamide LC-MS: m/z 338 (M + H)⁺. 1H NMR (400 MHz, DMSO) δ 8.50 (s, 2H), 8.11 (dd, J = 5.0, 1.9 Hz, 1H), 7.67 (d, J = 8.7 Hz, 1H), 7.41 (dd, J = 7.2, 1.9 Hz, 1H), 6.99 (dd, J = 7.2, 5.1 Hz, 1H), 6.91 (d, J = 8.7 Hz, 1H), 3.74 (s, 3H), 3.51 (s, 4H). |
| 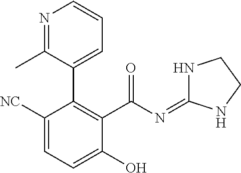 [SMILES: Cc1ncccc1-c1c(C#N)ccc(O)c1C(=O)N=C1NCCN1] | 3-cyano-6-hydroxy-N-(imidazolidin-2-ylidene)- 2-(2-methylpyridin-3-yl)benzamide LC-MS: m/z 322 (M + H)⁺. 1H NMR (400 MHz, DMSO) δ 8.54 (s, 2H), 8.38 (dd, J = 4.9, 1.7 Hz, 1H), 7.72 (d, J = 8.7 Hz, 1H), 7.37 (dd, J = 7.6, 1.7 Hz, 1H), 7.21 (dd, J = 7.6, 4.9 Hz, 1H), 6.96 (d, J = 8.7 Hz, 1H), 3.52 (s, 4H), 2.15 (s, 3H). |
| 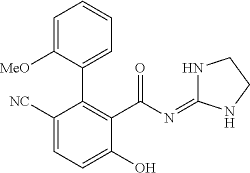 [SMILES: COc1ccccc1-c1c(C#N)ccc(O)c1C(=O)N=C1NCCN1] | 6-cyano-3-hydroxy-N-(imidazolidin-2-ylidene)- 2′-methoxy-[1,1′-biphenyl]-2-carboxamide LC-MS: m/z 427 (M + H)⁺. 1H NMR (400 MHz, DMSO) δ 8.48 (s, 2H), 7.63 (d, J = 8.6 Hz, 1H), 7.32-7.19 (m, 1H), 7.06-6.78 (m, 4H), 3.65 (s, 3H), 3.51 (s, 4H). |
| 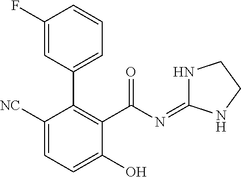 [SMILES: N#Cc1ccc(O)c(C(=O)N=C2NCCN2)c1-c1cccc(F)c1] | 6-cyano-3′-fluoro-3-hydroxy-N-(imidazolidin-2- ylidene)-[1,1′-biphenyl]-2-carboxamide LC-MS: m/z 325 (M + H)⁺. 1H NMR (400 MHz, DMSO) δ 8.54 (s, 2H), 7.67 (d, J = 8.7 Hz, 1H), 7.39 (td, J = 7.9, 6.2 Hz, 1H), 7.13 (td, J = 8.4, 2.2 Hz, 1H), 7.05-6.99 (m, 2H), 6.94 (d, J = 8.7 Hz, 1H), 3.52 (s, 4H). |
| 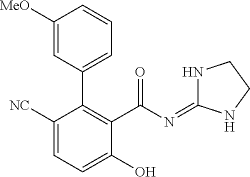 [SMILES: COc1cccc(-c2c(C#N)ccc(O)c2C(=O)N=C2NCCN2)c1] | 6-cyano-3-hydroxy-N-(imidazolidin-2-ylidene)- 3′-methoxy-[1,1′-biphenyl]-2-carboxamide LC-MS: m/z 337 (M + H)⁺. 1H NMR (400 MHz, DMSO) δ 8.52 (s, 2H), 7.64 (d, J = 8.7 Hz, 1H), 7.33-7.20 (m, 1H), 6.92 (d, J = 8.7 Hz, 2H), 6.87 (ddd, J = 8.3, 2.5, 0.9 Hz, 2H), 3.74 (s, 3H), 3.51 (s, 4H). |
| 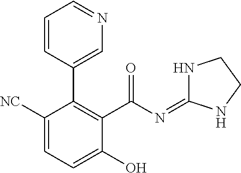 [SMILES: N#Cc1ccc(O)c(C(=O)N=C2NCCN2)c1-c1cccnc1] | 3-cyano-6-hydroxy-N-(imidazolidin-2-ylidene)- 2-(pyridin-3-yl)benzamide LC-MS: m/z 308 (M + H)⁺. 1H NMR (400 MHz, DMSO) δ 8.54 (br s, 2H), 8.51 (dd, J = 4.8, 1.6 Hz, 3H), 8.37 (d, J = 1.6 Hz, 1H), 7.71 (d, J = 8.7 Hz, 1H), 7.68-7.55 (m, 1H), 7.46-7.33 (m, 1H), 6.98 (d, J = 8.7 Hz, 1H), 3.52 (s, 4H). |
| 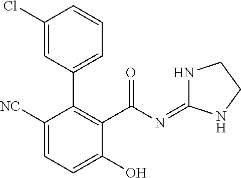 [SMILES: N#Cc1ccc(O)c(C(=O)N=C2NCCN2)c1-c1cccc(Cl)c1] | 3′-chloro-6-cyano-3-hydroxy-N-(imidazolidin-2- ylidene)-[1,1′-biphenyl]-2-carboxamide LC-MS: m/z 341 (M + H)⁺. 1H NMR (400 MHz, DMSO) δ: 8.55 (s, 2H), 7.68 (d, J = 8.7 Hz, 1H), 7.43-7.34 (m, 2H), 7.25 (s, 1H), 7.16-7.11 (m, 1H), 6.95 (d, J = 8.7 Hz, 1H), 3.53 (s, 4H). |
| 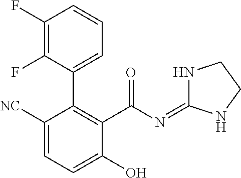 [SMILES: N#Cc1ccc(O)c(C(=O)N=C2NCCN2)c1-c1cccc(F)c1F] | 6-cyano-2′,3′-difluoro-3-hydroxy-N- (imidazolidin-2-ylidene)-[1,1′-biphenyl]-2- carboxamide LC-MS: m/z 343 (M + H)⁺. 1H NMR (400 MHz, DMSO) δ: 8.58 (s, 2H), 7.74 (d, J = 8.7 Hz, 1H), 7.45-7.31 (m, 1H), 7.21 (dd, J = 12.9, 8.0 Hz, 1H), 7.09-6.97 (m, 2H), 3.54 (s, 4H). |
| 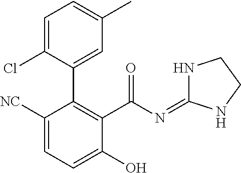 [SMILES: Cc1ccc(Cl)c(-c2c(C#N)ccc(O)c2C(=O)N=C2NCCN2)c1] | 2′-chloro-6-cyano-3-hydroxy-N-(imidazolidin-2- ylidene)-5′-methyl-[1,1′-biphenyl]-2- carboxamide LC-MS: m/z 355 (M + H)⁺. 1H NMR (400 MHz, DMSO) δ 8.52 (s, 2H), 7.69 (d, J = 8.4 Hz, 1H), 7.33 (d, J = 8.0 Hz, 1H), 7.14 (dd, J = 8.0, 1.6 Hz, 1H), 7.01 (d, J = 2.0 Hz, 1H), 6.95 (d, J = 8.8 Hz, 1H), 3.52 (s, 4H), 2.29 (s, 3H). |
| 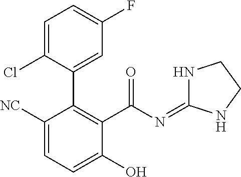 [SMILES: N#Cc1ccc(O)c(C(=O)N=C2NCCN2)c1-c1cc(F)ccc1Cl] | 2′-chloro-6-cyano-5′-fluoro-3-hydroxy-N- (imidazolidin-2-ylidene)-[1,1′-biphenyl]-2- carboxamide LC-MS: m/z 359 (M + H)⁺. 1H NMR (400 MHz, DMSO) δ 8.55 (s, 2H), 7.73 (d, J = 8.7 Hz, 1H), 7.51 (dd, J = 8.7, 5.1 Hz, 1H), 7.24-7.17 (m, 2H), 6.98 (d, J = 8.7 Hz, 1H), 3.54 (s, 4H). |
| 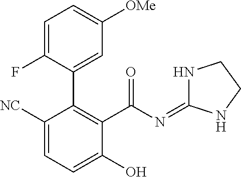 [SMILES: COc1ccc(F)c(-c2c(C#N)ccc(O)c2C(=O)N=C2NCCN2)c1] | 6-cyano-2′-fluoro-3-hydroxy-N-(imidazolidin-2- ylidene)-5′-methoxy-[1,1′-biphenyl]-2- carboxamide LC-MS: m/z 355 (M + H)⁺. 1H NMR (400 MHz, DMSO) δ 8.56 (s, 2H), 7.69 (d, J = 8.7 Hz, 1H), 7.09 (t, J = 9.1 Hz, 1H), 6.95 (d, J = 8.7 Hz, 1H), 6.88 (dt, J = 9.0, 3.6 Hz, 1H), 6.74 (dd, J = 6.0, 3.1 Hz, 1H), 3.72 (s, 3H), 3.52 (s, 4H). |
| 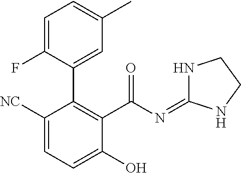 [SMILES: Cc1ccc(F)c(-c2c(C#N)ccc(O)c2C(=O)N=C2NCCN2)c1] | 6-cyano-2′-fluoro-3-hydroxy-N-(imidazolidin-2- ylidene)-5′-methyl-[1,1′-biphenyl]-2- carboxamide LC-MS: m/z 339 (M + H)⁺. 1H NMR (400 MHz, DMSO) δ 8.56 (s, 2H), 7.70 (d, J = 8.7 Hz, 1H), 7.17-7.12 (m, 1H), 7.09-7.02 (m, 1H), 7.01-6.93 (m, 2H), 3.53 (s, 4H), 2.28 (s, 3H). |
| 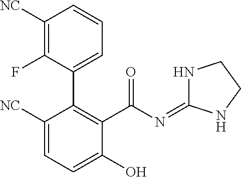 [SMILES: N#Cc1cccc(-c2c(C#N)ccc(O)c2C(=O)N=C2NCCN2)c1F] | 3′,6-dicyano-2′-fluoro-3-hydroxy-N- (imidazolidin-2-ylidene)-[1,1′-biphenyl]-2- carboxamide LC-MS: m/z 345 (M + H)⁺. 1H NMR (400 MHz, DMSO) δ: 8.52 (s, 2H), 7.97-7.89 (m, 1H), 7.76 (d, J = 8.7 Hz, 1H), 7.65 (td, J = 7.5, 1.6 Hz, 1H), 7.44 (t, J = 7.7 Hz, 1H), 7.01 (d, J = 8.7 Hz, 1H), 3.53 (s, 4H). |
| 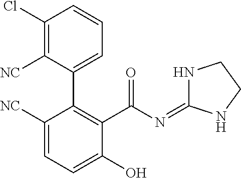 [SMILES: N#Cc1ccc(O)c(C(=O)N=C2NCCN2)c1-c1cccc(Cl)c1C#N] | 3′-chloro-2′,6-dicyano-3-hydroxy-N- (imidazolidin-2-ylidene)-[1,1′-biphenyl]-2- carboxamide LC-MS: m/z 366 (M + H)⁺. 1H NMR (400 MHz, DMSO) δ: 8.60 (s, 2H), 7.79 (d, J = 8.7 Hz, 1H), 7.76-7.70 (m, 2H), 7.39 (dd, J = 6.6, 2.2 Hz, 1H), 7.04 (d, J = 8.7 Hz, 1H), 3.54 (s, 4H). |
| 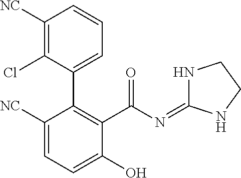 [SMILES: N#Cc1cccc(-c2c(C#N)ccc(O)c2C(=O)N=C2NCCN2)c1Cl] | 2′-chloro-3′,6-dicyano-3-hydroxy-N- (imidazolidin-2-ylidene)-[1,1′-biphenyl]-2- carboxamide LC-MS: m/z 366 (M + H)⁺. 1H NMR (400 MHz, DMSO) δ: 7.96 (dd, J = 7.3, 2.1 Hz, 1H), 7.76 (d, J = 8.7 Hz, 1H), 7.63- 7.53 (m, 2H), 7.00 (d, J = 8.7 Hz, 1H), 3.52 (s, 4H). |
| 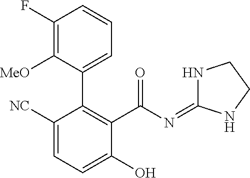 [SMILES: COc1c(F)cccc1-c1c(C#N)ccc(O)c1C(=O)N=C1NCCN1] | 6-cyano-3′-fluoro-3-hydroxy-N-(imidazolidin-2- ylidene)-2′-methoxy-[1,1′-biphenyl]-2- carboxamide LC-MS: m/z 355 (M + H)⁺. 1H NMR (400 MHz, DMSO) δ 7.68 (d, J = 8.7 Hz, 1H), 7.21 (ddd, J = 11.9, 8.3, 1.5 Hz, 1H), 7.08 (td, J = 8.0, 5.1 Hz, 1H), 6.94 (d, J = 8.7 Hz, 1H), 6.90-6.82 (m, 1H), 3.64 (s, 3H), 3.53 (s, 4H). |
| 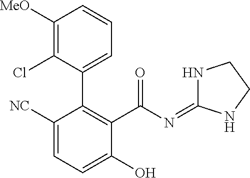 [SMILES: COc1cccc(-c2c(C#N)ccc(O)c2C(=O)N=C2NCCN2)c1Cl] | 2′-chloro-6-cyano-3-hydroxy-N-(imidazolidin-2- ylidene)-3′-methoxy-[1,1′-biphenyl]-2- carboxamide LC-MS: m/z 371 (M + H)⁺. 1H NMR (400 MHz, DMSO) δ: 8.55 (s, 2H), 7.69 (d, J = 8.7 Hz, 1H), 7.29 (t, J = 8.0 Hz, 1H), 7.08 (dd, J = 8.4, 1.2 Hz, 1H), 6.95 (d, J = 8.7 Hz, 1H), 6.77 (dd, J = 7.6, 1.3 Hz, 1H), 3.88 (s, 3H), 3.52 (s, 4H). |
| 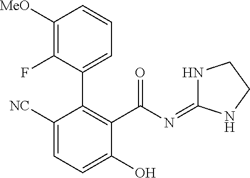 [SMILES: COc1cccc(-c2c(C#N)ccc(O)c2C(=O)N=C2NCCN2)c1F] | 6-cyano-2′-fluoro-3-hydroxy-N-(imidazolidin-2- ylidene)-3′-methoxy-[1,1′-biphenyl]-2- carboxamide LC-MS: m/z 355 (M + H)⁺. 1H NMR (400 MHz, DMSO) δ 8.55 (br s, 2H), 7.70 (d, J = 8.7 Hz, 1H), 7.15-7.08 (m, 2H), 6.96 (d, J = 8.7 Hz, 1H), 6.76-6.69 (m, 1H), 3.86 (s, 3H), 3.53 (s, 4H). |
| 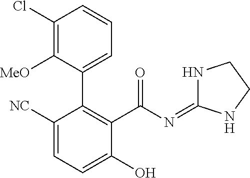 [SMILES: COc1c(Cl)cccc1-c1c(C#N)ccc(O)c1C(=O)N=C1NCCN1] | 3′-chloro-6-cyano-3-hydroxy-N-(imidazolidin-2- ylidene)-2′-methoxy-[1,1′-biphenyl]-2- carboxamide LC-MS: m/z 371 (M + H)⁺. 1H NMR (400 MHz, DMSO) δ 8.27 (br s, 2H), 7.70 (d, J = 8.7 Hz, 1H), 7.43 (dd, J = 8.0, 1.6 Hz, 1H), 7.13 (t, J = 7.8 Hz, 1H), 7.04 (dd, J = 7.6, 1.6 Hz, 1H), 6.96 (d, J = 8.7 Hz, 1H), 3.52 (s, 4H), 3.50 (s, 3H). |
| 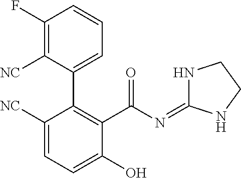 [SMILES: N#Cc1ccc(O)c(C(=O)N=C2NCCN2)c1-c1cccc(F)c1C#N] | 2′,6-dicyano-3′-fluoro-3-hydroxy-N- (imidazolidin-2-ylidene)-[1,1′-biphenyl]-2- carboxamide LC-MS: m/z 350 (M + H)⁺. 1H NMR (400 MHz, DMSO) δ: 8.59 (s, 2H), 7.84-7.76 (m, 2H), 7.52 (t, J = 9.0 Hz, 1H), 7.27 (d, J = 8.7 Hz, 1H), 7.05 (d, J = 8.7 Hz, 1H), 3.54 (s, 4H). |
| 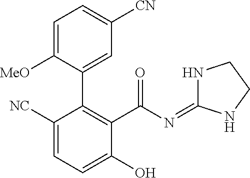 [SMILES: COc1ccc(C#N)cc1-c1c(C#N)ccc(O)c1C(=O)N=C1NCCN1] | 5′,6-dicyano-3-hydroxy-N-(imidazolidin-2- ylidene)-2′-methoxy-[1,1′-biphenyl]-2- carboxamide LC-MS: m/z 362 (M + H)⁺. 1H NMR (400 MHz, DMSO) δ 8.52 (s, 2H), 7.81 (dd, J = 8.6, 2.1 Hz, 1H), 7.68 (d, J-8.7 Hz, 1H), 7.50 (d, J = 2.1 Hz, 1H), 7.22 (d, J = 8.7 Hz, 1H), 6.94 (d, J = 8.7 Hz, 1H), 3.75 (s, 3H), 3.52 (s, 4H). |
| 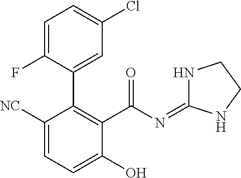 [SMILES: N#Cc1ccc(O)c(C(=O)N=C2NCCN2)c1-c1cc(Cl)ccc1F] | 5′-chloro-6-cyano-2′-fluoro-3-hydroxy-N- (imidazolidin-2-ylidene)-[1,1′-biphenyl]-2- carboxamide LC-MS: m/z 359 (M + H)⁺. 1H NMR (400 MHz, DMSO) δ 8.58 (s, 2H), 7.72 (d, J = 8.7 Hz, 1H), 7.42 (ddd, J = 8.7, 4.4, 2.7 Hz, 1H), 7.33 (dd, J = 6.3, 2.7 Hz, 1H), 7.26 (t, J = 9.0 Hz, 1H), 6.98 (d, J = 8.7 Hz, 1H), 3.53 (s, 4H). |
| 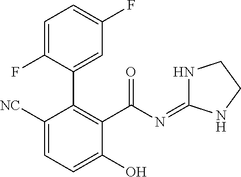 [SMILES: N#Cc1ccc(O)c(C(=O)N=C2NCCN2)c1-c1cc(F)ccc1F] | 6-cyano-2′,5′-difluoro-3-hydroxy-N- (imidazolidin-2-ylidene)-[1,1′-biphenyl]-2- carboxamide LC-MS: m/z 343 (M + H)⁺. 1H NMR (400 MHz, DMSO) δ 8.59 (s, 2H), 7.73 (d, J = 8.7 Hz, 1H), 7.29-7.12 (m, 3H), 6.99 (d, J = 8.7 Hz, 1H), 3.54 (s, 4H). |
| 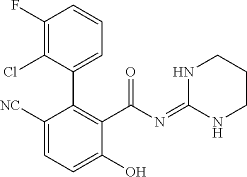 [SMILES: N#Cc1ccc(O)c(C(=O)N=C2NCCCN2)c1-c1cccc(F)c1Cl] | 2′-chloro-6-cyano-3′-fluoro-3-hydroxy-N- (tetrahydropyrimidin-2(1H)-ylidene)-[1,1′- biphenyl]-2-carboxamide LC-MS: m/z 373 (M + H)⁺. 1H NMR (400 MHz, DMSO) δ 8.91 (s, 2H), 7.67 (d, J = 8.7 Hz, 1H), 7.40-7.31 (m, 2H), 7.07-7.02 (m, 1H), 6.91 (d, J = 8.7 Hz, 1H), 3.25 (t, J = 5.7 Hz, 4H), 1.82-1.74 (m, 2H). |
| 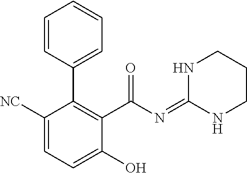 [SMILES: N#Cc1ccc(O)c(C(=O)N=C2NCCCN2)c1-c1ccccc1] | 6-cyano-3-hydroxy-N-(tetrahydropyrimidin- 2(1H)-ylidene)-[1,1′-biphenyl]-2-carboxamide LC-MS: m/z 321 (M + H)⁺. 1H NMR (400 MHz, DMSO) δ 8.90 (s, 2H), 7.61 (d, J = 8.7 Hz, 1H), 7.38-7.27 (m, 3H), 7.16 (d, J = 6.7 Hz, 2H), 6.87 (d, J = 8.7 Hz, 1H), 3.24 (t, J = 5.7 Hz, 4H), 1.84-1.73 (m, 2H). |
| 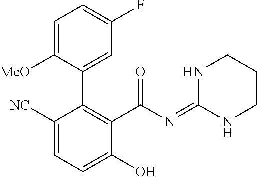 [SMILES: COc1ccc(F)cc1-c1c(C#N)ccc(O)c1C(=O)N=C1NCCCN1] | 6-cyano-5′-fluoro-3-hydroxy-2′-methoxy-N- (tetrahydropyrimidin-2(1H)-ylidene)-[1,1′- biphenyl]-2-carboxamide LC-MS: m/z 369 (M + H)⁺. 1H NMR (400 MHz, DMSO) δ 8.81 (br s, 2H), 7.60 (d, J = 8.7 Hz, 1H), 7.09 (td, J = 8.7, 3.1 Hz, 1H), 7.00 (dd, J = 9.1, 4.6 Hz, 1H), 6.86 (dd, J = 8.8, 1.9 Hz, 2H), 3.64 (s, 3H), 3.26 (t, J = 5.7 Hz, 4H), 1.78 (dd, J = 10.6, 5.7 Hz, 2H). |
| 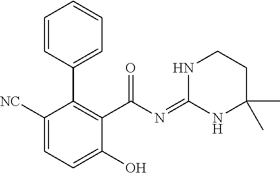 [SMILES: CC1(C)CCN/C(=N\C(=O)c2c(O)ccc(C#N)c2-c2ccccc2)N1] | (E)-6-cyano-N-(4,4- dimethyltetrahydropyrimidin-2(1H)-ylidene)-3- hydroxy-[1,1′-biphenyl]-2-carboxamide LC-MS: m/z 349 (M + H)⁺. 1H NMR (400 MHz, DMSO) δ 9.27 (s, 1H), 8.72 (s, 1H), 7.61 (d, J = 8.6 Hz, 1H), 7.38-7.29 (m, 3H), 7.15 (d, J = 6.5 Hz, 2H), 6.87 (d, J = 8.7 Hz, 1H), 2.50-2.47 (m, 2H), 1.67 (t, J = 5.9 Hz, 2H), 1.22 (s, 6H). |
| 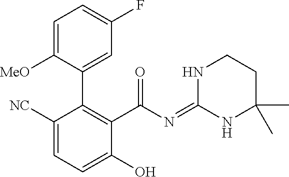 [SMILES: COc1ccc(F)cc1-c1c(C#N)ccc(O)c1C(=O)/N=C1\NCCC(C)(C)N1] | (E)-6-cyano-N-(4,4- dimethyltetrahydropyrimidin-2(1H)-ylidene)-5′- fluoro-3-hydroxy-2′-methoxy-[1,1′-biphenyl]-2- carboxamide LC-MS: m/z 397 (M + H)⁺. 1H NMR (400 MHz, DMSO) δ 9.29 (s, 1H), 8.71 (s, 1H), 7.60 (d, J = 8.8 Hz, 1H), 7.09 (td, J = 8.8, 3.2 Hz, 1H), 7.00 (dd, J = 9.2, 4.8 Hz, 1H), 6.85 (dd, J = 8.4, 2.0 Hz, 2H), 3.64 (s, 3H), 3.31- 3.27 (m, 2H), 1.67 (t, J = 6.0 Hz, 2H), 1.22 (s, 6H). |
| 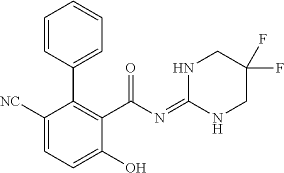 [SMILES: N#Cc1ccc(O)c(C(=O)N=C2NCC(F)(F)CN2)c1-c1ccccc1] | 6-cyano-N-(5,5-difluorotetrahydropyrimidin- 2(1H)-ylidene)-3-hydroxy-[1,1′-biphenyl]-2- carboxamide LC-MS: m/z 357 (M + H)⁺. 1H NMR (400 MHz, DMSO) δ 9.29 (s, 2H), 7.69 (d, J = 8.7 Hz, 1H), 7.39-7.29 (m, 3H), 7.21-7.16 (m, 2H), 6.96 (d, J = 8.6 Hz, 1H), 3.68 (t, J = 12.4 Hz, 4H). |
| 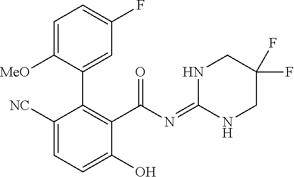 [SMILES: COc1ccc(F)cc1-c1c(C#N)ccc(O)c1C(=O)N=C1NCC(F)(F)CN1] | 6-cyano-N-(5,5-difluorotetrahydropyrimidin- 2(1H)-ylidene)-5′-fluoro-3-hydroxy-2′-methoxy- [1,1′-biphenyl]-2-carboxamide LC-MS: m/z 405 (M + H)⁺. 1H NMR (400 MHz, DMSO) δ 9.31 (s, 2H), 7.68 (d, J = 8.6 Hz, 1H), 7.11 (td, J = 8.7, 3.1 Hz, 1H), 7.02 (dd, J = 9.1, 4.6 Hz, 1H), 6.94 (d, J = 8.6 Hz, 1H), 6.90 (dd, J = 8.9, 3.1 Hz, 1H), 3.74- 3.66 (m, 4H), 3.65 (s, 3H). |
| 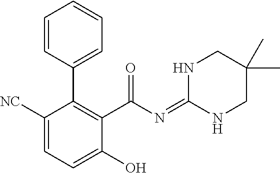 [SMILES: CC1(C)CNC(=NC(=O)c2c(O)ccc(C#N)c2-c2ccccc2)NC1] | 6-cyano-N-(5,5-dimethyltetrahydropyrimidin- 2(1H)-ylidene)-3-hydroxy-[1,1′-biphenyl]-2- carboxamide LC-MS: m/z 349 (M + H)⁺. 1H NMR (400 MHz, DMSO) δ 8.96 (br s, 2H), 7.61 (d, J = 8.7 Hz, 1H), 7.44-7.25 (m, 3H), 7.22-7.10 (m, 2H), 6.88 (d, J = 8.7 Hz, 1H), 2.95 (s, 4H), 0.94 (s, 6H). |
| 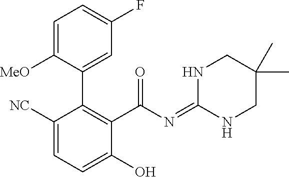 [SMILES: COc1ccc(F)cc1-c1c(C#N)ccc(O)c1C(=O)N=C1NCC(C)(C)CN1] | 6-cyano-N-(5,5-dimethyltetrahydropyrimidin- 2(1H)-ylidene)-5′-fluoro-3-hydroxy-2′-methoxy- [1,1′-biphenyl]-2-carboxamide LC-MS: m/z 397 (M + H)⁺. 1H NMR (400 MHz, DMSO) δ 8.95 (s, 2H), 7.60 (d, J = 8.7 Hz, 1H), 7.08 (td, J = 8.7, 3.1 Hz, 1H), 6.99 (dd, J = 9.1, 4.6 Hz, 1H), 6.89-6.81 (m, 2H), 3.63 (s, 3H), 2.95 (s, 4H), 0.93 (s, 6H). |
| 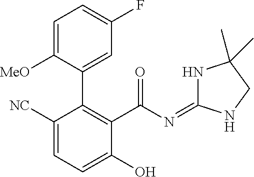 [SMILES: COc1ccc(F)cc1-c1c(C#N)ccc(O)c1C(=O)/N=C1/NCC(C)(C)N1] | (Z)-6-cyano-N-(4,4-dimethylimidazolidin-2- ylidene)-5′-fluoro-3-hydroxy-2′-methoxy-[1,1′- biphenyl]-2-carboxamide LC-MS: m/z 383 (M + H)⁺. 1H NMR (400 MHz, DMSO) δ 8.56 (br s, 2H), 7.70 (d, J = 8.8 Hz, 1H), 7.15 (td, J = 9.2, 3.2 Hz, 1H), 7.06 (dd, J = 9.2, 4.8 Hz, 1H), 7.00-6.90 (m, 2H), 3.69 (s, 3H), 3.33 (s, 2H), 1.31 (s, 6H). |
| 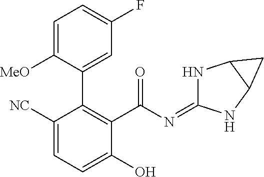 [SMILES: COc1ccc(F)cc1-c1c(C#N)ccc(O)c1C(=O)N=C1NC2CC2N1] | N-(2,4-diazabicyclo[3.1.0]hexan-3-ylidene)-6- cyano-5′-fluoro-3-hydroxy-2′-methoxy-[1,1′- biphenyl]-2-carboxamide LC-MS: m/z 367 (M + H)⁺. 1H NMR (400 MHz, DMSO) δ 9.26 (s, 2H), 7.65 (d, J = 8.6 Hz, 1H), 7.12-7.06 (m, 1H), 7.00 (dd, J = 9.1, 4.6 Hz, 1H), 6.93-6.84 (m, 2H), 3.62 (s, 3H), 3.46 (dd, J = 6.1, 2.4 Hz, 2H), 0.75-0.71 (m, 1H), 0.08-0.04 (m, 1H). |
| 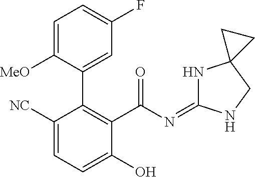 [SMILES: COc1ccc(F)cc1-c1c(C#N)ccc(O)c1C(=O)/N=C1/NCC2(CC2)N1] | (Z)-6-cyano-5′-fluoro-3-hydroxy-2′-methoxy-N- (4,6-diazaspiro[2.4]heptan-5-ylidene)-[1,1′- biphenyl]-2-carboxamide LC-MS: m/z 381 (M + H)⁺. 1H NMR (400 MHz, DMSO) δ 8.67 (s, 1H), 8.57 (s, 1H), 7.66 (d, J = 8.8 Hz, 1H), 7.09 (td, J = 8.8, 3.2 Hz, 1H), 7.00 (dd, J = 8.8, 4.4 Hz, 1H), 6.95-6.86 (m, 2H), 3.64 (s, 3H), 3.56 (s, 2H), 0.95 (q, J = 5.6 Hz, 2H), 0.69 (q, J = 5.6 Hz, 2H). |
| 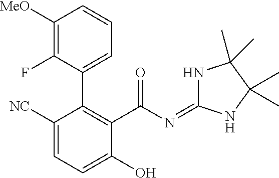 [SMILES: COc1cccc(-c2c(C#N)ccc(O)c2C(=O)N=C2NC(C)(C)C(C)(C)N2)c1F] | 6-cyano-2′-fluoro-3-hydroxy-3′-methoxy-N- (4,4,5,5-tetramethylimidazolidin-2-ylidene)- [1,1′-biphenyl]-2-carboxamide LC-MS: m/z 411 (M + H)⁺. 1H NMR (400 MHz, DMSO) δ 8.59 (s, 2H), 7.69 (d, J = 8.7 Hz, 1H), 7.15-7.08 (m, 2H), 6.95 (d, J = 8.7 Hz, 1H), 6.76-6.68 (m, 1H), 3.86 (s, 3H), 1.15 (s, 12H). |
| 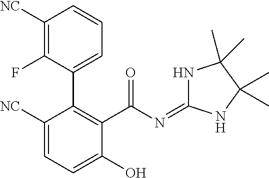 [SMILES: CC1(C)NC(=NC(=O)c2c(O)ccc(C#N)c2-c2cccc(C#N)c2F)NC1(C)C] | 3′,6-dicyano-2′-fluoro-3-hydroxy-N-(4,4,5,5- tetramethylimidazolidin-2-ylidene)-[1,1′- biphenyl]-2-carboxamide LC-MS: m/z 406 (M + H)⁺. 1H NMR (400 MHz, DMSO) δ 8.64 (s, 2H), 7.95-7.91 (m, 1H), 7.76 (d, J = 8.7 Hz, 1H), 7.65 (td, J = 7.6, 1.7 Hz, 1H), 7.46 (t, J = 7.7 Hz, 1H), 7.02 (d, J = 8.7 Hz, 1H), 1.15 (s, 12H). |
| 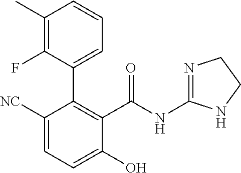 [SMILES: Cc1cccc(-c2c(C#N)ccc(O)c2C(=O)NC2=NCCN2)c1F] | 6-cyano-N-(4,5-dihydro-1H-imidazol-2-yl)-2′- fluoro-3-hydroxy-3′-methyl-[1,1′-biphenyl]-2- carboxamide LC-MS: m/z 339 (M + H)⁺. 1H NMR (400 MHz, DMSO-d6) δ 8.50 (br s, 2H), 7.68 (d, J = 8.7 Hz, 1H), 7.23 (t, J = 7.0 Hz, 1H), 7.07 (t, J = 7.5 Hz, 1H), 6.99 (t, J = 6.7 Hz, 1H), 6.94 (d, J = 8.7 Hz, 1H), 3.52 (s, 4H), 2.24 (s, 3H). |
| 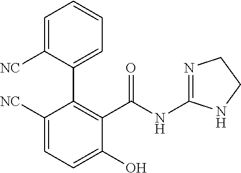 [SMILES: N#Cc1ccccc1-c1c(C#N)ccc(O)c1C(=O)NC1=NCCN1] | 2′,6-dicyano-N-(4,5-dihydro-1H-imidazol-2-yl)- 3-hydroxy-[1,1′-biphenyl]-2-carboxamide LC-MS: m/z 332 (M + H)⁺. 1H NMR (400 MHz, DMSO-d6) δ 8.50 (br s, 2H), 7.82-7.76 (m, 1H), 7.69 (d, J = 8.7 Hz, 1H), 7.64 (td, J = 7.7, 1.2 Hz, 1H), 7.46 (td, J = 7.7, 1.1 Hz, 1H), 7.31 (d, J = 7.4 Hz, 1H), 6.95 (d, J = 8.7 Hz, 1H), 3.46 (s, 4H). |
| 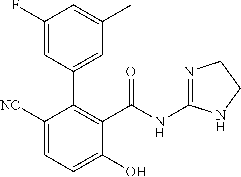 [SMILES: Cc1cc(F)cc(-c2c(C#N)ccc(O)c2C(=O)NC2=NCCN2)c1] | 6-cyano-N-(4,5-dihydro-1H-imidazol-2-yl)-3′- fluoro-3-hydroxy-5′-methyl-[1,1′-biphenyl]-2- carboxamide LC-MS: m/z 339 (M + H)⁺. 1H NMR (400 MHz, DMSO-d6) δ 7.62 (d, J = 8.7 Hz, 1H), 6.96 (d, J = 10.1 Hz, 1H), 6.89 (d, J = 8.7 Hz, 1H), 6.81 (s, 1H), 6.79 (d, J = 1.7 Hz, 1H), 3.51 (s, 4H), 2.33 (s, 3H). |
| 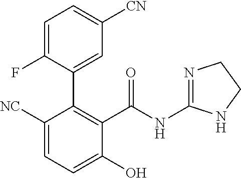 [SMILES: N#Cc1ccc(F)c(-c2c(C#N)ccc(O)c2C(=O)NC2=NCCN2)c1] | 5′,6-dicyano-N-(4,5-dihydro-1H-imidazol-2-yl)- 2′-fluoro-3-hydroxy-[1,1′-biphenyl]-2- carboxamide LC-MS: m/z 350 (M + H)⁺. 1 HNMR (400 MHz, DMSO) δ 8.55 (brs, 2H), 7.99-7.91 (m, 1H), 7.88 (dd, J = 6.4, 2.0 Hz, 1H), 7.76 (d, J = 8.8 Hz, 1H), 7.49 (t, J = 9.2 Hz, 1H), 7.02 (d, J = 8.8 Hz, 1H), 3.54 (s, 4H). |
| 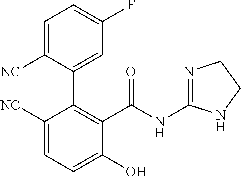 [SMILES: N#Cc1ccc(F)cc1-c1c(C#N)ccc(O)c1C(=O)NC1=NCCN1] | 2′,6-dicyano-N-(4,5-dihydro-1H-imidazol-2-yl)- 5′-fluoro-3-hydroxy-[1,1′-biphenyl]-2- carboxamide LC-MS: m/z 350 (M + H)⁺. 1 HNMR (400 MHz, DMSO) δ 8.60 (brs, 2H), 8.03-7.94 (m, 1H), 7.78 (d, J = 8.7 0 Hz, 1H), 7.43-7.39 (m, 2H), 7.03 (d, J = 8.7 0 Hz, 1H), 3.54 (s, 4H). |

### General Procedure 10

Combination of the general procedures 5 and 9 described above.

The compounds in the table below were prepared from the appropriate starting materials described previously or commercially available using above general procedure 10

| Structure | Name |
|---|---|
| 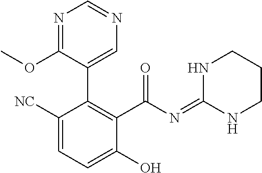 [SMILES: COc1ncncc1-c1c(C#N)ccc(O)c1C(=O)N=C1NCCCN1] | 3-cyano-6-hydroxy-2-(4-methoxypyrimidin-5- yl)-N-(tetrahydropyrimidin-2(1H)- ylidene)benzamide LC-MS: m/z 353 (M + H)⁺. 1H NMR (400 MHz, DMSO) δ 8.92 (s, 2H), 8.77 (s, 1H), 8.26 (s, 1H), 7.68 (d, J = 8.8 Hz, 1H), 6.92 (d, J = 8.4 Hz, 1H), 3.86 (s, 3H), 3.26 (t, J = 6.0 Hz, 4H), 1.83-1.73 (m, 2H). |
| 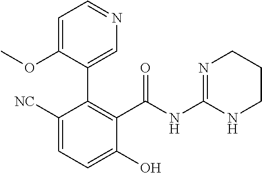 [SMILES: COc1ccncc1-c1c(C#N)ccc(O)c1C(=O)NC1=NCCCN1] | 3-cyano-6-hydroxy-2-(4-methoxypyridin-3-yl)- N-(1,4,5,6-tetrahydropyrimidin-2-yl)benzamide LC-MS: m/z 352 (M + H)⁺. 1H NMR (400 MHz, DMSO) δ 8.89 (s, 2H), 8.40 (d, J = 6.0 Hz, 1H), 8.02 (s, 1H), 7.63 (d, J = 8.4 Hz, 1H), 7.07 (d, J = 6.0 Hz, 1H), 6.88 (d, J = 8.8 Hz, 1H), 3.75 (s, 3H), 3.25 (t, J = 5.6 Hz, 4H), 1.83-1.74 (m, 2H). |
| 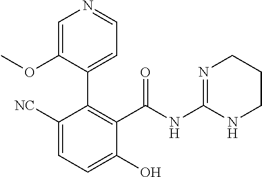 [SMILES: COc1cnccc1-c1c(C#N)ccc(O)c1C(=O)NC1=NCCCN1] | 3-cyano-6-hydroxy-2-(3-methoxypyridin-4-yl)- N-(1,4,5,6-tetrahydropyrimidin-2-yl)benzamide LC-MS: m/z 352 (M + H)⁺. 1H NMR (400 MHz, DMSO) δ 8.89 (s, 2H), 8.37 (s, 1H), 8.19 (d, J = 4.7 Hz, 1H), 7.63 (d, J = 8.7 Hz, 1H), 7.04 (d, J = 4.7 Hz, 1H), 6.88 (d, J = 8.7 Hz, 1H), 3.78 (s, 3H), 3.24 (t, J = 5.7 Hz, 4H), 1.80-1.75 (m, 2H). |
| 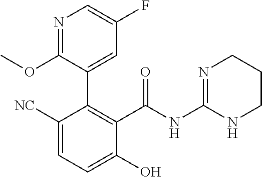 [SMILES: COc1ncc(F)cc1-c1c(C#N)ccc(O)c1C(=O)NC1=NCCCN1] | 3-cyano-2-(5-fluoro-2-methoxypyridin-3-yl)-6- hydroxy-N-(1,4,5,6-tetrahydropyrimidin-2- yl)benzamide LC-MS: m/z 370 (M + H)⁺. 1H NMR (400 MHz, DMSO) δ 8.90 (s, 2H), 8.09 (d, J = 3.2 Hz, 1H), 7.64 (d, J = 8.8 Hz, 1H), 7.51 (dd, J = 8.4, 2.8 Hz, 1H), 6.88 (d, J = 8.8 Hz, 1H), 3.74 (s, 3H), 3.25 (t, J = 5.6 Hz, 4H), 1.85-1.73 (m, 2H). |
| 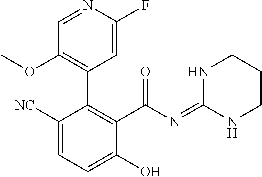 [SMILES: COc1cnc(F)cc1-c1c(C#N)ccc(O)c1C(=O)N=C1NCCCN1] | 3-cyano-2-(2-fluoro-5-methoxypyridin-4-yl)-6- hydroxy-N-(tetrahydropyrimidin-2(1H)- ylidene)benzamide LC-MS: m/z 370 (M + H)⁺. 1H NMR (400 MHz, DMSO) δ 8.90 (s, 2H), 7.92 (d, J = 1.4 Hz, 1H), 7.66 (d, J = 8.7 Hz, 1H), 6.97 (d, J = 2.9 Hz, 1H), 6.91 (d, J = 8.7 Hz, 1H), 3.76 (s, 3H), 3.26 (t, J = 5.7 Hz, 4H), 1.84-1.74 (m, 2H). |
| 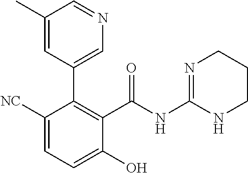 [SMILES: Cc1cncc(-c2c(C#N)ccc(O)c2C(=O)NC2=NCCCN2)c1] | 3-cyano-6-hydroxy-2-(5-methylpyridin-3-yl)-N- (1,4,5,6-tetrahydropyrimidin-2-yl)benzamide LC-MS: m/z 336 (M + H)⁺. 1H NMR (400 MHz, DMSO) δ 8.65 (br s, 2H), 8.40 (d, J = 1.6 Hz, 1H), 8.19 (d, J = 2.0 Hz, 1H), 7.68 (d, J = 8.8 Hz, 1H), 7.48 (s, 1H), 6.93 (d, J = 8.4 Hz, 1H), 3.30 (t, J = 5.6 Hz, 4H), 2.38 (s, 3H), 1.89-1.76 (m, 2H). |
| 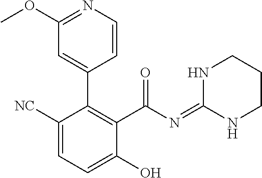 [SMILES: COc1cc(-c2c(C#N)ccc(O)c2C(=O)N=C2NCCCN2)ccn1] | 3-cyano-6-hydroxy-2-(2-methoxypyridin-4-yl)- N-(tetrahydropyrimidin-2(1H)- ylidene)benzamide LC-MS: m/z 352 (M + H)⁺. 1H NMR (400 MHz, DMSO) δ 8.93 (s, 2H), 8.12 (d, J = 5.3 Hz, 1H), 7.63 (d, J = 8.7 Hz, 1H), 6.89 (d, J = 8.7 Hz, 1H), 6.79 (dd, J = 5.2, 1.4 Hz, 1H), 6.61 (d, J = 0.5 Hz, 1H), 3.88 (s, 3H), 3.25 (t, J = 5.7 Hz, 4H), 1.84-1.72 (m, 2H). |
| 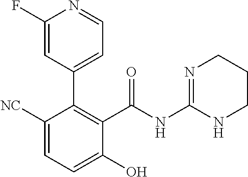 [SMILES: N#Cc1ccc(O)c(C(=O)NC2=NCCCN2)c1-c1ccnc(F)c1] | 3-cyano-2-(2-fluoropyridin-4-yl)-6-hydroxy-N- (1,4,5,6-tetrahydropyrimidin-2-yl)benzamide LC-MS: m/z 340 (M + H)⁺. 1H NMR (400 MHz, DMSO) δ 8.89 (br s, 2H), 8.20 (d, J = 5.2 Hz, 1H), 7.64 (d, J = 8.8 Hz, 1H), 7.23-7.13 (m, 1H), 7.07 (s, 1H), 6.89 (d, J = 8.8 Hz, 1H), 3.24 (t, J = 5.6 Hz, 4H), 1.84- 1.70 (m, 2H). |
| 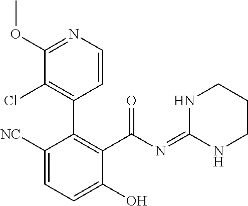 [SMILES: COc1nccc(-c2c(C#N)ccc(O)c2C(=O)N=C2NCCCN2)c1Cl] | 2-(3-chloro-2-methoxypyridin-4-yl)-3-cyano-6- hydroxy-N-(tetrahydropyrimidin-2(1H)- ylidene)benzamide LC-MS: m/z 386 (M + H)⁺. 1H NMR (400 MHz, DMSO-d6) δ: 8.92 (s, 2H), 8.10 (d, J = 5.1 Hz, 1H), 7.71 (d, J = 8.7 Hz, 1H), 6.94 (d, J = 8.5 Hz, 1H), 6.89 (d, J = 5.1 Hz, 1H), 3.97 (s, 3H), 3.26 (t, J = 5.7 Hz, 4H), 1.83-1.78 (m, 2H). |
| 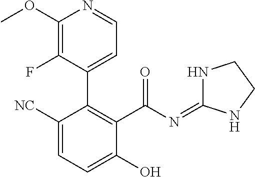 [SMILES: COc1nccc(-c2c(C#N)ccc(O)c2C(=O)N=C2NCCN2)c1F] | 3-cyano-2-(3-fluoro-2-methoxypyridin-4-yl)-6- hydroxy-N-(imidazolidin-2-ylidene)benzamide LC-MS: m/z 356 (M + H)⁺. 1H NMR (400 MHz, DMSO) δ 8.63 (s, 2H), 7.96 (d, J = 5.1 Hz, 1H), 7.76 (d, J = 8.7 Hz, 1H), 7.01 (d, J = 8.7 Hz, 1H), 6.89 (t, J = 4.8 Hz, 1H), 3.97 (s, 3H), 3.54 (s, 4H). |
| 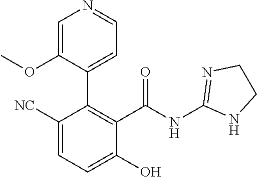 [SMILES: COc1cnccc1-c1c(C#N)ccc(O)c1C(=O)NC1=NCCN1] | 3-cyano-N-(4,5-dihydro-1H-imidazol-2-yl)-6- hydroxy-2-(3-methoxypyridin-4-yl)benzamide LC-MS: m/z 338 (M + H)⁺. 1H NMR (400 MHz, DMSO) δ 8.54 (s, 2H), 8.38 (s, 1H), 8.20 (d, J = 4.7 Hz, 1H), 7.69 (d, J = 8.7 Hz, 1H), 7.07 (d, J = 4.7 Hz, 1H), 6.94 (d, J = 8.7 Hz, 1H), 3.77 (s, 3H), 3.52 (s, 4H). |
| 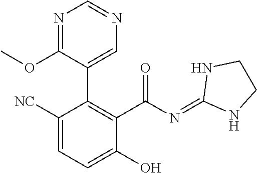 [SMILES: COc1ncncc1-c1c(C#N)ccc(O)c1C(=O)N=C1NCCN1] | 3-cyano-6-hydroxy-N-(imidazolidin-2-ylidene)- 2-(4-methoxypyrimidin-5-yl)benzamide LC-MS: m/z 339 (M + H)⁺. 1H NMR (400 MHz, DMSO) δ 8.78 (s, 1H), 8.54 (br s, 2H), 8.28 (s, 1H), 7.73 (d, J = 8. 8 Hz, 1H), 6.97 (d, J = 8.8 Hz, 1H), 3.85 (s, 3H), 3.53 (s, 4H). |
| 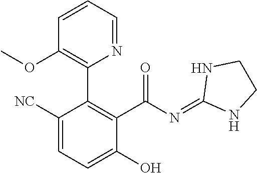 [SMILES: COc1cccnc1-c1c(C#N)ccc(O)c1C(=O)N=C1NCCN1] | 3-cyano-6-hydroxy-N-(imidazolidin-2-ylidene)- 2-(3-methoxypyridin-2-yl)benzamide LC-MS: m/z 338 (M + H)⁺. 1H NMR (400 MHz, DMSO) δ 8.45 (s, 2H), 8.10 (dd, J = 4.7, 1.2 Hz, 1H), 7.67 (d, J = 8.6 Hz, 1H), 7.42 (dd, J = 8.4, 1.2 Hz, 1H), 7.31 (dd, J = 8.3, 4.7 Hz, 1H), 6.94 (d, J = 8.6 Hz, 1H), 3.70 (s, 3H), 3.52 (s, 4H). |
| 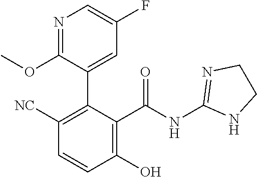 [SMILES: COc1ncc(F)cc1-c1c(C#N)ccc(O)c1C(=O)NC1=NCCN1] | 3-cyano-N-(4,5-dihydro-1H-imidazol-2-yl)-2- (5-fluoro-2-methoxypyridin-3-yl)-6- hydroxybenzamide LC-MS: m/z 356 (M + H)⁺. 1H NMR (400 MHz, DMSO) δ 8.27 (br s, 1H), 8.09 (d, J = 2.8 Hz, 1H), 7.65 (d, J = 8.8 Hz, 1H), 7.53 (dd, J = 8.4, 3.2 Hz, 1H), 6.90 (d, J = 8.8 Hz, 1H), 3.74 (s, 3H), 3.50 (s, 4H). |
| 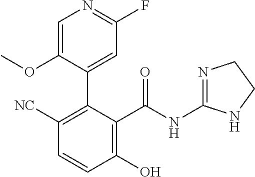 [SMILES: COc1cnc(F)cc1-c1c(C#N)ccc(O)c1C(=O)NC1=NCCN1] | 3-cyano-N-(4,5-dihydro-1H-imidazol-2-yl)-2- (2-fluoro-5-methoxypyridin-4-yl)-6- hydroxybenzamide LC-MS: m/z 356 (M + H)⁺. 1H NMR (400 MHz, DMSO) δ 8.48 (br s, 2H), 7.92 (d, J = 1.5 Hz, 1H), 7.69 (d, J = 8.7 Hz, 1H), 6.98 (d, J = 2.9 Hz, 1H), 6.93 (d, J = 8.7 Hz, 1H), 3.75 (s, 3H), 3.51 (s, 4H). |
| 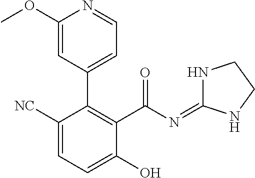 [SMILES: COc1cc(-c2c(C#N)ccc(O)c2C(=O)N=C2NCCN2)ccn1] | 3-cyano-6-hydroxy-N-(imidazolidin-2-ylidene)- 2-(2-methoxypyridin-4-yl)benzamide LC-MS: m/z 338 (M + H)⁺. 1H NMR (400 MHz, DMSO) δ 8.58 (s, 2H), 8.13 (d, J = 5.2 Hz, 1H), 7.69 (d, J = 8.7 Hz, 1H), 6.95 (d, J = 8.7 Hz, 1H), 6.80 (dd, J = 5.2, 1.4 Hz, 1H), 6.63 (s, 1H), 3.87 (s, 3H), 3.52 (s, 4H). |
| 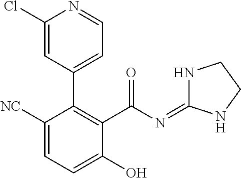 [SMILES: N#Cc1ccc(O)c(C(=O)N=C2NCCN2)c1-c1ccnc(Cl)c1] | 2-(2-chloropyridin-4-yl)-3-cyano-6-hydroxy-N- (imidazolidin-2-ylidene)benzamide LC-MS: m/z 342 (M + H)⁺. 1H NMR (400 MHz, DMSO) δ 8.56 (br s, 2H), 8.40 (d, J = 5.0 Hz, 1H), 7.72 (d, J = 8.7 Hz, 1H), 7.42 (s, 1H), 7.28 (d, J = 5.0 Hz, 1H), 6.97 (d, J = 8.7 Hz, 1H), 3.53 (s, 4H). |
| 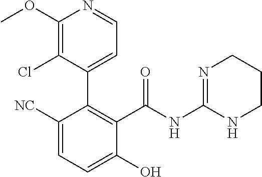 [SMILES: COc1nccc(-c2c(C#N)ccc(O)c2C(=O)NC2=NCCCN2)c1Cl] | 2-(3-chloro-2-methoxypyridin-4-yl)-3-cyano-6- hydroxy-N-(1,4,5,6-tetrahydropyrimidin-2- yl)benzamide LC-MS: m/z 386 (M + H)⁺. 1H NMR (400 MHz, DMSO) δ: 8.92 (s, 2H), 8.10 (d, J = 5.1 Hz, 1H), 7.71 (d, J = 8.7 Hz, 1H), 6.94 (d, J = 8.5 Hz, 1H), 6.89 (d, J = 5.1 Hz, 1H), 3.97 (s, 3H), 3.26 (t, J = 5.7 Hz, 4H), 1.83- 1.78 (m, 2H). |
| 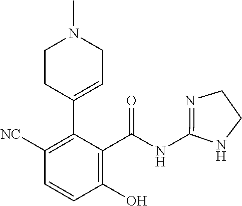 [SMILES: CN1CC=C(c2c(C#N)ccc(O)c2C(=O)NC2=NCCN2)CC1] | 3-cyano-N-(4,5-dihydro-1H-imidazol-2-yl)-6- hydroxy-2-(1-methyl-1,2,3,6-tetrahydropyridin- 4-yl)benzamide LC-MS: m/z 326 (M + H)⁺. 1H NMR (400 MHz, DMSO) δ 8.52 (br s, 2H), 7.57 (d, J = 8.8 Hz, 1H), 6.80 (d, J = 8.8 Hz, 1H), 5.33 (s, 1H), 3.58 (s, 4H), 2.94 (s, 2H), 2.61 (t, J = 5.6 Hz, 2H), 2.31-2.26 (m, 5H). |
| 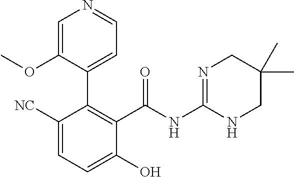 | 3-cyano-N-(5,5-dimethyl-1,4,5,6- tetrahydropyrimidin-2-yl)-6-hydroxy-2-(3- methoxypyridin-4-yl)benzamide LC-MS: m/z 380 (M + H)⁺. 1H NMR (400 MHz, DMSO) δ 8.37 (s, 1H), 8.20 (d, J = 4.7 Hz, 1H), 7.64 (d, J = 8.7 Hz, 1H), 7.06 (d, J = 4.7 Hz, 1H), 6.89 (d, J = 8.7 Hz, 1H), 3.78 (s, 3H), 2.95 (s, 4H), 0.93 (s, 6H). |
| 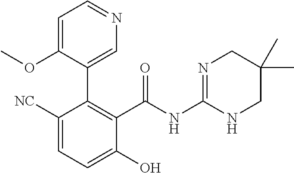 | 3-cyano-N-(5,5-dimethyl-1,4,5,6- tetrahydropyrimidin-2-yl)-6-hydroxy-2-(4- methoxypyridin-3-yl)benzamide LC-MS: m/z 380 (M + H)⁺. 1H NMR (400 MHz, DMSO) δ 8.97 (s, 2H), 8.41 (d, J = 5.7 Hz, 1H), 8.05 (s, 1H), 7.65 (d, J = 8.7 Hz, 1H), 7.09 (d, J = 5.8 Hz, 1H), 6.90 (d, J = 8.7 Hz, 1H), 3.75 (s, 3H), 2.96 (s, 4H), 0.94 (s, 6H). |
| 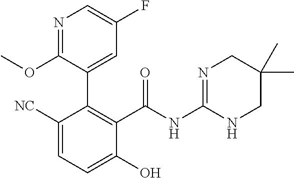 | 3-cyano-N-(5,5-dimethyl-1,4,5,6- tetrahydropyrimidin-2-yl)-2-(5-fluoro-2- methoxypyridin-3-yl)-6-hydroxybenzamide LC-MS: m/z 398 (M + H)⁺. 1H NMR (400 MHz, DMSO) δ 8.92 (br s, 2H), 8.09 (d, J = 2.8 Hz, 1H), 7.62 (d, J = 8.8 Hz, 1H), 7.52 (dd, J = 8.4, 2.8 Hz, 1H), 6.87 (d, J = 8.4 Hz, 1H), 3.74 (s, 3H), 2.95 (s, 4H), 0.93 (s, 6H). |
| 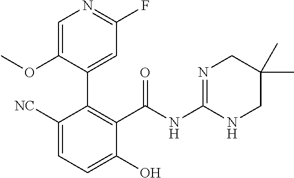 | 3-cyano-N-(5,5-dimethyl-1,4,5,6- tetrahydropyrimidin-2-yl)-2-(2-fluoro-5- methoxypyridin-4-yl)-6-hydroxybenzamide LC-MS: m/z 398 (M + H)⁺. 1H NMR (400 MHz, DMSO) δ 8.95 (s, 2H), 7.93 (d, J = 1.5 Hz, 1H), 7.68 (d, J = 8.7 Hz, 1H), 6.98 (d, J = 2.9 Hz, 1H), 6.92 (d, J = 8.7 Hz, 1H), 3.76 (s, 3H), 2.97 (s, 4H), 0.95 (s, 6H). |
| 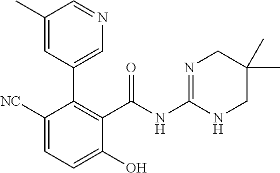 | 3-cyano-N-(5,5-dimethyl-1,4,5,6- tetrahydropyrimidin-2-yl)-6-hydroxy-2-(5- methylpyridin-3-yl)benzamide LC-MS: m/z 364 (M + H)⁺. 1H NMR (400 MHz, DMSO) δ 8.90 (br s, 2H), 8.35 (d, J = 1.6 Hz, 1H), 8.15 (d, J = 2.0 Hz, 1H), 7.64 (d, J = 8.8 Hz, 1H), 7.43 (s, 1H), 6.90 (d, J = 8.8 Hz, 1H), 2.95 (s, 4H), 2.32 (s, 3H), 0.94 (s, 6H). |
| 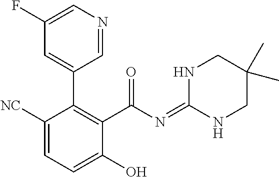 [SMILES: CC1(C)CNC(=NC(=O)c2c(O)ccc(C#N)c2-c2cncc(F)c2)NC1] | 3-cyano-N-(5,5-dimethyltetrahydropyrimidin- 2(1H)-ylidene)-2-(5-fluoropyridin-3-yl)-6- hydroxybenzamide LC-MS: m/z 368 (M + H)⁺. 1H NMR (400 MHz, DMSO) δ 8.96 (s, 2H), 8.52 (d, J = 2.7 Hz, 1H), 8.25 (t, J = 1.7 Hz, 1H), 7.70-7.65 (m, 2H), 6.93 (d, J = 8.7 Hz, 1H), 2.95 (s, 4H), 0.93 (s, 6H). |
| 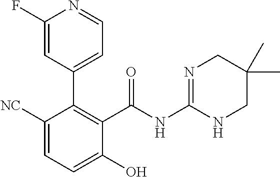 [SMILES: CC1(C)CN=C(NC(=O)c2c(O)ccc(C#N)c2-c2ccnc(F)c2)NC1] | 3-cyano-N-(5,5-dimethyl-1,4,5,6- tetrahydropyrimidin-2-yl)-2-(2-fluoropyridin-4- yl)-6-hydroxybenzamide LC-MS: m/z 368 (M + H)⁺. 1H NMR (400 MHz, DMSO) δ 8.92 (br s, 2H), 8.21 (d, J = 5.2 Hz, 1H), 7.65 (d, J = 8.8 Hz, 1H), 7.19 (d, J = 5.2 Hz, 1H), 7.08 (s, 1H), 6.90 (d, J = 8.8 Hz, 1H), 2.95 (s, 4H), 0.93 (s, 6H). |
| 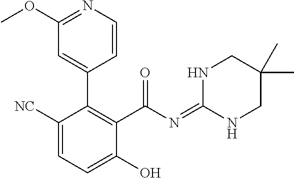 | 3-cyano-N-(5,5-dimethyltetrahydropyrimidin- 2(1H)-ylidene)-6-hydroxy-2-(2- methoxypyridin-4-yl)benzamide LC-MS: m/z 380 (M + H)⁺. 1H NMR (400 MHz, DMSO) δ 8.98 (s, 2H), 8.12 (d, J = 5.3 Hz, 1H), 7.64 (d, J = 8.7 Hz, 1H), 6.89 (d, J = 8.7 Hz, 1H), 6.79 (dd, J = 5.2, 1.4 Hz, 1H), 6.61 (d, J = 0.6 Hz, 1H), 3.87 (s, 3H), 2.95 (s, 4H), 0.94 (s, 6H). |
| 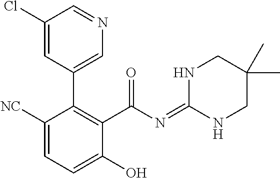 [SMILES: CC1(C)CNC(=NC(=O)c2c(O)ccc(C#N)c2-c2cncc(Cl)c2)NC1] | 2-(5-chloropyridin-3-yl)-3-cyano-N-(5,5- dimethyltetrahydropyrimidin-2(1H)-ylidene)-6- hydroxybenzamide LC-MS: m/z 384 (M + H)⁺. 1H NMR (400 MHz, DMSO) δ: 8.98 (s, 2H), 8.57 (d, J = 2.3 Hz, 1H), 8.33 (d, J = 1.8 Hz, 1H), 7.87-7.81 (m, 1H), 7.68 (d, J = 8.7 Hz, 1H), 6.94 (d, J = 8.7 Hz, 1H), 2.96 (s, 4H), 0.94 (s, 6H). |
| 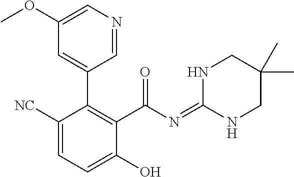 | 3-cyano-N-(5,5-dimethyltetrahydropyrimidin- 2(1H)-ylidene)-6-hydroxy-2-(5- methoxypyridin-3-yl)benzamide LC-MS: m/z 380 (M + H)⁺. 1H NMR (400 MHz, DMSO) δ 8.98 (s, 2H), 8.23 (d, J = 2.8 Hz, 1H), 7.95 (d, J = 1.7 Hz, 1H), 7.65 (d, J = 8.7 Hz, 1H), 7.23 (dd, J = 2.7, 1.8 Hz, 1H), 6.92 (d, J = 8.7 Hz, 1H), 3.83 (s, 3H), 2.95 (s, 4H), 0.94 (s, 6H). |
| 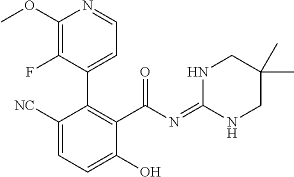 | 3-cyano-N-(5,5-dimethyltetrahydropyrimidin- 2(1H)-ylidene)-2-(3-fluoro-2-methoxypyridin- 4-yl)-6-hydroxybenzamide LC-MS: m/z 398 (M + H)⁺. 1H NMR (400 MHz, DMSO) δ 9.01 (s, 2H), 7.95 (d, J = 5.2 Hz, 1H), 7.71 (d, J = 8.8 Hz, 1H), 6.96 (d, J = 8.8 Hz, 1H), 6.88 (t, J = 4.8 Hz, 1H), 3.98 (s, 3H), 2.97 (s, 4H), 0.95 (s, 6H). |
| 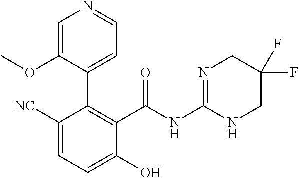 [SMILES: COc1cnccc1-c1c(C#N)ccc(O)c1C(=O)NC1=NCC(F)(F)CN1] | 3-cyano-N-(5,5-difluoro-1,4,5,6- tetrahydropyrimidin-2-yl)-6-hydroxy-2-(3- methoxypyridin-4-yl)benzamide LC-MS: m/z 388 (M + H)⁺. 1H NMR (400 MHz, DMSO) δ 9.29 (br s, 2H), 8.38 (s, 1H), 8.21 (d, J = 4.7 Hz, 1H), 7.72 (d, J = 8.7 Hz, 1H), 7.08 (d, J = 4.7 Hz, 1H), 6.98 (d, J = 8.7 Hz, 1H), 3.78 (s, 3H), 3.69 (t, J = 12.3 Hz, 4H). |
| 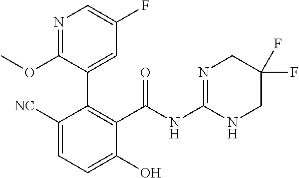 [SMILES: COc1ncc(F)cc1-c1c(C#N)ccc(O)c1C(=O)NC1=NCC(F)(F)CN1] | 3-cyano-N-(5,5-difluoro-1,4,5,6- tetrahydropyrimidin-2-yl)-2-(5-fluoro-2- methoxypyridin-3-yl)-6-hydroxybenzamide LC-MS: m/z 406 (M + H)⁺. 1H NMR (400 MHz, DMSO) δ9.30 (s, 2H), 8.11 (d, J = 2.8 Hz, 1H), 7.72 (d, J = 8.8 Hz, 1H), 7.56 (dd, J = 8.4, 2.8 Hz, 1H), 6.97 (d, J = 8.4 Hz, 1H), 3.74 (s, 3H), 3.69 (t, J = 12.4 Hz, 4H). |
| 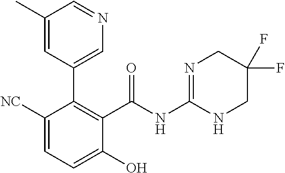 [SMILES: Cc1cncc(-c2c(C#N)ccc(O)c2C(=O)NC2=NCC(F)(F)CN2)c1] | 3-cyano-N-(5,5-difluoro-1,4,5,6- tetrahydropyrimidin-2-yl)-6-hydroxy-2-(5- methylpyridin-3-yl)benzamide LC-MS: m/z 372 (M + H)⁺. 1H NMR (400 MHz, DMSO) δ 9.28 (br s, 2H), 8.36 (s, 1H), 8.17 (d, J = 2.0 Hz, 1H), 7.73 (d, J = 8.4 Hz, 1H), 7.46 (s, 1H), 7.00 (d, J = 8.8 Hz, 1H), 3.69 (t, J = 12.4 Hz, 4H), 2.33 (s, 3H). |
| 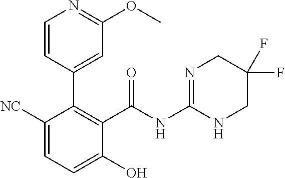 [SMILES: COc1cc(-c2c(C#N)ccc(O)c2C(=O)NC2=NCC(F)(F)CN2)ccn1] | 3-cyano-N-(5,5-difluoro-1,4,5,6- tetrahydropyrimidin-2-yl)-6-hydroxy-2-(2- methoxypyridin-4-yl)benzamide LC-MS: m/z 388 (M + H)⁺. 1H NMR (400 MHz, DMSO) δ 9.32 (s, 2H), 8.13 (d, J = 5.3 Hz, 1H), 7.71 (d, J = 8.7 Hz, 1H), 6.98 (d, J = 8.7 Hz, 1H), 6.81 (dd, J = 5.2, 1.4 Hz, 1H), 6.64 (s, 1H), 3.87 (s, 3H), 3.68 (t, J = 12.4 Hz, 4H). |

### General Procedure 11

Combination of the general procedures 5 and 8 described above.

The compounds in the table below were prepared from the appropriate starting materials described previously or commercially available using the above general procedure 11

| Structure | Name |
|---|---|
| 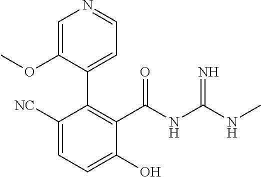 | 3-cyano-6-hydroxy-2-(3-methoxypyridin-4-yl)- N-(N-methylcarbamimidoyl)benzamide LC-MS: m/z 326 (M + H)⁺. 1H NMR (400 MHz, DMSO) δ 8.93 (br s, 1H), 8.37 (s, 1H), 8.20 (d, J = 4.7 Hz, 1H), 7.63 (d, J = 8.7 Hz, 1H), 7.06 (d, J = 4.5 Hz, 1H), 6.87 (d, J = 8.5 Hz, 1H), 3.78 (s, 3H), 2.73 (s, 3H). |
| 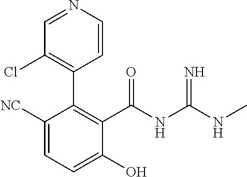 [SMILES: CNC(=N)NC(=O)c1c(O)ccc(C#N)c1-c1ccncc1Cl] | 2-(3-chloropyridin-4-yl)-3-cyano-6-hydroxy-N- (N-methylcarbamimidoyl)benzamide LC-MS: m/z 330 (M + H)⁺. 1H NMR (400 MHz, DMSO) δ 8.95 (d, J = 4.6 Hz, 1H), 8.65 (s, 1H), 8.52 (d, J = 4.9 Hz, 1H), 7.70 (d, J = 8.7 Hz, 1H), 7.32 (d, J = 4.9 Hz, 1H), 6.93 (d, J = 8.7 Hz, 1H), 2.74 (s, 3H). |
| 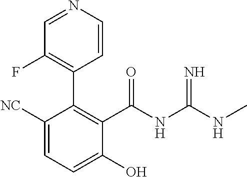 [SMILES: CNC(=N)NC(=O)c1c(O)ccc(C#N)c1-c1ccncc1F] | 3-cyano-2-(3-fluoropyridin-4-yl)-6-hydroxy-N- (N-methylcarbamimidoyl)benzamide LC-MS: m/z 314 (M + H)⁺. 1H NMR (400 MHz, DMSO) δ 9.02 (d, J = 3.9 Hz, 1H), 8.63 (s, 1H), 8.50 (d, J = 4.7 Hz, 1H), 7.77 (d, J = 8.7 Hz, 1H), 7.43-7.38 (m, 1H), 7.00 (d, J = 8.7 Hz, 1H), 2.80 (s, 3H). |
| 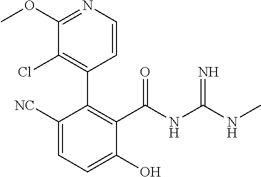 | 2-(3-chloro-2-methoxypyridin-4-yl)-3-cyano-6- hydroxy-N-(N-methylcarbamimidoyl)benzamide LC-MS: m/z 360 (M + H)⁺. 1H NMR (400 MHz, DMSO) δ: 8.95 (s, 1H), 8.46 (br s, 1H), 8.10 (d, J = 5.1 Hz, 1H), 7.68 (d, J = 8.7 Hz, 1H), 6.90 (t, J = 6.3 Hz, 2H), 3.97 (s, 3H), 2.74 (s, 3H). |
| 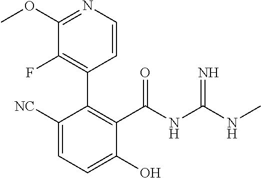 | 3-cyano-2-(3-fluoro-2-methoxypyridin-4-yl)-6- hydroxy-N-(N-methylcarbamimidoyl)benzamide LC-MS: m/z 344 (M + H)⁺. 1H NMR (400 MHz, DMSO) δ 7.96 (d, J = 5.1 Hz, 1H), 7.69 (d, J = 8.8 Hz, 1H), 6.93 (d, J = 8.8 Hz, 1H), 6.88 (t, J = 4.8 Hz, 1H), 3.98 (s, 3H), 2.75 (s, 3H). |
| 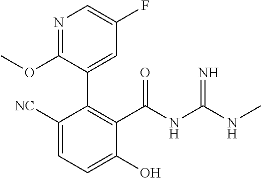 | 3-cyano-2-(5-fluoro-2-methoxypyridin-3-yl)-6- hydroxy-N-(N-methylcarbamimidoyl)benzamide LC-MS: m/z 344 (M + H)⁺. 1H NMR (400 MHz, DMSO) δ 8.20 (br s, 1H), 8.10 (d, J = 2.8 Hz, 1H), 7.64 (d, J = 8.8 Hz, 1H), 7.53 (dd, J = 8.4, 2.8 Hz, 1H), 6.87 (d, J = 8.4 Hz, 1H), 3.74 (s, 3H), 2.75 (s, 3H). |
| 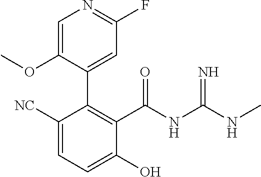 | 3-cyano-2-(2-fluoro-5-methoxypyridin-4-yl)-6- hydroxy-N-(N-methylcarbamimidoyl)benzamide LC-MS: m/z 344 (M + H)⁺. 1H NMR (400 MHz, DMSO) δ 8.21 (br s, 2H), 7.92 (d, J = 1.5 Hz, 1H), 7.65 (d, J = 8.7 Hz, 1H), 6.97 (d, J = 2.9 Hz, 1H), 6.88 (d, J = 8.7 Hz, 1H), 3.75 (s, 3H), 2.75 (s, 3H). |

### General Procedure 12

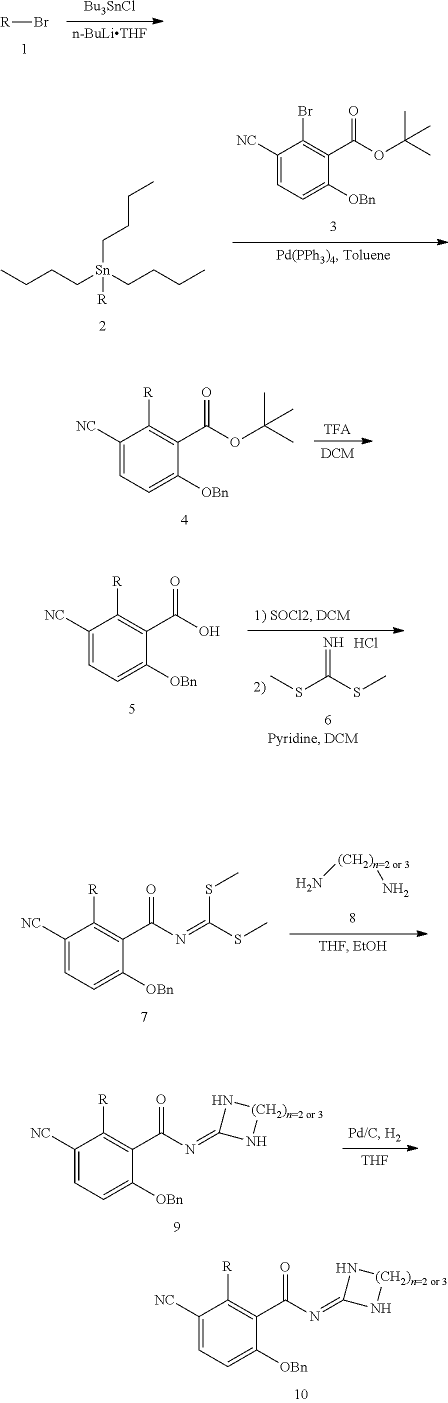

### Step 1: Synthesis of Compound 2

n-BuLi (1.1 eq.) was added dropwise at −20/−10° C. under N₂ atmosphere to a solution of compound 1 (1.0 eq.) in THF (0.1 mol/L), and the resulting mixture was stirred at −10° C. for 20 minutes before a solution of Bu₃SnCl (1.3 eq.) in anhydrous THF was added. The resulting mixture was then stirred at room temperature for 16 hours. The mixture was then quenched with saturated aq. NH₄Cl solution and extracted with EtOAc. The organic layer was separated, dried over Na₂SO₄, filtered and concentrated under vacuum. The residue was purified by column chromatography on silica gel (eluted with Petroleum Ether:EtOAc=100:0 to 25:1) to give compound 2.

### Step 2: Synthesis of Compound 4

Pd(PPh₃)₄ (0.1 eq.) was added under N₂ atmosphere to a suspension of 3 (1.0 eq.) and 2 (1.3 eq.) in toluene, and the resulting mixture was stirred at 100° C. for 20 hours. The mixture was then diluted with EtOAc and the organic layer was washed with water and brine, dried over Na₂SO₄, filtered and concentrated under vacuum. The residue was purified by column chromatography on silica gel (eluted with Petroleum Ether:EtOAc=10:1 to 5:1) to give compound 4.

### Step 3: Synthesis of Compound 5

TFA (10 eq.) was added under N₂ atmosphere to a solution of compound 4 (1 eq.) in DCM, and the resulting solution was stirred at room temperature for 3 hours. The reaction was then concentrated under vacuum and the residue was purified by column chromatography on silica gel (eluted with Petroleum Ether:EtOAc=10:1 to 1:1) to give compound 5.

### Step 4: Synthesis of Compound 7

Thionyl chloride (5 eq.) was added to a suspension of compound 5 (1 eq.) in anhydrous DCM, and the resulting mixture was stirred at 50° C. for 4-6 hours. After the reaction was completed, the mixture was concentrated under vacuum and the residue was then dissolved in anhydrous DCM and added to a mixture of compound 6 (1.5 eq.) and Pyridine (5 eq.) in dry DCM. The resulting mixture was stirred at room temperature for 1 hour and quenched with saturated aq. NaHCO₃ solution. The mixture was then extracted with EtOAc twice and the combined organic layers were washed with brine, dried over anhydrous Na₂SO₄, filtered and concentrated under vacuum. The residue was purified by column chromatography on silica gel (eluted with Petroleum Ether:EtOAc=10:1 to 2:1) to give compound 7.

### Step 5: Synthesis of Compound 9

The appropriate diamine 8 (2 eq.) was added under N₂ atmosphere to a mixture of compound 7 (1 eq.) in THF and EtOH (1:1, v/v), and the resulting mixture was stirred at 80° C. for 30 minutes. The mixture was then concentrated under vacuum to give compound 9.

### Step 6: Synthesis of Compound 10

10% Pd/C (20% w/w) was added to a solution of compound 9 (1 eq.) in THF, and the resulting mixture was stirred under 15 psi H₂ at room temperature for 30 minutes. The mixture was then filtered and the filtrate was concentrated under vacuum. The residue was purified by prep-HPLC (C18, 0% to 50% acetonitrile in H₂O with 0.1% NH₃.H₂O) to afford compound 10.

### Synthesis of A207

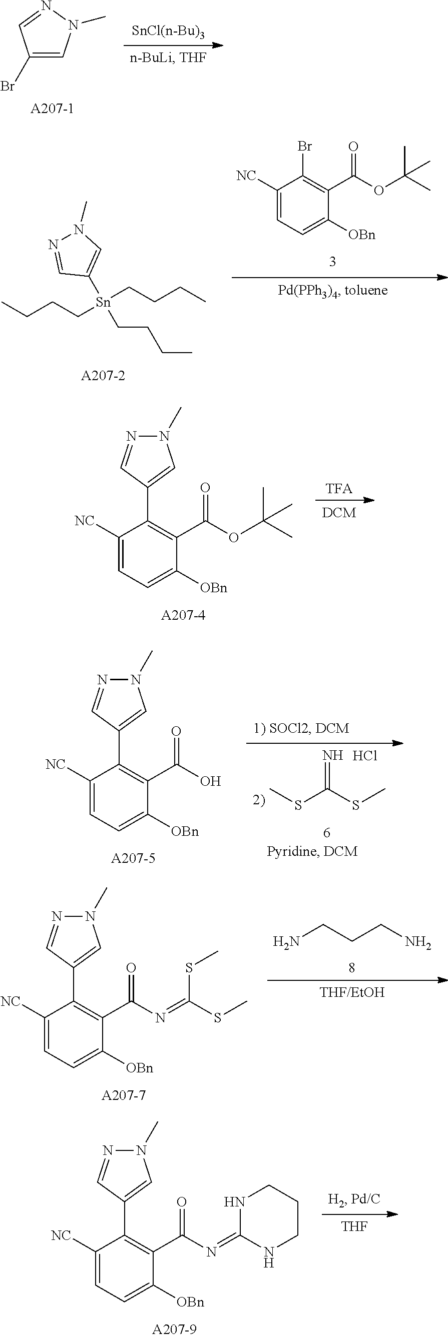

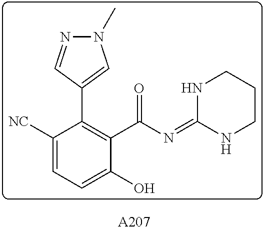

### Step 1: Synthesis of Compound A207-2

n-BuLi (8.2 mL, 8.2 mmol) was added dropwise at −20/−10° C. under N₂ atmosphere to a solution of compound A207-1 (1.2 g, 7.45 mmol) in THF (5 mL), and the resulting mixture was stirred at −10° C. for 20 minutes before a solution of Bu₃SnCl (3.15 g, 9.69 mmol) in anhydrous THF was added. The resulting mixture was stirred at room temperature for 16 hours. The mixture was then diluted with EtOAc and washed with saturated aq. NH₄Cl solution and brine, dried over Na₂SO₄, filtered and concentrated under vacuum. The residue was purified by column chromatography on silica gel (eluted with Petroleum Ether:EtOAc=100:0 to 25:1) to give compound A207-2 (1.02 g, 36.87% yield) as a white solid. LC/MS (ESI) m/z: 373 (M+H)⁺.

### Step 2: Synthesis of Compound A207-4

Pd(PPh₃)₄ (95.8 mg, 0.008 mmol) was added under N₂ atmosphere to a suspension of 3 (322 mg, 0.83 mmol) and A207-2 (400 mg, 1.08 mmol) in toluene, and the resulting mixture was stirred at 100° C. for 20 hours. The mixture was then diluted with EtOAc and washed with water and brine, dried over Na₂SO₄, filtered and concentrated under vacuum. The residue was purified by column chromatography on silica gel (eluted with Petroleum Ether:EtOAc=10:1 to 5:1) to give compound A207-4 (107 mg, 33.14% yield) as a white solid. LC/MS (ESI) m/z: 390 (M+H)⁺.

### Step 3: Synthesis of Compound A207-5

TFA (2 mL) was added under N₂ atmosphere to a mixture of compound A207-4 (107 mg, 0.27 mmol) in DCM (4 mL) and the resulting mixture was stirred at room temperature for 3 hours. The mixture was then concentrated under vacuum and the residue was purified by column chromatography on silica gel (eluted with Petroleum Ether:EtOAc=10:1 to 1:1) to give compound A207-5 (90 mg, 98.27% yield) as a white solid. LC/MS (ESI) m/z: 332 (M−H)⁻.

### Step 4: Synthesis of Compound A207-7

Thionyl chloride (160.6 mg, 1.35 mmol) was added to a suspension of compound A207-5 (90 mg, 270.0 μmol) in anhydrous DCM, and the resulting mixture was stirred at 50° C. for 4-6 hours. After the reaction was completed, the mixture was concentrated under vacuum and the crude product was dissolved in anhydrous DCM and added to a mixture of 6 (63.86 mg, 405.0 μmol) and Pyridine (106.8 mg, 1.35 mmol) in dry DCM. The resulting mixture was stirred at room temperature for 1 hour and quenched with saturated aq. NaHCO₃ solution. The mixture was then extracted with EtOAc and the organic layer was separated, dried over anhydrous Na₂SO₄, filtered and concentrated under vacuum. The residue was purified by column chromatography on silica gel (eluted with Petroleum Ether:EtOAc=10:1 to 2:1) to give compound A207-7 (87 mg, 73.81% yield) as a white solid. LC/MS (ESI) m/z: 437 (M+H)⁺.

### Step 5: Synthesis of Compound A207-9

Compound 8 (29.55 mg, 398.6 μmol) was added under N₂ atmosphere to a mixture of compound A207-7 (87 mg, 199.3 μmol) in THF (1.5 mL) and EtOH (1.5 mL), and the resulting mixture was stirred at 80° C. for 30 minutes. The mixture was then concentrated under vacuum to give compound A207-9 (81 mg, 98.06% yield) as a white solid. LC/MS (ESI) m/z: 415 (M+H)⁺.

### Step 6: Synthesis of Compound A207

10% Pd/C (16.2 mg) was added to a solution of compound A207-9 (81 mg, 195.4 μmol) in THF (3 mL), and the resulting mixture was stirred under H₂ atmosphere at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to afford compound A207 (50 mg, 78.88% yield) as a white solid. LC/MS (ESI) m/z: 325 (M+H)⁺.

The compounds in the table below were prepared from the appropriate starting materials described previously or commercially available using the above general procedure 12.

| Structure | Name |
|---|---|
| 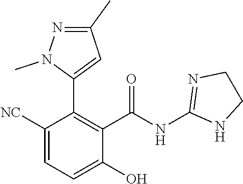 [SMILES: Cc1cc(-c2c(C#N)ccc(O)c2C(=O)NC2=NCCN2)n(C)n1] | 3-cyano-N-(4,5-dihydro-1H-imidazol-2-yl)-2- (1,3-dimethyl-1H-pyrazol-5-yl)-6- hydroxybenzamide LC-MS: m/z 325 (M + H)⁺. 1H NMR (400 MHz, DMSO) δ 8.63 (s, 2H), 7.71 (d, J = 8.7 Hz, 1H), 7.00 (d, J = 8.7 Hz, 1H), 5.92 (s, 1H), 3.55 (s, 4H), 3.40 (s, 3H), 2.15 (s, 3H). |
| 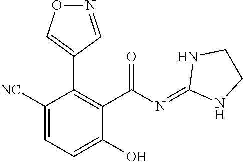 [SMILES: N#Cc1ccc(O)c(C(=O)N=C2NCCN2)c1-c1cnoc1] | 3-cyano-6-hydroxy-N-(imidazolidin-2-ylidene)-2- (isoxazol-4-yl)benzamide LC-MS: m/z 383 (M + H)⁺. 1H NMR (400 MHz, DMSO) δ 8.98 (s, 1H), 8.63(br s, 2H), 8.60 (s, 1H), 7.71 (d, J = 8.7 Hz, 1H), 6.97 (d, J = 8.7 Hz, 1H), 3.56(s, 4H). |
| 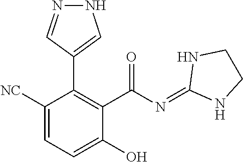 [SMILES: N#Cc1ccc(O)c(C(=O)N=C2NCCN2)c1-c1cn[nH]c1] | 3-cyano-6-hydroxy-N-(imidazolidin-2-ylidene)-2- (1H-pyrazol-4-yl)benzamide LC-MS: m/z 297 (M + H)⁺. 1H NMR (400 MHz, DMSO) δ 15.46 (s, 1H), 12.79 (s, 1H), 8.46 (s, 2H), 7.73 (s, 1H), 7.61 (d, J = 8.6 Hz, 1H), 7.44 (s, 1H), 6.86 (d, J = 8.6 Hz, 1H), 3.53 (s, 4H). |
| 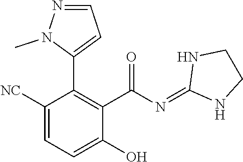 [SMILES: Cn1nccc1-c1c(C#N)ccc(O)c1C(=O)N=C1NCCN1] | 3-cyano-6-hydroxy-N-(imidazolidin-2-ylidene)-2- (1-methyl-1H-pyrazol-5-yl)benzamide LC-MS: m/z 311 (M + H)⁺. 1H NMR (400 MHz, DMSO) δ 8.63 (s, 2H), 7.74 (d, J = 8.7 Hz, 1H), 7.39 (d, J = 1.8 Hz, 1H), 7.02 (d, J = 8.7 Hz, 1H), 6.15 (d, J = 1.8 Hz, 1H), 3.56 (s, 4H), 3.50 (s, 3H). |
| 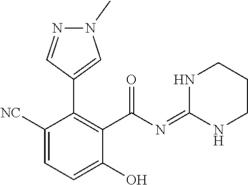 [SMILES: Cn1cc(-c2c(C#N)ccc(O)c2C(=O)N=C2NCCCN2)cn1] | 3-cyano-6-hydroxy-2-(1-methyl-1H-pyrazol-4- yl)-N-(tetrahydropyrimidin-2(1H)- ylidene)benzamide LC-MS: m/z 325 (M + H)⁺. 1H NMR (400 MHz, DMSO) δ 7.61 (d, J = 8.8 Hz, 1H), 7.36 (d, J = 1.8 Hz, 1H), 6.88 (d, J = 8.8 Hz, 1H), 6.10 (d, J = 1.8 Hz, 1H), 3.49 (s, 3H), 3.28-3.24 (m, 4H), 1.82-1.74 (m, 2H). |

### General Procedure 13

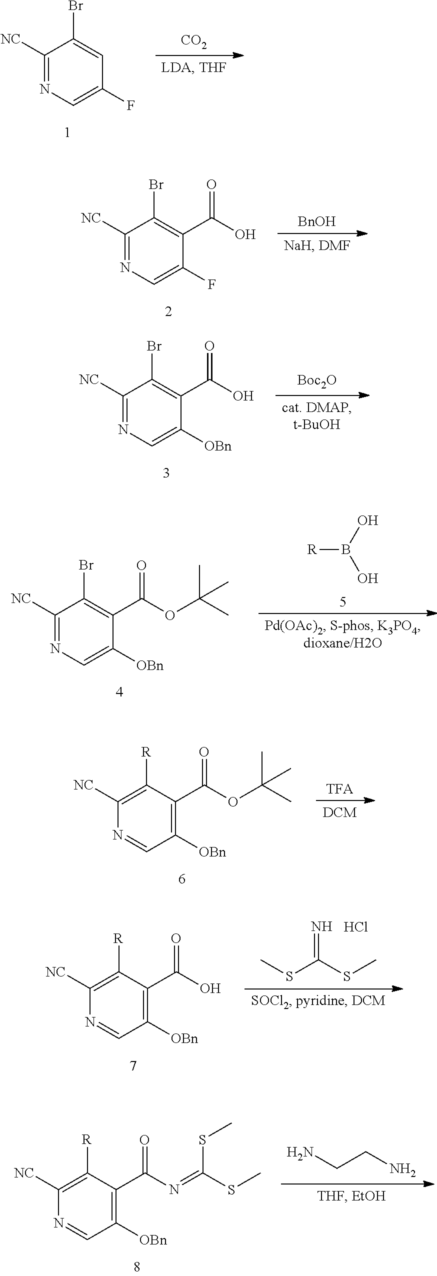

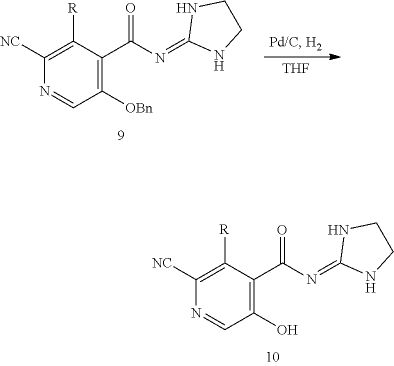

### Step 1: Synthesis of Compound 2

LDA (1.75 mL, 2M in THF) was added at −78° C. under N₂ atmosphere to a solution of compound 1 (500 mg, 2.5 mmol) in anhydrous THF (10 mL), and the resulting mixture was stirred at −78° C. for 1 hour. CO₂ (gas) was then bubbled into the above mixture at −78° C. for 30 minutes. When the reaction was completed a mixture of ice/water was added and the mixture was extracted with methyl tert-butyl ether twice. The aqueous layer was separated, acidified with 0.5 M aq. HCl solution and extracted with EtOAc twice. The combined organic layers were washed with brine, dried over Na₂SO₄, filtered and concentrated under vacuum to give compound 2 (320 mg, 52.46% yield) as a yellow solid. LC/MS (ESI) m/z: 243 (M−H)⁻.

### Step 2: Synthesis of Compound 3

60% NaH (110 mg, 2.75 mmol) was added at 0° C. under N₂ atmosphere to a solution of benzyl alcohol (0.2 mL, 1.967 mmol) in anhydrous DMF (10 mL), and the resulting mixture was stirred at room temperature for 1 hour before compound 2 (320 mg, 1.31 mmol) was added. The resulting mixture was then stirred at room temperature for 1.5 hours. The reaction mixture was then quenched with ice/H₂O and extracted with methyl tert-butyl ether twice. The aqueous layer was separated, acidified with 0.5 M aq.HCl solution and extracted with EtOAc twice. The combined organic layers were washed with brine, dried over anhydrous Na₂SO₄, filtered and concentrated to give compound 3 (420 mg, 96.46% yield) as a yellow solid without any further purification. LC/MS (ESI) m/z: 331 (M−H)⁻.

### Step 3: Synthesis of Compound 4

di-tert-butyl dicarbonate (0.6 mL, 2.53 mmol) and DMAP (23 mg, 0.19 mmol) were added to a solution of compound 3 (420 mg, 1.265 mmol) in t-BuOH (10 mL), and the resulting mixture was stirred at 60° C. for 16 hours. The mixture was then cooled to room temperature and concentrated under vacuum. The residue was purified by column chromatography on silica gel (Petroleum Ether:EtOAc=50:1 to 10:1) to give compound 4 (300 mg, 61.12% yield) as a white solid. LC/MS (ESI) m/z: 389 (M+H)+.

### Step 4: Synthesis of Compound 6

K₃PO₄ (2.5 eq) was added under N₂ atmosphere to a mixture of compound 4 (1.0 eq) and the appropriate phenylboronic acid 5 (1.5 eq) in dioxane and H₂O (8:1, V:V) followed by S-Phos (0.1 eq) and Pd(OAc)₂ (0.1 eq). The resulting reaction mixture was stirred at 95° C. for 16 hours under N₂ atmosphere. The mixture was then filtered and the filtrate was extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=50:1 to 10:1) to give compound 6.

### Step 5: Synthesis of Compound 7

TFA (2:1, v/v) was added at 0° C. to a solution of compound 6 (1.0 eq) in DCM and the mixture was stirred at room temperature for 2 hours. The mixture was then concentrated to give compound 7, which was used for next step without any further purification.

### Step 6: Synthesis of Compound 8

SOCl₂ (10.0 eq) was added at 0° C. to a solution of compound 7 (1.0 eq) in DCM, and the resulting mixture was stirred at 60° C. for 2 hours. The mixture was then concentrated under vacuum and the residue was added to a mixture of dimethyl carbonimidodithioate (1.5 eq) and anhydrous pyridine (1.5 eq) in dry DCM at 0° C. The reaction was then stirred at room temperature for 30 min and concentrated under vacuum. The residue was purified by column chromatography on silica gel (Petroleum Ether:EtOAc=10:1 to 5:1) to give compound 8.

### Step 7: Synthesis of Compound 9

The appropriate diamine (2.0 eq) was added to a solution of compound 8 (1.0 eq) in THF and EtOH (1:1, v/v) and the mixture was stirred at 80° C. for 1 hour. The mixture was then concentrated under vacuum and the residue was purified by column chromatography on silica gel (eluted with DCM:MeOH=80:1 to 30:1) to give compound 9.

### Step 8: Synthesis of Compound 10

10% Pd/C (1:1, w/w) was added to a solution of compound 9 (1.0 eq) in THF, and the mixture was degassed under N₂ atmosphere for three times and stirred under H₂ atmosphere at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to give compound 10.

### Synthesis of A392

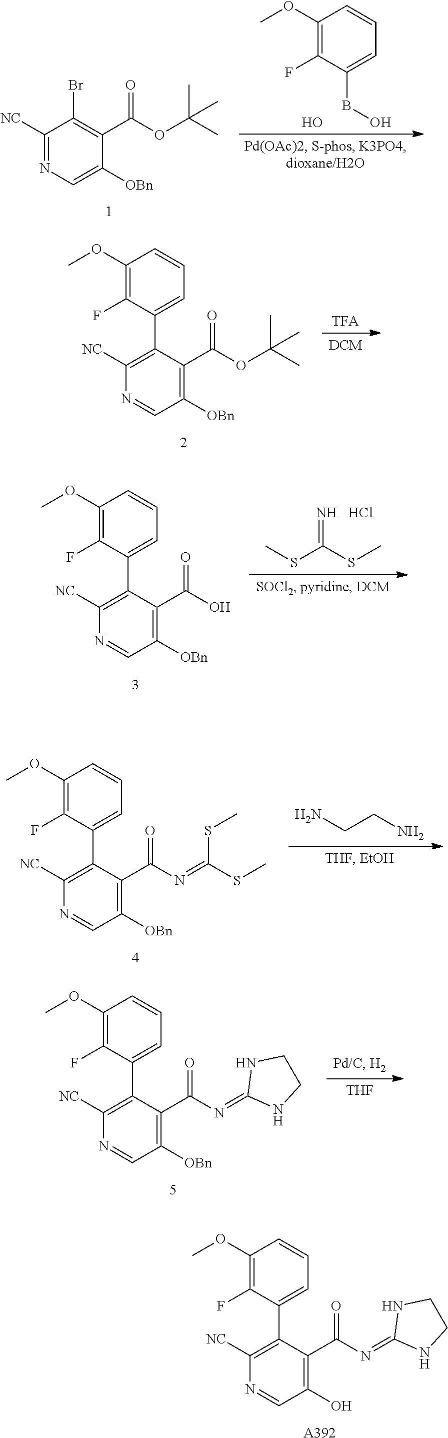

### Step 1: Synthesis of Compound A392-2

K₃PO₄ (369 mg, 1.74 mmol) was added under N₂ atmosphere to a mixture of compound A392-1 (270 mg, 0.70 mmol) and (2-fluoro-3-methoxyphenyl)boronic acid (177 mg, 1.04 mmol) in dioxane (8 mL) and H₂O (1 mL) followed by S-Phos (29 mg, 0.07 mmol) and Pd(OAc)₂ (16 mg, 0.07 mmol). The reaction mixture was stirred at 95° C. for 16 hours under N₂ atmosphere. The mixture was then filtered and the filtrate extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=50:1 to 10:1) to give compound A392-2 as a yellow solid (170 mg, 56.29% yield). LC/MS (ESI) m/z: 435 (M+H)⁺.

### Step 2: Synthesis of Compound A392-3

TFA (1.5 mL) was added at 0° C. to a solution of compound A392-2 (170 mg, 0.39 mmol) in DCM (3 mL) and the mixture was stirred at room temperature for 2 hours. The mixture was then concentrated under vacuum to give compound A392-3 as a yellow solid (148 mg, 99.96% yield) without any further purification. LC/MS (ESI) m/z: 377 (M−H)⁻.

### Step 3: Synthesis of Compound A392-4

Thionyl chloride (466 mg, 3.82 mmol) was added at 0° C. to a solution of compound A392-3 (148 mg, 0.38 mmol) in DCM (4 ml) and the mixture was then stirred at 60° C. for 2 hours. The mixture was concentrated under vacuum and the crude product was added to a mixture of dimethyl carbonimidodithioate (86 mg, 0.55 mmol) and anhydrous pyridine (47 mg, 0.55 mmol) in dry DCM (3 mL) at 0° C. The resulting mixture was then stirred at room temperature for 30 minutes and concentrated under vacuum. The residue was purified by column chromatography on silica gel (Petroleum Ether:EtOAc=10:1 to 5:1) to give compound A392-4 as a light yellow solid (90 mg, 47.79% yield). LC/MS (ESI) m/z: 482 (M+H)⁺.

### Step 4: Synthesis of Compound A392-5

Ethylenediamine (23 mg, 0.38 mmol) was added to a solution of compound A392-4 (90 mg, 0.19 mmol) in THF (2 mL) and EtOH (2 mL), and the reaction was stirred at 80° C. for 1 hour. The mixture was then concentrated under vacuum and the residue was purified by column chromatography on silica gel (DCM:MeOH=80:1 to 30:1) to give compound A392-5 as a light yellow solid (70 mg, 84.07% yield). LC/MS (ESI) m/z: 446 (M+H)⁺.

### Step 5: Synthesis of Compound A392

10% Pd/C (70 mg) was added to a solution of compound A392-5 (70 mg, 0.16 mmol) in THF (3 mL), and the mixture was degassed under N₂ atmosphere for three times and stirred under H₂ atmosphere at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to give compound A392 as a white solid (9.7 mg, 17.37% yield). LC/MS (ESI) m/z: 356 (M+H)⁺. ¹H NMR (400 MHz, DMSO) δ 9.01 (s, 2H), 8.34 (d, J=6.8 Hz, 1H), 7.24-7.18 (m, 2H), 6.93-6.85 (m, 1H), 3.92 (s, 3H), 3.64 (s, 4H).

The compounds in the table below were prepared from the appropriate starting materials described previously or commercially available using the above general procedure 13.

| Structure | Name |
|---|---|
| 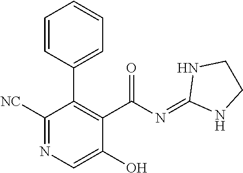 [SMILES: N#Cc1ncc(O)c(C(=O)N=C2NCCN2)c1-c1ccccc1] | 2-cyano-5-hydroxy-N-(imidazolidin-2-ylidene)-3- phenylisonicotinamide LC-MS: m/z 308 (M + H)+. 1H NMR (400 MHz, DMSO) δ 8.87 (s, 2H), 8.24 (s, 1H), 7.42-7.33 (m, 3H), 7.29-7.22 (m, 2H), 3.56 (s, 4H). |
| 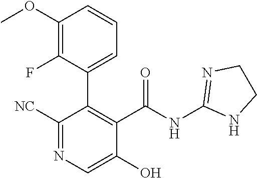 [SMILES: COc1cccc(-c2c(C#N)ncc(O)c2C(=O)NC2=NCCN2)c1F] | 2-cyano-N-(4,5-dihydro-1H-imidazol-2-yl)-3-(2- fluoro-3-methoxyphenyl)-5- hydroxyisonicotinamide LC-MS: m/z 356 (M + H)+. 1H NMR (400 MHz, DMSO) δ 9.01 (br s, 2H), 8.34 (d, J = 6.8 Hz, 1H), 7.24-7.18 (m, 2H), 6.93- 6.85 (m, 1H), 3.92 (s, 3H), 3.64 (s, 4H). |
| 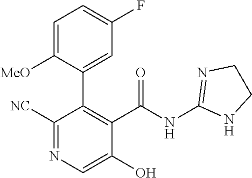 [SMILES: COc1ccc(F)cc1-c1c(C#N)ncc(O)c1C(=O)NC1=NCCN1] | 2-cyano-N-(4,5-dihydro-1H-imidazol-2-yl)-3-(5- fluoro-2-methoxyphenyl)-5- hydroxyisonicotinamide LC-MS: m/z 356 (M + H)+. 1H NMR (400 MHz, DMSO-d6) δ 8.88 (s, 2H), 8.23 (s, 1H), 7.18-7.13 (m, 1H), 7.06-7.02 (m, 2H), 3.66 (s, 3H), 3.57 (s, 4H). |
| 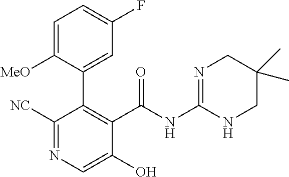 [SMILES: COc1ccc(F)cc1-c1c(C#N)ncc(O)c1C(=O)NC1=NCC(C)(C)CN1] | 2-cyano-N-(5,5-dimethyl-1,4,5,6- tetrahydropyrimidin-2-yl)-3-(5-fluoro-2- methoxyphenyl)-5-hydroxyisonicotinamide LC-MS: m/z 398 (M + H)+. 1H NMR (400 MHz, DMSO-d6) δ 9.16 (s, 2H), 8.15 (s, 1H), 7.18-7.12 (m, 1H), 7.07-6.96 (m, 2H), 3.66 (s, 3H), 2.98 (s, 4H), 0.94 (s, 6H). |
| 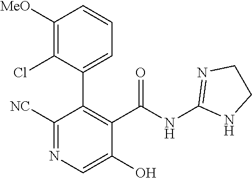 [SMILES: COc1cccc(-c2c(C#N)ncc(O)c2C(=O)NC2=NCCN2)c1Cl] | 3-(2-chloro-3-methoxyphenyl)-2-cyano-N-(4,5- dihydro-1H-imidazol-2-yl)-5- ydroxyisonicotinamide LC-MS: m/z 372 (M + H)⁺. 1H NMR(400 MHz, DMSO) δ 8.88 (s, 1H), 8.22 (s, 1H), 7.31 (dd, J = 8.0, 8.0 Hz, 1H), 7.12 (dd, J = 8.0, 1.2 Hz, 1H), 6.85 (dd, J = 8.0, 1.3 Hz, 1H), 3.88 (s, 3H), 3.54 (s, 4H) |

### General Procedure 14

### Method A:

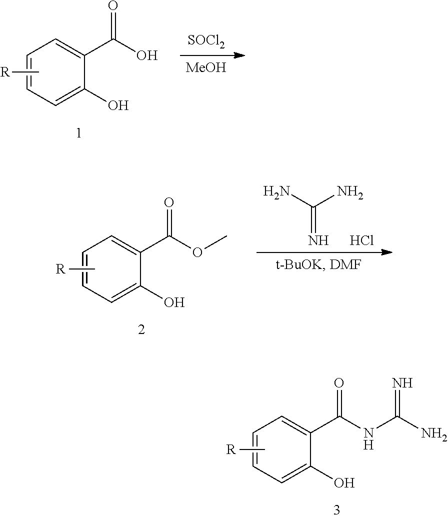

### Step 1: Synthesis of Compound 2

Thionyl chloride (10 eq.) was added at 0° C. to a solution of compound 1 (1 eq.) in MeOH and the resulting mixture was stirred at 80° C. under N₂ atmosphere for 16 hours. Then the mixture was concentrated under vacuum and the residue was purified by column chromatography on silica gel (eluted with Petroleum Ether:EtOAc=100:0 to 20:1) to give compound 2.

### Step 2: Synthesis of Compound 3

t-BuOK (8 eq.) was added under N₂ atmosphere to a suspension of guanidine hydrochloride (10 eq.) in dry DMF. The mixture was stirred at room temperature for 45 minutes before a solution of compound 2 (1 eq.) in DMF was added. The resulting mixture was stirred at room temperature for 16 hours. The mixture was diluted with EtOAc and washed with saturated aq. NH₄Cl solution and brine, dried over Na₂SO₄, filtered and concentrated under vacuum. The residue was purified via prep-HPLC (C18, 0% to 50% acetonitrile in H₂O with 0.1% NH₃.H₂O) to afford compound 3.

### Synthesis of A22

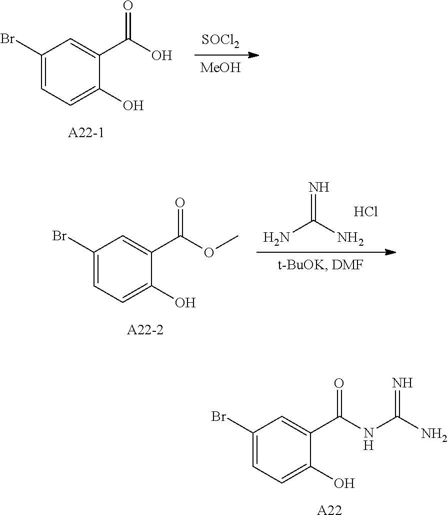

### Step 1: Synthesis of Compound A22-2

Thionyl chloride (2.38 g, 20 mmol) was added dropwise at 0° C. to a solution of compound A22-1 (432 mg, 2 mmol) in MeOH (20 mL), and the resulting mixture was stirred at 80° C. for 16 hours. The mixture was then concentrated under vacuum and the residue was purified by column chromatography on silica gel (eluted with Petroleum Ether:EtOAc=100:0 to 15:1) to give compound A22-2 (313 mg, 68% yield) as a colorless oil. LC/MS (ESI) m/z: 231 (M+H)⁺.

### Step 2: Synthesis of Compound A22

t-BuOK (1.22 g, 10.88 mmol) was added under N₂ atmosphere to a suspension of guanidine hydrochloride (1.30 g, 13.6 mmol) in dry DMF (25 mL). The resulting mixture was stirred at room temperature for 45 minutes before a solution of compound 2 (313 mg, 1.36 mmol) in DMF (2 mL) was added. The resulting mixture was stirred at room temperature for 16 hours. The mixture was then diluted with EtOAc and washed with saturated aq. NH₄Cl solution and brine, dried over Na₂SO₄, filtered and concentrated under vacuum. The residue was purified via prep-HPLC (C18, 0% to 50% acetonitrile in H₂O with 0.1% NH₃.H₂O) to give compound A22 (69 mg, 20% yield) as a white solid. LC/MS (ESI) m/z: 258 (M+H)⁺. 1H NMR (400 MHz, DMSO-d6) δ 14.94 (s, 1H), 8.37 (br s, 2H), 7.89 (d, J=2.7 Hz, 1H), 7.42 (dd, J=8.7, 2.7 Hz, 1H), 7.10 (br s, 1H), 6.76 (d, J=8.7 Hz, 1H).

### Method B:

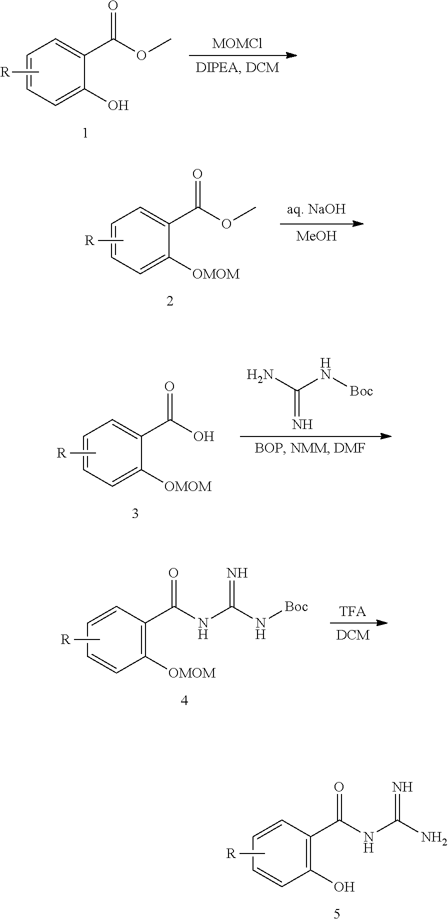

### Step 1: Synthesis of Compound 2

MOMCl (1.6 eq.) was added dropwise at 0° C. to a mixture of compound 1 (1 eq) and DIPEA (2.5 eq.) in dry DCM, and the resulting mixture was stirred at room temperature for 16 hours. The mixture was then quenched with saturated aq. NaHCO₃ solution and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=100:0 to 30:1) to give compound 2.

### Step 2: Synthesis of Compound 3

A solution of NaOH (3 eq.) in water was added to a mixture of compound 2 (1 eq.) in MeOH, and the resulting mixture was stirred at room temperature for 16 hours. The mixture was then diluted with water and extracted with diethylether. The aqueous layer was separated and acidified with 1 N aq. HCl solution to pH=5. The mixture was extracted with EtOAc twice and the combined organic layers were washed with brine, dried over anhydrous Na₂SO₄, filtered and concentrated under vacuum. The residue was purified by column chromatography on silica gel (eluted with Petroleum Ether:EtOAc=100:0 to 1:1) to give compound 3.

### Step 3: Synthesis of Compound 4

NMM (4 eq.) and PyBOP (1.5 eq.) were added under N₂ atmosphere to a mixture of compound 3 (1 eq.) and tert-butoxycarbonylguanidine (2 eq.) in dry DMF. The resulting mixture was stirred at room temperature for 16 hours. The mixture was then diluted with saturated aq. NH₄Cl solution and extracted with EtOAc twice. The combined organic layers were dried over anhydrous Na₂SO₄, filtered and concentrated under vacuum. The residue was purified by column chromatography on silica gel (eluted with Petroleum Ether:EtOAc=100:0 to 5:1) to give compound 4.

### Step 4: Synthesis of Compound 5

TFA (1:1, v/v) was added at 0° C. under N₂ atmosphere to a solution of compound 4 (1 eq.) in DCM, and the mixture was stirred at room temperature for 3 hours. The mixture was then concentrated under vacuum and the residue was purified via prep-HPLC (C18, 0% to 50% acetonitrile in H₂O with 0.1% NH₃.H₂O) to give compound 5.

### Synthesis of A43

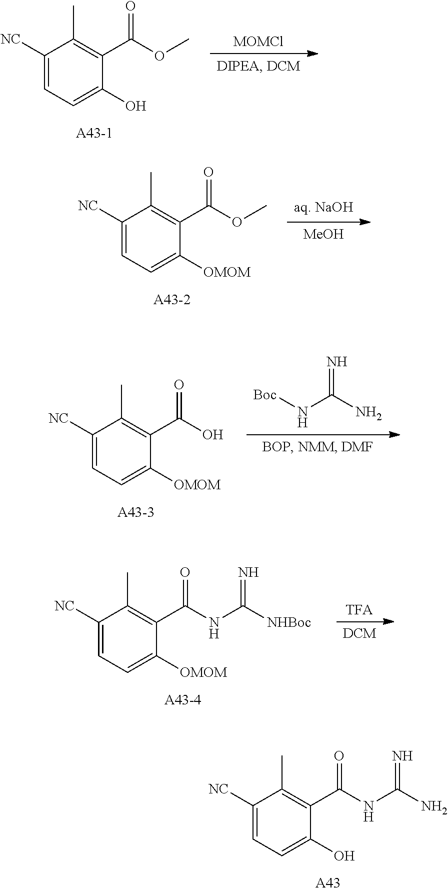

### Step 1: Synthesis of Compound A43-2

MOMCl (258 mg, 3.2 mmol) was added dropwise at 0° C. to a mixture of compound A43-1 (382 mg, 2 mmol) and DIPEA (650 mg, 5 mmol) in dry DCM (20 mL), and the resulting mixture was stirred at room temperature under N₂ atmosphere for 16 hours. The mixture was then quenched with saturated aq. NaHCO₃ solution and extracted with EtOAc twice. The organic layers were combined, dried over anhydrous Na₂SO₄, filtered and concentrated under vacuum. The residue was purified by column chromatography on silica gel (eluted with Petroleum Ether:EtOAc=100:0 to 30:1) to give compound A43-2 (310 mg, 66% yield) as a colorless oil. LC/MS (ESI) m/z: 258 (M+Na)⁺.

### Step 2: Synthesis of Compound A43-3

A solution of NaOH (158 mg, 3.96 mmol) in water (4 mL) was added to a solution of compound A43-2 (310 mg, 1.32 mmol) in MeOH (8 mL), and the resulting mixture was stirred at room temperature for 16 hours. The mixture was then diluted with water and extracted with ether. The aqueous layer was separated and acidified with 1 N aq. HCl solution to pH=5. The mixture was extracted with EtOAc twice and the combined organic layers were dried over anhydrous Na₂SO₄, filtered and concentrated under vacuum. The residue was purified by column chromatography on silica gel (eluted with Petroleum Ether:EtOAc=30:1 to 2:1) to give compound A43-3 (180 mg, 62% yield) as a colorless oil. LC/MS (ESI) m/z: 220 (M−H)⁻.

### Step 3: Synthesis of Compound A43-4

NMM (328 mg, 3.24 mmol) and PyBOP (537 mg, 1.22 mmol) were added under N₂ atmosphere to a mixture of compound A43-3 (180 mg, 0.81 mmol) and tert-butoxycarbonylguanidine (258 mg, 1.62 mmol) in dry DMF (8 mL). The resulting mixture was stirred at room temperature for 16 hours. The mixture was then diluted with saturated aq. NH₄Cl solution and extracted with EtOAc twice. The organic layers were combined, dried over anhydrous Na₂SO₄, filtered and concentrated under vacuum. The residue was purified by column chromatography on silica gel (eluted with Petroleum Ether:EtOAc=40:1 to 4:1) to give compound A43-4 (170 mg, 58% yield) as a colorless oil. LC/MS (ESI) m/z: 363 (M+H)⁺.

### Step 4: Synthesis of Compound A43

TFA (1 mL) was added at 0° C. under N₂ atmosphere to a solution of compound 6 (170 mg, 0.47 mmol) in DCM (2 mL). The resulting mixture was stirred at room temperature for 3 hours, and then the mixture was concentrated under vacuum. The residue was purified via prep-HPLC (C18, 0% to 50% acetonitrile in H₂O with 0.1% NH₃.H₂O) to give compound A43 (35 mg, 34% yield) as a white solid. LC/MS (ESI) m/z: 219 (M+H)⁺. 1H NMR (400 MHz, DMSO) δ 8.43 (br s, 2H), 7.51 (d, J=8.8 Hz, 1H), 7.30 (br s, 1H), 6.69 (d, J=8.8 Hz, 1H), 2.73 (s, 3H).

The compounds in the table below were prepared from the appropriate starting materials described previously or commercially available using the above general procedure 14 (method A or B).

| Structure | Name |
|---|---|
| 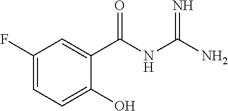 [SMILES: N=C(N)NC(=O)c1cc(F)ccc1O] | N-carbamimidoyl-5-fluoro-2- hydroxybenzamide LC-MS: m/z 198 (M + H)⁺. 1H NMR (400 MHz, DMSO) δ 14.47 (s, 1H), 8.35 (br s, 2H), 7.51-7.47 (m, 1H), 7.17-7.12 (m, 1H), 7.06 (br s, 1H), 6.80- 6.77 (m, 1H). |
| 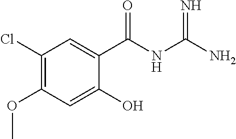 [SMILES: COc1cc(O)c(C(=O)NC(=N)N)cc1Cl] | N-carbamimidoyl-5-chloro-2-hydroxy-4- methoxybenzamide LC-MS: m/z 244 (M + H)⁺. 1H NMR (400 MHz, DMSO) δ 15.13 (s, 1H), 12.74 (s, 1H), 8.33 (br s, 2H), 7.72 (s, 1H), 7.04 (br s, 1H), 6.49 (s, 1H), 3.84 (s, 3H). |
| 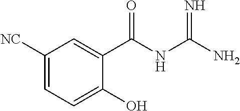 [SMILES: N#Cc1ccc(O)c(C(=O)NC(=N)N)c1] | N-carbamimidoyl-5-cyano-2- hydroxybenzamide LC-MS: m/z 205 (M + H)⁺. 1H NMR (400 MHz, DMSO) δ 8.49 (br s, 2H), 8.10 (s, 1H), 7.64 (d, J = 8.8 Hz, 1H), 7.34 (br s, 1H), 6.85 (d, J = 8.8 Hz, 1H). |
| 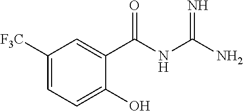 [SMILES: N=C(N)NC(=O)c1cc(C(F)(F)F)ccc1O] | N-carbamimidoyl-2-hydroxy-5- (trifluoromethyl)benzamide LC-MS: m/z 248 (M + H)⁺. 1H NMR (400 MHz, DMSO) δ 15.84 (s, 1H), 8.42 (br s, 2H), 8.09 (d, J = 4.0 Hz, 1H), 7.59 (d, J = 8.0 Hz, 1H), 7.24 (br s, 1H), 6.93 (d, J = 8.0 Hz, 1H). |
| 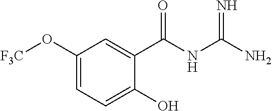 [SMILES: N=C(N)NC(=O)c1cc(OC(F)(F)F)ccc1O] | N-carbamimidoyl-2-hydroxy-5- (trifluoromethoxy)benzamide LC-MS: m/z 264 (M + H)⁺. 1H NMR (400 MHz, DMSO) δ 14.99 (s, 1H), 8.38 (br s, 2H), 7.69 (s, 1H), 7.29 (d, J = 8.0 Hz, 1H), 7.13 (br s, 1H), 6.87 (d, J = 8.0 Hz, 1H). |
| 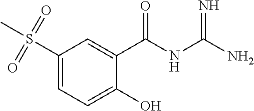 [SMILES: CS(=O)(=O)c1ccc(O)c(C(=O)NC(=N)N)c1] | N-carbamimidoyl-2-hydroxy-5- (methylsulfonyl)benzamide LC-MS: m/z 258 (M + H)⁺. 1H NMR (400 MHz, DMSO) δ 8.50 (br s, 2H), 8.30 (d, J = 2.8 Hz, 1H), 7.76 (dd, J = 8.8 Hz, 2.8 Hz, 1H), 7.29 (br s, 1H), 6.91 (d, J = 8.8 Hz, 1H), 3.12 (s, 3H). |
| 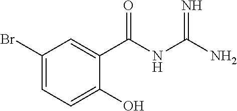 [SMILES: N=C(N)NC(=O)c1cc(Br)ccc1O] | 5-bromo-N-carbamimidoyl-2- hydroxybenzamide LC-MS: m/z 258 (M + H)⁺. 1H NMR (400 MHz, DMSO) δ 14.94 (s, 1H), 8.37 (br s, 2H), 7.89 (d, J = 2.7 Hz, 1H), 7.42 (dd, J = 8.7, 2.7 Hz, 1H), 7.10 (br s, 1H), 6.76 (d, J = 8.7 Hz, 1H). |
| 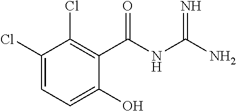 [SMILES: N=C(N)NC(=O)c1c(O)ccc(Cl)c1Cl] | N-carbamimidoyl-2,3-dichloro-6- hydroxybenzamide LC-MS: m/z 248 (M + H)⁺. 1H NMR (400 MHz, DMSO) δ 14.93 (br s, 1H), 8.20 (br s, 2H), 7.42 (d, J = 8.9 Hz, 1H), 7.14 (br s, 1H), 6.79 (d, J = 8.9 Hz, 1H). |
| 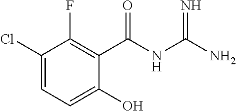 [SMILES: N=C(N)NC(=O)c1c(O)ccc(Cl)c1F] | N-carbamimidoyl-3-chloro-2-fluoro-6- hydroxybenzamide LC-MS: m/z 232 (M + H)⁺. 1H NMR (400 MHz, DMSO) δ 16.04 (s, 1H), 7.76 (br s, 3H), 7.38 (t, 1H), 6.63 (dd, J = 9.0, 1.5 Hz, 1H). |
| 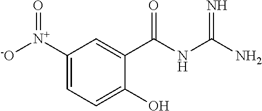 | N-carbamimidoyl-2-hydroxy-5- nitrobenzamide LC-MS: m/z 225 (M + H)⁺. 1H NMR (400 MHz, DMSO) δ 8.70 (br s, 2H), 8.65 (s, 1H), 8.06 (d, J = 7.1 Hz, 1H), 7.69 (br s, 1H), 6.72 (d, J = 9.2 Hz, 1H). |
| 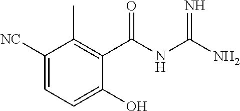 [SMILES: Cc1c(C#N)ccc(O)c1C(=O)NC(=N)N] | N-carbamimidoyl-3-cyano-6-hydroxy-2- methylbenzamide LC-MS: m/z 219 (M + H)⁺. 1H NMR (400 MHz, DMSO) δ 8.43 (br s, 2H), 7.51 (d, J = 8.8 Hz, 1H), 7.30 (br s, 1H), 6.69 (d, J = 8.8 Hz, 1H), 2.73 (s, 3H). |
| 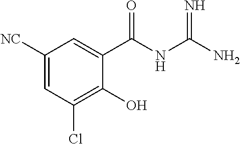 [SMILES: N#Cc1cc(Cl)c(O)c(C(=O)NC(=N)N)c1] | N-carbamimidoyl-3-chloro-5-cyano-2- hydroxybenzamide LC-MS: m/z 239 (M + H)⁺. 1H NMR (400 MHz, DMSO) δ 8.81 (br s, 2H), 7.97 (d, J = 2.3 Hz, 1H), 7.89 (br s, 1H), 7.77 (d, J = 2.3 Hz, 1H). |
| 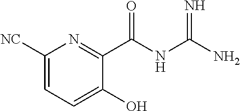 [SMILES: N#Cc1ccc(O)c(C(=O)NC(=N)N)n1] | N-carbamimidoyl-6-cyano-3- hydroxypicolinamide LC-MS: m/z 206 (M + H)⁺. 1H NMR (400 MHz, DMSO) δ 8.16 (br s, 3H), 7.73 (d, J = 8.1 Hz, 1H), 7.16 (d, J = 8.1 Hz, 1H). |
| 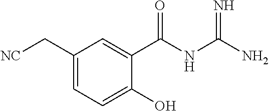 [SMILES: N#CCc1ccc(O)c(C(=O)NC(=N)N)c1] | N-carbamimidoyl-5-(cyanomethyl)-2- hydroxybenzamide LC-MS: m/z 219 (M + H)⁺. 1H NMR (400 MHz, DMSO) δ 14.82 (s, 1H), 8.36 (br s, 2H), 7.81 (d, J = 2.4 Hz, 1H), 7.24 (dd, J = 8.4, 2.5 Hz, 1H), 7.06 (br s, 1H), 6.79 (d, J = 8.4 Hz, 1H), 3.92 (s, 2H). |
| 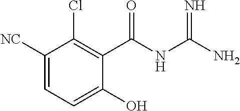 [SMILES: N#Cc1ccc(O)c(C(=O)NC(=N)N)c1Cl] | N-carbamimidoyl-2-chloro-3-cyano-6- hydroxybenzamide LC-MS: m/z 239 (M + H)⁺. 1H NMR (400 MHz, DMSO) δ 13.21 (br s, 1H), 8.58-8.28 (m, 4H), 7.85 (d, J = 8.8 Hz, 1H), 7.11 (d, J = 8.8 Hz, 1H). |
| 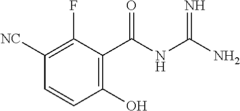 [SMILES: N#Cc1ccc(O)c(C(=O)NC(=N)N)c1F] | N-carbamimidoyl-3-cyano-2-fluoro-6- hydroxybenzamide LC-MS: m/z 223 (M + H)⁺. 1H NMR (400 MHz, DMSO) δ 8.44 (br s, 2H), 7.41 (t, J = 8.5 Hz, 1H), 6.47 (t, J = 11.6 Hz, 1H). |
| 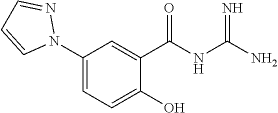 [SMILES: N=C(N)NC(=O)c1cc(-n2cccn2)ccc1O] | N-carbamimidoyl-2-hydroxy-5-(1H-pyrazol- 1-yl)benzamide LC-MS: m/z 246 (M + H)⁺. 1H NMR (400 MHz, DMSO) δ 14.86 (s, 1H), 8.39 (br s, 2H), 8.33 (d, J = 2.2 Hz, 1H), 8.19 (d, J = 2.8 Hz, 1H), 7.73 (dd, J = 8.8, 2.8 Hz, 1H), 7.67 (s, 1H), 7.08 (br s, 1H), |
| | 6.89 (d, J = 8.8 Hz, 1H), 6.47 (s, 1H). |
| 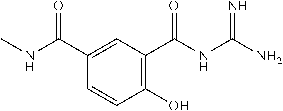 [SMILES: CNC(=O)c1ccc(O)c(C(=O)NC(=N)N)c1] | N3-carbamimidoyl-4-hydroxy-N1- methylisophthalamide LC-MS: m/z 237 (M + H)⁺. 1H NMR (400 MHz, DMSO) δ 15.52 (s, 1H), 8.37 (s, 1H), 8.26 (d, J = 3.7 Hz, 1H), 7.83-7.48 (m, 4H), 6.79 (d, J = 8.5 Hz, 1H), 2.75 (d, J = 3.8 Hz, 3H). |
| 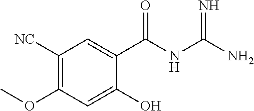 [SMILES: COc1cc(O)c(C(=O)NC(=N)N)cc1C#N] | N-carbamimidoyl-5-cyano-2-hydroxy-4- methoxybenzamide LC-MS: m/z 234 (M + H)⁺. 1H NMR (400 MHz, DMSO) δ 8.57 (br s, 2H), 7.96 (s, 1H), 7.38 (br s, 1H), 6.36 (s, 1H), 3.85 (s, 3H). |

### General Procedure 15

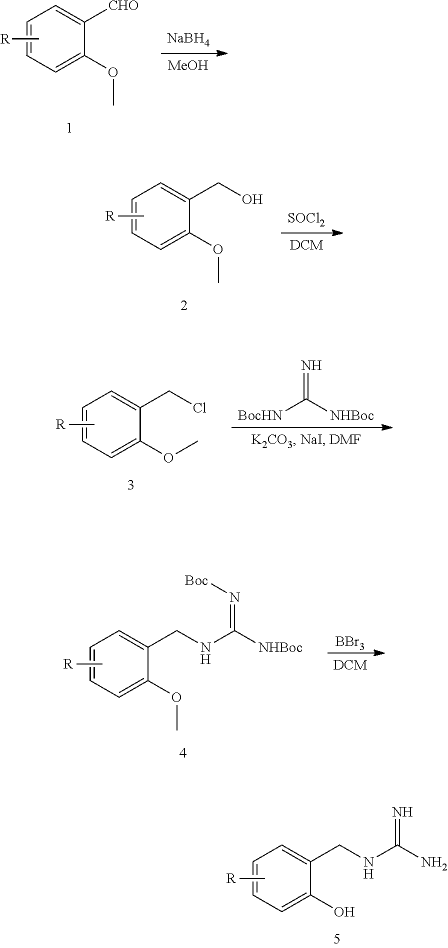

### Step 1: Synthesis of Compound 2

NaBH₄ (1.6 eq.) was added portion-wise at 0° C. to a solution of compound 1 (1 eq) in MeOH, and the resulting mixture was stirred at room temperature for 2 hours. The mixture was then quenched with saturated aq. NaHCO₃ solution and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=30:1 to 10:1) to give compound 2.

### Step 2: Synthesis of Compound 3

Thionyl chloride (3 eq.) was added dropwise at 0° C. to a solution of compound 2 (1 eq.) in dry DCM, and the resulting mixture was stirred at room temperature for 16 hours under N₂ atmosphere. The mixture was then concentrated under vacuum and the residue was purified by column chromatography on silica gel (eluted with Petroleum Ether:EtOAc=60:1 to 25:1) to give compound 3.

### Step 3: Synthesis of Compound 4

Potassium carbonate (2 eq.) and 1,3-Bis(tert-butoxycarbonyl)guanidine (1.5 eq.) were added to a mixture of compound 3 (1 eq.) and NaI (1 eq.) in DMF. The resulting mixture was stirred at 40° C. for 6 hours. The mixture was then cooled to room temperature, quenched with saturated aq. NH₄Cl solution and extracted with EtOAc twice. The combined organic layers were dried over anhydrous Na₂SO₄, filtered and concentrated under vacuum. The residue was purified by column chromatography on silica gel (eluted with Petroleum Ether:EtOAc=20:1 to 3:1) to give compound 4.

### Step 4: Synthesis of Compound 5

TFA (1:1, v/v) was added at 0° C. under N₂ atmosphere to a solution of compound 4 (1 eq.) in anhydrous DCM, and the resulting mixture was stirred at room temperature for 3 hours. The mixture was then concentrated under vacuum and the residue was purified via prep-HPLC (C18, 0% to 50% acetonitrile in H₂O with 0.1% NH₃.H₂O) to give compound 5.

### Synthesis of A2

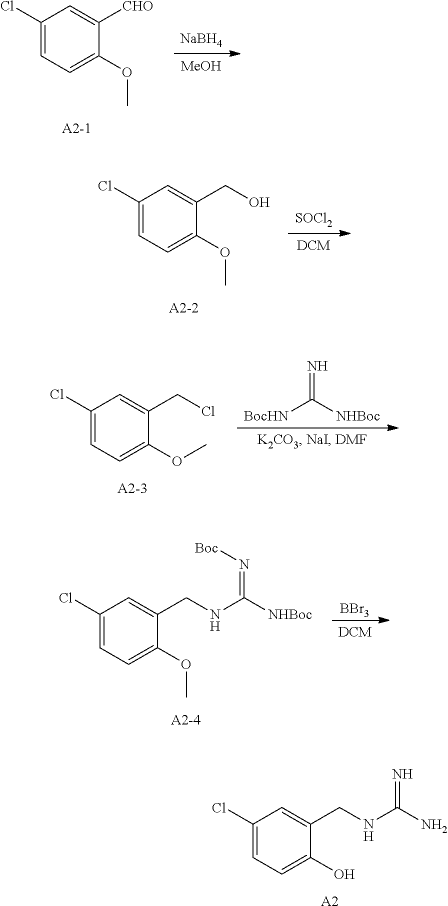

### Step 1: Synthesis of Compound A2-2

NaBH₄ (152 mg, 4 mmol) was added portion-wise at 0° C. to a solution of compound A2-1 (340 mg, 2 mmol) in MeOH (10 mL), and the resulting mixture was stirred at room temperature for 2 hours. The mixture was then quenched with saturated aq. NaHCO₃ solution and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=30:1 to 10:1) to give compound A2-2 (308 mg, 90% yield) as a colorless oil. LC/MS (ESI) m/z: 173 (M+H)⁺.

### Step 2: Synthesis of Compound A2-3

Thionyl chloride (640 mg, 5.37 mmol) was added dropwise at 0° C. to a solution of compound A2-2 (308 mg, 1.79 mmol) in dry DCM (8 mL), and the resulting mixture was stirred at room temperature for 16 hours under N₂ atmosphere. The mixture was then concentrated under vacuum and the residue was purified by column chromatography on silica gel (eluted with Petroleum Ether:EtOAc=60:1 to 25:1) to give compound A2-3 (223 mg, 65% yield) as a light-yellow solid. LC/MS (ESI) m/z: 213 (M+Na)⁺.

### Step 3: Synthesis of Compound A2-4

Potassium carbonate (323 mg, 2.34 mmol) and 1,3-Bis(tert-butoxycarbonyl)guanidine (454 mg, 1.76 mmol) were added to a mixture of compound A2-3 (223 mg, 1.17 mmol) and NaI (218 mg, 1.17 mmol) in DMF. The resulting mixture was stirred at 40° C. for 6 hours. The mixture was then cooled to room temperature, quenched with saturated aq. NH₄Cl solution and extracted with EtOAc twice. The combined organic layers were dried over anhydrous Na₂SO₄, filtered and concentrated under vacuum. The residue was purified by column chromatography on silica gel (eluted with Petroleum Ether:EtOAc=20:1 to 3:1) to give compound A2-4 (380 mg, 79% yield) as a white solid. LC/MS (ESI) m/z: 414 (M+H)⁺.

### Step 4: Synthesis of Compound A2

TFA (2 mL) was added at 0° C. under N₂ atmosphere to a solution of compound A2-4 (380 mg, 0.92 mmol) in anhydrous DCM (2 mL), and the resulting mixture was stirred at room temperature for 3 hours. The mixture was then concentrated under vacuum and the residue was purified via prep-HPLC (C18, 0% to 50% acetonitrile in H₂O with 0.1% NH₃.H₂O) to give compound A2 (33 mg, 18% yield) as a white solid. LC/MS (ESI) m/z: 200 (M+H)⁺. 1H NMR (400 MHz, MeOD) δ 8.53 (s, 2H), 7.21 (d, J=2.8 Hz, 1H), 7.16 (dd, J=8.8, 2.4 Hz, 1H), 6.82 (d, J=8.4 Hz, 1H), 4.32 (s, 2H).

The compounds in the table below were prepared from the appropriate starting materials described previously or commercially available using the above general procedure 15.

| Structure | Name |
|---|---|
| 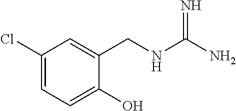 [SMILES: N=C(N)NCc1cc(Cl)ccc1O] | 1-(5-chloro-2-hydroxy- benzyl)guanidine LC-MS: m/z 200 (M + H)⁺. 1H NMR (400 MHz, MeOD) δ 8.53 (s, 2H), 7.21 (d, J = 2.8 Hz, 1H), 7.16 (dd, J = 8.8, 2.4 Hz, 1H), 6.82 |
| | (d, J = 8.4 Hz, 1H), 4.32 |
| | (s, 2H). |
| 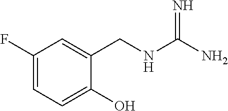 [SMILES: N=C(N)NCc1cc(F)ccc1O] | 1-(5-fluoro-2-hydroxy- benzyl)guanidine LC-MS: m/z 184 (M + H)⁺. 1H NMR (400 MHz, MeOD) δ 7.32-7.22 (m, 3H), 4.77 (s, 2H). |

### General Procedure 16

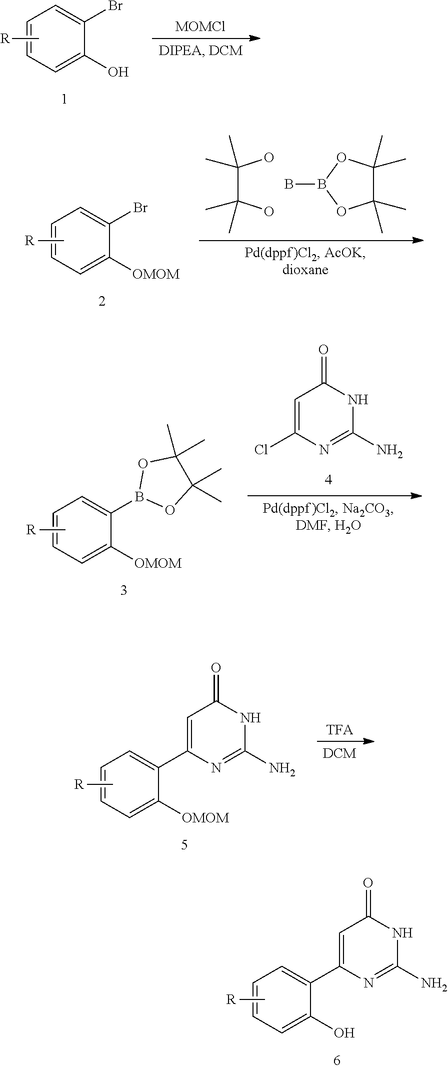

### Step 1: Synthesis of Compound 2

MOMCl (2 eq.) was added dropwise at 0° C. under N₂ atmosphere to a solution of compound 1 (1 eq) and DIPEA (3 eq.) in dry DCM. The resulting mixture was stirred at room temperature for 16 hours. The mixture was then quenched with saturated aq. NaHCO₃ solution and extracted with DCM twice. The combined organic layers were washed with brine, dried over anhydrous Na₂SO₄, filtered and concentrated under vacuum. The residue was purified by column chromatography on silica gel (eluted with Petroleum Ether:EtOAc=100:0 to 40:1) to give compound 2.

### Step 2: Synthesis of Compound 3

Potassium acetate (3 eq.) and Pd(dppf)Cl₂ (0.08 eq.) were added to a mixture of compound 2 (1 eq.) and bis(pinacolato)diboron (1.5 eq.) in 1,4-dioxane, and the resulting mixture was stirred at 100° C. for 16 hours under N₂ atmosphere. The mixture was then cooled to room temperature, diluted with EtOAc and washed with water. The organic layer was then dried over Na₂SO₄, filtered and concentrated under vacuum. The residue was purified by column chromatography on silica gel (eluted with Petroleum Ether:EtOAc=80:1 to 40:1) to give compound 3.

### Step 3: Synthesis of Compound 5

Sodium carbonate (3 eq.) and Pd(dppf)Cl₂ (0.08 eq.) were added to a mixture of compound 3 (1 eq.) and 2-amino-4-chloro-6-hydroxypyrimidine (1.5 eq.) in DMF and H₂O (10:1, v/v), and the resulting mixture was stirred at 90° C. for 16 hours under N₂ atmosphere. The mixture was then cooled to room temperature, diluted with EtOAc and washed with saturated aq. NH₄Cl solution. The organic layer was then dried over Na₂SO₄, filtered and concentrated under vacuum. The residue was purified by column chromatography on silica gel (eluted with Petroleum Ether:pEtOAc=50:1 to 4:1) to give compound 5.

### Step 4: Synthesis of Compound 6

TFA (1:1, v/v) was added dropwise at 0° C. under N₂ atmosphere to a solution of compound 5 (1 eq.) in anhydrous DCM, and the resulting mixture was stirred at room temperature for 3 hours. The mixture was then concentrated under vacuum and the residue was purified via prep-HPLC (C18, 0% to 50% acetonitrile in H₂O with 0.1% NH₃.H₂O) to give compound 6.

### Synthesis of C8

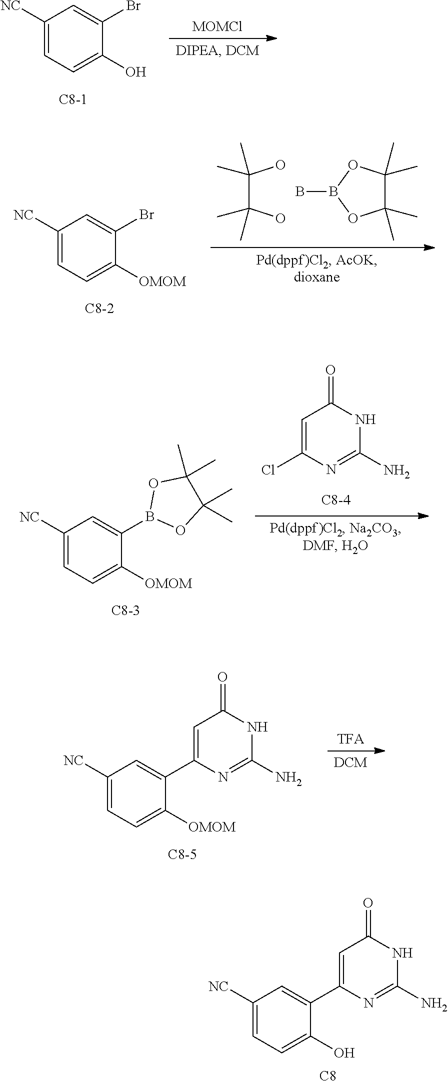

### Step 1: Synthesis of Compound C8-2

MOMCl (320 mg, 4 mmol) was added dropwise at 0° C. under N₂ atmosphere to a solution of compound C8-1 (396 mg, 2 mmol) and DIPEA (780 mg, 6 mmol) in dry DCM (10 mL). The resulting mixture was stirred at room temperature for 16 hours. The mixture was then quenched with saturated aq. NaHCO₃ solution and extracted with DCM twice. The combined organic layers were washed with brine, dried over anhydrous Na₂SO₄, filtered and concentrated under vacuum. The residue was purified by column chromatography on silica gel (eluted with Petroleum Ether:EtOAc=100:0 to 40:1) to give compound C8-2 (410 mg, 85% yield) as a colorless oil. LC/MS (ESI) m/z: 242 (M+H)⁺.

### Step 2: Synthesis of Compound C8-3

Potassium acetate (500 mg, 5.1 mmol) and Pd(dppf)Cl₂ (99 mg, 0.136 mmol) were added to a mixture of compound C8-2 (410 mg, 1.70 mmol) and bis(pinacolato)diboron (650 mg, 2.55 mmol) in 1,4-dioxane, and the resulting mixture was stirred at 100° C. for 16 hours under N₂ atmosphere. The mixture was then cooled to room temperature, diluted with EtOAc and washed with water. The organic layer was then dried over Na₂SO₄, filtered and concentrated under vacuum. The residue was purified by column chromatography on silica gel (eluted with Petroleum Ether:EtOAc=80:1 to 40:1) to give compound C8-3 (270 mg, 55% yield) as a yellow solid. LC/MS (ESI) m/z: 290 (M+H)⁺.

### Step 3: Synthesis of Compound C8-5

Sodium carbonate (296 mg, 2.79 mmol) and Pd(dppf)Cl₂ (54 mg, 0.074 mmol) were added to a mixture of compound C8-3 (270 mg, 0.93 mmol) and 2-amino-4-chloro-6-hydroxypyrimidine (203 mg, 1.4 mmol) in DMF and H₂O (11 mL, 10:1, v/v), and the resulting mixture was stirred at 90° C. for 16 hours under N₂ atmosphere. The mixture was then cooled to room temperature, diluted with EtOAc and washed with saturated aq. NH₄Cl solution. The organic layer was then dried over Na₂SO₄, filtered and concentrated under vacuum. The residue was purified by column chromatography on silica gel (eluted with Petroleum Ether:EtOAc=50:1 to 4:1) to give compound C8-5 (110 mg, 43% yield) as a yellow solid. LC/MS (ESI) m/z: 273 (M+H)⁺.

### Step 4: Synthesis of Compound C8

TFA (2 mL) was added dropwise at 0° C. under N₂ atmosphere to a solution of compound C8-5 (110 mg, 0.4 mmol) in anhydrous DCM (2 mL), and the resulting mixture was stirred at room temperature for 3 hours. The mixture was then concentrated under vacuum and the residue was purified via prep-HPLC (C18, 0% to 50% acetonitrile in H₂O with 0.1% NH₃.H₂O) to give compound C8 (12 mg, 13% yield) as a white solid. LC/MS (ESI) m/z: 229 (M+H)⁺. 1H NMR (400 MHz, DMSO-d6) δ 14.82 (br s, 1H), 11.23 (br s, 1H), 8.32 (d, J=2.0 Hz, 1H), 7.67 (dd, J=8.6, 2.0 Hz, 1H), 7.20 (s, 2H), 6.95 (d, J=8.6 Hz, 1H), 6.37 (s, 1H).

The compounds in the table below were prepared from the appropriate starting materials described previously or commercially available using the above general procedure 16.

| Structure | Name |
|---|---|
| 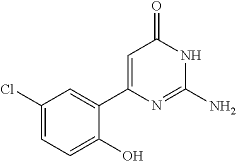 [SMILES: Nc1nc(-c2cc(Cl)ccc2O)cc(=O)[nH]1] | 2-amino-6-(5-chloro-2- hydroxyphenyl)pyrimidin- 4(3H)-one LC-MS: m/z 238 (M + H)⁺. 1H NMR (400 MHz, DMSO) δ 13.76 (s, 1H), 11.19 (s, 1H), 7.82 (d, J = 2.7 Hz, 1H), 7.30 (dd, J = 8.8, 2.6 Hz, 1H), 7.12 (br s, 2H), 6.84 (d, J = |
| | 8.8 Hz, 1H), 6.27 (s, 1H). |
| 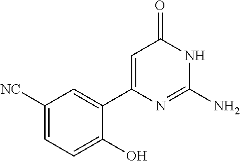 [SMILES: N#Cc1ccc(O)c(-c2cc(=O)[nH]c(N)n2)c1] | 3-(2-amino-6-oxo-1,6- dihydropyrimidin-4-yl)-4- hydroxybenzonitrile LC-MS: m/z 229 (M + H)⁺. 1H NMR (400 MHz, DMSO) δ 14.82 (br s, 1H), 11.23 (br s, 1H), 8.32 (d, J = 2.0 Hz, 1H), 7.67 (dd, J = 8.6, 2.0 Hz, 1H), 7.20 (s, 2H), 6.95 (d, J = |
| | 8.6 Hz, 1H), 6.37 (s, 1H). |
| 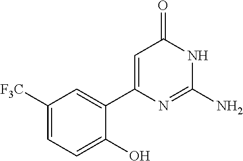 [SMILES: Nc1nc(-c2cc(C(F)(F)F)ccc2O)cc(=O)[nH]1] | 2-amino-6-(2-hydroxy-5- (trifluoromethyl)phenyl) pyrimidin-4(3H)-one LC-MS: m/z 272 (M + H)⁺. 1H NMR (400 MHz, DMSO-d6) δ 14.29 (s, 1H), 11.17 (s, 1H), 8.08 (s, 1H), 7.59 (dd, J = 8.7, 2.0 Hz, 1H), 7.13 (br s, 2H), 7.00 (d, J = 8.6 Hz, 1H), |
| | 6.36 (s, 1H). |

### General Procedure 17

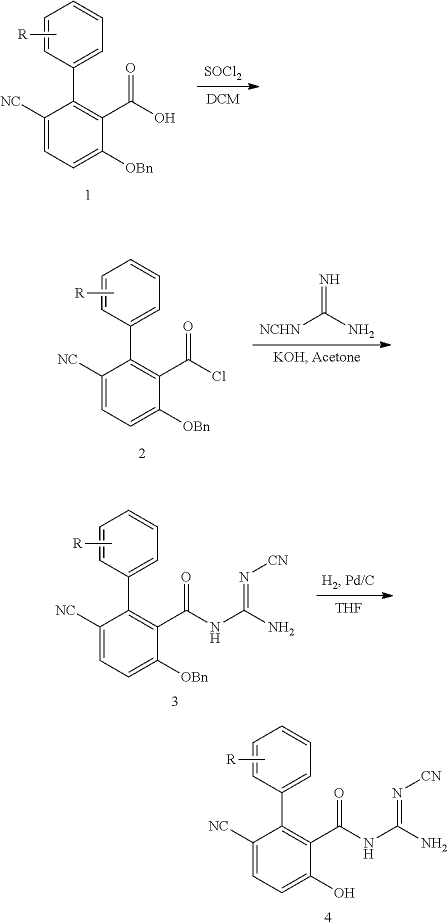

Intermediate 1 was prepared following general procedure 5

### Step 1: Synthesis of Compound 2

Thionyl chloride (10 eq) was added at 0° C. to a solution of compound 1 (1 eq) in DCM, and the mixture was stirred at 65° C. for 2 hours. The reaction mixture was then concentrated under vacuum to give compound 1 which was used in the next step without any further purification.

### Step 2: Synthesis of Compound 3

N-cyanoguanidine (1.25 eq) was added at 0° C. under N₂ atmosphere to a mixture of KOH (2 eq) in H₂O/Acetone, and the mixture was stirred at 10° C. for 1 hour. Then a solution of compound 2 (1 eq) in acetone was added dropwise to the above mixture and the resulting mixture was stirred at room temperature for 1 hour under N₂ atmosphere. The mixture was then poured into iced-water and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=15:1 to 4:1) to give compound 3.

### Step 3: Synthesis of Compound 4

10% Pd/C (1:1, w/w) was added at 0° C. to a solution of compound 3 (1 eq) in THF, and the mixture was degassed under N₂ atmosphere for three times and stirred under a H₂ at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to give compound 4

### Synthesis of A129

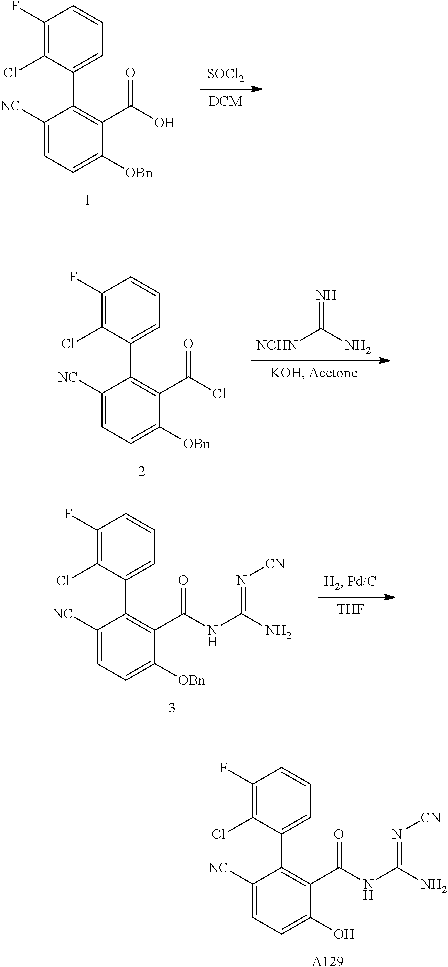

### Step 1: Synthesis of Compound A129-2

Thionyl chloride (0.2 mL, 3.0 mmol) was added at 0° C. to a solution of compound A129-1 (110 mg, 0.30 mmol) in DCM (4 mL), and the mixture was stirred at 65° C. for 2 hours. The reaction mixture was then concentrated under vacuum to give compound A129-2 (110 mg, 99% yield) as a light yellow solid which was used in the next step without any further purification.

### Step 2: Synthesis of Compound A129-3

N-cyanoguanidine (26 mg, 0.31 mmol) was added at 0° C. under N₂ atmosphere to a mixture of KOH (33 mg, 0.50 mmol) in H₂O (0.2 mL) and acetone (4 mL), and the mixture was stirred at 10° C. for 1 hour. Then a solution of compound A129-2 (100 mg, 0.25 mmol) in acetone (4 mL) was added dropwise to the above mixture and the resulting mixture was stirred at room temperature for 1 hour under N₂ atmosphere. The mixture was then poured into iced-water and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=15:1 to 4:1) to give compound A129-3 (62 mg, 55% yield) as a yellow solid. LC/MS (ESI) m/z: 448 (M+H)⁺.

### Step 3: Synthesis of Compound A129

10% Pd/C (60 mg) was added at 0° C. to a solution of compound A129-3 (60 mg, 0.13 mmol) in THF (4 mL), and the mixture was degassed under N₂ atmosphere for three times and stirred under a H₂ at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to give compound A129 (10 mg, 21% yield) as a white solid. LC/MS (ESI) m/z: 358 (M+H)⁺. ¹H NMR (400 MHz, DMSO-d6) δ 8.56 (s, 1H), 8.16 (s, 1H), 7.62 (d, J=8.8 Hz, 1H), 7.45-7.37 (m, 2H), 7.14-7.06 (m, 1H), 6.85 (d, J=8.4 Hz, 1H).

The compounds in the table below were prepared from the appropriate starting materials described previously or commercially available using the above general procedure 17.

| Structure | Name |
|---|---|
| 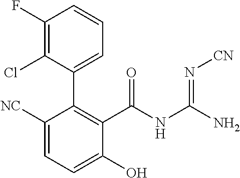 [SMILES: N#C/N=C(\N)NC(=O)c1c(O)ccc(C#N)c1-c1cccc(F)c1Cl] | 2′-chloro-6-cyano-N-(N′- cyanocarbamimidoyl)-3′- fluoro-3-hydroxy-[1,1′- biphenyl]-2-carboxamide LC/MS (ESI) m/z: 358 (M + H)⁺. 1H NMR (400 MHz, DMSO-d6) δ 8.56 (s, 1H), 8.16 (s, 1H), 7.62 (d, J = 8.8 Hz, 1H), 7.45-7.37 (m, 2H), 7.14-7.06 (m, |
| | 1H), 6.85 (d, J = 8.4 Hz, |
| | 1H). |
| 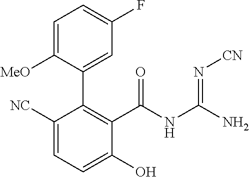 [SMILES: COc1ccc(F)cc1-c1c(C#N)ccc(O)c1C(=O)N/C(N)=N/C#N] | 6-cyano-N-(N′- cyanocarbamimidoyl)- 5′-fluoro-3- hydroxy-2′-methoxy- [1,1′-biphenyl]-2- carboxamide LC-MS: m/z 354 (M + H)⁺. 1H NMR (400 MHz, DMSO) δ 8.65 (s, 1H), 8.02 (s, 1H), 7.48 (d, J = |
| | 8.8 Hz, 1H), 7.12 (td, |
| | J = 8.8, 3.2 Hz, 1H), 7.01 |
| | (dd, J = 9.2, 4.8 Hz, 1H), |
| | 6.88 (dd, J = 8.8, 3.2 Hz, |
| | 1H), 6.71 (d, J = 8.4 Hz, |
| | 1H), 3.65 (s, 3H). |

### General Procedure 18

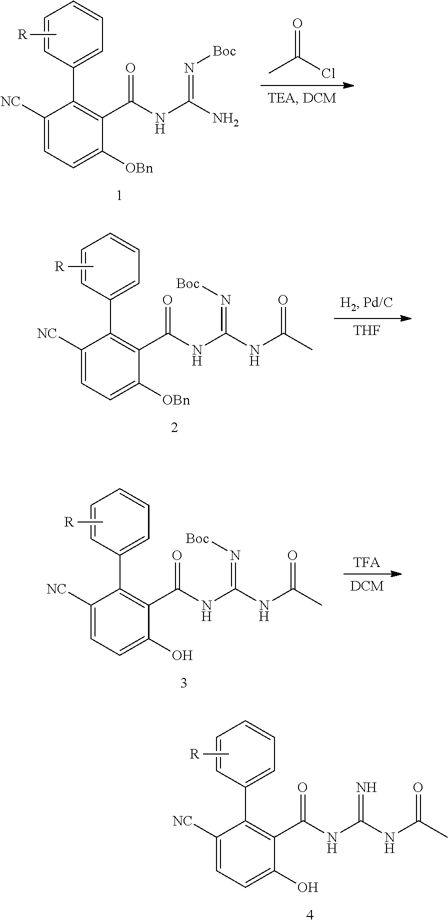

Intermediate 1 was prepared following procedure 5 and 7

### Step 1: Synthesis of Compound 2

TEA (1.5 eq) was added to a solution of compound 1 (1 eq) in DCM followed by dropwise addition of acetyl chloride (1.2 eq) at −20-10° C. under N₂ atmosphere. The resulting mixture was stirred at room temperature for 30 minutes. After the reaction was completed, the mixture was diluted with DCM and washed with H₂O and brine, dried over Na₂SO₄, filtered and concentrated under vacuum. The residue was purified by column chromatography on silica gel (eluted with Petroleum Ether:DCM=100:0 to 1:3) to give compound 2.

### Step 2: Synthesis of Compound 3

10% Pd/C (0.1 eq) was added to a solution of compound 2 (1 eq) in THF and the resulting mixture was degassed for three times under N₂ atmosphere and stirred under 15 psi H₂ for 30 minutes. The mixture was then filtered and the filtrate was concentrated under vacuum to give compound 3 without any further purification.

### Step 3: Synthesis of Compound 4

TFA (1:2, v/v) was added at 0° C. under N₂ atmosphere to a solution of compound 3 in DCM and the reaction was stirred at room temperature for 2 hours. The mixture was then concentrated under vacuum and the residue was purified via prep-HPLC (C18, 0% to 50% acetonitrile in H₂O with 0.1% NH₃.H₂O) to give compound 4.

### Synthesis of Compound A378

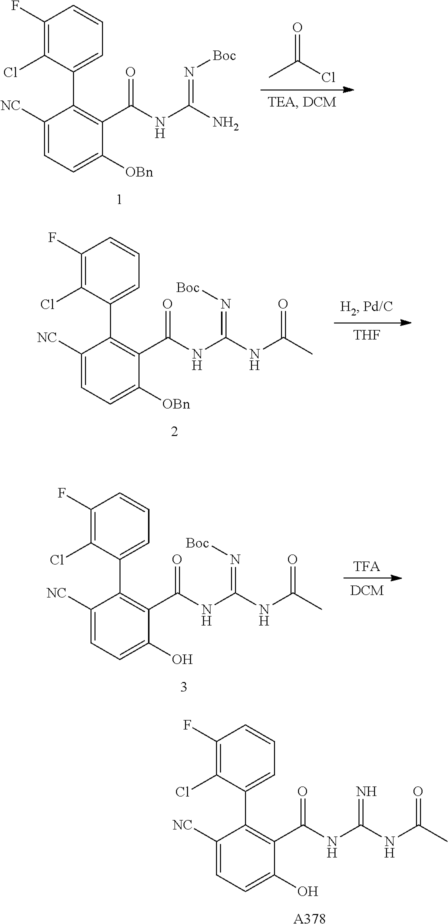

### Step 1: Synthesis of Compound A378-2

TEA (17.41 mg, 0.17 mmol) was added to a solution of compound A378-1 (60 mg, 0.114 mmol) in DCM (3 mL) followed by dropwise addition at −20-10° C. under N₂ atmosphere of acetyl chloride (10 mg, 0.126 mmol). The resulting mixture was stirred at room temperature for 30 minutes. After the reaction was completed, the mixture was diluted with DCM and washed with H₂O and brine, dried over Na₂SO₄, filtered and concentrated under vacuum. The residue was purified by column chromatography on silica gel (eluted with Petroleum Ether:DCM=100:0 to 1:3) to give compound A378-2 (32.0 mg, 49% yield) as a white solid. LC/MS (ESI) m/z: 565 (M+H)⁺.

### Step 2: Synthesis of Compound A378-3

10% Pd/C (16.0 mg) was added to a solution of compound A378-2 (32.0 mg, 0.057 mmol) in THF (3 mL) and the resulting mixture was degassed for three times under N₂ atmosphere and stirred under 15 psi H₂ for 30 minutes. The mixture was then filtered and the filtrate was concentrated under vacuum to give compound A378-3 (26.9 mg, 99% yield) as a white solid without any further purification. LC/MS (ESI) m/z: 475 (M+H)⁺.

### Step 5: Synthesis of Compound A378

TFA (2 mL) was added at 0° C. under N₂ atmosphere to a solution of compound A378-3 (26.9 mg, 0.056 mmol) in DCM (4 mL). The resulting mixture was stirred at room temperature for 2 hours. The mixture was then concentrated under vacuum and the residue was purified via prep-HPLC (C18, 0% to 50% acetonitrile in H₂O with 0.1% NH₃.H₂O) to afford compound A378 (4.5 mg, 21% yield) as a white solid. LC/MS (ESI) m/z: 375 (M+H)⁺. ¹H NMR (400 MHz, DMSO-d6) δ 14.96 (s, 1H), 11.38 (s, 1H), 9.79 (s, 1H), 9.39 (s, 1H), 7.85 (d, J=8.7 Hz, 1H), 7.42-7.37 (m, 2H), 7.14-7.09 (m, 2H), 2.16 (s, 3H).

The compounds in the table below were prepared from the appropriate starting materials described previously or commercially available using the above general procedure 18.

| Structure | Name |
|---|---|
| 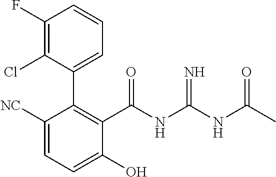 [SMILES: CC(=O)NC(=N)NC(=O)c1c(O)ccc(C#N)c1-c1cccc(F)c1Cl] | N-(N-acetylcarbamimidoyl)-2′-chloro-6-cyano-3′- fluoro-3-hydroxy-[1,1′-biphenyl]-2-carboxamide LC/MS (ESI) m/z: 375(M + H)⁺. 1H NMR (400 MHz, DMSO-d6) δ 14.96 (s, 1H), 11.38 (s, 1H), 9.79 (s, 1H), 9.39 (s, 1H), 7.85 (d, J = 8.7 Hz, 1H), 7.42-7.37 (m, 2H), 7.14-7.09 (m, 2H), 2.16 (s, 3H). |
| 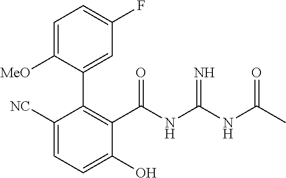 [SMILES: COc1ccc(F)cc1-c1c(C#N)ccc(O)c1C(=O)NC(=N)NC(C)=O] | N-(N-acetylcarbamimidoyl)-6-cyano-5′-fluoro-3- hydroxy-2′-methoxy-[1,1′-biphenyl]-2- carboxamide LC-MS: m/z 371 (M + H)⁺. 1H NMR (400 MHz, DMSO-d6) δ 9.60 (s, 1H), 9.42 (s, 1H), 7.62 (d, J = 8.6 Hz, 1H), 7.13-7.08 (m, 1H), 7.01 (dd, J = 9.1, 4.6 Hz, 1H), 6.95-6.84 (m, 2H), 3.64 (s, 3H), 2.09 (s, 3H). |
| 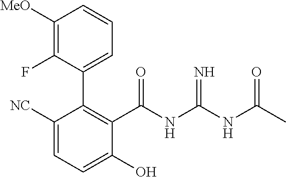 [SMILES: COc1cccc(-c2c(C#N)ccc(O)c2C(=O)NC(=N)NC(C)=O)c1F] | N-(N-acetylcarbamimidoyl)-6-cyano-2′-fluoro-3- hydroxy-3′-methoxy-[1,1′-biphenyl]-2- carboxamide LC/MS (ESI) m/z: 371(M + H)⁺. 1H NMR (400 MHz, DMSO-d6) δ 15.12 (s, 1H), 9.57 (s, 1H), 9.29 (s, 1H), 7.45 (d, J = 8.9 Hz, 1H), 7.08 (dd, J = 5.4, 2.4 Hz, 2H), 6.71-6.68 (m, 2H), 3.85 (s, 3H), 1.99 (s, 3H). |
| 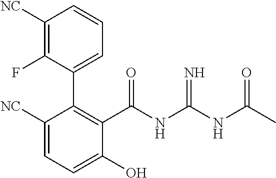 [SMILES: CC(=O)NC(=N)NC(=O)c1c(O)ccc(C#N)c1-c1cccc(C#N)c1F] | N-(N-acetylcarbamimidoyl)-3′,6-dicyano-2′- fluoro-3-hydroxy-[1,1′-biphenyl]-2-carboxamide LC/MS (ESI) m/z: 371(M + H)⁺. LC/MS (ESI) m/z: 366 (M + H)⁺. 1H NMR (400 MHz, DMSO-d6) δ 14.98 (s, 1H), 9.48 (s, 1H), 7.90 (dd, J = 8.0, 8.0 Hz, 1H), 7.70 (d, J = 8.0 Hz, 1H), 7.63 (dd, J = 8.0, 4.0 Hz, 1H), 7.45 (dd, J = 8.0, 4.0 Hz, 1H), 6.96 (d, J = 8.0 Hz, 1H), 2.09 (s, 3H). |

### General Procedure 19

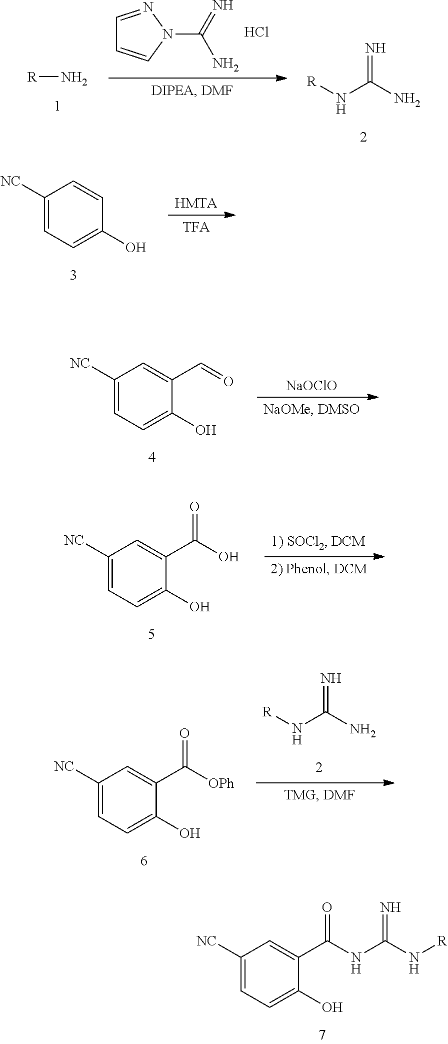

### Step 1: Synthesis of Compound 2

1H-pyrazole-1-carboxamidine hydrochloride (2 eq.) was added to a solution of the appropriate amine 1 (1 eq.) and DIPEA (8 eq.) in anhydrous DMF. The reaction mixture was stirred at room temperature overnight. MTBE was added to the reaction mixture and the mixture was stirred at room temperature for 20 minutes. The solvent was then removed and this procedure was repeated three to five times until the LC/MS showed a clean product. The appropriate alkylated acylguanidines 2 (10-20% yield) was obtained as a colorless oil or a white solid.

### Step 2: Synthesis of Compound 4

To a stirred solution of compound 3 (1 eq.) in TFA was added HMTA (2 eq.), and the resulting mixture was stirred at 100° C. for 6 hours. The mixture was then cooled to 0° C. and diluted sulfuric acid (10 mL of 98% H₂SO₄ in 130 mL of water) was added. The resulting mixture was stirred at room temperature for 30 minutes and extracted with DCM twice. The combined organic layers were washed with brine, dried over anhydrous Na₂SO₄, filtered and concentrated under vacuum. The residue was purified by column chromatography on silica gel (eluted with Petroleum Ether:EtOAc=10:1 to 3:1) to give compound 4 as a colorless oil. LC/MS (ESI) m/z: 148 (M+H)⁺.

### Step 3: Synthesis of Compound 5

NaOMe (1.1 eq.) was added to a stirred solution of compound 4 (1 eq.) in DMSO followed by portion-wise addition of NaOClO (2.2 eq.). The resulting mixture was stirred at room temperature for 4 hours. The mixture was then cooled to 0° C. and water was added slowly. The mixture was then acidified with 1 N aq. HCl solution to pH=2 and the mixture was extracted with EtOAc twice. The combined organic layers were washed with brine, dried over anhydrous Na₂SO₄, filtered and concentrated under vacuum. The residue was purified by column chromatography on silica gel (eluted with DCM:MeOH=100:1 to 20:1) to give compound 5 as a white solid. LC/MS (ESI) m/z: 162 (M−H)⁻.

### Step 4: Synthesis of Compound 6

Thionyl chloride (5 eq.) was added to a suspension of compound 5 (1 eq.) in anhydrous DCM, and the resulting mixture was stirred at 50° C. for 4-6 hours. After the reaction was completed, the mixture was concentrated under vacuum. The residue was then dissolved in anhydrous DCM and a solution of phenol (1 eq.) and Et₃N (3 eq.) in dry DCM was added. The resulting mixture was stirred at room temperature for 1 hour and then quenched with saturated aq. NaHCO₃ solution. The mixture was extracted with DCM twice and the combined organic layers were washed with brine, dried over anhydrous Na₂SO₄, filtered and concentrated under vacuum. The residue was purified by column chromatography on silica gel (eluted with Petroleum Ether:pEtOAc=100:0 to 40:1) to give compound 6 as a white solid. LC/MS (ESI) m/z: 238 (M−H)⁻.

### Step 5: Synthesis of Compound 7

Compound 2 (2 eq.) was added to a mixture of compound 6 (1 eq.) and 1,1,3,3-Tetramethylgyanidine (1.5 eq.) in DMF, and the resulting mixture was stirred at room temperature for 16 hours. The mixture was then diluted with water and extracted with EtOAc twice. The combined organic layers were washed with brine, dried over anhydrous Na₂SO₄, filtered and concentrated under vacuum. The residue was purified via prep-TLC to give compound 7.

### Synthesis of Compound F1

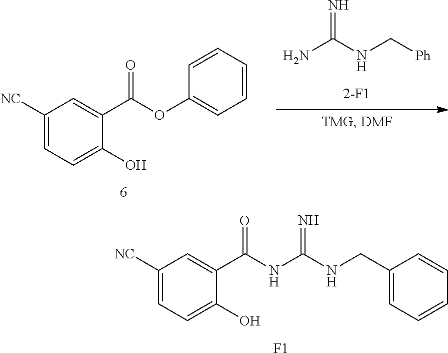

Compound 2-F1 (298 mg, 2.0 mmol) was added to a mixture of compound 6 (239 mg, 1 mmol) and 1,1,3,3-Tetramethylgyanidine (172 mg, 1.5 mmol) in DMF (5 mL), and the resulting mixture was stirred at room temperature for 16 hours. The mixture was then diluted with water and extracted with EtOAc twice. The combined organic layers were washed with brine, dried over anhydrous Na₂SO₄, filtered and concentrated under vacuum. The residue was purified via prep-TLC to give compound F1 (60 mg, 20% yield) as a white solid. LC/MS (ESI) m/z: 295 (M+H)⁺. ¹H NMR (400 MHz, MeOD) δ 8.20 (s, 1H), 7.55 (d, J=8.8 Hz, 1H), 7.43-7.27 (m, 5H), 6.86 (d, J=8.7 Hz, 1H), 4.51 (s, 2H).

The compounds in the table below were prepared from the appropriate starting materials described previously or commercially available using the above general procedure 19.

| Structure | Name |
|---|---|
| 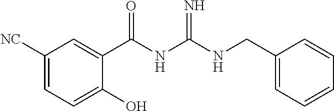 [SMILES: N#Cc1ccc(O)c(C(=O)NC(=N)NCc2ccccc2)c1] | N-(N-benzylcarbamimidoyl)-5-cyano- 2-hydroxybenzamide LC-MS: m/z 295 (M + H)⁺. 1H NMR (400 MHz, MeOD) δ 8.20 (s, 1H), 7.55 (d, J = 8.8 Hz, 1H), 7.43- 7.27 (m, 5H), 6.86 (d, J = 8.7 Hz, 1H), 4.51 (s, 2H). |
| 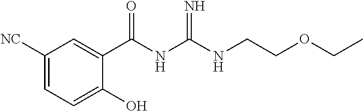 [SMILES: CCOCCNC(=N)NC(=O)c1cc(C#N)ccc1O] | 5-cyano-N-(N-(2- ethoxyethyl)carbamimidoyl)-2- hydroxybenzamide LC-MS: m/z 277 (M + H)⁺. 1H NMR (400 MHz, DMSO-d6) δ 16.16 (s, 1H), 8.11 (s, 1H), 7.66 (d, J = 8.4 Hz, 1H), 6.88 (d, J = 8.6 Hz, 1H), |
| | 3.53 (t, J = 5.3 Hz, 2H), 3.49 (q, J = 7.0 |
| | Hz, 2H), 3.38 (t, J = 5.3 Hz, 2H), 1.14 |
| | (t, J = 7.0 Hz, 3H). |
| 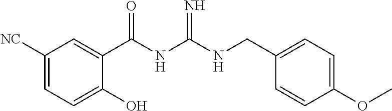 [SMILES: COc1ccc(CNC(=N)NC(=O)c2cc(C#N)ccc2O)cc1] | 5-cyano-2-hydroxy-N-(N-(4- methoxybenzyl)carbamimidoyl)benzamide LC-MS: m/z 325 (M + H)⁺. 1H NMR (400 MHz, DMSO-d6) δ 16.08 (s, 1H), 9.63 (s, 1H), 8.80 (s, 1H), 8.11 (s, 1H), 7.66 (d, J = 8.4 Hz, 1H), 7.30 (d, J = 8.3 Hz, 2H), 6.94 (d, J = 8.5 |
| | Hz, 2H), 6.88 (d, J = 8.6 Hz, 1H), 4.38 |
| | (s, 2H), 3.74 (s, 3H). |
| 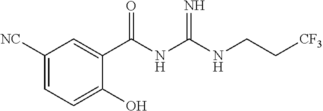 [SMILES: N#Cc1ccc(O)c(C(=O)NC(=N)NCCC(F)(F)F)c1] | 5-cyano-2-hydroxy-N-(N-(3,3,3- trifluoropropyl)carbamimidoyl)benzamide LC-MS: m/z 301 (M + H)⁺. 1H NMR (400 MHz, DMSO-d6) δ 16.01 (s, 1H), 8.13 (s, 1H), 7.68 (d, J = 7.7 Hz, 1H), 6.89 (d, J = 7.7 Hz, 1H), 3.49 (t, J = 6.8 Hz, 2H), 2.68-2.57 (m, |
| | 2H). |
| 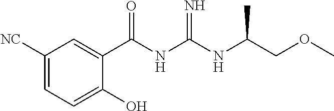 [SMILES: COC[C@H](C)NC(=N)NC(=O)c1cc(C#N)ccc1O] | (S)-5-cyano-2-hydroxy-N-(N-(1- methoxypropan-2- yl)carbamimidoyl)benzamide LC-MS: m/z 277 (M + H)⁺. 1H NMR (400 MHz, DMSO-d6) δ 16.12 (s, 1H), 9.35 (s, 1H), 8.11 (s, 1H), 7.66 (d, J = 7.6 Hz, 1H), 6.88 (d, J = 8.5 |
| | Hz, 1H), 4.01-3.82 (m, 1H), 1.15 (d, J = |
| | 6.2 Hz, 3H). |
| 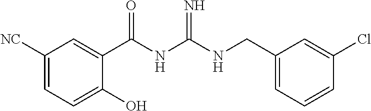 [SMILES: N#Cc1ccc(O)c(C(=O)NC(=N)NCc2cccc(Cl)c2)c1] | N-(N-(3-chlorobenzyl)carbamimidoyl)- 5-cyano-2-hydroxybenzamide LC-MS: m/z 329 (M + H)⁺. 1H NMR (400 MHz, DMSO-d6) δ 16.01 (s, 1H), 9.64 (s, 1H), 8.90 (s, 1H), 8.14 (s, 1H), 7.88 (s, 1H), 7.68 (d, J = 8.4 Hz, 1H), 7.47-7.26 (m, 4H), 6.90 |
| | (d, J = 8.6 Hz, 1H), 4.48 (s, 2H). |
| 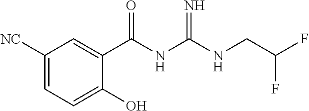 [SMILES: N#Cc1ccc(O)c(C(=O)NC(=N)NCC(F)F)c1] | 5-cyano-N-(N-(2,2- difluoroethyl)carbamimidoyl)-2- hydroxybenzamide LC-MS: m/z 269 (M + H)⁺. 1H NMR (400 MHz, DMSO-d6) δ 15.71 (s, 1H), 8.14 (s, 1H), 7.69 (d, J = 7.7 Hz, 1H), 6.91 (d, J = 7.7 Hz, 1H), |
| | 6.39-5.97 (m, 1H), 3.79-3.62 (m, |
| | 2H). |
| 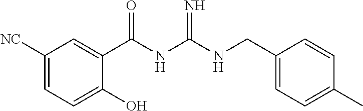 [SMILES: Cc1ccc(CNC(=N)NC(=O)c2cc(C#N)ccc2O)cc1] | 5-cyano-2-hydroxy-N-(N-(4- methylbenzyl)carbamimidoyl)benzamide LC-MS: m/z 309 (M + H)⁺. 1H NMR (400 MHz, DMSO-d6) δ 16.07 (s, 1H), 9.66 (s, 1H), 8.82 (s, 1H), 8.11 (s, 1H), 7.81 (s, 1H), 7.67 (d, J = 8.6 Hz, 1H), 7.22 (dd, J = 14.5 Hz, 4H), |
| | 6.89 (d, J = 8.6 Hz, 1H), 4.41 (d, J = 4.0 |
| | Hz, 2H), 2.29 (s, 3H). |
| 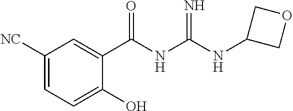 [SMILES: N#Cc1ccc(O)c(C(=O)NC(=N)NC2COC2)c1] | 5-cyano-2-hydroxy-N-(N-oxetan-3- ylcarbamimidoyl)benzamide LC-MS: m/z 261 (M + H)⁺. 1H NMR (400 MHz, DMSO-d6) δ 15.84 (s, 1H), 8.14 (d, J = 2.2 Hz, 1H), 7.70 (d, J = 7.5 Hz, 1H), 6.92 (d, J = 8.6 Hz, 1H), 4.85-4.79 (m, 2H), 4.79- |
| | 4.72 (m, 1H), 4.52 (s, 2H). |
| 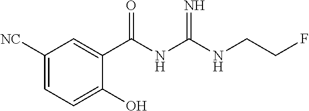 [SMILES: N#Cc1ccc(O)c(C(=O)NC(=N)NCCF)c1] | 5-cyano-N-(N-(2- fluoroethyl)carbamimidoyl)-2- hydroxybenzamide LC-MS: m/z 251 (M + H)⁺. 1H NMR (400 MHz, DMSO-d6) δ 16.03 (s, 1H), 8.13 (s, 1H), 7.68 (d, J = 8.4 Hz, 1H), 6.90 (d, J = 8.7 Hz, 1H), |
| | 4.63 (t, J = 4.8 Hz, 1H), 4.51 (t, J = 4.8 |
| | Hz, 1H), 3.59 (t, J = 4.9 Hz, 1H), 3.52 |
| | (t, J = 4.9 Hz, 1H). |
| 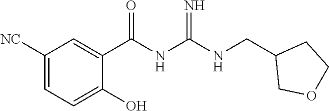 [SMILES: N#Cc1ccc(O)c(C(=O)NC(=N)NCC2CCOC2)c1] | 5-cyano-2-hydroxy-N-(N- ((tetrahydrofuran-3- yl)methyl)carbamimidoyl)benzamide LC-MS: m/z 289 (M + H)⁺. 1H NMR (400 MHz, DMSO-d6) δ 16.13 (s, 1H), 8.12 (s, 1H), 7.66 (d, J = 8.4 Hz, 1H), 6.90 (d, J = 8.7 Hz, 1H), |
| | 3.79-3.70 (m, 2H), 3.66-3.60 (m, |
| | 1H), 3.43-3.40 (m, 1H), 3.24-3.20(m, |
| | 2H), 2.50-2.46 (m, 1H), 2.03-1.95 |
| | (m, 1H), 1.61-1.52 (m, 1H). |
| 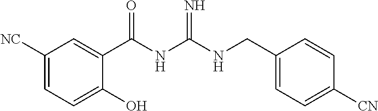 [SMILES: N#Cc1ccc(CNC(=N)NC(=O)c2cc(C#N)ccc2O)cc1] | 5-cyano-N-(N-(4- cyanobenzyl)carbamimidoyl)-2- hydroxybenzamide LC-MS: m/z 320 (M + H)⁺. 1H NMR (400 MHz, DMSO-d6) δ 15.56 (s, 1H), 8.50 (br s, 2H), 8.09 (s, 1H), 7.84 (d, J = 8.2 Hz, 2H), 7.58 (d, J = |
| | 8.6 Hz, 1H), 7.53 (d, J = 8.3 Hz, 2H), |
| | 6.78 (d, J = 8.5 Hz, 1H), 4.52 (s, 2H). |
| 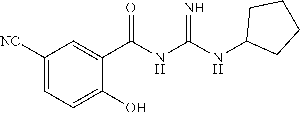 [SMILES: N#Cc1ccc(O)c(C(=O)NC(=N)NC2CCCC2)c1] | 5-cyano-N-(N- cyclopentylcarbamimidoyl)-2- hydroxybenzamide LC-MS: m/z 287 (M + H)⁺. 1H NMR (400 MHz, DMSO-d6) δ 16.11 (s, 1H), 9.28 (d, J = 7.1 Hz, 1H), 8.84 (s, 1H), 8.11 (s, 1H), 7.79 (s, 1H), |
| | 7.65 (d, J = 8.4 Hz, 1H), 6.87 (d, J = 8.5 |
| | Hz, 1H), 4.03-3.84 (m, 1H), 2.06- |
| | 1.89 (m, 2H), 1.77-1.64 (m, 2H), 1.62- |
| | 1.43 (m, 4H). |
| 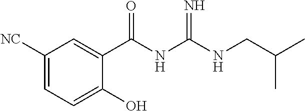 [SMILES: CC(C)CNC(=N)NC(=O)c1cc(C#N)ccc1O] | 5-cyano-2-hydroxy-N-(N- isobutylcarbamimidoyl)benzamide LC-MS: m/z 261 (M + H)⁺. 1H NMR (400 MHz, DMSO-d6) δ 16.20 (s, 1H), 9.43 (s, 1H), 8.12 (d, J = 2.0 Hz, 1H), 7.65 (dd, J = 8.7, 2.1 Hz, 1H), 6.87 (d, J = 8.6 Hz, 1H), 3.04 (t, J = |
| | 6.4 Hz, 2H), 1.90-1.79 (m, 1H), |
| | 0.92 (d, J = 6.7 hz, 6H). |

### Synthesis of A92

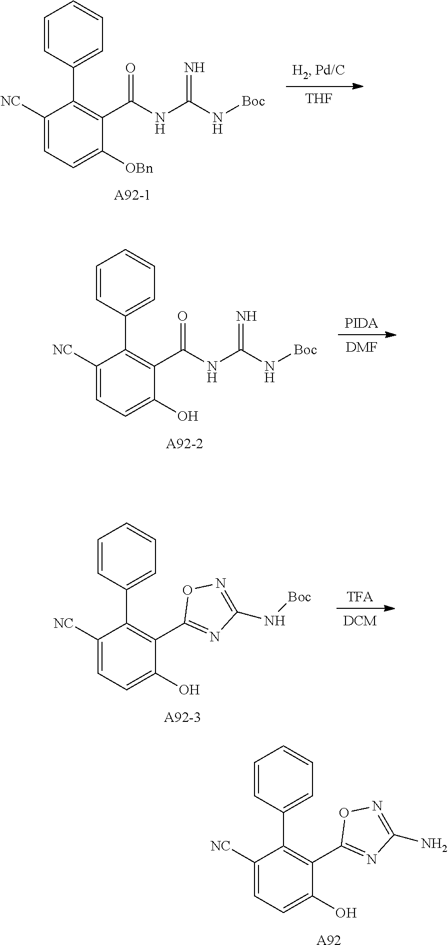

Intermediate A92-1 was prepared following general procedures 5 and 7.

### Step 1: Synthesis of Compound A92-2

10% Pd/C (42 mg) was added under N₂ atmosphere to a solution of A92-1 (141 mg, 0.3 mmol) in THF (8 mL), and the resulting mixture was stirred at room temperature for 30 minutes under 15 psi H₂. The mixture was then filtered and the filtrate was concentrated under vacuum to give A92-2 (98 mg, 86% yield) as a white solid without any further purification. LC-MS: m/z 381 (M+H)⁺.

### Step 2: Synthesis of Compound A92-3

(diacetoxyiodo)benzene (166 mg, 0.52 mmol) was added at 0° C. under N₂ atmosphere to a solution of A92-2 (98 mg, 0.26 mmol) in DMF (8 mL), and the resulting mixture was stirred at room temperature for 6 hours. The mixture was then diluted with EtOAc and washed with saturated aq. NH₄Cl solution. The organic layer was then dried over Na₂SO₄, filtered and concentrated under vacuum. The residue was purified by column chromatography on silica gel (eluted with Petroleum Ether:EtOAc=50:1 to 4:1) to give compound A92-3 (52 mg, 53% yield) as a yellow oil. LC/MS (ESI) m/z: 379 (M+H)⁺.

### Step 3: Synthesis of Compound A92

TFA (1 mL) was added dropwise at 0° C. under N₂ atmosphere to a solution of compound A92-3 (52 mg, 0.14 mmol) in anhydrous DCM (2 mL), and the resulting mixture was stirred at room temperature for 3 hours. The mixture was then concentrated under vacuum and the residue was purified via prep-HPLC (C18, 0% to 50% acetonitrile in H₂O with 0.1% NH₃.H₂O) to give compound A92 (13 mg, 33% yield) as a white solid. LC/MS (ESI) m/z: 279 (M+H)⁺. 1H NMR (400 MHz, DMSO-d6) δ 7.70 (d, J=8.6 Hz, 1H), 7.40-7.31 (m, 3H), 7.26-7.16 (m, 2H), 6.83 (d, J=8.1 Hz, 1H), 6.06 (s, 2H).

### Synthesis of A133

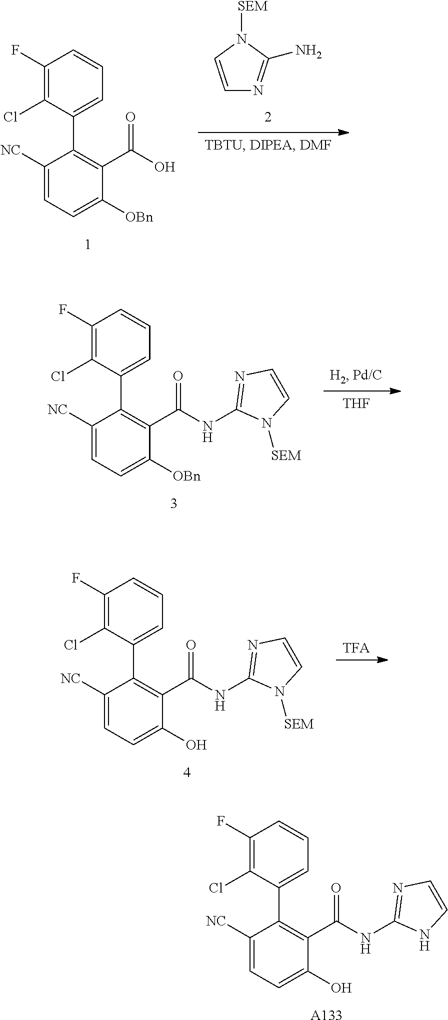

### Step 1: Synthesis of Compound A133-3

DIPEA (108 mg, 0.84 mmol) and TBTU (81 mg, 0.25 mmol) were added at room temperature to a mixture of compound A133-1 (80 mg, 0.21 mmol) and A133-2 (54 mg, 0.25 mmol) in DMF (3 mL), and the resulting mixture was stirred at 30° C. for 16 hours. The mixture was then diluted with water and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=8:1 to 3:1) to give compound A133-3 (30 mg, 25% yield) as a light yellow solid. LC/MS (ESI) m/z: 577 (M+H)⁺.

### Step 2: Synthesis of Compound A133-4

10% Pd/C (30 mg) was added at 0° C. to a solution of compound A133-3 (30 mg, 0.05 mmol) in THF (4 mL), and the mixture was degassed under N₂ atmosphere for three times and stirred under a H₂ at room temperature for 30 minutes. The mixture was then filtered and the filtrate was concentrated under vacuum. The residue was purified by column chromatography on silica gel (eluted with Petroleum Ether:EtOAc=10:1 to 4:1) to give compound A133-4 (15 mg, 59% yield) as a white solid. LC/MS (ESI) m/z: 487 (M+H)⁺.

### Step 3: Synthesis of Compound A133

Compound A133-4 (15 mg, 0.03 mmol) was dissolved in TFA (2 mL) and reaction stirred at room temperature for 1 hour. The reaction mixture was then concentrated under vacuum and the residue was dissolved in saturated aq. NaHCO₃ solution and extracted with EtOAc twice. The combined organic layers were washed with brine, dried over anhydrous Na₂SO₄, filtered and concentrated under vacuum. The residue was purified by prep-HPLC (C18, 0% to 50% acetonitrile in H₂O with 0.1% NH₃.H₂O) to give compound A133 (3 mg, 27% yield) as a white solid. LC/MS (ESI) m/z: 357 (M+H)⁺. ¹H NMR (400 MHz, DMSO-d6) δ 12.35 (br s, 2H), 7.72 (d, J=8.8 Hz, 1H), 7.44-7.32 (m, 2H), 7.10 (d, J=6.4 Hz, 1H), 7.00 (d, J=8.4 Hz, 1H), 6.87 (s, 2H).

### Synthesis of A141

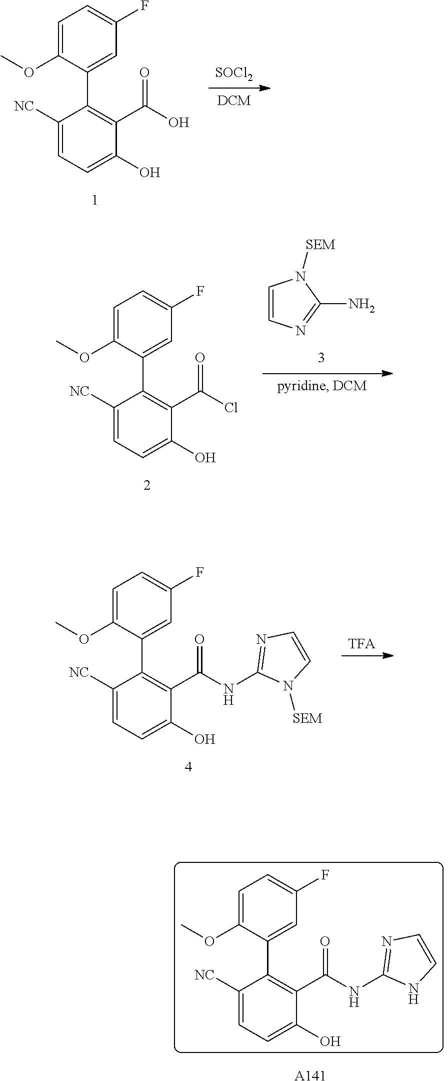

### Step 1: Synthesis of Compound A141-2

Thionyl chloride (0.51 mL, 7.0 mmol) was added at 0° C. to a solution of compound A141-1 (200 mg, 0.70 mmol) in DCM (5 mL), and the resulting mixture was stirred at 65° C. for 2 hours. The mixture was then concentrated under vacuum to give compound A141-2 (200 mg, 99% yield) as a light yellow solid which was used in the next step without any further purification.

### Step 2: Synthesis of Compound A141-4

Anhydrous pyridine (0.28 mL, 3.48 mmol) was added at 0° C. under N₂ atmosphere to a solution of A141-3 (446 mg, 2.09 mmol) in anhydrous DCM (5 mL), and the reaction was stirred at 0° C. for 30 minutes. A solution of A141-2 (200 mg, 0.66 mmol) in DCM (5 mL) was added dropwise to the above reaction and the resulting mixture was stirred at room temperature overnight under N₂ atmosphere. The mixture was then concentrated under vacuum and the residue was purified by column chromatography on silica gel (eluted with Petroleum Ether:EtOAc=30:1 to 2:1) to give A141-4 (60 mg, 18%) as a yellow oil. LC/MS (ESI) m/z: 483 (M+H)⁺.

### Step 3: Synthesis of Compound A141

A141-4 (60 mg, 0.12 mmol) was dissolved at 0° C. in TFA (2 mL), and the reaction was stirred at 40° C. for 3 hours. The mixture was then concentrated under vacuum and the residue was dissolved in EtOAc, basified with saturated aq. NaHCO₃ solution and extracted with EtOAc twice. The combined organic layers were washed with brine, dried over anhydrous Na₂SO₄, filtered and concentrated under vacuum. The residue was purified via prep-HPLC (C18, 0% to 50% acetonitrile in H₂O with 0.1% NH₃.H₂O) to give A141 (15 mg, 34%) as a white solid. LC/MS (ESI) m/z: 353 (M+H)⁺. ¹H NMR (400 MHz, DMSO-d6) δ 12.33 (br s, 2H), 7.68 (d, J=8.8 Hz, 1H), 7.15-7.09 (m, 1H), 7.03 (dd, J=8.8, 4.4 Hz, 1H), 6.99-6.92 (m, 2H), 6.85 (s, 2H), 3.65 (s, 3H).

### Synthesis of A156

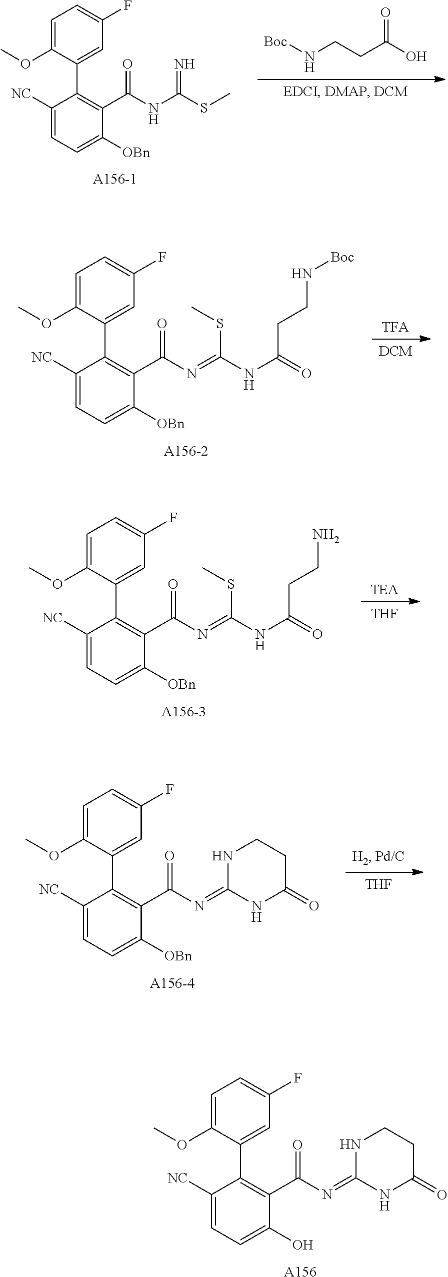

### Step 1: Synthesis of Compound A156-2

EDCI (104 mg, 0.67 mmol) and DMAP (163 mg, 1.33 mmol) were added to a mixture of compound A156-1 (200 mg, 0.45 mmol) and Boc-beta-alanine (84 mg, 0.45 mmol) in DCM (10 mL), and the resulting mixture was stirred at 35° C. for 24 hours. The mixture was then diluted with water and extracted with DCM twice. The combined organic layers were washed with brine, dried over anhydrous Na₂SO₄, filtered and concentrated under vacuum. The residue was purified by flash column chromatography on silica gel (Petroleum Ether:EtOAc=10:1 to 2:1) to give compound A156-2 (210 mg, 76% yield) as a light yellow solid. LC/MS (ESI) m/z: 621 (M+H)⁺.

### Step 2: Synthesis of Compound A156-3

TFA (3 mL) was added at 0° C. to a solution of compound A156-2 (210 mg, 0.34 mmol) in DCM (3 mL) and the reaction was stirred at room temperature for 1 hour. The mixture was then concentrated under vacuum to give compound A156-3 (176 mg, 99% yield) as a light yellow solid without any further purification. LC/MS (ESI) m/z: 521 (M+H)⁺.

### Step 3: Synthesis of Compound A156-4

TEA (0.19 mL, 1.39 mmol) was added to a solution of compound A156-3 (176 mg, 0.35 mmol) in THF (5 mL) and the reaction mixture was stirred at room temperature for 30 minutes. The mixture was then concentrated under vacuum and the residue was purified by flash column chromatography on silica gel (DCM:MeOH=200:1 to 100:1) to give compound A156-4 (84 mg, 51% yield) as a white solid. LC/MS (ESI) m/z: 473 (M+H)⁺.

### Step 4: Synthesis of Compound A156

10% Pd/C (60 mg) was added to a solution of compound A156-4 (60 mg, 0.15 mmol) in THF (5 mL) and the reaction mixture was degassed under N₂ atmosphere for three times and stirred under H₂ at room temperature for 30 minutes. The mixture was then filtered and the filtrate was concentrated under vacuum. The residue was purified via prep-HPLC (C18, 0% to 50% acetonitrile in H₂O with 0.1% NH₃.H₂O) to give compound A156 (16 mg, 24% yield) as a white solid. LC/MS (ESI) m/z: 383 (M+H)⁺. ¹H NMR (400 MHz, DMSO-d6) δ 14.71 (s, 1H), 11.31 (s, 1H), 10.18 (s, 1H), 7.73 (d, J=8.7 Hz, 1H), 7.12 (td, J=8.7, 3.1 Hz, 1H), 7.05-6.97 (m, 2H), 6.92 (dd, J=8.8, 3.1 Hz, 1H), 3.65 (s, 3H), 3.53 (t, J=7.2 Hz, 2H), 2.59 (t, J=7.2 Hz, 2H).

### Synthesis of A162 and A163

### Synthesis of Key Intermediate 7

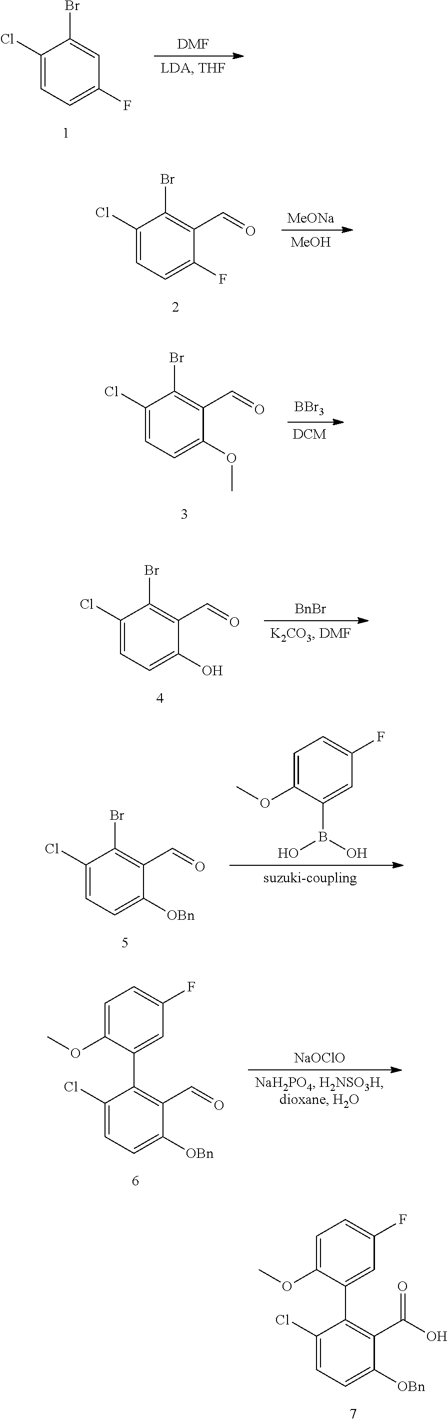

### Step 1: Synthesis of Compound 2

LDA (4.3 mL, 2.0 M in THF) was added under N₂ atmosphere at −78° C. to a solution of 1 (1.5 g, 7.2 mmol) in anhydrous THF (30 mL), and the reaction mixture was stirred at −78° C. for 2 hours before anhydrous DMF (0.6 g, 8.66 mmol) was added. The reaction was stirred −78° C. for an additional hour, and then the reaction was quenched with iced saturated aq. NH₄Cl solution and extracted with EtOAc twice. The combined organic layers were washed with brine, dried over Na₂SO₄, filtered and concentrated under vacuum. The residue was purified by flash column chromatography on silica gel (eluted with Petroleum Ether:EtOAc=20:1 to 5:1) to give 2 (1.4 g, 83% yield). LC/MS (ESI) m/z: 237 (M+H)⁺.

### Step 2: Synthesis of Compound 3

Sodium Methoxide (1.27 g, 23.59 Mmol) was Added to a Solution of Compound 2 (1.4 g, 5.9 mmol) in MeOH (5 mL). The resulting mixture was heated to 80° C. for 30 minutes, then the mixture was concentrated under vacuum. The residue was diluted with water (100 mL) and extracted with EtOAc. The organic layer was dried over anhydrous Na₂SO₄, filtered and concentrated under vacuum to give compound 3 (1.23 g, 84% yield) as a yellow solid and was used without any further purification. LC/MS (ESI) m/z: 249 (M+H)⁺.
Step 3: Synthesis of compound 4

Boron tribromide (26.75 mL, 48.1 mmol) was slowly added at −20° C. under N₂ atmosphere to a solution of compound 3 (1.2 g, 4.8 mmol) in dry DCM (20 mL). The resulting mixture was slowly warmed to room temperature during a period of 1 hour, and then the mixture was quenched by addition of MeOH (0.4 mL) at 0° C. and concentrated under vacuum. The residue was purified by flash column chromatography on silica gel (eluted with 0% to 20% EtOAc in Petroleum Ether) to give compound 4 (1.05 g, 93% yield) as a light yellow solid. LC/MS (ESI) m/z: 235 (M+H)⁺.

### Step 4: Synthesis of Compound 5

Benzyl Bromide (0.59 mL, 4.9 Mmol) and Potassium Carbonate (0.76 mL, 13.38 Mmol) were Added to a solution of compound 4 (1.05 g, 4.46 mmol) in DMF (15 mL). The resulting mixture was stirred at overnight at room temperature. The reaction was then quenched by the addition of water (80 mL) and extracted with EtOAc twice. The combined organic layers were washed with brine, dried over anhydrous Na₂SO₄, filtered and concentrated under vacuum. The residue was purified by column chromatography on silica gel (eluted with Petroleum Ether:EtOAc=8:1) to give compound 5 (1.26 g, 87% yield) as a light yellow solid. LC/MS (ESI) m/z: 325 (M+H)⁺.
Step 5: Synthesis of compound 6

Potassium carbonate (637 mg, 4.61 mmol) was added under N₂ atmosphere to a mixture of compound 5 (500 mg, 1.54 mmol) and (5-fluoro-2-methoxyphenyl)boronic acid (392 mg, 2.3 mmol) in dioxane (25 mL) and H₂O (2.5 mL) followed by the addition of Pd(PPh₃)₄ (355 mg, 0.31 mmol). The resulting mixture was stirred at 95° C. for 15 hours under N₂ atmosphere. The reaction was then cooled to room temperature, diluted with EtOAc and washed with water. The organic layer was dried over Na₂SO₄, filtered and concentrated under vacuum. The residue was purified by flash column chromatography on silica gel (eluted with Petroleum Ether:EtOAc=20:1 to 5:1) to give compound 6 (520 mg, 91% yield). LC/MS (ESI) m/z: 371 (M+H)⁺.

### Step 6: Synthesis of Compound 7

A solution of compound 6 (520 mg, 1.48 mmol) in dioxane (30 mL) was added to a mixture of NaH₂PO₄ (712 mg, 5.9 mmol) and sulfamic acid (624 mg, 2.23 mmol) in water (20 mL), followed by the addition of a solution of sodium chlorite (175 mg, 1.93 mmol) in H₂O (10 mL). The resulting mixture was stirred at 0° C. for 2 hour, and then the mixture was diluted with water and extracted with EtOAc twice. The combined organic layers were dried over anhydrous Na₂SO₄, filtered and concentrated under vacuum. The residue was purified by column chromatography on silica gel (eluted with Petroleum Ether:EtOAc=10:1 to 1:1) to give compound 7 (480 mg, 84% yield) as a light yellow solid. LC/MS (ESI) m/z: 385 (M−H)⁺.

### Procedure for the Preparation of A162

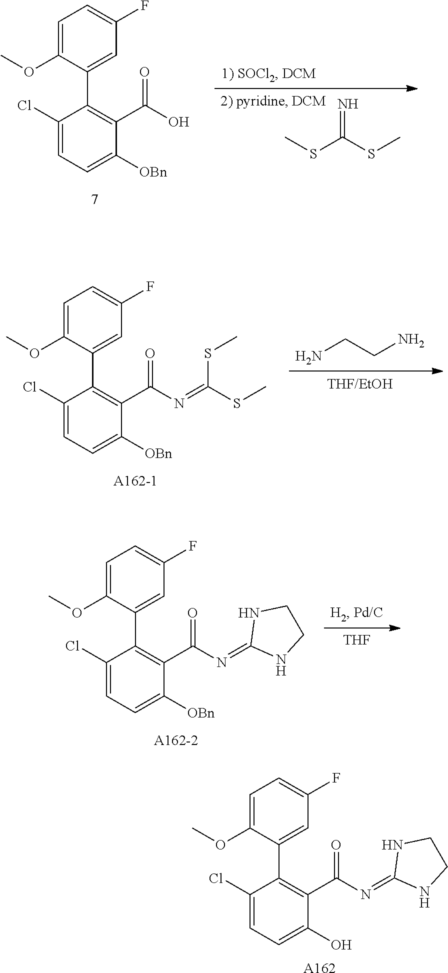

### Step 1: Synthesis of Compound A162-1

Thionyl chloride (0.3 mL, 3.9 mmol) was added to a solution of compound 7 (150 mg, 0.39 mmol) in anhydrous DCM (10 mL), and the resulting mixture was heated to 65° C. for 2 hours. The mixture was then cooled to room temperature and concentrated under vacuum. The crude acyl chloride was dissolved in anhydrous DCM (3 mL) and added dropwise at 0° C. under N₂ atmosphere to a solution of bis(methylsulfanyl)methanimine (71 mg, 0.58 mmol) and dry pyridine (1.03 g, 3.878 mmol) in anhydrous DCM (10 mL). The resulting mixture was stirred at 20° C. for 45 minutes, and then the mixture was quenched with saturated aq. NaHCO₃ solution and extracted with DCM. The organic layer was dried over anhydrous Na₂SO₄, filtered and concentrated under vacuum. The residue was purified by column chromatography on silica gel (eluted with Petroleum Ether:EtOAc=10:1 to 1:1) to give compound A162-1 (154 mg, 81% yield) as a light yellow solid. LC/MS (ESI) m/z: 490 (M+H)⁺.

Step 2: Synthesis of compound A162-2

Ethane-1,2-diamine (0.09 mL, 1.23 mmol) was added to a solution of A162-1 (150 mg, 0.31 mmol) in THF (3 mL) and EtOH (3 mL). The reaction mixture was heated to 80° C. for 1 hour. The mixture was then concentrated under vacuum and the residue was recrystallized from MTBE (4 mL) to give A162-2 (110 mg, 79%) as a white solid. LC/MS (ESI) m/z: 454 (M+H)⁺.

Step 3: Synthesis of compound A162

10% Pd/C (50 mg) was added under N₂ atmosphere to a solution of A162-2 (100 mg, 0.22 mmol) in THF (8 mL), and the resulting mixture was stirred at room temperature for 25 minutes under 15 psi H₂. The mixture was then filtered and the filtrate was concentrated under vacuum. The residue was purified via prep-HPLC (C18, 0% to 50% acetonitrile in H₂O with 0.1% NH₃. E120) to give A162 (25 mg, 31% yield) as a white solid. LC/MS (ESI) m/z: 364 (M+H)⁺. 1H NMR (400 MHz, DMSO-d6) δ:15.43 (s, 1H), 8.34 (s, 2H), 7.37 (d, J=8.8 Hz, 1H), 7.03 (td, J=8.7, 3.1 Hz, 1H), 6.95 (dd, J=9.0, 4.6 Hz, 1H), 6.86 (d, J=8.8 Hz, 1H), 6.71 (dd, J=9.0, 3.1 Hz, 1H), 3.61 (s, 3H), 3.50 (s, 4H).

### Procedure for the Preparation of A163

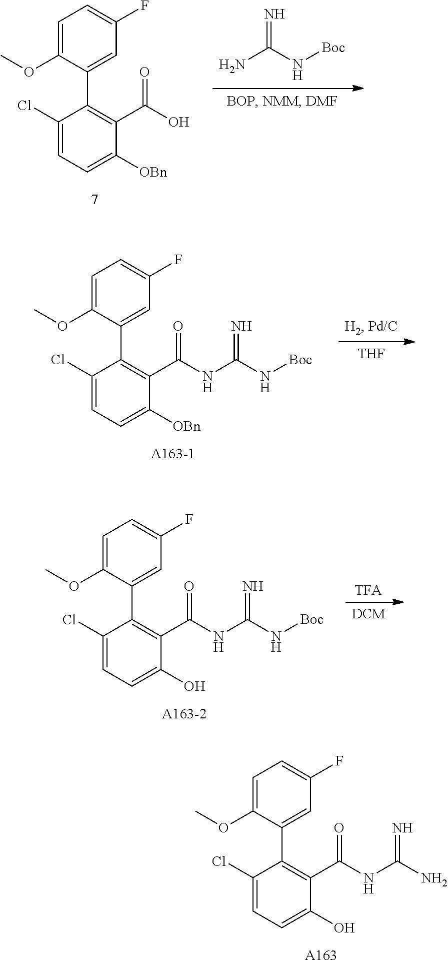

### Step 1: Synthesis of Compound A163-1

NMM (0.17 mL, 1.55 mmol) and PyBOP (161 mg, 0.362 mmol) were added under N₂ atmosphere to a solution of compound 7 (100 mg, 0.26 mmol) and tert-butyl N-carbamimidoylcarbamate (107 mg, 0.67 mmol) in DMF (12 mL). The resulting mixture was stirred at room temperature for 15 hours. The mixture was then poured into water and extracted with EtOAc twice. The combined organic layers were washed with brine, dried over Na₂SO₄, filtered and concentrated under vacuum. The residue was purified by flash column chromatography on silica gel (eluted with Petroleum Ether:EtOAc=10:1 to 3:1) to give A163-1 (81 mg, 59% yield) as a white solid. LC/MS (ESI) m/z: 528 (M+H)⁺.

### Step 2: Synthesis of Compound A163-2

10% Pd/C (35 mg) was added under N₂ atmosphere to a solution of A163-1 (70 mg, 0.133 mmol) in THF (8 mL) and the resulting mixture was stirred at room temperature for 1 hour under 15 psi H₂. The mixture was then filtered and the filtrate was concentrated under vacuum to give A163-2 (51 mg, 88% yield) as a white solid without any further purification. LC/MS (ESI) m/z: 438 (M+H)⁺.

### Step 3: Synthesis of Compound A163

TFA (3 mL) was added at 0° C. to a solution of A163-2 (50 mg, 0.12 mmol) in DCM (3 mL). The resulting mixture was stirred at room temperature for 1 hour and then the mixture was concentrated under vacuum. The residue was purified via prep-HPLC (C18, 0% to 50% acetonitrile in H₂O with 0.1% NH₃.H₂O) to give A163 (12 mg, 31% yield) as a white solid. LC-MS (ESI) m/z 338 (M+H)⁺. ¹H NMR (400 MHz, DMSO-d6) δ:15.75 (s, 1H), 8.09 (br s, 2H), 7.36 (d, J=8.8 Hz, 1H), 7.03 (td, J=8.7, 3.1 Hz, 1H), 6.94 (dd, J=9.0, 4.7 Hz, 1H), 6.83 (d, J=8.8 Hz, 1H), 6.71 (dd, J=9.0, 3.1 Hz, 1H), 3.61 (s, 3H).

### Synthesis of A161

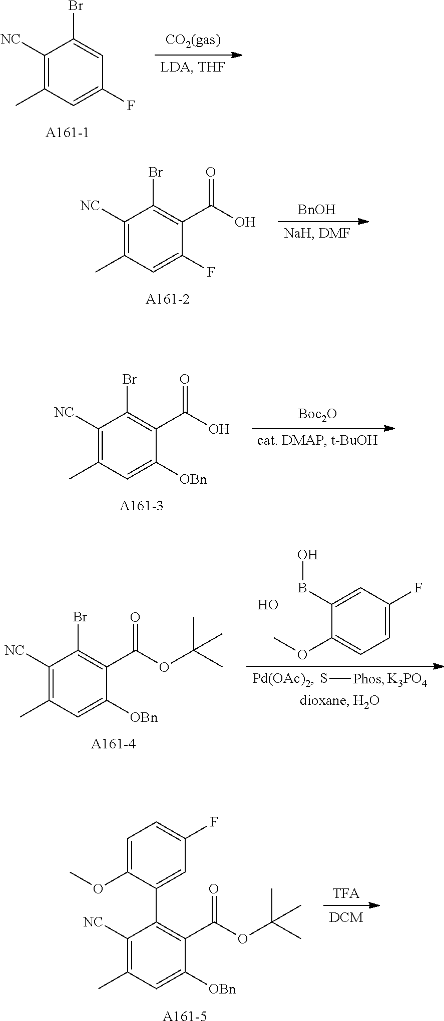

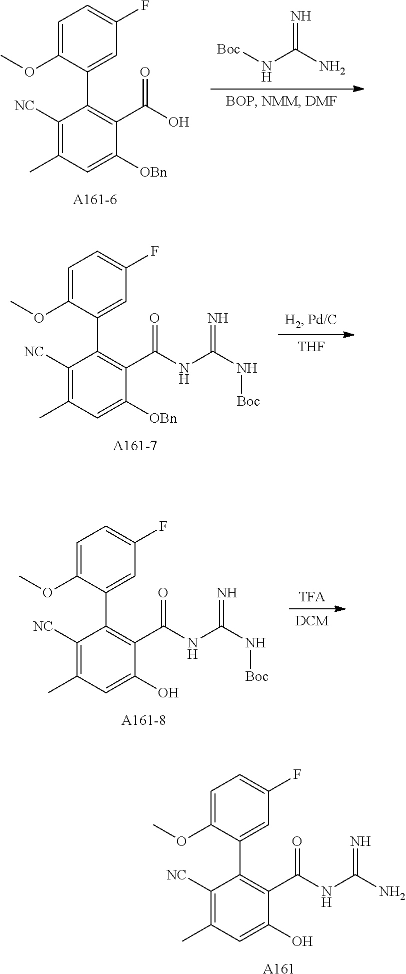

### Step 1: Synthesis of Compound A161-2

LDA (0.3 mL, 2M in THF) was added at −78° C. under N₂ atmosphere to a solution of compound A161-1 (100 mg, 0.47 mmol) in anhydrous THF (10 mL), and the resulting mixture was stirred at −78° C. for 1 hour. Then CO₂ (gas) was bubbled into the above mixture at −78° C. for 30 minutes. The mixture was quenched with ice/water and extracted with methyl tert-butyl ether twice. Then the aqueous layer was separated, acidified with 0.5 M aq. HCl solution and extracted with EtOAc twice. The combined organic layers were washed with brine, dried over Na₂SO₄, filtered and concentrated under vacuum to give compound A161-2 (80 mg, 66% yield) as a yellow solid. LC/MS (ESI) m/z: 256 (M−H)⁻.

### Step 2: Synthesis of Compound A161-3

60% NaH (26 mg, 0.65 mmol) was added at 0° C. under N₂ atmosphere to a solution of benzyl alcohol (0.05 mL, 0.47 mmol) in anhydrous DMF (5 mL). The mixture was stirred at 0° C. for 1 hour before A161-2 (80 mg, 0.31 mmol) was added. The resulting mixture was stirred at room temperature for 1.5 hours. The mixture was then quenched with ice/H₂O and extracted with Methyl tert-butyl ether twice. The aqueous layer was separated, acidified with 0.5 M aq. HCl solution and extracted with EtOAc twice. The combined organic layers were washed with brine, dried over anhydrous Na₂SO₄, filtered and concentrated under vacuum. The residue was purified by column chromatography on silica gel (eluted with DCM:MeOH=80:1 to 40:1) to give compound A161-3 (100 mg, 93% yield) as a light yellow solid. LC/MS (ESI) m/z: 344 (M−H)⁻.

### Step 3: Synthesis of Compound A161-4

di-tert-butyl dicarbonate (0.12 mL, 0.58 mmol) and DMAP (7 mg, 0.06 mmol) were added to a solution of compound A161-3 (100 mg, 0.29 mmol) in t-BuOH (10 mL), and the mixture was stirred at 60° C. for 16 hours. The mixture was then cooled to room temperature and concentrated under vacuum. The residue was purified by column chromatography on silica gel (Petroleum Ether:EtOAc=50:1 to 10:1) to give compound A161-4 (80 mg, 69% yield) as a white solid. LC/MS (ESI) m/z: 402 (M+H)⁺.

### Step 4: Synthesis of Compound A161-5

K₃PO₄ (106 mg, 0.50 mmol) and S-Phos (16 mg, 0.04 mmol) were added under N₂ atmosphere to a mixture of compound A161-4 (80 mg, 0.2 mmol) and 5-fluoro-2-methoxyphenylboronic acid (54 mg, 0.32 mmol) in dioxane (8 mL) and H₂O (1 mL) followed by the addition of Pd(OAc)₂ (9 mg, 0.04 mmol). The resulting mixture was stirred at 95° C. for 16 hours under N₂ atmosphere. The mixture was then cooled to room temperature, diluted with EtOAc and washed with water. The organic layer was then dried over Na₂SO₄, filtered and concentrated under vacuum. The residue was purified by flash column chromatography on silica gel (eluted with Petroleum Ether:EtOAc=10:1 to 5:1) to give compound A161-5 (70 mg, 79% yield) as a white solid. LC/MS (ESI) m/z: 392 (M-56+H)⁺.

### Step 5: Synthesis of Compound A161-6

TFA (2 mL) was added at 0° C. to a solution of compound A161-5 (70 mg, 0.16 mmol) in DCM (4 mL) and the reaction was stirred at room temperature for 1 hour. The mixture was then concentrated under vacuum to give compound A161-6 (60 mg, 98% yield) as a light yellow solid without any further purification. LC/MS (ESI) m/z: 390 (M−H)⁻.

### Step 6: Synthesis of Compound A161-7

4-Methylmorpholine (0.05 mL, 0.5 mmol) and PyBOP (47 mg, 0.11 mmol) were added to a mixture of compound A161-6 (30 mg, 0.08 mmol) and Boc-guanidine (230 mg, 0.52 mmol) in DMF (2 mL), and the reaction was stirred at room temperature for 16 hours. The mixture was then diluted with H₂O and extracted with EtOAc twice. The combined organic layers were washed with saturated aq. NH₄Cl solution and brine, dried over anhydrous Na₂SO₄, filtered and concentrated under vacuum. The residue was purified by column chromatography on silica gel (Petroleum Ether:EtOAc=10:1 to 2:1) to give compound A161-7 (40 mg, 98% yield) as a white solid. LC/MS (ESI) m/z: 533 (M+H)⁺.

### Step 7: Synthesis of Compound A161-8

10% Pd/C (20 mg) was added under N₂ atmosphere to a solution of compound A161-7 (40 mg, 0.08 mmol) in THF (4 mL) and the resulting mixture was degassed under N₂ atmosphere for three times and stirred under H₂ at room temperature for 30 minutes. The mixture was then filtered and the filtrate was concentrated under vacuum to give compound A161-8 (30 mg, 90% yield) as a white solid without any further purification. LC/MS (ESI) m/z: 443 (M+H)⁺.

### Step 8: Synthesis of Compound A161

TFA (3 mL) was added at 0° C. to a solution of compound A161-8 (30 mg, 0.07 mmol) in DCM (3 mL) and the reaction was stirred at room temperature for 1 hour. The mixture was then concentrated under vacuum and the residue was purified via prep-HPLC (C18, 0% to 50% acetonitrile in H₂O with 0.1% NH₃.H₂O) to give compound A161 (7.2 mg, 31% yield) as a white solid. LC/MS (ESI) m/z: 343 (M+H)⁺. ¹H NMR (400 MHz, DMSO-d6) δ 7.67 (br s, 3H), 7.09 (td, J=8.7, 3.1 Hz, 1H), 6.99 (dd, J=9.0, 4.6 Hz, 1H), 6.85 (dd, J=8.9, 3.1 Hz, 1H), 6.77 (s, 1H), 3.64 (s, 3H), 2.36 (s, 3H).

### Example 4: General Procedures for the Synthesis of Representative Compounds of the Invention

### General Procedure A

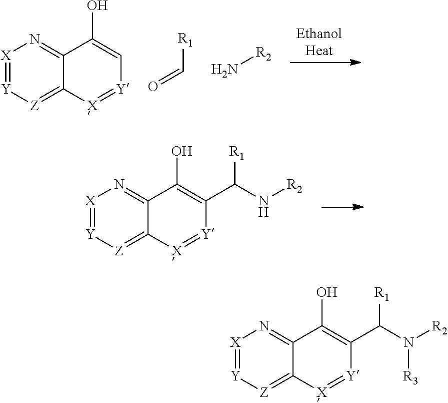

### Step 1

The appropriate aldehyde (1 equiv.) and amine (1.1 equiv.) were dissolved in Ethanol (0.3M) and the reaction was stirred at RT for 10-15 min before the appropriate quinoline (1 equiv.) was added and the reaction was stirred overnight at 60-80° C. In some case microwave-assisted synthesis was used. The reaction mixture was concentrated under vacuum and the crude was purified by prep HPLC.

### Step 2

Depending on the nature of R₃, different known reaction conditions were used to install R₃

### General Procedure B

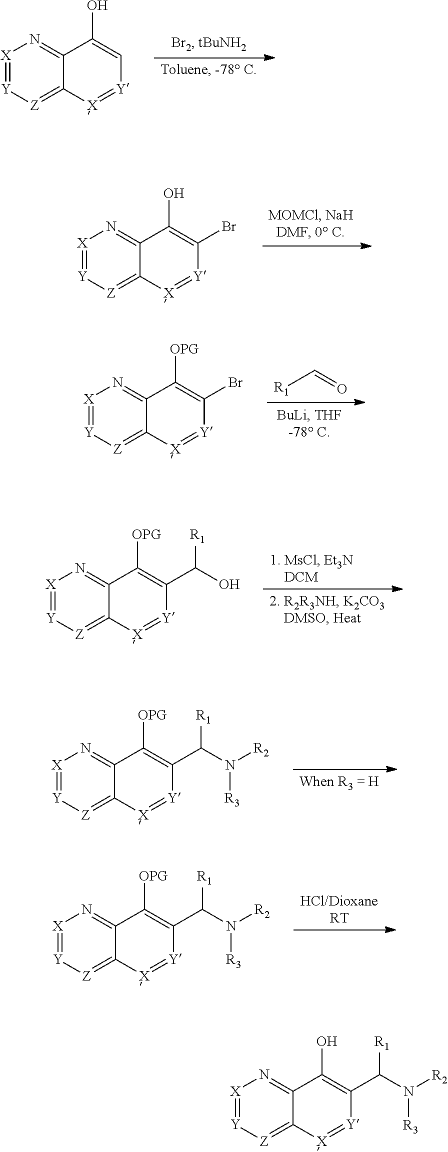

### Step 1

To a solution of t-BuNH₂ (2 equiv.) in toluene (0.1M) was added Br₂ (1 equiv.) dropwise at −78° C., and the mixture was stirred for 10 min. A solution of the appropriate quinoline (1 equiv.) in CHCl₃ (3M) was added dropwise to the above mixture at −78° C., and the resulting mixture was stirred at −78° C. for 1 hr. The reaction was quenched with saturated aq. NaHCO₃ solution and the aqueous layer was extracted with EtOAc (×3). The combined organic layers were washed with brine, dried over anhydrous Na₂SO₄, filtered and concentrated under vacuum. The residue was purified by flash chromatography.

### Step 2

To a solution of the above intermediate (1 equiv.) in anhydrous DMF (0.2M) was added NaH (1.2 eq, 60% in mineral oil) portion wise followed by dropwise addition of MOMCl (0.95 equiv.) at 0° C. The reaction mixture was stirred at 0° C. until full conversion. The mixture was then poured into ice-water and the aqueous layer extracted with EtOAc (×3). The combined organic layers were washed with water and brine, dried over anhydrous Na₂SO₄, filtered and concentrated under vacuum. The residue was purified by flash chromatography.

### Step 3

To a solution of the above intermediate (1 equiv.) in anhydrous THF (0.2M) was added n-BuLi (1.6 mol/L. 1.05 equiv.) dropwise at −78° C. under N₂ atmosphere. The reaction was stirred at this temperature for 30 min, then the appropriate aldehyde (1.5 equiv.) was added dropwise and the resulting mixture was stirred at −78° C. until full conversion. The reaction was quenched with saturated aq. NH₄Cl solution and the aqueous layer was extracted with EtOAc (×3). The combined organic layers were washed with brine, dried over anhydrous Na₂SO₄, filtered and concentrated under vacuum. The residue was purified by flash chromatography.

### Step 4

To a mixture of the above intermediate (1 equiv.) and TEA (3 equiv.) in DCM (0.1M) was added MsCl (1.2 equiv.) dropwise at 0° C. and the resulting mixture was stirred at RT until full conversion. The reaction was quenched with saturated aq. NH₄Cl solution and the aqueous layer was extracted with EtOAc (×2). The combined organic layers were washed with brine, dried over anhydrous Na₂SO₄, filtered and concentrated under vacuum. The crude was used for next step without any further purification.

### Step 5

To a solution of the mesylate intermediate (1 equiv.) in DMSO, K₂CO₃ (1.5 equiv.) was added followed by the appropriate amine (2 equiv.) and the resulting mixture was stirred at 40-50° C. until full conversion. The mixture was diluted with DCM and washed with water and brine. The organic layer was then dried over anhydrous Na₂SO₄, filtered and concentrated under vacuum. The residue was purified by prep HPLC.

### Step 6

Depending on the nature of R₃, different known reaction conditions were used to install R₃. When R₃═H Step 6 is skipped and the deprotection was performed using the conditions in Step 7.

### Step 7

To a solution of the above intermediate (1 equiv.) in 1,4-dioxane (0.1M) was added 4N HCl/1,4-dioxane (20 equiv.), and the mixture was stirred at RT until full deprotection. The mixture was concentrated under vacuum and the crude was purified by prep HPLC.

### General Procedure C

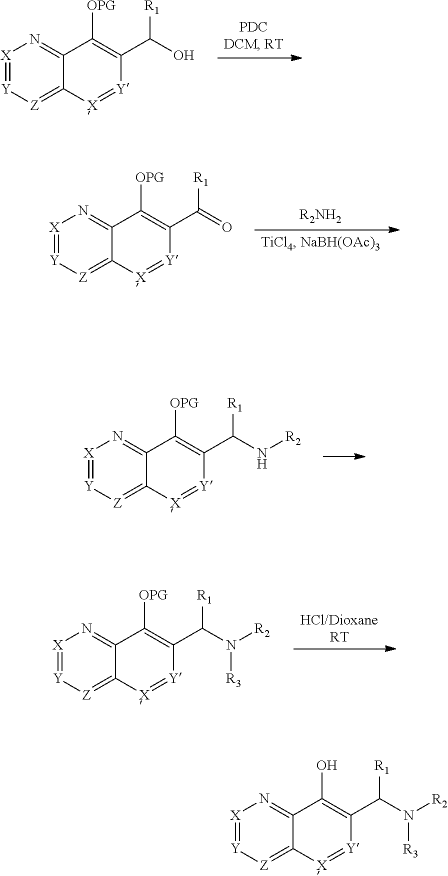

### Step 1

To a solution of the appropriate quinoline (1 eq, synthesized as in step 3 of general procedure B) in DCM (0.1M), PDC (1.2 equiv.) was added and reaction stirred at RT until full conversion. The mixture was diluted with DCM and washed with water and brine. The organic layer was then dried over anhydrous Na₂SO₄, filtered and concentrated under vacuum. The residue was purified by flash chromatography.

### Step 2

To a solution of the above intermediate (1 equiv.) in DCM (0.1M) TiCl₄ (1M in DCM, 1.2 equiv.) was added followed by the appropriate amine (1.5 eq). The resulting mixture was stirred at RT for few hours, then NaBH(OAc)₃ (2 equiv.) was added followed by methanol (⅓ volume of DCM). The reaction was then stirred at RT until full conversion. The reaction was quenched with saturated aq. NaHCO₃ solution and the aqueous layer was extracted with EtOAc (×3). The combined organic layers were washed with brine, dried over anhydrous Na₂SO₄, filtered and concentrated under vacuum. The residue was purified by flash chromatography or prep HPLC.

### Step 3

Depending on the nature of R₃, different known reaction conditions were used to install R₃. When R₃═H Step 3 is skipped and the deprotection was performed using the conditions in Step 4.

### Step 4

To a solution of the above intermediate (1 equiv.) in 1,4-dioxane (0.1M) was added 4N HCl/1,4-dioxane (20 equiv.), and the mixture was stirred at RT until full deprotection. The mixture was concentrated under vacuum and the crude was purified by prep HPLC.

### General Procedure D

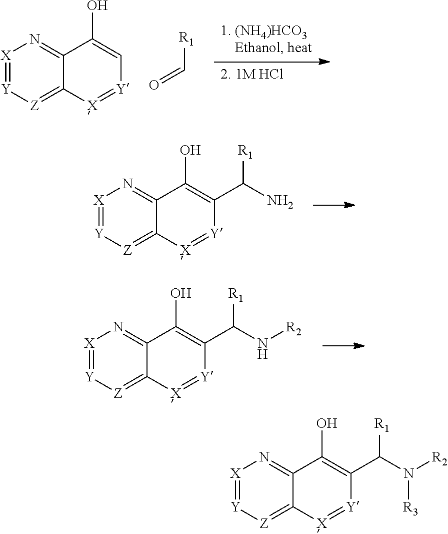

### Step 1-2

The appropriate quinoline (1 equiv.) and the appropriate aldehyde (2 equiv.) were dissolved in Ethanol (0.3M), then ammonium bicarbonate (3 equiv.) was added and reaction stirred overnight at 80° C. The reaction mixture was concentrated under vacuum and the residue was dissolved in 1M HCl (0.3M) and the resulting mixture was stirred at 80° C. for 1 hour and the crude product was purified by prep HPLC.

### Steps 2 & 3

Depending on the nature of R₂ and R₃, different known reaction conditions were used to install R₂ and R₃

### General Procedure E

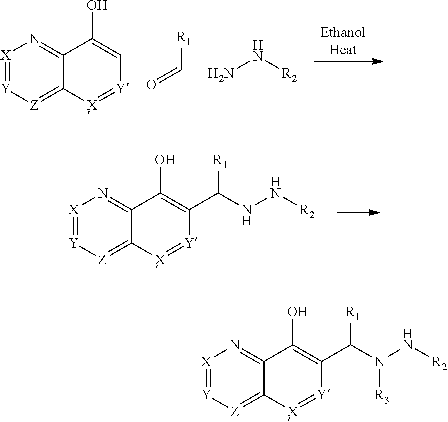

### Step 1

The appropriate aldehyde (1 equiv.) and hydrazine (1.1 equiv.) were dissolved in Ethanol (0.3M) and the reaction was stirred at RT for 10-15 min before the appropriate quinoline (1 equiv.) was added and the reaction was stirred overnight at 60-80° C. In some case microwave-assisted synthesis was used. The reaction mixture was concentrated under vacuum and the crude was purified by prep HPLC.

### Step 2

Depending on the nature of R₃, different known reaction conditions were used to install R₃

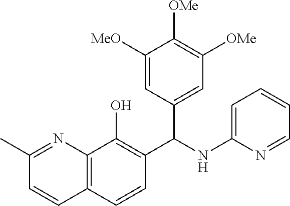 [SMILES: COc1cc(C(Nc2ccccn2)c2ccc3ccc(C)nc3c2O)cc(OC)c1OC]

This compound was synthesized using General Procedure A (LC/MS m/z 432.33 [M+H³⁰])

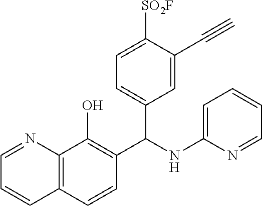 [SMILES: C#Cc1cc(C(Nc2ccccn2)c2ccc3cccnc3c2O)ccc1S(=O)(=O)F]

This compound was synthesized using General Procedure A (LC/MS m/z 434.37 [M+H⁺])

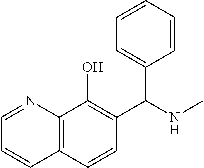 [SMILES: CNC(c1ccccc1)c1ccc2cccnc2c1O]

This compound was synthesized using General Procedure A (LC/MS m/z 265.09 [M+H⁺])

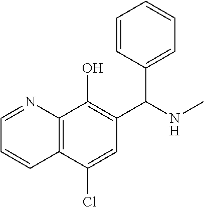 [SMILES: CNC(c1ccccc1)c1cc(Cl)c2cccnc2c1O]

This compound was synthesized using General Procedure A (LC/MS m/z 299.40 [M+H⁺])

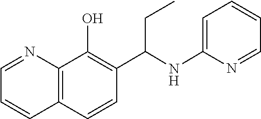 [SMILES: CCC(Nc1ccccn1)c1ccc2cccnc2c1O]

This compound was synthesized using General Procedure A (LC/MS m/z 280.34 [M+H⁺])

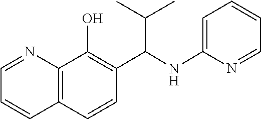 [SMILES: CC(C)C(Nc1ccccn1)c1ccc2cccnc2c1O]

This compound was synthesized using General Procedure A (LC/MS m/z 294.30 [M+H⁺])

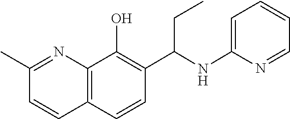 [SMILES: CCC(Nc1ccccn1)c1ccc2ccc(C)nc2c1O]

This compound was synthesized using General Procedure A (LC/MS m/z 294.33 [M+H⁺])

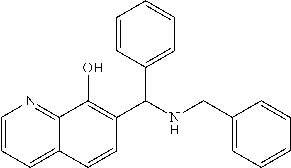 [SMILES: Oc1c(C(NCc2ccccc2)c2ccccc2)ccc2cccnc12]

This compound was synthesized using General Procedure B (LC/MS m/z 341.44 [M+H⁺])

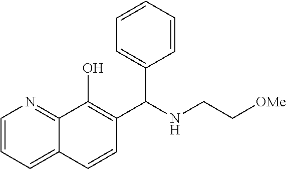 [SMILES: COCCNC(c1ccccc1)c1ccc2cccnc2c1O]

This compound was synthesized using General Procedure A (LC/MS m/z 309.43 [M+H⁺])

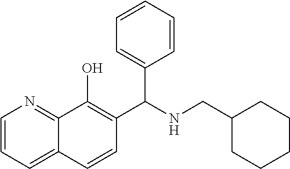 [SMILES: Oc1c(C(NCC2CCCCC2)c2ccccc2)ccc2cccnc12]

This compound was synthesized using General Procedure A (LC/MS m/z 347.47 [M+H⁺])

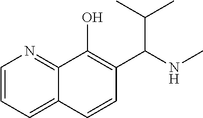 [SMILES: CNC(c1ccc2cccnc2c1O)C(C)C]

This compound was synthesized using General Procedure A (LC/MS m/z 231.12 [M+H⁺])

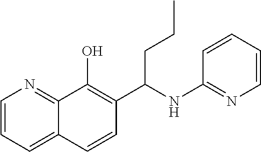 [SMILES: CCCC(Nc1ccccn1)c1ccc2cccnc2c1O]

This compound was synthesized using General Procedure A (LC/MS m/z 294.37 [M+H⁺])

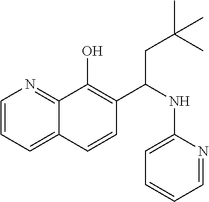 [SMILES: CC(C)(C)CC(Nc1ccccn1)c1ccc2cccnc2c1O]

This compound was synthesized using General Procedure A (LC/MS m/z 322.42 [M+H⁺])

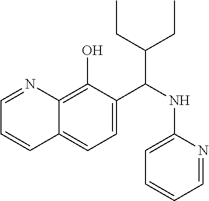 [SMILES: CCC(CC)C(Nc1ccccn1)c1ccc2cccnc2c1O]

This compound was synthesized using General Procedure A (LC/MS m/z 322.49 [M+H⁺])

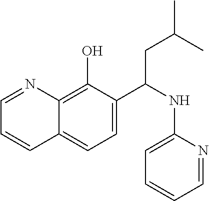 [SMILES: CC(C)CC(Nc1ccccn1)c1ccc2cccnc2c1O]

This compound was synthesized using General Procedure A (LC/MS m/z 308.39 [M+H⁺])

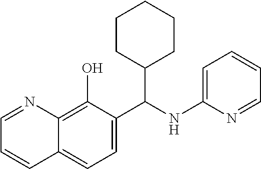 [SMILES: Oc1c(C(Nc2ccccn2)C2CCCCC2)ccc2cccnc12]

This compound was synthesized using General Procedure A (LC/MS m/z 334.41 [M+H⁺])

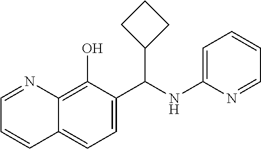 [SMILES: Oc1c(C(Nc2ccccn2)C2CCC2)ccc2cccnc12]

This compound was synthesized using General Procedure A (LC/MS m/z 306.39 [M+H⁺])

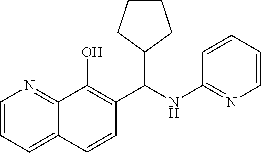 [SMILES: Oc1c(C(Nc2ccccn2)C2CCCC2)ccc2cccnc12]

This compound was synthesized using General Procedure A (LC/MS m/z 320.46 [M+H⁺])

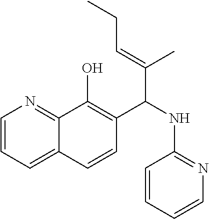 [SMILES: CC/C=C(\C)C(Nc1ccccn1)c1ccc2cccnc2c1O]

This compound was synthesized using General Procedure A (LC/MS m/z 320.46 [M+H⁺])

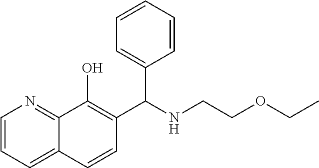 [SMILES: CCOCCNC(c1ccccc1)c1ccc2cccnc2c1O]

This compound was synthesized using General Procedure A (LC/MS m/z 323.45 [M+H⁺])

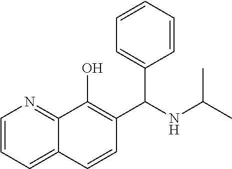 [SMILES: CC(C)NC(c1ccccc1)c1ccc2cccnc2c1O]

This compound was synthesized using General Procedure B (LC/MS m/z 293.40 [M+H⁺])

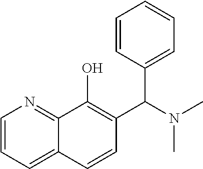 [SMILES: CN(C)C(c1ccccc1)c1ccc2cccnc2c1O]

This compound was synthesized using General Procedure B (LC/MS m/z 279.38 [M+H³⁰])

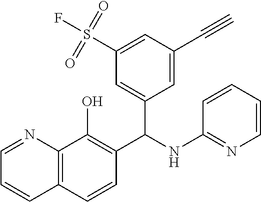 [SMILES: C#Cc1cc(C(Nc2ccccn2)c2ccc3cccnc3c2O)cc(S(=O)(=O)F)c1]

This compound was synthesized using General Procedure A (LC/MS m/z 434.33 [M+H³⁰])

### General Procedure 1

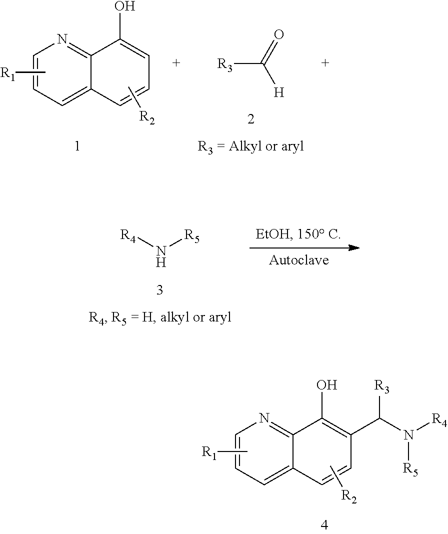

A solution of the appropriate 8-hydroxyquinoline 1 (1 eq.), alkyl or aryl aldehyde 2 (1.2 eq.) and alkyl or aryl amine 3 (1.2 eq.) in EtOH (0.1M) was transferred to an autoclave reactor, which was then sealed and heated to 150° C. for 12 hours. The reaction mixture was concentrated under vacuum and the residue was purified by reverse-phase flash column chromatography, prep-TLC or prep-HPLC to give the desired compound 4.

### Example: Synthesis of Compound 5

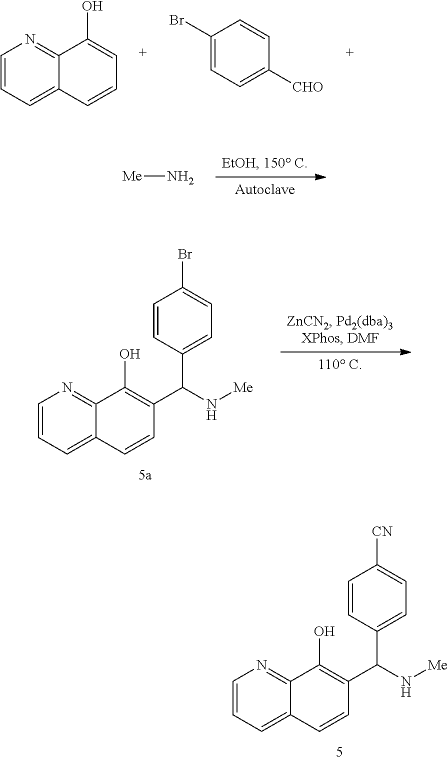

### Step 1

A solution of 8-hydroxyquinoline (290 mg, 2.0 mmol), methyl amine (2.0M in EtOH, 1.2 mL) and 4-bromobenzaldehyde (442 mg, 2.4 mmol) in EtOH (20 mL) was transferred to an autoclave reactor, which was then sealed and heated to 150° C. for 12 hours. The reaction mixture was concentrated under vacuum and the residue was purified by reverse-phase flash column chromatography, followed by prep-HPLC to give 5a (25 mg, yield: 3.6%) as a white solid.

### Step 2

ZnCN₂ (16 mg, 0.14 mmol), Pd₂(dba)₃ (8 mg, 0.01 mmol) and XPhos (6 mg, 0.01 mmol) were added under N₂ atmosphere to a solution of 5a (25 mg, 0.07 mmol) in DMF (2 mL), and the mixture was heated to 110° C. for 8 hours. The reaction was cooled to room temperature, concentrated under vacuum and the residue was purified by prep-HPLC to give 5 (4 mg, yield: 20%) as a white solid.

### General Procedure 2

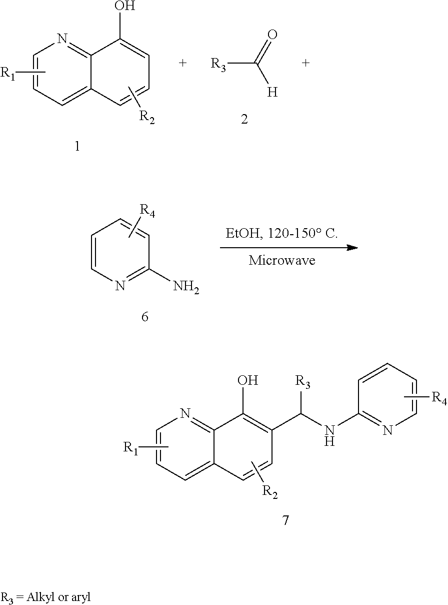

A solution of the appropriate 8-hydroxyquinoline 1 (1 eq.), alkyl or aryl aldehyde 2 (1.2 eq.) and 2-aminopyridine 6 (1.2 eq.) in EtOH (0.1M) was transferred to a microwave reaction vial, which was then sealed and stirred under microwave condition at 120-150° C. (150° C. for alkyl aldehyde) for 2 to 6 hours. The reaction mixture was then cooled to room temperature and concentrated under vacuum. The residue was purified by reverse-phase flash column chromatography, prep-TLC or prep-HPLC to give compound 7.

### Example: Synthesis of Compound 8

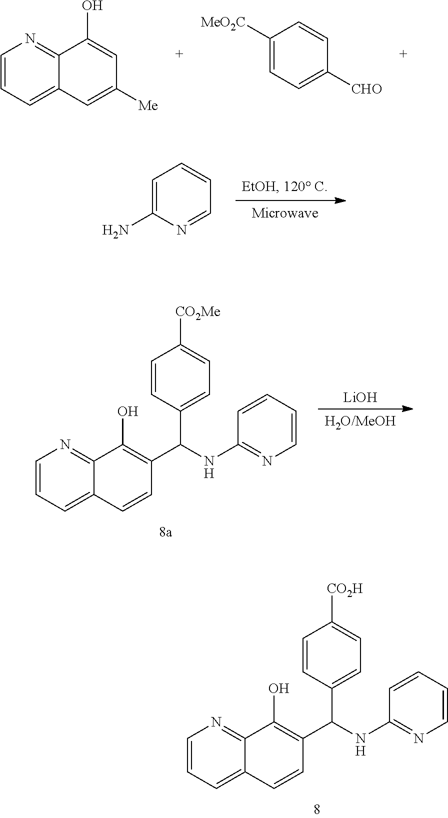

### Step 1

A solution of 6-methyl-8-hydroxyquinoline (160 mg, 1 mmol), methyl 4-formylbenzoate (197 mg, 1.2 mmol) and 2-aminopyridine (110 mg, 1.2 mmol) in EtOH (10 mL) was transferred to a microwave reaction vial, which was then sealed and stirred under microwave condition at 120° C. for 2 hours. The reaction mixture was then cooled to room temperature and concentrated under vacuum. The residue was purified by reverse-phase flash column chromatography, followed by prep-TLC to give 8a (40 mg, yield: 10%) as a light yellow solid.

### Step 2

8a (40 mg, 0.1 mmol) was added to a solution of lithium hydroxide monohydrate (10 mg, 0.24 mmol) in methanol (2 mL) and H₂O (2 mL), and the resulting mixture was stirred overnight at room temperature. The mixture was then acidified with aq. HCl (1N) solution to pH=4 and concentrated under vacuum. The residue was purified by prep-HPLC to give 8 (12.6 mg, yield: 33%) as a yellow solid.

### General Procedure 3

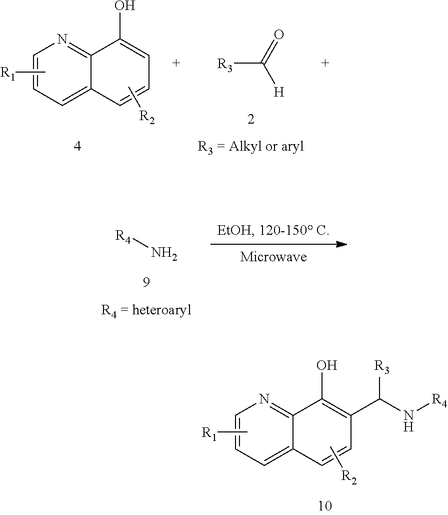

A solution of the appropriate 8-hydroxyquinoline 1 (1 eq.), alkyl or aryl aldehyde 2 (1.2 eq.) and heteroaryl amine 9 (1.2 eq.) in EtOH (0.1M) was transferred to a microwave reaction vial, which was then sealed and stirred under microwave condition at 120° C. (150° C. for alkyl aldehydes) for 2 to 6 hours. The reaction mixture was then cooled to room temperature and concentrated under vacuum. The residue was purified by reverse-phase flash column chromatography, prep-TLC or prep-HPLC to give compound 10.

### Example: Synthesis of Compound 11

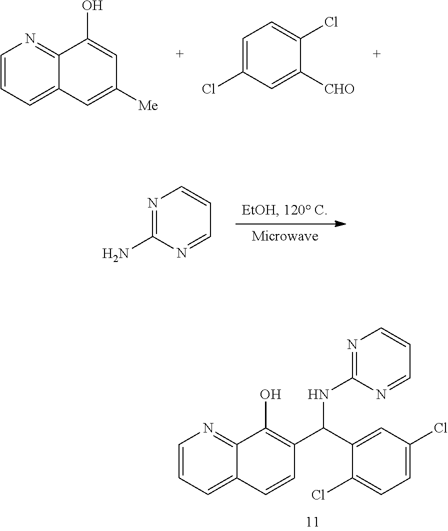 [SMILES: Cc1cc(O)c2ncccc2c1 ; O=Cc1cc(Cl)ccc1Cl ; Nc1ncccn1 ; "EtOH, 120° C." ; "Microwave" ; Oc1c(C(Nc2ncccn2)c2cc(Cl)ccc2Cl)ccc2cccnc12]

A solution of 6-methyl-8-hydroxyquinoline (160 mg, 1 mmol), 2,5-dichlorobenzaldehyde (210 mg, 1.2 mmol) and 2-aminopyrimidine (110 mg, 1.2 mmol) in EtOH (10 mL) was transferred to a microwave reaction vial, which was then sealed and heated to 120° C. under microwave conditions for 4 hours. The reaction mixture was cooled to room temperature and concentrated under vacuum. The residue was purified by reverse-phase flash column chromatography, followed by prep-TLC to give the desired compound 11 (5.9 mg, yield: 1.5%) as a yellow solid.

### General Procedure 4

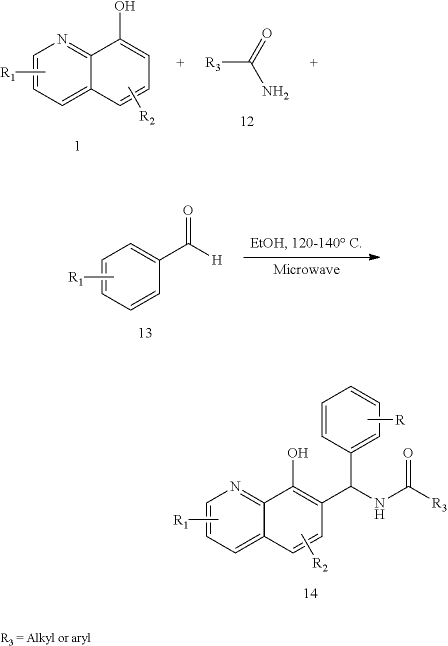

A solution of the appropriate 8-hydroxyquinoline 1 (1 eq.), alkyl or aryl amide 12 (1.2 eq.) and aryl aldehyde 13 (1.2 eq.) in EtOH (0.1M) was transferred to a microwave reaction vial, which was then sealed and stirred under microwave condition at 120-140° C. for 2 to 12 hours (alkyl amides required longer reaction time). The reaction mixture was then cooled to room temperature and concentrated under vacuum. The reside was purified by reverse-phase flash column chromatography, prep-TLC or prep-HPLC to give compound 14.

### Example: Synthesis of Compound 15

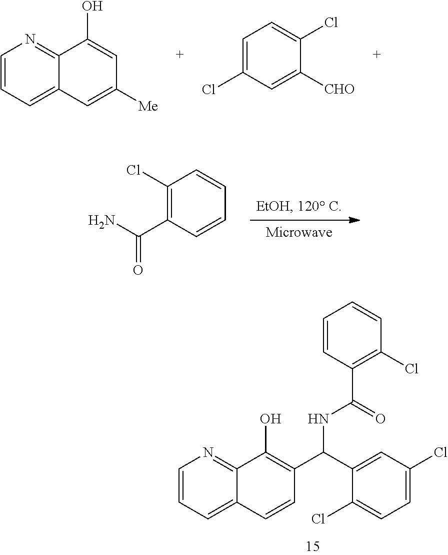 [SMILES: Cc1cc(O)c2ncccc2c1 ; O=Cc1cc(Cl)ccc1Cl ; NC(=O)c1ccccc1Cl ; "EtOH, 120° C." ; "Microwave" ; O=C(NC(c1cc(Cl)ccc1Cl)c1ccc2cccnc2c1O)c1ccccc1Cl]

A solution of 6-methyl-8-hydroxyquinoline (160 mg, 1 mmol), 2,5-dichlorobenzaldehyde (210 mg, 1.2 mmol) and 2-chlorobenzamide (186 mg, 1.2 mmol) in EtOH (10 mL) was transferred to a microwave reaction vial, which was then sealed and stirred under microwave condition at 120° C. for 2 hours. The reaction mixture was then cooled to room temperature and concentrated under vacuum. The reside was purified by reverse-phase flash column chromatography followed by prep-HPLC to give 15 (4.9 mg, yield: 1.5%) as a white solid.

### General Procedure 5

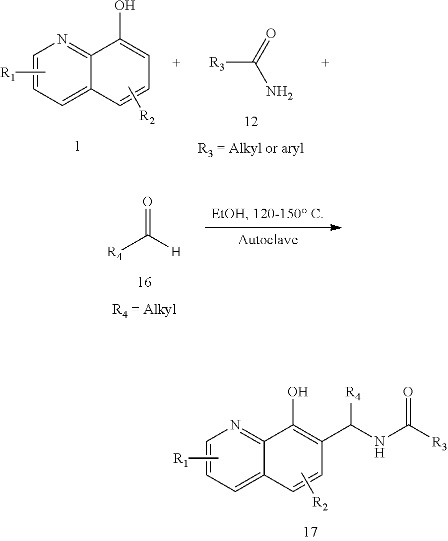

A solution of the appropriate 8-hydroxyquinoline 1 (1 eq.), alkyl or aryl amide 12 (1.2 eq.), and alkyl aldehyde 16 (1.2 eq.) in EtOH (0.1M) was transferred to an autoclave reactor, which was then sealed and heated to 120-150° C. for 12 to 24 hours. The reaction mixture was concentrated under vacuum and the residue was purified by reverse-phase flash column chromatography, prep-TLC or prep-HPLC to give compound 17.

### Example: Synthesis of Compound 18

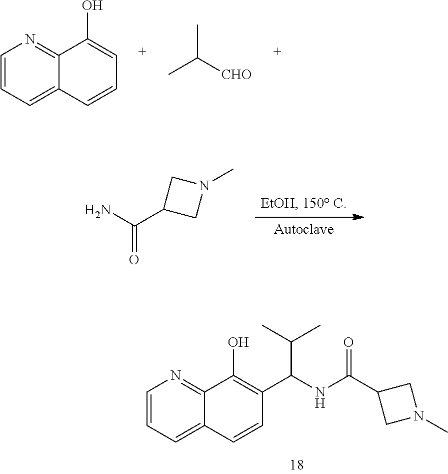

A solution of 8-hydroxyquinoline (145 mg, 1 mmol), isobutyraldehyde (85 mg, 1.2 mmol) and 1-methylazetidine-3-carboxamide (140 mg, 1.2 mmol) in EtOH (10 mL) was transferred to an autoclave reactor, which was then sealed and heated to 150° C. for 12 hours. The reaction mixture was concentrated under vacuum and the residue was purified by reverse-phase flash column chromatography, followed by prep-HPLC to give 18 (11 mg, yield: 3.5%) as a white solid.

### General Procedure 6

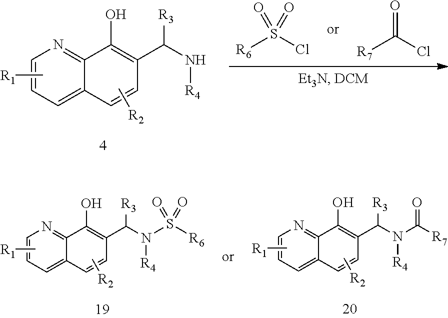

Compound 4 was prepared according to general procedure 1

The appropriate compound 4 (1 eq.) was dissolved in DCM (0.05M), then Et₃N (2 eq.) was added followed by the appropriate sulfonyl chloride or acyl chloride (1.2 eq.) at 0° C. The reaction was slowly warmed to room temperature and stirred for 15-30 minutes. The mixture was concentrated under vacuum and the residue was purified by prep-HPLC to give compound 19 or 20.

### Example: Synthesis of Compound 21

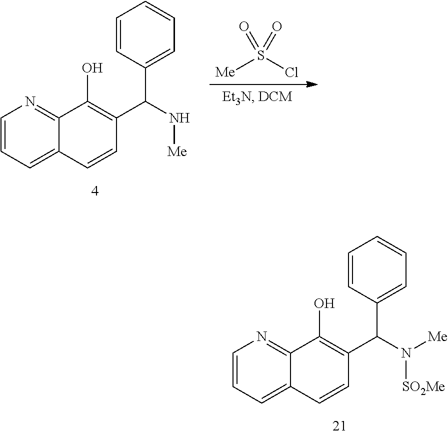

21a (6.0 mg, 0.023 mmol) was dissolved in DCM (0.5 mL), then Et₃N (6.33 uL, 0.046 mmol) was added followed by methanesulfonyl chloride (5 uL, 0.028 mmol) at 0° C. The reaction was slowly warmed to room temperature and stirred for 30 minutes. The mixture was concentrated under vacuum and the residue was purified by prep-HPLC to give 21 as a white solid.

| | General | |
|---|---|---|
| Compound | procedure | Experimental data |
| 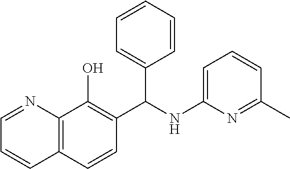 [SMILES: Cc1cccc(NC(c2ccccc2)c2ccc3cccnc3c2O)n1] | 2 | 7-(((6-methylpyridin-2- yl)amino)(phenyl)methyl)quinolin-8-ol LC-MS: m/z 342 (M + H)⁺. 1H NMR (400 MHz, DMSO-d6) δ 9.91 (brs, 1H), 8.78 (dd, J = 4.2, 1.7 Hz, 1H), 8.25-8.17 (m, 1H), 7.58 (d, J = 8.6 Hz, 1H), 7.46 (dd, J = 8.3, 4.2 Hz, 1H), 7.35-7.27 (m, 3H), 7.24-7.15 (m, 4H), 7.13- 7.07 (m, 1H), 6.75 (d, J = 8.8 Hz, 1H), 6.36 (d, J = 8.3 Hz, 1H), 6.27 (d, J = 7.1 Hz, 1H), 2.14 (s, 3H). |
| 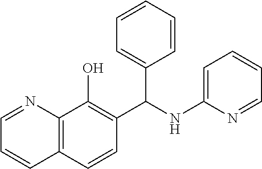 [SMILES: Oc1c(C(Nc2ccccn2)c2ccccc2)ccc2cccnc12] | 2 | 7-(phenyl(pyridin-2-ylamino)methyl)quinolin-8-ol LC-MS: m/z 328 (M + H)⁺. 1H NMR (400 MHz, DMSO-d6) δ 9.98 (brs, 1H), 8.85 (dd, J = 4.1, 1.8 Hz, 1H), 8.29 (dd, J = 8.4, 1.8 Hz, 1H), 7.91 (d, J = 5.0 Hz, 1H), 7.62 (d, J = 8.4 Hz, 1H), 7.53 (dd, J = 8.3, 4.3 Hz, 1H), 7.45-7.33 (m, 5H), 7.31-7.27 (m, 2H), 7.21-7.17 (m, 1H), 6.86 (d, J = 8.4 Hz, 1H), 6.68 (d, J = 8.5 Hz, 1H), 6.48-6.45 (m, 1H). |
| 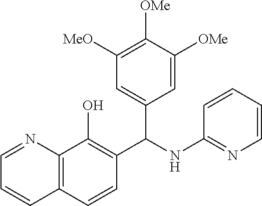 [SMILES: COc1cc(C(Nc2ccccn2)c2ccc3cccnc3c2O)cc(OC)c1OC] | 2 | 7-((pyridin-2-ylamino)(3,4,5- trimethoxyphenyl)methyl)quinolin-8-ol LC-MS: m/z 418 (M + H)⁺. 1H NMR (400 MHz, DMSO-d₆) δ 9.94 (brs, 1H), 8.85 (dd, J = 4.2, 1.8 Hz, 1H), 8.36-8.23 (m, 1H), 7.92 (d, J = 4.8 Hz, 1H), 7.64 (d, J = 8.5 Hz, 1H), 7.53 (dd, J = 8.3, 4.2 Hz, 1H), 7.42-7.28 (m, 3H), 6.77-6.73 (m, 3H), 6.67 (d, J = 8.5 Hz, 1H), 6.49- 6.46 (m, 1H), 3.70 (d, J = 1.7 Hz, 6H), 3.60 (d, J = 1.7 Hz, 3H).-No references, but can buy it |
| 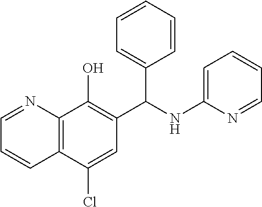 [SMILES: Oc1c(C(Nc2ccccn2)c2ccccc2)cc(Cl)c2cccnc12] | 2 | 5-chloro-7-(phenyl(pyridin-2- ylamino)methyl)quinolin-8-ol LC-MS: m/z 362 (M + H)⁺. 1H NMR (400 MHz, DMSO-d6) δ 10.29 (brs, 1H), 8.95 (dd, J = 4.2, 1.5 Hz, 1H), 8.46 (dd, J = 8.6, 1.6 Hz, 1H), 7.92 (dd, J = 5.1, 1.8 Hz, 1H), 7.78 (s, 1H), 7.71 (dd, J = 8.6, 4.2 Hz, 1H), 7.47 (d, J = 8.7 Hz, 1H), 7.44-7.27 (m, 5H), 7.23-7.20 (m, 1H), 6.87 (d, J = 8.5 Hz, 1H), 6.69 (d, J = 8.4 Hz, 1H), 6.49 (dd, J = 7.0, 5.1 Hz, 1H). |
| 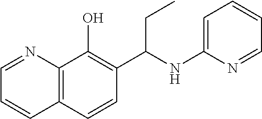 [SMILES: CCC(Nc1ccccn1)c1ccc2cccnc2c1O] | A | 7-(1-(pyridin-2-ylamino)propyl)quinolin-8-ol LC-MS: m/z 280 (M + H)⁺. 1H NMR (400 MHz, DMSO-d6) δ 9.77 (brs, 1H), 8.77 (dd, J = 4.2, 1.6 Hz, 1H), 8.18 (dd, J = 8.3, 1.6 Hz, 1H), 7.79 (dd, J = 5.1, 1.8 Hz, 1H), 7.48 (d, J = 8.5 Hz, 1H), 7.44 (dd, J = 8.3, 4.2 Hz, 1H), 7.30- 7.20 (m, 2H), 6.92 (d, J = 8.3 Hz, 1H), 6.42 (d, J = 8.4 Hz, 1H), 6.32 (dd, J = 7.0, 5.1 Hz, 1H), 5.23- |
| | | 5.18 (m, 1H), 1.80-1.72 (m, 2H), 0.87 (t, J = 7.3 |
| | | Hz, 3H). |
| 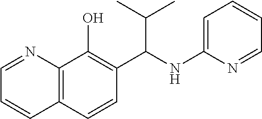 [SMILES: CC(C)C(Nc1ccccn1)c1ccc2cccnc2c1O] | A | 7-(2-methyl-1-(pyridin-2-ylamino)propyl)quinolin- 8-ol LC-MS: m/z 294 (M + H)⁺. 1H NMR (400 MHz, DMSO-d6) δ 9.75 (brs, 1H), 8.76 (dd, J = 4.2, 1.6 Hz, 1H), 8.19 (dd, J = 8.3, 1.6 Hz, 1H), 7.79 (dd, J = 5.2, 1.8 Hz, 1H), 7.49 (d, J = 8.5 Hz, 1H), 7.44 (dd, J = 8.3, 4.2 Hz, 1H), 7.28 (d, J = 8.5 Hz, 1H), 7.22 (ddd, J = 8.8, 7.0, 2.0 Hz, |
| | | 1H), 6.85 (d, J = 9.0 Hz, 1H), 6.46 (d, J = 8.5 Hz, |
| | | 1H), 6.31 (dd, J = 7.0, 5.0 Hz, 1H), 5.13 (t, J = 8.4 |
| | | Hz, 1H), 2.11 (dq, J = 13.7, 6.8 Hz, 1H), 0.95 (d, J = |
| | | 6.7 Hz, 3H), 0.76 (d, J = 6.7 Hz, 3H). |
| 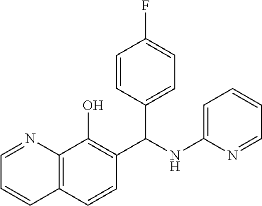 [SMILES: Oc1c(C(Nc2ccccn2)c2ccc(F)cc2)ccc2cccnc12] | 2 | 7-((4-fluorophenyl)(pyridin-2- ylamino)methyl)quinolin-8-ol LC-MS: m/z 346 (M + H)⁺. 1H NMR (400 MHz, DMSO-d6) δ 9.98 (brs, 1H), 8.85 (dd, J = 4.3, 1.6 Hz, 1H), 8.29 (dd, J = 8.4, 1.6 Hz, 1H), 7.91 (dd, J = 5.2, 1.8 Hz, 1H), 7.61 (d, J = 8.5 Hz, 1H), 7.53 (dd, J = 8.3, 4.2 Hz, 1H), 7.41- 7.35 (m, 5H), 7.15-7.08 (m, 2H), 6.85 (d, J = 8.4 Hz, 1H), 6.67 (d, J = 8.4 Hz, 1H), 6.47 (dd, J = 7.0, 5.1 Hz, 1H). |
| 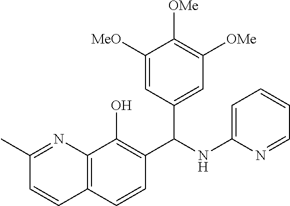 [SMILES: COc1cc(C(Nc2ccccn2)c2ccc3ccc(C)nc3c2O)cc(OC)c1OC] | A | 2-methyl-7-((pyridin-2-ylamino)(3,4,5- trimethoxyphenyl)methyl)quinolin-8-ol LC-MS: m/z 432 (M + H)⁺. 1H NMR (400 MHz, DMSO-d6) δ 9.46 (brs, 1H), 8.15 (d, J = 8.4 Hz, 1H), 7.92 (dd, J = 5.3, 1.9 Hz, 1H), 7.56 (d, J = 8.5 Hz, 1H), 7.42-7.30 (m, 4H), 6.77-6.73 (m, 3H), 6.66 (d, J = 8.5 Hz, 1H), 6.47 (dd, J = 6.8, 5.3 Hz, 1H), 3.69 (s, 6H), 3.59 (s, 3H), 2.69 (s, 3H). |
| 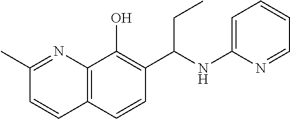 [SMILES: CCC(Nc1ccccn1)c1ccc2ccc(C)nc2c1O] | A | 2-methyl-7-(1-(pyridin-2-ylamino)propyl)quinolin- 8-ol LC-MS: m/z 294 (M + H)⁺. 1H NMR (400 MHz, DMSO-d6) δ 9.39 (brs, 1H), 8.13 (d, J = 8.4 Hz, 1H), 7.87 (dd, J = 5.1, 1.8 Hz, 1H), 7.47 (d, J = 8.5 Hz, 1H), 7.38 (d, J = 8.4 Hz, 1H), 7.34-7.27 (m, 2H), 6.98 (d, J = 8.2 Hz, 1H), 6.48 (d, J = 8.5 Hz, 1H), 6.39 (dd, J = 7.0, 5.1 Hz, |
| | | 1H), 5.24 (q, J = 7.6 Hz, 1H), 2.69 (s, 3H), 1.92- |
| | | 1.75 (m, 2H), 0.93 (t, J = 7.3 Hz, 3H). |
| 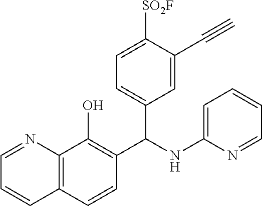 [SMILES: C#Cc1cc(C(Nc2ccccn2)c2ccc3cccnc3c2O)ccc1S(=O)(=O)F] | A | 2-ethynyl-4-((8-hydroxyquinolin-7-yl)(pyridin-2- ylamino)methyl)benzenesulfonyl fluoride LC-MS: m/z 434 (M + H)⁺. 1H NMR (400 MHz, DMSO-d6) δ 8.88 (dd, J = 4.2, 1.6 Hz, 1H), 8.37-8.28 (m, 1H), 8.11 (d, J = 8.4 Hz, 1H), 7.98-7.90 (m, 1H), 7.83 (d, J = 1.8 Hz, 1H), 7.70 (d, J = 8.4 Hz, 1H), 7.58-7.56 (m, 3H), 7.45-7.38 (m, 2H), 6.95 (d, J = 7.9 Hz, 1H), 6.74 (d, J = 8.3 Hz, 1H), 6.58-6.47 (m, 1H), 4.92 (s, 1H). |
| 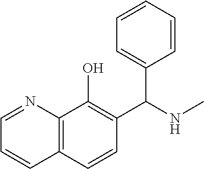 [SMILES: CNC(c1ccccc1)c1ccc2cccnc2c1O] | A | 7-((methylamino)(phenyl)methyl)quinolin-8-ol LC-MS: m/z 265 (M + H)⁺. 1H NMR (400 MHz, DMSO-d6) δ 8.82 (d, J = 4.1 Hz, 1H), 8.25 (d, J = 8.5 Hz, 1H), 7.56 (d, J = 8.6 Hz, 1H), 7.53-7.42 (m, 3H), 7.38-7.25 (m, 3H), 7.21-7.18 (m, 1H), 5.25 (s, 1H), 2.31 (s, 3H). |
| 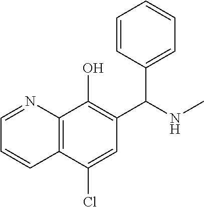 [SMILES: CNC(c1ccccc1)c1cc(Cl)c2cccnc2c1O] | A | 5-chloro-7- ((methylamino)(phenyl)methyl)quinolin-8-ol LC-MS: m/z 299 (M + H)⁺. 1H NMR (400 MHz, DMSO-d6) δ 8.85 (dd, J = 4.2, 1.6 Hz, 1H), 8.39-8.32 (m, 2H), 7.66 (s, 1H), 7.60 (dd, J = 8.5, 4.2 Hz, 1H), 7.41-7.36 (m, 2H), 7.23 (dd, J = 7.5, 7.5 Hz, 2H), 7.15-7.11 (m, 1H), 5.15 (s, 1H), 2.22 (s, 3H). |
| 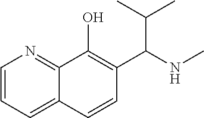 [SMILES: CNC(c1ccc2cccnc2c1O)C(C)C] | A | 7-(2-methyl-1-(methylamino)propyl)quinolin-8-ol LC-MS: m/z 231 (M + H)⁺. 1H NMR (400 MHz, DMSO-d₆) δ 8.83 (dd, J = 4.2, 1.6 Hz, 1H), 8.32-8.24 (m, 2H), 7.52 (dd, J = 8.3, 4.1 Hz, 1H), 7.46-7.34 (m, 2H), 3.93 (d, J = 6.7 Hz, 1H), 2.22 (s, 3H), 2.12-2.05 (m, 1H), 0.99 (d, J = 6.7 Hz, 3H), 0.80 (d, J = 6.8 Hz, 3H). |
| 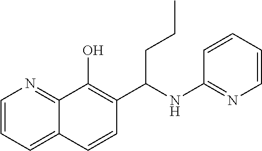 [SMILES: CCCC(Nc1ccccn1)c1ccc2cccnc2c1O] | A | 7-(1-(pyridin-2-ylamino)butyl)quinolin-8-ol LC-MS: m/z 294 (M + H)⁺. 1H NMR (400 MHz, DMSO-d6) δ (ppm): 9.90 (br s, 1H), 8.85-8.83 (m, 1H), 8.27-8.24 (m, 1H), 7.57 (d, J = 8.0 Hz, 1H), 7.36 (d, J = 8.0 Hz, 1H), 6.99 (d, J = 8.0 Hz, 1H), 6.50 (d, J = 8.0 Hz, 1H), 6.41- 6.38 (m, 2H), 5.41-5.35 (m, 1H), 1.81-1.77 (m, 2H), 1.53-1.26 (m, 2H), 0.92 (t, J = 8.0 Hz, 3H). |
| 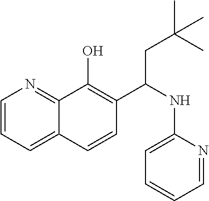 [SMILES: CC(C)(C)CC(Nc1ccccn1)c1ccc2cccnc2c1O] | A | 7-(3,3-dimethyl-1-(pyridin-2- ylamino)butyl)quinolin-8-ol LC-MS: m/z 322 (M + H)⁺. 1H NMR (400 MHz, DMSO-d6) δ (ppm): 10.00 (br s, 1H), 8.84-8.82 (m, 1H), 8.26 (d, J = 8.0 Hz, 1H), 7.63 (d, J = 8.0 Hz, 1H), 7.51 (t, J = 4.0 Hz, 1H), 7.36 (d, J = 8.0 Hz, 1H), 7.35-7.25 (m, 2H), 6.94 (d, J = 8.0 Hz, 1H), 6.51 (d, J = 8.0 Hz, 1H), 6.40-6.37 (m, 1H), 5.57-5.53 (m, 1H), 1.86-1.83 (m, 1H), 1.67-1.62 (m, 1H), 0.98 (s, 9H). |
| 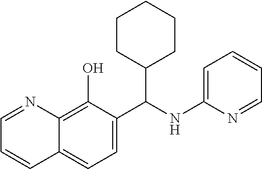 [SMILES: Oc1c(C(Nc2ccccn2)C2CCCCC2)ccc2cccnc12] | A | 7-(cyclohexyl(pyridin-2-ylamino)methyl)quinolin- 8-ol LC-MS: m/z 334 (M + H)⁺. 1H NMR (400 MHz, DMSO-d6) δ (ppm): 9.81 (br s, 1H), 8.84-8.83 (m, 1H), 8.27-8.24 (m, 1H), 7.87 (d, J = 4.0 Hz, 1H), 7.57-7.49 (m, 2H), 7.36 (d, J = 8.0 Hz, 1H), 7.35-7.25 (m, 2H), 7.30 (d, J = 8.0 Hz, 1H), 6.53 (d, J = 8.0 Hz, 1H), 6.39-6.36 (m, 1H), 5.28 (t, J = 8.0 Hz, 1H), 2.19-1.60 (m, 5H), 1.24-1.10 (m, 6H). |
| 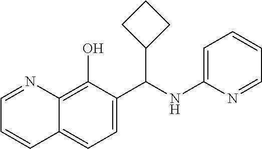 [SMILES: Oc1c(C(Nc2ccccn2)C2CCC2)ccc2cccnc12] | A | 7-(cyclobutyl(pyridin-2-ylamino)methyl)quinolin- 8-ol LC-MS: m/z 306 (M + H)⁺. 1H NMR (400 MHz, DMSO-d6) δ (ppm): 9.93 (br s, 1H), 8.85-8.83 (m, 1H), 8.27 (t, J = 4.0 Hz, 1H), 7.80 (d, J = 4.0 Hz, 1H), 7.58 (d, J = 8.0 Hz, 1H), 7.53 (t, J = 4.0 Hz, 1H), 7.49-7.35 (m, 2H), 7.10 (s, 1H), 6.53(d, J = 8.0 Hz, 1H), 6.43 (t, J = 4.0 Hz, 1H), 5.47 (t, J = 8.0 Hz, 1H), 1.76-1.61 (m, 3H), 0.95-0.92 (m, 6H). |
| 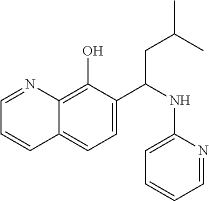 | A | 7-(2-methyl-1-(pyridin-2-ylamino)propyl)quinolin- 8-ol LC-MS: m/z 294 (M + H)⁺. 1H NMR (400 MHz, DMSO-d6) δ (ppm): 9.93 (br s, 1H), 8.85-8.83 (m, 1H), 8.27 (t, J = 4.0 Hz, 1H), 7.80 (d, J = 4.0 Hz, 1H), 7.58 (d, J = 8.0 Hz, 1H), 7.53 (t, J = 4.0 Hz, 1H), 7.49-7.35 (m, 2H), 7.10 (s, 1H), 6.53(d, J = 8.0 Hz, 1H), 6.43 (t, J = 4.0 Hz, 1H), 5.47 (t, J = 4.0 Hz, 1H), 1.76-1.61 (m, 3H), 0.95-0.92 (m, 6H). |
| 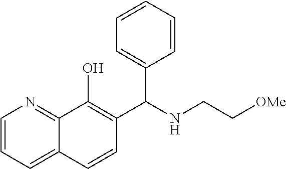 [SMILES: COCCNC(c1ccccc1)c1ccc2cccnc2c1O] | A | 7-(((2- methoxyethyl)amino)(phenyl)methyl)quinolin-8-ol LC-MS: m/z 309 (M + H)⁺. 1H NMR (400 MHz, DMSO-d6) δ (ppm): 8.83 (d, J = 2.8 Hz, 1H), 8.27 (t, J = 1.2 Hz, 1H), 7.60 (d, J = 8.0 Hz, 1H), 7.53-7.46 (m, 3H), 7.38-7.19 (m, 4H), 5.40 (s, 1H), 3.48-3.45 (m, 2H), 3.24 (s, 3H), 2.68-2.65 (m, 2H). |
| 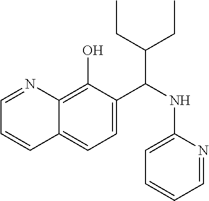 [SMILES: CCC(CC)C(Nc1ccccn1)c1ccc2cccnc2c1O] | A | 7-(2-ethyl-1-(pyridin-2-ylamino)butyl)quinolin-8- ol LC-MS: m/z 322 (M + H)⁺. 1H NMR (400 MHz, DMSO-d6) δ (ppm): 10.08 (br s, 1H), 8.84 (t, J = 1.6 Hz, 1H), 8.26 (t, J = 1.6 Hz, 1H), 7.86(d, J = 4.0 Hz, 1H), 7.59-7.49 (m, 2H), 7.36-7.28 (m, 2H), 6.85 (d, J = 9.2 Hz, 1H), 6.58 (d, J = 8.4 Hz, 1H), 6.39-6.36 (m, 1H), 5.49 (t, J = 8.0 Hz, 1H), 1.89-1.85 (m, 1H), 1.56-1.49 (m, 2H), 1.30-1.21 (m, 2H), 0.84-0.79 (m, 6H). |
| 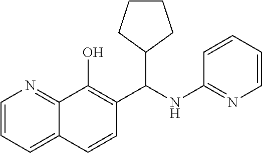 [SMILES: Oc1c(C(Nc2ccccn2)C2CCCC2)ccc2cccnc12] | A | 7-(cyclopentyl(pyridin-2-ylamino)methyl)quinolin- 8-ol LC-MS: m/z 320 (M + H)⁺. 1H NMR (400 MHz, DMSO-d6) δ (ppm): 9.18 (s, 1H), 8.90-8.89 (m, 1H), 8.36 (t, J = 8.0 Hz, 1H), 8.00 (d, J = 4.0 Hz, 1H), 7.90-7.80 (m, 1H), 7.60 (d, J = 8.0 Hz, 2H), 7.48 (d, J = 8.0 Hz, 1H), 6.99 (d, J = 8.0 Hz 1H), 6.83 (t, J = 8.0 Hz, 1H), 5.06 (s, 1H), 2.67-2.61 (m, 1H), 1.95-1.91 (m, 1H), 1.68- 1.23 (m, 7H). |
| 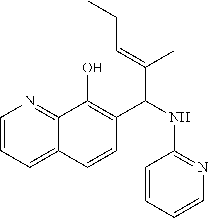 [SMILES: CC/C=C(\C)C(Nc1ccccn1)c1ccc2cccnc2c1O] | A | (Z)-7-(2-methyl-1-(pyridin-2-ylamino)pent-2-en-1- yl)quinolin-8-ol LC-MS: m/z 320 (M + H)⁺. 1H NMR (400 MHz, CD3OD) δ (ppm): 8.86-8.85 (m, 1H), 8.30 (t, J = 4.0 Hz, 1H), 7.93-7.85 (m, 2H), 7.60-7.42 (m, 3H), 7.13 (d, J = 8.0 Hz, 1H), 6.94 (t, J = 8.0 Hz, 1H), 5.82 (s, 1H), 5.61 (s, 1H), 2.30-2.10 (m, 2H), 1.68 (s, 3H), 1.0 (t, J = 8.0 Hz, 3H). |
| 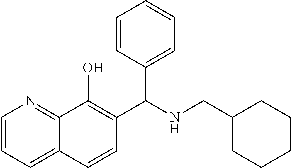 [SMILES: Oc1c(C(NCC2CCCCC2)c2ccccc2)ccc2cccnc12] | A | 7- (((cyclohexylmethyl)amino)(phenyl)methyl)quinolin- 8-ol LC-MS: m/z 347 (M + H)⁺. 1H NMR (400 MHz, CD3OD) δ (ppm): 9.03 (d, J = 4.4 Hz, 1H), 8.75 (d, J = 8.4 Hz, 1H), 7.88 (d, J = 4.8 Hz, 1H), 7.86-7.75 (m, 2H), 7.67 (d, J = 7.6 Hz, 2H), 7.51-7.44 (m, 3H), 7.30-7.19 (m, 5H), 6.18 (s, 1H), 3.30-3.25 (m, 2H), 3.14-3.10 (m, 2H). |
| 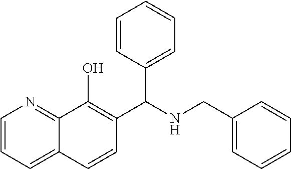 [SMILES: Oc1c(C(NCc2ccccc2)c2ccccc2)ccc2cccnc12] | B | 7-((benzylamino)(phenyl)methyl)quinolin-8-ol LC-MS: m/z 341 (M + H)⁺. 1H NMR (400 MHz, CD3OD) δ (ppm): 9.07-9.05 (m, 1H), 8.85-8.82 (m, 1H), 7.95-7.91 (m, 1H), 7.86-7.80 (m, 2H), 7.66 (d, J = 7.2 Hz, 2H), 7.52- 7.44 (m, 9H), 6.15 (s, 1H), 4.30 (d, J = 3.2 Hz, 2H). |
| 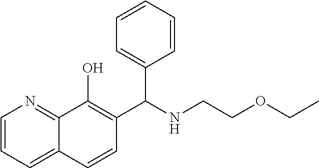 [SMILES: CCOCCNC(c1ccccc1)c1ccc2cccnc2c1O] | A | 7-(((2-ethoxyethyl)amino)(phenyl)methyl)quinolin- 8-ol LC-MS: m/z 323 (M + H)⁺. 1H NMR (400 MHz, CD3OD) δ (ppm): 8.78 (d, J = 3.2 Hz, 1H), 8.20 (d, J = 7.6 Hz, 1H), 7.49-7.44 (m, 3H), 7.34-7.22 (m, 5H), 5.34 (s, 1H), 3.64-3.60 (m, 2H), 3.54-3.48 (m, 2H), 2.90-2.81 (m, 2H), 1.22-1.17 (m, 3H). |
| 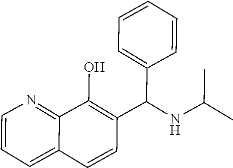 [SMILES: CC(C)NC(c1ccccc1)c1ccc2cccnc2c1O] | B | 7-((isopropylamino)(phenyl)methyl)quinolin-8-ol LC-MS: m/z 293 (M + H)⁺. 1H NMR (400 MHz, DMSO-d6) δ (ppm): 8.82 (d, J = 3.2 Hz, 1H), 8.31 (s, 1H), 8.25 (d, J = 8.0 Hz, 1H), 7.55-7.47 (m, 4H), 7.34-7.17 (m, 4H), 5.5 (s, 1H), 2.70-2.63 (m, 1H), 1.10-1.06 (m, 6H). |
| 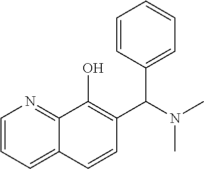 [SMILES: CN(C)C(c1ccccc1)c1ccc2cccnc2c1O] | B | 7-((dimethylamino)(phenyl)methyl)quinolin-8-ol LC-MS: m/z 279 (M + H)⁺. 1H NMR (400 MHz, CD3OD) δ (ppm): 9.01-9.00 (m, 1H), 8.65 (d, J = 8.4 Hz, 1H), 7.84-7.70 (m, 5H), 7.51-7.42 (m, 3H), 5.94 (s, 1H), 3.65 (s, 1H), 3.31-3.30 (s, 6H). |
| 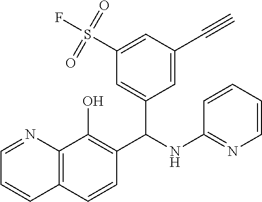 [SMILES: C#Cc1cc(C(Nc2ccccn2)c2ccc3cccnc3c2O)cc(S(=O)(=O)F)c1] | A | 3-ethynyl-5-((8-hydroxyquinolin-7-yl)(pyridin-2- ylamino)methyl)benzenesulfonyl fluoride LC-MS: m/z 434 (M + H)⁺. 1H NMR (400 MHz, DMSO-d6) δ 8.80 (d, J = 4.6 Hz, 1H), 8.25 (d, J = 8.4 Hz, 1H), 7.99 (s, 2H), 7.88 (s, 2H), 7.57-7.54 (m, 2H), 7.52-7.48 (m, 1H), 7.39-7.35 (m, 3H), 6.90 (d, J = 8.2 Hz, 1H), 6.67 (d, J = 8.4 Hz, 1H), 6.52-6.43 (m, 1H), 4.48 (s, 1H). |
| 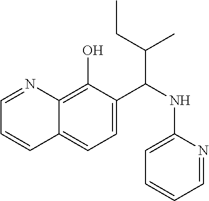 [SMILES: CCC(C)C(Nc1ccccn1)c1ccc2cccnc2c1O] | 2 | 7-(2-methyl-1-(pyridin-2-ylamino)butyl)quinolin- 8-ol LC-MS: m/z 308 (M + H)⁺. 1H NMR (400 MHz, DMSO-d6) δ (ppm): 10.00 (br s, 1H), 8.84 (d, J = 4.0 Hz, 1H), 8.27 (d, J = 8.4 Hz, 1H), 7.86(s, 1H), 7.59-7.49 (m, 2H), 7.36-7.28 (m, 2H), 6.93-6.84 (m, 1H), 6.59-6.52 (m, 1H), 6.40 (t, J = 4.8 Hz, 1H), 5.43-5.25 (m, 1H), 2.02- 1.95 (m, 2H), 1.70-1.30 (m, 2H), 0.96-0.75 (m, 6H). |
| 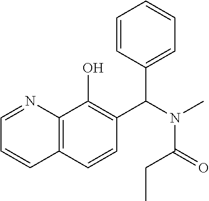 [SMILES: CCC(=O)N(C)C(c1ccccc1)c1ccc2cccnc2c1O] | 6 | N-((8-hydroxyquinolin-7-yl)(phenyl)methyl)-N- methylpropionamide LC-MS: m/z 321 (M + H)⁺. 1H NMR (400 MHz, DMSO-d6) mixture of rotamers δ 8.88 (dd, J = 10.6, 4.1 Hz, 1H), 8.36- 8.33 (m, 1H), 7.62-7.56 (m, 1H), 7.47-7.23 (m, 5H), 7.19-7.02 (m, 3H), 2.80 (s, 2H), 2.64 (s, 1H), 2.48-2.37 (m, 2H), 1.01 (dt, J = 14.5, 7.3 Hz, 3H). |
| 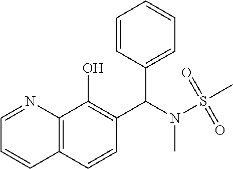 [SMILES: CN(C(c1ccccc1)c1ccc2cccnc2c1O)S(C)(=O)=O] | 6 | N-((8-hydroxyquinolin-7-yl)(phenyl)methyl)-N- methylmethanesulfonamide LC-MS: m/z 343 (M + H)⁺. 1H NMR (400 MHz, DMSO-d6) δ 8.90 (dd, J = 4.2, 1.6 Hz, 1H), 8.43 (s, 1H), 8.36 (d, J = 8.3 Hz, 1H), 7.61 (dd, J = 8.3, 4.2 Hz, 1H), 7.43-7.26 (m, 5H), 7.20 (d, J = 7.6 Hz, 2H), 7.14 (d, J = 8.6 Hz, 1H), 6.81 (s, 1H), 3.01 (s, 3H), 2.69 (s, 3H). |
| 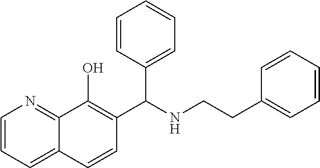 [SMILES: Oc1c(C(NCCc2ccccc2)c2ccccc2)ccc2cccnc12] | 1 | 7-((phenethylamino)(phenyl)methyl)quinolin-8-ol LC-MS: m/z 355 (M + H)⁺. 1H NMR (400 MHz, CD3OD) δ (ppm): 9.03 (d, J = 4.4 Hz, 1H), 8.75 (d, J = 8.4 Hz, 1H), 7.88-7.75 (m, 3H), 7.67 (d, J = 7.6 Hz, 2H), 7.51-7.44 (m, 3H), 7.30-7.19 (m, 5H), 6.18 (s, 1H), 3.30-3.25 (m, 2H), 3.14-3.10 (m, 2H). |
| 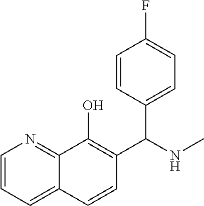 [SMILES: CNC(c1ccc(F)cc1)c1ccc2cccnc2c1O] | 1 | 7-((4-fluorophenyl)(methylamino)methyl)quinolin- 8-ol LC-MS: m/z 283 (M + H)⁺. 1H NMR (400 MHz, DMSO-d6) δ 8.82 (dd, J = 4.2, 1.7 Hz, 1H), 8.30-8.20 (m, 1H), 7.57 (d, J = 8.6 Hz, 1H), 7.52-7.46 (m, 3H), 7.36 (d, J = 8.5 Hz, 1H), 7.14-7.09 (m, 2H), 5.24 (s, 1H), 2.28 (s, 3H). |
| 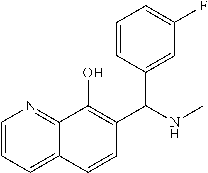 [SMILES: CNC(c1cccc(F)c1)c1ccc2cccnc2c1O] | 1 | 7-((3-fluorophenyl)(methylamino)methyl)quinolin- 8-ol LC-MS: m/z 283 (M + H)⁺. 1H NMR (400 MHz, CD3OD) δ (ppm): 9.21 (d, J = 4.8 Hz, 1H), 9.00 (d, J = 8.4 Hz, 1H), 8.05-8.01 (m, 1H), 7.91 (s, 2H), 7.54-7.42 (m, 3H), 7.22 (t, J = 8.4 Hz, 1H), 6.19 (s, 1H), 2.80 (s, 3H). |
| 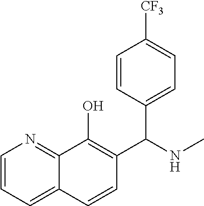 [SMILES: CNC(c1ccc(C(F)(F)F)cc1)c1ccc2cccnc2c1O] | 1 | 7-((methylamino)(4- (trifluoromethyl)phenyl)methyl)quinolin-8-ol LC-MS: m/z 333 (M + H)⁺. 1H NMR (400 MHz, CD3OD) δ (ppm): 8.81 (s, 1H), 8.25 (d, J = 8.4 Hz, 1H), 7.72-7.62 (m, 4H), 7.51-7.35 (m, 3H), 5.52 (s, 1H), 2.56 (s, 3H). |
| 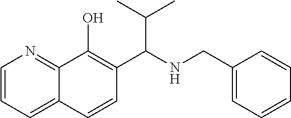 [SMILES: CC(C)C(NCc1ccccc1)c1ccc2cccnc2c1O] | 1 | 7-(1-(benzylamino)-2-methylpropyl)quinolin-8-ol LC-MS: m/z 307 (M + H)⁺. 1H NMR (400 MHz, CD3OD) δ (ppm): 8.78 (d, J = 4.0 Hz, 1H), 8.24 (d, J = 8.4 Hz, 1H), 7.48-7.45 (m, 1H), 7.33-7.19 (m, 7H), 3.83-3.77 (m, 2H), 3.59 (d, J = 13.2 Hz, 1H), 2.14-2.08 (m, 2H), 1.00 (d, J = 6.8 Hz, 3H), 0.85(d, J = 6.8 Hz, 3H). |
| 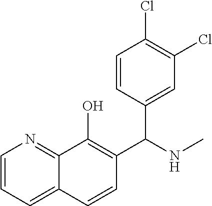 [SMILES: CNC(c1ccc(Cl)c(Cl)c1)c1ccc2cccnc2c1O] | 1 | 7-((3,4- dichlorophenyl)(methylamino)methyl)quinolin-8-ol LC-MS: m/z 333, 335 (M + H)⁺. 1H NMR (400 MHz, CD3OD) δ (ppm): 8.79 (d, J = 4.0 Hz, 1H), 8.22 (d, J = 8.4 Hz, 1H), 7.66 (s, 1H), 7.49-7.34 (m, 5H), 5.23 (s, 1H), 2.43 (s, 3H). |
| 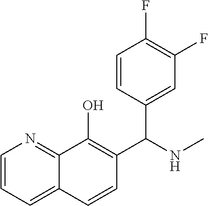 [SMILES: CNC(c1ccc(F)c(F)c1)c1ccc2cccnc2c1O] | 1 | 7-((3,4- difluorophenyl)(methylamino)methyl)quinolin-8-ol LC-MS: m/z 301 (M + H)⁺. 1H NMR (400 MHz, CD3OD) δ (ppm): 8.79 (d, J = 4.0 Hz, 1H), 8.22 (d, J = 8.4 Hz, 1H), 7.49-7.46 (m, 1H), 7.43-7.33 (m, 3H), 7.27-7.17 (m, 2H), 5.22 (s, 1H), 2.44 (s, 3H). |
| 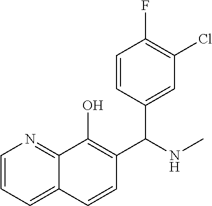 [SMILES: CNC(c1ccc(F)c(Cl)c1)c1ccc2cccnc2c1O] | 1 | 7-((3-chloro-4- fluorophenyl)(methylamino)methyl)quinolin-8-ol LC-MS: m/z 317 (M + H)⁺. 1H NMR (400 MHz, CD3OD) δ (ppm): 8.79 (d, J = 4.4 Hz, 1H), 8.22 (d, J = 8.4 Hz, 1H), 7.61(d, J = 6.8 Hz, 1H), 7.49-7.33 (m, 4H), 7.19 (t, J = 9.2 Hz, 1H), 5.22 (s, 1H), 2.43 (s, 3H). |
| 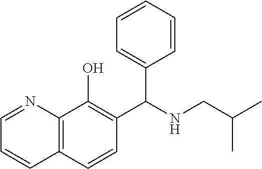 [SMILES: CC(C)CNC(c1ccccc1)c1ccc2cccnc2c1O] | 1 | 7-((isobutylamino)(phenyl)methyl)quinolin-8-ol LC-MS: m/z 307 (M + H)⁺. 1H NMR (400 MHz, DMSO-d6) δ 8.81 (dd, J = 4.1, 1.6 Hz, 1H), 8.24 (dd, J = 8.2, 1.7 Hz, 1H), 7.58-7.43 (m, 5H), 7.38-7.25 (m, 4H), 7.21- 7.17 (m, 1H), 5.32 (s, 1H), 2.36 (dd, J = 11.3, 6.4 Hz, 1H), 2.27 (dd, J = 11.3, 7.1 Hz, 1H), 1.77 (dt, J = 13.5, 6.7 Hz, 1H), 0.89 (dd, J = 8.5, 6.6 Hz, 6H). |
| 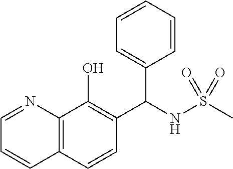 [SMILES: CS(=O)(=O)NC(c1ccccc1)c1ccc2cccnc2c1O] | 6 | N-((8-hydroxyquinolin-7- yl)(phenyl)methyl)methanesulfonamide LC-MS: m/z 329 (M + H)⁺. 1H NMR (400 MHz, DMSO-d6) δ 8.80 (dd, J = 4.2, 1.7 Hz, 1H), 8.24 (dd, J = 8.4, 1.7 Hz, 1H), 7.58 (d, J = 8.6 Hz, 1H), 7.49 (dd, J = 8.3, 4.2 Hz, 1H), 7.41-7.21 (m, 6H), 7.17-7.14 (m, 1H), 6.18 (s, 1H), 2.61 (s, 3H). |
| 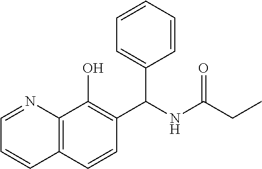 [SMILES: CCC(=O)NC(c1ccccc1)c1ccc2cccnc2c1O] | 6 | N-((8-hydroxyquinolin-7- yl)(phenyl)methyl)propionamide LC-MS: m/z 307 (M + H)⁺. 1H NMR (400 MHz, DMSO-d6) δ 9.95 (s, 1H), 8.89-8.82 (m, 1H), 8.70 (d, J = 8.8 Hz, 1H), 8.34- 8.26 (m, 1H), 7.57-7.54 (m, 2H), 7.43 (d, J = 8.6 Hz, 1H), 7.32-7.19 (m, 5H), 6.72 (d, J = 8.7 Hz, 1H), 2.24 (qd, J = 7.5, 4.5 Hz, 2H), 1.02 (t, J = 7.6 Hz, 3H). |
| 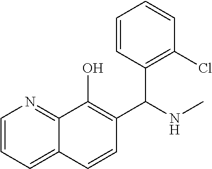 [SMILES: CNC(c1ccccc1Cl)c1ccc2cccnc2c1O] | 1 | 7-((2-chlorophenyl)(methylamino)methyl)quinolin- 8-ol LC-MS: m/z 299 (M + H)⁺. 1H NMR (400 MHz, CD3OD) δ (ppm): 9.06 (d, J = 4.8 Hz, 1H), 8.78 (d, J = 8.4 Hz, 1H), 7.92-7.86 (m, 2H), 7.72(d, J = 8.4 Hz, 1H), 7.59-7.46 (m, 4H), 6.42 (s, 1H), 2.83 (s, 3H). |
| 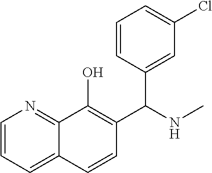 [SMILES: CNC(c1cccc(Cl)c1)c1ccc2cccnc2c1O] | 1 | 7-((3-chlorophenyl)(methylamino)methyl)quinolin- 8-ol LC-MS: m/z 299 (M + H)⁺. 1H NMR (400 MHz, CD3OD) δ (ppm): 8.91 (d, J = 4.0 Hz, 1H), 8.31 (d, J = 8.4 Hz, 1H), 7.70 (s, 1H), 7.61-7.56 (m, 2H), 7.49-7.44 (m, 4H), 5.85 (s, 1H), 2.76 (s, 3H). |
| 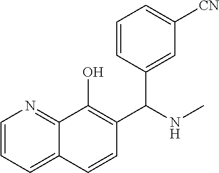 [SMILES: CNC(c1cccc(C#N)c1)c1ccc2cccnc2c1O] | 1 | 3-((8-hydroxyquinolin-7- yl)(methylamino)methyl)benzonitrile LC-MS: m/z 290 (M + H)⁺. 1H NMR (400 MHz, CD3OD) δ (ppm): 8.87 (d, J = 3.2 Hz, 1H), 8.28 (d, J = 8.4 Hz, 1H), 7.98 (s, 1H), 7.90 (d, J = 7.6 Hz, 1H), 7.73 (d, J = 7.6 Hz, 1H), 7.61-7.47 (m, 4H), 5.70 (s, 1H), 2.66 (s, 3H). |
| 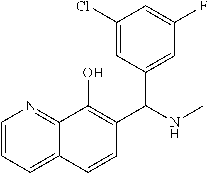 [SMILES: CNC(c1cc(F)cc(Cl)c1)c1ccc2cccnc2c1O] | 1 | 7-((3-chloro-5- fluorophenyl)(methylamino)methyl)quinolin-8-ol LC-MS: m/z 317 (M + H)⁺. 1H NMR (400 MHz, CD3OD) δ (ppm): 8.79 (s, 1H), 8.23 (d, J = 7.2 Hz, 1H), 7.47-7.28 (m, 6H), 5.27 (s, 1H), 2.45 (s, 3H). |
| 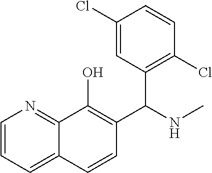 [SMILES: CNC(c1cc(Cl)ccc1Cl)c1ccc2cccnc2c1O] | 1 | 7-((2,5- dichlorophenyl)(methylamino)methyl)quinolin-8-ol LC-MS: m/z 333, 335 (M + H)⁺. 1H NMR (400 MHz, CD3OD) δ (ppm): 9.10 (d, J = 4.0 Hz, 1H), 8.96-9.86 (m, 1H), 7.97 (s, 2H), 7.79 (d, J = 8.4 Hz, 1H), 7.66 (d, J = 8.4 Hz, 1H), 7.56-7.50 (m, 2H), 6.40 (s, 1H), 2.84 (s, 3H). |
| 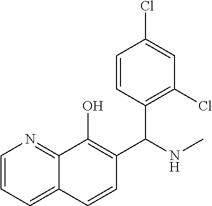 [SMILES: CNC(c1ccc(Cl)cc1Cl)c1ccc2cccnc2c1O] | 1 | 7-((2,4- dichlorophenyl)(methylamino)methyl)quinolin-8-ol LC-MS: m/z 333, 335 (M + H)⁺. 1H NMR (400 MHz, CD3OD) δ (ppm): 9.07 (d, J = 4.8 Hz, 1H), 8.82 (d, J = 8.0 Hz, 1H), 7.94 (d, J = 3.2 Hz, 1H), 7.87 (d, J = 8.4 Hz, 1H), 7.76 (d, J = 8.8 Hz, 1H), 7.65-7.58 (m, 3H), 6.39 (s, 1H), 2.83 (s, 3H). |
| 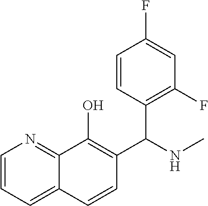 [SMILES: CNC(c1ccc(F)cc1F)c1ccc2cccnc2c1O] | 1 | 7-((2,4- difluorophenyl)(methylamino)methyl)quinolin-8-ol LC-MS: m/z 301 (M + H)⁺. 1H NMR (400 MHz, CD3OD) δ (ppm): 9.01 (d, J = 4.8 Hz, 1H), 8.64 (d, J = 8.4 Hz, 1H), 7.82-7.66 (m, 4H), 7.18-7.11 (m, 2H), 6.23 (s, 1H), 2.80 (s, 3H). |
| 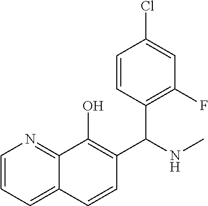 [SMILES: CNC(c1ccc(Cl)cc1F)c1ccc2cccnc2c1O] | 1 | 7-((4-chloro-2- fluorophenyl)(methylamino)methyl)quinolin-8-ol LC-MS: m/z 317 (M + H)⁺. 1H NMR (400 MHz, CD3OD) δ (ppm): 8.92 (d, J = 4.4 Hz, 1H), 8.36 (d, J = 8.0 Hz, 1H), 7.76-7.72 (m, 1H), 7.63-7.60 (m, 1H), 7.52 (s, 2H), 7.39 (t, J = 8.4 Hz, 2H), 6.14 (s, 1H), 2.79 (s, 3H). |
| 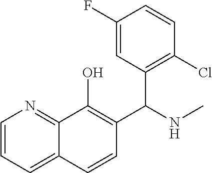 [SMILES: CNC(c1cc(F)ccc1Cl)c1ccc2cccnc2c1O] | 1 | 7-((2-chloro-5- fluorophenyl)(methylamino)methyl)quinolin-8-ol LC-MS: m/z 317 (M + H)⁺. 1H NMR (400 MHz, CD3OD) δ (ppm): 8.80 (s, 1H), 8.23 (d, J = 8.0 Hz, 1H), 7.48-7.23 (m 5H), 7.07 (t, J = 7.2 Hz, 1H), 5.65 (s, 1H), 2.50 (s, 3H). |
| 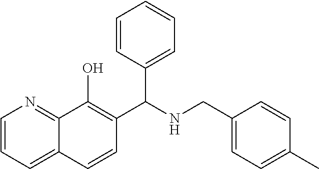 | 1 | 7-(((4- methylbenzyl)amino)(phenyl)methyl)quinolin-8-ol LC-MS: m/z 341 (M + H)⁺. 1H NMR (400 MHz, CD3OD) δ (ppm): 8.78 (s, 1H), 8.21 (s, 1H), 7.50-7.42 (m, 3H), 7.32-7.21 (m, 7H), 7.15 (d, J = 7.2 Hz, 2H), 5.25 (s, 1H), 3.85- 3.75 (m, 2H), 2.31 (s, 3H). |
| 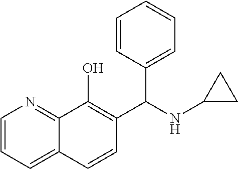 [SMILES: Oc1c(C(NC2CC2)c2ccccc2)ccc2cccnc12] | 1 | 7-((cyclopropylamino)(phenyl)methyl)quinolin-8- ol LC-MS: m/z 291 (M + H)⁺. 1H NMR (400 MHz, CD3OD) δ (ppm): 8.78 (s, 1H), 8.20 (d, J = 8.0 Hz, 1H), 7.52-7.44 (m, 3H), 7.37-7.23 (m, 5H), 5.50 (s, 1H), 2.25 (d, J = 3.6 Hz, 1H), 0.59-0.48 (m, 4H). |
| 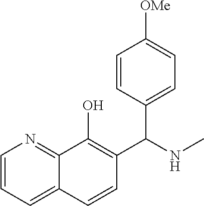 [SMILES: CNC(c1ccc(OC)cc1)c1ccc2cccnc2c1O] | 1 | 7-((4- methoxyphenyl)(methylamino)methyl)quinolin-8- ol LC-MS: m/z 295 (M + H)⁺. 1H NMR (400 MHz, DMSO-d6) δ 8.81 (dd, J = 4.2, 1.8 Hz, 1H), 8.24 (dd, J = 8.4, 1.7 Hz, 1H), 7.55-7.45 (m, 2H), 7.36-7.32 (m, 3H), 6.89- 6.80 (m, 2H), 5.15 (s, 1H), 3.69 (s, 3H), 2.29 (s, 3H). |
| 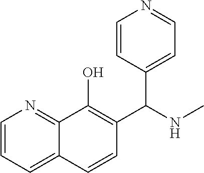 | 1 | 7-((methylamino)(pyridin-4-yl)methyl)quinolin-8- ol LC-MS: m/z 295 (M + H)⁺. 1H NMR (400 MHz, DMSO-d6) δ 8.84 (dd, J = 4.2, 1.7 Hz, 1H), 8.51-8.43 (m, 2H), 8.28 (dd, J = 8.3, 1.7 Hz, 1H), 8.20 (s, 1H), 7.60 (d, J = 8.5 Hz, 1H), 7.53 (dd, J = 8.3, 4.2 Hz, 1H), 7.48-7.43 (m, 2H), 7.39 (d, J = 8.5 Hz, 1H), 5.29 (s, 1H), 2.29 (s, 3H). |
| 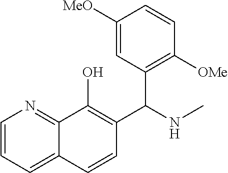 | 1 | 7-((2,5- dimethoxyphenyl)(methylamino)methyl)quinolin- 8-ol LC-MS: m/z 295 (M + H)⁺. 1H NMR (400 MHz, DMSO-d6) δ 8.74 (dd, J = 4.1, 1.8 Hz, 1H), 8.16 (dd, J = 8.2, 1.8 Hz, 1H), 7.42 (dd, J = 8.2, 4.1 Hz, 1H), 7.22-7.17 (m, 2H), 6.90-6.80 (m, 2H), 6.73 (dd, J = 8.8, 3.0 Hz, 1H), 5.35 (s, 1H), 3.65 (s, 3H), 3.58 (s, 3H), 2.25 (s, 3H). |
| 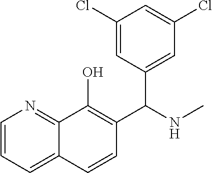 [SMILES: CNC(c1cc(Cl)cc(Cl)c1)c1ccc2cccnc2c1O] | 1 | 7-((3,5- dichlorophenyl)(methylamino)methyl)quinolin-8-ol LC-MS: m/z 333, 335 (M + H)⁺. 1H NMR (400 MHz, CD3OD) δ (ppm): 9.07 (d, J = 4.4 Hz, 1H), 8.80 (d, J = 8.4 Hz, 1H), 7.92-7.89 (m, 1H), 7.83-7.76 (m, 2H), 7.67 (s, 2H), 7.56 (s, 1H), 6.07(s, 1H), 2.79 (s, 3H). |
| 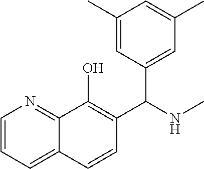 [SMILES: CNC(c1cc(C)cc(C)c1)c1ccc2cccnc2c1O] | 1 | 7-((3,5- dimethylphenyl)(methylamino)methyl)quinolin-8- ol LC-MS: m/z 293 (M + H)⁺. 1H NMR (400 MHz, CD3OD) δ (ppm): 9.06 (d, J = 4.8 Hz, 1H), 8.84 (d, J = 8.4 Hz, 1H), 7.94-7.91 (m, 1H), 7.82-7.75 (m, 2H), 7.21 (s, 2H), 7.09 (s, 1H), 5.97 (s, 1H), 2.75 (s, 3H), 2.32 (s, 3H), 2.31 (s, 3H) |
| 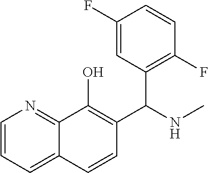 [SMILES: CNC(c1cc(F)ccc1F)c1ccc2cccnc2c1O] | 1 | 7-((2,5- difluorophenyl)(methylamino)methyl)quinolin-8-ol LC-MS: m/z 301 (M + H)⁺. 1H NMR (400 MHz, DMSO-d6) δ (ppm): 10.42 (br s, 2H), 8.96 (d, J = 4.0 Hz, 1H), 8.48 (d, J = 8.0 Hz, 1H), 7.96 (t, J = 5.6 Hz, 1H), 7.88 (d, J = 8.8 Hz, 1H), 7.71-7.68 (m, 1H), 7.57 (d, J = 8.8 Hz, 1H), 7.35-7.32 (m, 2H), 6.23 (s, 1H), 2.59 (s, 3H). |
| 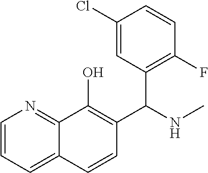 [SMILES: CNC(c1cc(Cl)ccc1F)c1ccc2cccnc2c1O] | 1 | 7-((5-chloro-2- fluorophenyl)(methylamino)methyl)quinolin-8-ol LC-MS: m/z 317 (M + H)⁺. 1H NMR (400 MHz, CD3OD) δ (ppm): 8.79 (s, 1H), 8.23 (d, J = 8.0 Hz, 1H), 7.57 (d, J = 4.8 Hz, 1H), 7.49 (d, J = 3.6 Hz, 1H), 7.36-7.28 (m, 3H), 7.13 (t, J = 9.2 Hz, 1H), 5.55(s, 1H) 2.03 (s, 3H). |
| 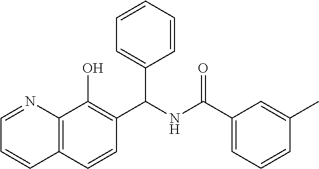 [SMILES: Cc1cccc(C(=O)NC(c2ccccc2)c2ccc3cccnc3c2O)c1] | 4 | N-((8-hydroxyquinolin-7-yl)(phenyl)methyl)-3- methylbenzamide LC-MS: m/z 369 (M + H)⁺. 1H NMR (400 MHz, DMSO-d6) δ 10.06 (brs, 1H), 9.20 (d, J = 8.7 Hz, 1H), 8.87 (dd, J = 4.2, 1.8 Hz, 1H), 8.32 (dd, J = 8.3, 1.9 Hz, 1H), 7.79-7.65 (m, 3H), 7.56 (dd, J = 8.4, 4.1 Hz, 1H), 7.44 (d, J = 8.6 Hz, 1H), 7.40-7.29 (m, 6H), 7.29-7.18 (m, 1H), 6.99 (d, J = 8.6 Hz, 1H), 2.36 (s, 3H). |
| 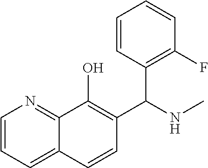 [SMILES: CNC(c1ccccc1F)c1ccc2cccnc2c1O] | 1 | 7-((2-fluorophenyl)(methylamino)methyl)quinolin- 8-ol LC-MS: m/z 283 (M + H)⁺. 1H NMR (400 MHz, CD3OD) δ (ppm): 9.12 (d, J = 4.8 Hz, 1H), 8.98 (d, J = 8.4 Hz, 1H), 8.04-8.01 (m, 1H), 7.90-7.84 (m, 2H), 7.77 (t, J = 8.0 Hz, 1H), 7.574-7.49 (m, 1H), 7.38-7.24 (m, 2H), 6.40 (s, 1H), 2.82 (s, 3H). |
| 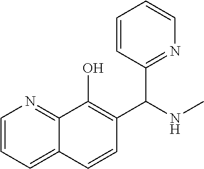 [SMILES: CNC(c1ccccn1)c1ccc2cccnc2c1O] | 1 | 7-((methylamino)(pyridin-2-yl)methyl)quinolin-8- ol LC-MS: m/z 266 (M + H)⁺. 1H NMR (400 MHz, CD3OD) δ (ppm): 9.20-9.15 (m, 2H), 8.82 (d, J = 4.8 Hz, 1H), 8.18-8.15 (m, 1H), 8.01 (t, J = 8.0 Hz, 1H), 7.92 (s, 2H), 7.69 (d, J = 7.6 Hz, 1H), 7.60 (t, J = 6.0 Hz, 1H), 6.31 (s, 1H), 2.74 (s, 3H). |
| 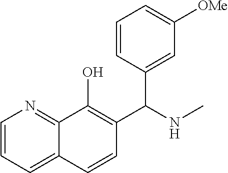 [SMILES: CNC(c1cccc(OC)c1)c1ccc2cccnc2c1O] | 1 | 7-((3- methoxyphenyl)(methylamino)methyl)quinolin-8- ol LC-MS: m/z 295 (M + H)⁺. 1H NMR (400 MHz, CD3OD) δ (ppm): 8.92 (d, J = 4.4 Hz, 1H), 8.36 (d, J = 8.4 Hz, 1H), 7.63-7.61 (m, 1H), 7.50 (s, 2H), 7.40 (t, J = 8.0 Hz, 1H), 7.22-7.16 (m, 2H), 6.99 (d, J = 8.0 Hz, 1H), 5.84 (s, 1H), 3.81 (s, 3H), 2.75 (s, 3H). |
| 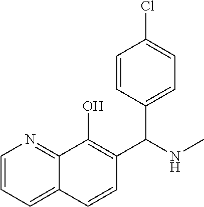 [SMILES: CNC(c1ccc(Cl)cc1)c1ccc2cccnc2c1O] | 1 | 7-((4-chlorophenyl)(methylamino)methyl)quinolin- 8-ol LC-MS: m/z 299 (M + H)⁺. 1H NMR (400 MHz, CD3OD) δ (ppm): 8.88 (d, J = 3.2 Hz, 1H), 8.28 (d, J = 8.4 Hz, 1H), 7.61-7.54 (m, 3H), 7.46-7.42 (m, 4H), 5.75 (s, 1H), 2.71 (s, 3H) |
| 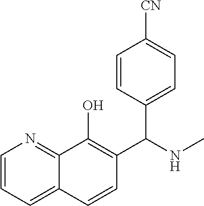 [SMILES: CNC(c1ccc(C#N)cc1)c1ccc2cccnc2c1O] | 1 | 4-((8-hydroxyquinolin-7- yl)(methylamino)methyl)benzonitrile LC-MS: m/z 290 (M + H)⁺. 1H NMR (400 MHz, CD3OD) δ (ppm): 9.09 (d, J = 4.4 Hz, 1H), 8.89 (d, J = 8.0 Hz, 1H), 7.98 (t, J = 6.8 Hz, 1H), 7.87-7.84 (m, 6H), 6.25 (s, 1H), 2.80 (s, 3H) |
| 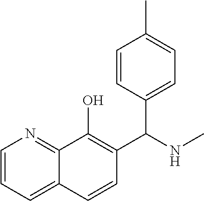 [SMILES: CNC(c1ccc(C)cc1)c1ccc2cccnc2c1O] | 1 | 7-((methylamino)(p-tolyl)methyl)quinolin-8-ol LC-MS: m/z 279 (M + H)⁺. 1H NMR (400 MHz, CD3OD) δ (ppm): 9.07 (d, J = 5.2 Hz, 1H), 8.86 (d, J = 8.4 Hz, 1H), 7.95 (t, J = 6.8 Hz, 1H), 7.83-7.78 (m, 2H), 7.50 (d, J = 7.2 Hz, 2H), 7.31 (d, J = 8.0 Hz, 2H), 6.02 (s, 1H), 2.76 (s, 3H), 2.35 (s, 3H) |
| 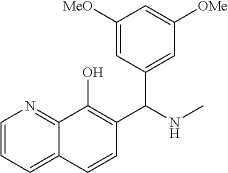 [SMILES: CNC(c1cc(OC)cc(OC)c1)c1ccc2cccnc2c1O] | 1 | 7-((3,5- dimethoxyphenyl)(methylamino)methyl)quinolin- 8-ol LC-MS: m/z 325 (M + H)⁺. 1H NMR (400 MHz, CD3OD) δ (ppm): 9.09 (d, J = 4.8 Hz, 1H), 8.89 (d, J = 8.4 Hz, 1H), 7.98-7.95 (m, 1H), 7.84 (s, 2H), 6.80 (s, 2H), 6.54 (s, 1H), 6.02 (s, 1H), 3.80 (s, 6H), 2.78 (s, 3H) |
| 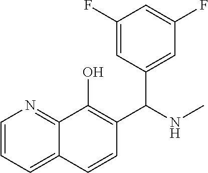 [SMILES: CNC(c1cc(F)cc(F)c1)c1ccc2cccnc2c1O] | 1 | 7-((3,5- difluorophenyl)(methylamino)methyl)quinolin-8-ol LC-MS: m/z 301 (M + H)⁺. 1H NMR (400 MHz, CD3OD) δ (ppm): 9.11 (d, J = 4.4 Hz, 1H), 8.93 (d, J = 8.4 Hz, 1H), 8.01-7.98 (m, 1H), 7.90-7.85 (m, 2H), 7.33 (d, J = 7.2 Hz, 2H), 7.11-7.07 (m, 1H), 6.17 (s, 1H), 2.80 (s, 3H) |
| 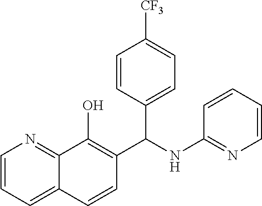 [SMILES: Oc1c(C(Nc2ccccn2)c2ccc(C(F)(F)F)cc2)ccc2cccnc12] | 2 | 7-((pyridin-2-ylamino)(4- (trifluoromethyl)phenyl)methyl)quinolin-8-ol LC-MS: m/z 396 (M + H)⁺. 1H NMR (400 MHz, DMSO-d6) δ (ppm): 10.04 (br s, 1H), 8.87 (t, J = 2.0 Hz, 1H), 8.31 (d, J = 8.0 Hz, 1H), 7.93 (d, J = 4.8 Hz, 1H), 7.67 (d, J = 6.4 Hz, 2H), 7.59-7.53 (m, 4H), 7.48-7.38 (m, 3H), 6.96 (d, J = 8.4 Hz, 1H), 6.72 (d, J = 8.4 Hz, 1H), 6.51 (t, J = 6.0 Hz, 1H) |
| 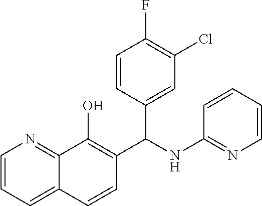 [SMILES: Oc1c(C(Nc2ccccn2)c2ccc(F)c(Cl)c2)ccc2cccnc12] | 2 | 7-((3-chloro-4-fluorophenyl)(pyridin-2- ylamino)methyl)quinolin-8-ol LC-MS: m/z 380 (M + H)⁺. 1H NMR (400 MHz, DMSO-d6) δ (ppm): 10.05 (br s, 1H), 8.86 (t, J = 2.0 Hz, 1H), 8.31 (d, J = 8.4 Hz, 1H), 7.93 (d, J = 4.8 Hz, 1H), 7.61-7.33 (m, 8H), 6.86 (d, J = 8.0 Hz, 1H), 6.70 (d, J = 8.4 Hz, 1H), 6.51 (t, 5.6 Hz, 1H) |
| 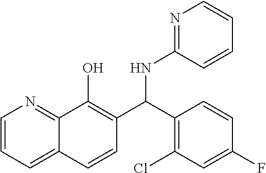 [SMILES: Oc1c(C(Nc2ccccn2)c2ccc(F)cc2Cl)ccc2cccnc12] | 2 | 7-((2-chloro-4-fluorophenyl)(pyridin-2- ylamino)methyl)quinolin-8-ol LC-MS: m/z 380 (M + H)⁺. 1H NMR (400 MHz, DMSO-d6) δ (ppm): 9.95 (br s, 1H), 8.84 (d, J = 3.6 Hz, 1H), 8.46 (d, J = 8.4 Hz, 1H), 8.01-7.98 (m, 2H), 7.68-7.65 (m, 1H), 7.51- 7.22 (m, 5H), 7.16 (t, J = 8.0 Hz, 1H), 6.98 (t, J = 6.4 Hz, 1H), 6.80 (d, J = 4.0 Hz, 1H) |
| 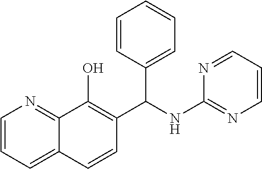 [SMILES: Oc1c(C(Nc2ncccn2)c2ccccc2)ccc2cccnc12] | 3 | 7-(phenyl(pyrimidin-2-ylamino)methyl)quinolin-8- ol LC-MS: m/z 329 (M + H)⁺. 1H NMR (400 MHz, DMSO-d6) δ (ppm): 9.92 (br s, 1H), 8.84 (s, 1H), 8.33-8.28 (m, 3H), 8.08 (d, J = 8.8 Hz, 1H), 7.75 (d, J = 8.0 Hz, 1H), 7.55-7.52 (m, 1H), 7.40-7.18 (m, 6H), 6.99 (d, J = 9.2 Hz, 1H), 6.60 (t, 4.4 Hz, 1H) |
| 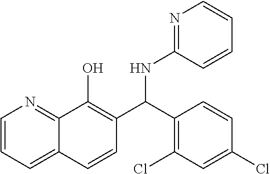 [SMILES: Oc1c(C(Nc2ccccn2)c2ccc(Cl)cc2Cl)ccc2cccnc12] | 2 | 7-((2,4-dichlorophenyl)(pyridin-2- ylamino)methyl)quinolin-8-ol LC-MS: m/z 396, 398 (M + H)⁺. 1H NMR (400 MHz, DMSO-d6) δ (ppm): 9.43 (br s, 1H), 8.93 (d, J = 4.0 Hz, 1H), 8.43 (d, J = 8.4 Hz, 1H), 8.01-7.95 (m, 2H), 7.66-7.47 (m, 4H), 7.38 (s, 1H), 7.25 (d, J = 8.4 Hz, 1H), 7.17-7.17 (m, 1H), 6.99 (t, 6.4 Hz, 1H), 6.74 (d, J = 7.6 Hz, 1H) |
| 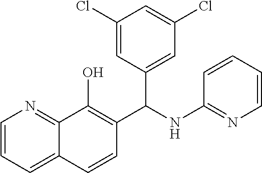 [SMILES: Oc1c(C(Nc2ccccn2)c2cc(Cl)cc(Cl)c2)ccc2cccnc12] | 2 | 7-((3,5-dichlorophenyl)(pyridin-2- ylamino)methyl)quinolin-8-ol LC-MS: m/z 396, 398 (M + H)⁺. 1H NMR (400 MHz, DMSO-d6) δ (ppm): 9.69 (s, 1H), 8.95 (d, J = 3.8 Hz, 1H), 8.48 (d, J = 8.3 Hz, 1H), 8.09-7.96 (m, 2H), 7.69 (dd, J = 7.8, 4.1 Hz, 1H), 7.60 (s, 1H), 7.54 (d, J = 11.2 Hz, 4H), 7.27 (d, J = 8.8 Hz, 1H), 6.97 (t, J = 6.5 Hz, 1H), 6.82 (d, J = 7.3 Hz, 1H). |
| 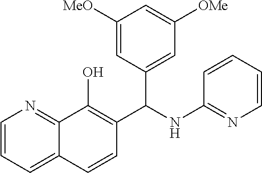 [SMILES: COc1cc(OC)cc(C(Nc2ccccn2)c2ccc3cccnc3c2O)c1] | 2 | 7-((3,5-dimethoxyphenyl)(pyridin-2- ylamino)methyl)quinolin-8-ol LC-MS: m/z 388 (M + H)⁺. 1H NMR (400 MHz, DMSO-d6) δ (ppm): 9.78 (s, 1H), 8.99 (d, J = 4.1 Hz, 1H), 8.61 (d, J = 8.1 Hz, 1H), 8.08-8.06 (s, 1H), 8.00 (t, J = 8.0 Hz, 1H), 7.82-7.71 (m, 1H), 7.60 (s, 2H), 7.29 (d, J = 7.5 Hz, 1H), 6.95 (t, J = 6.5 Hz, 1H), 6.80 (d, J = 6.5 Hz, 1H), 6.69 (s, 2H), 6.47 (s, 1H), 3.73 (s, 6H). |
| 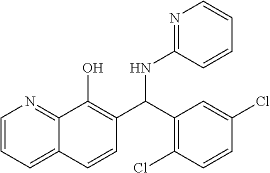 [SMILES: Oc1c(C(Nc2ccccn2)c2cc(Cl)ccc2Cl)ccc2cccnc12] | 2 | 7-((2,5-dichlorophenyl)(pyridin-2- ylamino)methyl)quinolin-8-ol LC-MS: m/z 396, 398 (M + H)⁺. 1H NMR (400 MHz, DMSO-d6) δ (ppm): 9.26 (br s, 1H), 8.94-8.85 (m, 1H), 8.39 (d, J = 8.2 Hz, 1H), 8.05-7.85 (m, 2H), 7.69-7.56 (m, 2H), 7.53-7.42 (m, 2H), 7.38 (s, 1H), 7.23 (d, J = 8.3 Hz, 1H), 7.12 (d, J = 7.0 Hz, 1H), 6.98-6.87 (m, 1H), 6.74 (d, J = 7.0 Hz, 1H) |
| 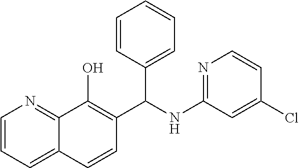 [SMILES: Oc1c(C(Nc2cc(Cl)ccn2)c2ccccc2)ccc2cccnc12] | 2 | 7-(((4-chloropyridin-2- yl)amino)(phenyl)methyl)quinolin-8-ol LC-MS: m/z 362 (M + H)⁺. 1H NMR (400 MHz, DMSO-d6) δ (ppm): 9.96 (br s, 1H), 8.85 (s, 1H), 8.29 (d, J = 8.1 Hz, 1H), 7.89 (d, J = 5.3 Hz, 1H), 7.69 (d, J = 8.1 Hz, 1H), 7.60- 7.49 (m, 2H), 7.40 (d, J = 8.4 Hz, 1H), 7.37-7.25 (m, 4H), 7.24-7.15 (m, 1H), 6.85 (d, J = 8.0 Hz, 1H), 6.76 (s, 1H), 6.55 (d, J = 5.0 Hz, 1H). |
| 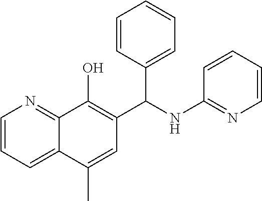 [SMILES: Cc1cc(C(Nc2ccccn2)c2ccccc2)c(O)c2ncccc12] | 2 | 5-methyl-7-(phenyl(pyridin-2- ylamino)methyl)quinolin-8-ol LC-MS: m/z 342 (M + H)⁺. 1H NMR (400 MHz, CD3OD) δ (ppm): 8.86 (s, 1H), 8.43 (d, J = 8.6 Hz, 1H), 7.92 (t, J = 8.0 Hz, 1H), 7.87 (d, J = 5.9 Hz, 1H), 7.59 (dd, J = 7.7, 3.8 Hz, 1H), 7.48-7.38 (m, 4H), 7.35 (d, J = 6.8 Hz, 1H), 7.20-7.09 (m, 2H), 6.92 (t, J = 6.6 Hz, 1H), 6.61 (s, 1H), 2.54 (s, 3H). |
| 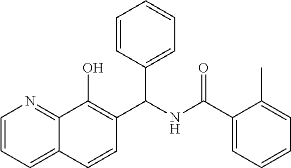 [SMILES: Cc1ccccc1C(=O)NC(c1ccccc1)c1ccc2cccnc2c1O] | 4 | N-((8-hydroxyquinolin-7-yl)(phenyl)methyl)-2- methylbenzamide LC-MS: m/z 369 (M + H)⁺. 1H NMR (400 MHz, DMSO-d6) δ (ppm): 10.03 (br s, 1H), 9.21 (d, J = 9.0 Hz, 1H), 8.87 (dd, J = 4.1, 1.5 Hz, 1H), 8.32 (dd, J = 8.3, 1.5 Hz, 1H), 7.67 (d, J = 8.6 Hz, 1H), 7.56 (dd, J = 8.3, 4.2 Hz, 1H), 7.43 (d, J = 8.6 Hz, 1H), 7.39-7.28 (m, 6H), 7.27-7.18 (m, 3H), 6.97 (d, J = 9.0 Hz, 1H), 2.28 (s, 3H). |
| 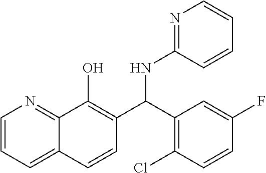 [SMILES: Oc1c(C(Nc2ccccn2)c2cc(F)ccc2Cl)ccc2cccnc12] | 2 | 7-((2-chloro-5-fluorophenyl)(pyridin-2- ylamino)methyl)quinolin-8-ol LC-MS: m/z 380 (M + H)⁺. 1H NMR (400 MHz, DMSO-d6) δ (ppm): 9.62 (s, 1H), 8.95 (d, J = 3.7 Hz, 1H), 8.50 (d, J = 7.4 Hz, 1H), 8.11-7.95 (m, 2H), 7.74-7.66 (m, 1H), 7.65- 7.57 (m, 1H), 7.51 (d, J = 8.6 Hz, 1H), 7.40-7.12 (m, 4H), 6.99 (t, J = 6.6 Hz, 1H), 6.75 (d, J = 6.7 Hz, 1H). |
| 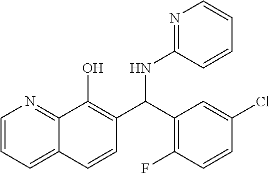 [SMILES: Oc1c(C(Nc2ccccn2)c2cc(Cl)ccc2F)ccc2cccnc12] | 2 | 7-((5-chloro-2-fluorophenyl)(pyridin-2- ylamino)methyl)quinolin-8-ol LC-MS: m/z 380 (M + H)⁺. 1H NMR (400 MHz, DMSO-d6) δ (ppm): 9.36 (s, 1H), 8.91 (d, J = 2.8 Hz, 1H), 8.39 (d, J = 8.3 Hz, 1H), 8.06-7.88 (m, 2H), 7.63 (dd, J = 8.1, 4.1 Hz, 1H), 7.54-7.41 (m, 3H), 7.40-7.28 (m, 2H), 7.20 (d, J = 8.4 Hz, 1H), 6.94 (t, J = 6.2 Hz, 1H), 6.82 (d, J = 6.7 Hz, 1H). |
| 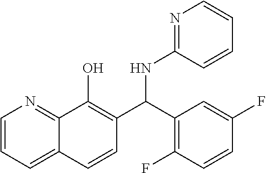 [SMILES: Oc1c(C(Nc2ccccn2)c2cc(F)ccc2F)ccc2cccnc12] | 2 | 7-((2,5-difluorophenyl)(pyridin-2- ylamino)methyl)quinolin-8-ol LC-MS: m/z 364 (M + H)⁺. 1H NMR (400 MHz, DMSO-d6) δ (ppm): 9.61 (s, 1H), 8.95 (d, J = 3.9 Hz, 1H), 8.49 (d, J = 8.2 Hz, 1H), 8.10-7.91 (m, 2H), 7.75-7.64 (m, 1H), 7.52 (d, J = 8.5 Hz, 1H), 7.46-7.21 (m, 5H), 6.98 (t, J = 6.4 Hz, 1H), 6.86 (d, J = 7.0 Hz, 1H). |
| 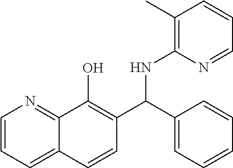 [SMILES: Cc1cccnc1NC(c1ccccc1)c1ccc2cccnc2c1O] | 2 | 7-(((3-methylpyridin-2- yl)amino)(phenyl)methyl)quinolin-8-ol LC-MS: m/z 342 (M + H)⁺. 1H NMR (400 MHz, DMSO-d6) δ (ppm): 10.02 (br s, 1H), 8.84 (d, J = 1.6 Hz, 1H), 8.29 (d, J = 8.2 Hz, 1H), 7.82 (d, J = 4.6 Hz, 1H), 7.72 (d, J = 8.5 Hz, 1H), 7.53 (dd, J = 7.7, 3.7 Hz, 1H), 7.42-7.33 (m, 3H), 7.28-7.25 (m, 3H), 7.19-7.15 (m, 1H), 7.04 (d, J = 8.4 Hz, 1H), 6.47 (t, J = 5.7 Hz, 1H), 6.39 (d, J = 7.6 Hz, 1H), 2.20 (s, 3H). |
| 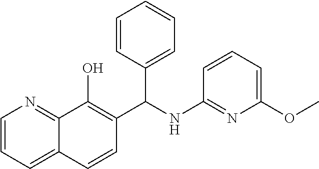 [SMILES: COc1cccc(NC(c2ccccc2)c2ccc3cccnc3c2O)n1] | 2 | 7-(((6-methoxypyridin-2- yl)amino)(phenyl)methyl)quinolin-8-ol LC-MS: m/z 358 (M + H)⁺. 1H NMR (400 MHz, DMSO-d6) δ (ppm): 9.91 (s, 1H), 8.84 (dd, J = 4.2, 1.6 Hz, 1H), 8.33-8.21 (m, 1H), 7.64 (d, J = 8.5 Hz, 1H), 7.53 (dd, J = 8.3, 4.2 Hz, 1H), 7.44-7.34 (m, 4H), 7.33-7.24 (m, 3H), 7.20 (t, J = 7.3 Hz, 1H), 6.80 (d, J = 8.1 Hz, 1H), 6.21 (d, J = 7.9 Hz, 1H), 5.84 (d, J = 7.7 Hz, 1H), 3.61 (s, 3H). |
| 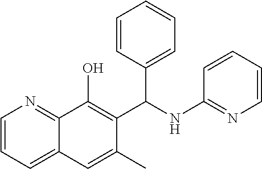 [SMILES: Cc1cc2cccnc2c(O)c1C(Nc1ccccn1)c1ccccc1] | 2 | 6-methyl-7-(phenyl(pyridin-2- ylamino)methyl)quinolin-8-ol LC-MS: m/z 342 (M + H)⁺. 1H NMR (400 MHz, DMSO-d6) δ 9.84 (s, 1H), 8.70 (dd, J = 4.1, 1.6 Hz, 1H), 8.15 (dd, J = 8.3, 1.6 Hz, 1H), 7.89 (dd, J = 5.0, 1.8 Hz, 1H), 7.44 (dd, J = 8.3, 4.2 Hz, 1H), 7.33-7.29 (m, 1H), 7.19-7.18 (m, 5H), 7.13-7.08 (m, 1H), 6.94 (s, 2H), 6.71 (d, J = 8.4 Hz, 1H), 6.42 (dd, J = 6.9, 5.1 Hz, 1H), 2.38 (s, 3H) |
| 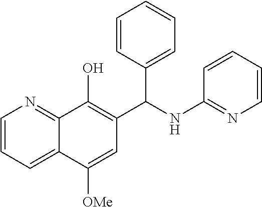 [SMILES: COc1cc(C(Nc2ccccn2)c2ccccc2)c(O)c2ncccc12] | 2 | 5-methoxy-7-(phenyl(pyridin-2- ylamino)methyl)quinolin-8-ol LC-MS: m/z 358 (M + H)⁺. 1H NMR (400 MHz, DMSO-d6) δ 9.37 (brs, 1H), 8.87 (dd, J = 4.2, 1.7 Hz, 1H), 8.44 (dd, J = 8.4, 1.7 Hz, 1H), 7.94 (dd, J = 5.1, 1.9 Hz, 1H), 7.52 (dd, J = 8.5, 4.2 Hz, 1H), 7.47-7.33 (m, 4H), 7.31-7.27 (m, 2H), 7.23-7.12 (m, 2H), 6.91 (d, J = 9.0 Hz, 1H), 6.68 (d, J = 8.4 Hz, 1H), 6.52-6.44 (m, 1H), 3.87 (s, 3H). |
| 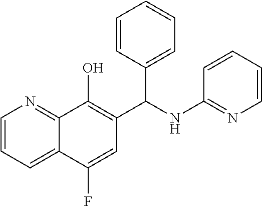 [SMILES: Oc1c(C(Nc2ccccn2)c2ccccc2)cc(F)c2cccnc12] | 2 | 5-fluoro-7-(phenyl(pyridin-2- ylamino)methyl)quinolin-8-ol LC-MS: m/z 346 (M + H)⁺. 1H NMR (400 MHz, DMSO-d6) δ 9.97 (brs, 1H), 8.94 (dd, J = 4.2, 1.6 Hz, 1H), 8.40 (dd, J = 8.4, 1.7 Hz, 1H), 7.95-7.91 (m, 1H), 7.64 (dd, J = 8.5, 4.2 Hz, 1H), 7.53-7.25 (m, 7H), 7.25-7.15 (m, 1H), 6.88 (d, J = 8.6 Hz, 1H), 6.68 (d, J = 8.4 Hz, 1H), 6.49 (dd, J = 6.9, 5.1 Hz, 1H). |
| 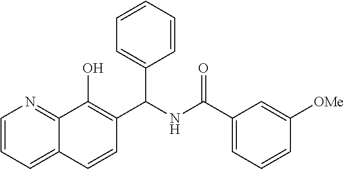 [SMILES: COc1cccc(C(=O)NC(c2ccccc2)c2ccc3cccnc3c2O)c1] | 4 | N-((8-hydroxyquinolin-7-yl)(phenyl)methyl)-3- methoxybenzamide LC-MS: m/z 385 (M + H)⁺. 1H NMR (400 MHz, DMSO-d6) δ (ppm): 10.01 (br s, 1H), 9.22 (d, J = 8.7 Hz, 1H), 8.87 (dd, J = 4.2, 1.6 Hz, 1H), 8.32 (dd, J = 8.3, 1.6 Hz, 1H), 7.69 (d, J = 8.6 Hz, 1H), 7.61-7.50 (m, 2H), 7.48- 7.36 (m, 3H), 7.34-7.28 (m, 4H), 7.27-7.20 (m, 1H), 7.11 (dd, J = 7.9, 2.2 Hz, 1H), 6.99 (d, J = 8.7 Hz, 1H), 3.80 (s, 3H). |
| 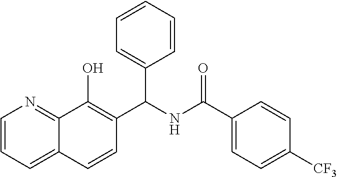 [SMILES: O=C(NC(c1ccccc1)c1ccc2cccnc2c1O)c1ccc(C(F)(F)F)cc1] | 4 | N-((8-hydroxyquinolin-7-yl)(phenyl)methyl)-4- (trifluoromethyl)benzamide LC-MS: m/z 423 (M + H)⁺. 1H NMR (400 MHz, DMSO-d6) δ (ppm): 10.08 (br s, 1H), 9.48 (d, J = 8.6 Hz, 1H), 8.87 (dd, J = 4.2, 1.6 Hz, 1H), 8.32 (dd, J = 8.4, 1.6 Hz, 1H), 8.12 (d, J = 8.1 Hz, 2H), 7.86 (d, J = 8.3 Hz, 2H), 7.66 (d, J = 8.6 Hz, 1H), 7.57 (dd, J = 8.2, 4.2 Hz, 1H), 7.44 (d, J = 8.6 Hz, 1H), 7.34-7.33 (m, 4H), 7.29-7.20 (m, 1H), 7.00 (d, J = 8.6 Hz, 1H). |
| 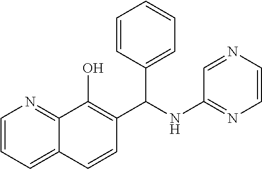 [SMILES: Oc1c(C(Nc2cnccn2)c2ccccc2)ccc2cccnc12] | 3 | 7-(phenyl(pyrazin-2-ylamino)methyl)quinolin-8-ol LC-MS: m/z 329 (M + H)⁺. 1H NMR (400 MHz, DMSO) δ 10.01 (br s, 1H), 8.90-8.82 (m, 1H), 8.34-8.25 (m, 1H), 8.14 (d, J = 1.4 Hz, 1H), 7.95 (d, J = 8.1 Hz, 1H), 7.88 (dd, J = 2.8, 1.4 Hz, 1H), 7.66 (d, J = 2.8 Hz, 1H), 7.59- 7.50 (m, 2H), 7.45-7.38 (m, 1H), 7.37-7.27 (m, 4H), 7.25-7.16 (m, 1H), 6.80 (d, J = 8.1 Hz, 1H). |
| 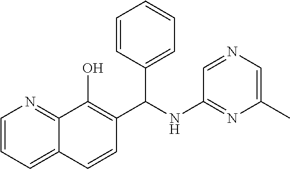 [SMILES: Cc1cncc(NC(c2ccccc2)c2ccc3cccnc3c2O)n1] | 3 | 7-(((6-methylpyrazin-2- yl)amino)(phenyl)methyl)quinolin-8-ol LC-MS: m/z 343 (M + H)⁺. 1H NMR (400 MHz, CD3OD) δ(ppm): 8.80 (dd, J = 4.2, 1.5 Hz, 1H), 8.21 (dd, J = 8.3, 1.5 Hz, 1H), 7.78 (s, 1H), 7.54 (s, 1H), 7.51-7.43 (m, 2H), 7.40 (d, J = 7.3 Hz, 2H), 7.35 (d, J = 8.6 Hz, 1H), 7.32- 7.25 (m, 2H), 7.25-7.18 (m, 1H), 6.79 (s, 1H), 2.27 (s, 3H) |
| 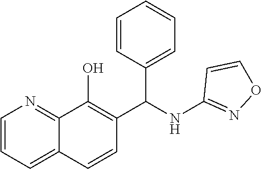 [SMILES: Oc1c(C(Nc2ccon2)c2ccccc2)ccc2cccnc12] | 3 | 7-((isoxazol-3-ylamino)(phenyl)methyl)quinolin-8- ol LC-MS: m/z 318 (M + H)⁺. 1H NMR (400 MHz, DMSO-d6) δ (ppm): 10.00 (br s, 1H), 8.89-8.81 (m, 1H), 8.34 (d, J = 1.6 Hz, 1H), 8.31-8.24 (m, 1H), 7.60 (d, J = 8.5 Hz, 1H), 7.54 (dd, J = 8.3, 4.2 Hz, 1H), 7.44-7.34 (m, 3H), 7.30 (t, J = 7.6 Hz, 2H), 7.24-7.13 (m, 2H), 6.28 (d, J = 8.2 Hz, 1H), 6.03 (d, J = 1.6 Hz, 1H). |
| 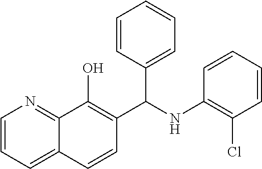 [SMILES: Oc1c(C(Nc2ccccc2Cl)c2ccccc2)ccc2cccnc12] | 1 | 7-(((2- chlorophenyl)amino)(phenyl)methyl)quinolin-8-ol LC-MS: m/z 361 (M + H)⁺. 1H NMR (400 MHz, DMSO-d6) δ (ppm): 10.23 (br s, 1H), 8.92-8.83 (m, 1H), 8.34-8.24 (m, 1H), 7.65 (d, J = 8.5 Hz, 1H), 7.59-7.53 (m, 1H), 7.47- 7.40 (m, 3H), 7.37-7.20 (m, 4H), 7.11-6.95 (m, 1H), 6.71-6.64 (m, 1H), 6.61 (td, J = 7.7, 1.4 Hz, 1H), 6.21 (d, J = 7.3 Hz, 1H), 5.76 (d, J = 7.3 Hz, 1H). |
| 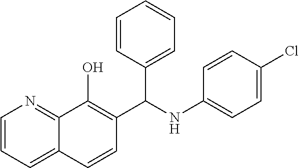 [SMILES: Oc1c(C(Nc2ccc(Cl)cc2)c2ccccc2)ccc2cccnc12] | 1 | 7-(((4- chlorophenyl)amino)(phenyl)methyl)quinolin-8-ol LC-MS: m/z 361 (M + H)⁺. 1H NMR (400 MHz, DMSO-d6) δ (ppm): 10.05 (br s, 1H), 8.89-8.81 (m, 1H), 8.31-8.24 (m, 1H), 7.61-7.50 (m, 2H), 7.41-7.37 (m, 3H), 7.32 (t, J = 7.5 Hz, 2H), 7.23-7.21 (m, 1H), 7.03 (d, J = 8.8 Hz, 2H), 6.70 (d, J = 7.1 Hz, 1H), 6.63 (d, J = 8.8 Hz, 2H), 6.11 (d, J = 7.0 Hz, 1H). |
| 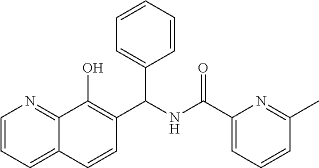 [SMILES: Cc1cccc(C(=O)NC(c2ccccc2)c2ccc3cccnc3c2O)n1] | 4 | N-((8-hydroxyquinolin-7-yl)(phenyl)methyl)-6- methylpicolinamide LC-MS: m/z 370 (M + H)⁺. 1H NMR (400 MHz, DMSO-d6) δ (ppm): 10.20 (br s, 1H), 9.60 (d, J = 8.7 Hz, 1H), 8.87 (d, J = 3.7 Hz, 1H), 8.34 (d, J = 8.3 Hz, 1H), 7.93-7.84 (m, 2H), 7.68 (d, J = 8.4 Hz, 1H), 7.57 (dd, J = 8.1, 4.0 Hz, 1H), 7.53-7.42 (m, 2H), 7.38-7.28 (m, 4H), 7.23 (t, J = 6.9 Hz, 1H), 6.72 (d, J = 9.1 Hz, 1H), 2.58 (s, 3H). |
| 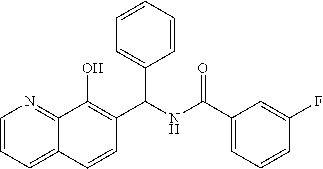 [SMILES: O=C(NC(c1ccccc1)c1ccc2cccnc2c1O)c1cccc(F)c1] | 4 | 3-fluoro-N-((8-hydroxyquinolin-7- yl)(phenyl)methyl)benzamide LC-MS: m/z 373 (M + H)⁺. 1H NMR (400 MHz, DMSO-d6) δ (ppm): 10.04 (s, 1H), 9.32 (d, J = 8.5 Hz, 1H), 8.87 (dd, J = 4.1, 1.5 Hz, 1H), 8.32 (dd, J = 8.3, 1.4 Hz, 1H), 7.82- 7.72 (m, 2H), 7.66 (d, J = 8.6 Hz, 1H), 7.59-7.49 (m, 2H), 7.46-7.37 (m, 2H), 7.36-7.29 (m, 4H), 7.28-7.21 (m, 1H), 6.98 (d, J = 8.5 Hz, 1H). |
| 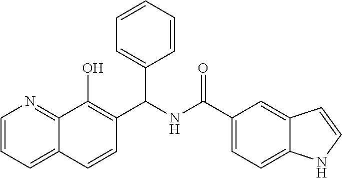 [SMILES: O=C(NC(c1ccccc1)c1ccc2cccnc2c1O)c1ccc2[nH]ccc2c1] | 4 | N-((8-hydroxyquinolin-7-yl)(phenyl)methyl)-1H- indole-5-carboxamide LC-MS: m/z 394 (M + H)⁺. 1H NMR (400 MHz, DMSO-d6) δ (ppm): 11.31 (s, 1H), 9.99 (br s, 1H), 9.05 (d, J = 8.8 Hz, 1H), 8.87 (dd, J = 4.2, 1.5 Hz, 1H), 8.32 (dd, J = 8.3, 1.5 Hz, 1H), 8.25 (s, 1H), 7.77 (d, J = 8.6 Hz, 1H), 7.70 (dd, J = 8.6, 1.6 Hz, 1H), 7.56 (dd, J = 8.3, 4.2 Hz, 1H), 7.47-7.40 (m, 3H), 7.37-7.27 (m, 4H), 7.24 (d, J = 6.8 Hz, 1H), 7.03 (d, J = 8.9 Hz, 1H), 6.54 (s, 1H). |
| 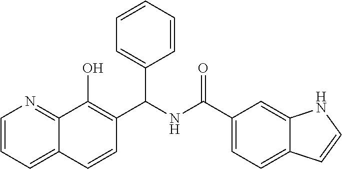 [SMILES: O=C(NC(c1ccccc1)c1ccc2cccnc2c1O)c1ccc2cc[nH]c2c1] | 4 | N-((8-hydroxyquinolin-7-yl)(phenyl)methyl)-1H- indole-6-carboxamide LC-MS: m/z 394 (M + H)⁺. 1H NMR (400 MHz, DMSO-d6) δ (ppm): 11.36 (s, 1H), 10.02 (br s, 1H), 9.12 (d, J = 9.1 Hz, 1H), 8.92-8.83 (m, 1H), 8.32 (d, J = 6.8 Hz, 1H), 8.03 (s, 1H), 7.77 (d, J = 8.6 Hz, 1H), 7.64-7.53 (m, 3H), 7.52-7.48 (m, 1H), 7.44 (d, J = 8.6 Hz, 1H), 7.37-7.27 (m, 4H), 7.24 (d, J = 6.8 Hz, 1H), 7.04 (d, J = 8.7 Hz, 1H), 6.49 (s, 1H). |
| 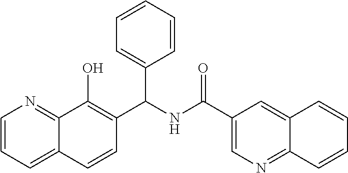 [SMILES: O=C(NC(c1ccccc1)c1ccc2cccnc2c1O)c1cnc2ccccc2c1] | 4 | N-((8-hydroxyquinolin-7- yl)(phenyl)methyl)quinoline-3-carboxamide LC-MS: m/z 406 (M + H)⁺. 1H NMR (400 MHz, DMSO-d6) δ (ppm): 10.03 (br s, 1H), 9.58 (t, J = 13.3 Hz, 1H), 9.32 (d, J = 2.1 Hz, 1H), 8.95 (d, J = 1.8 Hz, 1H), 8.91-8.84 (m, 1H), 8.33 (d, J = 8.3 Hz, 1H), 8.15-8.06 (m, 2H), 7.87 (t, J = 7.7 Hz, 1H), 7.74-7.66 (m, 2H), 7.57 (dd, J = 8.3, 4.2 Hz, 1H), 7.46 (d, J = 8.6 Hz, 1H), 7.42-7.31 (m, 4H), 7.26 (t, J = 6.7 Hz, 1H), 7.05 (d, J = 8.4 Hz, 1H). |
| 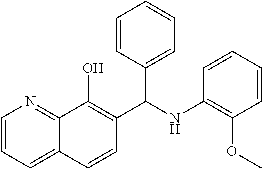 [SMILES: COc1ccccc1NC(c1ccccc1)c1ccc2cccnc2c1O] | 1 | 7-(((2- methoxyphenyl)amino)(phenyl)methyl)quinolin-8- ol LC-MS: m/z 357 (M + H)⁺. 1H NMR (400 MHz, DMSO-d6) δ (ppm): 10.05 (br s, 1H), 8.85 (dd, J = 4.1, 1.5 Hz, 1H), 8.29 (dd, J = 8.3, 1.5 Hz, 1H), 7.60 (d, J = 8.5 Hz, 1H), 7.54 (dd, J = 8.3, 4.2 Hz, 1H), 7.45-7.37 (m, 3H), 7.32 (t, J = 7.5 Hz, 2H), 7.23 (t, J = 7.3 Hz, 1H), 6.84 (dd, J = 7.9, 1.1 Hz, 1H), 6.64 (dd, J = 7.6, 6.5 Hz, 1H), 6.58-6.49 (m, 1H), 6.47-6.38 (m, 1H), 6.11 (d, |
| | | J = 6.5 Hz, 1H), 5.27 (d, J = 6.6 Hz, 1H), 3.81 (s, |
| | | 3H). |
| 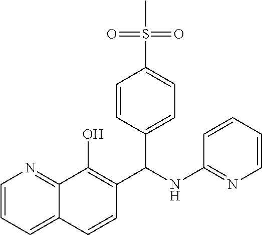 [SMILES: CS(=O)(=O)c1ccc(C(Nc2ccccn2)c2ccc3cccnc3c2O)cc1] | 2 | 7-((4-(methylsulfonyl)phenyl)(pyridin-2- ylamino)methyl)quinolin-8-ol LC-MS: m/z 406 (M + H)⁺. 1H NMR (400 MHz, DMSO-d6) δ (ppm): 9.31 (s, 1H), 8.97-8.87 (m, 1H), 8.37 (dd, J = 8.4, 1.5 Hz, 1H), 8.03 (d, J = 5.8 Hz, 1H), 7.97-7.90 (m, 3H), 7.75-7.65 (m, 2H), 7.66-7.53 (m, 2H), 7.51-7.36 (m, 2H), 7.16 (d, J = 8.9 Hz, 1H), 6.95-6.88 (m, 1H), 6.82 (t, J = 7.5 Hz, 1H), 3.16 (s, 3H). |
| 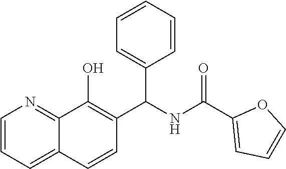 [SMILES: O=C(NC(c1ccccc1)c1ccc2cccnc2c1O)c1ccco1] | 4 | N-((8-hydroxyquinolin-7-yl)(phenyl)methyl)furan- 2-carboxamide LC-MS: m/z 345 (M + H)⁺. 1H NMR (400 MHz, DMSO-d6) δ (ppm): 10.02 (br s, 1H), 9.10 (d, J = 8.9 Hz, 1H), 8.87 (dd, J = 4.1, 1.4 Hz, 1H), 8.32 (dd, J = 8.3, 1.3 Hz, 1H), 7.86 (d, J = 0.9 Hz, 1H), 7.67 (d, J = 8.5 Hz, 1H), 7.56 (dd, J = 8.3, 4.2 Hz, 1H), 7.43 (d, J = 8.6 Hz, 1H), 7.36-7.20 (m, 6H), 6.90 (d, J = 8.9 Hz, 1H), 6.64 (dd, J = 3.4, 1.7 Hz, 1H). |
| 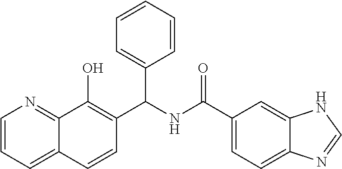 [SMILES: O=C(NC(c1ccccc1)c1ccc2cccnc2c1O)c1ccc2nc[nH]c2c1] | 4 | N-((8-hydroxyquinolin-7-yl)(phenyl)methyl)-1H- benzo[d]imidazole-6-carboxamide LC-MS: m/z 395 (M + H)⁺. 1H NMR (400 MHz, DMSO-d6) δ (ppm): 12.65 (s, 1H), 10.04 (br s, 1H), 9.21 (d, J = 8.6 Hz, 1H), 8.87 (dd, J = 4.1, 1.5 Hz, 1H), 8.37-8.29 (m, 2H), 8.16 (s, 1H), 7.82 (d, J = 7.9 Hz, 1H), 7.76 (d, J = 8.5 Hz, 1H), 7.59-7.54 (m, 1H), 7.44 (d, J = 8.6 Hz, 1H), 7.39-7.29 (m, 4H), 7.24 (t, J = 6.7 Hz, 1H), 7.04 (d, J = 8.7 Hz, 1H). |
| 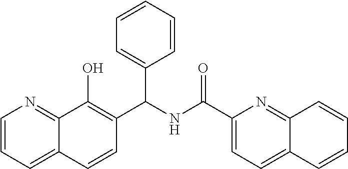 [SMILES: O=C(NC(c1ccccc1)c1ccc2cccnc2c1O)c1ccc2ccccc2n1] | 4 | N-((8-hydroxyquinolin-7- yl)(phenyl)methyl)quinoline-2-carboxamide LC-MS: m/z 406 (M + H)⁺. 1H NMR (400 MHz, DMSO-d6) δ (ppm): 9.92 (d, J = 9.1 Hz, 1H), 8.89 (dd, J = 4.2, 1.5 Hz, 1H), 8.60 (d, J = 8.5 Hz, 1H), 8.35 (dd, J = 8.4, 1.5 Hz, 1H), 8.20 (dd, J = 10.9, 8.6 Hz, 2H), 8.10 (d, J = 8.1 Hz, 1H), 7.88 (dd, J = 11.2, 4.2 Hz, 1H), 7.79-7.69 (m, 2H), 7.58 (dd, J = 8.3, 4.2 Hz, 1H), 7.47 (d, J = 8.5 Hz, 1H), 7.41 (d, J = 7.5 Hz, 2H), 7.33 (t, J = 7.6 Hz, 2H), 7.25 (t, J = 7.3 Hz, 1H), 6.82 (d, J = 9.1 |
| | | Hz, 1H). |
| 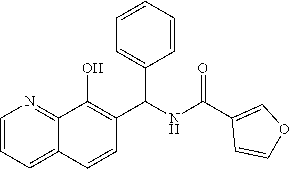 [SMILES: O=C(NC(c1ccccc1)c1ccc2cccnc2c1O)c1ccoc1] | 4 | N-((8-hydroxyquinolin-7-yl)(phenyl)methyl)furan- 3-carboxamide LC-MS: m/z 345 (M + H)⁺. 1H NMR (400 MHz, DMSO-d6) δ (ppm): 9.98 (br s, 1H), 8.92 (d, J = 8.6 Hz, 1H), 8.87 (d, J = 2.8 Hz, 1H), 8.38-8.28 (m, 2H), 7.74 (s, 1H), 7.62 (d, J = 8.5 Hz, 1H), 7.57 (dd, J = 8.2, 4.2 Hz, 1H), 7.45 (d, J = 8.5 Hz, 1H), 7.37-7.21 (m, 5H), 7.01-6.87 (m, 2H). |
| 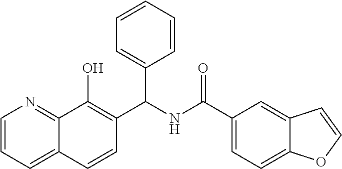 [SMILES: O=C(NC(c1ccccc1)c1ccc2cccnc2c1O)c1ccc2occc2c1] | 4 | N-((8-hydroxyquinolin-7- yl)(phenyl)methyl)benzofuran-5-carboxamide LC-MS: m/z 395 (M + H)⁺. 1H NMR (400 MHz, DMSO-d6) δ (ppm): 10.03 (br s, 1H), 9.26 (d, J = 8.7 Hz, 1H), 8.88 (dd, J = 4.2, 1.5 Hz, 1H), 8.36-8.28 (m, 2H), 8.09 (d, J = 2.2 Hz, 1H), 7.92 (dd, J = 8.7, 1.7 Hz, 1H), 7.73 (d, J = 8.6 Hz, 1H), 7.68 (d, J = 8.7 Hz, 1H), 7.57 (dd, J = 8.3, 4.2 Hz, 1H), 7.45 (d, J = 8.6 Hz, 1H), 7.37- 7.30 (m, 4H), 7.27-7.21 (m, 1H), 7.07 (dd, J = 5.9, 5.2 Hz, 1H), 7.03 (d, J = 8.7 Hz, 1H). |
| 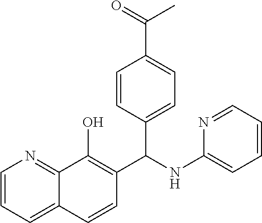 [SMILES: CC(=O)c1ccc(C(Nc2ccccn2)c2ccc3cccnc3c2O)cc1] | 2 | 1-(4-((8-hydroxyquinolin-7-yl)(pyridin-2- ylamino)methyl)phenyl)ethan-1-one LC-MS: m/z 370 (M + H)⁺. 1H NMR (400 MHz, DMSO-d6) δ (ppm): 10.03 (brs, 1H), 8.86 (d, J = 3.2 Hz, 1H), 8.30 (dd, J = 8.4, 1.2 Hz, 1H), 7.93-7.88 (m, 3H), 7.59-7.53 (m, 4H), 7.51-7.38 (m, 3H), 6.93 (d, J = 8.4 Hz, 1H), 6.71 (d, J = 8.4 Hz, 1H), 6.51-6.48 (m, 1H), 2.53 (s, 3H) |
| 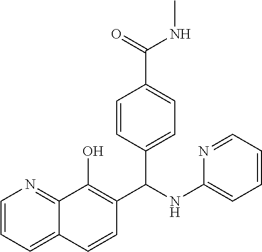 [SMILES: CNC(=O)c1ccc(C(Nc2ccccn2)c2ccc3cccnc3c2O)cc1] | 2 | 4-((8-hydroxyquinolin-7-yl)(pyridin-2- ylamino)methyl)-N-methylbenzamide LC-MS: m/z 385 (M + H)⁺. 1H NMR (400 MHz, DMSO-d6) δ (ppm): 9.88 (br s, 1H), 8.85 (dd, J = 4.2, 1.5 Hz, 1H), 8.38-8.26 (m, 2H), 7.91 (d, J = 3.6 Hz, 1H), 7.73 (d, J = 8.3 Hz, 2H), 7.59 (d, J = 8.5 Hz, 1H), 7.54 (dd, J = 8.3, 4.2 Hz, 1H), 7.45-7.30 (m, 5H), 6.90 (d, J = 8.3 Hz, 1H), 6.69 (d, J = 8.4 Hz, 1H), 6.52-6.43 (m, 1H), 2.74 (d, J = 4.5 Hz, 3H). |
| 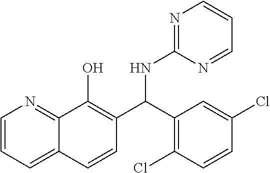 [SMILES: Oc1c(C(Nc2ncccn2)c2cc(Cl)ccc2Cl)ccc2cccnc12] | 3 | 7-((2,5-dichlorophenyl)(pyrimidin-2- ylamino)methyl)quinolin-8-ol LC-MS: m/z 397 (M + H)⁺. 1H NMR (400 MHz, DMSO-d6) δ (ppm): 10.03 (br s, 1H), 8.86 (dd, J = 4.1, 1.4 Hz, 1H), 8.31 (dd, J = 10.1, 3.1 Hz, 3H), 7.99 (d, J = 8.4 Hz, 1H), 7.56 (dd, J = 8.3, 4.2 Hz, 1H), 7.47 (t, J = 7.0 Hz, 1H), 7.43-7.30 (m, 4H), 7.06 (d, J = 8.4 Hz, 1H), 6.62 (t, J = 4.8 Hz, 1H). |
| 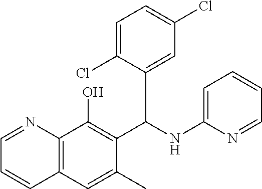 [SMILES: Cc1cc2cccnc2c(O)c1C(Nc1ccccn1)c1cc(Cl)ccc1Cl] | 2 | 7-((2,5-dichlorophenyl)(pyridin-2- ylamino)methyl)-6-methylquinolin-8-ol LC-MS: m/z 410 (M + H)⁺. 1H NMR (400 MHz, DMSO-d6) δ 8.74 (dd, J = 4.2, 1.6 Hz, 1H), 8.21 (dd, J = 8.4, 1.6 Hz, 1H), 7.94 (d, J = 5.2 Hz, 1H), 7.62 (d, J = 2.5 Hz, 1H), 7.51 (dd, J = 8.2, 4.2 Hz, 1H), 7.45-7.20 (m, 5H), 6.83 (d, J = 7.5 Hz, 1H), 6.78 (d, J = 8.4 Hz, 1H), 6.52-6.49 (m, 1H), 2.46 (s, 3H). |
| 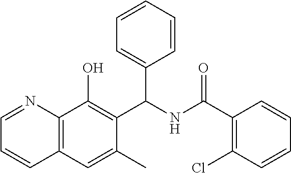 [SMILES: Cc1cc2cccnc2c(O)c1C(NC(=O)c1ccccc1Cl)c1ccccc1] | 4 | 2-chloro-N-((8-hydroxy-6-methylquinolin-7- yl)(phenyl)methyl)benzamide LC-MS: m/z 403 (M + H)⁺. 1H NMR (400 MHz, DMSO-d6) δ 8.94 (d, J = 8.4 Hz, 1H), 8.80 (dd, J = 4.2, 1.6 Hz, 1H), 8.25 (dd, J = 8.4, 1.6 Hz, 1H), 7.58-7.36 (m, 5H), 7.31-7.29 (m, 5H), 7.26-7.19 (m, 1H), 6.96 (d, J = 8.3 Hz, 1H), 2.48 (s, 3H). |
| 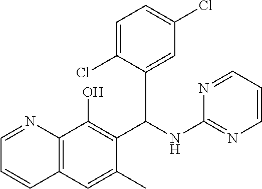 [SMILES: Cc1cc2cccnc2c(O)c1C(Nc1ncccn1)c1cc(Cl)ccc1Cl] | 3 | 7-((2,5-dichlorophenyl)(pyrimidin-2- ylamino)methyl)-6-methylquinolin-8-ol LC-MS: m/z 411 (M + H)⁺. 1H NMR (400 MHz, DMSO-d6) δ 8.76 (d, J = 4.2 Hz, 1H), 8.33 (d, J = 4.8 Hz, 2H), 8.22 (d, J = 7.9 Hz, 1H), 7.60 (d, J = 8.3 Hz, 1H), 7.53-7.51 (m, 2H), 7.42 (d, J = 8.4 Hz, 1H), 7.37-7.32 (m, 1H), 7.26 (s, 1H), 6.88 (d, J = 8.4 Hz, 1H), 6.66 (dd, J = 4.8, 4.8 Hz, 1H), 2.45 (s, 3H). |
| 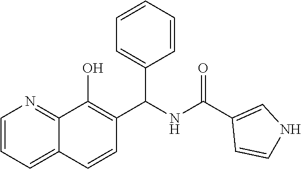 [SMILES: O=C(NC(c1ccccc1)c1ccc2cccnc2c1O)c1cc[nH]c1] | 4 | N-((8-hydroxyquinolin-7-yl)(phenyl)methyl)-1H- pyrrole-3-carboxamide LC-MS: m/z 344 (M + H)⁺. 1H NMR (400 MHz, DMSO-d6) δ (ppm): 11.17 (s, 1H), 9.93 (br s, 1H), 8.87 (d, J = 2.9 Hz, 1H), 8.46 (d, J = 9.0 Hz, 1H), 8.32 (d, J = 8.0 Hz, 1H), 7.71 (d, J = 8.5 Hz, 1H), 7.56 (dd, J = 8.2, 4.1 Hz, 1H), 7.49-7.39 (m, 2H), 7.34-7.18 (m, 5H), 6.95 (d, J = 9.0 Hz, 1H), 6.76 (d, J = 2.1 Hz, 1H), 6.60 (s, 1H). |
| 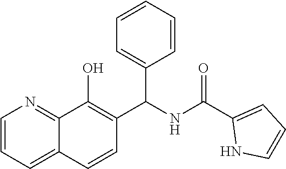 [SMILES: O=C(NC(c1ccccc1)c1ccc2cccnc2c1O)c1ccc[nH]1] | 4 | N-((8-hydroxyquinolin-7-yl)(phenyl)methyl)-1H- pyrrole-2-carboxamide LC-MS: m/z 344 (M + H)⁺. 1H NMR (400 MHz, DMSO-d6) δ (ppm): 11.47 (s, 1H), 9.98 (br s, 1H), 8.86 (dd, J = 4.1, 1.3 Hz, 1H), 8.68 (d, J = 8.9 Hz, 1H), 8.32 (dd, J = 8.3, 1.3 Hz, 1H), 7.67 (d, J = 8.6 Hz, 1H), 7.56 (dd, J = 8.3, 4.2 Hz, 1H), 7.44 (d, J = 8.6 Hz, 1H), 7.34-7.20 (m, 5H), 6.99 (s, 1H), 6.95 (d, J = 8.9 Hz, 1H), 6.88 (s, 1H), 6.10 (d, J = 3.3 Hz, 1H). |
| 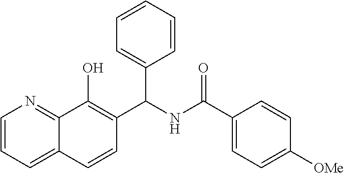 [SMILES: COc1ccc(C(=O)NC(c2ccccc2)c2ccc3cccnc3c2O)cc1] | 4 | N-((8-hydroxyquinolin-7-yl)(phenyl)methyl)-4- methoxybenzamide LC-MS: m/z 385 (M + H)⁺. 1H NMR (400 MHz, DMSO-d6) δ (ppm): 9.99 (br s, 1H), 9.06 (d, J = 9.0 Hz, 1H), 8.87 (d, J = 2.9 Hz, 1H), 8.32 (d, J = 7.2 Hz, 1H), 7.93 (d, J = 8.8 Hz, 2H), 7.70 (d, J = 8.5 Hz, 1H), 7.56 (dd, J = 8.3, 4.2 Hz, 1H), 7.43 (d, J = 8.6 Hz, 1H), 7.33-7.27 (m, 4H), 7.26-7.19 (m, 1H), 7.03-6.96 (m, 3H), 3.81 (s, 3H). |
| 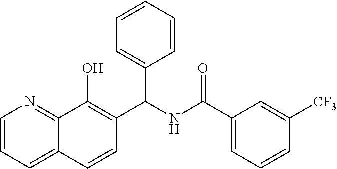 [SMILES: O=C(NC(c1ccccc1)c1ccc2cccnc2c1O)c1cccc(C(F)(F)F)c1] | 4 | N-((8-hydroxyquinolin-7-yl)(phenyl)methyl)-3- (trifluoromethyl)benzamide LC-MS: m/z 423 (M + H)⁺. 1H NMR (400 MHz, DMSO-d6) δ (ppm): 10.05 (br s, 1H), 9.51 (d, J = 8.4 Hz, 1H), 8.90-8.84 (m, 1H), 8.36-8.28 (m, 2H), 8.25 (d, J = 7.8 Hz, 1H), 7.92 (d, J = 7.8 Hz, 1H), 7.73 (t, J = 7.8 Hz, 1H), 7.63 (d, J = 8.6 Hz, 1H), 7.57 (dd, J = 8.3, 4.2 Hz, 1H), 7.44 (d, J = 8.6 Hz, 1H), 7.36-7.29 (m, 4H), 7.28-7.20 (m, 1H), 7.00 (d, J = 8.4 Hz, 1H). |
| 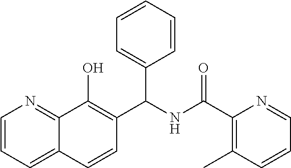 [SMILES: Cc1cccnc1C(=O)NC(c1ccccc1)c1ccc2cccnc2c1O] | 4 | N-((8-hydroxyquinolin-7-yl)(phenyl)methyl)-3- methylpicolinamide LC-MS: m/z 370 (M + H)⁺. 1H NMR (400 MHz, DMSO-d6) δ (ppm): 10.20 (br s, 1H), 9.50 (d, J = 9.1 Hz, 1H), 8.87 (dd, J = 4.1, 1.4 Hz, 1H), 8.48 (d, J = 4.1 Hz, 1H), 8.34 (dd, J = 8.3, 1.3 Hz, 1H), 7.77 (d, J = 7.7 Hz, 1H), 7.69 (d, J = 8.5 Hz, 1H), 7.57 (dd, J = 8.3, 4.2 Hz, 1H), 7.51-7.42 (m, 2H), 7.37 (d, J = 7.4 Hz, 2H), 7.32 (t, J = 7.6 Hz, 2H), 7.24 (t, J = 7.1 Hz, 1H), 6.81 (d, J = 9.1 Hz, 1H), 2.52 (s, 3H). |
| 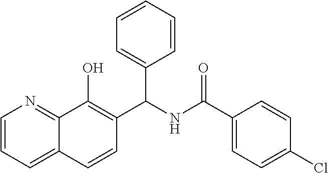 [SMILES: O=C(NC(c1ccccc1)c1ccc2cccnc2c1O)c1ccc(Cl)cc1] | 4 | 4-chloro-N-((8-hydroxyquinolin-7- yl)(phenyl)methyl)benzamide LC-MS: m/z 389 (M + H)⁺. 1H NMR (400 MHz, DMSO-d6) δ (ppm): 10.05 (br s, 1H), 9.31 (d, J = 8.5 Hz, 1H), 8.87 (d, J = 4.0 Hz, 1H), 8.32 (d, J = 8.2 Hz, 1H), 7.96 (d, J = 8.5 Hz, 2H), 7.66 (d, J = 8.6 Hz, 1H), 7.59-7.51 (m, 3H), 7.43 (d, J = 8.6 Hz, 1H), 7.37-7.29 (m, 4H), 7.28-7.18 (m, 1H), 6.98 (d, J = 8.6 Hz, 1H). |
| 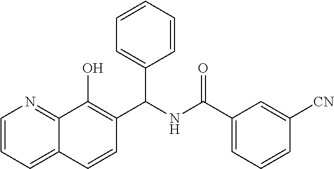 [SMILES: N#Cc1cccc(C(=O)NC(c2ccccc2)c2ccc3cccnc3c2O)c1] | 4 | 3-cyano-N-((8-hydroxyquinolin-7- yl)(phenyl)methyl)benzamide LC-MS: m/z 380 (M + H)⁺. 1H NMR (400 MHz, DMSO-d6) δ (ppm): 10.04 (br s, 1H), 9.43 (d, J = 8.4 Hz, 1H), 8.87 (dd, J = 4.1, 1.5 Hz, 1H), 8.42 (s, 1H), 8.32 (dd, J = 8.3, 1.4 Hz, 1H), 8.22 (d, J = 8.1 Hz, 1H), 8.02 (d, J = 7.7 Hz, 1H), 7.70 (t, J = 7.8 Hz, 1H), 7.63 (d, J = 8.6 Hz, 1H), 7.57 (dd, J = 8.3, 4.2 Hz, 1H), 7.44 (d, J = 8.7 Hz, 1H), 7.34-7.30 (m, 4H), 7.29-7.20 (m, 1H), 6.97 (d, J = 8.3 Hz, 1H). |
| 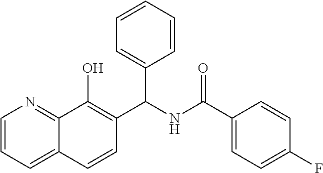 [SMILES: O=C(NC(c1ccccc1)c1ccc2cccnc2c1O)c1ccc(F)cc1] | 4 | 4-fluoro-N-((8-hydroxyquinolin-7- yl)(phenyl)methyl)benzamide LC-MS: m/z 373 (M + H)⁺. 1H NMR (400 MHz, DMSO-d6) δ (ppm): 10.01 (br s, 1H), 9.25 (d, J = 8.6 Hz, 1H), 8.90-8.84 (m, 1H), 8.32 (d, J = 8.4 Hz, 1H), 8.06-7.96 (m, 2H), 7.67 (d, J = 8.6 Hz, 1H), 7.56 (dd, J = 8.3, 4.2 Hz, 1H), 7.43 (d, J = 8.6 Hz, 1H), 7.35-7.27 (m, 6H), 7.26-7.18 (m, 1H), 6.98 (d, J = 8.6 Hz, 1H). |
| 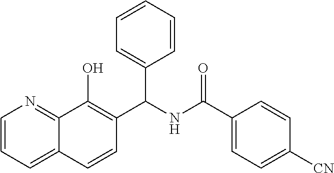 [SMILES: N#Cc1ccc(C(=O)NC(c2ccccc2)c2ccc3cccnc3c2O)cc1] | 4 | 4-cyano-N-((8-hydroxyquinolin-7- yl)(phenyl)methyl)benzamide LC-MS: m/z 380 (M + H)⁺. 1H NMR (400 MHz, DMSO-d6) δ (ppm): 10.05 (br s, 1H), 9.50 (d, J = 8.5 Hz, 1H), 8.87 (dd, J = 4.1, 1.4 Hz, 1H), 8.32 (dd, J = 8.3, 1.3 Hz, 1H), 8.08 (d, J = 8.4 Hz, 2H), 7.97 (d, J = 8.4 Hz, 2H), 7.64 (d, J = 8.6 Hz, 1H), 7.57 (dd, J = 8.3, 4.2 Hz, 1H), 7.43 (d, J = 8.6 Hz, 1H), 7.37-7.30 (m, 4H), 7.28-7.21 (m, 1H), 6.98 (d, J = 8.5 Hz, 1H). |
| 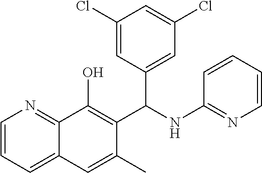 [SMILES: Cc1cc2cccnc2c(O)c1C(Nc1ccccn1)c1cc(Cl)cc(Cl)c1] | 2 | 7-((3,5-dichlorophenyl)(pyridin-2- ylamino)methyl)-6-methylquinolin-8-ol LC-MS: m/z 410 (M + H)⁺. 1H NMR (400 MHz, DMSO-d6) δ (ppm): 9.95 (br s, 1H), 8.78 (d, J = 3.5 Hz, 1H), 8.23 (d, J = 8.1 Hz, 1H), 7.99 (d, J = 4.4 Hz, 1H), 7.53 (dd, J = 8.2, 4.1 Hz, 1H), 7.45-7.39 (m, 2H), 7.29 (s, 1H), 7.23-7.17 (m, 2H), 7.13 (d, J = 8.0 Hz, 1H), 7.00 (d, J = 7.8 Hz, 1H), 6.84 (d, J = 8.4 Hz, 1H), 6.60-6.49 (m, 1H), 2.54 (s, 3H). |
| 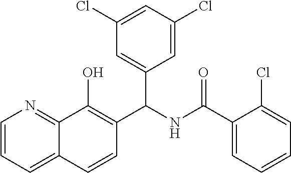 [SMILES: O=C(NC(c1cc(Cl)cc(Cl)c1)c1ccc2cccnc2c1O)c1ccccc1Cl] | 4 | 2-chloro-N-((3,5-dichlorophenyl)(8- hydroxyquinolin-7-yl)methyl)benzamide LC-MS: m/z 457 (M + H)⁺. 1H NMR (400 MHz, DMSO-d6) δ (ppm): 10.03- 9.85 (m, 1H), 8.55 (s, 1H), 8.35 (d, J = 7.8 Hz, 1H), 7.68-7.59 (m, 3H), 7.54-7.41 (m, 6H), 7.35 (t, J = 7.5 Hz, 1H), 6.94 (d, J = 8.4 Hz, 1H), 6.72 (t, J = 8.0 Hz, 1H). |
| 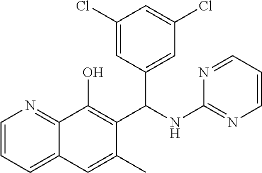 [SMILES: Cc1cc2cccnc2c(O)c1C(Nc1ncccn1)c1cc(Cl)cc(Cl)c1] | 3 | 7-((3,5-dichlorophenyl)(pyrimidin-2- ylamino)methyl)-6-methylquinolin-8-ol LC-MS: m/z 411 (M + H)⁺. 1H NMR (400 MHz, CDCl3) δ (ppm): 8.62 (dd, J = 4.2, 1.4 Hz, 1H), 8.23 (d, J = 4.8 Hz, 2H), 7.99 (dd, J = 8.3, 1.4 Hz, 1H), 7.34 (dd, J = 8.3, 4.2 Hz, 1H), 7.20 (s, 1H), 7.14 (dd, J = 4.2, 2.3 Hz, 2H), 6.91 (s, 2H), 6.49 (t, J = 4.8 Hz, 1H), 2.63 (s, 3H). |
| 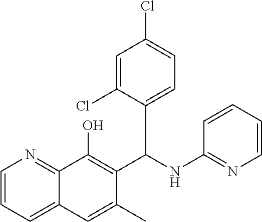 [SMILES: Cc1cc2cccnc2c(O)c1C(Nc1ccccn1)c1ccc(Cl)cc1Cl] | 2 | 7-((2,4-dichlorophenyl)(pyridin-2- ylamino)methyl)-6-methylquinolin-8-ol LC-MS: m/z 410 (M + H)⁺. 1H NMR (400 MHz, DMSO) δ 9.67 (br s, 1H), 8.80 (d, J = 3.6 Hz, 1H), 8.27 (d, J = 7.9 Hz, 1H), 7.98 (d, J = 4.5 Hz, 1H), 7.66 (d, J = 8.5 Hz, 1H), 7.57 (dd, J = 8.2, 4.2 Hz, 1H), 7.53 (d, J = 1.9 Hz, 1H), 7.44 (t, J = 7.9 Hz, 2H), 7.30 (s, 1H), 7.25 (d, J = 7.4 Hz, 1H), 6.89 (d, J = 7.4 Hz, 1H), 6.85-6.76 (m, 1H), 6.59-6.48 (m, 1H), 2.51 (s, 3H). |
| 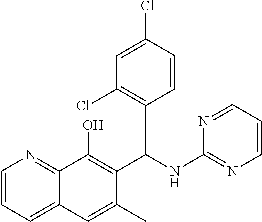 [SMILES: Cc1cc2cccnc2c(O)c1C(Nc1ncccn1)c1ccc(Cl)cc1Cl] | 3 | 7-((2,4-dichlorophenyl)(pyrimidin-2- ylamino)methyl)-6-methylquinolin-8-ol LC-MS: m/z 411 (M + H)⁺. 1H NMR (400 MHz, DMSO-d6) δ (ppm): 9.88 (br s, 1H), 8.77 (d, J = 3.4 Hz, 1H), 8.31 (d, J = 4.8 Hz, 2H), 8.22 (d, J = 7.9 Hz, 1H), 7.56-7.44 (m, 4H), 7.40-7.33 (m, 1H), 7.23 (s, 1H), 6.90 (d, J = 8.3 Hz, 1H), 6.64 (t, J = 4.8 Hz, 1H), 2.45 (s, 3H). |
| 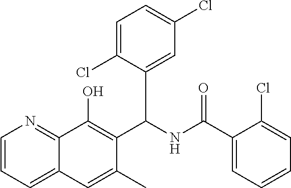 [SMILES: Cc1cc2cccnc2c(O)c1C(NC(=O)c1ccccc1Cl)c1cc(Cl)ccc1Cl] | 4 | 2-chloro-N-((2,5-dichlorophenyl)(8-hydroxy-6- methylquinolin-7-yl)methyl)benzamide LC-MS: m/z 471, 473 (M + H)⁺. 1H NMR (400 MHz, CD3OD) δ (ppm): 8.73 (d, J = 7.2 Hz, 1H), 8.17 (d, J = 8.4 Hz, 1H), 7.66 (s, 1H), 7.51-7.35 (m, 6H) 7.30-7.26 (m, 2H), 6.95 (s, 1H), 2.62 (s, 3H) |
| 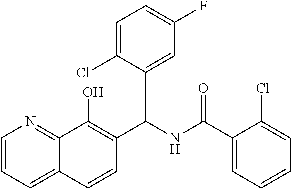 [SMILES: O=C(NC(c1cc(F)ccc1Cl)c1ccc2cccnc2c1O)c1ccccc1Cl] | 4 | 2-chloro-N-((2-chloro-5-fluorophenyl)(8- hydroxyquinolin-7-yl)methyl)benzamide LC-MS: m/z 441, 443 (M + H)⁺. 1H NMR (400 MHz, CDCl3) δ (ppm): 8.72 (d, J = 4.0 Hz, 1H), 8.34 (d, J = 8.0 Hz, 1H), 7.72-7.65 (m, 3H), 7.53 (dd, J = 8.0 Hz, 4.4 Hz, 1H) 7.40- 7.20 (m, 6H), 6.93 (d, J = 8.0 Hz, 1H), 6.88 (dd, J = 8.0 Hz, 2.4 Hz, 1H) |
| 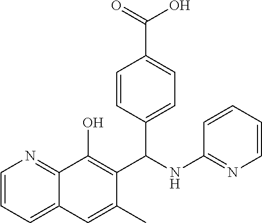 [SMILES: Cc1cc2cccnc2c(O)c1C(Nc1ccccn1)c1ccc(C(=O)O)cc1] | 2 | 4-((8-hydroxy-6-methylquinolin-7-yl)(pyridin-2- ylamino)methyl)benzoic acid LC-MS: m/z 386 (M + H)⁺. 1H NMR (400 MHz, DMSO-d6) δ (ppm): 12.84 (br s, 1H), 9.99 (br s, 1H), 8.85 (d, J = 4.0 Hz, 1H), 8.30 (d, J = 8.0 Hz, 1H), 8.04 (d, J = 4.4 Hz, 1H), 7.92 (d, J = 8.0 Hz, 2H) 7.60 (dd, J = 8.4 Hz, 4.4 Hz, 1H), 7.47 (t, J = 7.6 Hz, 1H), 7.43 (d, J = 8.0 Hz, 2H), 7.34 (s, 1H), 7.17-7.11 (m, 2H), 6.88 (d, J = 8.4 Hz, 1H), 6.58 (t, J = 6.0 Hz, 1H), 2.58 (s, 3H) |
| 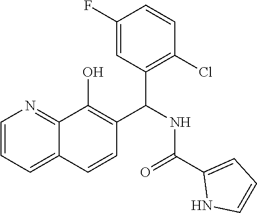 [SMILES: O=C(NC(c1cc(F)ccc1Cl)c1ccc2cccnc2c1O)c1ccc[nH]1] | 4 | N-((2-chloro-5-fluorophenyl)(8-hydroxyquinolin- 7-yl)methyl)-1H-pyrrole-2-carboxamide LC-MS: m/z 396 (M + H)⁺. 1H NMR (400 MHz, CD3OD) δ (ppm): 8.66 (s, 1H), 8.27 (d, J = 7.6 Hz, 1H), 7.53-7.51 (m, 1H), 7.43-7.37 (m, 3H), 7.10-7.07 (m, 1H), 7.04-6.83 (m, 4H), 6.12 (s, 1H) |
| 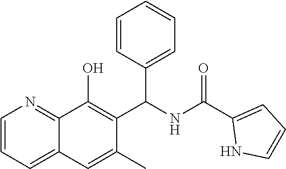 [SMILES: Cc1cc2cccnc2c(O)c1C(NC(=O)c1ccc[nH]1)c1ccccc1] | 4 | N-((8-hydroxy-6-methylquinolin-7- yl)(phenyl)methyl)-1H-pyrrole-2-carboxamide LC-MS: m/z 358 (M + H)⁺. 1H NMR (400 MHz, DMSO-d6) δ (ppm): 11.63 (br s, 1H), 8.80 (dd, J = 4.4 Hz, 1.6 Hz, 1H), 8.40 (dd, J = 8.4 Hz, 3.6 Hz, 1H), 8.25 (dd, J = 8.4 Hz, 1.6 Hz, 1H), 7.54 (dd, J = 8.4 Hz, 4.0 Hz, 1H), 7.32-7.24 (m, 3H), 7.23-7.20 (m, 3H), 6.90 (dd, J = 4.0 Hz, 1.2 Hz, 1H), 6.85 (d, J = 8.8 Hz, 1H), 6.75 (s, 1H), 6.12 (dd, J = 3.6 Hz, 2.0 Hz, 1H), 2.54 (s, 3H) |
| 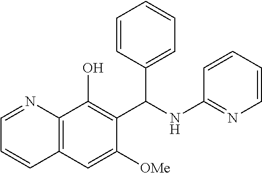 [SMILES: COc1cc2cccnc2c(O)c1C(Nc1ccccn1)c1ccccc1] | 2 | 6-methoxy-7-(phenyl(pyridin-2- ylamino)methyl)quinolin-8-ol LC-MS: m/z 358 (M + H)⁺. 1H NMR (400 MHz, DMSO-d6) δ (ppm): 10.02 (br s, 1H), 8.70 (dd, J = 4.0 Hz, 1.2 Hz, 1H), 8.21 (dd, J = 8.4 Hz, 1.2 Hz, 1H), 7.97 (dd, J = 4.8 Hz, 1.2 Hz, 1H), 7.51 (dd, J = 8.4 Hz, 4.4 Hz, 1H), 7.42-7.33 (m, 3H), 7.25-7.13 (m, 4H), 6.93-6.88 (m, 2H), 6.75 (d, J = 8.4 Hz, 1H), 6.51 (dd, J = 6.4 Hz, 1.2 Hz, 1H), 3.89 (s, 3H) |
| 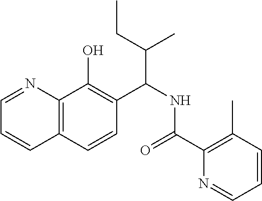 [SMILES: CCC(C)C(NC(=O)c1ncccc1C)c1ccc2cccnc2c1O] | 5 | N-(1-(8-hydroxyquinolin-7-yl)-2-methylbutyl)-3- methylpicolinamide LC-MS: m/z 350 (M + H)⁺. 1H NMR (400 MHz, CD3OD) δ (ppm): mixture of stereoisomers (dd, J = 4.4 Hz, 1.2 Hz, 1H), 8.43 (d, J = 8.4 Hz, 1.2 Hz, 1H), 8.23 (dd, J = 8.4 Hz, 0.8 Hz, 1H), 7.72-7.66 (m, 1H), 7.50-7.45 (m, 2H), 7.41-7.33 (m, 2H), 5.40 (d, J = 8.0 Hz, 0.5H), 5.29 (d, J = 7.6 Hz, 0.5H), 2.56 and 2.55 (two set of s, total 3H), 2.27-2.23 (m, 1H), 1.81-1.72 (m, 0.5H), 1.48-1.30 (m, 1H), 1.21-1.16 (m, 0.5H), 1.07 (d, J = 6.8 Hz, 1.5H), 0.98 (t, J = 7.2 Hz, 1.5H), 0.92 (t, J = 7.2 Hz, 1.5H), 0.84 (d, J = 6.8 Hz, 1.5H) |
| 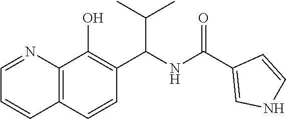 [SMILES: CC(C)C(NC(=O)c1cc[nH]c1)c1ccc2cccnc2c1O] | 5 | N-(1-(8-hydroxyquinolin-7-yl)-2-methylpropyl)- 1H-pyrrole-3-carboxamide LC-MS: m/z 310 (M + H)⁺. 1H NMR (400 MHz, DMSO-d6) δ (ppm): 11.12 (br s, 1H), 9.75 (br s, 1H), 8.85 (dd, J = 4.0 Hz, 1.2 Hz, 1H), 8.29 (dd, J = 8.4 Hz, 1.6 Hz, 1H), 7.87 (d, J = 9.2 Hz, 1H), 7.67 (d, J = 8.4 Hz, 1H), 7.53 (dd, J = 8.4 Hz, 4.4 Hz, 1H), 7.41-7.34 (m, 2H), |
| | | 6.74 (dd, J = 4.4 Hz, 2.4 Hz, 1H), 6.51 (dd, J = 4.0 |
| | | Hz, 2.4 Hz, 1H), 5.36 (t, J = 9.2 Hz, 1H), 2.22- |
| | | 2.13 (m, 1H), 1.02 (d, J = 6.8 Hz, 3H), 0.78 (d, J = |
| | | 6.8 Hz, 3H) |
| 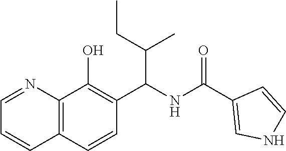 [SMILES: CCC(C)C(NC(=O)c1cc[nH]c1)c1ccc2cccnc2c1O] | 5 | N-(1-(8-hydroxyquinolin-7-yl)-2-methylbutyl)-1H- pyrrole-3-carboxamide LC-MS: m/z 324 (M + H)⁺. 1H NMR (400 MHz, CD3OD) δ (ppm): mixture of stereoisomers. 8.79 (dd, J = 4.0 Hz, 1.6 Hz, 1H), 8.23 (d, J = 8.4 Hz, 1.6 Hz, 1H), 7.52 (d, J = 8.4 Hz, 1H), 7.47 (dd, J = 8.4 Hz, 4.0 Hz, 1H), 7.39- 7.33 (m, 2H), 6.74 (dd, J = 4.8 Hz, 2.4 Hz, 1H), 6.56 (dt, J = 4.8 Hz, 2.0 Hz, 1H), 5.39-5.28 (m, 1H), 2.23-2.16 (m, 1H), 1.82-1.76 (m, 0.5H), 1.38- |
| | | 1.29 (m, 1H), 1.16-1.09 (m, 0.5H), 1.08 (d, J = 6.8 |
| | | Hz, 1.5H), 0.98 (t, J = 7.2 Hz, 1.5H), 0.88 (t, J = |
| | | 7.2 Hz, 1.5H), 0.80 (d, J = 6.8 Hz, 1.5H) |
| 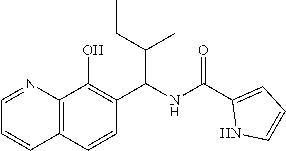 [SMILES: CCC(C)C(NC(=O)c1ccc[nH]1)c1ccc2cccnc2c1O] | 5 | N-(1-(8-hydroxyquinolin-7-yl)-2-methylbutyl)-1H- pyrrole-2-carboxamide LC-MS: m/z 324 (M + H)⁺. 1H NMR (400 MHz, CD3OD) δ (ppm): mixture of stereoisomers. 8.79 (dd, J = 4.0 Hz, 1.2 Hz, 1H), 8.22 (d, J = 8.4 Hz, 1.2 Hz, 1H), 7.52 (d, J = 8.4 Hz, 1H), 7.48 (dd, J = 8.4 Hz, 4.4 Hz, 1H), 7.37 (d, J = 8.4 Hz, 1H), 6.89-6.83 (m, 2H), 6.16 (dd, J = 6.0 Hz, 2.4 Hz, 1H), 5.42-5.30 (m, 1H), 2.20-2.16 (m, 1H), 1.83-1.75 (m, 0.5H), 1.40-1.31 (m, 1H), |
| | | 1.18-1.08 (m, 0.5H), 1.05 (d, J = 6.8 Hz, 1.5H), |
| | | 0.98 (t, J = 7.2 Hz, 1.5H), 0.92 (t, J = 7.2 Hz, |
| | | 1.5H), 0.83 (d, J = 6.8 Hz, 1.5H) |
| 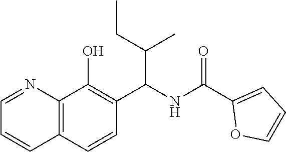 [SMILES: CCC(C)C(NC(=O)c1ccco1)c1ccc2cccnc2c1O] | 5 | N-(1-(8-hydroxyquinolin-7-yl)-2- methylbutyl)furan-2-carboxamide LC-MS: m/z 325 (M + H)⁺. 1H NMR (400 MHz, DMSO-d6) δ (ppm): mixture of stereoisomers. 9.81 (br s, 1H), 8.85 (dd, J = 4.0 Hz, 1.6 Hz, 1H), 8.51 (d, J = 9.2 Hz, 0.5H), 8.43 (d, J = 9.2 Hz, 0.5H), 8.29 (dd, J = 8.0 Hz, 1.2 Hz, 1H), 7.82 (s, 1H), 7.68 (dd, J = 8.4 Hz, 0.8 Hz, 1H), 7.53 (dd, J = 8.4 Hz, 4.0 Hz, 1H), 7.40 (d, J = 8.4 Hz, 1H), 7.13 (dd, J = 11.6 Hz, 3.6 Hz, 1H), |
| | | 6.63-6.59 (m, 1H), 5.47 (t, J = 9.2 Hz, 0.5H), 5.41 |
| | | (t, J = 9.2 Hz, 0.5H), 2.08-2.00 (m, 1H), 1.73-1.58 |
| | | (m, 0.5H), 1.33-1.17 (m, 1H), 1.12-1.02 (m, 0.5H), |
| | | 0.98 (d, J = 6.8 Hz, 1.5H), 0.90 (t, J = 7.2 Hz, |
| | | 1.5H), 0.81 (t, J = 7.2 Hz, 1.5H), 0.71 (d, J = 6.8 |
| | | Hz, 1.5H) |
| 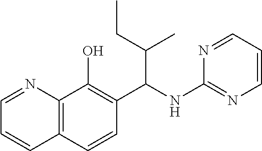 [SMILES: CCC(C)C(Nc1ncccn1)c1ccc2cccnc2c1O] | 3 | 7-(2-methyl-1-(pyrimidin-2- ylamino)butyl)quinolin-8-ol LC-MS: m/z 309 (M + H)⁺. 1H NMR (400 MHz, CD3OD) δ (ppm): mixture of stereoisomers. 8.79 (d, J = 3.2 Hz 1H), 8.29-8.17 (m, 3H), 7.49 (d, J = 8.4 Hz, 1H), 7.45 (dd, J = 8.0 Hz, 4.0 Hz, 1H), 7.33 (d, J = 8.4 Hz, 1H), 6.54 (dd, J = 8.4 Hz, 4.0 Hz, 1H), 5.40 (d, J = 8.0 Hz, 0.67H), 5.27 (d, J = 7.6 Hz, 0.33H), 2.24-2.18 (m, 1H), 1.83-1.77 (m, 0.33H), 1.43-1.10 (m, 1.67H), |
| | | 1.04 (d, J = 6.8 Hz, 2H), 0.95 (t, J = 7.2 Hz, 1H), |
| | | 0.90 (t, J = 7.2 Hz, 2H), 0.82 (d, J = 6.8 Hz, 1H) |
| 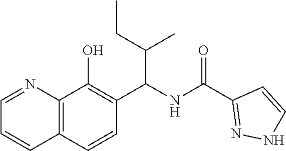 [SMILES: CCC(C)C(NC(=O)c1cc[nH]n1)c1ccc2cccnc2c1O] | 5 | N-(1-(8-hydroxyquinolin-7-yl)-3-methylbutyl)-1H- pyrazole-3-carboxamide LC-MS: m/z 325 (M + H)⁺. 1H NMR (400 MHz, DMSO-d6) δ (ppm): mixture of stereoisomers. 13.24 (br s, 1H), 9.70 (br s, 1H), 8.85 (dd, J = 4.4 Hz, 1.6 Hz 1H), 8.38-8.30 (m, 2H), 7.80 (br s, 1H), 7.63-7.56 (m, 1H), 7.53 (dd, J = 8.4 Hz, 4.0 Hz, 1H), 7.39 (d, J = 8.4 Hz, 1H), 6.61 (br s, 1H), 5.37-5.30 (m, 1H), 2.10-2.02 (m, 1H), 1.69-1.63 (m, 0.5H), 1.41-1.26 (m, 1H), 1.22- |
| | | 1.02 (m, 0.5H), 0.95 (d, J = 6.8 Hz, 1.5H), 0.91 (d, |
| | | J = 6.8 Hz, 1.5H), 0.76 (d, J = 6.8 Hz, 1.5H), 0.68 |
| | | (d, J = 6.8 Hz, 1.5H) |
| 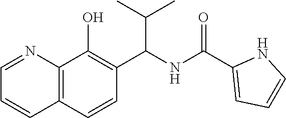 [SMILES: CC(C)C(NC(=O)c1ccc[nH]1)c1ccc2cccnc2c1O] | 5 | N-(1-(8-hydroxyquinolin-7-yl)-2-methylpropyl)- 1H-pyrrole-2-carboxamide LC-MS: m/z 310 (M + H)⁺. 1H NMR (400 MHz, DMSO-d6) δ (ppm): 11.42 (br s, 1H), 9.72 (br s, 1H), 8.85 (dd, J = 4.4 Hz, 1.6 Hz 1H), 8.30 (dd, J = 8.4 H, 1.6 Hz, 1H), 8.07 (d, J = 9.6 Hz, 1H), 7.66 (d, J = 8.8 Hz, 1H), 7.53 (dd, J = 8.4 Hz, 4.4 Hz, 1H), 7.40 (d, J = 8.8 Hz, 1H), |
| | | 6.90 (dd, J = 2.4 Hz, 1.6 Hz, 1H), 6.84 (dd, J = 3.6 |
| | | Hz, 2.4 Hz, 1H), 6.09 (dd, J = 3.6 Hz, 2.4 Hz, 1H), |
| | | 5.40 (d, J = 9.2 Hz, 1H), 2.24-2.12 (m, 1H), 1.02 |
| | | (d, J = 6.8 Hz, 3H), 0.79 (d, J = 6.8 Hz, 3H) |
| 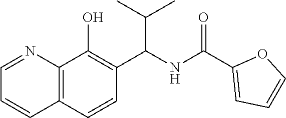 [SMILES: CC(C)C(NC(=O)c1ccco1)c1ccc2cccnc2c1O] | 5 | N-(1-(8-hydroxyquinolin-7-yl)-2- methylpropyl)furan-2-carboxamide LC-MS: m/z 311 (M + H)⁺. 1H NMR (400 MHz, CD3OD) δ (ppm): 8.81 (d, J = 4.4 Hz, 1.6 Hz 1H), 8.23 (dd, J = 8.4 H, 1.6 Hz, 1H), 7.66 (dd, J = 1.6 Hz, 0.8 Hz, 1H), 7.50 (d, J = 8.4 Hz, 1H), 7.47 (dd, J = 8.4 Hz, 4.0 Hz, 1H), 7.37 (d, J = 8.4 Hz, 1H), 7.10 (dd, J = 3.6 Hz, 0.8 Hz, |
| | | 1H), 6.57 (dd, J = 3.6 Hz, 1.6 Hz, 1H), 5.21 (d, J = |
| | | 9.2 Hz, 1H), 2.44-2.36 (m, 1H), 1.12 (d, J = 6.4 Hz, |
| | | 3H), 0.86 (d, J = 6.8 Hz, 3H) |
| 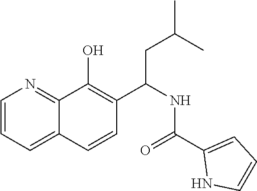 [SMILES: CC(C)CC(NC(=O)c1ccc[nH]1)c1ccc2cccnc2c1O] | 5 | N-(1-(8-hydroxyquinolin-7-yl)-3-methylbutyl)-1H- pyrrole-2-carboxamide LC-MS: m/z 324 (M + H)⁺. 1H NMR (400 MHz, CD3OD) δ (ppm): 8.79 (d, J = 3.2 Hz 1H), 8.21 (d, J = 7.6 Hz, 1H), 7.54 (d, J = 8.8 Hz, 1H), 7.46 (dd, J = 8.0 Hz, 4.0 Hz, 1H), 7.37 (d, J = 8.4 Hz, 1H), 6.90 (t, J = 2.0 Hz, 2H), 6.17 (t, J = 3.2 Hz, 1H), 5.69 (dd, J = 9.2 Hz, 6.0 Hz, 1H), 1.96-1.87 (m, 1H), 1.83-1.76 (m, 1H), 1.74- 1.65 (m, 1H), 1.01 (d, J = 6.4 Hz, 6H) |
| 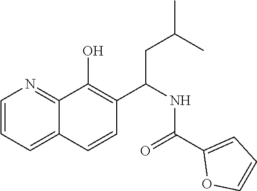 [SMILES: CC(C)CC(NC(=O)c1ccco1)c1ccc2cccnc2c1O] | 5 | N-(1-(8-hydroxyquinolin-7-yl)-3- methylbutyl)furan-2-carboxamide LC-MS: m/z 325 (M + H)⁺. 1H NMR (400 MHz, CD3OD) δ (ppm): mixture of two rotamers, ation is ca. 5:1. 8.82 (major) and 8.53 (minor) (d, J = 3.6 Hz, 1H), 8.32-8.22 (m, 1H), 7.70-7.50 (m, 1H), 7.26 and 7.07 (two set of d, J = 8.4 Hz, total 1H), 7.04 (s, 1H), 6.55 (s, 1H), 5.91 and 5.62 (two set of t, J = 7.2 Hz, total 1H), 2.02-1.94 (m, 1H), 1.90-1.74 (m, 1H), 1.68-1.58 (m, 1H), 1.04 and 1.02 (major, two set of d, J = 6.8 Hz, 5H), 0.62 and 0.39 (minor, two set of d, J = |
| | | 6.4 Hz, 1H) |
| 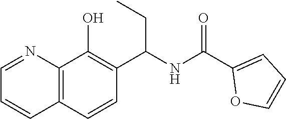 [SMILES: CCC(NC(=O)c1ccco1)c1ccc2cccnc2c1O] | 5 | N-(1-(8-hydroxyquinolin-7-yl)propyl)furan-2- carboxamide LC-MS: m/z 297 (M + H)⁺. 1H NMR (400 MHz, CD3OD) δ (ppm): 8.83 (d, J = 3.2 Hz, 1H), 8.27 (d, J = 7.6 Hz, 1H), 7.58 (s, 1H), 7.49 (dd, J = 8.4 Hz, 3.2 Hz, 2H), 7.25 (d, J = 8.4 Hz, 1H), 7.10 (d, J = 3.6 Hz, 1H), 6.55 (dd, J = 3.2 Hz, 1.6 Hz, 1H), 5.37 (t, J = 7.2 Hz, 1H), 2.09- |
| | | 2.01 (m, 2H), 1.00 (t, J = 7.2 Hz, 3H) |
| 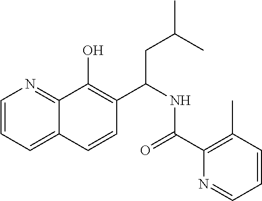 [SMILES: Cc1cccnc1C(=O)NC(CC(C)C)c1ccc2cccnc2c1O] | 5 | N-(1-(8-hydroxyquinolin-7-yl)-3-methylbutyl)-3- methylpicolinamide LC-MS: m/z 350 (M + H)⁺. 1H NMR (400 MHz, CD3OD) δ (ppm): 8.81 (dd, J = 4.0 Hz, 1.2 Hz, 1H), 8.44 (d, J = 4.0 Hz, 1H), 8.22 (dd, J = 8.4 Hz, 1.6 Hz, 1H), 7.70 (d, J = 7.6 Hz, 1H), 7.54 (d, J = 8.4 Hz, 1H), 7.47 (dd, J = 8.4 Hz, 4.4 Hz, 1H), 7.42-7.36 (m, 1H), 5.67 (dd, J = 8.8 Hz, 2.4 Hz, 1H), 2.54 (s, 3H), 1.97-1.83 (m, 2H), 1.71-1.63 (m, 2H), 1.03 (d, J = 6.4 Hz, 3H), 1.02 (d, J = 6.4 Hz, 3H) |
| 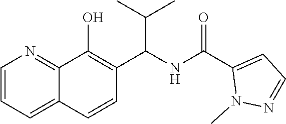 [SMILES: CC(C)C(NC(=O)c1ccnn1C)c1ccc2cccnc2c1O] | 5 | N-(1-(8-hydroxyquinolin-7-yl)-2-methylpropyl)-1- methyl-1H-pyrazole-5-carboxamide LC-MS: m/z 325 (M + H)⁺. 1H NMR (400 MHz, CD3OD) δ (ppm): 9.01 (dd, J = 5.2 Hz, 1.6 Hz 1H), 8.94 (dd, J = 8.4 Hz, 0.8 Hz, 1H), 7.95 (dd, J = 8.4 Hz, 1.2 Hz, 1H), 7.83 (d, J = 8.8 Hz, 1H), 7.78 (d, J = 8.8 Hz, 1H), 7.47 (d, J = 2.0 Hz, 1H), 6.89 (d. J = 2.0 Hz, 1H), 5.18 (d, J = |
| | | 10.4 Hz, 1H), 4.03 (s, 3H), 2.53-2.45 (m, 1H), |
| | | 1.23 (d, J = 6.4 Hz, 3H), 0.84 (d, J = 6.4 Hz, 3H) |
| 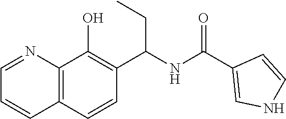 [SMILES: CCC(NC(=O)c1cc[nH]c1)c1ccc2cccnc2c1O] | 5 | N-(1-(8-hydroxyquinolin-7-yl)propyl)-1H-pyrrole- 3-carboxamide LC-MS: m/z 296 (M + H)⁺. 1H NMR (400 MHz, CD3OD) δ (ppm): 8.82 (dd, J = 4.0 Hz, 1.2 Hz, 1H), 8.32 (d, J = 7.6 Hz, 1H), 7.57 (d, J = 8.4 Hz, 1H), 7.53 (dd, J = 8.4 Hz, , 4.4 Hz, 1H), 7.42 (d, J = 8.4 Hz, 1H), 7.38 (dd, J = 4.4 Hz, 2.8 Hz, 1H), 6.75 (dd, J = 4.4 Hz, 1.6 Hz, |
| | | 1H), 6.59 (dd, J = 2.8 Hz, 1.6 Hz, 1H), 5.44 (t, J = |
| | | 7.6 Hz, 1H), 2.03 (penta, J = 7.2 Hz, 2H), 1.02 (t, J = |
| | | 7.2 Hz, 3H) |
| 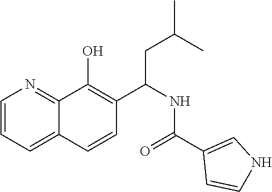 [SMILES: CC(C)CC(NC(=O)c1cc[nH]c1)c1ccc2cccnc2c1O] | 5 | N-(1-(8-hydroxyquinolin-7-yl)-3-methylbutyl)-1H- pyrrole-3-carboxamide LC-MS: m/z 324 (M + H)⁺. 1H NMR (400 MHz, CD3OD) δ (ppm): two rotamer, ratio is 1:1, 8.87 (d, J = 3.2 Hz) and 8.58 (d, J = 4.0 Hz) (total 1H), 8.29 and 8.22 (two set of d, J = 8.0 Hz, total 1H), 7.59-7.52 (m, 1H), 7.47- 7.43 (m, 1H), 7.39-7.32 (m, 1H), 7.34 and 7.07 (two set of d, J = 8.4 Hz, total 1H), 6.73-6.57 (m, total 1H), 5.88 and 5.67 (two set of t, J = 8.0 Hz, 1H), 1.99-1.91 (m) and 1.68-1.22 (two set of m, total 1H), 1.84-1.63 (m, 2H), 1.00 (d, J = 6.8 Hz, |
| | | 3H), 0.58 and 0.42 (two set of d, J = 6.8 Hz, 3H) |
| 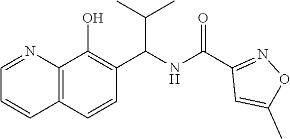 [SMILES: Cc1cc(C(=O)NC(c2ccc3cccnc3c2O)C(C)C)no1] | 5 | N-(1-(8-hydroxyquinolin-7-yl)-2-methylpropyl)-5- methylisoxazole-3-carboxamide LC-MS: m/z 326 (M + H)⁺. 1H NMR (400 MHz, DMSO-d6) δ (ppm): 9.80 (br s, 1H), 8.92 (d, J = 10.0 Hz, 1H), 8.86 (d, J = 2.8 Hz, 1H), 8.30 (d, J = 7.6 Hz, 1H), 7.68 (d, J = 8.8 Hz, 1H), 7.55 (dd, J = 8.0 Hz, 4.0 Hz, 1H), 7.40 (d, J = 8.8 Hz, 1H), 6.51 (s, 1H), 5.34 (d, J = 9.6 Hz, 1H), 2.45 (s, 3H), 2.27-2.22 (m, 1H), 1.04 (d, J = |
| | | 6.4 Hz, 3H), 0.76 (d, J = 6.4 Hz, 3H) |
| 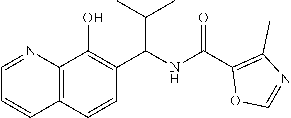 [SMILES: Cc1ncoc1C(=O)NC(c1ccc2cccnc2c1O)C(C)C] | 5 | N-(1-(8-hydroxyquinolin-7-yl)-2-methylpropyl)-4- methyloxazole-5-carboxamide LC-MS: m/z 326 (M + H)⁺. 1H NMR (400 MHz, CD3OD) δ (ppm): 9.01 (dd, J = 4.4 Hz, 1.2 Hz, 1H), 8.98 (dd, J = 8.4 Hz, 1.2 Hz, 1H), 8.21 (s, 1H), 7.97 (dd, J = 8.4 Hz, 4.4 Hz, 1H), 7.87 (d, J = 8.4 Hz, 1H), 7.79 (d, J = 8.8 Hz, 1H), 5.16 (d, J = 10.4 Hz, 1H), 2.58-2.51 (m, 1H), |
| | | 2.41 (s, 3H), 1.22 (d, J = 6.4 Hz, 3H), 0.84 (d, J = |
| | | 6.8 Hz, 3H) |
| 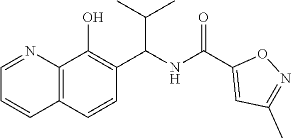 [SMILES: Cc1cc(C(=O)NC(c2ccc3cccnc3c2O)C(C)C)on1] | 5 | N-(1-(8-hydroxyquinolin-7-yl)-2-methylpropyl)-3- methylisoxazole-5-carboxamide LC-MS: m/z 326 (M + H)⁺. 1H NMR (400 MHz, CDCl3) δ (ppm): 8.72 (d, J = 3.6 Hz, 1H), 8.07 (d, J = 8.4 Hz, 1H), 7.97 (d, J = 9.2 Hz, 1H), 7.36 (dd, J = 8.4 Hz, , 4.4 Hz, 1H), 7.32 (d, J = 8.4 Hz, 1H), 7.24 (d, J = 8.4 Hz, 1H), 6.63 (s, 1H), 5.02 (t, J = 9.2 Hz, 1H), 2.41-2.35 (m, 1H), 2.10 (s, 3H), 1.04 (d, J = 6.4 Hz, 3H), 0.81 (d, |
| | | J = 6.8 Hz, 3H) |
| 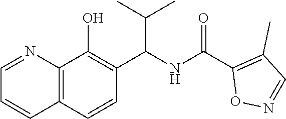 [SMILES: Cc1cnoc1C(=O)NC(c1ccc2cccnc2c1O)C(C)C] | 5 | N-(1-(8-hydroxyquinolin-7-yl)-2-methylpropyl)-4- methylisoxazole-5-carboxamide LC-MS: m/z 326 (M + H)⁺. 1H NMR (400 MHz, CDCl3) δ (ppm): 8.71 (d, J = 3.2 Hz, 1H), 8.09-8.06 (m, 2H), 7.92 (dd, J = 8.4 Hz, 1.2 Hz, 1H), 7.36 (dd, J = 8.4 Hz, , 4.0 Hz, 1H), 7.33 (d, J = 8.4 1H), 7.24 (d, J = 8.4 Hz, 1H), 5.04 (t, J = 9.2 Hz, 1H), 2.43-2.35 (m, 1H), 2.25 (s, |
| | | 3H), 1.03 (d, J = 6.8 Hz, 3H), 0.82 (d, J = 6.8 Hz, |
| | | 3H) |
| 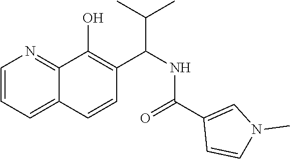 [SMILES: CC(C)C(NC(=O)c1ccn(C)c1)c1ccc2cccnc2c1O] | 5 | N-(1-(8-hydroxyquinolin-7-yl)-2-methylpropyl)-1- methyl-1H-pyrrole-3-carboxamide LC-MS: m/z 324 (M + H)⁺. 1H NMR (400 MHz, CD3OD) δ (ppm): 8.79 (d, J = 3.6 Hz, 1H), 8.22 (d, J = 8.4 Hz, 1H), 7.52-7.44 (m, 2H), 7.35 (d, J = 8.4 Hz, 1H), 7.24 (s, 1H), 6.64 (s, 1H), 6.51 (s, 1H), 5.19 (d, J = 9.6 Hz, 1H), 3.66 (s, 3H), 2.40-2.34 (m, 1H), 1.11 (d, J = 6.4 Hz, 3H), 0.84 (d, J = 6.8 Hz, 3H) |
| 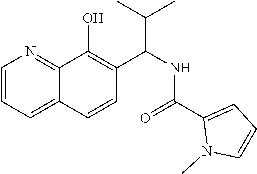 [SMILES: CC(C)C(NC(=O)c1cccn1C)c1ccc2cccnc2c1O] | 5 | N-(1-(8-hydroxyquinolin-7-yl)-2-methylpropyl)-1- methyl-1H-pyrrole-2-carboxamide LC-MS: m/z 324 (M + H)⁺. 1H NMR (400 MHz, CD3OD) δ (ppm): 8.99 (d, J = 4.4 Hz, 1.6 Hz, 1H), 8.96 (d, 8.4 Hz, 1H), 7.95 (dd, J = 8.0 Hz, 4.0 Hz, 1H), 7.85 (d, J = 8.8 Hz, 1H), 7.78 (d, J = 8.8 Hz, 1H), 6.68 (dd, J = 4.0 Hz, 2.4 Hz, 1H), 6.84 (d, J = 2.0 Hz, 1H), 6.06 (dd, J = 4.0 Hz, 1.2 Hz, 1H), 5.04 (d J = 10.4 Hz, 1H), 3.83 (s, 3H), 2.63-2.52 (m, 1H), 1.20 (d, J = 6.8 Hz, 3H), 0.82 (d, J = 6.8 Hz, 3H) |
| 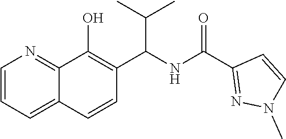 [SMILES: CC(C)C(NC(=O)c1ccn(C)n1)c1ccc2cccnc2c1O] | 5 | N-(1-(8-hydroxyquinolin-7-yl)-2-methylpropyl)-1- methyl-1H-pyrazole-3-carboxamide LC-MS: m/z 325 (M + H)⁺. 1H NMR (400 MHz, CD3OD) δ (ppm): 8.81 (dd, J = 4.4 Hz, 1.6 Hz, 1H), 8.25 (dd, J = 8.4 Hz, 1.6 Hz, 1H), 7.60 (d, J = 2.4 Hz, 1H), 7.51-7.46 (m, 2H), 7.38 (d, J = 8.8 Hz, 1H), 6.68 (d, J = 2.0 Hz, 1H), 5.17 (d J = 8.8 Hz, 1H), 3.95 (s, 3H), 2.47-2.40 (m, 1H), 1.10 (d, J = 6.8 Hz, 3H), 0.87 (d, J = 6.8 Hz, |
| | | 3H) |
| 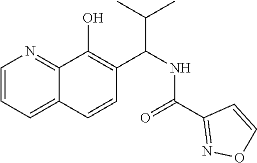 [SMILES: CC(C)C(NC(=O)c1ccon1)c1ccc2cccnc2c1O] | 5 | N-(1-(8-hydroxyquinolin-7-yl)-2- methylpropyl)isoxazole-3-carboxamide LC-MS: m/z 312 (M + H)⁺. 1H NMR (400 MHz, CDCl3) δ (ppm): mixture of rotamer (ratio is ca. 2:1) 8.83-8.72 (m, 2H), 8.39- 8.36 (m, 1H), 7.95-7.89 (m, 1H), 7.76-7.62 (m, 2H), 7.55 (d, J = 8.4 Hz, 1H), 6.71 (minor) and 6.69 (major) (two set of d, J = 1.6 Hz, 1H), 5.13- 5.02 (two set of t, J = 9.2 Hz, 1H), 2.62-2.45 (m, 1H), 1.02 (d, J = 6.8 Hz, 3H), 0.81 (minor) and |
| | | 0.78 (major) (two set of d, J = 6.8 Hz, 3H) |
| 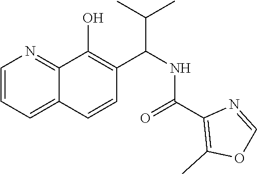 [SMILES: Cc1ocnc1C(=O)NC(c1ccc2cccnc2c1O)C(C)C] | 5 | N-(1-(8-hydroxyquinolin-7-yl)-2-methylpropyl)-5- methyloxazole-4-carboxamide LC-MS: m/z 326 (M + H)⁺. 1H NMR (400 MHz, DMSO-d6) δ (ppm): 9.89 (br s, 1H), 8.86 (dd, J = 4.0 Hz, 1.2 Hz, 1H), 8.40 (d, J = 10.0 Hz, 1H), 8.34 (s, 1H), 8.31 (dd, J = 8.4 Hz, 1.2 Hz, 1H), 7.59 (d, J = 8.8 Hz, 1H), 7.55 (dd, J = 8.4 Hz, 4.0 Hz, 1H), 7.39 (d, J = 8.4 Hz, 1H), 5.17 (t, J = 9.6 Hz, 1H), 2.53 (s, 3H), 2.36-2.28 (m, 1H), 1.01 (d, J = 6.8 Hz, 3H), 0.77 (d, J = 6.4 Hz, 3H) |
| 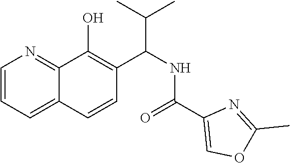 [SMILES: Cc1nc(C(=O)NC(c2ccc3cccnc3c2O)C(C)C)co1] | 5 | N-(1-(8-hydroxyquinolin-7-yl)-2-methylpropyl)-2- methyloxazole-4-carboxamide LC-MS: m/z 326 (M + H)⁺. 1H NMR (400 MHz, CD3OD) δ (ppm): 8.81 (dd, J = 4.0 Hz, 1.2 Hz, 1H), 8.24-8.20 (m, 2H), 7.49- 7.44 (m, 2H), 7.37 (d, J = 8.8 Hz, 1H), 5.15 (d, J = 9.2 Hz, 1H), 2.48 (s, 3H), 2.52-2.40 (m, 1H), 1.09 (d, J = 6.4 Hz, 3H), 0.86 (d, J = 6.4 Hz, 3H) |
| 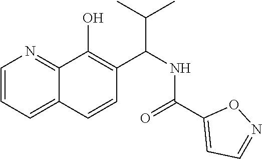 [SMILES: CC(C)C(NC(=O)c1ccno1)c1ccc2cccnc2c1O] | 5 | N-(1-(8-hydroxyquinolin-7-yl)-2- methylpropyl)isoxazole-5-carboxamide LC-MS: m/z 312 (M + H)⁺. 1H NMR (400 MHz, DMSO-d6) δ (ppm): 9.75 (br s, 1H), 9.16 (d, J = 8.8 Hz, 1H), 8.85 (d, J = 2.0 Hz, 1H), 8.73 (d, J = 1.2 Hz, 1H), 8.29 (d, J = 8.4 Hz, 1.2 Hz, 1H), 7.68 (d, J = 8.4 Hz, 1H), 7.55 (dd, J = 8.4 Hz, 4,4 Hz, 1H), 7.41 (d, J = 8.4 Hz, 1H), 7.09 (d, J = 1.2 Hz, 1H), 5.39 (t, J = 9.6 Hz, 1H), 2.29- 2.16 (m, 1H), 1.05 (d, J = 6.8 Hz, 3H), 0.77 (d, J = |
| | | 6.4 Hz, 3H) |
| 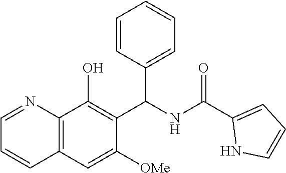 [SMILES: COc1cc2cccnc2c(O)c1C(NC(=O)c1ccc[nH]1)c1ccccc1] | 4 | N-((8-hydroxy-6-methoxyquinolin-7- yl)(phenyl)methyl)-1H-pyrrole-2-carboxamide LC-MS: m/z 374 (M + H)⁺. 1H NMR (400 MHz, CD3OD) δ (ppm): 8.67 (dd, J = 4.4 Hz, 1.6 Hz, 1H), 8.18 (d, J = 8.4 Hz, 1H), 7.46 (dd, J = 8.0 Hz, 4.0 Hz, 1H), 7.37-7.33 (m, 2H), 7.27-7.22 (m, 2H), 7.21-7.15 (m, 2H), 6.95- 6.90 (m, 2H), 6.79 (dd, J = 4.0 Hz, 1.6 Hz, 1H), 6.20 (dd, J = 3.6 Hz, 2.8 Hz, 1H), 3.98 (s, 3H) |
| 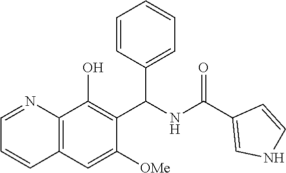 [SMILES: COc1cc2cccnc2c(O)c1C(NC(=O)c1cc[nH]c1)c1ccccc1] | 4 | N-((8-hydroxy-6-methoxyquinolin-7- yl)(phenyl)methyl)-1H-pyrrole-3-carboxamide LC-MS: m/z 374 (M + H)⁺. 1H NMR (400 MHz, DMSO-d6) δ (ppm): 11.28 (br s, 1H), 8.73 (dd, J = 4.4 Hz, 1.2 Hz, 1H), 8.71 (d, J = 8.4 Hz, 1.2 Hz, 1H), 8.17 (d, J = 8.4 Hz, 1H), 7.57 (dd, J = 8.4 Hz, 4.4 Hz, 1H), 7.37-7.25 (m, 5H), 7.24-7.21 (m, 1H), 7.16 (d, J = 8.4 Hz, 1H), 7.00 (s, 1H), 6.82 (d, J = 2.4 Hz, 1H), 6.42 (d, J = 2.0 Hz, 1H), 3.96 (s, 3H) |
| 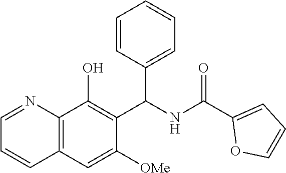 [SMILES: COc1cc2cccnc2c(O)c1C(NC(=O)c1ccco1)c1ccccc1] | 4 | N-((8-hydroxy-6-methoxyquinolin-7- yl)(phenyl)methyl)furan-2-carboxamide LC-MS: m/z 375 (M + H)⁺. 1H NMR (400 MHz, DMSO-d6) δ (ppm): 8.74 (dd, J = 4.4 Hz, 1.2 Hz, 1H), 8.63 (d, J = 9.6 Hz, 1H), 8.29 (dd, J = 8.4 Hz, 1.6 Hz, 1H), 7.90 (s, 1H), 7.58 (dd, J = 8.4 Hz, 4.4 Hz, 1H), 7.31-7.28 (m, 4H), 7.26-7.17 (m, 2H), 7.12 (d, J = 8.4 Hz, 1H), 7.04 (s, 1H), 6.68 (dd, J = 3.2 Hz, 1.6 Hz, 1H), 3.97 (s, 3H) |
| 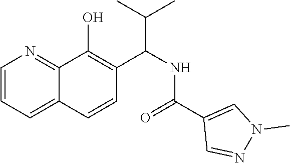 [SMILES: CC(C)C(NC(=O)c1cnn(C)c1)c1ccc2cccnc2c1O] | 5 | N-(1-(8-hydroxyquinolin-7-yl)-2-methylpropyl)-1- methyl-1H-pyrazole-4-carboxamide LC-MS: m/z 325 (M + H)⁺. 1H NMR (400 MHz, DMSO-d6) δ (ppm): 8.85 (d, J = 4.4 Hz, 1H), 8.31 (d, J = 9.6 Hz, 1H), 8.19 (s, 1H), 8.15 (d, J = 8.4 Hz, 1H), 7.88 (s, 1H), 7.66 (d, J = 8.4 Hz, 1H), 7.54 (dd, J = 8.4 Hz, 4.4 Hz, 1H), 7.40 (d, J = 8.4 Hz, 1H), 5.40 (t, J = 10.0 Hz, 1H), 3.85 (s, 3H), 2.18-2.06 (m, 1H), 1.02 (d, J = 6.8 Hz, 3H), 0.79 (d, J = 6.8 Hz, 3H) |
| 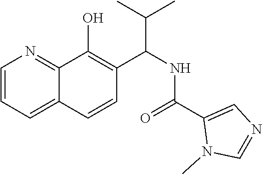 [SMILES: CC(C)C(NC(=O)c1cncn1C)c1ccc2cccnc2c1O] | 5 | N-(1-(8-hydroxyquinolin-7-yl)-2-methylpropyl)-1- methyl-1H-imidazole-5-carboxamide LC-MS: m/z 325 (M + H)⁺. 1H NMR (400 MHz, DMSO-d6) δ (ppm): 9.72 (br s, 1H), 8.85 (dd, J = 4.4 Hz, 1.2 Hz, 1H), 8.36 (dd, J = 9.6 Hz, 1H), 8.30 (dd, J = 8.4 Hz, 1.6 Hz, 1H), 7.71 (s, 2H), 7.66 (d, J = 8.4 Hz, 1H), 7.54 (dd, J = 8.4 Hz, 4.4 Hz, 1H), 7.41 (d, J = 8.4 Hz, 1H), 5.37 (t, J = 10.0 Hz, 1H), 3.75 (s, 3H), 2.24-2.12 (m, 1H), 1.04 (d, J = 6.8 Hz, 3H), 0.78 (d, J = 6.8 Hz, 3H) |
| 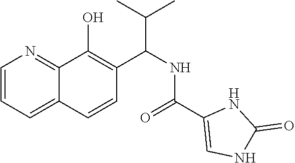 [SMILES: CC(C)C(NC(=O)c1c[nH]c(=O)[nH]1)c1ccc2cccnc2c1O] | 5 | N-(1-(8-hydroxyquinolin-7-yl)-2-methylpropyl)-2- oxo-2,3-dihydro-1H-imidazole-4-carboxamide LC-MS: m/z 327 (M + H)⁺. 1H NMR (400 MHz, DMSO-d6) δ (ppm): 10.28 (br s, 2H), 9.67 (br s, 1H), 8.86 (dd, J = 4.4 Hz, 1.2 Hz, 1H), 8.31 (dd, J = 8.4 Hz, 1.6 Hz, 1H), 7.89 (d, J = 9.2 Hz, 1H), 7.58-7.52 (m, 2H), 7.41 (d, J = 8.4 Hz, 1H), 7.12 (s, 1H), 5.34 (t, J = 9.6 Hz, 1H), 2.19-2.09 (m, 1H), 1.00 (d, J = 6.8 Hz, 3H), 0.80 (d, J = 6.8 Hz, 3H) |
| 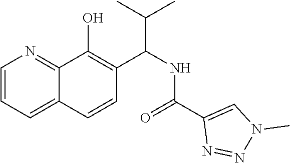 [SMILES: CC(C)C(NC(=O)c1cn(C)nn1)c1ccc2cccnc2c1O] | 5 | N-(1-(8-hydroxyquinolin-7-yl)-2-methylpropyl)-1- methyl-1H-1,2,3-triazole-4-carboxamide LC-MS: m/z 326 (M + H)⁺. 1H NMR (400 MHz, DMSO-d6) δ (ppm): 8.97 (d, J = 4.8 Hz, 1H), 8.52 (d, J = 8.4 Hz, 1H), 8.21 (d, J = 9.6 Hz, 1H), 8.03 (s, 1H), 7.65 (dd, J = 8.4 Hz, 4.4 Hz, 1H), 7.61 (dd, J = 8.4 Hz, 4.4 Hz, 1H), 7.46 (d, J = 8.4 Hz, 1H), 5.14 (t, J = 9.6 Hz, 1H), 4.06 (s, 3H), 2.52-2.43 (m, 1H), 1.10 (d, J = 6.8 Hz, 3H), 0.82 (d, J = 6.8 Hz, 3H) |
| 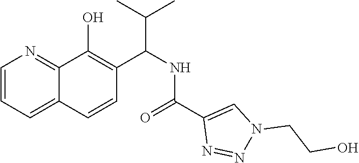 [SMILES: CC(C)C(NC(=O)c1cn(CCO)nn1)c1ccc2cccnc2c1O] | 5 | 1-(2-hydroxyethyl)-N-(1-(8-hydroxyquinolin-7-yl)- 2-methylpropyl)-1H-1,2,3-triazole-4-carboxamide LC-MS: m/z 356 (M + H)⁺. 1H NMR (400 MHz, DMSO-d6) δ (ppm): 8.86 (dd, J = 4.4 Hz, 1.6 Hz, 1H), 8.71 (d, J = 9.6 Hz, 1H), 8.47 (s, 1H), 8.30 (dd, J = 8.4 Hz, 1.6 Hz, 1H), 7.68 (d, J = 8.4 Hz, 1H), 7.54 (dd, J = 8.4 Hz, 4.4 Hz, 1H), 7.40 (d, J = 8.4 Hz, 1H), 5.29 (t, J = 9.6 Hz, 1H), 5.02 (t, J = 4.0 Hz, 1H), 4.44 (t, J = 4.8 Hz, 2H), 3.81-3.76 (m, 2H), 2.34-2.27 (m, 1H), |
| | | 1.03 (d, J = 6.8 Hz, 3H), 0.77 (d, J = 6.4 Hz, 3H) |
| 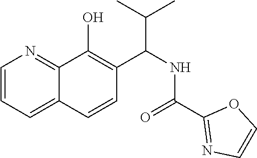 [SMILES: CC(C)C(NC(=O)c1ncco1)c1ccc2cccnc2c1O] | 5 | N-(1-(8-hydroxyquinolin-7-yl)-2- methylpropyl)oxazole-2-carboxamide LC-MS: m/z 312 (M + H)⁺. 1H NMR (400 MHz, DMSO-d6) δ (ppm): 9.13 (d, J = 9.2 Hz, 1H), 8.85 (dd, J = 4.4 Hz, 1.2 Hz, 1H), 8.32-8.28 (m, 2H), 7.70 (d, J = 8.8 Hz, 1H), 7.55 (dd, J = 8.4 Hz, 4,4 Hz, 1H), 7.45 (s, 1H), 7.41 (d, J = 8.8 Hz, 1H), 5.29 (t, J = 9.6 Hz, 1H), 2.34-2.26 (m, 1H), 1.04 (d, J = 6.8 Hz, 3H), 0.76 (d, J = 6.4 Hz, 3H) |
| 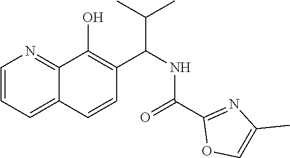 [SMILES: Cc1coc(C(=O)NC(c2ccc3cccnc3c2O)C(C)C)n1] | 5 | N-(1-(8-hydroxyquinolin-7-yl)-2-methylpropyl)-4- methyloxazole-2-carboxamide LC-MS: m/z 326 (M + H)⁺. 1H NMR (400 MHz, CDCl3) δ (ppm): 8.71 (dd, J = 4.4 Hz, 1.6 Hz, 1H), 8.29 (d, J = 9.2 Hz, 1H), 8.07 (dd, J = 8.4 Hz, 1.2 Hz, 1H), 7.45 (s, 1H), 7.42-7.34 (m, 2H), 7.27 (d, J = 8.4 Hz, 1H), 5.03 (t, J = 9.2 Hz, 1H), 2.46-2.35 (m, 1H), 2.15 (s, 3H), 1.05 (d, J = 6.8 Hz, 3H), 0.81 (d, J = 6.8 Hz, 3H) |
| 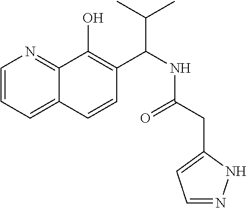 [SMILES: CC(C)C(NC(=O)Cc1ccn[nH]1)c1ccc2cccnc2c1O] | 5 | N-(1-(8-hydroxyquinolin-7-yl)-2-methylpropyl)-2- (1H-pyrazol-5-yl)acetamide LC-MS: m/z 325 (M + H)⁺. 1H NMR (400 MHz, DMSO-d6) δ (ppm): 12.53 (br s, 1H), 9.68 (br s, 1H), 8.84 (dd, J = 4.4 Hz, 1.2 Hz, 1H), 8.31-8.27 (m, 2H), 7.59-7.50 (m, 3H), 7.39 (d, J = 8.4 Hz, 1H), 6.07 (br s, 1H), 5.24 (t, J = 10.4 Hz, 1H), 3.50 (s, 2H), 2.12-2.02 (m, 1H), 0.91 (d, J = 6.8 Hz, 3H), 0.78 (d, J = 6.8 Hz, 3H) |
| 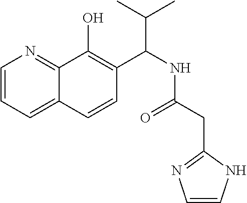 [SMILES: CC(C)C(NC(=O)Cc1ncc[nH]1)c1ccc2cccnc2c1O] | 5 | N-(1-(8-hydroxyquinolin-7-yl)-2-methylpropyl)-2- (1H-imidazol-2-yl)acetamide LC-MS: m/z 325 (M + H)⁺. 1H NMR (400 MHz, CD3OD) δ (ppm): 8.77 (dd, J = 4.0 Hz, 1.6 Hz, 1H), 8.25 (d, J = 8.4 Hz, 1H), 7.48 (dd, J = 8.4 Hz, 1.6 Hz, 1H), 7.45 (d, J = 8.4 Hz, 1H), 7.36 (d, J = 8.4 Hz, 1H), 6.94 (s, 2H), 5.16 (d, J = 10.4 Hz, 1H), 3.31 (s, 2H), 2.31-2.22 (m, 1H), 1.02 (d, J = 6.8 Hz, 3H), 0.79 (d, J = 6.8 Hz, 3H) |
| 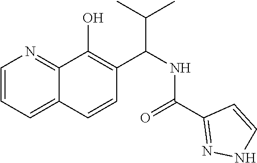 [SMILES: CC(C)C(NC(=O)c1cc[nH]n1)c1ccc2cccnc2c1O] | 5 | N-(1-(8-hydroxyquinolin-7-yl)-2-methylpropyl)- 1H-pyrazole-3-carboxamide LC-MS: m/z 311 (M + H)⁺. 1H NMR (400 MHz, DMSO-d6) δ (ppm): 13.24 (br s, 1H), 9.66 (br s, 1H), 8.66 (d, J = 4.4 Hz, 1H), 8.38 (d, J = 9.2 Hz, 1H), 8.30 (d, J = 8.4 Hz, 1H), 7.76 (s, 1H), 7.63 (d, J = 8.4 Hz, 1H), 7.54 (dd, J = 8.4 Hz, 4.4 Hz, 1H), 7.39 (d, J = 8.4 Hz, 1H), 6.63 (br s, 1H), 5.24 (br s, 1H), 2.32-2.25 (m, 1H), 1.02 (d, J = 6.8 Hz, 3H), 0.77 (d, J = 6.8 Hz, 3H) |
| 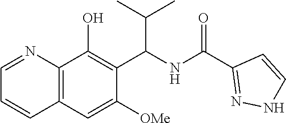 [SMILES: COc1cc2cccnc2c(O)c1C(NC(=O)c1cc[nH]n1)C(C)C] | 5 | N-(1-(8-hydroxy-6-methoxyquinolin-7-yl)-2- methylpropyl)-1H-pyrazole-3-carboxamide LC-MS: m/z 341 (M + H)⁺. 1H NMR (400 MHz, CD3OD) δ (ppm): 8.77 (d, J = 4.4 Hz, 1H), 8.65 (d, J = 8.8 Hz, 1H), 7.78 (dd, J = 8.4 Hz, 4.4 Hz, 1H), 7.69 (d, J = 2.0 Hz, 1H), 7.15 (s, 1H), 6.76 (d, J = 2.0 Hz, 1H), 5.47 (d, J = 10.4 Hz, 1H), 4.08 (s, 3H), 2.69-2.61 (m, 1H), 1.15 |
| | | (d, J = 6.8 Hz, 3H), 0.80 (d, J = 6.8 Hz, 3H) |
| 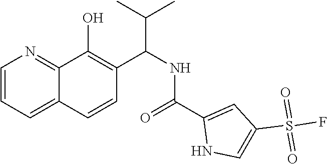 [SMILES: CC(C)C(NC(=O)c1cc(S(=O)(=O)F)c[nH]1)c1ccc2cccnc2c1O] | 5 | 5-((1-(8-hydroxyquinolin-7-yl)-2- methylpropyl)carbamoyl)-1H-pyrrole-3-sulfonyl fluoride LC-MS: m/z 392 (M + H)⁺. 1H NMR (400 MHz, DMSO-d6) δ (ppm): 12.99 (br s, 1H), 9.78 (br s, 1H), 8.86 (d, J = 4.4 Hz, 1H), 8.58 (d, J = 9.2 Hz, 1H), 8.30 (d, J = 8.4 Hz, 1H), 7.92 (s, 1H), 7.67-7.64 (m, 2H), 7.55 (dd, J = 8.4 Hz, 4.4 Hz, 1H), 7.42 (d, J = 8.4 Hz, 1H), 5.43 (t, J = 9.2 Hz, 1H), 2.25-2.15 (m, 1H), 1.04 (d, J = 6.8 Hz, 3H), 0.81 (d, J = 6.8 Hz, 3H) |
| 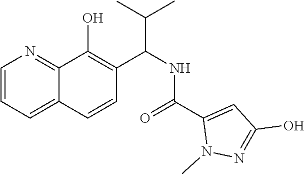 [SMILES: CC(C)C(NC(=O)c1cc(O)nn1C)c1ccc2cccnc2c1O] | 5 | 3-hydroxy-N-(1-(8-hydroxyquinolin-7-yl)-2- methylpropyl)-1-methyl-1H-pyrazole-5- carboxamide LC-MS: m/z 341 (M + H)⁺. 1H NMR (400 MHz, DMSO-d6) δ (ppm): 9.69 (br s, 1H), 8.86 (dd, J = 4.4 Hz, 1.6 Hz, 1H), 8.48 (d, J = 9.2 Hz, 1H), 8.33 (d, J = 8.4 Hz, 1.6 Hz, 1H), 7.66 (d, J = 8.4 Hz, 1H), 7.55 (dd, J = 8.4 Hz, 4.4 Hz, 1H), 7.41 (d, J = 8.4 Hz, 1H), 6.18 (s, 1H), 5.36 (t, J = 9.2 Hz, 1H), 3.76 (s, 3H), 2.23-2.11 (m, 1H), 1.04 (d, J = 6.8 Hz, 3H), 0.77 (d, J = 6.8 Hz, |
| | | 3H) |
| 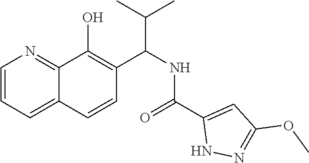 [SMILES: COc1cc(C(=O)NC(c2ccc3cccnc3c2O)C(C)C)[nH]n1] | 5 | N-(1-(8-hydroxyquinolin-7-yl)-2-methylpropyl)-3- methoxy-1H-pyrazole-5-carboxamide LC-MS: m/z 341 (M + H)⁺. 1H NMR (400 MHz, DMSO-d6) δ (ppm): 12.60 (br s, 1H), 9.55 (br s, 1H), 8.86 (d, J = 4.4 Hz, 1H), 8.46 (br s, 1H), 8.31 (d, J = 9.2 Hz, 1H), 7.65 (d, J = 8.4 Hz, 1H), 7.55 (dd, J = 8.4 Hz, 4.4 Hz, 1H), 7.42 (d, J = 8.4 Hz, 1H), 6.43 (br s, 1H), 5.39 (br s, 1H), 3.79 (s, 3H), 2.24-2.13 (m, 1H), 1.03 (d, J = 6.8 Hz, 3H), 0.79 (d, J = 6.8 Hz, 3H) |
| 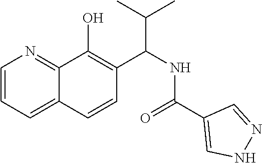 [SMILES: CC(C)C(NC(=O)c1cn[nH]c1)c1ccc2cccnc2c1O] | 5 | N-(1-(8-hydroxyquinolin-7-yl)-2-methylpropyl)- 1H-pyrazole-4-carboxamide LC-MS: m/z 311 (M + H)⁺. 1H NMR (400 MHz, CD3OD) δ (ppm): 8.80 (dd, J = 4.0 Hz, 1.2 Hz, 1H), 8.22 (dd, J = 8.4 Hz, 1.6 Hz, 1H), 8.09 (s, 2H), 7.53 (d, J = 8.4 Hz, 1H), 7.47 (dd, J = 8.4 Hz, 4.0 Hz, 1H), 7.38 (d, J = 8.4 Hz, 1H), 5.29 (d, J = 10.0 Hz, 1H), 2.42-2.35 (m, 1H), 1.14 (d, J = 6.8 Hz, 3H), 0.85 (d, J = 6.8 Hz, 3H) |
| 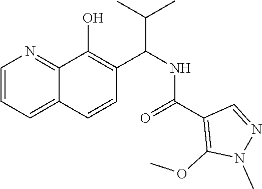 [SMILES: COc1c(C(=O)NC(c2ccc3cccnc3c2O)C(C)C)cnn1C] | 5 | N-(1-(8-hydroxyquinolin-7-yl)-2-methylpropyl)-5- methoxy-1-methyl-1H-pyrazole-4-carboxamide LC-MS: m/z 355 (M + H)⁺. 1H NMR (400 MHz, CD3OD) δ (ppm): 8.98 (dd, J = 4.8 Hz, 1.2 Hz, 1H), 8.88 (d, J = 8.4 Hz, 1H), 7.90 (dd, J = 8.4 Hz, 5.2 Hz, 1H), 7.82-7.71 (m, 3H), 5.09 (d, J = 10.4 Hz, 1H), 4.03 (s, 3H), 3.66 (s, 3H), 2.58-2.50 (m, 1H), 1.21 (d, J = 6.4 Hz, 3H), 0.84 (d, J = 6.4 Hz, 3H) |
| 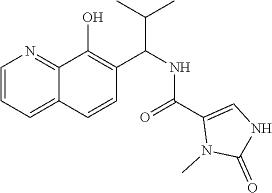 [SMILES: CC(C)C(NC(=O)c1c[nH]c(=O)n1C)c1ccc2cccnc2c1O] | 5 | N-(1-(8-hydroxyquinolin-7-yl)-2-methylpropyl)-3- methyl-2-oxo-2,3-dihydro-1H-imidazole-4- carboxamide LC-MS: m/z 341 (M + H)⁺. 1H NMR (400 MHz, CD3OD) δ (ppm): 9.00 (dd, J = 4.4 Hz, 1.2 Hz, 1H), 8.94 (d, J = 8.0 Hz, 1H), 7.94 (dd, J = 8.4 Hz, 4.8 Hz, 1H), 7.81 (d, J = 8.4 Hz, 1H), 7.78 (d, J = 8.4 Hz, 1H), 7.24 (s, 1H), 5.10 (t, J = 10.4 Hz, 1H), 3.48 (s, 3H), 2.49-2.40 (m, 1H), 1.20 (d, J = 6.8 Hz, 3H), 0.82 (d, J = 6.8 Hz, 3H) |
| 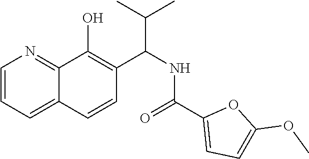 [SMILES: COc1ccc(C(=O)NC(c2ccc3cccnc3c2O)C(C)C)o1] | 5 | N-(1-(8-hydroxyquinolin-7-yl)-2-methylpropyl)-5- methoxyfuran-2-carboxamide LC-MS: m/z 341 (M + H)⁺. 1H NMR (400 MHz, DMSO-d6) δ (ppm): 9.86 (br s, 1H), 8.86 (d, J = 4.4 Hz, 1.2 Hz, 1H), 8.30 (dd, J = 8.4 Hz, 1.6 Hz, 1H), 8.20 (d, J = 9.2 Hz, 1H), 7.67 (d, J = 8.4 Hz, 1H), 7.56 (dd, J = 8.4 Hz, 4.4 Hz, 1H), 7.40 (d, J = 8.4 Hz, 1H), 7.06 (d, J = 4.0 Hz, 1H), 5.54 (d, J = 4.0 Hz, 1H), 5.29 (t, J = 9.2 Hz, 1H), 3.80 (s, 3H), 2.26-2.17 (m, 1H), 1.02 (d, J = |
| | | 6.8 Hz, 3H), 0.76 (d, J = 6.8 Hz, 3H) |
| 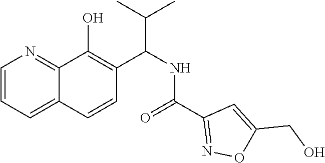 [SMILES: CC(C)C(NC(=O)c1cc(CO)on1)c1ccc2cccnc2c1O] | 5 | 5-(hydroxymethyl)-N-(1-(8-hydroxyquinolin-7-yl)- 2-methylpropyl)isoxazole-3-carboxamide LC-MS: m/z 342 (M + H)⁺. 1H NMR (400 MHz, DMSO-d6) δ (ppm): 9.88 (br s, 1H), 8.99 (d, J = 9.2 Hz 1H), 8.87 (dd, J = 4.4 Hz, 1H), 8.31 (d, J = 8.4 Hz, 1H), 7.69 (d, J = 8.4 Hz, 1H), 7.56 (dd, J = 8.4 Hz, 4.4 Hz, 1H), 7.41 (d, J = 8.4 Hz, 1H), 6.64 (s, 1H), 5.72 (t, J = 6.0 Hz, 1H), 5.36 (t, J = 9.2 Hz, 1H), 4.61 (d, J = 6.0 Hz, 2H), 2.24-2.20 (m, 1H), 1.05 (d, J = 6.8 Hz, 3H), |
| | | 0.77 (d, J = 6.8 Hz, 3H) |
| 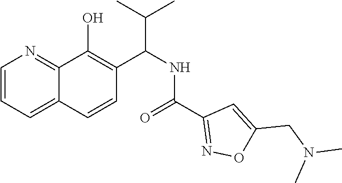 [SMILES: CC(C)C(NC(=O)c1cc(CN(C)C)on1)c1ccc2cccnc2c1O] | 5 | 5-((dimethylamino)methyl)-N-(1-(8- hydroxyquinolin-7-yl)-2-methylpropyl)isoxazole- 3-carboxamide LC-MS: m/z 369 (M + H)⁺. 1H NMR (400 MHz, DMSO-d6) δ (ppm): 9.86 (br s, 1H), 8.99 (d, J = 8.8 Hz 1H), 8.86 (dd, J = 4.4 Hz, 1.6 Hz, 1H), 8.31 (dd, J = 8.4 Hz, 1.2 Hz, 1H), 7.69 (d, J = 8.4 Hz, 1H), 7.56 (dd, J = 8.4 Hz, 4.4 Hz, 1H), 7.41 (d, J = 8.4 Hz, 1H), 6.67 (s, 1H), 5.36 (t, J = 9.2 Hz, 1H), 3.66 (s, 2H), 2.28-2.18 (m, 1H), 2.23 (s, 6H), 1.05 (d, J = 6.8 Hz, 3H), 0.77 (d, J = 6.8 Hz, 3H) |
| 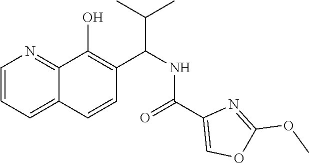 [SMILES: COc1nc(C(=O)NC(c2ccc3cccnc3c2O)C(C)C)co1] | 5 | N-(1-(8-hydroxyquinolin-7-yl)-2-methylpropyl)-2- methoxyoxazole-4-carboxamide LC-MS: m/z 342 (M + H)⁺. 1H NMR (400 MHz, DMSO-d6) δ (ppm): 8.87 (d, J = 4.4 Hz, 1.6 Hz 1H), 8.34-8.30 (m, 2H), 8.14 (s, 1H), 7.60 (d, J = 8.4 Hz, 1H), 7.56 (dd, J = 8.4 Hz, 4.4 Hz, 1H), 7.41 (d, J = 8.4 Hz, 1H), 5.17 (t, J = 9.2 Hz, 1H), 4.11 (s, 3H), 2.36-2.27 (m, 1H), 1.01 (d, J = 6.8 Hz, 3H), 0.77 (d, J = 6.8 Hz, 3H) |
| 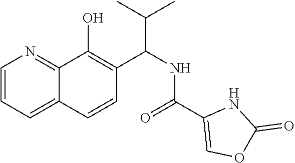 [SMILES: CC(C)C(NC(=O)c1coc(=O)[nH]1)c1ccc2cccnc2c1O] | 5 | N-(1-(8-hydroxyquinolin-7-yl)-2-methylpropyl)-2- oxo-2,3-dihydrooxazole-4-carboxamide LC-MS: m/z 328 (M + H)⁺. 1H NMR (400 MHz, DMSO-d6) δ (ppm): 8.87 (d, J = 4.4 Hz, 1.6 Hz 1H), 8.32 (d, J = 8.4 Hz, 1H), 8.30 (d, J = 8.8 Hz, 1H), 7.78 (s, 1H), 7.57-7.52 (m, 2H), 7.42 (d, J = 8.4 Hz, 1H), 5.33 (t, J = 9.2 Hz, 1H), 2.20-2.13 (m, 1H), 1.01 (d, J = 6.8 Hz, 3H), 0.80 (d, J = 6.8 Hz, 3H) |
| 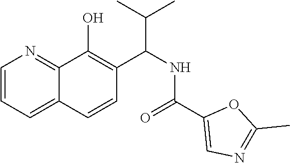 [SMILES: Cc1ncc(C(=O)NC(c2ccc3cccnc3c2O)C(C)C)o1] | 5 | N-(1-(8-hydroxyquinolin-7-yl)-2-methylpropyl)-2- methyloxazole-5-carboxamide LC-MS: m/z 326 (M + H)⁺. 1H NMR (400 MHz, CD3OD) δ (ppm): 8.99 (d, J = 4.4 Hz, 1.6 Hz 1H), 8.90 (dd, J = 8.4 Hz, 1.2 Hz, 1H), 7.93 (dd, J = 8.4 Hz, 4.8 Hz, 1H), 7.82 (d, J = 8.8 Hz, 1H), 7.75 (d, J = 8.8 Hz, 1H), 7.61 (s, 1H), 5.20 (d, J = 10.4 Hz, 1H), 2.53 (s, 3H), 2.55- 2.46 (m, 1H), 1.21 (d, J = 6.8 Hz, 3H), 0.84 (d, J = 6.8 Hz, 3H) |
| 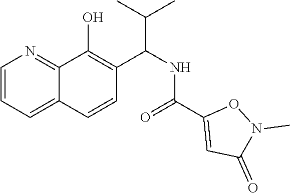 [SMILES: CC(C)C(NC(=O)c1cc(=O)n(C)o1)c1ccc2cccnc2c1O] | 5 | N-(1-(8-hydroxyquinolin-7-yl)-2-methylpropyl)-2- methyl-3-oxo-2,3-dihydroisoxazole-5-carboxamide LC-MS: m/z 342 (M + H)⁺. 1H NMR (400 MHz, CD3OD) δ (ppm): 8.98 (d, J = 4.4 Hz, 1.6 Hz 1H), 8.82 (d, J = 8.0 Hz, 1H), 7.88 (dd, J = 8.4 Hz, 4.4 Hz, 1H), 7.75 (d, J = 8.4 Hz, 1H), 7.70 (d, J = 8.4 Hz, 1H), 6.40 (s, 1H), 5.25 (d, J = 10.4 Hz, 1H), 3.61 (s, 3H), 2.46-2.39 (m, 1H), 1.19 (d, J = 6.8 Hz, 3H), 0.83 (d, J = 6.8 Hz, 3H) |
| 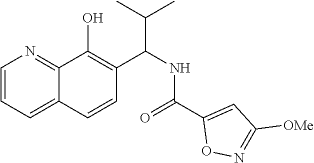 [SMILES: COc1cc(C(=O)NC(c2ccc3cccnc3c2O)C(C)C)on1] | 5 | N-(1-(8-hydroxyquinolin-7-yl)-2-methylpropyl)-3- methoxyisoxazole-5-carboxamide LC-MS: m/z 342 (M + H)⁺. 1H NMR (400 MHz, DMSO-d6) δ (ppm): 9.85 (br s, 1H), 9.10 (d, J = 8.4 Hz, 1H), 8.86 (d, J = 1.6 Hz, 1H), 8.31 (d, J = 8.8 Hz, 1H), 7.68 (d, J = 8.4 Hz, 1H), 7.56 (dd, J = 8.4 Hz, 4.4 Hz, 1H), 7.42 (d, J = 8.4 Hz, 1H), 6.85 (s, 1H), 5.36 (t, J = 8.4 Hz, 1H), 3.94 (s, 3H), 2.29-2.18 (m, 1H), 1.05 (d, J = 6.8 Hz, 3H), 0.77 (d, J = 6.8 Hz, 3H) |
| 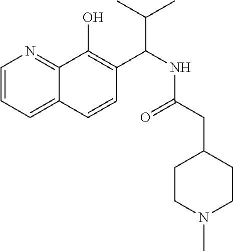 [SMILES: CC(C)C(NC(=O)CC1CCN(C)CC1)c1ccc2cccnc2c1O] | 5 | N-(1-(8-hydroxyquinolin-7-yl)-2-methylpropyl)-2- (1-methylpiperidin-4-yl)acetamide LC-MS: m/z 356 (M + H)⁺. 1H NMR (400 MHz, CD3OD) δ (ppm): 8.80 (dd, J = 4.4 Hz, 1.6 Hz, 1H), 8.21 (dd, J = 8.4 Hz, 1.6 Hz, 1H), 7.46 (dd, J = 8.0 Hz, 4.0 Hz, 1H), 7.43 (d, J = 8.4 Hz, 1H), 7.35 (d, J = 8.8 Hz, 1H), 5.15 (d, J = 9.2 Hz, 1H), 2.89-2.83 (m, 2H), 2.28 (s, 3H), 2.30- 2.26 (m, 1H), 2.18 (d, J = 6.4 Hz, 2H), 2.12-1.95 (m, 2H), 1.78-1.71 (m, 2H), 1.65-1.56 (m, 1H), 1.38-1.26 (m, 2H), 1.06 (d, J = 6.8 Hz, 3H), 0.83 (d, J = 6.8 Hz, 3H) |
| 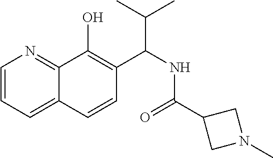 [SMILES: CC(C)C(NC(=O)C1CN(C)C1)c1ccc2cccnc2c1O] | 5 | N-(1-(8-hydroxyquinolin-7-yl)-2-methylpropyl)-1- methylazetidine-3-carboxamide LC-MS: m/z 314 (M + H)⁺. 1H NMR (400 MHz, CD3OD) δ (ppm): 8.80 (dd, J = 4.4 Hz, 1.6 Hz, 1H), 8.22 (dd, J = 8.4 Hz, 1.6 Hz, 1H), 7.47 (dd, J = 8.4 Hz, 4.4 Hz, 1H), 7.41 (d, J = 8.4 Hz, 1H), 7.35 (d, J = 8.4 Hz, 1H), 5.16 (d, J = 9.2 Hz, 1H), 3.70-3.56 (m, 2H), 3.47-3.40 (m, 2H), 3.38-3.22 (m, 1H), 2.35(s, 3H), 2.33-2.23 (m, 1H), 1.06 (d, J = 6.8 Hz, 3H), 0.80 (d, J = 6.8 Hz, 3H) |
| 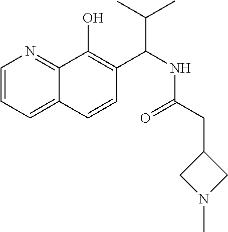 [SMILES: CC(C)C(NC(=O)CC1CN(C)C1)c1ccc2cccnc2c1O] | 5 | N-(1-(8-hydroxyquinolin-7-yl)-2-methylpropyl)-2- (1-methylazetidin-3-yl)acetamide LC-MS: m/z 328 (M + H)⁺. 1H NMR (400 MHz, DMSO-d6) δ (ppm): 9.67 (br s, 1H), 8.84 (dd, J = 8.4 Hz, 1.6 Hz, 1H), 8.29 (dd, J = 8.4 Hz, 1.6 Hz, 1H), 8.16 (d, J = 9.2 Hz, 1H), 7.53 (dd, J = 8.4 Hz, 4.4 Hz, 1H), 7.50 (d, J = 8.8 Hz, 1H), 7.39 (d, J = 8.4 Hz, 1H), 5.23 (t, J = 8.4 Hz, 1H), 3.25-3.14 (m, 2H), 2.78-2.68 (m, 2H), 2.59-2.46 (m, 1H), 2.45-2.30 (m, 2H), 2.13 (s, 3H), 2.09-1.93 (m, 1H), 0.91 (d, J = 6.4 Hz, 3H), 0.77 (d, J = 6.8 Hz, 3H) |
| 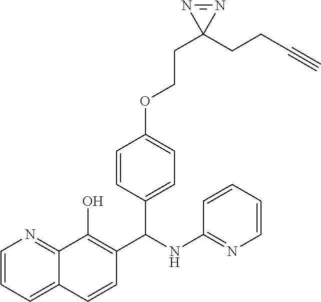 [SMILES: C#CCCC1(CCOc2ccc(C(Nc3ccccn3)c3ccc4cccnc4c3O)cc2)N=N1] | 2 | 7-((4-(2-(3-(but-3-yn-1-yl)-3H-diazirin-3- yl)ethoxy)phenyl)(pyridin-2- ylamino)methyl)quinolin-8-ol LC-MS: m/z 464 (M + H)⁺. 1H NMR (400 MHz, DMSO-d6) δ 9.89 (brs, 1H), 8.84 (dd, J = 4.2, 1.6 Hz, 1H), 8.28 (dd, J = 8.3, 1.6 Hz, 1H), 7.90 (dd, J = 5.2, 1.8 Hz, 1H), 7.62 (d, J = 8.5 Hz, 1H), 7.52 (dd, J = 8.3, 4.2 Hz, 1H), 7.42- 7.28 (m, 3H), 7.24 (d, J = 8.7 Hz, 2H), 6.86-6.80 (m, 2H), 6.76 (d, J = 8.3 Hz, 1H), 6.65 (d, J = 8.4 Hz, 1H), 6.45 (dd, J = 6.9, 5.2 Hz, 1H), 3.75 (t, J = 6.0 Hz, 2H), 2.82 (t, J = 2.7 Hz, 1H), 2.01 (td, J = 7.4, 2.7 Hz, 2H), 1.84 (t, J = 6.0 Hz, 2H), 1.63 (t, J = 7.4 Hz, 2H). |
| 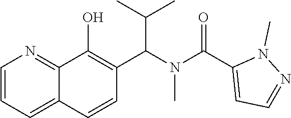 [SMILES: CC(C)C(c1ccc2cccnc2c1O)N(C)C(=O)c1ccnn1C] | 5 | N-(1-(8-hydroxyquinolin-7-yl)-2-methylpropyl)- N,1-dimethyl-1H-pyrazole-5-carboxamide LC-MS: m/z 339 (M + H)⁺. 1H NMR (400 MHz, CD3OD) mixture of rotamers (ratio ~3:2) δ (ppm): 8.85-8.79 (m, 1H), 8.30-8.23 (m, 1H), 7.72-7.27 (m, 4H), 6.80 (minor) and 6.40 (major) (two sets of s, 1H), 5.90 (major) and 5.40 (minor) (two sets of d, J = 12 Hz, 1H), 3.85 (major) |
| | | and 3.79 (minor) (two sets of s, 3H), 2.99 (minor) |
| | | and 2.85 (major) (two sets of s, 3H), 2.97-2.78 (m, |
| | | 1H), 1.18 (major) and 1.16 (minor) (two sets of d, J = |
| | | 6.8 Hz, 3H), 0.99 (major) and 0.79 (minor) (two |
| | | sets of d, J = 6.8 Hz, 3H) |
| 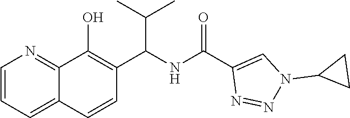 [SMILES: CC(C)C(NC(=O)c1cn(C2CC2)nn1)c1ccc2cccnc2c1O] | 5 | 1-cyclopropyl-N-(1-(8-hydroxyquinolin-7-yl)-2- methylpropyl)-1H-1,2,3-triazole-4-carboxamide LC-MS: m/z 352 (M + H)⁺. 1H NMR (400 MHz, CD3OD) δ (ppm): 8.84 (d, J = 2.4 Hz, 1H), 8.35 (s, 1H), 8.24 (d, J = 8.0 Hz, 1H), 7.52-7.45 (m, 2H), 7.35 (d, J = 8.4 Hz, 1H), 5.20 (d, J = 9.2 Hz, 1H), 4.00-3.91 (m, 1H), 2.52- 2.39 (m, 1H), 1.28-1.23 (m, 2H), 1.22-1.13 (m, |
| | | 2H), 1.11 (d, J = 6.8 Hz, 3H), 0.85 (d, J = 6.8 Hz, |
| | | 3H) |
| 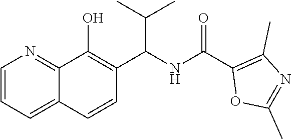 [SMILES: Cc1nc(C)c(C(=O)NC(c2ccc3cccnc3c2O)C(C)C)o1] | 5 | N-(1-(8-hydroxyquinolin-7-yl)-2-methylpropyl)- 2,4-dimethyloxazole-5-carboxamide LC-MS: m/z 340 (M + H)⁺. 1H NMR (400 MHz, CD3OD) δ (ppm): 8.83 (dd, J = 8.4 Hz, 1.6 Hz, 1H), 8.29 (d, J = 8.4 Hz, 1H), 7.55-7.49 (m, 2H), 7.40 (d, J = 8.4 Hz, 1H), 5.18 (d, J = 9.6 Hz, 1H), 2.50 (s, 3H), 2.47-2.37 (m, 1H), 2.36 (s, 3H), 1.13 (d, J = 6.8 Hz, 3H), 0.85 (d, J = 6.8 Hz, 3H) |
| 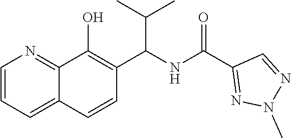 [SMILES: CC(C)C(NC(=O)c1cnn(C)n1)c1ccc2cccnc2c1O] | 5 | N-(1-(8-hydroxyquinolin-7-yl)-2-methylpropyl)-2- methyl-2H-1,2,3-triazole-4-carboxamide LC-MS: m/z 326 (M + H)⁺. 1H NMR (400 MHz, CD3OD) δ (ppm): 8.84 (dd, J = 8.4 Hz, 1.6 Hz, 1H), 8.31 (d, J = 8.4 Hz, 1H), 7.99 (s, 1H), 7.57-7.50 (m, 2H), 7.42 (d, J = 8.4 Hz, 1H), 5.20 (d, J = 10.0 Hz, 1H), 4.23 (s, 3H), 2.51- 2.39 (m, 1H), 1.12 (d, J = 6.4 Hz, 3H), 0.86 (d, J = 6.8 Hz, 3H) |
| 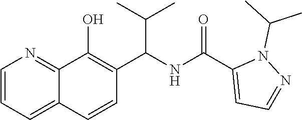 [SMILES: CC(C)C(NC(=O)c1ccnn1C(C)C)c1ccc2cccnc2c1O] | 5 | N-(1-(8-hydroxyquinolin-7-yl)-2-methylpropyl)-1- isopropyl-1H-pyrazole-5-carboxamide LC-MS: m/z 353 (M + H)⁺. 1H NMR (400 MHz, CD3OD) δ (ppm): 8.82 (dd, J = 8.4 Hz, 1.6 Hz, 1H), 8.27 (dd, J = 8.4 Hz, 1.2 Hz 1H), 7.55-7.48 (m, 3H), 7.40 (d, J = 8.4 Hz, 1H), 6.73 (d, J = 2.0 Hz, 1H), 5.33-5.21 (m, 2H), 2.44- 2.29 (m, 1H), 1.42 (d, J = 6.4 Hz, 3H), 1.36 (d, J = |
| | | 6.8 Hz, 3H), 1.15 (d, J = 6.8 Hz, 3H), 0.86 (d, J = |
| | | 6.8 Hz, 3H) |
| 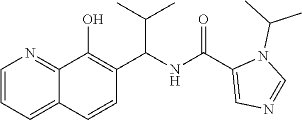 [SMILES: CC(C)C(NC(=O)c1cncn1C(C)C)c1ccc2cccnc2c1O] | 5 | N-(1-(8-hydroxyquinolin-7-yl)-2-methylpropyl)-1- isopropyl-1H-imidazole-5-carboxamide LC-MS: m/z 353 (M + H)⁺. 1H NMR (400 MHz, DMSO-d6) δ (ppm): 9.72 (br s, 1H), 8.85 (d, J = 2.8 Hz, 1H), 8.46 (d, J = 8.8 Hz, 1H), 8.29 (d, J = 7.6 Hz, 1H), 7.94 (s-1H), 7.66 (d, J = 8.4 Hz, 1H), 7.61 (s, 1H), 7.53 (dd, J = 8.4 Hz, 4.0 Hz, 1H), 7.41 (d, J = 8.4 Hz, 1H), 5.37 (t, J = |
| | | 9.6 Hz, 1H), 5.08 (hept, J = 6.8 Hz, 1H), 2.24- |
| | | 2.09 (m, 1H), 1.37 (d, J = 6.8 Hz, 3H), 1.30 (d, J = |
| | | 6.8 Hz, 3H), 1.04 (d, J = 6.4 Hz, 3H), 0.78 (d, J = |
| | | 6.8 Hz, 3H) |
| 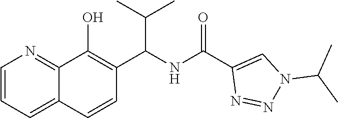 [SMILES: CC(C)C(NC(=O)c1cn(C(C)C)nn1)c1ccc2cccnc2c1O] | 5 | N-(1-(8-hydroxyquinolin-7-yl)-2-methylpropyl)-1- isopropyl-1H-1,2,3-triazole-4-carboxamide LC-MS: m/z 354 (M + H)⁺. 1H NMR (400 MHz, DMSO-d6) δ (ppm): 9.99 (br s, 1H), 8.86 (dd, J = 4.4 Hz, 1.6 Hz, 1H), 8.70 (d, J = 9.6 Hz, 1H), 8.62 (s, 1H), 8.32 (dd, J = 8.4 Hz, 1.2 Hz, 1H), 7.68 (d, J = 8.4 Hz, 1H), 7.55 (dd, J = 8.4 Hz, 4.0 Hz, 1H), 7.40 (d, J = 8.4 Hz, 1H), 5.29 |
| | | (t, J = 9.6 Hz, 1H), 4.86 (hept, J = 6.8 Hz, 1H), |
| | | 2.39-2.26 (m, 1H), 1.49 (d, J = 6.8 Hz, 3H), 1.48 |
| | | (d, J = 6.8 Hz, 3H), 1.03 (d, J = 6.4 Hz, 3H), 0.77 |
| | | (d, J = 6.8 Hz, 3H) |
| 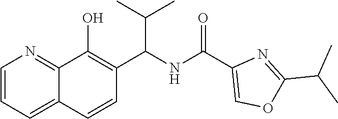 [SMILES: CC(C)c1nc(C(=O)NC(c2ccc3cccnc3c2O)C(C)C)co1] | 5 | N-(1-(8-hydroxyquinolin-7-yl)-2-methylpropyl)-2- isopropyloxazole-4-carboxamide LC-MS: m/z 354 (M + H)⁺. 1H NMR (400 MHz, CD3Cl) δ (ppm): 8.77 (dd, J = 4.4 Hz, 1.6 Hz, 1H), 8.21 (d, J = 9.6 Hz, 1H), 8.13 (dd, J = 8.0 Hz, 1.6 Hz, 1H), 8.04 (s, 1H), 7.43 (d, J = 8.4 Hz, 1H), 7.41 (dd, J = 8.4 Hz, 4.4 Hz, 1H), 7.30 (d, J = 8.4 Hz, 1H), 5.13 (t, J = 9.6 Hz, |
| | | 1H), 3.09 (hept, J = 6.8 Hz, 1H), 2.54-2.32 (m, |
| | | 1H), 1.35 (d, J = 6.8 Hz, 6H), 1.12 (d, J = 6.8 Hz, |
| | | 3H), 0.88 (d, J = 6.8 Hz, 3H) |
| 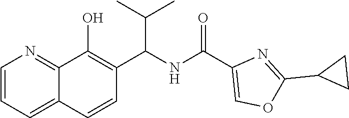 [SMILES: CC(C)C(NC(=O)c1coc(C2CC2)n1)c1ccc2cccnc2c1O] | 5 | 2-cyclopropyl-N-(1-(8-hydroxyquinolin-7-yl)-2- methylpropyl)oxazole-4-carboxamide LC-MS: m/z 352 (M + H)⁺. 1H NMR (400 MHz, CD3OD) δ (ppm): 8.83 (dd, J = 4.4 Hz, 1.2 Hz, 1H), 8.31 (d, J = 8.0 Hz, 1H), 8.15 (s, 1H), 7.53 (dd, J = 8.0 Hz, 4.0 Hz, 1H), 7.50 (d, J = 8.4 Hz, 1H), 7.41 (d, J = 8.4 Hz, 1H), 5.14 (t, J = 9.6 Hz, 1H), 2.51-2.40 (m, 1H), 2.18-2.08 |
| | | (m, 1H), 1.13-1.06 (m, 7H), 0.86 (d, J = 6.8 Hz, |
| | | 3H) |
| 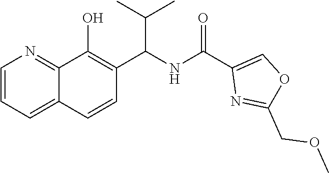 [SMILES: COCc1nc(C(=O)NC(c2ccc3cccnc3c2O)C(C)C)co1] | 5 | N-(1-(8-hydroxyquinolin-7-yl)-2-methylpropyl)-2- (methoxymethyl)oxazole-4-carboxamide LC-MS: m/z 356 (M + H)⁺. 1H NMR (400 MHz, CDCl3) δ (ppm): 8.77 (dd, J = 4.0 Hz, 1.6 Hz, 1H), 8.19 (d, J = 9.6 Hz, 1H), 8.15 (s, 1H), 8.13 (dd, J = 8.4 Hz, 1.6 Hz, 1H), 7.42 (d, J = 8.4 Hz, 1H), 7.41 (dd, J = 8.4 Hz, 4.4 Hz, 1H), 7.30 (d, J = 8.4 Hz, 1H), 5.13 (5, J = 9.6 Hz, 1H), 4.54 (s, 2H), 3.47 (s, 3H), 2.53-2.40 (m, 1H), 1.11 (d, J = 6.4 Hz, 3H), 0.88 (d, J = 6.4 Hz, 3H) |
| 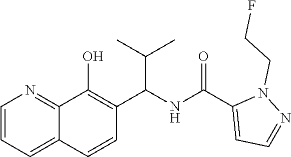 [SMILES: CC(C)C(NC(=O)c1ccnn1CCF)c1ccc2cccnc2c1O] | 5 | 1-(2-fluoroethyl)-N-(1-(8-hydroxyquinolin-7-yl)-2- methylpropyl)-1H-pyrazole-5-carboxamide LC-MS: m/z 357 (M + H)⁺. 1H NMR (400 MHz, DMSO-d6) δ (ppm): 9.76 (br s, 1H), 8.85 (dd, J = 4.0 Hz, 1.6 Hz, 1H), 8.70 (d, J = 9.2 Hz, 1H), 8.29 (dd, J = 8.4 Hz, 1.2 Hz, 1H), 7.67 (d, J = 8.4 Hz, 1H), 7.56-7.51 (m, 2H), 7.41 (d, J = 8.4 Hz, 1H), 6.98 (d, J = 2.0 Hz, 1H), 5.39 (5, J = 9.2 Hz, 1H), 4.84-4.52 (m, 4H), 2.24-2.11 (m, 1H), 1.04 (d, J = 6.8 Hz, 3H), 0.78 (d, J = 6.8 |
| | | Hz, 3H) |
| 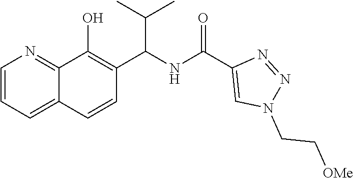 [SMILES: COCCn1cc(C(=O)NC(c2ccc3cccnc3c2O)C(C)C)nn1] | 5 | N-(1-(8-hydroxyquinolin-7-yl)-2-methylpropyl)-1- (2-methoxyethyl)-1H-1,2,3-triazole-4-carboxamide LC-MS: m/z 370 (M + H)⁺. 1H NMR (400 MHz, DMSO-d6) δ (ppm): 10.02 (br s, 1H), 8.87 (dd, J = 4.4 Hz, 1.6 Hz, 1H), 8.75 (d, J = 9.6 Hz, 1H), 8.48 (s, 1H), 8.34 (dd, J = 8.4 Hz, 1.6 Hz, 1H), 7.69 (d, J = 8.4 Hz, 1H), 7.56 (dd, J = 8.4 Hz, 4.4 Hz, 1H), 7.40 (d, J = 8.8 Hz, 1H), 5.29 (5, J = 9.6 Hz, 1H), 4.57 (t, J = 5.2 Hz, 2H), 3.73 (t, J = 5.2 Hz, 2H), 3.22 (s, 3H), 2.37-2.25 (m, 1H), 1.04 (d, J = 6.8 Hz, 3H), 0.77 (d, J = 6.8 Hz, |
| | | 3H) |
| 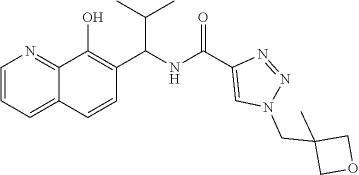 [SMILES: CC(C)C(NC(=O)c1cn(CC2(C)COC2)nn1)c1ccc2cccnc2c1O] | 5 | N-(1-(8-hydroxyquinolin-7-yl)-2-methylpropyl)-3- ((3-methyloxetan-3-yl)methyl)-3H-pyrazole-5- carboxamide LC-MS: m/z 395 (M + H)⁺. 1H NMR (400 MHz, DMSO-d6) δ (ppm): d 9.98 (br s, 1H), 8:86 (dd, J = 4.4 Hz, 1.6 Hz, 1H), 8.75 (d, J = 9.2 Hz, 1H), 8.61 (s, 1H), 8.32 (dd, J = 8.4 Hz, 1.6 Hz, 1H), 7.68 (d, J = 8.4 Hz, 1H), 7.55 (dd, J = 8.0 Hz, 4.0 Hz, 1H), 7.40 (d, J = 8.4 Hz, 1H), 5.29 (5, J = 9.6 Hz, 1H), 4.65 (s, 2H), 4.55 (t, J = 6.4 Hz, 2H), 4.23 (t, J = 6.4 Hz, 2H), 2.37-2.26 (m, |
| | | 1H), 1.14 (s, 3H), 1.03 (d, J = 6.8 Hz, 3H), 0.77 |
| | | (d, J = 6.8 Hz, 3H) |
| 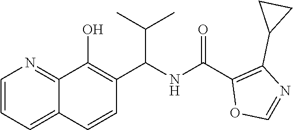 [SMILES: CC(C)C(NC(=O)c1ocnc1C1CC1)c1ccc2cccnc2c1O] | 5 | 4-cyclopropyl-N-(1-(8-hydroxyquinolin-7-yl)-2- methylpropyl)oxazole-5-carboxamide LC-MS: m/z 352 (M + H)⁺. 1H NMR (400 MHz, CD3OD) δ (ppm): 8:81-8.78 (m, 1H), 8.21 (d, J = 8.4 Hz, 1H), 8.05 (s, 1H), 7.50-7.44 (m, 2H), 7.34 (d, J = 8.0 Hz, 1H), 5.19 (d, J = 9.2 Hz, 1H), 2.65-2.56 (m, 1H), 2.46-2.38 (m, 1H), 1.12 (d, J = 6.8 Hz, 2H), 0.99-0.65 (m, |
| | | 4H), 0.86 (d, J = 6.8 Hz, 3H) |
| 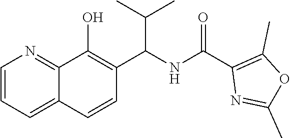 [SMILES: Cc1nc(C(=O)NC(c2ccc3cccnc3c2O)C(C)C)c(C)o1] | 5 | N-(1-(8-hydroxyquinolin-7-yl)-2-methylpropyl)- 2,5-dimethyloxazole-4-carboxamide LC-MS: m/z 340 (M + H)⁺. 1H NMR (400 MHz, CD3OD) δ (ppm): d 8.80 (dd, J = 4.4 Hz, 1.6 Hz, 1H), 8.23 (dd, J = 8.4 Hz, 1.6 Hz, 1H), 7.48 (dd, J = 8.0 Hz, 4.0 Hz, 1H), 7.45 (d, J = 8.0 Hz, 1H), 7.37 (d, J = 8.4 Hz, 1H), 5.12 (d, J = 9.2 Hz, 1H), 2.52 (s, 3H), 2.41 (s, 3H), 2.48-2.37 (m, 1H), 1.08 (d, J = 6.8 Hz, 3H), 0.87 (d, J = 6.8 |
| | | Hz, 3H) |

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating creatine transporter deficiency, comprising administering to a mammal in need thereof a therapeutically effective amount of a compound that increases transport of a substrate by a creatine transporter.

2. The method of claim 1, wherein the substrate is creatine or a salt thereof.

3. The method of claim 1, wherein the substrate is guanidinoacetic acid or a salt thereof.

4. The method of claim 1, wherein the substrate is 3-guanidinopropionic acid or a salt thereof.

5. The method of claim 1, wherein the substrate is 4-guanidinobutyric acid or a salt thereof.

6. The method of claim 1, wherein the substrate is guanidinoethane sulfonic acid or a salt thereof.

7. The method of any one of claims 1-6, wherein the creatine transporter is a mutant creatine transporter.

8. The method of claim 8, wherein the mutant creatine transporter is mutant SLC6A8.

9. The method of any one of claims 1-8, wherein the compound increases transport of the substrate into an endothelial cell.

10. The method of claim 9, wherein the endothelial cell is a brain endothelial cell.

11. The method of any one of claims 1-8, wherein the compound increases transport of the substrate into a gut epithelial cell, a brain cell, or a muscle cell.

12. The method of claim 11, wherein the compound increases transport of the substrate into a gut epithelial cell.

13. The method of claim 11, wherein the compound increases transport of the substrate into a brain cell.

14. The method of claim 11, wherein the compound increases transport of the substrate into a muscle cell.

15. The method of any one of claims 1-14, wherein the mammal is a male.

16. The method of any one of claims 1-14, wherein the mammal is a female.

17. The method of any one of claims 1-16, wherein the mammal is a primate, equine, bovine, ovine, feline, or canine.

18. The method of any one of claims 1-16, wherein the mammal is a human.

19. A method of treating creatine transporter deficiency, comprising administering to a mammal in need thereof a therapeutically effective amount of a compound that increases transport of a substrate across the blood-brain barrier by a creatine transporter.

20. The method of claim 19, wherein the substrate is creatine or a salt thereof.

21. The method of claim 19, wherein the substrate is guanidinoacetic acid or a salt thereof.

22. The method of claim 19, wherein the substrate is 3-guanidinopropionic acid or a salt thereof.

23. The method of claim 19, wherein the substrate is 4-guanidinobutyric acid or a salt thereof.

24. The method of claim 19, wherein the substrate is guanidinoethane sulfonic acid or a salt thereof.

25. The method of any one of claims 19-24, wherein the creatine transporter is a mutant creatine transporter.

26. The method of claim 25, wherein the mutant creatine transporter is mutant SLC6A8.

27. The method of any one of claims 19-26, wherein the mammal is a male.

28. The method of any one of claims 19-26, wherein the mammal is a female.

29. The method of any one of claims 19-28, wherein the mammal is a primate, equine, bovine, ovine, feline, or canine.

30. The method of any one of claims 19-28, wherein the mammal is a human.

31. A method of treating creatine transporter deficiency, comprising administering to a mammal in need thereof a therapeutically effective amount of a compound that increases transport of a substrate across the neuronal plasma membrane by a creatine transporter.

32. The method of claim 31, wherein the substrate is creatine or a salt thereof.

33. The method of claim 31, wherein the substrate is guanidinoacetic acid or a salt thereof.

34. The method of claim 31, wherein the substrate is 3-guanidinopropionic acid or a salt thereof.

35. The method of claim 31, wherein the substrate is 4-guanidinobutyric acid or a salt thereof.

36. The method of claim 31, wherein the substrate is guanidinoethane sulfonic acid or a salt thereof.

37. The method of any one of claims 31-36, wherein the creatine transporter is a mutant creatine transporter.

38. The method of claim 37, wherein the mutant creatine transporter is mutant SLC6A8.

39. The method of any one of claims 31-38, wherein the mammal is a male.

40. The method of any one of claims 31-38, wherein the mammal is a female.

41. The method of any one of claims 31-39, wherein the mammal is a primate, equine, bovine, ovine, feline, or canine.

42. The method of any one of claims 31-39, wherein the mammal is a human.

43. A method of decreasing accumulation or the concentration of guanidinoacetic acid or a salt thereof in a cell, comprising administering to a mammal in need thereof a therapeutically effective amount of a compound that increases transport of guanidinoacetic acid or a salt thereof by a creatine transporter.

44. The method of claim 43, wherein the compound decreases intracellular accumulation of guanidinoacetic acid or a salt thereof.

45. The method of claim 43, wherein the compound decreases the intracellular concentration of guanidinoacetic acid or a salt thereof.

46. The method of any one of claims 43-45, wherein the creatine transporter is a mutant creatine transporter.

47. The method of claim 46, wherein the mutant creatine transporter is mutant SLC6A8.

48. The method of any one of claims 43-436, wherein the cell is a brain cell.

49. The method of any one of claims 43-47, wherein the mammal is a male.

50. The method of any one of claims 43-47, wherein the mammal is a female.

51. The method of any one of claims 43-49, wherein the mammal is a primate, equine, bovine, ovine, feline, or canine.

52. The method of any one of claims 40-46, wherein the mammal is a human.

53. A method of increasing transport of guanidinoacetic acid or a salt thereof across the blood-brain barrier, comprising administering to a mammal in need thereof a therapeutically effective amount of a compound that increases transport of guanidinoacetic acid or a salt thereof by a creatine transporter.

54. The method of claim 53, wherein the creatine transporter is a mutant creatine transporter.

55. The method of claim 54, wherein the mutant creatine transporter is mutant SLC6A8.

56. The method of any one of claims 53-55, wherein the mammal is a male.

57. The method of any one of claims 53-55, wherein the mammal is a female.

58. The method of any one of claims 53-57, wherein the mammal is a primate, equine, bovine, ovine, feline, or canine.

59. The method of any one of claims 53-57, wherein the mammal is a human.
